(19) United States
(12) Patent Application Publication
(10) Pub. No.: US 2026/0146066 A1
(43) Pub. Date: May 28, 2026

(54) STOICHIOMETRIC GAS VESICLE EXPRESSION SYSTEM AND RELATED CONSTRUCTS, GENETIC CIRCUITS, VECTORS, CELLS, HOSTS, COMPOSITIONS, METHODS AND SYSTEMS

(71) Applicant: CALIFORNIA INSTITUTE OF TECHNOLOGY, Pasadena, CA (US)

(72) Inventors: Shirin Shivaei, Pasadena, CA (US); Mikhail G Shapiro, Los Angeles, CA (US)

(21) Appl. No.: 19/377,725

(22) Filed: Nov. 3, 2025

Related U.S. Application Data
(60) Provisional application No. 63/715,040, filed on Nov. 1, 2024.

(51) Int. Cl. C07K 14/195; A61K 49/22; C12N 15/86

(52) U.S. Cl. CPC C07K 14/195; A61K 49/223; C12N 15/86; C12N 2740/15043; C12N 2840/20

(57) ABSTRACT

Provided herein are stoichiometric Gas Vesicle Expression Systems (GVES), sets of polynucleotide constructs, and related vectors, cells, compositions, and methods configured for robust expression of Gas Vesicle Gene Clusters (GVGCs) in mammalian cells, particularly primary and immune cells. The GVES comprises distinct gene modules for the primary structural protein (gvpA/B) and assembly factors (AF1, AF2), operably configured to achieve a stoichiometric expression ratio, quantified by Dosage Index (DI), wherein the gvpA/B module is expressed at least 2-fold higher than the AF1 and AF2 modules.

### CROSS REFERENCE TO RELATED APPLICATIONS

The present application claims priority to U.S. Provisional Application No. 63/715,040, entitled “Ultrasound Imaging Of Gene Expression And Activation In Immune Cells” filed on Nov. 1, 2024, with docket number CIT 9230-P, incorporated herein by reference in its entirety. The present application is also related to U.S. patent application Ser. No. 16/736,683 filed on Jan. 7, 2020, (now U.S. Pat. No. 11,761,008), to U.S. application Ser. No. 16/736,581 filed on Jan. 7, 2020 (now U.S. Pat. No. 11,786,218), and PCT Application Number PCT/US2020/012557 filed on Jan. 7, 2020, the contents of each of which are also incorporated by reference in their entirety.

### STATEMENT OF GOVERNMENT GRANT

This invention was made with government support under Grant No. EB018975 awarded by the National Institutes of Health. The government has certain rights in the invention.

### INCORPORATION BY REFERENCE STATEMENT FOR SEQUENCE LISTING

Further, the computer readable form of the sequence listing of the ST26 XML file P3286-US-2025-11-03-Sequence Listing created on Nov. 3, 2025, and having size 614,924 bytes measured on Windows Server 2019 Datacenter is incorporated herein by reference in its entirety.

### FIELD

The present disclosure relates to gas-filled structures, and in particular to stoichiometric gas vesicle gene expression system, engineered gas vesicle polynucleotide constructs and related genetic circuits, vectors, genetically engineered mammalian cells, hosts, compositions, methods and systems, which can be used to produce robust expression of gas filled structures and/or to image biological events in a target site.

### BACKGROUND

Reporting biological events, such as gene expression, proteolysis, biochemical reactions as well as cell location and function, is currently primarily based on fluorescent reporter genes.

Even if alternative reporters, such as acoustic genetic reporters, have been identified, challenges remain for expressing these genes at the appropriate stoichiometry to achieve robust ultrasound contrast in particular in hard to transfect cells and/or when directed to efficiently deliver large, multi-gene clusters.

### SUMMARY

Provided herein are stoichiometric gas vesicle gene expression system, engineered gas vesicle polynucleotide constructs and related genetic circuits, vectors, genetically engineered mammalian cells, hosts, compositions, methods and systems which are configured to provide enhanced gas vesicles' yield and/or enhanced transfection efficiency for delivering the system to a target cell.

Accordingly stoichiometric gas vesicle gene expression system, engineered gas vesicle polynucleotide constructs and related genetic circuits, vectors, genetically engineered mammalian cells, hosts, compositions, methods and systems herein described can be used together with ultrasound and/or contrast-enhanced imaging techniques to achieve a robust detectable ultrasound contrast and/or to efficiently deliver the large, multi-gene cluster in mammalian cells including mammalian immune cells and primary cells.

According to a first aspect, a genetically engineered Gas Vesicle Expression System (GVES) is described, configured for expressing in a mammalian cell, a gene cluster of gvp genes (GVGC) encoding GV proteins capable of forming a GV type. The Gas Vesicle expression system comprises:
a gvpA/B differentially expressed gene module comprising a gvpA/B gene under control of a mammalian promoter and additional mammalian regulatory regions in a configuration allowing expression of a gvpA/B protein in the mammalian cell with a gvpA/B Dosage Index (DI), the
an AF1 gvp gene differentially expressed gene module for an AF1 set of gvp genes of the GV gene cluster encoding for a first set of GV assembly factors AF1 selected from gvpN, J, K, and/or F, under control of a mammalian promoter and additional regulatory regions in a configuration allowing expression of the GV assembly factors AF1 in the mammalian cell with a AF1 Dosage Index (DI); and
an AF2 gvp gene differentially expressed gene module comprising an AF2\set of gvp genes of the GV gene cluster encoding for a second set of GV assembly factors AF2 selected from gvpG, and optionally gvpW, and gvpV, under control of a mammalian promoter and additional regulatory regions in a configuration allowing expression of the GV assembly factors AF2 in the mammalian cell with a AF2 Dosage Index (DI).

In the Gas Vesicle expression system, each of the gvpA/B gene expression cassette, the AF1 gvp gene expression cassette and AF2 gene expression cassette, when comprising two or more gvp genes, further comprises a separation element between the two or more gvp genes configured to provide a separate expression of the corresponding GV protein.

In the Gas Vesicle expression system, the GVPB cassette and the one or more additional GVP cassettes are operably linked by regulatory sequences allowing co-expression of the GV proteins with a DI ratio of gvpA/B-DI:AF1-DI:AF2-DI wherein the gvpA/B-DI is at least 2-fold higher than the AF1-DI and the AF2-DI, thereby enabling formation of the GV type in the mammalian cell with enhanced yield.

In some preferred embodiments, the gvpA gene module, AF1 gene module, and the AF2 gene module are configured to obtain an effective DI ratio of AF1-DI to AF2-D2 h a gvpA/B-DI:AF1-DI:AF2-D2 an extended ratio ranging from 2:1:1 to 6:1:1 and formation of the GV type in the mammalian cell.

In addition or in the alternatives embodiments, the gvpA gene module, AF1 gene module, and the AF2 gene module are configured to obtain an effective DI ratio of AF1-DI to AF2-D2 which is less than 1:1 (This configuration, wherein the expression of the AF1 assembly factors is lower relative to the AF2 assembly factors, contributes to the overall enhanced yield of the GV type formation in the mammalian cell.

According to a second aspect, a set of GV polynucleotide constructs is described, configured for delivery into a mammalian cell of a gene cluster of gvp genes (GVGC) encoding GV proteins capable of forming a GV type. The set of polynucleotide constructs comprises:
a gvpA/B polynucleotide construct, comprising a gvpA/B differentially expressed gene module comprising a gvpA/B gene under control of a mammalian promoter and additional mammalian regulatory regions;
an AF1 GV polynucleotide construct, comprising an AF1 gvp differentially expressed gene module comprising an AF1 set of gvp genes of the GV gene cluster encoding for a first set of GV assembly factors AF1 selected from gvpN, J, K, and/or F, under control of a mammalian promoter and additional regulatory regions; and
an AF2 GV polynucleotide construct comprising an AF2 gvp differentially expressed gene module comprising an AF2 set of gvp genes of the GV gene cluster encoding for a second set of GV assembly factors AF2 selected from gvpG, and optionally gvpW, and gvpV, under control of a mammalian promoter and additional regulatory regions.

In the set of GV polynucleotide constructs, each of the AF1 gvp gene module and the AF2 gene module, when comprising two or more gvp genes, further comprises a separation element between the two or more gvp genes configured to provide separate expression of the corresponding GV proteins.

In the set of polynucleotide constructs, the gvpA/B gene module, the AF1 gene module, and the AF2 gene module are comprised on three separate GV polynucleotide constructs, wherein this specific construct configuration results in a Transduction-Expression Index (TEI) in mammalian cells that is at least 5-fold higher the TEI achieved by an expression system comprising said cassettes on a single polynucleotide construct under Standard Conditions, thus enabling efficient co-delivery and subsequent co-expression of the GVGC proteins.

In preferred embodiments, the set of vectors is provided as a composition wherein the relative ratio of transducing units for the first vector, second vector, and third vector is in an extended range from 2:1:1 to 4:1:1, enabling co-expression of the GV proteins and formation of the GV type in the mammalian cell.

In further preferred embodiments, the gvpA cassette, AF1 cassette, and the AF2 cassette are configured to obtain an effective DI ratio of AF1-DI to AF2-D2 which is less than 1:1 (e.g., 0.8:1, 0.5:1, or 0.2:1), This configuration, wherein the expression of the AF1 assembly factors is lower relative to the AF2 assembly factors, contributes to the overall enhanced yield of the GV type formation in the mammalian cell.

According to a third aspect, a genetically engineered mammalian Gas Vesicle Reporting molecular component (GVRMC) is described. The gas vesicle reporting molecular component comprises
at least one of the Gas Vesicle expression system (GVES) and the set of Gas Vesicle polynucleotide construct (GVPCs) herein described in which the mammalian regulatory regions comprise a gas vesicle reporting (GVR) target region configured to be activated and/or inhibited by a molecular component of a genetic circuit;
wherein the gvp genes and mammalian regulatory regions are in a configuration allowing expression of GV proteins encoded by the gvp genes through activation and/or inhibition of the gas vesicle reporting (GVR) target region, when the genetic circuit operates according to the circuit design in the mammalian cell.

According to a fourth aspect, a genetically engineered gas vesicle reporting (GVR) genetic circuit (GVRGC) configured for expression in a mammalian cell is described. In the GVR genetic circuit molecular components are connected one to another in a mammalian cell in accordance with a circuit design by activating, inhibiting, binding or converting reactions to form a fully connected network of interacting components.

The GVR genetic circuit comprises a mammalian Gas Vesicle Reporting Molecular Component (GVRMC) herein described in a configuration in which GV proteins encoded by the gvp genes of the GVRMC are expressed and a gas vesicle (GV) type is provided when the genetic circuit operates according to the circuit design.

According to a fifth aspect, a method to express a Gas Vesicles in a mammalian cell is described. The method comprises introducing into the mammalian cell a genetically engineered Gas Vesicle expression system (GVES) herein described for a time and under condition to allow expression of GV proteins encoded by the gvp genes of the GVES and production of the Gas vesicle type in the mammalian cell.

According to a sixth aspect, a genetically engineered mammalian cell is described comprising the Gas Vesicle expression system (GVES) and/or Gas Vesicle Polynucleotide Construct (GVPC) herein described, configured for expression in the genetically engineered mammalian cell.

According to a seventh aspect, a method to provide a gas vesicle in a mammalian host is described. The method comprises introducing into a cell of the mammalian host the genetically engineered Gas Vesicle expression system (GVES), the introducing performed for a time and under condition to allow expression of the GV proteins encoded by the gvp genes of the GVES and the production of the Gas Vesicle type in the mammalian cell.

According to an eighth aspect, a genetically engineered non-human mammalian host is described comprising the Gas Vesicle expression system (GVES) and/or Gas Vesicle Polynucleotide Construct (GVPC) herein described, configured for expression in a mammalian cell of the GV proteins encoded by the gvp genes of the GVES and the production of the Gas Vesicle type in the genetically engineered non-human mammalian host.

According to a ninth aspect, a method and system to provide a genetically engineered a mammalian cell comprising a GVR genetic circuit is described, the method comprising:
genetically engineering the mammalian cell to introduce into the mammalian cell one or more genetically engineered Gas Vesicle Reporting Molecular Components (GVRMC) herein described
   wherein at least one of the gvpB gene expression cassette and one or more additional gvp gene expression cassettes comprise a gas vesicle reporting (GVR) target region configured to be activated and/or inhibited by a molecular component of the GVR genetic circuit, to provide a Gas Vesicle Reporting Genetic Circuit (GVRGC) herein described.

According to a tenth aspect, a method is described to image a biochemical event in a mammalian cell comprised in an imaging target site, the method comprising:
introducing into the mammalian cell a Gas Vesicle Reporting Molecular Components (GVRMC) herein described to provide a GVR genetic circuit in which expression of GV proteins encoded by the gvp genes of the GVRMC and production of the the GV type or an intracellular spatial translocation of the GV type occurs when the GVR genetic circuit operates according to the circuit design in response to the biochemical event,
the introducing performed for a time and under conditions allowing expression of the GV proteins and production of the GV type or an intracellular spatial translocation of the GV type in response to the biochemical event; and
imaging the target site comprising the mammalian host by applying a magnetic field and/or ultrasound to obtain an MRI and/or an ultrasound image of the target site.

The system comprises the genetically engineered Gas Vesicle expression system (GVES), Gas Vesicle Polynucleotide Construct (GVPC), Gas Vesicle Reporting Molecular Components (GVRMC) and/or GVR genetic circuits (GVRGC), related components and/or mammalian host cells in a combination for simultaneous combined or sequential use in the imaging methods herein described.

According to an eleventh aspect, a method is described to label a target mammalian host, the method comprising:
introducing into the mammalian cell a Gas Vesicle Reporting Molecular Components (GVRMC) herein described to provide a GVR genetic circuit in which expression of GV proteins encoded by the gvp genes of the GVRMC and production of the GV type or an intracellular spatial translocation of the GV type occurs when the GVR genetic circuit operates according to the circuit design in response to a trigger molecular component;
   In the method, the introducing is performed under conditions resulting in presence of the trigger molecular component in the target mammalian host.
   In some embodiments, the method can further comprise imaging the target site comprising the target mammalian host, by applying a magnetic field and/or ultrasound to obtain an MRI and/or an ultrasound image of the target site.
   The system comprises the genetically engineered GVES, GVPC, related polynucleotide constructs, GVR genetic circuits, related components and/or mammalian host cells in a combination for simultaneous combined or sequential use in the imaging methods herein described.

According to a twelfth aspect, a composition is described. The composition comprises a genetically engineered Gas Vesicle expression system (GVES), set Gas Vesicle Polynucleotide Construct (GVPC), Gas Vesicle Reporting Molecular Components (GVRMC) and/or GVR genetic circuits (GVRGC) of the disclosure, vectors, and/or genetically engineered mammalian cells described herein together with a suitable vehicle.

The stoichiometric Gas Vesicle expression system (GVES), Gas Vesicle Polynucleotide Construct (GVPC), Gas Vesicle Reporting Molecular Components (GVRMC) GVR genetic circuits (GVRGC), related vectors, genetically engineered mammalian cells, compositions, methods and systems provide enhanced Gas Vesicle (GV) yield, resulting in a BURST Signal-to-Background Ratio (SBR) demonstrating a substantial improvement, over known system where the modules have different stoichiometric configurations s achieving in some cases at least a 20-fold increase and up to 30-fold higher values (e.g., as observed when comparing a multi-construct system to a single-construct system in HEK293T cells in vitro), and/or improved delivery efficiency, achieving a Transduction-Expression Index (TEI) at least 20-fold higher and in some cases up to 30-fold higher than known system comprising the GVGC modules configured according to a stoichiometry different from the stoichiometric arrangements of the disclosure. These systems are characterized by a Dosage Index (DI) ratio (DI_gvpA/B:DI_AF1:DI_AF2) of at least 2:1:1, offering significantly higher ultrasound sensitivity and broader applicability across cell types, including primary cells.

In particular, in several embodiments described herein, the stoichiometric Gas Vesicle expression system (GVES), Gas Vesicle Polynucleotide Construct (GVPC), Gas Vesicle Reporting Molecular Components (GVRMC) GVR genetic circuits (GVRGC), related vectors, genetically engineered mammalian cells, compositions, methods and systems, can be used to report the location of mammalian cells with greater sensitivity, quantifiable for example by achieving a BURST SBR greater than 75, and preferably greater than 100 (e.g., as observed in HEK293T cells in vitro), which results from the stoichiometric optimization (characterized by a DI ratio of at least 2:1:1), and/or sense and report more reliably, particularly in clinically relevant cell types enabled by the multi-construct delivery architecture (which achieves high transduction (e.g., ˜55-60% double-positive in HEK293T cells) while showing no significant impact on cell viability (Toxicity/Viability Index ˜100%, not significantly different from controls) or important primary T cell functions like cytotoxicity).

The stoichiometric Gas Vesicle expression system (GVES), Gas Vesicle Polynucleotide Construct (GVPC), Gas Vesicle Reporting Molecular Components (GVRMC) GVR genetic circuits (GVRGC), related vectors, genetically engineered mammalian cells, compositions, methods and systems, can be used in several embodiments to allow multiplexed imaging wherein the stoichiometric control, which is shown to be a result effective variable for maximizing signal (e.g., achieving a BURST SBR greater than 75, and in some cases greater than 100, by establishing a gvpA:AF1:AF2 DI ratio of at least 2:1:1 in HEK293T cells in vitro), provides improved reliability and a wide functional dynamic range (e.g., a 4.1-fold difference in vivo between induced and uninduced states and a 10.9-fold difference in vitro upon chemical activation of T cells).

The stoichiometric Gas Vesicle expression system (GVES), Gas Vesicle Polynucleotide Construct (GVPC), Gas Vesicle Reporting Molecular Components (GVRMC) GVR genetic circuits (GVRGC), related vectors, genetically engineered mammalian cells, compositions, methods and systems, can be used in several embodiments to detect events such as multiple gene expression wherein the enhanced GV yield, which correlates with a high Assembly Efficiency (AE) of functional particles to produce a BURST SBR greater than 75, and preferably greater than 100, in vitro, improves the sensitivity of detection. This high-yield system is achieved through stoichiometric optimization (characterized by a DI ratio of gpvpA/B-DI:AF1-DI:AF2-D2 of at least 2:1:1, and in certain embodiments, wherein the effective DI ratio of AF1-DI to AF2-D2 is less than 1:1).

The stoichiometric Gas Vesicle expression system (GVES), Gas Vesicle Polynucleotide Construct (GVPC), Gas Vesicle Reporting Molecular Components (GVRMC) GVR genetic circuits (GVRGC), related vectors, genetically engineered mammalian cells, compositions, methods and systems, can be used in several embodiments to report biochemical events with sensitivity enhanced by the system's optimized architecture, which provides high functional output (e.g., a BURST SBR greater than 75, and preferably greater than 100, in vitro) and efficient delivery to primary cells (e.g., primary human T cells), providing a wide functional dynamic range. demonstrated for example by a 4.1-fold difference in BURST SBR in vivo between doxycycline-induced and uninduced primary T cells, and a 10.9-fold difference in BURST SBR in vitro upon chemical activation of engineered Jurkat T cells.

The stoichiometric Gas Vesicle expression system (GVES), Gas Vesicle Polynucleotide Construct (GVPC), Gas Vesicle Reporting Molecular Components (GVRMC) GVR genetic circuits (GVRGC), related vectors, genetically engineered mammalian cells, compositions, methods and systems, can be used in several embodiments to produce dynamic contrast. reflecting cell presence and/or activity. This quantitative capability is demonstrated, for example, by the strong positive linear correlation (r=0.89) observed between in vivo BURST signal and the number of reporter-positive T cells counted in tumors using a multi-construct delivery approach. The stoichiometric optimization of the system, characterized by DI ratios achieving macrooptimization (e.g., gvpA/B-DI at least 2-fold higher than AF1-DI and AF2-DI) and potentially microoptimization (e.g., AF1-DI<AF2-DI), is known or expected to contribute to the high signal yield that facilitates such quantitative measurements.

The stoichiometric Gas Vesicle expression system (GVES), Gas Vesicle Polynucleotide Construct (GVPC), Gas Vesicle Reporting Molecular Components (GVRMC) GVR genetic circuits (GVRGC), related vectors, genetically engineered mammalian cells, compositions, methods and systems can be used in several embodiments to provide ultrasound imaging, allowing for detection of gas vesicle-expressing cells at volumetric concentrations of approximately 0.9% in vitro (corresponding to 1e6 cells/mL), including in hard-to-transduce primary cells enabled by the multi-construct delivery system, and/or to image gene expression in mammals in vivo using ultrasound with higher sensitivity corresponding to a 4:1-fold to 4.3-fold higher BURST SBR compared to uninduced or wild-type T cell controls respectively.

The stoichiometric Gas Vesicle expression system (GVES), Gas Vesicle Polynucleotide Construct (GVPC), Gas Vesicle Reporting Molecular Components (GVRMC) GVR genetic circuits (GVRGC), related vectors, genetically engineered mammalian cells, compositions, methods and systems, can be used in several embodiments to track presence location and/or expansion of mammalian cells and are particularly suitable for tracking therapeutic cells, such as primary T-cells or CAR-T cells, due to the efficient delivery (achieving a sorted population where ˜56% of primary T cells are positive for both gvpA and assembly factor reporters) enabled by the multi-construct architecture and the high signal (e.g., an in vitro BURST SBR 3.8-fold higher than wild-type T-cell controls). These system features are characterized by a high Transduction-Expression Index (TEI), evidenced by. approximately a 4.1-fold to 4.3-fold in vivo SBR increase over uninduced or wild-type controls, respectively.

The stoichiometric Gas Vesicle expression system (GVES), Gas Vesicle Polynucleotide Construct (GVPC), Gas Vesicle Reporting Molecular Components (GVRMC) GVR genetic circuits (GVRGC), related vectors, genetically engineered mammalian cells, compositions, methods and systems, can in some embodiments be used to allow measures of fluid flows, wherein the stronger signal, quantifiable by an in vitro BURST SBR over 100, can improve tracking accuracy. This stronger signal results from the stoichiometric optimization of the system (characterized by an optimized DI ratio of gpvpA/B-DI:AF1-DI:AF2-D2 of at least 2:1:1, and in certain embodiments, wherein the effective DI ratio of AF1-DI to AF2-D2 is less than 1:1).

The stoichiometric Gas Vesicle expression system (GVES), Gas Vesicle Polynucleotide Construct (GVPC), Gas Vesicle Reporting Molecular Components (GVRMC) GVR genetic circuits (GVRGC), related vectors, genetically engineered mammalian cells, compositions, methods and systems, can be used in connection with various applications wherein reporting, labeling, and/or tracking is desired with high sensitivity (e.g., achieving an in vivo detection limit sufficient to track T cell homing to tumors and an in vitro detection limit of 1e6 cells/mL) and efficiency (e.g., successfully transducing hard-to-transduce primary T-cells with no significant impact on critical cell functions like cytotoxicity).

For example, stoichiometric Gas Vesicle expression system (GVES), Gas Vesicle Polynucleotide Construct (GVPC), Gas Vesicle Reporting Molecular Components (GVRMC) GVR genetic circuits (GVRGC), related vectors, genetically engineered mammalian cells, compositions, methods and systems, can be used for visualization of biological events, including the monitoring of cell-based therapies like engineered T-cells trafficking to tumors with improved detection limits enabled by the system's high-yield (providing a 4.3-fold in vivo SBR over controls and a strong correlation (r=0.89) with cell numbers) and high-efficiency (enabling non-invasive tracking of sorted primary T-cells).

The stoichiometric Gas Vesicle expression system (GVES), Gas Vesicle Polynucleotide Construct (GVPC), Gas Vesicle Reporting Molecular Components (GVRMC) GVR genetic circuits (GVRGC), related vectors, genetically engineered mammalian cells, compositions, methods and systems, can also be used in developmental biology, stem cells, and iPSC derived cells, offering improved suitability for these applications due to the enhanced GV yield. This enhanced yield is a result of the system's stoichiometric optimization (characterized by an optimized DI ratio of gpvpA/B-DI:AF1-DI:AF2-D2 of at least 2:1:1, and in certain embodiments, wherein the effective DI ratio of AF1-DI to AF2-D2 is less than 1:1), which provides a >4-fold SBR in vivo and a >30-fold SBR in vitro over single-vector systems. The system also provides efficient delivery capabilities (achieving functional expression in primary T-cells without impairing cytotoxicity), particularly for primary and immune cell-based agents).

Additional exemplary applications include uses of the stoichiometric GVES, and related polynucleotide constructs, GVR genetic circuits, vectors, genetically engineered mammalian cells, genetically engineered non-human mammals, compositions, methods and systems herein described in several fields including basic biology research, applied biology, bio-engineering, bio-energy, medical research, medical diagnostics, therapeutics, and in additional fields identifiable by a skilled person upon reading of the present disclosure.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and the examples, serve to explain the principles and implementations of the disclosure.

FIG. 1 shows an exemplary Clustal omega alignment of amino acid sequences of selected exemplary gvpA and gvpB proteins (SEQ ID NO: 7-10 and 457-472).

FIG. 2 shows exemplary phylogenetic relationships of the gvpA protein sequences from the indicated prokaryotic species. [1]

FIG. 3 shows exemplary phylogenetic relationships of the gvpF and gvpL protein sequences from the indicated prokaryotic species. [1]

FIG. 4 shows exemplary phylogenetic relationships of the gvpN protein sequences from the indicated prokaryotic species. [1]

FIG. 5 shows diagrams illustrating the organization of exemplary gas vesicle gene clusters. Gas vesicle gene clusters from the indicated organisms are shown, with genes shown as block-shaped arrows, and genes of predicted similar function indicated in the same shade of grey. The direction of the transcription of genes within a gene cluster is indicated by the direction of the block-shaped arrows, and genes grouped together having block arrows pointed in the same direction are typically organized in the same operon. The scale bar indicates 1 kb. [1]

FIG. 6 shows diagrams illustrating organization of exemplary gvp gene clusters, wherein each letter indicates a gvp gene, and an arrow beneath a group of letters indicates an operon, with the direction of the arrow indicating the direction of transcription. [2]

FIGS. 7A-7L: Design and optimization of multi-vector lentiviral delivery systems for robust GV expression and ultrasound imaging. FIG. 7A, Schematic of the viral production and screening process. HEK293T cells are transduced with lentiviral vectors encoding GV genes, followed by doxycycline-induced GV expression for 72 hours. Cells are detached and embedded in agarose phantoms for ultrasound imaging. FIG. 7B, Schematic of a single lentiviral vector encoding all 8 GV genes. FIG. 7C, Representative BURST ultrasound images of HEK-TetON cells transduced with the single-vector virus at various multiplicities of infection (MOIs). FIG. 7D, Quantification of BURST signal-to-background ratio (SBR) in cells transduced with the single-vector virus, normalized to non-transduced cells. FIG. 7E, Schematic of two lentiviral vectors encoding GV genes and the rtTA transactivator. FIG. 7F, Representative BURST images of HEK293T cells transduced with the two-vector system at a total MOI of 10, with varying ratios of gvpA virus to assembly factor (AF) virus. FIG. 7G, Quantification of BURST SBR in cells from transduced with (e). FIG. 7H, Schematic of a three-vector system encoding the GV genes. FIG. 7I, Representative BURST images of HEK293T cells transduced with the three-vector system at a total MOI of 10, with varying ratios of gvpA to the first assembly factor (AF1) virus and the second assembly factor (AF2) virus. [3] 7J, Quantification of BURST SBR in cells transduced with (h). FIG. 7K, Representative BURST images (top) and SBR (bottom) of HEK293T cells transduced with the three-vector system at a 4:1:1 ratio and sorted by fluorescence, shown as a function of cell concentration. FIG. 7L, Representative xAM images (top) and corresponding SBR (bottom) of the same cells as panel (k), shown as a function of cell concentration. For panels (d, g, j), statistical comparisons to non-transduced controls were performed using Fisher's least significant difference (LSD) test. For panels (k-l), comparisons were made against the “no cells” control using Kruskal-Wallis test. P-values from left to right: (d): 0.0017, 0.0165, 0.0333; (g): 0.0006, <0.0001, 0.0004; (j): 0.0107, <0.0001, <0.0001; (k): 0.0137, 0.001; (1): 0.0285, 0.0026. Significance levels: * p<0.05, ** p<0.01, *** p<0.001, **** p<0.0001; nonsignificant data points are not marked. For (d, g, j), error bars represent mean #s.e.m. of N=3 biological replicates. Each data point reflects the arithmetic mean of N=2 technical replicates, unless noted otherwise. For (k, 1), N=3 replicates from independent dilution series. Scale bars for all ultrasound images represent 1 mm. au: arbitrary units. Dox: doxycycline.

FIG. 8A-8E: Adaptability of lentiviral GV gene delivery and ultrasound imaging of immune cells. FIG. 8A, Schematic of doxycycline-inducible GV expression in Jurkat cells transduced with three lentiviral vectors encoding the GV gene cluster. FIG. 8B, Representative BURST ultrasound images of Jurkat cells transduced with different total MOIs and three ratios of gvpA to assembly factor (AF1 and AF2) viruses. FIG. 8C, Quantified BURST signal in Jurkat cells from panel (b), normalized to wild-type (WT) Jurkat cells. FIG. 8D, Representative BURST images (top) and corresponding signal quantification (bottom) of Jurkat cells transduced with the three-vector lentivirus system (MOI 60) at a 4:1:1 ratio. Cells were sorted by fluorescence expression and imaged at varying cell concentrations to measure imaging detection limit. FIG. 8E, Representative xAM images (top) and signal quantification (bottom) of the same sorted cell line from panel (d), shown as a function of cell concentration. Statistical comparisons for panels (d) and (e) were made using Kruskal-Wallis test, with each condition compared to the “no cells” control. N=3 replicates from independent dilution series. P-values for (d) from left to right: 0.0285, 0.001; (e): 0.0026. Significance levels: * p<0.05, ** p<0.01, *** p<0.001, **** p<0.0001. Error bars in (c) represent mean #s.e.m. of N=3 biological replicates. Each data point is the arithmetic mean of N=2 technical replicates. For (d, e), N=3 replicates from independent dilution series. All ultrasound image scale bars represent 1 mm.

FIGS. 9A-9F. Activation-dependent GV expression enables ultrasound imaging of T cell activity. FIG. 9A, Schematic of lentiviral vectors designed to report T cell activity state by expressing GVs downstream of the NFAT promoter. T cell activation is chemically induced by incubating cells with PMA and ionomycin, which trigger the NFAT promoter to express rtTA, and in the presence of doxycycline, activate transcription of GV genes. FIG. 9B, Representative BURST images (top) and signal quantification (bottom) of Jurkat cells transduced with the vectors in panel (a) with or without chemical activation, normalized to wild-type (WT) cells (not shown). FIG. 9C, Percentage of Jurkat cells in panel (b) expressing both fluorescent reporters (GFP and BFP), with and without activation. FIG. 9D, Imaging T cell activation in response to CD19-CD3 BTE-mediated engagement with CD19+ Raji cells, using cells engineered with the vectors shown in (a). FIG. 9E, Representative BURST images (top) and signal quantification (bottom) of Jurkat cells engineered to express GVs upon receptor-mediated activation in the presence of Dox. FIG. 9F, Percentage of cells from panel (e) expressing both fluorescent reporters (GFP and BFP), with and without receptor engagement. Statistical comparisons were performed using Fisher's LSD test, with each condition compared to resting state T cells (without PMA/ionomycin or BTE stimulation). P-value for (e): 0.0173. All other p-values <0.0001. Significance levels: *p<0.05, ** p<0.01, *** p<0.001, **** p<0.0001. Error bars represent mean±s.e.m. of N=3 biological replicates. Each data point represents the arithmetic mean of N=2 technical replicates. Scale bars for all ultrasound images represent 1 mm.

FIGS. 10A-10I: In vivo tracking and functional assessment of primary T cells engineered to express GVs. FIG. 10A, Workflow for engineering T cells isolated from human PBMCs to express doxycycline-inducible GVs and imaging BTE-mediated homing of cytotoxic T cells into target tumors. Top: T cells are transduced with the 3-vector lentiviral system, and GFP expressing cells are sorted to select for transduction of the assembly factor vectors. The cells are then expanded in vitro for downstream characterization and in vivo injections. Bottom: T cells are systemically administered to immunocompromised mice bearing subcutaneous CD19+ Raji cell tumors, accompanied by intraperitoneal (IP) injections of BTE every 48 hours and IP injections of doxycycline for 72 hours prior to ultrasound imaging. FIG. 10B, BURST SBR (left) and representative images (right) of GV-expressing and WT T cells. Paired one-tailed t test, p=0.0115. Error bars represent mean±s.e.m. of N=5 PBMC donors; each data point is the arithmetic mean of N=3 technical replicates. FIG. 10C, Percentage of live Raji cells in a cytotoxicity assay, where T cells are co-cultured with CD19+ Raji cells at effector-to-target (E:T) ratios of 1:1 and 4:1. Welch's two-tailed t-test, N=6 biological replicates (2 PBMC donors and 3 replicates per donor). FIG. 10D, GFP and BFP fluorescence measurements from (c), normalized to the maximum mean fluorescence intensity (MFI) of each reporter. FIG. 10E, BURST images overlaid on anatomical B-mode grayscale images of representative Raji tumors in mice injected with GV-expressing T cells, either Dox-induced or uninduced, and mice injected with WT T cells. Red lines indicate tumor boundary used for signal quantification. FIG. 10F, Quantification of BURST signal inside tumors. Welch's two-tailed t test, Dox+ vs. Dox−, p=0.0067; Dox+ vs. WT, p=0.0063. N=10 mice for Dox+, N=6 for Dox−, N=6 for WT. FIG. 10G, Histology of an example tumor infiltrated by GV-expressing T cells and its corresponding BURST image. White: Raji-Antares cells; Magenta: CD8+ cytotoxic T cells; Green: GFP+ T cells; Blue: BFP+ T cells. FIG. 10H, Schematic of T cell distribution in the Dox-induced tumor from (g). FIG. 10I, Correlation of the BURST signal within tumors with the absolute numbers of GFP+ and BFP+ T cells in corresponding histological sections. Pearson correlation: r=0.89, p=0.0072, N=7 mice. One mouse (gray data point) was excluded due to a significant number of T cells located outside the imaging plane whose GV expression was not captured by BURST (see FIG. 18). All scale bars represent 1 mm.

FIG. 11A-11C: xAM imaging of HEK293T cells expressing GVs using different lentiviral vector designs. FIG. 11A, xAM imaging of HEK-TetON cells transduced with the single lentiviral vector encoding all necessary GV genes, shown at increasing MOIs. FIG. 11B, xAM imaging of HEK293T cells transduced with the two-vector lentiviral system at increasing ratios of gvpA to assembly factors (AF). FIG. 11C, xAM imaging of HEK293T cells transduced with the three-vector lentiviral system at increasing ratios of gvpA to AF1 and AF2. For all panels, N=3 biological replicates. Each data point is the arithmetic mean of N=2 technical imaging replicates. All ultrasound image scale bars represent 1 mm.

FIG. 12: Flow cytometry characterization of HEK293T cells transduced with various lentiviral designs encoding GV genes. a-b, Percentage of HEK-TetON cells transduced with the single-vector virus that express GFP, the marker for transduction and their mean fluorescence intensity (MFI). c-d, Percentage of HEK293T cells transduced with the two-vector lentivirus system, expressing both GFP and BFP, markers for double transduced cells, and their MFI. e-f, Percentage of HEK293T cells transduced with the three-vector lentivirus system expressing GFP and BFP, markers for triple transduced cells, and their MFI. g, Percentage of live cells transduced with the three-vector lentivirus system at a 4:1:1 ratio (gvpA:AF1:AF2) and sorted for triple-transduction, compared to cells transduced with a control virus constitutively expressing BFP at the same total MOI of 10, and WT cells. There is no significant difference in viability between GV-expressing cells (Dox+), uninduced cells (Dox-), the BFP control line, or wild-type (WT) cells (statistical comparisons were made using ordinary one-way ANOVA). N=3 biological replicates.

FIG. 13: xAM imaging of Jurkat cells transduced with the 3-vector lentivirus system at varying MOIs. a-b, xAM imaging of Jurkat cells transduced with three lentiviral vectors encoding all necessary GV genes, shown at different vector ratios and total MOIs of 30 and 60. Error bars represent mean #s.e.m. N=3 biological replicates. Each data point represents the arithmetic mean of N=2 technical imaging replicates. All ultrasound image scale bars represent 1 mm.

FIG. 14: Flow cytometry analysis of Jurkat cells expressing GVs. a, Percentage of Jurkat cells transduced with all three GV lentiviruses that express both GFP and BFP. b-c, Mean fluorescence intensity (MFI) of GFP and BFP expression in the cells from panel (a). d-e, Cell viability of Jurkat cells transduced with GV-expressing lentiviruses at MOIs of 30 (d) and 60 (e), with and without doxycycline induction, compared to a control virus constitutively expressing BFP at the same MOI. The viability of cells transduced with the BFP virus (packaging size 5.1 kb) was not significantly affected by MOI. Smaller viral transgenes achieve higher functional titers per capsid, requiring less viral volume to achieve the same functional titer compared to larger viruses (e.g., AF1 from the 3-vector system at 6.7 kb), resulting in lower toxicity for the BFP virus overall. N=3 biological replicates.

FIG. 15: Characterization of sorted T cell subpopulations using fluorescent transduction markers. a, Flow cytometry analysis of doxycycline-induced and sorted T cells shows that the majority of T cells express both lentiviral transduction markers, GFP and BFP, four days after sorting. As the cells were sorted mainly based on GFP expression (assembly factor viral vectors), a smaller subpopulation expresses only GFP without BFP, the marker for the gvpA-encoding viral vector. N=5 PBMC donors.

FIG. 16: Ultrasound imaging of Raji tumors infiltrated with T cells. Representative BURST images (colormap) overlaid on anatomical B-mode images (grayscale) of subcutaneous Raji tumors, with each image representing a different mouse. a, Tumors from mice infused with virally transduced T cells, induced with doxycycline for 3 days to express GVs. b, Tumors from mice infused with transduced T cells but left uninduced. c, Tumors from mice infused with wild-type (WT) T cells.

FIG. 17: Histological validation of T cell infiltration in tumors. a, Ultrasound images (BURST overlaid on B-mode) of doxycycline-induced, GV-expressing T cells in Raji-Antares tumors, alongside corresponding histology showing cytotoxic CD8+ T cells, GFP+ T cells, and BFP+ T cells. Mice 7 and 8 were not processed for anti-CD8 staining due to a negligible presence of GFP/BFP-expressing T cells. Note that the histology and ultrasound images are not an exact spatial match, but are within 1 mm of each other. White: Antares; Magenta: anti-CD8; Green: GFP; Blue: BFP. All scale bars represent 1 mm.

FIG. 18: Out-of-focus partial collapse in BURST imaging with a linear array transducer. BURST imaging with a linear array transducer generates a focused ultrasound beam, with the highest acoustic pressure at the center of the focus (zone 1). One limitation of this method is the potential for collapsing GVs outside the focus of imaging (zone 2) at lower pressures, without producing a detectable BURST signal. While varying the focus to different depths allows us to capture some of the collapse-based nonlinear scattering, this method of imaging may not be perfectly capturing GV expression throughout the entire tumor. A potential solution would be to use a matrix array or row-column array transducer, which can generate a consistent axial pressure field along the ultrasound beam.

### DETAILED DESCRIPTION

Provided herein are genetically engineered gas vesicle expression systems (GVES) and related polynucleotide constructs configured for expression of a gas vesicle (GV) in a mammalian cell, and related gas vesicle gene clusters, gas vesicles, genetic circuits, vectors, genetically engineered mammalian cells, compositions, methods and systems.

The wordings “gas vesicles”, GV″, “gas vesicles protein structure”, or “GVPS”, refer to a gas-filled protein structure natively intracellularly expressed by certain bacteria or archaea as a mechanism to regulate cellular buoyancy in aqueous environments [4]. In particular, gas vesicles are protein structures natively expressed almost exclusively in microorganisms from aquatic habitats, to provide buoyancy by lowering the density of the cells [4]. GVs have been found in over 150 species of prokaryotes, comprising cyanobacteria and bacteria other than cyanobacteria [5, 6], from at least 5 of the 11 phyla of bacteria and 2 of the phyla of archaea described by Woese (1987) [7]. Exemplary microorganisms expressing or carrying gas vesicle protein structures and/or related genes include cyanobacteria such as Microcystis aeruginosa, Aphanizomenon flos aquae Oscillatoria agardhii, Anabaena, Microchaete diplosiphon and Nostoc; phototropic bacteria such as Amoebobacter, Thiodiclyon, Pelodiclyon, and Ancalochloris; non phototropic bacteria such as Microcyclus aquaticus; Gram-positive bacteria such as Bacillus megaterium Gram-negative bacteria such as Serratia; and archaea such as Haloferax mediterranei, Methanosarcina barkeri, and Halobacteria salinarium, as well as additional microorganisms identifiable by a skilled person.

In particular, a GV in the sense of the disclosure is an intracellularly expressed structure forming a hollow structure wherein a gas is enclosed by a protein shell, which is a shell substantially made of protein (at least 95% protein). In gas vesicles in the sense of the disclosure, the protein shell is formed by a plurality of proteins herein also indicated as GV proteins or gvps, which form in the cytoplasm a gas permeable and liquid impermeable protein shell configuration encircling gas. Accordingly, a protein shell of a GV is permeable to gas but not to surrounding liquid such as water. In particular, GV protein shells exclude water but permit gas to freely diffuse in and out from the surrounding media [8] making them physically stable despite their usual nanometer size, unlike microbubbles, which trap pre-loaded gas in an unstable configuration.

GV structures are typically nanostructures with widths and lengths of nanometer dimensions (in particular with widths of 45-250 nm and lengths of 100-800 nm) but can have lengths up to 2 μm in prokaryotes but can have larger dimensions such as up to 8-10 μm as will be understood by a skilled person upon reading of the present disclosure. In certain embodiments, the gas vesicles protein structure have average dimensions of 1000 nm or less, such as 900 nm or less, including 800 nm or less, or 700 nm or less, or 600 nm or less, or 500 nm or less, or 400 nm or less, or 300 nm or less, or 250 nm or less, or 200 nm or less, or 150 nm or less, or 100 nm or less, or 75 nm or less, or 50 nm or less, or 25 nm or less, or 10 nm or less. For example, the average diameter of the gas vesicles may range from 10 nm to 1000 nm, such as 25 nm to 500 nm, including 50 nm to 250 nm, or 100 nm to 250 nm. By “average” is meant the arithmetic mean.

GVs in the sense of the disclosure have different shapes depending on their genetic origins [8]. For example, GVs in the sense of the disclosure can be substantially spherical, ellipsoid, cylindrical, or have other shapes such as football shape or cylindrical with cone shaped end portions depending on the type of bacteria providing the gas vesicles.

Representative examples of endogenously expressed GVs native to bacterial or archaeal species are the gas vesicle protein structure produced by the Cyanobacterium Anabaena flos-aquae (Ana GVs) [4], and the Halobacterium Halobacterium salinarum (Halo GVs) [9]. In particular, Ana GVs are cone-tipped cylindrical structures with a diameter of approximately 140 nm and length of up to 2 μm and in particular 200-800 nm or longer. Halo GVs are typically spindle-like structures with a maximal diameter of approximately 250 nm and length of 250-600 nm.

In bacteria or archaea expressing GVs, the genes (herein also gvp genes) encoding for the proteins forming the GVs (herein also GV proteins), are organized in a gas vesicle gene cluster of 8 to 14 different genes depending on the host bacteria or archaea, as will be understood by a skilled person.

The term “Gas Vesicle Genes Cluster” or “GVGC” as described herein indicates a gene cluster encoding a set of GV proteins capable of providing a GV upon expression within a bacterial or archaeal cell Since the ability of expressed GV proteins to assemble in a GV depends on the cell environment where GV proteins are expressed and a same group of gvp genes may or may not form a GV upon expression in a cell, gvp genes provide GVGCs in a cell dependent manner as will be understood by a skilled person (see on point US application 15,663,635 published as US 2018/0030501).

The term “gene cluster” as used herein means a group of two or more genes found within an organism's DNA that encode two or more polypeptides or proteins, which collectively share a generalized function or are genetically regulated together to produce a cellular structure and are often located within a few thousand base pairs of each other. The size of gene clusters can vary significantly, from a few genes to several hundred genes [10]. Portions of the DNA sequence of each gene within a gene cluster are sometimes found to be similar or identical; however, the resulting protein of each gene is distinctive from the resulting protein of another gene within the cluster. Genes found in a gene cluster can be observed near one another on the same chromosome or native plasmid DNA, or on different, but homologous chromosomes. An example of a gene cluster is the Hox gene, which is made up of eight genes and is part of the Homeobox gene family. In the sense of the disclosure, gene clusters as described herein also comprise gas vesicle gene clusters, wherein the expressed proteins thereof together are able to form gas vesicles.

The term “gene” as used herein indicates a polynucleotide encoding for a protein that in some instances can take the form of a unit of genomic DNA within a bacteria, plant, or other organism. The term gene as used herein incudes naturally occurring polynucleotide encoding for a protein as well as engineered polynucleotide whose sequences have been modified from the original sequence for example to optimize expression, e.g. through codon changes (see Examples section) and/or through introduction of modified N- and/or C-terminal modifications, while still maintaining the ability to encode for the protein encoded by the naturally occurring polynucleotide or a or a functional variant thereof.

The term “polynucleotide” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Accordingly, the term polynucleotide includes nucleic acids of any length, and in particular DNA RNA analogs and fragments thereof.

The term “protein” as used herein indicates a polypeptide with a particular secondary and tertiary structure that can interact with another molecule and in particular, with other biomolecules including other proteins, DNA, RNA, lipids, metabolites, hormones, chemokines, and/or small molecules. The term “polypeptide” as used herein indicates an organic linear, circular, or branched polymer composed of two or more amino acid monomers and/or analogs thereof. The term “polypeptide” includes amino acid polymers of any length including full-length proteins and peptides, as well as analogs and fragments thereof. A polypeptide of three or more amino acids is also called a protein oligomer, peptide, or oligopeptide. In particular, the terms “peptide” and “oligopeptide” usually indicate a polypeptide with less than 100 amino acid monomers. In particular, in a protein, the polypeptide provides the primary structure of the protein, wherein the term “primary structure” of a protein refers to the sequence of amino acids in the polypeptide chain covalently linked to form the polypeptide polymer. A protein “sequence” indicates the order of the amino acids that form the primary structure. Covalent bonds between amino acids within the primary structure can include peptide bonds or disulfide bonds, and additional bonds identifiable by a skilled person. Polypeptides in the sense of the present disclosure are usually composed of a linear chain of alpha-amino acid residues covalently linked by peptide bond or a synthetic covalent linkage. The two ends of the linear polypeptide chain encompassing the terminal residues and the adjacent segment are referred to as the carboxyl terminus (C-terminus) and the amino terminus (N-terminus) based on the nature of the free group on each extremity. Unless otherwise indicated, counting of residues in a polypeptide is performed from the N-terminal end (NH₂-group), which is the end where the amino group is not involved in a peptide bond to the C-terminal end (—COOH group) which is the end where a COOH group is not involved in a peptide bond. Proteins and polypeptides can be identified by x-ray crystallography, direct sequencing, immunoprecipitation, and a variety of other methods as understood by a person skilled in the art. Proteins can be provided in vitro or in vivo by several methods identifiable by a skilled person. In some instances where the proteins are synthetic proteins in at least a portion of the polymer two or more amino acid monomers and/or analogs thereof are joined through chemically-mediated condensation of an organic acid (—COOH) and an amine (—NH₂) to form an amide bond or a “peptide” bond.

As used herein the term “amino acid”, “amino acid monomer”, or “amino acid residue” refers to organic compounds composed of amine and carboxylic acid functional groups, along with a side-chain specific to each amino acid. In particular, alpha- or «-amino acid refers to organic compounds composed of amine (—NH₂) and carboxylic acid (—COOH), and a side-chain specific to each amino acid connected to an alpha carbon. Different amino acids have different side chains and have distinctive characteristics, such as charge, polarity, aromaticity, reduction potential, hydrophobicity, and pKa. Amino acids can be covalently linked to form a polymer through peptide bonds by reactions between the amine group of a first amino acid and the carboxylic acid group of a second amino acid. Amino acid in the sense of the disclosure refers to any of the twenty naturally occurring amino acids, non-natural amino acids, and includes both D an L optical isomers.

In embodiments herein described identification of a gene cluster encoding GV proteins naturally expressed in bacteria or archaea as described herein can be performed for example by isolating the GVs from the bacteria or archaea, isolating the protein for the protein shell of the GV and deriving the related amino acidic sequence with methods and techniques identifiable by a skilled person (see e.g. procedures described in [12]). The sequence of the genes encoding for the GV proteins can then be identified by methods and techniques identifiable by a skilled person. For example, gas vesicle gene clusters can also be identified by persons skilled in the art by performing gene sequencing or partial- or whole-genome sequencing of organisms using wet lab and in silico molecular biology techniques known to those skilled in the art. As understood by those skilled in the art, gas vesicle gene clusters can be located on the chromosomal DNA or native plasmid DNA of microorganisms. After performing DNA or cDNA isolation from a microorganism, the polynucleotide sequences or fragments thereof or PCR-amplified fragments thereof can be sequenced using DNA sequencing methods such as Sanger sequencing, DNASeq, RNASeq, whole genome sequencing, and other methods known in the art using commercially available DNA sequencing reagents and equipment, and then the DNA sequences analyzed using computer programs for DNA sequence analysis known to skilled persons.

In some embodiments, identification of a gene cluster encoding for GV proteins [9, 13, 14] can also be performed by screening DNA sequence databases such as GenBank, EMBL, DNA Data Bank of Japan, and others. Gas vesicle gene cluster gene sequences in databases such as those above can be searched using tools such as NCBI Nucleotide BLAST and the like, for gas vesicle gene sequences and homologs thereof, using gene sequence query methods known to those skilled in the art. For example, genes of the gene cluster for the exemplary haloarchael GVs (which have the largest number of different gvp genes) and their predicted function and features are illustrated in Example 26 of related U.S. application Ser. No. 15/613,104, filed on Jun. 2, 2017 which is incorporated herein by reference in its entirety. GV gene clusters can also be identified using a combination of genomic vicinity (e.g. antiSMASH), protein homology and prior GV gene annotation as will be understood by a skilled person.

A GV gene cluster encoding for GV proteins typically comprises Gas Vesicle Assembly (GVA) genes and Gas Vesicle Structural (GVS) genes.

The term Gas Vesicle Structural (GVS) proteins as used herein indicates proteins forming part of a gas-filled protein structure intracellularly expressed by certain bacteria or archaea and can be used as a mechanism to regulate cellular buoyancy in aqueous environments [8]. In particular, GVS shell comprises a GVS identified as gvpA or gvpB (herein also referred to as gvpA/B) and optionally also a GVS identified as gvpC.

In particular, gvpB gene is a gene encoding for gas vesicle structural protein B. gvpB genes is highly homologous to gvpA gene encoding for gas vesicle structural protein A. A gvp A/B is a protein of the GV shell that has a higher than 60% and possibly higher than 70% identity to the following consensus sequence:

(SEQ ID NO: 3)
SSSLAEVLDRILDKGXVIDAWARVSLVGIEILTIEARVVIASVDTYLR

wherein X can be any amino acid. In particular in a gvpA/B of prokaryotes, the consensus sequence of SEQ ID NO: 3 typically forms a conserved secondary structure having an alpha-beta-beta-alpha structural motif formed by portions of the consensus sequence comprising the amino acids LDRILD (SEQ ID NO:4) having an alpha helical structure, RILDKGXVIDAWARVS (SEQ ID NO:5) wherein X can be any amino acid, having a beta strand, beta strand structure, and DTYLR (SEQ ID NO:6) having an alpha helical structure, as will be understood by a skilled person.

As used herein, “homology”, “sequence identity” or “identity” in the context of two nucleic acid or polypeptide sequences makes reference to the nucleotide bases or residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

A functionally equivalent residue of an amino acid used herein typically refers to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

A person skilled in the art would understand that similarity between sequences is typically measured by a process that comprises the steps of aligning the two polypeptide or polynucleotide sequences to form aligned sequences, then detecting the number of matched characters, i.e. characters similar or identical between the two aligned sequences, and calculating the total number of matched characters divided by the total number of aligned characters in each polypeptide or polynucleotide sequence, including gaps. The similarity result is expressed as a percentage of identity.

As used herein, “percentage of sequence identity” means the value determined by comparing two optimally aligned sequences over a comparison window, wherein the portion of the polynucleotide sequence in the comparison window may comprise additions or deletions (gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As used herein, “reference sequence” is a defined sequence used as a basis for sequence comparison. A reference sequence may be a subset or the entirety of a specified sequence; for example, as a segment of a full-length protein or protein fragment. A reference sequence can comprise, for example, a sequence identifiable a database such as GenBank and UniProt and others identifiable to those skilled in the art.

As understood by those skilled in the art, determination of percent identity between any two sequences can be accomplished using a mathematical algorithm. Non-limiting examples of such mathematical algorithms are the algorithm of Myers and Miller [15], the local homology algorithm of Smith et al. [16]; the homology alignment algorithm of Needleman and Wunsch [17]; the search-for-similarity-method of Pearson and Lipman [18]; the algorithm of Karlin and Altschul [19], modified as in Karlin and Altschul [20]. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA [18], and TFASTA in the Wisconsin Genetics Software Package, Version 8 (available from Genetics Computer Group (GCG), 575 Science Drive, Madison, Wis., USA).

Further examples of modern implementations to determine sequence identity include CLUSTAL (2 [Ref. Sievers et al., Mol. Syst. Biol. 7:539, 2011); MUSCLE [Ref. Edgar, Nucleic Acids Res. 32:1792-1797, 2004); MAFFT [Ref. Katoh and Standley, Mol. Biol. Evol. 30:772-780, 2013); and T-Coffee (Notredame et al., J. Mol. Biol. 302:205-217, 2000) for multiple sequence alignments. Additional high-throughput and cloud-based implementations useful for large-scale or metagenomic sequence comparison include NCBI BLAST+ [Ref. Camacho et al., BMC Bioinformatics 10:421, 2009), Diamond (Buchfink et al., Nat. Methods 12:59-60, 2015), MMseqs2 (Steinegger and Söding, Nat. Biotechnol. 35:1026-1028, 2017), and DeepAlign [Ref. Wang et al., Bioinformatics 29:2640-2648, 2013), which employ GPU acceleration or machine-learning-based scoring functions to enhance alignment speed and accuracy. Alignments using these programs can be performed using the default parameters or parameters optimized according to the needs of the user, as understood by those skilled in the art. Alignments using these programs can be performed using the default parameters optimized according to the needs of the user, as understood by those skilled in the art.

Thus, a gvpA/B protein in a prokaryote of interest can be identified for example by isolating GVs from a prokaryote of interest, isolating the protein from the protein shell of the GV and obtaining the amino acid sequence of the isolated protein. In addition or in the alternative to the isolating the GVs and isolating the protein, the method can include obtaining amino acidic sequences of the shell proteins of the GV of the prokaryote of interest from available database. The method further comprises performing a sequence alignment of the obtained amino acidic sequences against the gvpA/B protein consensus sequence of SEQ ID NO:3.

In particular the isolating GVs from a prokaryote of interest can be performed following methods to isolate gas vesicles as described in U.S. application Ser. No. 15/613,104, filed on Jun. 2, 2017. The isolating the protein for the protein shell of the GV and obtaining the related amino acidic sequence can be performed with tandem liquid chromatography mass-spectrometry alone or in combination with obtaining amino acid sequences of the isolated protein with wet lab techniques or from available databases comprising the sequences of the prokaryote of interest as well as additional techniques and approaches identifiable by a skilled person. Obtaining amino acid sequences of GV shell proteins of the prokaryote of interest can be performed by screening available databases of gene and protein sequences identifiable by a skilled person. Performing a sequence alignment of the sequences of the isolated GV proteins or proteins encoded in the genome of a prokaryote of interest can be performed (using Protein BLAST or other alignment algorithms known in the art) against the gvpA/B protein consensus sequence of SEQ ID NO:3. In particular, a sequence alignment can be performed using gvpA/B protein sequences from the closest phylogenetic relative to the prokaryote of interest. Reference is made to Example 1 showing exemplary phylogenetic relationships between gvpA/B proteins of exemplary prokaryotic species.

The optional gvpC gene encodes for a gvpC protein which is a hydrophilic protein of a GV shell, including repetitions of one repeat region flanked by an N-terminal region and a C terminal region. The term “repeat region” or “repeat” as used herein with reference to a protein refers to the minimum sequence that is present within the protein in multiple repetitions along the protein sequence without any gaps. Accordingly, in a gvpC multiple repetitions of a same repeat is flanked by an N-terminal region and a C-terminal region. In a same gvpC, repetitions of a same repeat in the gvpC protein can have different lengths and different sequence identity one with respect to another.

Repeat regions within any given gvpC sequence ‘X’ from organism ‘Y’ can be identified by comparing the related sequence with the sequence of a known gvpC (herein e.g. reference gvpC sequence “Z”). In particular, the comparing can be performed by aligning sequence ‘X’ to the reference gvpC sequence ‘Z’ using a sequence alignment tools such as BLASTP or other sequence alignment tools identifiable by a skilled person at the date of filing of the application upon reading of the present disclosure. In particular, a reference sequence ‘Z’ is chosen from a host that is the closest phylogenetic relative of ‘Y’, from a list of Anabaena flos-aquae, Halobacterium salinarum, Haloferax mediditerranei, Microchaetae diplosiphon and Nostoc sp. The sequence alignment of ‘X’ and ‘Z’ (e.g. a BLASTP) is performed by performing a first alignment of sequence X and sequence Z to identify a beginning and an end of a repeat in ‘X as well as a number of repetition of the identified repeat, in accordance with the known repeats in ‘Z’. The first alignment results in at least one first aligned portion of X with respect to reference sequence Z. The aligning can also comprises performing a second alignment between the at least one first aligned portion of X identified following the first alignment and additional portions of X to identify at least one repeat ‘R1’ in X. Other repeats in ‘X’ (i.e. R2, R3, R4 . . . ) can subsequently be identified with respect to R1. In performing alignment steps sequence are identified as repeat when the sequence shows at least 3 or more of the characteristics described in US application 15,663,635 published as US 2018/0030501 (incorporated herein by reference in its entirety) which also include additional features of gvpC proteins and the related identification.

In a GVGC, the GVS genes are comprised with Gas Vesicle Assembly genes. The Gas Vesicle Assembly genes are genes encoding for GVA proteins. GVA proteins comprise proteins with various putative functions such as nucleators and/or chaperons as well as proteins with an unknown specific function related to the assembly of the GV.

In a prokaryotic cell GVA genes are all the genes within one or more operons comprising at least one of a gvpN and a gvpF excluding any gvpA/B and gvpC gene possibly present within said one or more operons. Therefore GVA genes can be identified by identifying an operon in a prokaryote including at least one of a gvpN and a gvpF excluding any gvpA/B and gvpC gene.

Preferably the one or more operons comprising all the GVA genes of a prokaryote can be identified and detected by detecting a gvpN gene encoding for a GV protein consensus sequence RALXYLQAGYXVHXRGPAGTGKTTLAMHLAXXLXRPVMLIXGDDEFXTSDLIGSESGY XXKKVVDNYIHSVVKVEDELRQNWVDNRLTXACREGFTLVYDEFNRSRPEXNNVLLS VLEEKILXLP (SEQ ID NO: 1) wherein X indicates any amino acid or a sequence of any length having at least 50%, and more preferably 60% or higher, most preferably from 50% to 83% identity.

gvpN genes of various microorganisms have a sequence encoding for a gvpN protein within the consensus SEQ ID NO: 1. In particular, gvpN gene in the sense of the disclosure can be a gene encoding for sequence MTVLTDKRKKGSGAFIQDDETKEVLSRALSYLKSGYSIHFTGPAGGGKTSLARALAKKR KRPVMLMHGNHELNNKDLIGDFTGYTSKKVIDQYVRSVYKKDEQVSENWQDGRLLEA VKNGYTLIYDEFTRSKPATNNIFLSILEEGVLPLYGVKMTDPFVRVHPDFRVIFTSNPAEY AGVYDTQDALLDRLITMFIDYKDIDRETAILTEKTDVEEDEARTIVTLVANVRNRSGDEN SSGLSLRASLMIATLATQQDIPIDGSDEDFQTLCIDILHHPLTKCLDEENAKSKAEKIILEE CKNIDTEEK (SEQ ID NO: 11) or a sequence of any length having at least 30% sequence identity with respect to SEQ ID NO: 11, preferably at least 50%, and more preferably 60% or higher, and gvpF gene in the sense of the disclosure can be a gene encoding for sequence MSETNETGIYIFSAIQTDKDEEFGAVEVEGTKAETFLIRYKDAAMVAAEVPMKIYHPNR QNLLMHQNAVAAIMDKNDTVIPISFGNVFKSKEDVKVLLENLYPQFEKLFPAIKGKIEVG LKVIGKKEWLEKKVNENPELEKVSASVKGKSEAAGYYERIQLGGMAQKMFTSLQKEV KTDVFSPLEEAAEAAKANEPTGETMLLNASFLINREDEAKFDEK VNEAHENWKDKADF HYSGPWPAYNFVNIRLKVEEK (SEQ ID NO:12) or a sequence of any length having at least 20% sequence identity with respect to SEQ ID NO: 12, preferably at least 50%, more preferably 60%, and at least 70% or higher.

The term “operon” as described herein indicates a group of genes arranged in tandem in a prokaryotic genome as will be understood by a skilled person. Operons typically encode proteins participating in a common pathway are organized together as understood by those skilled in the art. Typically, genes of an operon are transcribed together into a single mRNA molecule referred to as polycistronic mRNA. Polycistronic mRNA comprises several open reading frames (ORFs), each of which is translated into a polypeptide. These polypeptides usually have a related function and their coding sequence is grouped and regulated together in a regulatory region, containing a promoter and an operator. Typically, repressor proteins bound to the operator sequence can physically obstruct the RNA polymerase enzyme from binding the promoter, preventing transcription. An example of a prokaryotic operon is the lac operon, which natively regulates transport and metabolism of lactose in E. coli and many other enteric bacteria.

In contrast, eukaryotic and mammalian cells generally do not possess native operons, as transcriptional units are typically monocistronic, resulting in one mRNA per gene. However, operon-like expression strategies can be engineered for use in mammalian systems through the incorporation of self-cleaving peptide sequences (such as 2A peptides derived from picornaviruses), internal ribosome entry sites (IRES elements), or other multicistronic expression constructs. These synthetic or chimeric operons permit coordinated expression of multiple open reading frames from a single promoter while enabling translation of distinct polypeptides in mammalian cells. Accordingly, operon-based or operon-inspired constructs as described herein may be adapted for use in prokaryotic or eukaryotic host cells, including mammalian systems.

In an operon, each ORF typically has its own ribosome binding site (RBS) so that ribosomes simultaneously translate ORFs on the same mRNA. Some operons also exhibit translational coupling, where the translation rates of multiple ORFs within an operon are linked. This can occur when the ribosome remains attached at the end of an ORF and translocates along to the next ORF without the need for a new RBS. Translational coupling is also observed when translation of an ORF affects the accessibility of the next RBS through changes in RNA secondary structure.

In engineered mammalian expression systems, analogous coupling effects can be achieved through use of viral IRES elements or ribosomal skipping sequences (for example, Thosea asigna virus 2A or porcine teschovirus 2A peptides), which mediate efficient multicistronic translation from a single transcript. Such constructs enable the coordinated expression of multiple proteins in mammalian cells in a manner functionally comparable to natural prokaryotic operons, while maintaining proper folding, localization, and stoichiometric balance among the expressed polypeptides.

In some embodiments, a GV cluster comprises one of gvpN or gvpF. In several embodiments GV clusters include both gvpN and gvpF as will be understood by a skilled person. In this connection, reference is made to Example 12 and FIGS. 20 and 21 of related application US application 15,663,635 published as US 2018/0030501 incorporated herein by reference in its entirety, showing exemplary gas vesicle gene clusters operons [1, 2] comprising GVS and GVA genes and related exemplary configuration. In particular, as shown in Example 12 of related application US application 15,663,635 published as US 2018/0030501, typically a native GV gene cluster has GVA genes comprising both gvpN and gvpF genes, even if native GV gene clusters are known having a gvpN gene or a gvpF gene, as understood by skilled persons.

Accordingly, for a certain prokaryote, GVA genes in the sense of the disclosure indicate all the genes that are comprised in the one or more operons having at least one of a gvpN and/or a gvpF herein described and excluding any Gas Vesicle Structural (GVS) genes of the prokaryotes possibly comprised within the one or more operons.

Thus, GVA genes comprised in a gas vesicle gene cluster in a prokaryote can be identified for example by obtaining genome sequence of the prokaryote of interest and performing a sequence alignment of the protein sequences encoded in the genome of the prokaryote of interest against a gvpN protein sequence and/or a gvpF protein sequence.

In particular, obtaining the genome sequence of the prokaryote of interest, can be performed either using wet lab techniques identifiable by a skilled person upon reading of the present disclosure, or obtained from databases of gene and protein sequences also identifiable by a skilled person upon reading of the present disclosure. Performing a sequence alignment of the protein sequences encoded in the genome of the prokaryote of interest can per performed using Protein BLAST or other alignment algorithms identifiable by a skilled person. Exemplary gvpN protein sequence and/or a gvpF protein sequence, that can be used in performing the alignment are sequences SEQ ID NO:11 and/or SEQ ID NO:12. In particular, a sequence alignment can be performed using gvpN and/or gvpF protein sequences from the closest phylogenetic relative to the prokaryote of interest. Reference is made to Example 2 showing exemplary phylogenetic relationships between gvpF and gvpN proteins of exemplary prokaryotic species. Accordingly, one or more operons that comprise the gvpN and/or gvpF genes can be identified, and any other gvps within the one or more operons can also be identified, wherein the other gvps are comprised in ORFs within the one or more operons, excluding any ORFs encoding gvpA/B or gvpC genes comprised in the one or more operons of the GV gene cluster.

Accordingly, GVA genes can also be identified based on the configuration of operon and Gene Clusters identified through homology (see e.g. Example 1), phylogenesis (see e.g. Example 2) also using the gvpA/B, gvpN and/or gvpF consensus of SEQ ID Nos: 1, 3, and 11-12 herein provided, preferably gvpA/B consensus of SEQ ID NO: 3 and gvpN consensus of SEQ ID NO: 1. Reference is also made in this connection to the indication of Example 3 reporting exemplary GVGC configurations of naturally occurring Gas Vesicle gene clusters identified with method herein described and additional methods identifiable by a skilled person.

GVS genes of a GVGC of the disclosure, identified with methods herein indicated, typically comprise gvpA or gvpB which have similar sequences and are equivalent in their purpose and optionally gvpC. Exemplary sequences for gvpA and gvpB genes of GV gene clusters in the sense of the disclosure, which can also be used to identify additional GVS and GVGC through homology and alignment in addition to the use of the consensus sequence SEQ ID NO: 3, are reported in Example 4.

GVA genes of a GVGC of the disclosure, identified with methods herein indicated, typically comprise proteins identified as gvpF, gvpG, gvpL, gvpS, gvpK, gvpJ, and gvpU. GVA genes and proteins can also comprise gvpR and gvpT (see e.g. B. megaterium GVA) gvpV, gvpW (se Anaboena flos aque and Serratia GVA) and/or gvp X, gvp Y and gvp Z (see e.g. Serratiai GVA. Preferably GVGC of the disclosure further comprise gvpN which result in a more robust detection with many detection methods herein described. Exemplary sequences for GVA genes of GV gene clusters in the sense of the disclosure which can also be used to identify additional GVAs and GVGC through homology and alignment are reported in Example 4.

In GVGC herein described co-expression of the GVS genes and the GVA genes in connection with regulatory sequence capable of operating in a host cell are configured to provide a GV type, with a different GVGC typically resulting in a different GV type.

The wording “GV type” in the sense of the disclosure indicates a gas vesicle having dimensions and shape resulting in distinctive mechanical, acoustic, surface and/or magnetic properties as will be understood by a skilled person upon reading of the present disclosure. In particular, a skilled person will understand that different shapes and dimensions will result in different properties in view of the indications in provided in U.S. Applicant Ser. No. 15/613,104 published as US2018/0028693 and U.S. Ser. No. 15/663,600 published as US2018/0038922 and additional indications identifiable by a skilled person Typically, larger volume results in stronger per-particle scattering, smaller diameter generally results in higher collapse pressure after removal of gvpC, and different dimensions result in different ratios of T2/T2* relaxivity per volume-averaged magnetic susceptibility ([21]).

In addition, a “GV type” can further encompass variations determined by the structure, truncation, or modification of the outer scaffold protein GvpC, as will be understood by a skilled person. In particular, GV types can comprise truncated or partially deleted GvpC (for example, ΔN-, ΔC-, or ΔN&C-terminus variants) and GV types lacking GvpC entirely (ΔGvpC) which can exhibit reduced collapse pressures and enhanced nonlinear acoustic responses relative to wild-type GVs [Ref. U.S. Pat. No. 10,493,172]. Similarly, GVs with weakened or cleaved GvpC coatings facilitate acoustic collapse and cavitation under therapeutic ultrasound conditions, representing a distinct functional GV type [Ref. U.S. Pat. No. 11,446,523] and mammalian-expressible GV types can include full-length, truncated, or omitted GvpC forms, each conferring specific acoustic and magnetic profiles in eukaryotic expression environments [Ref U.S. Pat. No. 11,761,008].Additionally, modification of the gvpC can also result in the protease-sensitive GVPC constructs which can be included in GV type bearing cleavable sequences, wherein protease-mediated cleavage dynamically alters the mechanical reinforcement of the GV wall and thereby changes the collapse pressure and nonlinear imaging response [Ref. U.S. application Ser. No. 17/006,591].

Accordingly, the “GV type” in the sense of the present disclosure includes any gas vesicle structure whose mechanical or acoustic characteristics are determined not only by vesicle size and geometry but also by the configuration of GvpC—whether full-length, truncated, omitted, or engineered to be protease-cleavable—and combinations thereof, as will be understood by a skilled person upon reading of the present disclosure.

In addition, the GV type encompasses vesicles whose structural or functional properties are modified post-translationally or post-assembly, including chemical or enzymatic alteration of GvpC or other shell proteins. Such modifications can include stripping or partial removal of GvpC from the vesicle surface, chemical crosslinking, PEGylation, acylation, or conjugation of small molecules, peptides, or polymers to the GV shell. These treatments, as described for example in U.S. Pat. Nos. 10,493,172 B2, 11,446,523 B2, and 11,761,008 B2, can be used to modulate GV stability, surface charge, hydrophobicity, or acoustic non-linearity, thereby providing additional GV types distinguished by their post-translational or chemical modification state. Accordingly, GV type in the sense of the disclosure encompasses native, genetically engineered, and chemically or biochemically modified gas vesicles whose collapse pressure, resonance behavior, or magnetic or optical response is determined by both protein composition and post-synthetic modification of the GV shell as will be understood by a skilled person.

Accordingly, in embodiments herein described, GVGC can be selected based on desired properties of the corresponding GV type. In particular, to this extent, a skilled person can use naturally occurring GVGC, can provide engineered GVGC wherein some of the naturally occurring gvp genes are omitted, and/or can provide hybrid GVGC in which GVAs and GVS genes of naturally occurring GVGCs are combined to provide GV types having the shape and dimensions resulting in the desired properties.

The term “hybrid gene cluster” or “hybrid cluster” as used herein indicates a cluster comprising at least two genes native to different species and resulting in a cluster not natively in any organisms. Typically, a hybrid gene cluster comprises a subset of gas vesicle genes native to a first bacterial species and another subsets of gas vesicle genes native to one or more bacterial species, with at least one of the one or more bacterial species different from the first bacterial specie Accordingly, a hybrid GV gene clusters includes a combination of GV genes which is not native in any naturally occurring prokaryotes.

In particular, identification of a desired GVGC for a target cell and therefore of the ability of the corresponding gvp genes combination to result in production of functional GV proteins capable of assembling in a GV thus providing a corresponding detectable GV type can be performed through a testing method also directed to verify detectability of the GV by a detection method of choice. The testing method can be performed in the target cell where detection of the GV type is desired or in testing cells having a cell environment equivalent to the cell environment of the target cell in terms of expression of GV genes and GV formation and thus provide a model to verify ability of the gvp genes to provide a GVGC for the target cells. In the method to identify a desired GVGC the introducing can be performed using engineered polynucleotide constructs contacted with the target cell or testing cell for a time and under conditions to allow expression of the GVGC and formation of the GV type (e.g. using the methods described in US application 15,663,635 published as US 2018/0030501 incorporated herein by reference). The method further comprises detecting formation of a gas vesicle in the target cell or testing cell following the introducing with a pre-set method of detection. Preset methods of detection can be directed to detect acoustic and/or magnetic properties that are of interest in desired applications of the corresponding GV type. Preferably the testing can be performed in a target cell or testing cell, that have been modified, either chemically or genetically, to have the same cellular turgor pressure as mammalian cells according to methods identifiable by a skilled person.

Experiments performed with GVGC herein described (e.g., in the Examples section) provide proof of principle that a permissive mammalian cell line (e.g., HEK293T) known to be highly transducible is an effective initial model for assessing the ability of a GVGC to correctly assemble and produce a signal in other, less-permissive or hard-to-transduce mammalian cells (e.g., primary T cells). Accordingly, detecting expression and ultrasound signal from a candidate GVGC in a model mammalian cell line using a pre-set method is indicative of the ability of the corresponding GV proteins to form a functional GV type in other mammalian cells, including less permissive or hard to transduce types like clinically relevant primary T cells. Therefore, a method for optimizing gas vesicle expression in a target mammalian cell type comprises: (a) providing the multi-vector gas vesicle expression system components; (b) transducing aliquots of the target cells with the components using systematically varied total viral titers and systematically varied relative ratios of the vector components; and (c) measuring gas vesicle expression levels and/or functional ultrasound signal under standard conditions for each aliquot to identify the titer and ratio combination that yields maximal functional output in the target cell type.

In exemplary embodiments where a GV type is to be used in differential ultrasound imaging or image-subtracted ultrasound, the pre-set method of detection can comprise imaging with ultrasound a target site comprising the cell following the introduction of the GVGC, applying acoustic pressure to the target site at a pressure expected to collapse the GVs and then imaging the target site with ultrasound again, and the difference of the images (before and after collapse) shows if collapsing GVs (having a collapse threshold below the acoustic pressure) were present at the target site.

In exemplary embodiments where a GV type is to be used in MRI (magnetic resonance imaging), imaging, the pre-set method of detection can comprise imaging with MRI a target site comprising the cell following the introduction of the GVGC, applying hydrostatic pressure to the target site at a pressure expected to collapse the GVs. The target site is then imaged with MRI again, and the difference of the images (before and after collapse) shows if collapsing GVs (having a collapse threshold below the hydrostatic pressure) were present at the target site. In further embodiments, detection of the GV type can also include measurement of an increase in magnetic susceptibility or non-linearity of the signal, as the collapse or structural modulation of the GV shell—particularly in variants having truncated or cleavable GvpC as described in U.S. Pat. No. 10,493,172 B2 and U.S. Pat. No. 11,761,008 B2—produces a quantifiable change in MRI contrast. Multiplexed detection of multiple GV types, distinguished by their differing collapse pressures or relaxation behavior, can further be employed to enable multi-parametric or multi-target MRI imaging of distinct GV populations within the same field of view.

In exemplary embodiments where a GV type is to be used in BURST (burst ultrasound reconstruction with signal templates) imaging described herein and in U.S. application Ser. No. 16/736,581 filed on Jan. 7, 2020 and herein incorporated by reference in its entirety, the pre-set method of detection can comprise imaging with ultrasound a target site comprising the cell following the introduction of the GVGC, over successive frames, at a peak positive pressure (PPP) well below the expected collapse threshold pressure for the GVs. While the frames are being taken, increasing the PPP step-wise to a value over the expected collapse threshold pressure for at least 9 half-cycles. Frames from before, during, and after the application of the increased pressure undergo template mixing to detect a BURST signal from the collapsing GVs, if present. Additionally, GV detection can include monitoring increases in acoustic non-linearity, such as harmonic or subharmonic signal generation, characteristic of GVs with truncated, weakened, or protease-cleavable GvpC coatings as disclosed in U.S. Pat. Nos. 10,493,172 B2, 11,446,523 B2, and 11,761,008 B2. These non-linear responses can be analyzed to discriminate GV types according to their distinct collapse thresholds, enabling multiplexed ultrasound imaging in which multiple GV populations are simultaneously resolved by their unique non-linear pressure-response profiles

Additional methods of detection such as Transmission Electron Microscopy (TEM) and optical scattering, optical phase detection, and xenon hyperCEST MRI can be used. Further exemplary embodiments can employ detection of GV types based on acoustic or magnetic non-linearity, for instance by analyzing harmonic, subharmonic, or higher-order components of the ultrasound or magnetic resonance signal as described in U.S. Pat. Nos. 10,493,172 B2, 11,446,523 B2, and 11,761,008 B2. Such nonlinear detection enables identification of GVs that differ by GvpC truncation, deletion, or protease sensitivity, which manifest distinct collapse thresholds or harmonic response profiles. In some embodiments, multiplexed detection of multiple GV types can be achieved by combining signals from GVs having different non-linear characteristics or collapse pressures, allowing simultaneous imaging or sensing of multiple biological events or reporter populations within a single target site. Additional optical and magnetic resonance approaches—including phase-contrast microscopy, dynamic light scattering, and susceptibility-weighted MRI—can likewise be used to distinguish GV types based on changes in scattering intensity, relaxation rates, or susceptibility following GV collapse or modification.

An exemplary method of detection of a functional GVGC in the sense of the disclosure performed in E. Coli is reported in Example 5 of the present disclosure. Additional methods to be performed other prokaryotic cells and/or mammalian cells using the GVES of the disclosure can be identified by a skilled person upon reading of the present disclosure.

Several detectable GVGC with one or more detection method of interests have been identified and can be used for production of GV types in various cells through various genetically engineered constructs as will be understood by a skilled person upon reading of the present disclosure and US application 15,663,635 published as US 2018/0030501 herein incorporated by reference in its entirety.

Exemplary GVGCs comprise native or engineered clusters that give rise to gas vesicles exhibiting distinct collapse pressures, acoustic non-linearities, or magnetic susceptibilities, thereby providing characteristic signatures under ultrasound or MRI detection as described in U.S. Pat. Nos. 10,493,172 B2, 11,446,523 B2, and 11,761,008 B2. In particular, GVGCs comprising gvpC variants that are truncated, partially deleted, or engineered to include cleavable or protease-sensitive domains yield GV types with modulated mechanical stability and enhanced non-linear acoustic responses, enabling multiplexed imaging or sensing of different GV populations within a single specimen. Such GVGCs can be incorporated into expression systems suitable for prokaryotic, eukaryotic, or mammalian hosts, under the control of appropriate promoters and regulatory elements, to produce reporter cells or organisms optimized for one or more of the aforementioned detection modalities.

In some embodiments described herein GVGC, the instant disclosure can be naturally occurring combination of gvp genes which can have a naturally occurring sequence or a sequence modified to optimize the expression in the cell where detection is to be performed. For example GVGC clusters of the instant disclosure comprise a GVGC of B. megaterium formed by the gvpA or gvpB genes, gvpR, gvpN gvpF, gvpG, gvpL gvpS, gvpK, gvpJ, gvpT, gvpU of B. megaterium, or the GVGC of Anaboena flos aquae formed by the gvpA or gvpB genes of Anaboena flos aquae (see e.g. the sequences in Table 6 of Example 4) and the GVA gvpC, gvpN, gvpJ, gvpK, gvpF, gvpG, gvpV, gvpW of Anaboena flos aquae (see e.g. sequences in Table 10 of Example 4).

The gvp genes in one or more genes of the GVGC cluster of the present disclosure can have a naturally occurring sequence or a sequence modified to optimize the expression in the cell where detection is to be performed. For example a B. megaterium GVGC can have a gvpA or gvpB genes having the sequences in Table 6 of Example 4, and/or any one of the gvpR, gvpN gvpF, gvpG, gvpL gvpS, gvpK, gvpJ, gvpT, gvpU genes having the sequences in Table 8 of Example 4. Similarly, an Anaboena Flos Aquae GVGC can have the gvpA or gvpB genes having the sequences reported in Table 6 of Example 4 and/or any one of the gvpC, gvpN, gvpJ, gvpK, gvpF, gvpG, gvpV, gvpW having the. sequences reported in Table 10 of Example 4.

In some embodiments, described herein, GVGC of the instant disclosure can be modified version of naturally occurring GV gene clusters. An example is provided by the. GVGC of B. megaterium comprising gvpB, gvpR, gvpN gvpF, gvpG, gvpL gvpS, gvpK, gvpJ, gvpT, gvpU wherein the gvpR and gvpT genes of the naturally occurring GVGC from B. megaterium have been omitted (see e.g. the sequences reported in Example 6 and Table 9 of the instant disclosure). Another example is provided by GV gene clusters comprising gvpA, Ana-gvpC gvpN, gpvJ, gvpK, gvpF, gvpG, gvpW, and gvpV from Anabaena flos-aquae or GV gene clusters comprising gvpA+gvpN, gpvJ, gvpK, gvpF, gvpG, gvpW, gvpV from Anabaena flos-aquae (see Anabaena flos-aquae genes in Table 4 and Table 10 of Example 4 of the present disclosure).

In other embodiments described herein, GVGC of the instant disclosure can be a hybrid GV gene cluster in a Gas Vesicle expression system of the disclosure, can comprise a combination of genes from A. flos-aquae (herein also Ana-gvp) and genes from B. megaterium (herein also Mega-gvp). In particular, in exemplary embodiments, the hybrid GV gene cluster can comprise B. megaterium GVA assembly genes gvpR, gvpN, gvpF, gvpG, gvpL, gvpS, gvpK, gvpJ, gvpT and gvpU and further comprise structural gvpA gene from Anabaena flos-aquae. In some of those embodiments, the hybrid GV gene cluster can comprise gvpA, gvpC from Anabaena flos-aquae and GVA genes from B. megaterium possibly excluding gvpR and/or gvpT. In some of those embodiments, the hybrid GV gene cluster can comprise Ana-gvpA and mega GVA genes possibly excluding gvpR and/or gvpT. In some embodiments GVGC of the instant disclosure can include gvpA, gvpC, gvpN from Anabaena flos-aquae and GVA genes from B. megaterium, as well as other combinations identifiable by a skilled person upon reading of the present disclosure.

In some embodiments herein described, a GVGC comprising gvp genes A/B, C and N (gvpA/B, gvpC, gvpN genes) from a same or different prokaryote. Preferably the GVGC comprises a gvpN gene as presence of gvpN protein results in an increased detectability of the related GV type.

For example, in one exemplary embodiment, all the gvp genes B, N, F, G, L, S, K, J and U are from B. megaterium. GVs from B. megaterium are typically cone-tipped cylindrical structures with a diameter of approximately 73 nm and length of 100-600 nm, encoded by a cluster of eleven or fourteen different genes, including the primary structural protein, gvpB, and several putative minor components and putative chaperones [22, 23] as would be understood by a person skilled in the art.

In some embodiments, some of the set of nine gvp genes can be from Bacillus megaterium and the rest genes are from Anabaena flos-aquae such as the GVGC comprising Ana-A, Ana-C, Ana-N, mega: gvpF, gvpG, gvpL, gvpS, gvpK, gvpJ, gvpT and gvpU with/without gvpR and gvpT, and additional examples identifiable by a skilled person upon reading of the present disclosure (see Example 4 and Example 5 of the present disclosure).

In embodiments herein described, the sequences of at least one gvp gene can be modified with respect to the natural occurring sequence to improve the related expression (e.g. to be codon optimized) and/or the inclusion in the GVES of the disclosure (e.g. by modification of the N- and/or C-terminal portions to allow the use of linker or other elements to be included in a cassette or construct of the disclosure).

In some embodiments, the GVGC can comprise Serratia gvp genes as Serratia GVs can express functional GV proteins in E. coli, as reported in literature ([24] [25]).

GVES and related constructs have been herein provided based on the surprising finding that a naturally occurring, or engineered functional GVGC can be transfected and expressed in mammalian cells with a construct including differentially expressed gene modules stoichiometrically configured to allow expression in the mammalian cell of GV proteins encoded by the GVGC resulting in formation of a corresponding GV type in the mammalian cell with an increased yield in GV type obtained.

The term “mammalian cell” refers to cells from a mammal tissue comprising cell within a mammal host and cell isolated from and expanded in culture for use as therapeutic and research tools. Exemplary mammalian cells that can express GVES of the disclosure are primary cells (cells that are directly harvested from an animal and genetically engineered with GVs. Exemplary mammalian cell culture that can be genetically engineered with GV constructs described herein configured to allow expression of GVs comprise HEK 293T, CHO-K1 cells, HEK293, CHO-K1, N2A cells, HeLa, Jurkat, NIH3T3, and other identifiable by those skilled in the art.

Additional examples of mammalian cells suitable to be used in connection with Stoichiometric GVES of the disclosure include induced pluripotent stem cells (iPSCs), embryonic stem cells, mesenchymal stem cells, neuronal and glial cells, hepatocytes, cardiomyocytes, fibroblasts, macrophages, T lymphocytes, and natural killer (NK) cells, as well as immortalized derivatives thereof. In the context of the present disclosure, mammalian cells expressing GV expression systems (GVES) or related constructs (GVPC, GVRMC, GVRGC) may provide GVs exhibiting cell-type-specific assembly or post-translational processing of GV proteins, including glycosylation, phosphorylation, proteolytic cleavage, or partial stripping of GvpC or related structural proteins, as described for example in U.S. Pat. Nos. 10,955,496 B2 and 11,761,008 B2. These cell-specific biochemical environments can modulate GV stability, collapse pressure, and acoustic non-linearity, thereby giving rise to distinct GV types within mammalian expression systems. Accordingly, “mammalian cell” in the sense of the present disclosure includes any genetically engineered cell derived from a mammalian lineage that is capable of expressing, assembling, modifying, or maintaining GVs or GV-like nanostructures as described herein, whether in vitro, in vivo, or ex vivo.

As used herein, the term “differentially expressed gene module” refers to a set or subset of two or more coding sequences within a single gene expression cassette—such as a polycistronic or multicistronic construct—that are expressed at distinct relative levels or stoichiometric ratios as a result of one or more internal regulatory elements. A differentially expressed gene module thus enables coordinated yet non-equivalent expression of multiple proteins from a common transcript or promoter while maintaining functional assembly of the encoded complex or structure.

Differential expression within the module may be governed by transcriptional, translational, or post-translational regulatory features, including but not limited to: (i) promoters or sub-promoters of differing strengths; (ii) internal ribosome entry sites (IRES) with variable initiation efficiency; (iii) ribosome-binding sites or Kozak sequences of differing translation initiation potential; (iv) separation elements such as viral 2A peptides or protease-cleavable linkers with differential cleavage kinetics; (v) RNA structural elements or codon-usage biases affecting mRNA stability or translation rate; and (vi) post-translational processing motifs or degradation tags altering protein half-life.

In certain embodiments, each differentially expressed gene module comprises a subset of gvp genes—for example, gvpA/B, gvpC, and one or more accessory gvp genes-whose relative expression ratios are optimized to yield a gas vesicle type with defined size, shell composition, and acoustic response. The internal regulatory architecture of the module can thus be designed to ensure that stoichiometric balance among Gvp proteins is maintained within a single polycistronic transcript, while still permitting differential expression amplitudes suitable for proper vesicle assembly.

In exemplary embodiments, differentially expressed gene modules are designed using computational modeling and synthetic-biology design tools (for example, in silico prediction of translation efficiency or ribosome profiling data) to achieve target expression ratios and dynamic ranges. These ratios can be predetermined experimentally or through predictive modeling to optimize vesicle morphology, collapse threshold, or nonlinear acoustic contrast.

Differentially expressed gene modules can be used individually or in combination within a multicassette or multigenic construct, enabling hierarchical or multi-layer control of expression. For instance, one module may encode structural GV proteins expressed at high levels, while a second module encodes regulatory or accessory GV proteins expressed at reduced or inducible levels. Such modular design allows precise tuning of vesicle assembly kinetics, mechanical integrity, and imaging properties in mammalian, prokaryotic, or cell-free expression systems.

Accordingly, the term “differentially expressed gene module” encompasses any operably linked group of genes within a cassette whose expression levels are intentionally varied relative to one another by engineered regulatory architecture. These modules permit rational control of protein stoichiometry and post-transcriptional dynamics, thereby enabling predictable customization of GV types and acoustic or magnetic phenotypes across host systems.

In some embodiments, a differentially expressed gene module can be included as part of a monocistronic or a polycistronic gene expression cassette, as will be understood by a skilled person

The term “gene cassette” as used herein indicated a mobile genetic element that contains at least one gene and a recombination site. Accordingly, a gene cassette can contain a single gene (herein a “monocistronic cassette”) or multiple genes (herein a “polycistronic cassette”), which may be organized in an operon structure. A gene cassette can be transferred from one DNA sequence (usually on a vector) to another by ‘cutting’ the fragment out using restriction enzymes or transposase, cripr, viral and/or recombinase enzymes and other nucleases and ‘pasting’ it back into the new context or other molecular biology and cloning techniques (e.g. pcr, CRISPR, TALENs, ZFN). Gene cassettes can move around within an organism's genome or be transferred to another organism in the environment via horizontal gene transfer.

A “gene expression cassette” is a gene cassette comprising regulatory sequence to be expressed by a transfected cell. Following transformation, the expression cassette directs the cell's machinery to make RNA and proteins. Some expression cassettes are designed for modular cloning of protein-encoding sequences so that the same cassette can easily be altered to make different proteins. An expression cassette is composed of one or more genes and the sequences controlling their expression. An expression cassette typically comprises at least three components: a promoter sequence, an open reading frame, and a 3′ untranslated region that, in eukaryotes, usually contains a polyadenylation site. An expression cassette can be formed by manipulable fragment of DNA carrying, and capable of expressing, one or more genes of interest optionally located between one or more sets of restriction sites Gene expression cassettes as used herein typically comprise further regulatory sequences additional to the prompter to regulated the expression of the gene or genes within the open reading frame herein also indicated as coding region of the cassette. As used herein, an expression cassette comprising a single gene or open reading frame is a “monocistronic expression cassette,” and an expression cassette comprising two or more genes or open reading frames (e.g., linked by 2A peptides or IRES elements) is a “polycistronic expression cassette.”

In particular, in embodiments of the GVES herein described, the gene expression cassettes of the system comprise one or more gvp genes under control of regulatory sequence capable of operating in the mammalian host and are thus configured to provide a GV type in the mammalian cell.

The term “regulatory sequence” or “regulatory regions” as described herein indicate a segment of a nucleic acid molecule which is capable of increasing or decreasing transcription or translation of a gene within an organism either in vitro or in vivo. In particular, coding regions of the GV genes herein described comprise one or more protein coding regions which when transcribed and translated produce a polypeptide. Regulatory regions of a gene herein described comprise promoters, transcription factor binding sites, operators, activator binding sites, repressor binding sites, enhancers, protein-protein binding domains, RNA binding domains, DNA binding domains, silencers, insulators and additional regulatory regions that can alter gene expression in response to developmental and/or external stimuli as will be recognized by a person skilled in the art

The term “operative connection” as used herein indicate an arrangement of elements in a combination enabling production of an appropriate effect. With respect to genes and regulatory sequences an operative connection indicates a configuration of the genes with respect to the regulatory sequence allowing the regulatory sequences to directly or indirectly increase or decrease transcription or translation of the genes.

Regulatory sequences used in gene expression cassettes herein described identified herein also as mammalian regulatory regions are configured to operate in a mammalian cell.

Exemplary regulatory regions capable of operating in mammalian cells comprise promoters, enhancers, silencers, terminators, regulators, operators, ribosome binding/entry sites, and riboswitches, among others known in the art. Regulatory regions capable of operating in a mammalian host can be selected by a skilled person following selection of the mammalian host of interest. Exemplary constitutive and inducible mammalian promoters and operators suitable for regulating expression of GVs in a mammalian host comprise and others identifiable by those skilled in the art and described herein.

Mammalian regulatory regions comprised in a gene expression cassette herein described, typically comprise a mammalian promoter, 5′UTR regions, 3′UTR regions, and a terminator as will be understood by a skilled person. Additional regulatory features, such as upstream open reading frames (uORFs), post-transcriptional response elements, or RNA-stabilizing motifs, may be included to modulate expression kinetics or mRNA stability.

A “mammalian promoter” in the sense of the disclosure suitable for gene expression in a mammalian cell is a region of DNA that leads to initiation of transcription of a particular gene. Exemplary are typically located on a same strand and upstream on a DNA sequence (towards the 5′ region of the sense strand), adjacent to the transcription start site of the genes whose transcription they initiate. In mammalian cells organisms, promoters typically comprise the eukaryotic TATA (SEQ ID NO:13) box. Promoters are located near the transcription start sties of genes, upstream on the DNA. Promoters can typically be about 100-1000 base pairs long. In particular promoters that can be used in gene expression cassette herein described can be a constitutive promoter or a conditional promoter.

The term “conditional promoter” refers to a promoter with activity regulatable or controlled by endogenous transcription factors or exogenous inputs such as chemical, or thermal inducers or optical induction. Examples of mammalian constitutive promoters include inducible promoters based on exogenous agents such as TET (tetracycline-response elements, TET-ON/TET-OFF), Lac, dCas-transactivator, Zinc-finger-TF, TALENs-ZF Gal4-uas, synNotch and inducible promoters based on endogenous signals TNF-alpha, cFOS and others identifiable to a skilled person. Conditional promoters enable spatial and temporal control of GV expression and can be used to link vesicle formation to specific biological events or therapeutic triggers.

The term “constitutive promoter” refers to an unregulated promoter that allows for continual transcription of its associated genes. Exemplary mammalian constitutive promoters that can be used for expression in mammalian cell include CMV from human cytomegalovirus, EF1a from human elongation factor 1 alpha, SV40 from the simian vacuolating virus 40, PGK1 from phosphoglycerate kinase gene, Ubc from human ubiquitin C gene, human beta actin, CAAG, SynI and others identifiable to those skilled in the art. Promoter strength and expression kinetics can be modulated by incorporating promoter variants, tandem promoter arrays, or synthetic hybrid promoters.

The wording “5′UTR region” refers to the region upstream from the initiation codon as will be understood by a person of ordinary skill in the art and is therefore outside the coding region of the cassette. The 5′UTR region can contain a Kozak sequence. The Kozak sequence used herein refers to a nucleic acid motif that functions as the protein translation initiation site in most eukaryotic mRNA transcripts as will be understood by a person skilled in the art. The Kozak sequence locates approximately 6 nucleotide sequence upstream of the ATG start codon. Exemplary Kozak sequence include GCCACCATG (SEQ ID NO: 475), TTCACCATG (SEQ ID NO: 476), (CCC) TTCACCATG (SEQ ID NO: 477) consensus sequence XXX [A/G] XXATG (SEQ ID NO: 478) wherein X indicates any nucleotide, and additional sequences identifiable by a skilled person.

The “3′UTR region” refers to an untranslated region that immediately follows the translation termination codon and is therefore outside the coding region of the cassette. 3′UTR region often contains regulatory regions that post-transcriptionally influence gene expression. Regulatory regions within the 3′UTR can influence polyadenylation, translation efficiency, localization, and stability of the mRNA as will be understood by a person skilled in the art. In some embodiments, the 3′UTR contains silencer regions which are configured to bind to repressor proteins and inhibit the expression of the mRNA. The 5′UTR can further contain regulatory motifs such as upstream ORFs, stem-loop structures, or aptamers that modulate translation efficiency and responsiveness to cellular signals. Engineering of the 3′UTR region can be used to fine-tune transcript half-life, enabling modulation of GV protein levels in response to environmental or developmental cues.

A “terminator” as used herein indicates a sequence-based element that defines the end of a transcriptional unit and initiates the process of releasing the synthesized mRNA. Exemplary mammalian terminators include polyadenylation sites. A “polyadenylation site” indicates an element target by the polyadenylation enzymes such as CPSF and typically comprises the sequence AAUAAA (SEQ ID NO: 14) on the RNA. Polyadenylation sites will result in cleavage of the construct 10-30 nucleotides downstream the site, and addition of a poly(A) tail located at the end of 3′UTR as will be understood by a person skilled in the art. In gene expression cassette the poly(A) site can include SV40 polyadenylation element, hGH poly(A) signal, and other poly(A) signal that have the canonical AAUAAA (SEQ ID NO: 14) region as will be understood by a skilled person.

In some embodiments, a gene expression cassette can include additional mammalian regulatory regions configured to increase or decrease the expression of the GV coding regions of the cassette, as will also be understood by a skilled person.

Exemplary mammalian regulatory sequences increasing transcription of the operatively linked gene comprise enhancers that can be located more distally from the transcription start site compared to promoters, and either upstream or downstream from the regulated genes, as understood by those skilled in the art. Enhancers are typically short (50-1500 bp) regions of DNA that can be bound by transcriptional activators to increase transcription of a particular gene. Typically, enhancers can be located up to 1 Mbp away from the gene, upstream or downstream from the start site. An exemplary additional mammalian regulatory regions directed to enhance the expression levels of the GV genes, include Woodchuck Hepatitis Virus Posttranscriptional Regulatory Element (WPRE) placed downstream of the genes between GV gene and the poly(A) tail. The WPRE and WPRE-like (e.g. RE of Hepatitis B virus (HPRE)) element is known to increase transgene expression from a variety of viral vectors. Additional, examples include viral enhancers (SV40, CMV), synthetic enhancer arrays, and post-transcriptional regulatory elements such as the Woodchuck Hepatitis Virus Posttranscriptional Regulatory Element (WPRE) or its analogs (e.g., HPRE), which stabilize transcripts and increase transgene expression in mammalian hosts.

Exemplary mammalian regulatory sequences decreasing transcription of the operatively linked gene comprise RNAi/miRNA/shRNA sites that can be located upstream or downstream of the GV genes to control mRNA translation or degradation. For example, by binding to specific sites within the 3′UTR, miRNAs can decrease gene expression of various mRNAs by either inhibiting translation or directly causing degradation of the transcript.

Additional mammalian regulatory sequences that can be included in a gene expression cassette include post transcriptional regulatory sequences such as riboswitches typically present in eukaryotic untranslated regions (UTRs) of encoded RNAs. These sequences are configured to switch between alternative secondary structures in the RNA depending on the concentration of key metabolites. The secondary structures then either block or reveal other regulatory sequence regions such as RNA binding proteins. A further examples of additional post transcriptional regulatory sequences regulatory sequences comprise aptazymes fusions composed of an aptamer domain and a self-cleaving ribozyme which can be used for conditional gene expression to control mRNA levels with small molecules (e.g. tetracycline).

In general, selection of promoter and other regulatory sequences to be included in expression polynucleotidic constructs comprised in GVES of the present disclosure can be performed by one or more of the following: detecting functionality of a promoter and/or additional regulatory sequence in the host cells, selecting promoters and/or additional regulatory sequences known to be functional in the host cells; detecting the strength of the promoters and/or additional regulatory sequences in connection with protein production and/or selecting promoter and/or additional regulatory sequences of known strength; and selecting inducible promoters and/or additional regulatory sequence to control GV expression.

Mammalian regulatory sequences can be provided in any configuration which is directed to provide a desired expression of the GV protein in the coding regions. For example, a gene expression cassette can an end of UTR with polyA site only, or can be with WPRE and polyA site, or it can be with WPRE only. A combination of WPRE and polyA tail is expected to result in highest expression (highest copy of translated protein). Additional configuration can be identified by a skilled person.

In embodiments of the GVES herein described GV genes other than gvpA/B can be provided in a single gene expression cassette in various combinations and in any order to the extent that when the cassette comprises two or more gvp genes other than gvpA/B, the two or more gvp genes are configured to have each GV gene linked to another by a separation element.

A “separation element” as used herein refers to an element that can be placed between two adjacent coding genes allowing for a separate transcription or translation of the two adjacent coding genes. Separation elements are used in the present disclosure to enable coordinated expression of multiple gas vesicle (GV) proteins from a single transcript while ensuring that each protein is produced as a discrete polypeptide capable of proper folding and assembly into a functional GV type.

In some embodiments, a separation element can be an internal ribosome entry site (“IRES”). An internal ribosome entry site (IRES) used herein refers to an element that allows for translation initiation in a cap-independent manner. In some embodiments herein described, an IRES element is placed between two coding genes to allow for initiation of translation from an internal region of the mRNA. It allows the coordinated expression of two genes using the same promoter in a single gene cassette as will be understood by a person skilled in the art. Thus, the genes separated by IRES can be expressed from a bicistronic mRNA without requiring either cleavage of a polyprotein or generation of a monocistronic mRNA. In GV constructs, the use of IRES elements facilitates expression of downstream gvp genes—such as gvpC, gvpF, or gvpN—from a shared promoter in mammalian cells, ensuring balanced stoichiometry of GV structural and assembly proteins while preserving the integrity of each polypeptide product

Internal ribosome entry sites are approximately 450 nucleotides in length and are characterized by moderate conservation of primary sequence and strong conservation of secondary structure. The most significant primary sequence feature of the IRES is a pyrimidine-rich site whose start is located approximately 25 nucleotides upstream of the 3′ end of the IRES. Detailed information on IRES can be found in Jackson, et al., Trends Biochem. Sci., vol. 15, No. 12, pp. 477-483, 1990. Variants of IRES sequences and synthetic IRES mimetics can also be used, including those optimized for mammalian codon usage and RNA folding stability, as described in U.S. Pat. No. 11,761,008 B2.

Examples of IRES known in the art include IRES obtainable from picomavirus and IRES obtainable from viral or cellular mRNA sources such as for example, immunoglobulin heavy-chain binding protein (BiP), the vascular endothelial growth factor (VEGF) (Huezetal. (1998) Mol. Cell. Biol. 18 (11): 6178-6190), the fibroblast growth factor 2 (FGF-2), and insulin-like growth factor (IGFII), the translational initiation factor eIF4G and yeast transcription factors TFIID and HAP4, the encephelomycarditis virus (EMCV) which is commercially available from Novagen (Duke et al. (1992) J. Virol 66 (3): 1602-9) and the VEGFIRES (Huez et al. (1998) Mol Cell Biol 18 (11): 6178-90). IRES have also been reported in different viruses such as cardiovirus, rhinovirus, aphthovirus, HCV. Friend murine leukemia virus (FrMLV) and Moloney murine leukemia virus (MoMLV). As used herein, IRES encompasses functional variations of IRES sequences as long as the variation is able to promote direct internal ribosome entry to the initiation codon of a cistron.

In some embodiments, a separation element is a post-translation cleavage element comprising a cleavage site sequence. A post-translation cleavage element is typically placed between two adjacent coding genes. In the context of the present disclosure, IRES elements can thus be used to produce bicistronic or multicistronic GV expression systems (GVES) that yield correctly folded GV proteins within mammalian cells, contributing to the formation of distinct GV types based on transcriptional or translational stoichiometry.

In some embodiments, the post-translation cleavage element comprises a 2A element. The term “2A element” or “2A sequence” refers to a post-translational or co-translational processing cleavage site sequence. The 2A sequence can be a DNA sequence or the peptide expression produce of the DNA sequence. The latter is referred to as the 2A peptide. The 2A peptides are known to function by making the ribosome skip the synthesis of a peptide bond at the C-terminus of a 2A element, leading to separation between the end of the 2A sequence and the next peptide downstream. The cleavage occurs between the Glycine and Proline residues found on the C-terminus meaning the upstream cistron will have a few additional residues added to the end, while the downstream cistron will start with the Proline. The 2A elements used herein are placed between two adjacent GV coding genes. Exemplary 2A peptides are listed in Table 1 below:

TABLE 1
Exemplary 2A peptide sequences

| | | |
|---|---|---|
| P2A | ATNFSLLKQAGDVEENPGP | (SEQ ID NO: 15) |
| T2A | EGRGSLLTCGDVEENPGP | (SEQ ID NO: 16) |
| E2A | QCTNYALLKLAGDVESNPGP | (SEQ ID NO: 17) |
| F2A | VKQTLNFDLLKLAGDVESNPGP | (SEQ ID NO: 18) |
| BmCPV | DVFRSNYDLLKLCGDIESNPGP | (SEQ ID NO: 19) |
| BmIFV | TLTRAKIEDELIRAGIESNPGP | (SEQ ID NO: 20) |

In Table 1, the bold residues are the consensus residues among each type of 2A element (P2A, T2A, E2A or F2A). In each 2A element of Table 1, the cleavage occurs between the last G/P. In some embodiments, a linker sequence such as GAPGSG linker (SEQ ID NO: 21) is placed between a GV coding gene and the 2A sequence optionally using a linker, wherein any linker sequences such as GSG, GSGSG (SEQ ID NO: 2), SGS, and other linkers identifiable by a skilled person can be used. For example, a polynucleotide construct can comprise from 5′ to 3′ GV gene 1-GAPGSG-2A sequence-GV gene 2.

In some embodiments, the post-translation cleavage element comprises a cleavage recognition site that can be targeted and subsequently cleaved by protease enzymes. Exemplary protease enzymes include TEV, HCV NS3/5 protease, HIV protease, CMV protease, and HSV protease.

The term “protease cleavage site” in the sense of the disclosure indicates target sites for proteolytic cleavage by enzymes such as peptidases, proteases or proteolytic cleavage enzymes which break peptide bond between amino acids in proteins. The general nomenclature of cleavage site positions of the substrate were formulated by Schechter and Berger, 1967 [26] and Schechter

and Berger, 1968 Accordingly, the cleavage site is designated between P1-P1′, incrementing the numbering in the N-terminal direction of the cleaved peptide bond (P2, P3, P4, etc.). On the carboxyl side of the cleavage site the numbering is incremented in the same way (P1′, P2′, P3′ etc.).

Protease cleavage sites that can be inserted in engineered microcompartment proteins of the disclosure comprise regions up to 25 residues. In particular, protease cleavage sites are inserted in a configuration which makes them surface accessible. In some embodiments protease cleavage site are included in an unstructured segment or within an alpha helical or beta sheet secondary structured segment. Exemplary protease cleavage sites that can be inserted in engineered microcompartment proteins herein described comprise TEV protease cleavage sites with sequence ENLYFQG, (SEQ ID NO: 25) which is unstructured and others identifiable by a skilled person upon reading of the present disclosure (see Table 2).

Recognition sequences and cleavage sites of exemplary proteases are shown in Table 2./forward slash (/) indicates where protease cleaves the protein sequence.

TABLE 2
Recognition sequences and cleavage sites of exemplary proteases

| Enzyme Name | Sequence and Cleavage | SEQ ID NO |
|---|---|---|
| Human Rhinovirus (HRV) 3C Protease | LEVLFQ/GP | 22 |
| Enterokinase | DDDDK/ | 23 |
| Factor Xa | IEGR/ | 24 |
| Tobacco etch virus protease | ENLYFQ/G | 25 |
| (TEV protease) | | |
| Thrombin | LVPR/GS | 26 |
| NS3/4A | DLEVVT/STWV | 27 |
| NS4A/4B | DEMEEC/ASHL | 28 |
| NS4B/5A | DCSTPC/SGSW | 29 |
| NS5A/5B | EDVVCC/SMSY | 30 |
| NS4A/4B | DEMEEC/SQH | 31 |

In some embodiments, the cleavage recognition site comprises a TEV protease cleavable sequence that can be placed between two GV coding genes when the TEV enzymes are co-expressed. The TEV peptide can be cleaved to release the two GV proteins.

In some embodiments, the cleavage recognition site comprises a recognition sequence targeted by one or more non-structural protein NS3, NS4A, NS4B and NS5 sequence.

In some embodiments herein described, post-translation cleavage element comprises an intein or hedgehog family auto-processing domains or variants therefore, inserted in an open reading frame between multiple coding genes. The term “intein” refers to the protein equivalent of gene introns which facilitate protein splicing. The intein element contains the necessary components needed to catalyze protein slicing and often contains an endonuclease domain that participates in intein mobility (Perler, F. B., et al., Nucleic Acids Research 1994, 22, 1127).

The Hedgehog family auto-processing domains used herein comprise the hedgehog protein carboxy-terminal autocatalytic domain HhC. As a person skilled in the art will understand, the hedgehog (“Hh”) proteins are composed of two domains, an amino-terminal domain HhN, which has the biological signal activity, and a carboxy-terminal autocatalytic domain HhC, a carboxy-terminal autocatalytic domain HhC which cleaves Hh into two parts in an intramolecular reaction and adds a cholesterol moiety to the HhN. HhC has sequence similarity to the self-splicing inteins, the shared region is termed Hint. New classes of proteins containing the Hint domain have been discovered in bacteria and eukaryotes.

GVES and related constructs have been herein provided based on the surprising finding that an increased yield in GV type is obtained when a GVGC is expressed in mammalian cells using a construct including differentially expressed gene modules that are stoichiometrically configured to provide a relative excess of the primary structural GV protein (e.g., gvpA/B) as compared to the assembly factor (AF) proteins. This “macrooptimization” strategy, which provides a DI ratio of the structural protein to the assembly factors of at least 2:1 (e.g., 2:1:1 or 4:1:1), allows for enhanced expression in the mammalian cell of GV proteins encoded by the GVGC, resulting in the formation of a corresponding GV type with a significantly increased yield.

In particular stoichiometric Gas Vesicle Expression Systems (GVES), polynucleotide constructs, and related methods based on the surprising finding that the yield of functional gas vesicles in a mammalian cell can be dramatically increased by controlling the relative expression levels of the constituent GV proteins. This stoichiometric control, as described herein, provides a significant, non-obvious improvement over systems where gene cassettes are expressed at or near a 1:1 ratio.

This principle is referred to herein as “macrooptimization,” which involves configuring the system to provide a relative excess of the primary structural protein (e.g., GvpA or GvpB) as compared to the one or more assembly factor (AF) proteins (e.g., GvpN, J, K, F, G, W, V). This strategy departs from previous assumptions that one or more assembly factors might be the primary expression bottleneck. The embodiments provided herein demonstrate that providing the structural component at a significantly higher ratio is a key driver of high-yield GV formation, resulting in a system with greater sensitivity and improved signal output.

In a particular in some embodiments the GVES can be provided as a three differentially expressed gene moldules system. A first gene module (the “GvpA/B module”) encodes the structural protein gvpA. A second gene module (the “AF1 module”) encodes a first subset of assembly factors, for example, gvpN, gvpJ, gvpk, and gvpF. A third gene module (the “AF2 module”) encodes a second subset of assembly factors, for example, gvpG, gvpw, and gvpV. Each of these gene modules can be comprised in one or more cassettes and can be provided on a same or preferably separate vector, such as a separate lentiviral vector, allowing for co-transduction of a host cell at a desired ratio of viral particles, thereby achieving a target DI ratio.

In embodiments herein described, to quantify and control this stoichiometric relationship in stoichiometric GVES of the disclosure a “Dosage Index” (DI) is used.

As used herein, the term “Dosage Index” or “DI” refers to a quantitative parameter representing the relative stoichiometry of gene expression within a defined gene system, operon, or expression cassette set. The Dosage Index reflects the ratio between the genomic or vector-encoded dosage of individual genes or gene cassettes and the corresponding levels of their expressed products, such as messenger RNA or protein, thereby providing a normalized measure of expression balance among the components of the system. The DI is thus indicative of the proportional relationship between constituent genes of a multi-gene construct, for example a gas vesicle protein (gvp) cluster, and their translation products. An optimal or reference DI corresponds to the stoichiometric equilibrium necessary for correct assembly or function of the encoded structure or complex.

In one embodiment, the Dosage Index for a given gene i can be expressed as a ratio of the relative abundance of the expressed product (E_i) of gene i to the corresponding relative gene dosage (G_i), summed over n genes of the system. A DI value of approximately 1.0 indicates proportional, stoichiometrically balanced expression relative to the encoded gene dosage, whereas values significantly greater or less than 1.0 indicate overexpression or underexpression, respectively. The DI thereby provides a normalized, system-level descriptor of stoichiometric balance applicable to both single- and multi-vector systems. Variations in DI correlate with alterations in the efficiency of complex formation, assembly, or functional output, and the DI may be employed in conjunction with other system parameters, such as the Transduction-Expression Index (TEI), to evaluate or optimize system performance under defined Standard Conditions (SC).

Accordingly, as used herein, the wording “Dosage Index” refers to a normalized quantitative measure of the expression output of a gene cassette relative to a designated reference cassette, measured under standard conditions. This DI is determined by the regulatory architecture (e.g., promoter strength, UTRs, enhancers, mRNA stability elements) of the cassette, not by its physical copy number. For example, a GVES configured with a DI ratio of 4:1:1 expresses the first component at a 4-fold higher level than the second and third components, even when all three cassettes are present at a 1:1 copy number.

In embodiments, herein described the Dosage Index for a given gene i within a system comprising n genes is determined according to the following relationship:

$$DI\_i = E\_i / G\_i \qquad ( 1 )$$

where E_i represents the relative abundance of the expressed product of gene i, and G_i represents the relative dosage of the same gene, expressed as copy number, promoter strength, or molar fraction within the construct. The DI for a system can be reported as a vector of individual DI_i values or as a composite normalized average reflecting the overall stoichiometric balance of the system. When all constituent genes exhibit DI values approaching 1.0 under Standard Conditions, the system is deemed stoichiometrically balanced. Deviations from this equilibrium indicate disproportionate expression, which may adversely affect assembly or functionality of the encoded structure.

In embodiments herein described a gene expression system comprising a plurality of gene cassettes encoding functionally interrelated proteins can be characterized by the relative expression levels of the gene cassettes as defined by a Dosage Index. In such a system, the Dosage Index represents the ratio of expression output to gene dosage for each gene within the system, thereby reflecting the stoichiometric balance among the expressed components. An optimal functional output is achieved when the Dosage Index values of the constituent genes fall within the range of about 0.8 to 1.2, corresponding to substantially stoichiometric expression under Standard Conditions. Deviation of the Dosage Index beyond this range indicates overexpression or underexpression of one or more components, resulting in altered assembly, performance, or stability of the encoded functional complex

According to the present disclosure in a stoichiometric gas versicle expression system the DI of a gvpA/B differentially expressed gene module (gvpA/B-DI) is at least 2-fold higher than the DI of the two additional Assembly Factor differential module 1 (AF1-DI) and Assembly factors differential module 2 (AF2-DI). In particular the gvpA/B-DI:AF1-DI:AF2-DI extended ratio ranges from 2:1:1 to 6:1:1 possibly encompassing 5:1:1, 4:1:1 and 3:1; 1 extended ratios.

In certain preferred embodiments, the GVES comprises a three differentially expressed gene modules, for example in the form of a three-cassette system. A first cassette encodes the structural protein (e.g., gvpA), a second cassette encodes a first set of assembly factors (AF1, e.g., gvpNJKF), and a third cassette encodes a second set of assembly factors (AF2, e.g., gvpGWV). It was discovered that DI ratios of gvpA:AF1:AF2 of 2:1:1 and 4:1:1 provide a dramatic and unexpected increase in functional GV yield compared to a 1:1:1 ratio.

For example, embodiments configured with a 4:1:1 DI ratio produced a BURST Signal-to-Background Ratio (SBR) of approximately 118, while a 2:1:1 ratio produced an SBR of approximately 85. Both were substantially and surprisingly superior to the SBR of approximately 10 produced by a 1:1:1 ratio. Therefore, in preferred embodiments, the DI ratio of the structural protein cassette to each of the assembly factor cassettes is at least 2:1, such as 2:1:1, 3:1:1, 4:1:1, 5:1:1 or a range therebetween, such as from 2:1:1 to 6:1:1.

This macrooptimization principle is also demonstrated in other configurations. For example, in a two-vector or two-cassette system, a first cassette encodes the structural protein (gvpA) and a second cassette encodes the full set of assembly factors (AF). In such embodiments, DI ratios of gvpA:AF of 2:1 and 4:1 were both shown to produce a significantly higher SBR (e.g., an SBR of approximately 6-7) compared to a 1:1 ratio (e.g., an SBR of approximately 1.5).

Accordingly, the systems and methods described herein, in several embodiments, utilize a GVES characterized by a stoichiometric DI ratio wherein the primary structural protein is expressed at a level at least 2-fold higher, and preferably 4-fold higher, than one or more assembly factor cassettes. This configuration achieves a high-yield system capable of producing a BURST SBR of greater than 50, greater than 75, or greater than 100 in vitro under standard assay conditions, thereby achieving a high-yield system suitable for sensitive detection in vitro and in vivo.

In some embodiments, Stoichiometric GVES herein described can include gene cassettes in a further refined stoichiometry, herein referred to as “microoptimization,” has been identified. This “microoptimization” involves the stoichiometric balancing within the set of assembly factor (AF) cassettes. It was discovered that a lower relative expression of the assembly factor subgroup containing gvpJ and gvpK (referred to herein as the AF1 cassette, e.g., gvpNJKF) as compared to the other assembly factor subgroup (referred to herein as the AF2 cassette, e.g., gvpGWV) is also beneficial and contributes to the high-yield phenotype.

Thus, in certain preferred embodiments, the GVES is configured such that the DI ratio of the AF1 cassette to the AF2 cassette is less than 1:1 . . . . This configuration, wherein the expression of the AF1 assembly factors is lower relative to the AF2 assembly factors, contributes to the overall enhanced yield of the GV type formation in the mammalian cell. This microoptimization can be achieved by, for example, utilizing a weaker promoter for the AF1 cassette, engineering a dual-promoter configuration on the AF1 cassette that results in partial silencing, or utilizing other regulatory elements that reduce the final expression output of the AF1 cassette relative to the AF2 cassette.

In preferred embodiments, the gvpA/B gene module, AF1 gene module, and the AF2 gene module are configured such that the effective DI ratio of AF1-DI to AF2-DI is less than 1:1. This configuration, wherein the expression of the AF1 assembly factors is lower relative to the AF2 assembly factors, contributes to the overall enhanced yield of the GV type formation in the mammalian cell.

In certain embodiments, this configuration wherein the effective Dosage Index (DI) ratio of the AF1 module (AF1-DI) to the AF2 module (AF2-DI) is less than 1:1 is achieved through specific quantitative relationships. For example, the AF1-DI may be configured to be substantially less than the AF2-DI, such as between approximately 10% and 95% of the AF2-DI. In more specific embodiments, the AF1-DI may be between approximately 25% and 75% of the AF2-DI, or between approximately 40% and 60% of the AF2-DI. This controlled reduction in the relative expression of the AF1 module contributes to optimizing the assembly process and overall yield of functional gas vesicles.

In other embodiments, the microoptimization principle wherein AF1-DI is less than AF2-DI can be achieved based on the relative strengths of the regulatory elements employed. In one such embodiment, the AF1 gene module is placed under the control of a mammalian promoter and/or associated regulatory elements that are demonstrably weaker than the promoter and/or regulatory elements controlling the AF2 gene module, when measured under identical standard conditions. This differential regulatory strength inherently establishes the desired expression imbalance where AF1-DI is less than AF2-DI.

In another variation, the lower effective AF1-DI relative to AF2-DI is achieved not solely through promoter strength, but through the differential use of other regulatory elements affecting mRNA stability, translational efficiency, or protein stability. For instance, the AF1 module may lack certain post-transcriptional stabilizing elements (e.g., WPRE) that are present in the AF2 module, or it may incorporate specific sequences in its UTRs or coding region that lead to reduced mRNA half-life or translational initiation compared to the AF2 module. Alternatively, the proteins encoded by the AF1 module could be engineered to include degradation tags absent from the AF2 proteins, resulting in a lower steady-state protein level and thus a lower effective AF1-DI. These different approaches (promoter strength, stability elements, degradation tags) can be used individually or in combination to achieve the desired ratio.

In the most preferred embodiments, the GVES is configured to utilize both macrooptimization and microoptimization principles. Such systems are characterized by an overall DI ratio of gvpA:AF1:AF2 wherein the gvpA DI is at least 2-fold higher than the AF DIs, and the AF1 DI is lower than the AF2 DI. Examples of such overall DI ratios comprise, 4:0.8:1, 4:0.5:1, 2:0.5:1, 4:1:1.2, or 2:0.8:1. This combined optimization results in a highly robust and efficient system for producing functional gas vesicles in mammalian cells.

In some embodiments, the stoichiometric ratios can be obtained through approaches comprising selection of regulatory sequences and/or post-translational regulatory elements for each differentially expressed gene module that will achieve the DI ratios in the sense of the disclosure, as will be understood by a skilled person upon reading of the present disclosure.

In those embodiments, computational modeling and synthetic biology design tools are used to predict regulatory strength and dynamic range, optimizing expression for vesicle assembly and acoustic performance.

In some embodiments, computational modeling and synthetic biology design tools are used to predict regulatory strength and dynamic range, optimizing expression for vesicle assembly and acoustic performance. Such computational approaches can include in-silico simulation of transcriptional and translational processes, thermodynamic modeling of promoter-transcription-factor interactions, codon-usage optimization, mRNA secondary-structure prediction, and kinetic modeling of protein expression and folding. In particular, gene regulatory networks can be modeled using ordinary-differential-equation (ODE) or stochastic frameworks to determine promoter strength, ribosome-binding-site efficiency, and degradation constants suitable for balanced co-expression of gvpA/B, gvpC, and accessory gvp genes.

In some embodiments, synthetic-biology design platforms—such as SBOL-compliant DNA-assembly software, BioStudio, Benchling, Geneious, or MATLAB-SimBiology—can be used to construct and simulate GV expression cassettes prior to experimental implementation. Computational pipelines may integrate experimentally derived data (for example, RNA-seq or proteomics) with machine-learning models trained to predict transcriptional strength, translation rate, or post-translational modification patterns in a given host cell. These models enable identification of regulatory architectures that yield optimal stoichiometry of GV proteins and stable vesicle formation.

In certain embodiments, multiscale modeling is employed to connect molecular-level expression parameters to macroscopic acoustic or magnetic properties of the resulting gas vesicles. Finite-element simulations and molecular-dynamics calculations can be used to estimate GV shell stiffness, resonance frequency, and collapse threshold as a function of GvpC length or modification state. Design algorithms can iteratively refine promoter choice, separation elements (e.g., IRES, 2A), and codon usage to achieve a target acoustic signature. Accordingly, computational and synthetic-biology optimization methods described herein provide predictive control over GV gene expression and physical performance, reducing empirical screening requirements and enabling rational design of GV types for specific imaging or therapeutic applications.

In some embodiments, the expression of the differentially expressed gene module can also be regulated by post-translational modifications. As a person skilled in the art will understand, the sequences of the inserted auto-processing polypeptides or cleavage sites can be manipulated to enhance the efficiency of expression of the separate proteins.

In some embodiments the genetic constructs encoding the GVES can be provided in various architectures, such as being comprised in one, two, three, or more separate polynucleotides, vectors, or cassettes. In a particular the GVES can be provided as a three-cassette or three-vector system. A first cassette (the “GvpA/B cassette”) encodes the structural protein gvpA. A second cassette (the “AF1 cassette”) encodes a first subset of assembly factors, for example, gvpN, gvpJ, gvpk, and gvpF. A third cassette (the “AF2 cassette”) encodes a second subset of assembly factors, for example, gvpG, gvpw, and gvpv. Each of these cassettes can be provided on a separate vector, such as a separate lentiviral vector, allowing for co-transduction of a host cell at a desired ratio of viral particles, thereby achieving the target DI ratio. For example, the genes from the Anabaena flos-aquae (Ana) GV gene cluster, comprising gvpA, gvpN, gvpJ, gvpK, gvpF, gvpG, gvpW, and gvpV, can be distributed across multiple constructs to improve delivery efficiency and enable stoichiometric tuning.

In a particularly preferred embodiment, the GVES is provided as a system comprising three distinct gene expression cassettes, preferably on three separate vectors. A first gene expression cassette (the “GvpA/B cassette”), which is monocistronic, comprises a coding region for the structural protein gvpA. A second gene expression cassette (the “AF1 cassette”), which is polycistronic, comprises a coding region for a first subset of assembly factors, for example, gvpN, gvpJ, gvpk, and gvpF. A third gene expression cassette (the “AF2 cassette”), which is also polycistronic, comprises a coding region for a second subset of assembly factors, for example, gvpG, gvpW, and gvpV. Each of these cassettes can be provided on a separate vector, such as a separate lentiviral vector, allowing for co-transduction of a host cell at a desired ratio of viral particles, thereby achieving the target DI ratio.

In another embodiment, the system is provided as a system comprising two distinct gene expression cassettes, preferably on two separate vectors. For example, a first gene expression cassette, which is monocistronic, encodes the structural protein (gvpA), while a second “AF cassette,” which is polycistronic, comprises a coding region for all seven assembly factors (gvpNJKFGWV) divided in two AF1 and AF2 differentially expressed gene modules operatively linked to regulatory sequences to allow a differential expression with the DI ratios accordance with the present disclosure. For example the genes within this polycistronic cassette can be operatively linked by, for example, 2A self-cleaving peptides or Internal Ribosome Entry Sites (IRES). This architecture allows for the stoichiometric macrooptimization of the gvpA cassette expression relative to the complete set of assembly factors. In such embodiments, the desired Dosage Index (DI) ratio (e.g., 2:1 or 4:1) is achieved by delivering the two vectors at the corresponding particle ratio.

In another embodiment, all components of the GVES are provided on a single polynucleotide or vector, wherein said single vector comprises three distinct gene expression cassettes. In this configuration, the single vector comprises: a first monocistronic expression cassette (e.g., [Promoter 1]-[gvpA]-[Terminator 1]); a second polycistronic expression cassette (e.g., [Promoter 2]-[AF1 genes, e.g., gvpNJKF]-[Terminator 2]); and a third polycistronic expression cassette (e.g., [Promoter 3]-[AF2 genes, e.g., gvpGWV]-[Terminator 3]). The stoichiometric macrooptimization and/or microoptimization is achieved in this embodiment by selecting regulatory elements (e.g., Promoter 1, Promoter 2, Promoter 3) that inherently provide the desired relative Dosage Index ratios (e.g., a 4:1:1 ratio) upon expression, even though the physical copy number of the cassettes on the single vector is 1:1:1.
a second single-vector configuration, all components of the GVES are provided within a single, complex polycistronic cassette under the control of a single promoter. For example, the construct may be arranged as [Promoter 1]-[gvpA]-[IRES 1]-[AF1 genes]-[2A peptide]-[AF2 genes]-[Terminator 1]. In this embodiment, the desired stoichiometric DI ratios are achieved not by separate promoters, but by the differential translational efficiency of the regulatory linkers (e.g., IRES elements, 2A peptides) and other post-transcriptional elements that govern the final protein-level dosage of each component, thereby achieving the desired macrooptimization and/or microoptimization from a single transcript.

The term “polycistronic construct” as used herein refers to a construct capable of simultaneously translating multiple genes from a single transcript as will be understood by a person skilled in the art, within a single cassette or in different cassettes on the construct if the cassettes are separated by an internal ribosome entry site.

In some embodiments, the polycistronic construct can be a biocistronic construct which comprises two genes separated by an Internal Ribosome Entry Site (IRES) element which allows for initiation of translation from an internal region of the mRNA. Use of IRES allows for the upstream protein to remain pristine while the downstream protein gets a MATT peptide addition to its N terminus. The second protein may be expressed at a lower level compared with the first protein since the ribosome entry site is less efficient than the 5′cap/UTR as will be understood by a skilled persons.

In some embodiments, some of the gene of a GVGC are expressed at a lower level compared to other gvp genes of the GV gene cluster when expressed under a same promoter and regulatory regions in those embodiments, the stoichiometry of the expression of the specific cassette can be increased to provide an optimal functionality of the GVES in the mammalian cell.

In some embodiments the booster constructs can be comprised on one or more gene cassettes, where the stability of the transcript can tune stoichiometry of the translated proteins. Regulatory elements that stabilize mRNAs (for example PolyA, WPRE) can be used on the booster constructs. For genes that need to be expressed at lower relative stoichiometries, these stability elements can be removed, or can be conditionally removed using siRNAs/shRNAs/aptazymes/cas9 and etc. While the other GV cassette can include these mRNA stability elements.

In some embodiments the booster constructs can be comprised on one or more gene cassettes, where the use of degradation tags can tune stoichiometry of the translated proteins. Degradation tags target proteins for proteolysis, for example ubiquitin and library of ubiquitin-fusion degradation tags (UbR, UbP, UbW, UbH, UbI, UbK, UbQ, UbV, UbL, UbD, UbN, UbG, UbY, UbT, UbS, UbF, UbA, UbC, UbE, UbM, 3×UbVR, 3×UbVV, 2×UbVR, 2×UbVV, UbAR, UbVV, UbVR, UbAV, 2×UbAR, 2xUbAV), auxin-inducible degraon (AID), D-element, the PEST sequence, unstructured initiation sites, or short sequences rich in acceptor lysines. Genes on the booster constructs will not have these degradation tags while relatively degradation tags can be used for the other genes that need to be expressed to lower levels. This can be used in combination with promoters and transcript stability examples.

Some embodiments the booster constructs can be comprised on one or more gene cassettes, where the use of micro-ORFs upstream of a cassette (ORF encoding gv genes) can be used to reduce the expression of GV proteins. Micro-ORFs are short open reading frames placed up stream of the ORF encoding the protein(s) of interest and results in the suppression of protein expression. They include a kozak/start codon NNNATG, small peptide and stop codon (TGA, TAG, TAA), for example AAAATGGCCGCGCCCAGAGCGTAG (SEQ ID NO: 481), NNNATG (NNN) [TAG/TGA/TAA] (SEQ ID NO: 474) ([28]). For genes that need to be expressed at lower relative stoichiometries, mico-ORFs can be placed upstream of their cassette to reduce the expression level of these GV proteins.

In some embodiments the constructs can be comprised on one or more gene cassettes, where the use of different inducible promoters (chemically or otherwise) can tune stoichiometry of translated proteins. Different promoters that are inducible by different stimuli can be used to drive expression of the booster construct and/or other cassettes. A higher amount of inducer can be used to increase the expression of booster constructs. For genes that need to be expressed at lower relative stoichiometries a relatively lower amount of inducer can be used.

In some embodiments the constructs can be comprised on one or more gene cassettes, where the presence of enhancing introns can tune stoichiometry of the translated proteins. Intron-mediated enhancement can be used on the booster constructs. For genes that need to be expressed at lower relative stoichiometries, these introns can be omitted, while the other GV cassette can include these introns. ([29], [30])

In some embodiments the constructs can be comprised on one or more gene cassettes, where the stoichiometry of the translated proteins can be tuned by different modes of Ribosome entry. Translation of the booster construct can be initiated via the stronger cap-dependent gene expression mediated by the KOZAK sequence and genes that need to be expressed at lower relative stoichiometries can be initiated via Internal Ribosome Entry Site (IRES).

In some embodiments, the relative stoichiometry of proteins expressed from one or more differentially expressed gene modules is modulated by controlling the copy number of the module or of the genetic construct carrying it within the host cell. The term “copy number” as used herein refers to the number of instances of a gene, cassette, or vector present within a cell, genome, or episomal environment. Variation in copy number directly influences transcript abundance and, consequently, protein concentration. By adjusting the copy number of specific GV expression cassettes or vectors, the ratio of different Gvp proteins can be fine-tuned to yield the desired vesicle morphology, size, and collapse threshold.

Copy-number control can be achieved at several levels, including: (i) Vector backbone selection, wherein the origin of replication determines the plasmid copy number (for example, high-copy ColE1 origin versus low-copy p15A or single-copy BAC systems); (ii) Selectable marker pressure, where differential antibiotic concentrations or auxotrophic complementation maintain defined plasmid ratios; (iii) Targeted genomic integration, wherein constructs are inserted into loci of known transcriptional activity and copy number using recombinase- or CRISPR-mediated approaches; and (iv) Episomal replication elements such as EBV oriP/EBNA1 or S/MAR sequences that permit tunable maintenance of extrachromosomal copies in mammalian cells. Through these mechanisms, a single cell population can be engineered to contain higher copy numbers of one GV module (for example, the gvpA/B module) and lower copy numbers of another (for example, the gvpC module), thereby establishing the stoichiometric balance required for efficient GV assembly.

In some embodiments, the desired DI ratio between differentially expressed gen module can be achieved using multiple constructs or vectors each encoding one or more distinct GV module which are co-transfected or co-transduced into a host cell. The relative dosage of each construct determines the transcriptional output of its encoded genes. For instance, introducing a threefold molar excess of a gvpA/B expression construct relative to a gvpC construct can produce a vesicle population enriched in shell-forming subunits with reduced outer-surface reinforcement, resulting in GVs that collapse at lower acoustic pressures and exhibit increased nonlinear response. Conversely, increasing the relative copy number of gvpC or accessory genes can yield mechanically stronger vesicles with higher collapse thresholds.

Such modulation of inter-construct ratios enables programmable control of GV phenotype without altering the underlying promoter or regulatory architecture.

Copy-number-based stoichiometric tuning can also be dynamically regulated by inducible replication or amplification systems, including those based on Cre-lox-mediated excision or amplification, recombinase directionality factors, or drug-responsive replication origins. For example, a replication origin that activates in response to doxycycline or temperature change can be used to transiently increase expression of one GV module during an assembly phase and reduce it afterward, producing vesicles with defined temporal assembly kinetics.

Integration of copy-number control with differential regulatory design allows hierarchical expression programs, wherein transcriptional and translational tuning are reinforced by genomic dosage effects.

Accordingly, the stoichiometric output of GV expression systems described herein can be determined by the combined effect of (i) internal regulatory elements within a differentially expressed gene module and (ii) the relative copy number of those modules or constructs across the cellular population. Together, these parameters permit precise, quantitative modulation of gas vesicle protein composition, ensuring reproducible generation of GV types with defined acoustic, magnetic, or optical properties in mammalian, prokaryotic, or cell-free systems.

Accordingly in some embodiments, all components of the GVES can be encoded on a single polynucleotide, vector, or cassette or in two or more polynucleotide, vector, or cassette. For example, a single construct can comprise a monocistronic gene expression cassette encoding gvpA and a polycistronic gene expression cassette encoding all assembly factors, gvpNJKFGWV. The individual genes within the polycistronic AF cassette may be operatively linked by 2A peptides, and the gvpA cassette may be linked to the AF cassette by an IRES, all under the regulatory control of a single promoter.

In a particularly preferred embodiment, the GVES is provided as a system comprising three distinct gene expression cassettes, preferably on three separate polynucleotide constructs.

In those embodiments the gvpA/B gene module AF1 module and AF2 module are comprised in separate gene expression cassettes, and the gvpA/B gene expression cassette, the AF1 gvp gene expression cassette, and the AF2 gvp gene expression cassette are comprised on at least three distinct polynucleotides, such as three separate vectors. This multi-construct architecture is particularly advantageous when using delivery vehicles with a limited packaging capacity, such as viral vectors, including but not limited to lentiviral or adeno-associated viral (AAV) vectors.

For example, the complete 8-gene cluster from Anabaena flos-aquae (Ana), along with necessary promoters, terminators, and other mammalian regulatory elements, can easily exceed the approximately 8 to 10 kilobase (kb) packaging limit of a single lentiviral vector. Distributing these components across three separate, smaller constructs (e.g., a gvpA/B vector of ˜4 kb, an AF1 vector of ˜6.7 kb, and an AF2 vector of ˜4.8 kb) ensures that each individual construct is well within the acceptable packaging limit, leading to higher viral titers and more efficient delivery.

In this multi-construct embodiment, the stoichiometric control, characterized by the desired Dosage Index (DI) ratios, is achieved by controlling the relative copy number or multiplicity of infection (MOI) of each distinct polynucleotide delivered to the host cell. For example, to achieve the desired gvpA:AF1:AF2 DI ratio of 4:1:1, a population of host cells is co-transduced with a 4-fold excess of the gvpA/B vector relative to the AF vector and the AF2 vector.

This multi-construct, co-transduction approach has been demonstrated to be surprisingly effective, enabling efficient transduction of hard-to-transduce primary cells (such as primary human T cells) and resulting in a robust, high-yield system. This architecture provided the highest observed signal, for instance, a BURST SBR of approximately 118 (mean±8.4 s.e.m.) at a 4:1:1 ratio, demonstrating a substantial improvement over other configurations. In this embodiment, the gvpA/B cassette is preferably a monocistronic cassette, while the AF1 cassette (encoding, e.g., gvpNJKF) and the AF2 cassette (encoding, e.g., gvpGWV) are preferably polycistronic cassettes, each under the control of their own respective promoter.

In those embodiments the Dosage Index can be measured or correlated with the Transduction-Expression Index under the same Standard Conditions, such that variations in DI account for differential efficiency of delivery, transcription, translation, or assembly affecting TEI values.

As used herein, the term “Transduction-Expression Index” or “TEI” refers to a dimensionless, normalized measure of end-to-end gene-to-function efficiency under Standard Conditions. The TEI is defined as the functional ultrasound output from an acoustic reporter gene (ARG) system per effective input, normalized to a designated reference system scored as 1.0 under Standard Conditions. Functional output is quantified using suitable ultrasound readouts comprising collapse-based methods such as BURST (Burst Ultrasound Reconstruction with Signal Template ultrasound readouts or non-collapse based methods utilizing nonlinear buckling signals (e.g., xAM or Amplitude Modulation Pulse Sequence), expressed as signal-to-background ratio (SBR) or an equivalent validated metric obtained with the same imaging parameters. The TEI is thereby calculated as the mean BURST SBR per effective expressing cell for the system of interest divided by that of a designated reference system, measured under the same Standard Conditions.

The Transduction-Expression Index (TEI) is defined according to the relationship:

TEI=(Output/Input)_System/(Output/Input)_Reference

where Output corresponds to the measured functional ultrasound signal, typically expressed as the mean BURST signal-to-background ratio (SBR) across a designated region of interest and acquisition time window, and Input corresponds to the effective number of cells at the time of imaging contributing to the signal, which represents the number of cells successfully expressing functional gas vesicles, approximated experimentally by the number or fraction of transduced cells (e.g., determined by flow cytometry for reporter markers) within the imaged population, particularly when comparing systems where delivery efficiency itself might differ. The ratio of the normalized Output to Input, relative to that of the designated reference system, yields a dimensionless TEI value that quantitatively represents the overall efficiency with which an introduced gene or cassette set produces a functional acoustic output encompassing factors influencing both delivery and post-delivery expression and assembly efficacy. The TEI thereby integrates the contributions of delivery efficiency, transcriptional and translational yield, macromolecular assembly, and acoustic performance into a single normalized measure of system efficacy.

The DI and TEI together provide a dual-parameter framework linking molecular stoichiometry to macroscopic function. The DI captures the relative balance of expression among gene cassettes, reflecting how dosage and expression ratios contribute to the proper assembly of multi-component complexes, whereas the TEI captures system-level performance encompassing delivery, transcription, translation, assembly, and functional output. Variations in DI can therefore explain variance in TEI, as imbalanced stoichiometry among gene products may impair proper assembly and reduce functional output. Used jointly, the two indices enable quantitative analysis and optimization of gene expression systems for desired functional performance under standardized experimental or operational conditions.

For purposes of the present disclosure, the Dosage Index (DI) and the Transduction-Expression Index (TEI) are each determined or interpreted with respect to Standard Conditions (SC), which establish a reproducible experimental and analytical framework for comparing gene expression systems. As used herein, Standard Conditions encompass a defined set of physical, biological, and instrumental parameters, including but not limited to the identity and configuration of the imaging apparatus, transducer frequency and pressure, pulse sequence, acquisition frame count, environmental temperature, imaging depth, and data analysis pipeline. The use of Standard Conditions ensures that variations observed in DI or TEI arise from differences in biological or genetic parameters rather than from experimental artifacts or inconsistent measurement settings. Under SC, the reference system—typically a single-construct acoustic reporter gene (ARG) expressing a defined gas vesicle (GV) cluster—is assigned a TEI value of 1.0, providing a baseline for normalization and comparative evaluation.

Under Standard Conditions, variations in DI modulate the expression and assembly components that contribute to TEI. For example, an increase in the DI of a structural gene relative to a scaffold or chaperone gene may disrupt stoichiometric balance, resulting in aberrant complex formation and a corresponding reduction in TEI. Conversely, maintenance of DI values within an empirically determined optimal range, typically between about 0.8 and 1.2, supports balanced expression and efficient assembly, yielding TEI values comparable to or exceeding the reference standard. Accordingly, the combined use of DI and TEI under Standard Conditions provides a quantitative and mechanistic framework for optimizing gene expression systems, enabling predictive adjustment of gene dosage, promoter strength, or cassette ratios to achieve desired functional outcomes.

In embodiments herein described, the polynucleotide constructs of the GVES, GVRMC, or GVRGC, including the gvpA/B module, the AF1 module, and the AF2 module, whether as separate constructs or combined onto one or more constructs, are provided as part of a vector or “delivery vehicle.” The term “vector” indicates a molecule configured to be used as a vehicle to artificially carry foreign genetic material into a cell, where it can be replicated and/or expressed. An expression vector is a vector configured to carry and express the material in a cell under appropriate conditions.

In some embodiments, a suitable vector can comprise a recombinant plasmid, a recombinant non-viral vector, or a recombinant viral vector. These vectors comprise the polynucleotide constructs and the appropriate regulatory elements such as promoters, enhancers, and terminators compatible with the mammalian cell intended to heterologously express the GV type.

In preferred embodiments, particularly for delivery into primary cells, immune cells, and for in vivo applications, the vectors are viral vectors. Exemplary viral vectors include, but are not limited to, lentiviral vectors, adeno-associated viral (AAV) vectors, adenoviral vectors, retroviral vectors (including gammaretroviral vectors), baculoviral vectors, and herpes simplex virus (HSV) vectors. As demonstrated in the Examples, lentiviral vectors are particularly suitable for delivering the multi-construct system to hard-to-transduce cells, such as primary human T cells, and achieving robust expression and are thus particularly preferred.

In other embodiments, the polynucleotide constructs can be delivered as non-viral vectors. Examples of non-viral delivery methods include, but are not limited to, plasmid DNA, “naked” DNA or RNA (such as mRNA), lipid nanoparticles (LNPs), liposomes, polyplexes (e.g., using polyethyleneimine or PEI), dendrimers, electroporation, sonoporation, magnetofection, or particle-based delivery (e.g., gene gun). The polynucleotide construct can also be part of a transposon system, such as piggyBac or Sleeping Beauty, which facilitates genomic integration upon delivery.

Accordingly, in certain preferred embodiments, the genetically engineered Gas Vesicle Expression System (GVES) is provided as a combination of two or more distinct polynucleotide constructs, such as a set of separate vectors, wherein the differentially expressed gene modules are partitioned across said constructs. This multi-construct or multi-vector architecture is particularly advantageous for delivering large, multi-gene clusters, such as the 8-gene cluster from Anabaena flos-aquae, using delivery vehicles that have a limited packaging capacity. For example, the total size of a single construct encoding all GVGC modules and necessary mammalian regulatory elements may exceed the optimal packaging limit of viral vectors, such as lentiviral vectors (approximately 8-10 kb), which can lead to reduced viral titers and inefficient gene delivery.

In a particularly preferred embodiment, the GVES is configured as a set of three distinct polynucleotide constructs or vectors. In this configuration, a first polynucleotide construct comprises the gvpA/B differentially expressed gene module, a second polynucleotide construct comprises the AF1 gvp differentially expressed gene module (e.g., encoding GvpN, J, K, and F), and a third polynucleotide construct comprises the AF2 gvp differentially expressed gene module (e.g., encoding GvpG, W, and V). This partitioning allows each individual construct to be well within the packaging limits of, for example, lentiviral vectors, thereby facilitating high-titer viral production and more efficient co-delivery. This multi-construct approach enables the stoichiometric control required for macrooptimization to be achieved at the level of delivery by controlling the relative copy number or dosage of each vector introduced into the host cell.

To practice this embodiment, a method for expressing Gas Vesicles involves co-transducing the target mammalian cell population with a composition comprising the set of vectors at a predetermined relative ratio. For example, to achieve the desired Dosage Index (DI) ratio wherein the gvpA/B-DI is at least 2-fold higher than the AF1-DI and AF2-DI, the cells are transduced with a relative ratio of transducing units (e.g., Multiplicity of Infection or MOI) of the first (gvpA/B) vector to the second (AF1) and third (AF2) vectors in a range from 2:1:1 to 4:1:1. Delivering the vectors at a ratio of approximately 4:1:1, for instance, has been shown to be highly effective, resulting in robust expression and a superior functional ultrasound signal compared to other ratios or single-vector systems. This multi-vector delivery strategy has proven surprisingly effective for engineering hard-to-transduce cells, such as primary T cells, and achieves a significantly higher Transduction-Expression Index (TEI) than a comparable single-construct system under Standard Conditions. The polynucleotide constructs in this set are preferably viral vectors, such as lentiviral vectors, but can also include adeno-associated viral (AAV) vectors, or non-viral vectors.

In embodiments herein described, the GVES comprising a GVGC in two or more gene cassettes located on one or more polynucleotide construct herein described operatively connected to regulatory sequences can be introduced to a mammalian host allowing expression of the GV constructs and producing of gas vesicles in the mammalian host.

A significant advantage of this multi-construct system is its ability to provide good transduction efficiency and surprisingly robust gene expression in mammalian cells that are typically considered difficult to engineer. This includes, in particular, clinically relevant primary cells and immune cells, such as primary human T cells. Prior to the present invention, achieving stable, high-yield expression of the complete polycistronic GV gene cluster in these cell types was a significant technical barrier. The combination of overcoming viral packaging limits, which provides for more efficient delivery, and the stoichiometric optimization, which provides for a high functional yield per cell, enables for the first time the robust and detectable expression of acoustic reporters in these therapeutically important cell populations.

As used herein, the term “immune cell” refers to any cell of hematopoietic origin that participates in innate or adaptive immune responses and that can be genetically engineered to express gas vesicle constructs (GVES, GVPC, or GVRGC) as described herein. Immune cells include, without limitation, lymphoid lineage cells such as T lymphocytes (including CD4 helper T cells, CD8 cytotoxic T cells, γδ T cells, and regulatory T cells), B lymphocytes, and natural killer (NK) cells; myeloid lineage cells such as monocytes, macrophages, dendritic cells, neutrophils, eosinophils, basophils, and mast cells; and precursor or progenitor forms thereof derived from bone marrow or hematopoietic stem cells. Immune cells can be obtained directly from a mammalian host (for example, human peripheral blood mononuclear cells, spleen, thymus, lymph node, or bone marrow) or generated ex vivo through expansion and differentiation of stem or progenitor cells.

Immune cells can be engineered using viral or non-viral vectors to express GV constructs for use as contrast-generating reporter cells, biosensors, or therapeutic carriers. In particular, immune cells expressing GVs can be used to report localization, migration, activation, or cytotoxic events in vivo under ultrasound or MRI, as described in U.S. Pat. Nos. 11,761,008 B2 and 11,446,523 B2. The intracellular environment of immune cells provides post-translational machinery (such as proteases, kinases, and redox regulators) that can influence the structure or modification state of the GvpC protein, thereby generating immune-cell-specific GV types with unique acoustic or magnetic characteristics. Accordingly, “immune cell” in the sense of the disclosure encompasses both unmodified and engineered immune cells of innate or adaptive lineages, including autologous, allogeneic, or xenogeneic sources, suitable for research, diagnostic, or therapeutic use.

As used herein, the term “primary cell” refers to a cell that has been directly isolated from a mammalian tissue or organ and retains the differentiated characteristics, genetic profile, and physiological responses of its in vivo origin. Primary cells are distinct from immortalized or transformed cell lines in that they have limited replicative potential and more faithfully recapitulate the biochemical and structural context of native tissue. Primary cells can be harvested from a wide range of mammalian tissues, including but not limited to blood, liver, lung, heart, brain, kidney, pancreas, skin, muscle, and connective tissues, using mechanical, enzymatic, or perfusion-based dissociation methods followed by culture under defined growth conditions.

Exemplary primary cell types include hepatocytes, cardiomyocytes, neurons, astrocytes, microglia, fibroblasts, endothelial cells, keratinocytes, adipocytes, pancreatic B-cells, renal epithelial cells, and smooth or skeletal muscle cells. Primary immune cells such as macrophages, dendritic cells, monocytes, T cells, B cells, and NK cells are also encompassed. Primary cells can be genetically engineered in vitro or ex vivo using transfection, transduction, electroporation, or mRNA delivery methods to express GV gene clusters or related constructs (GVES, GVPC, or GVRGC) to form gas vesicle structures or reporters within their cytoplasm.

As described in U.S. Pat. No. 11,761,008 B2, mammalian primary cells engineered with GV constructs can assemble stable gas vesicles that retain detectability under MRI or ultrasound while preserving cellular physiology. In certain embodiments, GV expression in primary cells can be regulated by inducible or tissue-specific promoters to ensure controlled expression and compatibility with therapeutic applications. Accordingly, “primary cell” in the sense of the present disclosure encompasses any non-immortalized mammalian cell that is directly isolated from a tissue and capable of genetic modification to express, assemble, or host gas vesicle nanostructures in vitro, ex vivo, or following reintroduction into a mammalian subject.

In particular in some embodiments, the method comprises introducing into the mammalian cell a genetically engineered Gas Vesicle expression system (GVES) herein described for a time and under condition to allow expression of the gvp genes in the mammalian cell.

In some embodiments, the method comprises introducing into a cell of the mammalian host a genetically engineered Gas Vesicle expression system (GVES) herein described in which the gvp genes encode for proteins of the gas vesicle type, the introducing performed for a time and under condition to allow expression of the gvp genes in the mammalian cell.

Expression of GV constructs in a mammalian cell can be performed by cloning one or more polynucleotides encoding naturally occurring GV proteins or homologs thereof that are required for production of GVs (comprising gvpB, gvpN, gvpF, gvpG, gvpL, gvpS, gvpK, gvpJ, and gvpU and other proteins known to those skilled in the art and described herein) into one or more suitable constructs configured to express the heterologous GV proteins in the mammalian cell. Polynucleotides encoding GV protein genes can be cloned using commercially available reagents from vendors such as Qiagen, Invitrogen, Applied Biosystems, Promega, New England BioLabs and others, following standard molecular biology methods known in the art, such as those described herein. As would be understood by those skilled in the art, polynucleotides encoding GV protein genes can be obtained from several different sources. For example, polynucleotides encoding GV proteins can be obtained by isolating genomic DNA or cDNA encoding GV proteins from microorganisms whose genomes encode GV proteins genes, and/or express GV proteins RNA. RNA can be isolated from a cell that expresses GV proteins genes, and cDNA produced by reverse transcription using standard techniques and commercial kits. Genomic DNA can be purified from the cell, and cDNA or genomic DNA encoding one or more GV proteins isolated, following methods known to those in the art. In addition or in the alternative, polynucleotides comprising one or more gas vesicle genes can be synthesized using oligonucleotide and polynucleotide synthetic methods known in the art. For example, if rare mammalian codons are identified following purification of genomic DNA from the cell, rare mammalian codons are preferably edited to improve expression in the target cell. PCR-based amplification of one or more GV protein genes can be performed using appropriately designed primer pairs (e.g. using PrimerDesign or other programs known to those skilled in the art). PCR-based amplification can be followed by ligation (e.g. using T4 DNA ligase) of a polynucleotide encoding gas vesicle gene amplicon into an appropriate construct in a plasmid suitable for propagation in bacteria or archaea, such as transformation-competent E. coli DH5alpha or other competent E. coli type, followed by growth of transformed cell cultures, purification of the plasmid for confirmation of the cloned gene by DNA sequence analysis, among other methods known to those skilled in the art. Expression vectors can comprise plasmid DNA, viral vectors, or non-viral vectors, among others known to those skilled in the art, comprising appropriate regulatory elements such as promoters, enhancers, and post-transcriptional and post-translational regulatory sequences that are compatible with the mammalian cell intended to heterologously express the GV, as would be understood by a skilled person. In particular, in embodiments described herein, expression vectors suitable for regulating heterologous expression of GVs comprise those having promoters and other regulatory elements known to skilled persons that are compatible with mammalian cells, including cell lines, primary cells cultured in vitro such as petri dishes or introduce the GV gene circuits inside the animal to genetically engineer cells directly inside the animal and described above. Promoters can be constitutively active or inducible (and chosen to be selectively expressed in different cell types).

In particular, in some embodiments described herein, production of a GV gene sequences can be codon-optimized (for example to remove rare mammalian codons) for expression in the mammalian cell type according to methods identifiable by a skilled person. As would be understood by those skilled in the art, the term “codon optimization” as used herein refers to the introduction of synonymous mutations into codons of a protein-coding gene in order to improve protein expression in expression systems of a particular organism, such as human, in accordance with the codon usage bias of that organism. The term “codon usage bias” refers to differences in the frequency of occurrence of synonymous codons in coding DNA. The genetic codes of different organisms are often biased towards using one of the several codons that encode a given amino acid over others, and use the one codon with a greater frequency than expected by chance. Optimized codons in organisms reflect the composition of their respective genomic tRNA pool. The use of optimized codons can help to achieve faster translation rates and high accuracy (and ultimately higher recombinant protein yield).

In some embodiments, one or more statistical methods proposed and used to analyze codon usage bias the field of bioinformatics and computational biology can be used for codon optimization in the sense of the disclosure. Methods such as the ‘frequency of optimal codons’ (Fop), the Relative Codon Adaptation (RCA) or the ‘Codon Adaptation Index’ (CAI) are used to predict gene expression levels, while methods such as the ‘effective number of codons’ (Nc) and Shannon entropy from information theory are used to measure codon usage evenness. Multivariate statistical methods, such as correspondence analysis and principal component analysis, are widely used to analyze variations in codon usage among genes. There are many computer programs to implement the statistical analyses enumerated above, including CodonW, GCUA, INCA, and others identifiable by those skilled in the art. Several software packages are available online for codon optimization of gene sequences, including those offered by companies such as GenScript, EnCor Biotechnology, Integrated DNA Technologies, ThermoFisher Scientific, among others known those skilled in the art. Those packages can be used in providing GV proteins with codon usage ensuring optimized expression in various prokaryotic cell systems as will be understood by a skilled person. In particular, codon optimization in embodiments herein described can be used primarily to remove or limit the use of rare codons, or keep codon usage above ˜10%)

Mammalian cell used herein to include a GVES of the disclosure refers to a mammalian cell which can be transduced, infected, transfected or transformed with a vector under certain culture conditions. The vector can be plasmid, a viral particle, or others identifiable to a person skilled in the art. The term mammalian cell refers to cells isolated from an animal (mammal) tissue and expanded in culture for use as therapeutic and research tools.

In some embodiments, the transformed mammalian cells can comprise one or more cells such as T-cells, hematopoietic stem cells, mesenchymal stem cells, neural precursor cells, macrophages, fibroblasts or cardiomyocytes and any cell where one can express reporter genes (e.g. Green fluorescent protein (GFP)).

In some embodiments the Stoichiometric GVES of the disclosure can be used in connection with CAR-T and/or CAR-P cells, in particular in connection with the diagnostic and/or therapeutic applications.

As used herein, the term “chimeric antigen receptor” (CAR) refers to a synthetic, modular receptor engineered to endow a host cell—typically an immune cell such as a T lymphocyte, natural killer (NK) cell, or macrophage—with the ability to recognize a defined antigen in a manner independent of the endogenous major histocompatibility complex (MHC). A CAR-T cell is a T lymphocyte genetically modified to express such a receptor, while a CAR-P cell refers to a chimeric antigen receptor phagocyte, typically derived from a macrophage, monocyte, or dendritic-cell lineage engineered to perform antigen-specific phagocytosis, secretion, or immunomodulation.

A CAR construct generally comprises: (i) an extracellular antigen-binding domain, frequently a single-chain variable fragment (scFv) derived from a monoclonal antibody, nanobody, or ligand; (ii) a hinge or spacer region providing flexibility and optimal receptor geometry; (iii) a transmembrane domain anchoring the receptor in the cell membrane; and (iv) one or more intracellular signaling domains that trigger cellular activation. For CAR-T cells, the signaling domain typically includes a CD35 chain fused to one or more co-stimulatory motifs such as CD28, 4-1BB (CD137), OX40, or ICOS, thereby controlling the intensity, persistence, and cytokine profile of the immune response. CAR-P receptors employ analogous architectures but couple the antigen-recognition domain to phagocytic signaling motifs such as those from Megf10, FcRγ, or CD3 to induce engulfment rather than cytolytic activity.

CAR-T and CAR-P cells can be generated ex vivo by transducing isolated immune cells with viral or non-viral vectors carrying a CAR expression cassette under a constitutive or inducible promoter. The engineered cells are then expanded and re-introduced into the subject, where they perform antigen-specific recognition and effector functions. Expression of a CAR can be combined with gas vesicle expression systems (GVES) or other reporter modules described herein to enable real-time imaging of CAR-cell localization, viability, and activation.

CAR-T cells have demonstrated clinical efficacy in the treatment of hematologic malignancies, including B-cell acute lymphoblastic leukemia, diffuse large B-cell lymphoma, and multiple myeloma, by targeting cell-surface antigens such as CD19, CD20, CD22, BCMA, and CD138. Additional constructs are in development for solid tumors, targeting antigens including HER2, EGFR, GD2, mesothelin, MUC1, and PSMA. CAR-P systems are particularly suited for therapeutic applications requiring phagocytosis or antigen presentation, such as clearance of tumor cells, amyloid plaques, or infected cells, and for re-education of the tumor microenvironment via secretion of cytokines or checkpoint modulators (e.g., IL-12, IL-15, or PD-L1 inhibitors).

Further therapeutic embodiments include dual-CAR or tandem-CAR architectures, in which a single cell expresses two or more CARs recognizing different antigens to enhance selectivity or prevent tumor escape; switchable CARs activated by small molecules or bispecific adaptors; and safety-switch CARs containing suicide genes or protease-cleavable inactivation domains. CAR-P macrophages may additionally be engineered with synthetic receptors that recognize pathogen-associated molecular patterns (PAMPs) or damage-associated molecular patterns (DAMPs) to enable antimicrobial or anti-inflammatory therapy.

In certain embodiments, CAR-T or CAR-P cells are employed as diagnostic or reporter cells to detect the presence, distribution, or activity of a specific antigen in vivo. When coupled with genetically encoded reporters such as the gas vesicle constructs (GVES, GVPC, or GVRMC) described herein, the engineered immune cells can be non-invasively visualized using ultrasound or magnetic resonance imaging (MRI). Such CAR-reporter hybrids allow monitoring of cell trafficking, tumor infiltration, persistence, or activation state within a subject over time.

Additional diagnostic embodiments include the use of CAR-P cells as antigen-capturing biosensors within implantable or in vitro platforms, where antigen engagement triggers phagocytosis, secretion of detectable metabolites, or expression of imaging reporters. CAR-T or CAR-P cells engineered with inducible GV expression can therefore provide dual-function therapeutic and diagnostic (“theranostic”) capabilities, enabling real-time assessment of therapeutic efficacy and immune dynamics within target tissues.

Accordingly, CAR-T and CAR-P technologies, alone or in combination with gas-vesicle reporter constructs described herein, can be configured for therapeutic, diagnostic, or integrated theranostic use. The combination of targeted immune activation and non-invasive imaging enables precision monitoring of engineered immune-cell behavior and facilitates adaptive therapeutic control. Such embodiments include GV-expressing CAR-T cells that report antigen encounter via ultrasound contrast enhancement or MRI phase-shift, CAR-P macrophages designed to visualize phagocytic activity in inflamed or neoplastic tissue, and multiplexed CAR-reporter cell systems used to stratify patient response or guide combination immunotherapies.

In some embodiments, the transformed mammalian cells can be part of a tissue in vivo or ex vivo.

In some embodiments, the transformed mammalian cells can be isolated mammalian cells such as mammalian cell lines. Mammalian cell lines used herein refer to human or non-human mammalian recombinant expression systems capable of producing post-translational modifications which closely resemble those in mammalian cells in vivo. Exemplary non-human mammalian cell lines include CHO-K1, mouse myeloma cell lines such as NS0, SP2/0, rat myeloma cell lines such as YB2/0, baby hamster kidney (BHK), N2A cells, HeLa, Jurkat, NIH3T3, and others identifiable to a person skilled in the art. Human mammalian cell lines are immortalized cells propagated in vitro from primary explants of human tissue or body fluid. Exemplary human cell lines include HEK293 and its derivatives, HT-1080, PER.C6, Huh-7 as well as others identifiable to a person skilled in the art.

In some embodiments, the transformation can occur in an individual of a mammalian species such as Homo sapiens or Mus musculus, for example, among others. In some embodiments, mammalian cells in the sense of the disclosure comprise stem cells, progenitor cells, induced pluripotent stem cells, and others identifiable by a skilled person.

In some embodiments herein described, the GVES herein described can be introduced in a mammalian cell to provide a reportable molecular component (herein GVRMC) of a gas vesicle reporting (GVR) genetic circuit in operative connection with other molecular components of the genetic circuit to report occurrence of a biochemical event in the mammalian cell.

The term “molecular component” as used in connection with the GVR genetic circuits described herein indicates a chemical compound or a structure comprised of a plurality of chemical compounds comprised in a cellular environment. Exemplary molecular components thus comprise polynucleotides, such as ribonucleic acids or deoxyribonucleic acids, polypeptides, polysaccharides, lipids, amino acids, peptides, sugars and/or other small or large molecules and/or polymers that can be found in a cellular environment. In some embodiments described herein, a molecular component of a GVR genetic circuit is a GV type or a cluster thereof.

The term “genetic molecular component” as used herein indicates a molecular unit formed by a gene (possibly comprising or formed by a cluster of genes), an RNA transcribed from the gene or a portion thereof and optionally a polypeptide or a protein translated from the transcribed RNA. In genetic circuits herein described, the biochemical reactions connecting the genetic molecular component to another molecular component of the circuit can involve any one of the gene, the transcribed RNA and/or the polypeptide forming the molecular component.

A gene comprised in a genetic molecular component is a polynucleotide that can be transcribed to provide an RNA and typically comprises coding regions as well as one or more regulatory sequence regions, which is a segment of a nucleic acid molecule which is capable of increasing or decreasing transcription or translation of the gene within an organism either in vitro or in vivo. In particular, coding regions of a gene herein described can comprise one or more protein coding regions which when transcribed and translated produce a polypeptide, or if an RNA is the final product only a functional RNA sequence that is not meant to be translated. Regulatory regions of a gene herein described comprise promoters, transcription factor binding sites, operators, activator binding sites, repressor binding sites, enhancers, protein-protein binding domains, RNA binding domains, DNA binding domains, silencers, insulators and additional regulatory regions that can alter gene expression in response to stimuli as will be recognized by a person skilled in the art.

An RNA of a genetic molecular component comprises any RNA that can be transcribed from a gene, such as a messenger ribonucleic acid (mRNA), short interfering ribonucleic acid, or ribonucleic acid capable of acting as a regulating factor in the cell. mRNA comprised in a genetic molecular component comprises regions coding for the protein as well as regulatory regions. mRNA can have additional control elements encoded, such as riboregulator sequences or a protein binding aptamer sequence placed upstream of the gene so the protein blocks ribosomes and conditionally prevents translation. Other RNAs that serve regulatory roles that can comprise the genetic molecular component include riboswitches, aptamers (e.g. malachite green, Spinach), aptazymes, guide CRISPR RNAs, and other RNAs known to those skilled in the art.

A protein comprised in a molecular component can be proteins with activating, inhibiting, binding, converting, or reporting functions. Proteins that have activating or inhibiting functions typically act on operator sites encoded on DNA, but can also act on other molecular components. Proteins that have binding functions typically act on other proteins, but can also act on other molecular components. Proteins that have converting functions typically act on small molecules, and convert small molecules from one small molecule to another by conducting a chemical or enzymatic reaction. Proteins with converting functions can also act on other molecular components. Proteins with reporting functions have the ability to be easily detectable by commonly used detection methods (absorbance, fluorescence, for example), or otherwise cause a reaction on another molecular component that causes easy detection by a secondary assay (e.g. adjusts the level of a metabolite that can then be assayed for). The activating, inhibiting binding, converting, or reporting functions of a protein typically form the interactions between genetic components of a genetic circuit. Exemplary proteins that can be comprised in a genetic molecular component comprise monomeric proteins and multimeric proteins, proteins with tertiary or quaternary structure, proteins with linkers, proteins with non-natural amino acids, proteins with different binding domains, and other proteins known to those skilled in the art.

The term “cellular molecular component” indicates a molecular component not encoded by a gene, or indicates a molecular component transcribed and/or translated by a gene but comprised in the circuit without the corresponding gene. Exemplary cellular components comprise polynucleotides, polypeptides, polysaccharides, small molecules and additional chemical compounds that are present in a cellular environment and are identifiable by a skilled person. Polysaccharides, small molecules, and additional chemical compounds can include, for example, NAD, FAD, ATP, GTP, CTP, TTP, AMP, GMP, ADP, GDP, Vitamin B1, B12, citric acid, glucose, pyruvate, 3-phosphoglyceric acid, phosphoenolpyruvate, amino acids, PEG-8000, FiColl 400, spermidine, DTT, b-mercaptoethanol maltose, maltodextrin, fructose, HEPES, Tris-Cl, acetic acid, aTc, IPTG, 3OC12HSL, 3OC6HSL, vanillin, malachite green, Spinach, succinate, tryptophan, and others known to those skilled in the art. Polynucleotides can include RNA regulatory factors (small activating RNA, small interfering RNA), or “junk” decoy DNA that either saturates DNA-binding enzymes (such as exonuclease) or contains operator sites to sequester activator or repressor enzymes present in the system. Polypeptides can include those present in the genetic circuit but not produced by genetic components in the circuit, or those added to affect the molecular components of the circuit.

In embodiments of genetic circuits herein described, one or more molecular components is a recombinant molecular component that can be provided by genetic recombination (such as molecular cloning) and/or chemical synthesis to bring together molecules or related portions from multiple sources, thus creating molecular components that would not otherwise be found in a single source.

In a GVRMC of the disclosure, at least one gene expression cassette of the gene expression cassettes of the GVES of the disclosure comprises a gas vesicle reporting (GVR) target region configured to be activated and/or inhibited by a molecular component of a genetic circuit.

These additional (GVR) target region can include genetic elements that allow control over cellular behavior through various biochemical processes including transcriptional control, translational control, post-translational control and other control processes identifiable to a person skilled in the art.

In some embodiments, the transcriptional control elements can include constitutive promoters, repressor and/or activator sites, recombination sites, inducible and/or tissue-specific promoters, or cell fate regulators. The translational control elements can include RNAi, Riboregulators, RNA secondary structural motifs included in the GVES mRNA, or Ribosome-binding sites. The post-translational control elements can include elements controlling phosphorylation cascades, protein receptor design, protein degradation element, and localization signals. Examples of these regulatory regions and their functional purposes can be found in published review articles such as Purnick et al. ([31]) (for example Table 1 of Purnick) as will be understood by a person skilled in the art.

In embodiments herein described, a genetic circuit comprises at least one genetic molecular component or at least two genetic molecular components, and possibly one or more cellular molecular components, connected one to another in accordance with a circuit design by activating, inhibiting, binding or converting reactions to form a fully connected network of interacting components.

In embodiments of the GVR genetic circuits described herein, the molecular components are connected with one another according to a circuit design in which a molecular component is an input and another molecular component is an output. In particular, a genetic circuit typically has one or more input or start molecular component which activates, inhibits, binds and/or convert another molecular component, one or more output or end molecular component which are activated, inhibited, bound and/or converted by another molecular component, and intermediary molecular components each inhibiting, binding and/or converting another molecular component and being activated, inhibited, bound and/or converted by another molecular component. In some embodiments of the genetic circuits herein described, the input is the biochemical event and/or a trigger molecular component and the output is activation of expression of a GV gene cluster and assembly of a GV type through binding reactions between gvps of the GV type. In other embodiments of the genetic circuits herein described, the input is a biochemical event and/or a trigger molecular component and the output is an intracellular spatial translocation of the GV type, the intracellular spatial translocation occurring typically through one or more converting and/or binding reactions as described herein. The output of GVR circuit herein described can be detected with ultrasound contrast, MRI SWI, light scattering and additional techniques to detect GV identifiable by a skilled person upon reading of the present disclosure.

The term “activating” as used herein in connection with a molecular component of a genetic circuit refers to a reaction involving the molecular component which results in an increased presence of the molecular component in the cellular environment. For example, activation of a genetic molecular component indicates one or more reactions involving the gene, RNA and/or protein of the genetic molecular component resulting in an increased presence of the gene, RNA and/or protein of the genetic molecular component (e.g. by increased expression of the gene of the molecular component, and/or an increased translation of the RNA). An example of “activating” described herein comprises the initiation of expression of a GV gene cluster under the control of the tetracycline-inducible promoter (using reverse tetracycline-controlled transactivator) followed by the ultrasound response of mammalian ARGs (e.g., see Example 13, and 18).

Activation of a molecular component of a genetic circuit by another molecular component of the circuit can be performed by direct or indirect reaction of the molecular components. Examples of a direct activation of a genetic molecular component comprise in a circuit the production of an alternate sigma factor (molecular component of the circuit) that drives the expression of a gene controlled by the alternate sigma factor promoter (other molecular component of the circuit), or the production of a small ribonucleic acid (molecular component of the circuit) that increases expression of a riboregulator-controlled RNA (molecular component of the circuit). Examples of indirect activation of a genetic molecular component comprise the production of a first protein that inhibits an intermediate transcriptional repressor protein, wherein the intermediate transcriptional repressor protein represses the production of a target gene, such that the first protein indirectly activates expression of the target gene.

The term “inhibiting” as used herein in connection with a molecular component of a genetic circuit refers to a reaction involving the molecular component of the genetic circuit and resulting in a decreased presence of the molecular component in the cellular environment. For example, inhibition of a genetic molecular component indicates one or more reactions involving the gene, RNA and/or protein of the genetic molecular component resulting in a decreased presence of the gene, RNA and/or protein (e.g. by decreased expression of the gene of the molecular component, and/or a decreased translation of the RNA). Inhibition of a cellular molecular component indicates one or more reactions resulting in a decreased production or increased conversion, sequestration or degradation of the cellular molecular components (e.g. a polysaccharide or a metabolite) in the cellular environment.

Inhibition can be performed in the genetic circuit by direct reaction of a molecular component of the genetic circuit with another molecular component of the circuit or indirectly by reaction of products of a reaction of the molecular components of the genetic circuit with the another molecular component of the circuit.

The term “binding” as used herein in connection with molecular components of a genetic circuit refers to the connecting or uniting two or more molecular components of the circuit by a bond, link, force or tie in order to keep two or more molecular components together, which encompasses either direct or indirect binding where, for example, a first molecular component is directly bound to a second molecular component, or one or more intermediate molecules are disposed between the first molecular component and the second molecular component another molecular component of the circuit. Exemplary bonds comprise covalent bond, ionic bond, van der Waals interactions and other bonds identifiable by a skilled person.

In some embodiments, the binding can be direct, such as the production of a polypeptide scaffold that directly binds to a scaffold-binding element of a protein. In other embodiments, the binding may be indirect, such as the co-localization of multiple protein elements on one scaffold. In some instances binding of a molecular component with another molecular component can result in sequestering the molecular component, thus providing a type of inhibition of said molecular component. In some instances, binding of a molecular component with another molecular component can change the activity or function of the molecular component, as in the case of allosteric interactions between proteins, thus providing a type of activation or inhibition of the bound component.

The term “converting” as used herein in connection with a molecular component of the circuit refers to the direct or indirect conversion of the molecular component into another molecular component. An example of this is the conversion of chemical X by protein A to chemical Y that is then further converted by protein B to chemical Z.

In the GVR genetic circuits in the sense of the present disclosure, the gvp genes and related cassettes included with a GVES of the disclosure are introduced into a mammalian cell to provide a reportable molecular component connected with other genetic or cellular molecular components according to a circuit design, wherein the GV type is expressed or the GV type is intracellularly spatially translocated when the GVGC genetic circuit operates according to the circuit design in response to a biochemical event and/or to a trigger molecular component.

The term “reportable molecular component” as used herein indicates a molecular component capable of detection in one or more systems and/or environments. The terms “detect” or “detection” as used herein indicates the determination of the existence, presence or fact of a target in a limited portion of space, including but not limited to a sample, a reaction mixture, a molecular complex and a substrate. The “detect” or “detection” as used herein can comprise determination of chemical and/or biological properties of the target, comprising ability to interact, and in particular bind other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified. In particular, in embodiments herein described detection of the reportable molecular component comprising a GV type is performed through contrast enhanced imaging techniques such as ultrasound and MRI (and light scattering).

The term “biochemical event” as used herein refers to an activating, inhibiting, binding or converting reaction between two or more molecular components within a prokaryotic cell.

Accordingly, in some embodiments, at least one genetic molecular component of the GVR genetic circuit comprises a GVB cassette and additional GVP cassettes of the GVES of the disclosure comprising genes gvpB gene gvpN, gvpF, gvpG, gvpL, gvpS, gvpK, gvpJ, and gvpU genes, in a gas vesicle (GV) gene cluster in which the GV genes are operatively connected to a promoter configured to be activated directly or indirectly by the biochemical event, and directly initiate expression of a GV type.

In some embodiments herein described, a genetic molecular component of the GVR genetic circuit comprises a gas vesicle (GV) gene cluster comprising the GVB cassette and additional GVP cassettes of the GVES of the disclosure in which genes gvpB gene gvpN, gvpF, gvpG, gvpL, gvpS, gvpK, gvpJ, and gvpU genes are configured to be activated directly or indirectly by the biochemical event, and directly initiate expression of a GV type through interactions with promoters as well as one or more enhancers and/or other regulatory DNA elements comprised within the GVB and/or additional GVP cassettes, which are identifiable by those skilled in the art. As would be understood by those skilled in the art, promoters are DNA regulatory elements that are typically located adjacent to the transcription start sites of genes, or a cluster of genes, on the same strand and upstream on a DNA sequence (towards the 5′ region of the sense strand), and for transcription to occur, the enzyme that synthesizes RNA, known as RNA polymerase, attaches to the promoter. Promoters contain DNA sequences identifiable by those skilled in the art, such as those that provide binding sites for RNA polymerase and also for proteins that function as transcription regulatory factors that can either activate or repress gene transcription.

The term “transcription regulatory factor” or “transcription factor” as used herein refers to any type of factors that can function by acting on a regulatory DNA element such as a promoter or enhancer sequence. The transcription regulatory factors can be broadly classified into a transcription repression factor (also referred to as “repressor”) and a transcription activation factor (also referred to as “activator”). The transcription repression factor acts on a regulatory DNA element to repress the transcription of a gene, thereby reducing the expression level of the gene. The transcription activation factor acts on a regulatory DNA element to promote the transcription of a gene, thereby increasing the expression level of the gene.

In particular, a transcription regulatory factor has typically at least one DNA-binding domain that can bind to a specific sequence of enhancer or promoter sequences. Some transcription factors bind to a DNA promoter sequence near the transcription start site and help form the transcription initiation complex. Other transcription factors bind to other regulatory sequences, such as enhancer sequences, and can either stimulate or repress transcription of the related gene.

Examples of specific transcription repression factors include KRAB, repressor domains of proteins Egr-1, Oct2A, Dr1, YY1, RE-1 silencing transcription factor (REST), Retinoblastoma protein, and MeCP2, mSin interaction domain, TALE repressors), and other identifiable by a skilled person, as well as homologues of known repression factors, that function in both prokarayotic and eukarayotic systems. Examples of transcription activation factors include (VP-16, VP-64, etc.) as well as homologues of known activation factors, that function in eukaryotic systems.

In some embodiments, one or more promoters operatively connected to one or more GVGC genes comprised within the GVB cassette and additional GVP cassettes of the GVES of the disclosure can be configured to be activated directly or indirectly by one or more biochemical events. In particular, in some embodiments, activation of expression of a GV genes introduced in a mammalian cell, can be linked to another molecular component in the GVR genetic circuit through activator or repressor transcription factors. In some embodiments, expression of the transcription factors can be regulated by a promoter of interest (see Examples section). In other embodiments, transcription factors can be regulated post-translationally through degradation or phosphorylation of the transcription factor.

Accordingly, the reportable genetic molecular component of the GVR genetic circuit comprising the GVB cassette and additional GVP cassettes of the GVES of the disclosure in which genes gvpB gene gvpN, gvpF, gvpG, gvpL, gvpS, gvpK, gvpJ, and gvpU genes are operatively connected to a promoter configured to be activated directly or indirectly by the biochemical event, and directly initiate expression of a GV type can in several embodiments comprise promoters and/or other DNA regulatory elements having one or more sequences identifiable to those skilled in the art that are configured to function as binding sites for any known transcription regulatory factor.

For example, in some embodiments GV genes expression in GVR circuit of the disclosure can be activated by promoters inducible by sugars (e.g., L-arabinose, L-rhamnose, xylose and sucrose), antibiotics (e.g., tetracycline), CRISPR-dCas9 (possibly in conjunction with conditionally active gRNAs), heat shock promoters, pH-dependent promoters, oxidation stress-dependent promoters, radiation-dependent promoters, metal-inducible promoters, inflammation factor-inducible promoters, signaling factor-inducible promoter and others identifiable by those skilled in the art. In other embodiments GV genes expression can be induced by activation of constitutive promoters of varying strengths that are suitable for regulating expression in mammalian cells described herein and identifiable by those skilled in the art.

In other embodiments, the GV gene or one or more of the regulatory elements of GVR circuit of the disclosure, is surrounded by recombination sites that are recognized by a recombinase, whose expression or activity is connected through the genetic circuit to a biochemical event in the bacterial cell. For example, a GV genes introduced in the mammalian cell in reverse (3′-5′) orientation to its promoter (in 5′-3′ orientation) can be flanked by recombination sites surrounding the GV genes, with the recombination sites configured to allow inversion of the hybrid GV gene cluster upon expression or activation of its respective recombinase, wherein upon recombination the hybrid GV gene is flipped into a 5′-3′ orientation to allow initiation of expression by the promoter. Suitable recombination systems for use in mammalian cells are identifiable by those skilled in the art, such as the piggy-bac integrase system, phiC31 and Bxb1 integrases, and the FLP/FRT or Cre/lox recombination systems, and additional systems identifiable by a skilled person.

In embodiments described herein, a GV gene cluster introduced by the GVES of the disclosure comprised in one or more genetic molecular components of the GVR genetic circuits described herein is configured to function as a set of reporter genes, which together encode proteins required for the formation of a GV type, such that expression of the GV type functions as a genetically-encoded reporter of the biochemical event in the mammalian cell comprising a GVR genetic circuit. As described herein, the reportable characteristics of the GV are such that the genetically-encoded GV can be used as a contrast agent, which, when used together with one or more contrast-enhanced imaging techniques described herein, functions as a genetically-encoded reporter in prokaryotic cells that have been genetically engineered to comprise one or more of the GVR genetic circuits described herein.

In particular, in exemplary embodiments described herein, all the GV genes of the cluster (e.g. gvpF, gvpG, gvpJ, gvpL, gvpK, gvpS, and gvpU and gvpA) enable GV formation. Therefore, if expression any one of these genes is regulated according to the design of a GVR genetic circuit as described herein then the expression of the GV type will be regulated accordingly.

In some embodiments, the GVR genetic circuits described herein can comprise a plurality of genetic molecular components that function as Boolean logical operators in genetic circuit designs known to those skilled in the art, such as those described in [32, 33]. As would be understood by persons skilled in the art, Boolean logic is a branch of algebra in which the values of the variables are the truth values ‘true’ and ‘false’, usually denoted by the digital logic terms ‘1’ and ‘0’ respectively. In contrast with elementary algebra where the values of the variables are numbers, and the main operations are addition and multiplication, the main operations of Boolean logic are the conjunction ‘AND’, the disjunction ‘OR’, and the negation ‘NOT’. As understood by those skilled in the art, it is thus a formalism for describing logical relations in the same way that ordinary algebra describes numeric relations.

Accordingly, the term “AND gate” refers to a digital logic gate that behaves according to the truth table shown in Table 3. A ‘true’ output (1) results only if both the inputs to the AND gate are ‘true’ (1). If neither or only one input to the AND gate is ‘true’ (1), a ‘false’ (0) output results. Therefore, the output is always 0 except when all the inputs are 1.

TABLE 3
‘AND gate’ truth table:

| | |
|---|---|
| Input | Output |

| A | B | A AND B |
|---|---|---|
| 0 | 0 | 0 |
| 0 | 1 | 0 |
| 1 | 0 | 0 |
| 1 | 1 | 1 |

In particular, the term “AND gate” as used herein refers to the logical relation between two genetic molecular components in a GVR genetic circuit, wherein inputs ‘A’ and ‘B’ in Table 3 are two biochemical events, and the output ‘A AND B’ in Table 3 is the production of a certain GV type.

For example, in some embodiments of an “AND gate” comprised in a GVR genetic circuit described herein, the GVR genetic circuit comprises a plurality of genetic molecular components wherein at least a first genetic molecular component comprises a first subset of genes from the GV gene cluster, and at least a second genetic molecular component comprises a second subset of genes from the GV gene cluster, wherein together the GV proteins expressed from the first and second genetic molecular components are configured to form a GV type. In these embodiments, activation of both the first AND second genetic molecular component is required for the output of the GV type in the genetic circuit when the genetic circuit operates according to the design of the genetic circuit. For example, the first and second genetic molecular components can comprise promoters that are activated by two or more biochemical events in the mammalian cell comprising the GVR genetic circuit.

In exemplary embodiments, any of gvpN, gvpF, gvpG, gvpJ, gvpL, gvpK, gvpS, and gvpU and gvpA of a GV gene cluster formed by genes gvpB gene gvpN, gvpF, gvpG, gvpL, gvpS, gvpK, gvpJ, and gvpU genes within the GVB cassette and additional GVP cassettes of the GVES of the disclosure can be split into at least a first and second genetic molecular component comprising at least a first and a second subset of these genes to form an AND gate.

In other embodiments of an “AND gate” comprised in a GVGC genetic circuit, two or more regulatory elements operatively connected to a GV gene cluster comprised in a genetic molecular component of a GVGC genetic circuit that is activated by biochemical events A AND B would result in the output of the GV type in the GVGC genetic circuit. For example, the promoter requires binding of two transcriptional activators for activation of the promoter. In Examples described herein (see the Methods section of the Examples), GV gene clusters of exemplary ARG1 and ARG2 and A2C constructs is driven by the T7 promoter that has a lac operator downstream of the promoter. The T7 RNA Polymerase is regulated by the araBAD promoter (inducible by L-arabinose). The lac operator is repressed by LacI (IPTG derepresses). Therefore only under conditions wherein both IPTG AND L-ara are present are GVs expressed.

The term “OR gate” refers to a digital logic gate that behaves according to the truth table shown in Table 4. A ‘true’ output (1) results if either of the inputs to the OR gate are ‘true’ (1).

TABLE 4
‘OR gate’ truth table:

| | |
|---|---|
| Input | Output |

| A | B | A OR B |
|---|---|---|
| 0 | 0 | 0 |
| 0 | 1 | 1 |
| 1 | 0 | 1 |
| 1 | 1 | 1 |

In particular, the term “OR gate” as used herein refers to the logical relation between two genetic molecular components in a GVGC genetic circuit, wherein inputs ‘A’ and ‘B’ in Table 3 are two biochemical events, and the output ‘A OR B’ in Table 3 is the production of a certain GV type.

For example, in some embodiments of an “OR gate” comprised in a GVGC genetic circuit described herein, a promoter operatively connected to a GV gene cluster comprised in a genetic molecular component of a GVGC genetic circuit that is activated by biochemical events A OR B would result in the output of the GV type in the GVGC genetic circuit. For example, the promoter is activated by binding of either of two different transcriptional activators.

In other embodiments, an OR gate can be achieved through the use of two consecutive promoters. In exemplary embodiments, both these promoters can be located directly upstream of the GV gene cluster or they can be independently located directly upstream of any one or more of gvpN, gvpF, gvpG, gvpJ, gvpL, gvpK, gvpS, or gvpU and gvpA genes.

In other embodiments, GV genes introduced in the mammalian cell with a GVES of the disclosure can be flanked by recombination sites that are recognized by a recombinase, whose expression or activity is, in turn, activated in response to a biochemical event in the mammalian cell. For example, in these embodiments, one input signal can activate the GV genes organized within a GV gene cluster while a constitutive promoter is positioned in the opposite direction of the gene cluster. The second input would drive a recombinase that flips the promoter so that GV genes can be expressed. Exemplary recombinase systems comprise the piggy-bac integrase system, phiC31 and Bxb1 integrases, and the FLP/FRT or Cre/lox recombination systems, and additional systems identifiable by a skilled person.

The term “Negated AND gate” or “NOT gate” refers to a digital logic gate that behaves according to the truth table shown in Table 5. A ‘true’ output (1) results if either of the inputs to the OR gate are ‘true’ (1).

TABLE 5
‘Negated AND gate’ or “NOT gate” truth table:

| | |
|---|---|
| Input | Output |

| A | B | A NOT B |
|---|---|---|
| 0 | 0 | 0 |
| 0 | 1 | 0 |
| 1 | 0 | 1 |
| 1 | 1 | 0 |

In particular, the term “Negated AND gate” or “NOT gate” as used herein refers to the logical relation between two genetic molecular components in a GVGC genetic circuit, wherein inputs ‘A’ and ‘B’ in Table 5 are two biochemical events, and the output ‘A OR B’ in Table 5 is the production of a certain GV type.

For example, in some embodiments of an “Negated AND gate” or a “NOT gate” comprised in a GVR genetic circuit described herein, the GVGC genetic circuit comprises a plurality of genetic molecular components wherein at least a first genetic molecular component comprises a GV gene cluster, and at least a second genetic molecular component comprises an CRISPR/Cas9 complex configured to inhibit expression of a gvp gene comprised in the GV gene cluster, e.g. a gvpA. In these embodiments, activation of expression and the first genetic molecular component and absence of activation (or repression) of the second genetic molecular component are both required for the output of a GV type in the genetic circuit when the genetic circuit operates according to the design of the genetic circuit. For example, the first and second genetic molecular components can comprise promoters that are activated or repressed by one or more biochemical events in the mammalian cell comprising the GVGC genetic circuit.

In embodiments of the genetic circuits herein described wherein the input is a biochemical event and the output is an intracellular spatial translocation of the GV type, the GV type is a molecular component of the genetic circuit and intracellular spatial translocation of the GV type can occur through one or more converting and/or binding reactions involving the GV type as described herein.

In some embodiments, in the GVR genetic circuit herein described, an expression of the GV type or an intracellular spatial translocation of the GV type occurs when the hybrid GVR genetic circuit operates according to the circuit design in response to a trigger molecular component within the target mammalian cell.

In some embodiments, the trigger molecular component is a molecular component that is capable of being natively produced in the target host in its naturally occurring form. In particular, the natively produced molecular component can be a genetic molecular component or a cellular molecular component.

Examples of natively produced genetic molecular component can be one or more RNA or protein natively encoded in the genome of the naturally occurring form of the mammalian host and natively expressed by the target mammalian host. Examples of cellular molecular components natively produced by the target host comprise metabolites of enzymatic reactions produced by enzymes that are natively expressed by the target mammalian host in its naturally occurring form.

In these embodiments, the GVR genetic circuit comprises a GV type when the GVR genetic circuit operates according to a circuit design in response to the presence of the natively produced molecular component in the target mammalian cell.

In particular, in these embodiments, expression of the GVR in the mammalian host does not require the introduction into the host of any genetic molecular components in addition to the genetic molecular components comprising the GVGC. In these embodiments, the promoter operatively connected to a hybrid GV gene cluster in the GVGC genetic molecular component is configured to be activated in response to molecular components capable of being natively produced by the host in its naturally occurring form, such as natively expressed transcription factors. Genetic molecular components that can be activated by native molecular components include response elements (activating transcription factor 4 response element, activator protein 1 response element, antioxidant response element, cAMP response element, enhancer binding protein response element, hypoxia response element, metal response element, NFAT response element, p53 response element, serum response element, Smad binding element, Xenobiotic response element); additional are identifiable by those skilled in the art. Natively produced proteins or RNAs natively encoded in the genome of a particular mammalian cell hosts comprise transcription factors (SP-1, AP-1, C/EBP, EGR1, HSF, ATF/CREB, GLII, HIF, c-Myc, Oct-1, p53, NF-1, STAT1) and lncRNAs (B2, roX1, roX2, Xist); additional are identifiable by those skilled in the art. Metabolites produced in biochemical reactions produced in the naturally occurring form of the mammalian host comprise cytokines such as chemokines, interferons (IFNy), interleukins (IL-2, IL-10), lymphokines (CSF1, CSF2, CSF3), and tumor necrosis factors (TNFa), as well as hormones (including endocrine, paracrine, autocrine, and intracrine hormones) and growth factors (BMP, EGF, ephrin, EPO, FGF); additional are identifiable by those skilled in the art.

Thus, in these embodiments, the target host mammalian cell is labeled with expression of a GV type, wherein expression of the GV type occurs in presence of the trigger molecular component that is capable of being natively produced in the target mammalian cell host in its naturally occurring form. In several embodiments described herein, one or more GVR genetic circuits can be introduced into one or more mammalian cell hosts according to genetic engineering methods described herein and known to those skilled in the art. Different cells expressing different GVs would be possible. The methods to introduce the GVES and related GVRMC are identifiable by a skilled person upon reading of the disclosure

In other embodiments, the trigger molecular component is a heterologous molecular component that is not capable of being natively produced in the target mammalian host in its naturally occurring form. In these embodiments, the GVGC genetic molecular component is not configured to express the GV type in presence of a molecular component that is capable of being natively produced in the target mammalian host in its naturally occurring form, but is instead configured to express the GV type in presence of one or more heterologous (non-natively produced) trigger molecular components e.g. by using cell type specific promoters, described above, and/or viral transduction which would be cell type specific.

In these embodiments, the trigger molecular component can be one or more heterologous molecular components comprising a heterologous genetic molecular component and/or a heterologous cellular molecular component.

In some embodiments, the heterologous genetic molecular component can comprise one or more protein- and/or RNA-encoding genes and/or regulatory elements such as promoters and/or enhancer elements that are not native to the target mammalian genome. In some embodiments, the heterologous genetic molecular component can be introduced into the target prokaryotic host in addition to the one or more genetic molecular components comprising the GVGC. The additional heterologous genetic molecular component can be a constitutively expressed or an inducible genetic molecular component.

In some embodiments, the heterologous cellular molecular component can comprise a molecular component that is naturally present in the environment comprising the target prokaryotic cell, such as a metabolite produced by a mammalian host comprising the target prokaryotic host cell, or it can be a molecular component that is not naturally present in the environment comprising the target prokaryotic host cell, and introduced into the prokaryotic host cell, such as a drug configured to activate expression of the heterologous genetic component.

Accordingly, the GVR circuit of the disclosure comprise a first GVES reporting molecular component, which is a GVES genetic molecular component comprising the GVB cassette and at least one second GVES reporting molecular component which is a GVES genetic molecular component comprising the additional GVP cassettes of the GVES of the disclosure. In GVR circuit of the disclosure the first GVES reporting molecular component and the at least one second GVES reporting molecular component are activated to trigger expression of GV genes gvpB gene gvpN, gvpF, gvpG, gvpL, gvpS, gvpK, gvpJ, and gvpU to provide the gas vesicle in the mammalian cell.

In some embodiments, the GVES genetic molecular component of a GVR circuit in a mammalian host according to the present disclosure comprises promoter and/or enhancer elements that are configured to be activated in response to the presence of a heterologous molecular component. In exemplary embodiments, the promoter is a constitutive promoter such as CMV). In other exemplary embodiments, the promoter is activated by a heterologous transcription factor that is encoded in a heterologous genetic molecular component introduced into the target mammalian host in addition to the GVGC genetic molecular component; in exemplary embodiments described herein, the GVGC genetic molecular component comprises a promoter controlled by heterologous transcription factors, for example, (tetracycline-dependent repressor fused to transactivation domain (VP16 domain), similarly LacI and LexA fusions to transactivators (e.g. VP16) and repressor domains (KRAB), ET-dependent macrolide-responsive promoter, dead-Cas9 fusion to transactivators and repressors, zinc-finger proteins fused to transactivators and repressors, transcription activator-like effectors fused to transactivators and repressors).

In some embodiments, the GVGC genetic molecular component comprises recombination sites (e.g. piggy-bac recombination sites) surrounding one or more gvp genes comprised in the GV gene cluster or one or more regulatory elements (e.g. promoter) wherein the one or more gvp genes or regulatory elements are introduced into a mammalian host cell in an orientation that prevents expression of the encoded GV type, e.g., the promoter is in reverse orientation relative to the GV gene cluster; in these embodiments a heterologous genetic molecular component comprising the recombinase enzymes required for flipping the orientation of the elements flanked by the recombinase sites in the GVGC genetic molecular component is also introduced into the prokaryotic host cell and expression of the GV type occurs upon recombinase-mediated flipping of the flanked elements in the GVGC genetic molecular component into an orientation allowing initiation of expression of the GV type.

In these embodiments, the GVR genetic circuit comprises a GV type is when the GVR genetic circuit operates according to a circuit design in response to the presence of the one or more heterologous molecular components in the target mammalian cell.

Thus, in these embodiments, the target mammalian host is labeled with expression of a GV type, wherein expression of the GV type occurs in presence of the heterologous trigger molecular component introduced into the target mammalian host.

Accordingly, in some embodiments, a method to provide a genetically engineered mammalian cell comprising one or more GVR genetic circuits is described. The method comprises genetically engineering a mammalian cell by introducing into the cell one or more GVR genetic circuits described herein.

The mammalian cells described herein can be genetically engineered using methods known to those skilled in the art. For example, one or more genetic molecular components of a GVR genetic circuit comprised in vectors described herein can be introduced into mammalian cells using transformation techniques such as lenti-virus, adeno associated virus, adenovirus, baculovirus, nanoparticles that contain genome editing enzymes such as CRISPR, TALENS, ZFNs, transposase and others known to those skilled in the art and described herein. In some embodiments, the genetic molecular components of a GVR genetic circuit are introduced into the mammalian cell to persist as a plasmid or integrate into the genome, following methods known in the art and described herein.

In embodiments herein described, the GVES system and related genetic circuits, cells, vectors, genetically engineered prokaryotic cells, compositions, methods and systems, in several embodiments can be used together with contrast-enhanced imaging techniques to detect and report a biological event the location of and/or biochemical events in genetically engineered mammalian cells in an imaging target site.

The term “contrast enhanced imaging” or “imaging”, as used herein indicates a visualization of a target site performed with the aid of a contrast agent present in the target site, wherein the contrast agent is configured to improve the visibility of structures or fluids by devices process and techniques suitable to provide a visual representation of a target site. Accordingly a contrast agent is a substance that enhances the contrast of structures or fluids within the target site, producing a higher contrast image for evaluation. In particular, as used herein, the term “contrast agent” refers to GVs expressed in prokaryotic cells comprised in the target site, the GVs comprised in GVGC genetic circuits in the mammalian cells when the GVGC genetic circuit operates according to a circuit design in response to a biochemical event, as described herein.

The term “target site” as used herein indicates an environment comprising one or more targets intended as a combination of structures and fluids to be contrasted, such as cells. In particular the term “target site” refers to biological environments such as cells, tissues, organs in vitro in vivo or ex vivo that contain at least one target. A target is a portion of the target site to be contrasted against the background (e.g. surrounding matter) of the target site. Accordingly, as used herein a target comprises one or more mammalian cells genetically engineered to comprise one or more GVGC genetic circuits as described herein within any suitable environment in vitro, in vivo or ex vivo as will be understood by a skilled person. Exemplary target sites include collections of microorganisms in vitro as well as cells grown in an in vitro culture, including, primary mammalian, cells, immortalized cell lines, tumor cells, stem cells, and the like. Additional exemplary target sites include tissues and organs in an ex vivo culture and tissue, organs, or organ systems in a subject, for example, lungs, brain, kidney, liver, heart, the central nervous system, the peripheral nervous system, the gastrointestinal system, the circulatory system, the immune system, the skeletal system, the sensory system, within a body of an individual and additional environments identifiable by a skilled person. The term “individual” or “subject” or “patient” as used herein in the context of imaging includes a single plant, fungus or animal and in particular higher plants or animals and in particular vertebrates such as mammals and more particularly human beings.

In some embodiments, imaging the target site comprising the mammalian host can be performed by applying ultrasound to obtain an ultrasound image of the target site.

The term “ultrasound imaging” or “ultrasound scanning” or “sonography” as used herein indicate imaging performed with techniques based on the application of ultrasound. Ultrasound refers to sound with frequencies higher than the audible limits of human beings, typically over 20 kHz. Ultrasound devices typically can range up to the gigahertz range of frequencies, with most medical ultrasound devices operating in the 1 to 18 MHz range. The amplitude of the waves relates to the intensity of the ultrasound, which in turn relates to the pressure created by the ultrasound waves. Applying ultrasound can be accomplished, for example, by sending strong, short electrical pulses to a piezoelectric transducer directed at the target. Ultrasound can be applied as a continuous wave, or as wave pulses as will be understood by a skilled person.

Accordingly, the wording “ultrasound imaging” as used herein refers in particular to the use of high frequency sound waves, typically broadband waves in the megahertz range, to image structures in the body. The image can be up to 3D with ultrasound. In particular, ultrasound imaging typically involves the use of a small transducer (probe) transmitting high-frequency sound waves to a target site and collecting the sounds that bounce back from the target site to provide the collected sound to a computer using sound waves to create an image of the target site. Ultrasound imaging allows detection of the function of moving structures in real-time. Ultrasound imaging works on the principle that different structures/fluids in the target site will attenuate and return sound differently depending on their composition. A contrast agent sometimes used with ultrasound imaging are microbubbles created by an agitated saline solution, which works due to the drop in density at the interface between the gas in the bubbles and the surrounding fluid, which creates a strong ultrasound reflection. Ultrasound imaging can be performed with conventional ultrasound techniques and devices displaying 2D images as well as three-dimensional (3-D) ultrasound that formats the sound wave data into 3-D images. In addition to 3D ultrasound imaging, ultrasound imaging also encompasses Doppler ultrasound imaging, which uses the Doppler Effect to measure and visualize movement, such as blood flow rates. Types of Doppler imaging includes continuous wave Doppler, where a continuous sinusoidal wave is used; pulsed wave Doppler, which uses pulsed waves transmitted at a constant repetition frequency, and color flow imaging, which uses the phase shift between pulses to determine velocity information which is given a false color (such as red=flow towards viewer and blue=flow away from viewer) superimposed on a grey-scale anatomical image. Ultrasound imaging can use linear or non-linear propagation depending on the signal level. Harmonic and harmonic transient ultrasound response imaging can be used for increased axial resolution, as harmonic waves are generated from non-linear distortions of the acoustic signal as the ultrasound waves insonate tissues in the body. Other ultrasound techniques and devices suitable to image a target site using ultrasound, such as non-linear ultrasound imaging such as AM, PI, AMPI, would be understood by a skilled person.

Types of ultrasound imaging of biological target sites include abdominal ultrasound, vascular ultrasound, obstetrical ultrasound, hysterosonography, pelvic ultrasound, renal ultrasound, thyroid ultrasound, testicular ultrasound, and pediatric ultrasound as well as additional ultrasound imaging as would be understood by a skilled person.

Applying ultrasound refers to sending ultrasound-range acoustic energy to a target. The sound energy produced by the piezoelectric transducer can be focused by beamforming, through transducer shape, lensing, or use of control pulses. The soundwave formed is transmitted to the body, then partially reflected or scattered by structures within a body; larger structures typically reflecting, and smaller structures typically scattering. The return sound energy reflected/scattered to the transducer vibrates the transducer and turns the return sound energy into electrical signals to be analyzed for imaging. The frequency and pressure of the input sound energy can be controlled and are selected based on the needs of the particular imaging task and, in some methods described herein, collapsing GVs. To create images, particularly 2D and 3D imaging, scanning techniques can be used where the ultrasound energy is applied in lines or slices which are composited into an image.

In some embodiments, the ultrasound imaging herein described can comprising collapsing a GV type expressed in the genetically engineered mammalian cell by applying collapsing ultrasound to the target site and/or imaging a GV type in the contrast agent by applying imaging ultrasound to the target site.

In some embodiments, a method is described to provide imaging of one or more biochemical events in a mammalian cell comprised in an imaging target site, the method comprising:
introducing into the mammalian cell a genetically engineered Gas Vesicle expression system (GVES) herein described in which the gvp genes encode for proteins of a Gas Vesicle (GV) type, wherein the GV type is a reportable molecular component of a gas vesicle reporting (GVR) genetic circuit, in which molecular components are connected one to another in accordance with a circuit design by activating, inhibiting, binding or converting reactions to form a fully connected network of interacting components, wherein in the GVR genetic circuit an expression of the GV type or an intracellular spatial translocation of the GV type occurs when the GVR genetic circuit operates according to the circuit design in response to the biochemical event
the introducing performed for a time and under condition to allow expression of the gvp genes and production of the GV type in the mammalian cell when the GVR genetic circuit operates according to the circuit design; and
imaging the target site comprising the mammalian host by applying an imaging ultrasound to the target site at a peak positive pressure below a collapse pressure of the GV type, increasing step-wise the peak positive pressure to above the collapse pressure of the GV type, taking image frames before, during, and after the step-wise increase, and performing signal separation on the image frames to image the GV type

In some embodiments, a method is described to label a target mammalian host, the method comprising:
introducing into the target mammalian host a genetically engineered Gas Vesicle expression system (GVES) herein described in which the gvp genes encode for proteins of a Gas Vesicle (GV) type, the introducing performed for a time and under condition to allow expression of the gvp genes and production of the GV type in the mammalian cell, wherein the GV type is a reportable molecular component of a gas vesicle reporting (GVR) genetic circuit, in which molecular components are connected one to another in accordance with a circuit design by activating, inhibiting, binding or converting reactions to form a fully connected network of interacting components, wherein in the GVR genetic circuit an expression of the GV type or an intracellular spatial translocation of the GV type occurs when the GVR genetic circuit operates according to the circuit design in response to a trigger molecular component within the target prokaryotic host;
   In the method, the introducing is performed under conditions resulting in presence of the trigger molecular component in the target mammalian host.
   In some embodiments, the method can further comprise imaging the target site comprising the target mammalian host, by imaging the target site comprising the mammalian host by applying an imaging ultrasound to the target site at a peak positive pressure below a collapse pressure of the GV type, increasing step-wise the peak positive pressure to above the collapse pressure of the GV type, taking image frames before, during, and after the step-wise increase, and performing signal separation on the image frames to image the GV type.

The ability of GVs to act as a contrast agent for both ultrasound allows them to act as an acoustomagnetic reporter, thus creating possibilities for multimodal imaging. In some embodiments herein described, when collapsing ultrasound is used in combination with MRI imaging, acoustically collapsing a GV type expressed in a mammalian cell can remotely in situ erase the GV type to enable a background-free magnetic resonance imaging of a target site. The background-free magnetic resonance imaging removes background noise posed by background contrast from endogenous sources [34, 35] by allowing GV types to be identified specifically based on their acoustic responses.

Accordingly, in various embodiments herein described imaging of a biochemical event and/or labeling of a mammalian cell can be performed by multiplex imaging as will be understood by a skilled person upon reading of the present disclosure.

In methods herein described, administration of one or more genetically engineered mammalian cell types comprising one or more GVR genetic circuits to a target site to be imaged, can be performed in any way suitable to deliver the one or more mammalian cells comprising a GVR genetic circuit to the target site to be imaged.

In some embodiments, in which the target site is the body of an individual or a part thereof, the one or more genetically engineered mammalian cell types comprising a GVR genetic circuit can be administered to the target site locally or systemically.

The wording “local administration” or “topic administration” as used herein indicates any route of administration by which the one or more genetically engineered bacterial cell types comprising a GVR genetic circuit is brought in contact with the body of the individual, so that the resulting location of the one or more genetically engineered bacterial cell types comprising a GVR genetic circuit in the body is topic (limited to a specific tissue, organ or other body part where the imaging is desired). Exemplary local administration routes include injection into a particular tissue by a needle, gavage into the gastrointestinal tract, and spreading a solution containing the one or more genetically engineered bacterial cell types comprising a GVR genetic circuit on a skin surface.

The wording “systemic administration” as used herein indicates any route of administration by which the one or more genetically engineered bacterial cell types comprising a GVR genetic circuit is brought in contact with the body of the individual, so that the resulting location of the one or more genetically engineered bacterial cell types comprising a GVR genetic circuit in the body is systemic (not limited to a specific tissue, organ or other body part where the imaging is desired). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

Accordingly, in some embodiments of methods herein described, administering the one or more genetically engineered mammalian cell types comprising a GVR genetic circuit can be performed topically or systemically by intradermal, intramuscular, intraperitoneal, intravenous, subcutaneous, intranasal, rectal, vaginal, and oral routes. In particular, the one or more genetically engineered mammalian cell types comprising a GVR genetic circuit can be administered by infusion or bolus injection, and can optionally be administered together with other biologically active agents. In some embodiments of methods herein described, administering the one or more genetically engineered mammalian cell types comprising a GVR genetic circuit can be performed by injecting the one or more genetically engineered mammalian cell types comprising a GVR genetic circuit such as in a body cavity or lumen. Upon expression of one or more GV types in one or more genetically engineered bacterial cell types comprised in the target site, the target site can be contrast imaged.

Accordingly, in some embodiments, a vector comprising one or more genetic molecular components of a GVR genetic circuit is described, wherein the vector is configured to introduce the one or more genetic molecular components comprised in a GVR genetic circuit into a mammalian cell.

The one or more genetic molecular components can be provided on or as one or more vectors configured for delivery into mammalian cells, including vectors suitable for diagnostic or therapeutic applications An expression vector is configured to carry and express the material in a cell under appropriate conditions. In some embodiments, a suitable vector can comprise a recombinant plasmid, a recombinant non-viral vector, or a recombinant viral vector.

In some embodiments the vectors can be selected from a variety of viral and non-viral systems. In some embodiments, viral vectors are used to achieve high-efficiency transduction, for example in ex vivo cell engineering or for in vivo delivery. Exemplary viral vectors include, but are not limited to, lentiviral vectors, which can facilitate stable genomic integration in both dividing and non-dividing cells and are commonly used in cell-based therapies such as CAR-T cell manufacturing. Other suitable viral vectors comprise adeno-associated viral (AAV) vectors, which are broadly used for in vivo gene therapy and can be selected from various serotypes to preferentially target specific tissues or organs for imaging or therapeutic intervention. Additional examples include gammaretroviral vectors, which integrate into dividing cells, adenoviral vectors for high-level transient expression, and herpes simplex virus (HSV) vectors, which can be engineered for applications targeting the nervous system. In other embodiments, non-viral vectors and delivery systems are used. These can include plasmid DNA or messenger RNA (mRNA) encoding the GVES components. Delivery of such non-viral nucleic acids can be accomplished using physical methods, such as electroporation or sonoporation, which are particularly suitable for ex vivo modification of therapeutic cells, or by carrier-based systems suitable for in vivo administration. Exemplary carrier systems include, without limitation, lipid nanoparticles (LNPs), liposomes (forming lipoplexes), and polymer-based carriers (forming polyplexes). For applications requiring stable, non-viral genomic integration, the GVES components may also be delivered as part of a transposon system, such as a piggyBac or Sleeping Beauty system. These vectors comprise the necessary regulatory elements, such as suitable promoters, enhancers, and terminators, for expression in the target mammalian cell, as are known to those skilled in the art

Vectors described herein can comprise suitable promoters, enhancers, post-transcriptional and post-translational elements for expression in mammalian that are identifiable by those skilled in the art. Vectors suitable for transduction of mammalian cells, are known to those skilled in the art. Exemplary vectors for transformation of a mammalian cell with genetic molecular components comprising GV gene clusters are described herein in the Examples.

Exemplary configurations and methods for using such vectors to deliver GV gene clusters are described herein in the Examples.

Accordingly, in some embodiments herein described, a genetically engineered mammalian cell and in particular a genetically engineered mammalian cell comprising one or more GVR genetic circuits is described.

In embodiments herein described, a composition is provided. The composition comprises one or more genetic molecular components of a GVR genetic circuit, vectors, or genetically engineered mammalian cells described herein together with a suitable vehicle.

The term “vehicle” as used herein indicates any of various media acting usually as solvents, carriers, binders or diluents for the one or more genetic molecular components, vectors, or prokaryotic cells herein described that are comprised in the composition as an active ingredient. In particular, the composition including the one or more genetic molecular components, vectors, or prokaryotic cells herein described can be used in one of the methods or systems herein described.

In some embodiments, the GVGC comprised in a genetic molecular component of a GVR genetic circuit can be engineered (e.g. by modifying the related gvp genes) to produce GV types with altered mechanical, acoustic, surface and targeting properties in order to achieve enhanced harmonic responses and multiplexed imaging to be better distinguished from background tissues. In particular, in some embodiments, a GV can be engineered to tune the related acoustic properties. In particular the engineering can be performed by genetically engineering a GV having an acoustic collapse pressure aP₀ performed to obtain a variant GV with a critical collapse pressure aP₁ lower than the aP₀.

In particular, in order to tune the acoustic collapse properties of the GV, one changes the structural proteins of the GV shell. For example, selecting proteins that make the GV shell longer, rounder, thicker, etc. or that add proteins to the shell that make it structurally stronger. Changes in the shape, size, and durability of the GV shell change its acoustic properties as will be understood by a skilled person.

Accordingly, in embodiments described herein, GVR genetic circuits comprising genetically-encoded GV types can be used together with contrast-enhanced imaging techniques such as ultrasound imaging and/or MRI to detect the location of and/or dynamic biochemical events in prokaryotic cells in an imaging target site, wherein the mammalian cells have been genetically engineered to comprise one or more GVR genetic circuits described herein. In some exemplary embodiments, this allows monitoring the activity of various natural and engineered signaling circuits in mammalian cells.

In some exemplary embodiments described herein, imaging of engineered mammalian cells expressing GV types in vivo allows imaging of engineered mammalian cells in target sites. However, conventional reporters based on fluorescent and luminescent proteins or radionuclide capture suffer from the poor penetration of light into tissue or the need to administer radioactive tracers [36-38]. In contrast to these techniques, ultrasound and MRI are widely available, inexpensive, radiation-free technologies capable of noninvasively imaging deep tissues [39]. For example, the spatial resolution of ultrasound is routinely on the order of 100 μm [40, 41] and can approach the single-micron level with recently developed super-resolution techniques [42]. With these performance characteristics and the ability to place signals within an anatomical context, ultrasound is an ideal technique for imaging microbes in vivo.

As described herein, GVESs and related polynucleotide constructs, GVR genetic circuits, vectors, genetically engineered mammalian cells, compositions, methods and systems can be used in several embodiments to detect biochemical events in mammalian cells In particular embodiments, the GVES and related genetic circuits, vectors, genetically engineered mammalian cells, compositions, methods and systems described herein enable cell imaging inside mammalian hosts.

In some embodiments described herein, GV type-expressing mammalian cells can be visualized in vivo in settings relevant to cell tracking such as immune cells, circulating tumor cells, stem cells, blood cells, or tracking of cellular parts around the body such as exosomes, differentiation of cells in stem cells and progenitor cells, genetic changes to cells, and additional settings identifiable by a skilled person. In exemplary embodiments described herein, expression of GV types can make mammalian cells visible to ultrasound at volumetric concentrations below 0.5%, allowing dynamic imaging of gene expression and other biochemical events, and allows the visualization in vivo, such as in tumor xenografts as shown in the Examples.

In some embodiments described herein, engineered gas vesicle gene clusters are used as reporter genes for ultrasound, giving this widely used noninvasive imaging modality the ability to visualize bacteria inside living animals with sub-100 μm resolution. In several embodiments described herein, transformation with GVES systems of the disclosure allow mammalian cells to be detected at concentrations above 3 mammalian cells per ultrasound voxel, making this technology relevant to a broad range of studies, demonstrating the ability of GVGC-expressing mammalian cells to be detected within living animals at relevant concentrations.

In some embodiments, the GVs and variants thereof comprised in GVR genetic circuits described herein can be used as a contrast agent in the contrast-enhanced imaging methods herein described.

In particular, a combination of different GV types and/or variants thereof comprised in GVR genetic circuits can be used as contrast agents, each expressed GV exhibiting a different acoustic collapse profile with progressively decreased midpoint collapse pressure values. In some cases, the percentage difference between the midpoint collapse pressure values of any given two expressed GVs types is at least twenty percent.

As mentioned above, the GV gene cluster and related GVR circuit, molecular component, polynucleotidic constructs, vectors, cells and compositions herein described can be provided as a part of systems to perform any of the above mentioned methods. The systems can be provided in the form of kits of parts. In a kit of parts, one or more the hybrid GV gene cluster and related GVR circuit, molecular component, polynucleotidic constructs, vectors, cells and other reagents to perform the methods herein described are comprised in the kit independently. The hybrid GV gene cluster and related GVR circuit, molecular component, polynucleotidic constructs, vectors, cells can be included in one or more compositions, and each the hybrid GV gene cluster and related GVR circuit, molecular component, polynucleotidic construct, vector and cell is in a composition together with a suitable vehicle.

In particular, the components of the kit can be provided, with suitable instructions and other necessary reagents, in order to perform the methods here disclosed. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (such as. wash buffers and the like).

The genetically engineered GVES, and related genetic circuits, vectors, genetically engineered mammalian cells, compositions, methods and systems herein described can be used in several embodiments to provide magnetic resonance imaging with enhanced contrast and molecular sensitivity at sub-nanomolar concentration.

The genetically engineered GVES, and related genetic circuits, vectors, genetically engineered mammalian cells, compositions, methods and systems herein described can be used in connection with various applications wherein contrast-enhanced imaging of a target site is desired. For example, the genetically engineered GVES, and related genetic circuits, vectors, genetically engineered mammalian cells, compositions, methods and systems herein described can be used for visualization of mammalian cells as part or introduced into a mammalian host, such as mammalian hosts, facilitating for example the study of the mammalian microbiome and the development of diagnostic and therapeutic prokaryotic cellular agents, among other advantages identifiable by a skilled person, in medical applications, as well diagnostics applications. Additional exemplary applications include uses of the genetically engineered GVES, and related genetic circuits, vectors, genetically engineered mammalian cells, compositions, methods and systems herein described in several fields including basic biology research, applied biology, bio-engineering, bio-energy, medical research, medical diagnostics, therapeutics, and in additional fields identifiable by a skilled person upon reading of the present disclosure.

Exemplary Imaging Methods and Applications Enabled by Stoichiometric Gas Vesicle Expression Systems can use gas of the disclosure as versatile contrast agents for both ultrasound and magnetic resonance imaging (MRI) by interacting with sound waves and specific gases.

Accordingly, the genetically engineered Gas Vesicle Expression Systems (GVES), Gas Vesicle Polynucleotide Constructs (GVPC), Gas Vesicle Reporting Molecular Components (GVRMC), and Gas Vesicle Reporting Genetic Circuits (GVRGC) provided herein can be utilized in conjunction with various contrast-enhanced imaging techniques, particularly ultrasound, to enable the detection, localization, and functional characterization of engineered mammalian cells in vitro or in vivo. The stoichiometric optimization and multi-construct delivery strategies enhance signal strength and applicability, facilitating advanced imaging protocols.

In some embodiments, he GVES can be configured to express a defined Gas Vesicle (GV) type within a target mammalian cell population, thereby rendering these cells detectable by ultrasound. Introduction of the GVES or related constructs into the cells, under conditions permitting expression and GV formation, results in intracellular accumulation of the acoustic reporters. The target site containing these engineered cells can then be imaged using ultrasound modalities sensitive to GV contrast.

In some embodiments imaging methods and systems compring involves collapse-based imaging, exemplified by Burst Ultrasound Reconstruction with Signal Templates (BURST). This procedure comprises applying an initial low-pressure ultrasound pulse or sequence of pulses to acquire a baseline image, followed by one or more high-amplitude ultrasound pulses sufficient to exceed the collapse pressure threshold of the expressed GV type. The resulting irreversible collapse generates a strong, transient non-linear acoustic signal. Subsequent image frames acquired immediately after the collapse pulse are processed, often by subtracting pre-collapse or later post-collapse frames, or by using template-matching algorithms, to specifically isolate the signal originating from the collapsing GVs, thereby providing a high-contrast, high-sensitivity map of GV-expressing cells. Another approach utilizes non-collapse-based non-linear imaging. Techniques such as Amplitude Modulation Pulse Sequence (xAM) or harmonic imaging employ lower acoustic pressures that induce reversible buckling or oscillation of the GV shell without causing collapse. This non-linear mechanical response generates harmonic frequencies (e.g., second harmonic) or modulation products (e.g., in xAM) distinct from the linear scattering of surrounding tissues. Detecting these specific non-linear signal components allows for visualization of GV-containing cells. This method has the advantage of being non-destructive, potentially allowing for repeated or continuous monitoring of the same cell population over time. The magnitude of the detected signal, whether from collapse-based or non-linear methods, quantified for example as a Signal-to-Background Ratio (SBR), can be correlated under calibrated conditions to the concentration of expressing cells or the level of expression driven by an operably linked promoter.

In some embodiments, the engineered gas vesicle polynucleotide constructs and related genetic circuits, vectors, genetically engineered mammalian cells, hosts, compositions, methods and systems can be used in connection with multiplexed Imaging Using Distinguishable Gas Vesicle Types In those embodiments, the simultaneous detection and differentiation of multiple distinct cell populations or cellular states can be obtained within the same target site through multiplexed imaging. This is achieved by engineering cells to express different GV types possessing measurably distinct acoustic properties. Such distinguishable GV types can be generated by utilizing different Gas Vesicle Gene Clusters (GVGCs) sourced from various organisms, or more practically, by modifying specific GV proteins, particularly the GvpC protein, within a given GVGC framework. Variations in GvpC structure, such as truncation, deletion, or sequence modification, alter the mechanical stability of the GV shell. For collapse pressure multiplexing, one can express a first GV type with a standard GvpC (higher collapse pressure, Pc1) linked to a first biological event, and a second GV type with a modified or absent GvpC (lower collapse pressure, Pc2<Pc1) linked to a second event. Imaging then involves applying a sequence of collapse pulses: first, a pulse with pressure P such that Pc2<P<Pc1 selectively collapses and detects the second GV population (e.g., via BURST signal). Subsequently, a higher pressure pulse (P >Pc1) collapses and detects the remaining first GV population. Alternatively, for non-linear signature multiplexing, different GvpC configurations can yield distinct non-linear responses (e.g., different harmonic generation efficiencies or different amplitude modulation characteristics) when interrogated with non-collapse pressures. By analyzing the frequency content or modulation pattern of the received ultrasound echoes using appropriate pulse sequences and signal processing, signals originating from different co-localized GV types can be computationally separated and mapped, allowing simultaneous visualization of the multiple cell states or populations they represent.

In some embodiments the GVES and related engineered gas vesicle polynucleotide constructs and related genetic circuits, vectors, genetically engineered mammalian cells, hosts, compositions, methods and systems can be used in connection with dynamic Reporting of Biochemical Events via Genetic Circuit Integration: in those embodiments The GVES can serve as a reportable output module (GVRMC) integrated into engineered genetic circuits (GVRGCs) designed to sense specific intracellular or environmental biochemical events. In one configuration, the expression of the entire GVES, controlled by a specific promoter responsive to the event of interest (e.g., activation of a signaling pathway, presence of a metabolite, expression of a transcription factor), is switched ON or OFF. Detection of the resulting GV signal via ultrasound then directly reports the occurrence or status of the sensed event. More advanced reporting capabilities involve engineering the GV type itself to dynamically change its acoustic properties in response to circuit activity.

A particularly useful embodiment involves sensing protease activity. Here, the GvpC protein of the expressed GV type is engineered to incorporate a specific amino acid sequence recognized and cleaved by a target protease. The GVRGC can be designed such that the engineered GV type is expressed, for example, constitutively. When the target protease becomes active within the cell (which may itself be an output of the GVRGC or indicative of a cellular process like apoptosis, differentiation, or response to therapy), it cleaves the GvpC on the surface of the pre-formed GVs. This cleavage alters the mechanical properties of the GV shell, typically reducing its stability. Ultrasound imaging can then detect this change, for instance, as a decrease in the GV's collapse pressure threshold (allowing detection via pressure-differential imaging) or as an increase in its non-linear scattering under non-collapse pressures. This method provides a dynamic readout of the biochemical event (protease activity) by modulating the signal characteristics of an existing reporter population, offering a potentially ratiometric or state-change-based reporting mechanism that can be distinguished from simple changes in overall reporter expression levels. Such methods enable sophisticated, non-invasive visualization of molecular processes and cellular functional states within intact biological systems using ultrasound.

Accordingly provided herein are methods and systems for the detection, localization, and functional characterization of engineered mammalian cells in vitro or in vivo. A system for imaging comprises, in one embodiment, a population of mammalian cells genetically engineered with a Gas Vesicle Expression System (GVES), Gas Vesicle Polynucleotide Construct (GVPC), Gas Vesicle Reporting Molecular Component (GVRMC), or Gas Vesicle Reporting Genetic Circuit (GVRGC) as described herein, which results in the expression and formation of a Gas Vesicle (GV) type within said cells. The system further comprises an imaging device, such as an ultrasound system or a Magnetic Resonance Imaging (MRI) system, configured to detect signals originating from the expressed GV type within a target site containing the engineered cells. The stoichiometric optimization and multi-construct delivery strategies described herein provide for an enhanced signal, enabling robust detection and facilitating a variety of advanced imaging protocols.

In several embodiments, methods are provided for singleplex detection, wherein a GVES is configured to express a single, defined GV type to report cell presence, localization, or the activity of an operably linked promoter. A method for imaging comprises introducing the GVES or related constructs into a population of mammalian cells, maintaining the cells under conditions sufficient to permit expression of the gvp genes and assembly of the GV type, and subsequently imaging a target site containing said cells using an ultrasound modality sensitive to GV contrast.

In one such method, detection is achieved using linear differential ultrasound imaging. This procedure comprises the steps of: (a) acquiring a first ultrasound image of the target site using a standard linear pulse sequence, such as B-mode, at a pressure below the collapse threshold of the GV type, wherein the GVs produce acoustic scattering and appear as contrast; (b) applying one or more high-amplitude ultrasound pulses at a pressure sufficient to exceed the collapse pressure threshold of the GV type, thereby irreversibly collapsing the GVs; (c) acquiring a second ultrasound image of the target site using the same linear pulse sequence, wherein the contrast from the GVs is now absent; and (d) processing the first and second images, for example by digital subtraction of the second image from the first image, to generate a differential image that specifically isolates the signal originating from the GV-expressing cells.

In another method, detection is achieved using non-linear, non-destructive imaging. Such methods utilize lower acoustic pressures that induce reversible buckling or oscillation of the GV shell without causing irreversible collapse. This non-linear mechanical response, which is distinct from the predominantly linear response of surrounding biological tissues, generates detectable signal components such as harmonic frequencies or modulation products. A method for non-linear, non-destructive imaging, exemplified by Amplitude Modulation Pulse Sequence (xAM), comprises the steps of: (a) applying a pulse sequence to the target site comprising at least two pulses or pulse trains at different amplitudes, such as a low-amplitude pulse and a high-amplitude pulse, wherein both amplitudes are below the collapse threshold of the GV type; (b) receiving the resulting acoustic echo signals from the target site; and (c) processing the received signals, for example by weighted subtraction, to cancel the linear background signal from tissue and isolate the non-linear signal component originating from the GVs. An image is then generated from this isolated non-linear signal. This non-destructive method permits repeated or continuous monitoring of the same cell population.

In further embodiments, a detection is achieved using high-sensitivity, collapse-based imaging, exemplified by Burst Ultrasound Reconstruction with Signal Templates (BURST). This method relies on detecting the unique, transient acoustic signature generated at the moment of GV collapse. The procedure comprises the steps of: (a) applying one or more high-pressure “burst” ultrasound pulses to the target site, wherein the pressure is sufficient to exceed the collapse pressure threshold and irreversibly destroy the GVs; (b) acquiring the acoustic echo data during and immediately following the burst pulse; and (c) processing the acquired data using a signal template-matching algorithm, wherein the template is designed to specifically recognize the characteristic acoustic signature of a GV collapse event. This processing computationally unmixes the GV collapse signal from all background tissue signals, enabling the generation of a high-sensitivity, high-contrast image of the GV-expressing cells.

In other embodiments, the GVs produced by the GVES serve as reporters for Magnetic Resonance Imaging (MRI), particularly for hyperpolarized xenon-129 (129Xe) MRI. A method for MRI detection comprises the steps of: (a) introducing the mammalian cells expressing the GV type into a target site within the imaging field of an MRI scanner; (b) administering hyperpolarized 129 Xe gas to the subject or target site, allowing the xenon to dissolve in the surrounding medium and to transiently bind to or exchange with the interior of the GVs; (c) applying a specific radiofrequency (RF) pulse tuned to the resonance frequency of the 129Xe bound within the GVs, thereby saturating this bound population; and (d) acquiring an MRI signal from the dissolved 129 Xe pool in the surrounding medium. The chemical exchange saturation transfer (CEST) of the saturated xenon from the GVs to the dissolved pool causes a measurable reduction in the overall detectable 129Xe signal. This signal reduction is specific to the presence of the GVs and is used to generate a contrast image reporting the location of the engineered cells.

The methods and systems provided herein further enable multiplexed imaging to simultaneously detect and differentiate multiple cell populations or cellular states. This is achieved by configuring different cell populations to express distinct GV types with unique and distinguishable imaging properties, for example by modifying the GvpC protein to alter mechanical stability.

A method for multiplexed ultrasound imaging using differential collapse pressure comprises the steps of: (a) introducing at least a first cell population of engineered cells of the and a second cell population of engineered cells of the present disclosure into a target site, wherein the first population expresses a first GV type having a first collapse pressure (Pc1) and the second population expresses a second GV type having a second collapse pressure (Pc2), where Pc2 is less than Pc1; (b) applying a first sequence of imaging and collapse pulses, such as a BURST sequence, using an acoustic pressure (P_first) sufficient to collapse the second GV type but not the first (Pc2<P_first <Pc1), thereby generating an image specific to the second cell population; and (c) subsequently applying a second sequence of imaging and collapse pulses using an acoustic pressure (P_second) sufficient to collapse the first GV type (P_second >Pc1), thereby generating an image specific to the first cell population. The two resulting images can be overlaid or displayed to show the distinct spatial distribution of the two populations.

A method for multiplexed ultrasound imaging using differential non-linear signatures comprises introducing engineered cell populations of the present disclosure expressing GV types engineered to have different non-linear buckling or harmonic generation profiles. The method comprises applying non-destructive, non-linear pulse sequences and processing the received echo signals, for example by analyzing their harmonic content or amplitude modulation characteristics, to computationally separate and map the signals originating from each distinct GV type.

These methods can be further combined with GVRGCs for dynamic event reporting. For example, a method for reporting protease activity comprises introducing cells expressing a stoichiometric GVRGC of the disclosure that encodes a GV type wherein the GvpC protein comprises a protease cleavage site. Upon activation of the target protease, the GvpC is cleaved, altering the GV's acoustic properties, for example, by lowering its collapse pressure. This change can be detected using the aforementioned multiplexed collapse pressure imaging method, wherein the signal from the cell population “shifts” from being detectable at a high pressure to being detectable at a low pressure, thereby providing a dynamic readout of the biochemical event.

In some embodiments, multimodal imaging methods are provided which can use the A method for acousto-magnetic validation comprises: (a) performing a 129Xe CEST MRI method as described above to acquire a first MRI signal map indicative of GV presence; (b) applying a high-pressure ultrasound pulse to the target site, sufficient to collapse the GVs; and (c) repeating the 129 Xe CEST MRI method. The disappearance or significant reduction of the CEST signal in the second MRI map validates that the signal originated from the acoustically-sensitive GVs. Such combinations, for example of MRI and BURST, can be used for enhanced imaging and validation.

In some embodiments the imaging methods performed in connection with stoichiometric gas vesicle gene expression system, engineered gas vesicle polynucleotide constructs and related genetic circuits, vectors, genetically engineered mammalian cells, hosts, compositions, methods and systems of the disclosure can be used for non-invasive imaging and tracking of various primary mammalian cells. As used herein, primary cells refer to cells directly isolated from a mammalian tissue that retain their differentiated characteristics. The method comprises providing a population of primary cells. These primary cells are genetically engineered, typically ex vivo, by introducing the Gas Vesicle Expression System (GVES) or the set of GV polynucleotide constructs described herein. This introduction can be performed using suitable methods such as lentiviral transduction, electroporation, or lipid-based transfection.

The primary cells can be harvested from a wide range of mammalian tissues comprising hepatocytes, cardiomyocytes, neurons, astrocytes, microglia, fibroblasts, endothelial cells, keratinocytes, adipocytes, pancreatic B-cells, renal epithelial cells, and smooth or skeletal muscle cells. Following engineering, and optional ex vivo expansion, the GVES-expressing primary cells are administered to a mammalian host, often to a specific target site (e.g., injection into damaged heart tissue for cardiomyocytes, or into the brain for neurons) or systemically. Alternatively, the cells may be engineered in vivo by direct administration of a suitable vector delivery system to the target tissue. The target site is then imaged at one or more time points using an ultrasound system configured for Gas Vesicle (GV) detection, such as by employing BURST or xAM imaging methods, to non-invasively monitor the location, engraftment, persistence, and optionally the viability or function of the administered primary cells.

In a particularly preferred set of embodiments, methods are provided for imaging and tracking engineered immune cells, which are of significant interest for cell-based therapies. The method comprises providing a population of immune cells, which are cells of hematopoietic origin. These immune cells are genetically engineered, typically ex vivo, by introducing the GVES or set of GV polynucleotide constructs as described herein. The multi-vector approach, particularly the three-vector lentiviral system, is highly suitable for transducing these cells, which can be difficult to engineer, to achieve robust GV expression without significantly impairing critical cell functions like cytotoxicity.

The immune cells suitable for this method can be derived from various lineages. Lymphoid lineage cells that can be engineered include, but are not limited to, T lymphocytes (including CD4⁺ helper T cells, CD8⁺ cytotoxic T cells, γδ T cells, and regulatory T cells), B lymphocytes, and natural killer (NK) cells. Myeloid lineage cells that can be engineered include, but are not limited to, monocytes, macrophages, dendritic cells, neutrophils, eosinophils, basophils, and mast cells. The method is also applicable to precursor or progenitor forms of these cells, such as those derived from bone marrow or hematopoietic stem cells.

Following ex vivo engineering and expansion, the GVES-expressing immune cells are administered to a mammalian host, typically by systemic injection, to allow them to traffic to target sites such as tumors, sites of infection, or areas of inflammation. At desired time points, the host is imaged using a GV-sensitive ultrasound modality to non-invasively visualize the homing, accumulation, expansion, and spatial distribution of the therapeutic immune cells within the target tissue. As described elsewhere herein, the GVES can be linked to constitutive promoters for cell tracking or to activation-specific promoters (e.g., NFAT) to functionally report on immune cell activation.

In some embodiments, stoichiometric gas vesicle gene expression system, engineered gas vesicle polynucleotide constructs and related genetic circuits, vectors, genetically engineered mammalian cells, hosts, compositions, methods and systems can be used to perform specific methods and systems for the non-invasive imaging, tracking, and functional monitoring of genetically engineered immune cells, such as Chimeric Antigen Receptor (CAR) T cells (CAR-T) and Chimeric Antigen Receptor Phagocytes (CAR-P), including macrophages. These methods leverage the robust expression of the stoichiometric Gas Vesicle Expression System (GVES) as a genetically encoded reporter, creating integrated “theranostic” cells capable of both therapeutic action and non-invasive imaging.

In some embodiments, stoichiometric gas vesicle gene expression system, engineered gas vesicle polynucleotide constructs and related genetic circuits, vectors, genetically engineered mammalian cells, hosts, compositions, methods and systems can be used to provide a system for engineering theranostic CAR-T and CAR-P Cells:

A system for generating an imageable therapeutic cell comprises one or more polynucleotide constructs encoding a Chimeric Antigen Receptor (CAR) and the set of polynucleotide constructs of the GVES as described herein (e.g., a three-vector system comprising the gvpA/B module, the AF1 module, and the AF2 module). In one embodiment, a method for producing such cells comprises providing a population of primary mammalian immune cells (e.g., T cells or monocyte/macrophage progenitors isolated from a subject) ex vivo. These cells are then co-transduced with a vector set comprising a first vector encoding the CAR and the set of GVES vectors. For example, a population of T cells can be co-transduced with four lentiviral vectors: one encoding an anti-CD19 CAR, one encoding the gvpA/B module, one encoding the AF1 module, and one encoding the AF2 module.

In an alternative system, the CAR and one or more GVES modules are combined onto a single vector to reduce the number of required vectors. For example, a single vector can be engineered to comprise a first expression cassette encoding the CAR and a second expression cassette encoding the gvpA/B module. In another configuration, a single polycistronic cassette may be engineered to encode the CAR and a GVES module, separated by a self-cleaving 2A peptide or an IRES element. This CAR-GVES vector is then co-transduced with the remaining GVES module vectors (e.g., AF1 and AF2 vectors) to produce the theranostic cell. The engineered cells are then expanded ex vivo before being administered to a mammalian host.

In some embodiments, stoichiometric gas vesicle gene expression system, engineered gas vesicle polynucleotide constructs and related genetic circuits, vectors, genetically engineered mammalian cells, hosts, compositions, methods and systems of the disclosure can be used to perform methods for tracking CAR-T and CAR-P cell localization and persistence:

For example in some embodiments, method for non-invasively tracking the localization, biodistribution, and persistence of therapeutic immune cells in vivo is described. This method comprises engineering CAR-T or CAR-P cells as described above, wherein the GVES modules are placed under the control of a constitutive mammalian promoter, such as an EF1a or CMV promoter. The resulting engineered cells, which continually express the GV type, are administered to a mammalian host, for example by systemic intravenous injection. At one or more time points following administration, a target site in the host, such as a known tumor location, or other organs such as the spleen, liver, or lymph nodes, is imaged using an ultrasound system. The ultrasound system is configured to perform a GV-sensitive imaging method, such as non-destructive xAM or high-sensitivity BURST imaging as described herein. The presence, spatial distribution, and intensity of the resulting ultrasound signal within the target site provides a non-invasive, quantitative measure of the engineered cell trafficking, homing to the target tissue, and persistence over time. This method allows for monitoring the “delivery” phase of the cell therapy.

In some embodiments, stoichiometric gas vesicle gene expression system, engineered gas vesicle polynucleotide constructs and related genetic circuits, vectors, genetically engineered mammalian cells, hosts, compositions, methods and systems of the disclosure can be used to perform methods for functional Imaging of CAR-T cell activation:

In particular in some embodiments, methods for non-invasively imaging the in vivo functional activation of CAR-T cells is described, allowing therapeutic action to be distinguished from mere cell presence. This method comprises engineering CAR-T cells with two components: first, a vector encoding the CAR under a constitutive promoter, and second, a Gas Vesicle Reporting Genetic Circuit (GVRGC). In this GVRGC, the GVES modules are placed under the control of an activation-dependent promoter, such as a promoter containing NFAT (Nuclear Factor of Activated T-cells) response elements. These engineered CAR-T cells are administered to a mammalian host. When the CAR-T cells are circulating and inactive, the NFAT promoter remains off, and no GVs are produced. Upon engagement of the CAR with its target antigen (e.g., CD19 on a tumor cell), the resulting intracellular signaling cascade activates the NFAT transcription factor, which in turn binds to the response elements and drives expression of the GVES modules and subsequent GV formation. Therefore, a method for imaging CAR-T activation comprises administering such engineered cells and subsequently imaging the target site with ultrasound. The generation of an acoustic signal, detectable by BURST or xAM, is indicative of successful CAR-T cell antigen engagement and activation, providing a direct, non-invasive readout of in situ therapeutic activity.

In some embodiments, In some embodiments, stoichiometric gas vesicle gene expression system, engineered gas vesicle polynucleotide constructs and related genetic circuits, vectors, genetically engineered mammalian cells, hosts, compositions, methods and systems of the disclosure can be used to perform methods for functional imaging of CAR-P phagocytic activity:

Similarly, a method for non-invasively imaging the in vivo functional activity of CAR-P cells is provided. This method comprises engineering CAR-P cells (e.g., macrophages) with a constitutively expressed CAR construct designed to initiate phagocytosis, and a GVRGC. In this GVRGC, the GVES modules are operably linked to a promoter responsive to the signaling pathways activated upon phagocytic engagement, such as signaling downstream of an FcRy motif or other phagocytic receptors. The engineered CAR-P cells are administered to the host. When the CAR-P cells engage their target (e.g., a tumor cell, amyloid plaque, or pathogen) and initiate phagocytosis, the intracellular signaling activates the GVRGC promoter, leading to expression of the GV type. An imaging method comprises applying ultrasound to the target site, wherein the appearance of an acoustic signal is indicative of active phagocytosis by the CAR-P cells. This method enables the specific visualization of locations where the CAR-P cells are actively clearing their targets.

In certain embodiments, a theranostic system for generating an imageable therapeutic cell is provided. The system comprises multiple genetic components configured to be introduced into a primary immune cell, such as a T lymphocyte or a macrophage.

A first component of the system is at least one polynucleotide construct encoding a Chimeric Antigen Receptor (CAR). This CAR construct is configured for expression in the immune cell and typically encodes an extracellular antigen-binding domain, a hinge, a transmembrane domain, and one or more intracellular signaling domains. For a CAR-T cell system, these intracellular signaling domains are selected to initiate T-cell activation, and comprise domains such as a CD3 zeta chain, and optionally one or more co-stimulatory motifs such as CD28, 4-1BB, OX40, or ICOS. For a CAR-P cell system, the intracellular signaling domains are selected to initiate phagocytosis, and comprise motifs such as an FcRγ motif.

A second component of the system is a Gas Vesicle Reporting Genetic Circuit (GVRGC). This GVRGC comprises the stoichiometric Gas Vesicle Expression System (GVES) described herein, including the gvpA/B differentially expressed gene module, the AF1 gvp differentially expressed gene module, and the AF2 gvp differentially expressed gene module. In this functional reporting system, the GVES modules are operably linked to an activation-dependent promoter, thereby coupling GV expression to the activation state of the immune cell.

In a specific embodiment for CAR-T cells, the activation-dependent promoter is a promoter responsive to the intracellular signaling cascade initiated by the CAR signaling domains. An exemplary promoter is an NFAT-responsive promoter, which is strongly activated by the calcium-calcineurin-NFAT signaling pathway triggered upon T-cell receptor or CAR engagement with an antigen.

In a specific embodiment for CAR-P cells, the activation-dependent promoter is a promoter responsive to the signaling pathways activated upon phagocytic engagement, such as a promoter operably linked to response elements for transcription factors activated downstream of an FcRγ motif or other phagocytic receptors.

In a further preferred embodiment, the GVRGC is configured as a two-stage amplification circuit. In this configuration, the activation-dependent promoter (e.g., the NFAT promoter) is not operably linked directly to the GVES modules. Instead, this promoter is operably linked to a gene encoding a heterologous transactivator protein, such as the reverse tetracycline-controlled transactivator (rtTA). The GVES modules, in turn, are operably linked to a second promoter, such as a Tetracycline Response Element (TRE) promoter. This promoter is strongly activated by the rtTA transactivator protein only in the presence of an exogenous small molecule, such as doxycycline. This system configuration allows a potentially weak or transient signal from the activation-dependent promoter to be amplified into robust expression of the rtTA, which then drives high-level, doxycycline-gated expression of the GVES modules and subsequent gas vesicle formation.

In some embodiments methods for functional imaging of engineered immune cells are a provided and in particular, such as methods for non-invasively imaging the in vivo functional activation of therapeutic immune cells, such as CAR-T or CAR-P cells.

In some embodiments, a method for functional imaging of CAR-T cell activation comprises, first, providing a population of T cells engineered ex vivo with the system described above, comprising both a CAR construct and a GVRGC wherein the GVES is operably linked to a T-cell activation-dependent promoter (e.g., an NFAT-responsive promoter). Second, the method comprises administering the engineered CAR-T cells to a mammalian host, for example one bearing a tumor expressing the target antigen. Following administration, the CAR-T cells circulate and, upon encountering target antigen on a tumor cell, the CAR binds and the intracellular signaling domains initiate the T-cell activation cascade. This signaling cascade activates the corresponding transcription factors, such as NFAT, which then bind to the activation-dependent promoter within the GVRGC and drive the expression of the GVES modules, resulting in the assembly and formation of gas vesicles. Third, the method comprises imaging the target site in the host using an ultrasound or MRI system configured to detect the GV-specific signal. The generation of a new acoustic or magnetic resonance signal, or a significant increase in signal intensity compared to a baseline, at the target site is indicative of successful CAR-T cell antigen engagement and functional activation, providing a non-invasive readout of in situ therapeutic activity.

In some embodiments, a method for functional imaging of CAR-P cell activation comprises, first, providing a population of phagocytic cells (e.g., macrophages) engineered ex vivo with a CAR construct and a GVRGC wherein the GVES is operably linked to a phagocytosis-dependent promoter. Second, the method comprises administering the engineered CAR-P cells to a mammalian host. Upon engagement and phagocytosis of a target (e.g., a tumor cell or amyloid plaque), the intracellular signaling activates the GVRGC promoter, driving expression of the GVES modules and formation of gas vesicles. Third, the method comprises imaging the target site, wherein the appearance of a GV-specific signal is indicative of active phagocytosis by the CAR-P cells at that location.

It will be apparent to one of ordinary skill in the art, upon reading the present disclosure, that the methods, systems, and compositions described herein are broadly applicable to numerous applications beyond those specifically exemplified. The methods for engineering cells with the stoichiometric Gas Vesicle Expression System (GVES) and related constructs, and the subsequent imaging of said cells, can be adapted for a wide variety of additional primary mammalian cell types, cell lines, and engineered cells. Such cells include, without limitation, stem cells (such as induced pluripotent stem cells, embryonic stem cells, hematopoietic stem cells, and mesenchymal stem cells), progenitor cells, fibroblasts, hepatocytes, cardiomyocytes, neurons, glial cells, endothelial cells, and keratinocytes. These engineered cells can be utilized and imaged in diverse contexts, including in vitro assays for drug screening or developmental studies, ex vivo systems for monitoring bioreactors or engineered tissues, and in vivo applications within a mammalian host. The in vivo methods are not limited to tumor models but can be applied to the imaging of engineered cells in various organs, such as the brain, heart, liver, kidney, pancreas, spleen, and skin, and for the study of numerous biological processes or pathological conditions, including, but not limited to, inflammation, autoimmune disease, tissue regeneration, wound healing, metabolic disorders, and neurodegenerative disease. A person skilled in the art can, based on the teachings provided herein, readily identify and implement the necessary adaptations to protocols for cell engineering, vector delivery, and imaging parameters suitable for these additional applications, cell types, organs, and conditions.

Further details concerning the genetically engineered GVES, and related genetic circuits, engineered mammalian cells and methods of the present disclosure will become more apparent hereinafter from the following detailed disclosure of examples by way of illustration only with reference to an experimental section.

### EXAMPLES

The stoichiometric GV genes expression systems, polynucleotide constructs, and related genetic circuits, vectors, genetically engineered mammalian cells, compositions, methods and systems herein disclosed are further illustrated in the following examples, which are provided by way of illustration and are not intended to be limiting.

In particular, the following examples illustrate exemplary methods and protocols for providing and using polynucleotide constructs, and related genetic circuits, vectors, genetically engineered mammalian cells, compositions, methods and systems. A person skilled in the art will appreciate the applicability and the necessary modifications to adapt the features described in detail in the present section, to additional genetically engineered GVES, and related genetic circuits, vectors, genetically engineered mammalian cells, compositions, methods and systems according to embodiments of the present disclosure.

The following materials and method were used in the exemplary embodiments reported in this section unless otherwise specified.

Plasmid construction and molecular biology All constructs were made via KLD mutagenesis or Gibson Assembly using enzymes from New England BioLabs. All plasmids and their sources of genetic material are described in Table 5A. Constructs were cloned in NEB Turbo Competent E. coli (High Efficiency). Fluorescent reporters referred to in the text as GFP and BFP are mEGFP and mTagBFP2, respectively.

TABLE 5A
Plasmids and Constructs
Plasmid

pLV-TRE3G-GvpA-IRES-NJKFGWV-mEGFP
pLV-TRE3G-NJKFGWV-mEGFP
pLV-TRE3G-GvpA-IRES-BFP-minEF1a-rtTA
pLV-TRE3G-GWV-mEGFP
pLV-TRE3G-NJKF-minEf1a-rtTA
pLV-TRE3G-GvpA-IRES-BFP
PLV-TRE3G-NJKF-NFATx4-rtTA
pLV-TRE3G-mScarlet-Ef1a-rtTA-BFP
pEfla-rtTA-Antares1
pCMVR8.74
pMD2.G

Sources of genetic elements: Anabaena flos. aquae GV gene cluster: M. G. Shapiro; NFATx4: pSIRV-NFAT-GFP (Addgene #118031); Antares: pNCS-Antares (Addgene #74279); Lentivirus packaging plasmids: pCMVR8.74 (Addgene #22036), pMD2.G (Addgene #12259).

Lentivirus Packaging DMEM+media for packaging was prepared by supplementing DMEM with 4.5 g/L glucose, 2 mM glutamine, 1 mM pyruvate, 20 mM HEPES, 1×MEM-Non-Essential Amino Acids (0.1 mM), and 10% fetal bovine serum. HEK293T cells (passaged no more than 20 times) were seeded in 10 cm plates and grown in DMEM+until they reached 80-90% confluence. A mixture of 22 μg pLV (plasmid with the gene of interest), 22 μg pCMVR8.74, and 4.5 μg pMD2.G was prepared. These plasmids were combined with PEI Max at a PEI to DNA ratio of 3:1, incubated at room temperature for 12 minutes, and then added to the cells. Cells were incubated at 37° C. and 5% CO2 for 12 hours. After this period, the transfection media was replaced with 10 mL of fresh DMEM+. After 48 hours, the spent media was collected and centrifuged at 500 g for 10 minutes to remove debris. The viral supernatant was then concentrated 100-fold into 1× phosphate-buffered saline (PBS) using LentiX Concentrator (Takara Bio), following the manufacturer's protocol. The concentrated virus was aliquoted and stored at −80° C. for up to 6 months.

Lentivirus Measuring titer For the NFAT-inducible virus, LentiX GoStix Plus (Takara Bio) was used to measure the titer, following manufacturer's instructions. All other viruses were functionally titrated using the following protocol. Cells were seeded in 24-well plates in DMEM and grown to 70-90% confluence. Three wells were trypsinized, and the average of their cell count was used in calculating the titer, described below. A serial dilution of the virus was prepared in fresh DMEM and added to each well. Plates were spun for 50 minutes at 800 g at 35° C. and then incubated at 37° C. and 5% CO2 for 16-20 hours. The media was then aspirated and replaced with fresh DMEM, supplemented with doxycycline (1 μg/mL) for the HEK-TetON and HEK-TRE cell lines. Cells were incubated for 24 hours and then prepared for flow cytometry. The functional titer of each virus was calculated using the following formula:

$$TU / mL = \frac{average cell count \times \% fluorescence positive}{viral volume}$$

Only dilutions resulting in less than 30% fluorescent cells were used for the final calculation. This threshold was chosen because it ensures that the probability of any cell receiving more than one functional viral particle is less than 26%.

The following HEK293T-based cell lines were used for functional titering: 1) wild-type HEK293T cells used to titrate any virus expressing a fluorescent protein constitutively, 2) HEK-TetON cells (ATCC) used to titrate any virus expressing a fluorescent protein downstream of the TRE promoter, 3) HEK-TRE cells engineered to express mScarlet-I downstream of TRE, and used to titrate any virus expressing rtTA.

Lentivirus Transduction To transduce HEK293T cells, the cells were seeded in 12-well plates 24 hours before transduction and grown to 70-90% confluence. On the day of transduction, cells from three wells were counted and averaged to determine the viral volume needed for each desired MOI. Lentivirus was mixed with fresh media and added to each well with 10 μg/mL of polybrene (Millipore Sigma). Cell culture plates were spun at 800 g for 50 minutes at 35° C., and incubated at 37° C. for 16-20 hours. The viral media was then removed, and the cells were passaged for downstream applications.

To transduce suspension cells (Jurkats and primary T cells), the cells were counted on the day of transduction and one million cells are mixed with fresh media and virus at the desired MOI. The cells were spun at 1000 g for 50 minutes at 35° C., then incubated at 37° C. for 16-20 hours. Viral media was then removed, and the cells were passaged for further use.

In vitro T cell activity induction For chemical induction, Jurkat cells were stimulated with 50 ng/ml phorbol 12-myristate 13-acetate (PMA) and 1.3 μM ionomycin for 24 hours and immediately used for flow cytometry and ultrasound imaging. For receptor-mediated induction, Jurkat cells were co-cultured with Raji cells at a 1:1 ratio in media supplemented with 1 ng/ml CD19-CD3 BTE (BPS Bioscience, Catalog #100441). Similar to chemical induction experiments, cells were used for flow cytometry and ultrasound imaging after 24 hours of co-culture.

T cell isolation, transduction, and sorting T cells were isolated from frozen human peripheral blood mononuclear cells (STEMCELL) using the EasySep Human T Cell Isolation Kit (STEMCELL), and activated with Dynabeads Human T-Activator CD3/CD28 beads (Gibco). T cells were cultured in RPMI supplemented with GlutaMAX (Gibco) and recombinant human IL-2 at 100 U/mL (Miltenyi Biotec). 16-20 hours after isolation/activation, the cells were transduced with lentivirus as described above. Lentivirus was removed 16-20 hrs after transduction, and the cell density was maintained at 1e6 cells/mL. Activation beads were removed on day 5 post-isolation/activation. Fluorescence was induced with doxycycline for 2 days, and GFP-positive cells were sorted using the MACSQuant Tyto Cell Sorter (Miltenyi Biotec) on day 7. The sorted cells were then expanded in vitro for further use.

Cell preparation and loading for in vitro imaging Adherent cells were seeded 24 hours before doxycycline induction and allowed to reach 70-80% confluence. Suspension cells were resuspended to 0.8e6 cells/mL on the day of induction. Unless stated otherwise, doxycycline (1 μg/mL) was added every 24 hours for 72 hours. Suspension cells were passaged to keep their density below 1e6 cells/mL. On the day of imaging, cells were trypsinized and resuspended. They were briefly mixed with 1% melted agarose in 1×PBS (1:1 dilution at 42° C.) and loaded into agarose phantoms at a final concentration of 25 million cells/mL for all experiments, except detection limit experiments. Primary T cells were loaded at 50 million cells/mL.

In vitro killing assay Primary T cells were mixed with Raji cells to achieve a final density of 1 million cells/mL. To induce receptor-mediated T cell cytotoxicity, CD19-CD3 BTE (1 ng/ml, BPS Bioscience) and IL-2 (100 U/mL) were added. After 24 hours of co-culture, the percentage of live Raji cells was measured using 7-AAD (BioLegend) staining with flow cytometry, following the manufacturer's protocol.

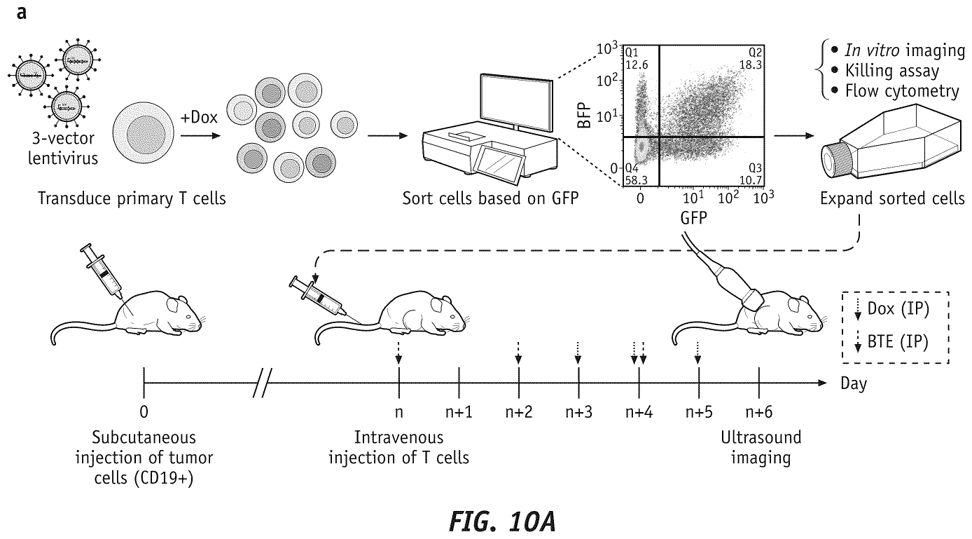

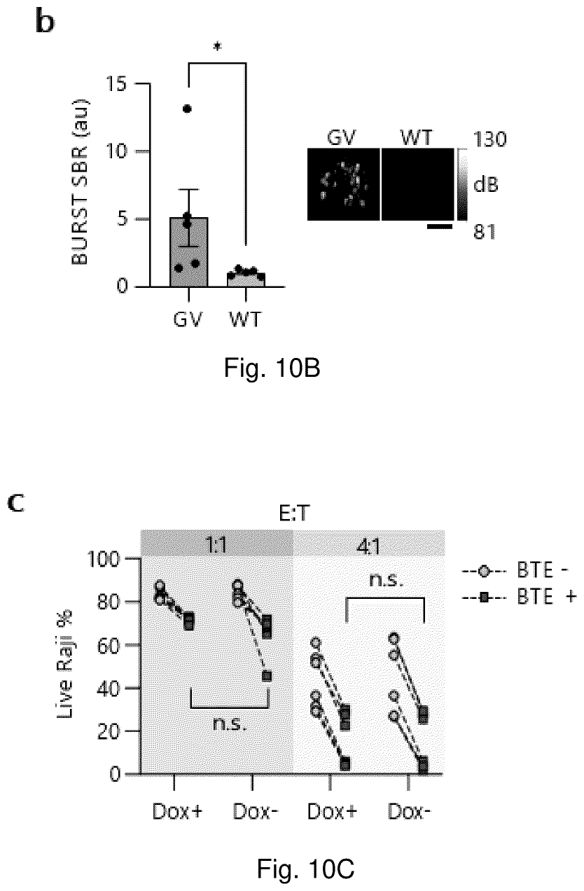

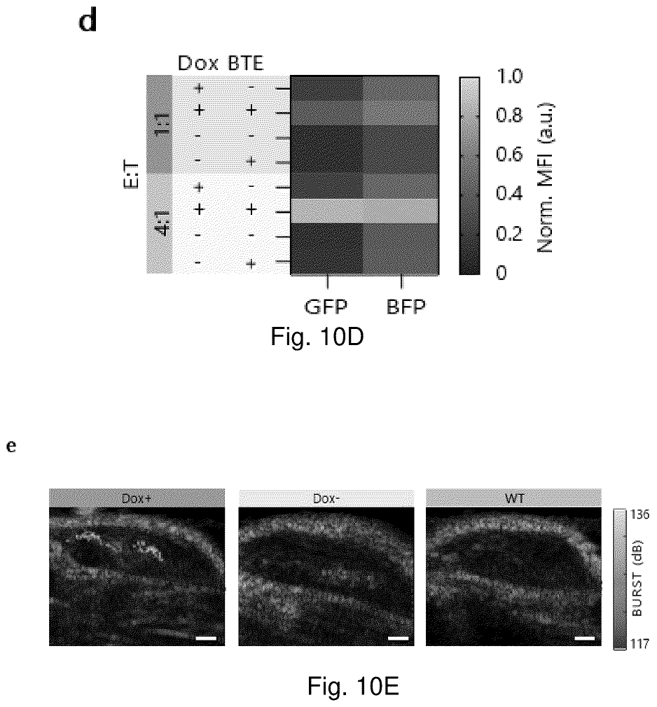

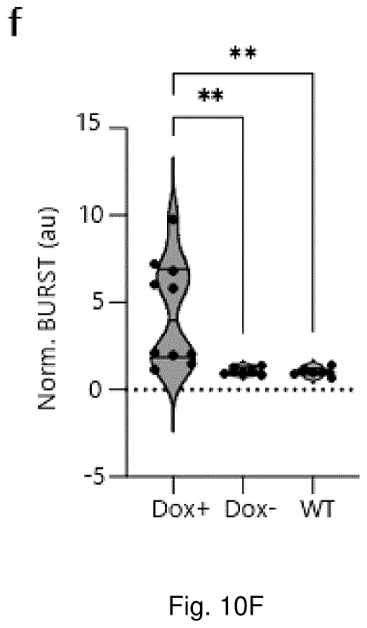

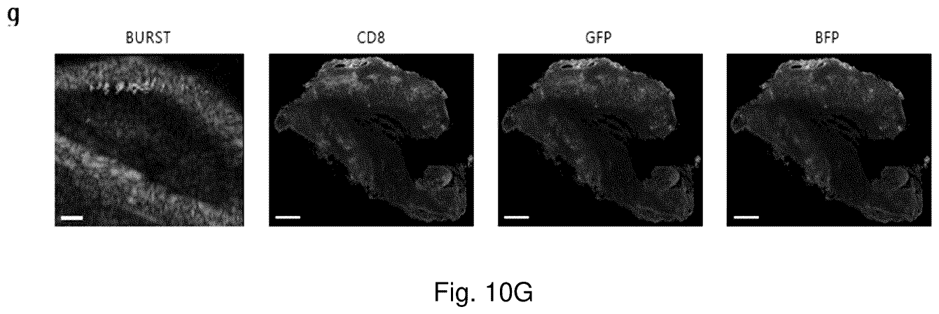

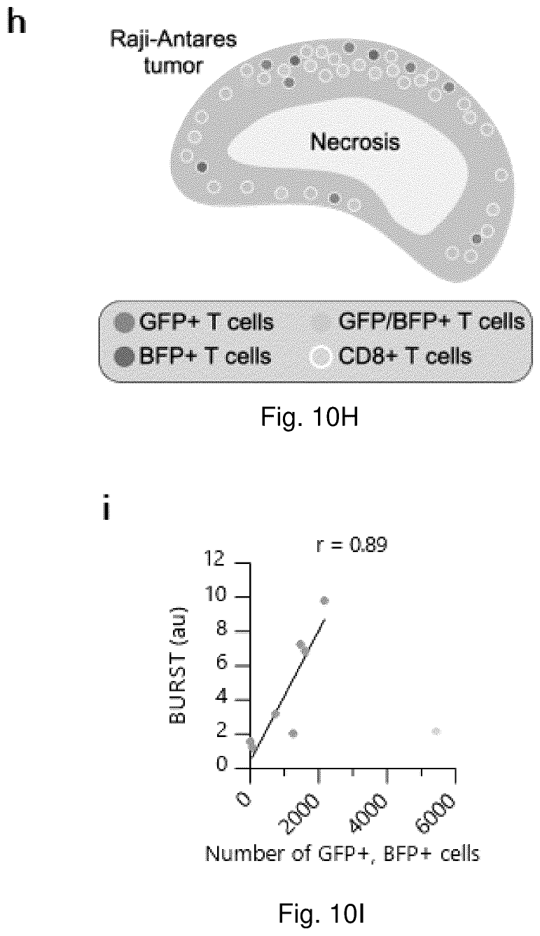

Tumor implantation and T cell injections in mice NSG mice (NOD-scid IL2Rgamma) aged 3 to 8 months were used. 4 million Raji cells expressing the Antares fluorescence reporter under Ef1a promoter were resuspended in matrigel on ice and injected subcutaneously near the flank. When tumors reached a measurable size between days 12 and 17 after implantation, 10 million T cells were injected systemically. BTE (0.25 mg/kg) and doxycycline (4 mg/kg) were administered intraperitoneally according to the timeline in FIG. 10A, with the first BTE dose administered 30 min before T cell injection. Hair around the tumor was shaved immediately before imaging, and mice were anesthetized under 1-2.5% isoflurane during imaging.

Flow cytometry of tumors Tissues were harvested, mechanically dissociated by chopping with a razor blade, and transferred to a digestion buffer (Leibovitz's media with 0.1 mg/mL DNase I and 2 mg/mL Collagenase P). The tissue samples were incubated at 37° C. for 1 hour with continuous rotation. After incubation, samples were washed twice with flow buffer (HBSS with 0.25% BSA) and stained with an Alexa Fluor 647-conjugated anti-human CD8 antibody (BioLegend) according to the manufacturer's protocol. Samples were resuspended in flow buffer, filtered through a 70 μm cell strainer, and analyzed by flow cytometry (MacsQuant Analyzer 10).

Histology of tumors Tumors were extracted from mice after ultrasound imaging and fixed overnight in 4% paraformaldehyde at 4° C. with continuous rotation. They were then immersed in 30% sucrose for 48 hours before embedding in O.C.T. Compound (Fisher Scientific) at −80° C. The frozen tissue was cryosectioned at 20 μm thickness, with 3 to 6 slices collected every 1 mm. The location of each cryosectioned slice was recorded, in order to match to the corresponding BURST plane within 1 mm accuracy. CD8+ T cells were stained overnight at 4° C. using Alexa Fluor 647-conjugated anti-human CD8 antibody (BioLegend). Confocal imaging was performed using an inverted laser scanning confocal microscope. Cell counting was performed using the Imaris software's Spots detection function.

(Ultrasound data acquisition and In vitro imaging: Ultrasound imaging for in vitro experiments was performed using a Verasonics Vantage programmable ultrasound scanning system with an L22-14vX 128-element linear array transducer (Verasonics). Image acquisition was conducted using previously published BURST and xAM scripts. For BURST imaging, the transmit waveform was set to a frequency of 15.6 MHz, with a 67% intra-pulse duty cycle, and 7 half-cycle transmits. The BURST pulse sequence consisted of a single low-pressure frame (transducer voltage: 1.6 V; peak positive pressure: 0.4 MPa) followed by forty-five high-pressure frames (transducer voltage: 15 V; peak positive pressure: 3.6 MPa). The programmable transmit focus was set to 8 mm to align with the fixed elevation focus of the transducer.

All in vitro BURST images were reconstructed using a temporal-template unmixing algorithm across individual pixel locations in the frame stack [44]. Normalized signal-to-background ratio (SBR) was defined as the average BURST signal in an experimental well divided by the average signal in WT/non-transduced control wells for each experiment.

For xAM imaging, cells were imaged over a range of pressures (peak positive pressure 0.2 MPa to 1.1 MPa; transducer voltage 3.5 V to 9.5 V). The pressure that yielded the highest signal-to-noise ratio before GV collapse was selected for quantification. xAM SBR was defined as the average xAM signal in a well divided by the average signal in a ROI outside the well and inside the phantom.

(Ultrasound data acquisition and In vivo imaging: For in vivo experiments, the same Verasonics Vantage system and transducer can be used as the in vitro experiments. The BURST pulse sequence began with a single low-pressure frame (transducer voltage: 1.6 V), followed by five high-pressure frames (transducer voltage: 25 V). Multiple transmit foci were employed to capture the entire tumor, with a BURST image acquired at each focus. These images were summed to create a composite BURST image. Each cross section of tumor was imaged twice, with the second acquisition used for background normalization.

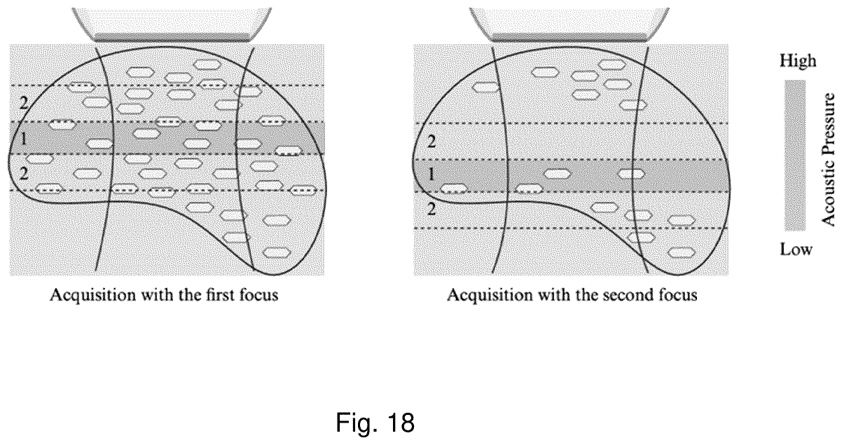

BURST images were reconstructed by subtracting the 5th post-collapse frame from the collapse frame to isolate collapse-based nonlinear scattering from linear scatterers. Images with different transmit foci were then summed to create a composite image, which was subsequently processed with a Gaussian blur filter (σ=0.5). The region of interest (ROI) was defined as the entire region within the tumor, excluding any contrast outside this ROI from analysis. The normalized signal-to-background ratio (SBR) was calculated by dividing the BURST signal within the ROI of the first acquisition by the signal within the same ROI of the second acquisition. The anticipated depth-dependent distribution of captured BURST signal is shown in FIG. 18.

### Example 1: Identification of Gvp Genes and Protein Sequences Through Alignment

Gvp genes and related protein can be identified through alignment of sequences in databases or identified through wet bench experiments with an approach and techniques identifiable by a skilled person.

Taking as gvpA/B as an example, the identification can be performed using consensus sequence: SSSLAEVLDRILDKGXVIDAWARVSLVGIEILTIEARVVIASVDTYLR (SEQ ID NO: 3) wherein X can be any amino acid. LDRILD (SEQ ID NO: 4), RILDKGXVIDAWARVS (SEQ ID NO: 5) wherein X can be any amino acid, and/or DTYLR (SEQ ID NO: 6), and/or of exemplary gvpA and gvpB protein sequences already identified, as it will be understood by a skilled person.

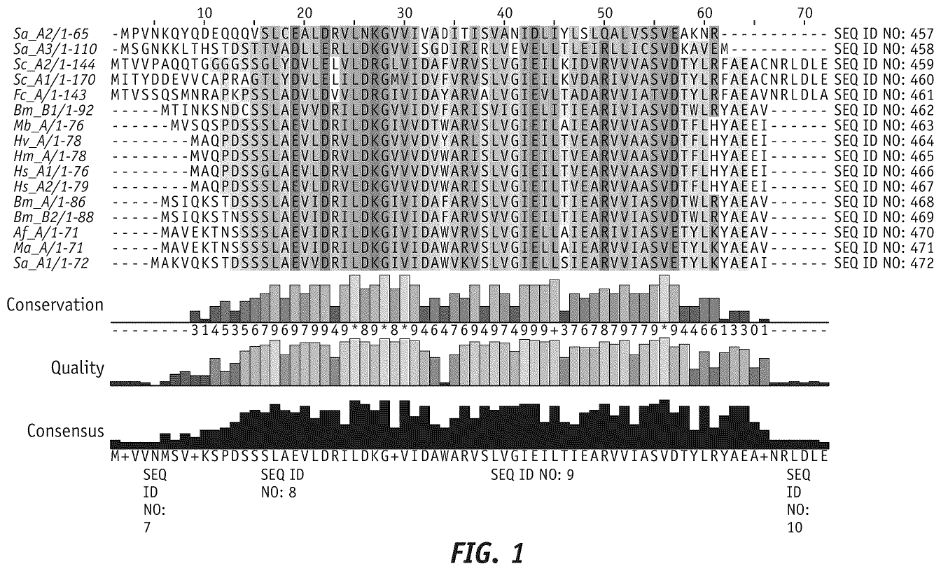

FIG. 1 shows an exemplary Clustal omega alignment of amino acid sequences of selected exemplary gvpA and gvpB proteins.

The gvpA and gvpB proteins shown are from the following species: Sa_A2, Serratia sp. ATCC 39006 gvpA2; Sa_A3, Serratia sp. ATCC 39006 gvpA3; Sc_A2, Streptomyces coelicolor gvpA2; Sc_A1, Streptomyces coelicolor gvpA1; Fc_A, Frankia sp. gvpA; Bm_B1, B. megaterium gvpB1; Mb_A, Methanosarcina barkeri gvpA; Hv_A, Halorubrum vacuolatum gvpA; Hm_A, Haloferax mediterranei gvpA; Hs_A1, Halobacterium sp. NRC-1 gvpA1; Hs_A2, Halobacterium sp. NRC-1 gvpA2; Bm_A, B. megaterium gvpA; Bm_B2, B. megaterium gvpB2; Af_A, A. flos-aquae gvpA; Ma_A; Sa_A1, Serratia sp. ATCC 39006 gvpA1.

The bottom row of FIG. 1 indicated as “Consensus” shows an exemplary consensus sequence derived from alignment of the gvpA and gvpB amino acid sequences shown.

Homology-based searching (e.g., BLAST alignment) of sequences of proteins encoded in the genome of a prokaryotic organism compared to the exemplary consensus sequence shown in FIG. 1 can be used to identify gvpA and/or gvpB protein sequences in the prokaryotic organism.

### Example 2: Identification Gvp Genes and Protein Sequences Through Phylogenesis

Gvp genes and related protein can be identified based on phylogenetic relationships of sequences in databases or identified through wet bench experiments with an approach and techniques identifiable by a skilled person.

In particular, exemplary gvpA, gvpF and gvpN genes and proteins were identified phylogenetic relationships as shown below.

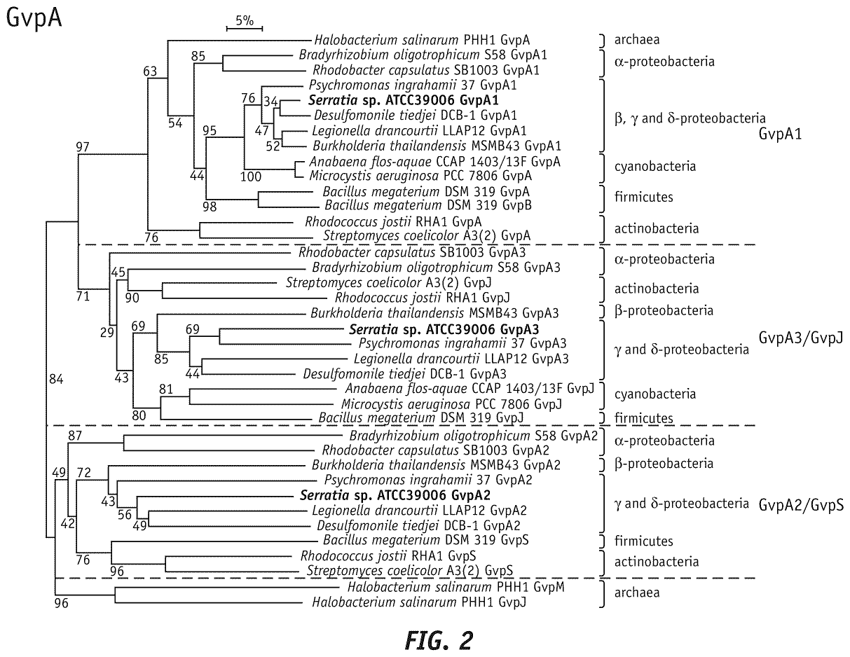

FIG. 2 shows exemplary phylogenetic relationships of the gvpA protein sequences from the indicated prokaryotic species [1]. Table 6 lists examples of GV protein sequences from a number of prokaryotic species.

Identification of a gvpA/B protein can be performed by comparing the sequence of an unknown protein in a prokaryotic cell with that of a known gvpA sequence from the closest phylogenetic relative of the prokaryotic species, such as those indicated in the exemplary phylogenetic tree diagram in FIG. 2. Alternatively, identification of gvpA/B can be done through protein alignment algorithms (e.g. BLAST) with the gvpA/B consensus sequence provided in this document, where the protein identity has 60% or higher to this sequence.

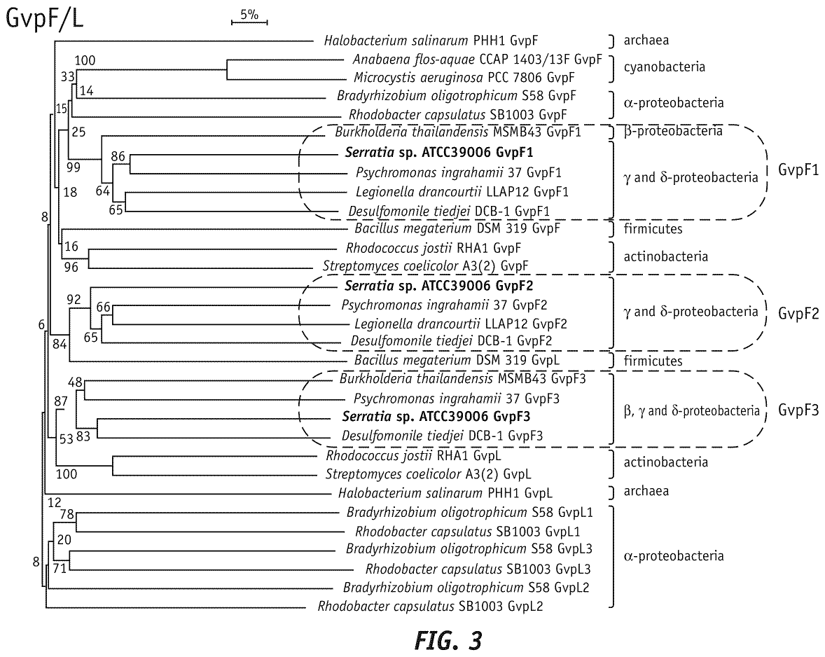

FIG. 3 shows exemplary phylogenetic relationships of the gvpF and gvpL protein sequences from the indicated prokaryotic species [1]. In some embodiments described herein, the identification of a gvpF protein can be performed by comparing the sequence of an unknown protein in a prokaryotic cell with that of a known gvpF sequence from the closest phylogenetic relative of the prokaryotic species, such as those indicated in the exemplary phylogenetic tree diagram in FIG. 3.

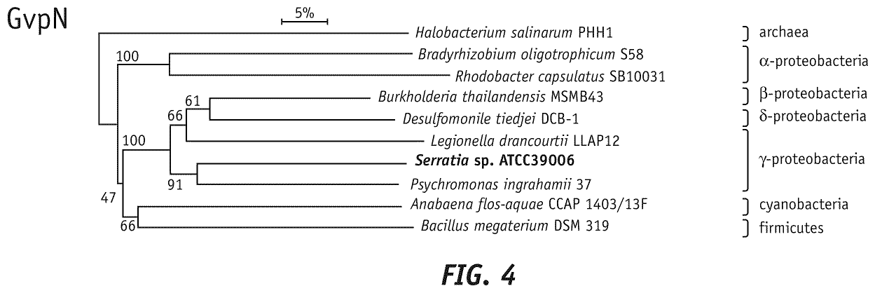

FIG. 4 shows exemplary phylogenetic relationships of the gvpN protein sequences from the indicated prokaryotic species [1]. In some embodiments described herein, the identification of a gvpN protein can be performed by comparing the sequence of an unknown protein in a prokaryotic cell with that of a known gvpN sequence from the closest phylogenetic relative of the prokaryotic species, such as those indicated in the exemplary phylogenetic tree diagram in FIG. 4.

The protein sequences provided in Table 6 can also be used with protein alignment algorithms to identify gvps. Where the using BLAST or other tools, if the top 100 based on protein identity or 100 lowest E-values are identified as “gas vesicle protein” or “gvp” or “gas vesicle structural protein”, the protein can be designated as a gas vesicle protein.

### Example 3: Identification of Gvp Genes and Proteins Through Analysis of Configuration Vesicle Gene Clusters in Prokaryotes

Identification of gvp genes and proteins can be performed also GV cluster configuration of gas vesicle gene clusters in prokaryotes which can be used to identify the specific genes forming a GV cluster in a microorganism, in combination with use of consensus sequences, alignment and/or phylogenetic analysis of GV clusters.

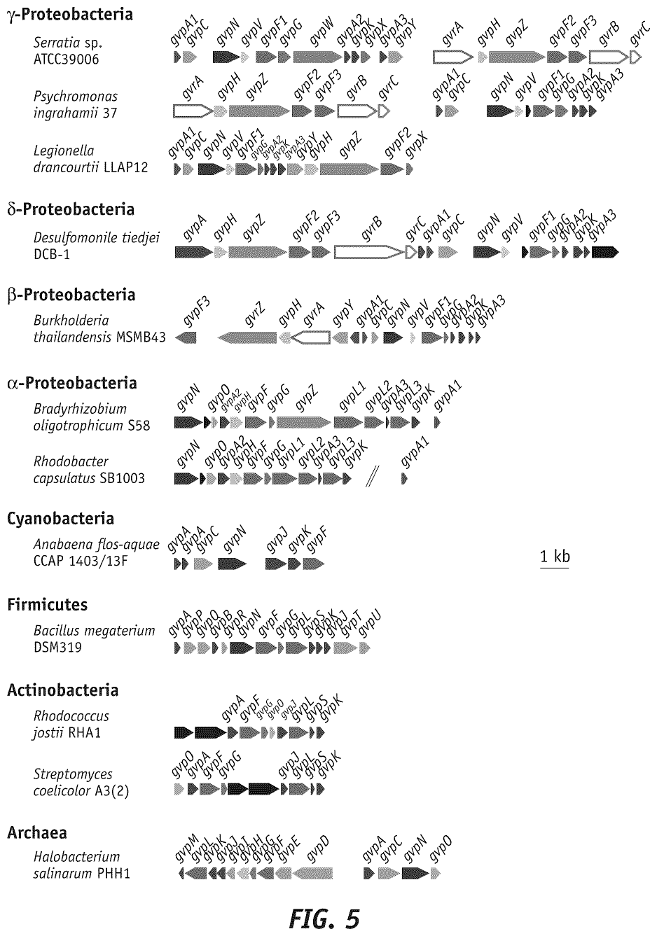

FIG. 5 shows diagrams illustrating the organization of exemplary gas vesicle gene clusters. Gas vesicle gene clusters from the indicated organisms are shown, with genes shown as block-shaped arrows, and genes of predicted similar function indicated in the same shade of grey. The direction of the transcription of genes within a gene cluster is indicated by the direction of the block-shaped arrows, and genes grouped together having block arrows pointed in the same direction are typically organized in the same operon. The scale bar indicates 1 kb [1].

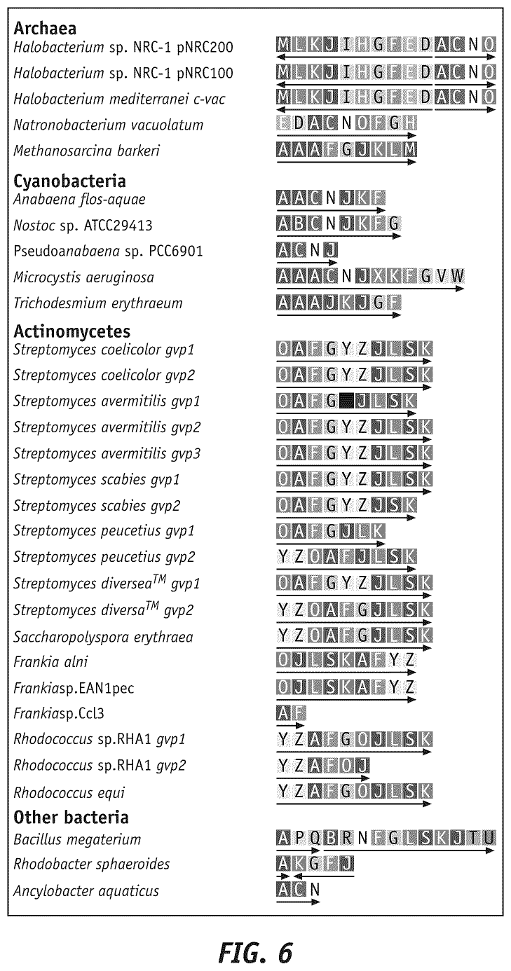

In addition, FIG. 6 shows diagrams illustrating organization of exemplary gvp gene clusters, wherein each letter indicates a gvp gene, and an arrow beneath a group of letters indicates an operon, with the direction of the arrow indicating the direction of transcription [2].

To identify gvp genes and gvp gene cluster, the following methodology can be used:
1. Using the 60+% gvpA/B and/or 50%+gvpN consensus sequences and/or gvp sequences provided in Table 6, identify gvp genes on the genome of the prokaryote.
2. For a gvp gene identified, test the next 10 protein coding sequences on both side of the gene to determine if it is gvp gene. Using BLAST or other tools, if the top 100 based on protein identity or 100 lowest E-values are identified as “gas vesicle protein” or “gvp” or “gas vesicle structural protein”, the protein can be designated as a gas vesicle protein.
3. If the adjacent genes are labeled as gvp gene, continue testing the next 10 protein coding sequences on both sides of the protein, moving away from the labeled gvp genes. Use criterion 2 to continue identifying gvp genes. If the adjacent 10 genes are not marked as gvp genes continue to next part.
4. The genes at the extreme ends will mark the edge of the gene cluster and all the genes inside are part of the gene cluster than can be tested for heterologous expression gas vesicle in bacteria/mammalian cells. In some cases, there can be one or more gene clusters encoding gvp genes, therefore all the gene clusters are tested during heterologous expression.

In particular, the above methodology can be one way to identify gvp gene clusters in an unannotated or mis-annotated genome as will be understood by a skilled person.

### Example 4: Amino Acid Sequences of Exemplary GV Proteins Including GVS and GVA Proteins

Several gvp genes and related proteins have been identified and are available in accessible databases.

In particular, Tables 6-10 show amino acid sequences of exemplary GVS (gvpA/B or gvpC) and GVA proteins from several exemplary prokaryotic species. In particular, these exemplary amino acid sequences can be used as reference amino acid sequences in some embodiments for homology-based searches for related GVS and GVA proteins.

TABLE 6
Amino acid sequences of exemplary_gvpA/B,_gvpF,_gvpF/L,_gvpG,_gvpJ,_gvpK,_gvpL,
gvpN,_gvpV,_gvpW,_gvpR,_gvpS,_gvpT, and_gvpU proteins

| | | SEQ ID |
|---|---|---|
| Species, protein; | Amino acid sequence | NO.: |
| gvpA/B | | |
| Ana-family- | MAVEKTNSSSSLAEVIDRILDKGIVIDAWVRVSLVGIELLAIEARXV | 33 |
| consensus_gvpA | IASVETYLKYAEAVGLTXSAAVPAX | |
| Aphanizomenon-flos- | MAVEKTNSSSSLAEVIDRILDKGIVIDAWVRVSLVGIELLAIEARIVI | 34 |
| aquae_gvpA | ASVETYLKYAEAVGLTQSAAVPA* | |
| Aphanothece- | MAVEKTNSSSSLGEVVDRILDKGVVVDLWVRVSLVGIELLAVEAR | 35 |
| halophytica_gvpA | VVVASVETYLKYAEAVGLTSSAAVPAE* | |
| Anabaena-flos- | MAVEKTNSSSSLAEVIDRILDKGIVIDAWVRVSLVGIELLAIEARIVI | 36 |
| aquae_gvpA | ASVETYLKYAEAVGLTQSAAVPA* | |
| Ancylobacter- | MAVEKINASSSLAEVVDRILDKGVVVDAWVRVSLVGIELLAVEAR | 37 |
| aquaticus_gvpA | VVVAGVDTYLKYAEAVGLTASAQAA* | |
| Aquabacter- | MAVEKINASSSLAEVVDRILDKGVVVDAWVRVSLVGIELLAVEAR | 38 |
| spiritensis_gvpA | VVVAGVDTYLKYAEAVGLTAGAQAA* | |
| Arthrospira-sp-PCC- | MAVEKVNSSSSLAEVIDRILDKGIVIDAWVRVSLVGIELLSVEARV | 39 |
| 8005_gvpA | VIASVETYLKYAEAVGLTAQAAVPSV* | |
| Calothrix-sp-strain- | MAVEKTNSSSSLAEVIDRILDKGIVVDAWVRVSLVGIELLAIEARIV | 40 |
| PCC-7601_gvpA | IASVETYLKYAEAVGLTQSAAVPA* | |
| Dactylococcopsis- | MAVEKTNSSSSLGEVVDRILDKGVVVDLWVRVSLVGIELLAVEAR | 41 |
| salina-PCC- | VVIASVETYLKYAEAVGLTSSAAVPAE* | |
| 8305_gvpA1 | | |
| Dolichospermum- | MAVEKTNSSSSLAEVIDRILDKGIVIDAWVRVSLVGIELLAIEARIVI | 42 |
| circinale- | ASVETYLKYAEAVGLTQSAAVPA* | |
| AWQC131C_gvpA | | |
| Dolichospermum- | MAVEKTNSSSSLAEVIDRILDKGIVIDAWVRVSLVGIELLAIEARIVI | 43 |
| lemmermannii_gvpA | ASVETYLKYAEAVGLTQSAAVPA | |
| Enhydrobacter- | MAVEKMNASSSLAEVVDRILDKGIVIDAWVRVSLVGIELLAVEAR | 44 |
| aerosaccus_gvpA1 | VVVAGVDTYLKYAEAVGLTAGAEAA* | |
| Lyngbya- | MAVEKVNSSSSLAEVVDRILDKGIVVDAWVRVSLVGIELLAIEAR | 45 |
| confervoides- | VVIASVETYLKYAEAVGLTAQAAVPAS* | |
| BDU141951_gvpA | | |
| Nostoc-punctiforme- | MAVEKVNSSSSLAEVIDRILDKGIVIDAWVRVSLVGIELLSIEARIVI | 46 |
| PCC-73102_gvpA | ASVETYLRYAEAVGLTSQAAVPSAA* | |
| Nostoc-sp-PCC- | MAVEKTNSSSSLAEVIDRILDKGIVVDAWVRVSLVGIELLAIEARIV | 47 |
| 7120_gvpA | IASVETYLKYAEAVGLTQSAAMPA* | |
| Microchaete- | MAVEKTNSSSSLAEVIDRILDKGIVVDAWVRVSLVGIELLAIEARIV | 48 |
| diplosiphon_gvpA | IASVETYLKYAEAVGLTQSAAVPA* | |
| Microcystis- | MAVEKTNSSSSLAEVIDRILDKGIVIDAWARVSLVGIELLAIEARVV | 49 |
| aeruginosa-NIES- | IASVETYLKYAEAVGLTQSAAVPA* | |
| 843_gvpA1 | | |
| Microcystis- | MAVEKTNSSSSLAEVIDRILDKGIVIDAWARVSLVGIELLAIEARVV | 50 |
| aeruginosa-NIES- | IASVETYLKYAEAVGLTQSAAVPA* | |
| 843_gvpA2 | | |
| Microcystis- | MAVEKTNSSSSLAEVIDRILDKGIVIDAWARVSLVGIELLAIEARVV | 51 |
| aeruginosa-NIES- | IASVETYLKYAEAVGLTQSAAVPA* | |
| 843_gvpA3 | | |
| Microcystis-flos- | MAVEKTNSSSSLAEVIDRILDKGIVIDAWARVSLVGIELLAIEARVV | 52 |
| aquae-TF09_gvpA | IASVETYLKYAEAVGLTQSAAVPA* | |
| Phormidium-tenue- | MAVEKVNSSSSLAEVVDRILDKGIVIDAWVRVSLVGIELLAIEARV | 53 |
| NIES-30_gvpA | VIASVDTYLKYAEAVGLTAQAAVPAA* | |
| Planktothrix- | MAVEKVNSSSSLAEVIDRILDKGIVIDAWVRVSLVGIELLSIEARIVI | 54 |
| agardhii_gvpA | ASVETYLKYAEAVGLTAQAAVPSV | |
| Planktothrix- | MAVEKVNSSSSLAEVIDRILDKGIVIDAWVRVSLVGIELLSIEARIVI | 55 |
| rubescens_gvpA | ASVETYLKYAEAVGLTAQAAVPSV* | |
| Pseudanabaena- | MAVEKVNSSSSLAEVIDRILDKGIVIDAWVRVSLVGIELLSIEARVV | 56 |
| galeata-PCC- | IASVETYLKYAEAVGLTASAAVPAA | |
| 6901_gvpA | | |
| Stella-vacuolata_ | MAVEKINASSSLAEVVDRILDKGVVVDAWVRVSLVGIELLAVEAR | 57 |
| gvpA | VVVAGVDTYLKYAEAVGLTAGAQTA* | |
| Trichodesmium- | MAVEKVNSSSSLAEVIDRILDKGVVVDAWIRLSLVGIELLTIEARIV | 58 |
| erythraeum- | VASVETYLKYAEAVGLTTLAAAPGEAAA* | |
| IMS101_gvpA3 | | |
| Trichodesmium- | MAVEKVNSSSSLAEVIDRILDKGVVVDAWVRLSLVGIELLTIEARI | 59 |
| erythraeum- | VIASVETYLKYAEAVGLTTLAAEPAA* | |
| IMS101_gvpA4 | | |
| Tolypothrix-sp.-PCC- | MAVEKTNSSSSLAEVIDRILDKGIVVDAWVRVSLVGIELLAIEARIV | 60 |
| 7601_gvpA1 | IASVETYLKYAEAVGLTQSAAVPA* | |
| Tolypothrix-sp.-PCC- | MAVEKTNSSSSLAEVIDRILDKGIVVDAWVRVSLVGIELLAIEARIV | 61 |
| 7601_gvpA2 | IASVETYLKYAEAVGLTQSAAVPA* | |
| Halo-family- | MAQPDSSSLAEVLDRVLDKGVVVDVWARXSLVGIEILTVEARVV | 62 |
| consensus_gvpA | AASVDTFLHYAEEIAKIEQAELTAGAEA-XPAPEA | |
| Halobacterium- | MAQPDSSGLAEVLDRVLDKGVVVDVWARVSLVGIEILTVEARVV | 63 |
| salinarum_gvpA1 | AASVDTFLHYAEEIAKIEQAELTAGAEAAPEA | |
| Halobacterium- | MAQPDSSSLAEVLDRVLDKGVVVDVWARISLVGIEILTVEARVVA | 64 |
| salinarum_gvpA2 | ASVDTFLHYAEEIAKIEQAELTAGAEAPEPAPEA | |
| Halobacterium- | MAQPDSSGLAEVLDRVLDKGVVVDVWARVSLVGIEILTVEARVV | 65 |
| salinarum-NRC- | AASVDTFLHYAEEIAKIEQAELTAGAEAAPEA* | |
| 1_gvpA1 | | |
| Halobacterium- | MAQPDSSSLAEVLDRVLDKGVVVDVWARISLVGIEILTVEARVVA | 66 |
| salinarum-NRC- | ASVDTFLHYAEEIAKIEQAELTAGAEAPEPAPEA* | |
| 1_gvpA2 | | |
| Haloferax- | MVQPDSSSLAEVLDRVLDKGVVVDVWARISLVGIEILTVEARVVA | 67 |
| mediterranei-ATCC- | ASVDTFLHYAEEIAKIEQAELTAGAEAAPTPEA* | |
| 33500_gvpA | | |
| Halogeometricum- | MAQPDSSSLAEVLDRVLDKGVVVDVWARVSLVGIEILTVEARVV | 68 |
| borinquense-DSM- | AASVDTFLHYAEEIAKIEQAELTATAEAAPTPEA* | |
| 11551_gvpA | | |
| Halopenitus-persicus- | MAQPDSSGLAEVLDRVLDKGVVVDVWARVSLVGIEILTVEARVV | 69 |
| strain-DC30_gvpA | AASVDTFLHYAEEIAKIEQAELTAGAEAAPEA | |
| Haloquadratum- | MAQPDSSSLAEVLDRVLDKGIVVDTFARISLVGIEILTVEARVVVA | 70 |
| walsbyi-C23_gvpA | SVDTFLHYAEEIAKIEQAELTAGAEA* | |
| Halorubrum- | MAQPDSSSLAEVLDRVLDKGVVVDVYARLSLVGIEILTVEARVVA | 71 |
| vacuolatum-strain- | ASVDTFLHYAEEIAKIEQAELTAGAEAAPTPEA* | |
| DSM-8800_gvpA | | |
| Halopiger- | MAQPQRRPDSSSLAEVLDRILDKGVVIDVWARISVVGIELLTIEAR | 72 |
| xanaduensis_gvpA1 | VVVASVDTFLHYAEEIAKIEQATAEGDLEELEELEVEPRPESSPQSA | |
| | AE* | |
| Natrialba-magadii- | MAQPQRRPDSSSLAEVLDRVLDKGVVIDIWARVSVVGIELLTVEA | 73 |
| ATCC-43099_gvpA | RVVVASVDTFLHYAEEIAKIEQATAEGDLEDLEELEVEPRPESSPKS | |
| | ATE* | |
| Natrinema- | MAQPQRRPDSSSLAEVLDRVLDKGVVIDVWARISVVGIELLTIEAR | 74 |
| pellirubrum-DSM- | VVVASVDTFLHYAEEIAKIEQATAEGDLDELEELEVEPRPESSPKS | |
| 15624_gvpA1 | AE* | |
| Natronobacterium- | MAQPQRRPDSSSLAEVLDRILDKGVVIDVWARVSVVGIELLTIEAR | 75 |
| gregoryi-SP2_gvpA1 | VVVASVDTFLHYAEEIAKIEQATAEGDLEDLEELEVEPRPESSPQS | |
| | ATE* | |
| Methanosaeta- | MVTSTPDSSSLAEVLDRILDKGIVVDVWARVSLVGIEILTVEARVV | 76 |
| thermophila_gvpA1 | VASVDTFLHYSEEMAKIEQAAIAAAPSA* | |
| Methanosaeta- | MVTSTPDSSSLAEVLDRILDKGIVVDVWARVSLVGIEILTVEARVV | 77 |
| thermophila_gvpA2 | VASVDTFLHYSEEMAKIEQAAIAAAPGVPA* | |
| Methanosarcina- | MVSQSPDSSSLAEVLDRILDKGIVVDVWARVSLVGIEILAIEARVV | 78 |
| barkeri-3_gvpA1 | VASVDTFLHYAEEITKIEIAAKEEKPAIAA* | |
| Methanosarcina- | MVSQSPDSSSLAEVLDRILDKGIVVDTWARVSLVGIEILAIEARVV | 79 |
| vacuolata_gvpA1 | VASVDTFLHYAEEITKIEIAAREEKPVIAA* | |
| Methanosarcina- | MVSQSPDCSSLAEVLDRILDKGIVVDTWARVSLVGIEILAIEARVV | 80 |
| vacuolata_gvpA2 | VASVDTFLHYAEEITKIEIAAREEKPVIAA* | |
| Haladaptatus- | MVQAEPNSSSLADVLDRILDKGVVIDVWARISVVGIEVLTVEARV | 81 |
| paucihalophilus- | VVASVDTFLHYAKEMAKLERASSEDEIDFEQVEVASPEASTS* | |
| DX253_gvpA | | |
| Mega-family- | MSIQKSTXSSSLAEVIDRILDKGIVIDAFARVSXVGIEILTIEARVVIA | 82 |
| consensus_gvpA | SVDTWLRYAEAVGLL-D-VEE-GLP-RX- | |
| Bacillus- | MSIQKSTDSSSLAEVIDRILDKGIVIDAFARVSLVGIEILTIEARVVIA | 83 |
| megaterium_gvpA | SVDTWLRYAEAVGLLTDKVEEEGLPGRTEERGAGLSF* | |
| Bacillus- | MSIQKSTNSSSLAEVIDRILDKGIVIDAFARVSVVGIEILTIEARVVIA | 84 |
| megaterium_gvpB | SVDTWLRYAEAVGLLRDDVEENGLPERSNSSEGQPRFSI* | |
| Serratia-family- | MAKVQKSTDSSSLAEVVDRILDKGIVIDAWXKVSLVGIELLSIEAR | 85 |
| consensus | VVIASVETYLKYAEAIGLTAXAAAPA* | |
| Burkholderia-sp- | MAKVQKSTDSSSLAEVVDRILDKGIVIDVWAKVSLVGIELLSIEAR | 86 |
| Bp5365_gvpA1 | VVIASVETYLKYAEAIGLTATAAAPTA* | |
| Desulfobacterium- | MAKVQKTTDSSSLAEVVDRILDKGIVVDAWAKISLVGIELISIEAR | 87 |
| vacuolatum-DSM- | VVIASVETYLKYAEAIGLTAAAAAPA* | |
| 3385_gvpA | | |
| Desulfomonile-tiedjei- | MAKIAKSTDSSSLAEVVDRILDKGIVIDAWAKVSLVGIELLSVEAR | 88 |
| DSM-6799_gvpA1 | VVIASVETYLKYAEAIGLTASAAAPA* | |
| Isosphaera-pallida- | MAKVTKSTDSSSLAEVVDRILDKGIVIDAFAKVSLVGIELLSVEAR | 89 |
| ATCC-43644_gvpA1 | VVIASVETYLKYAEAIGLTASAATPA* | |
| Lamprocystis- | MAKVANSTDSSSLAEVVDRILDKGIVIDAWIKVSLVGIELLAIEARI | 90 |
| purpurea-DSM- | VIASVETYLKYAEAIGLTAPAAAPA* | |
| 4197_gvpA1 | | |
| Lamprocystis- | MAKVANSTDSSSLAEVVDRILDKGIVIDAWLKVSLVGIELLAVEA | 91 |
| purpurea-DSM- | RVVIASVETYLKYAEAIGLTAPAAAPA* | |
| 4197_gvpA2 | | |
| Legionella-drancourtii- | MAKVQKSTDSSSLAEVIDRILDKGIVIDVWAKVSLVGIELLSIEARV | 92 |
| LLAP12_gvpA1 | VIASVETYLKYAEAIGLTATASHPA* | |
| Psychromonas- | MANVQKTTDSSGLAEVIDRILDKGIVIDAFVKVSLVGIELLSIEARV | 93 |
| Ingrahamii_gvpA1 | VIASVETYLKYAEAIGLTASAATPA* | |
| Psychromonas- | MANVQKSTDSSGLAEVVDRILEKGIVIDAFVKVSLVGIELLSIEARV | 94 |
| Ingrahamii_gvpA4 | VIASVETYLKYAEAIGLTASAATPA* | |
| Serratia-39006_gvpA1 | MAKVQKSTDSSSLAEVVDRILDKGIVIDAWVKVSLVGIELLSIEAR | 95 |
| | VVIASVETYLKYAEAIGLTASAATPA* | |
| Thiocapsa-rosea- | MAKVANSTDSSSLAEVVDRILDKGIVIDAWVKVSLVGIELLAIEAR | 96 |
| strain-DSM-235- | VVIASVETYLKYAEAIGLTAPAAAPA* | |
| Ga0242571-11_gvpA1 | | |
| Other_gvpAs | | |
| Bradyrhizobium- | MAIEKATASSSLAEVIDRILDKGVVIDAFVRVSLVGIELLSIELRAV | 97 |
| oligotrophicum- | VASVETWLKYAEAIGLVAQPMPA* | |
| S58_gvpA1 | | |
| Desulfotomaculum- | MAVKHSVASSSLVEVIDRILEKGIVIDAWARVSLVGIELLAIEARV | 98 |
| acetoxidans-DSM- | VVASVDTFLKYAEAIGLTKFAAVPA* | |
| 771_gvpA1 | | |
| Octadecabacter- | MAVNKMNSSSSLAEVVDRILDKGVVIDAWVRVSLVGIELIAVEAR | 99 |
| antarcticus- | VVIAGVDTYLKYAEAVGLTAEA* | |
| 307_gvpA1 | | |
| Octadecabacter- | MAVSKMNSSSSLAEVVDRILDKGVVIDAWVRVSLVGIELIAVEAR | 100 |
| arcticus-238_gvpA1 | VVIAGVDTYLKYAEAVGLTAEA* | |
| Pelodictyon-luteolum- | MAVEKTIGSSSLVEVIDRILDKGVVVDAWVRMSLVGIELLAIEARV | 101 |
| DSM-273_gvpA1 | VVASVETYLKYAEAIGLTAKAA* | |
| Pelodictyon-luteolum- | MAVEKTIGSSSLVEVIDRILDKGVVVDAWVRVSLVGIELLAIEARV | 102 |
| DSM-273_gvpA2 | VVASVETYLKYAEAIGLTAKAA* | |
| Pelodictyon- | MSVEKTIGSSSLVEVIDRILDKGVVVDAWVRVSLVGIELLAIEARV | 103 |
| phaeoclathratiforme_ | VVASVETYLKYAEAIGLTAKAA* | |
| gvpA1 | | |
| Rhodobacter- | MAIEKSLASASIAEVIDRVLDKGIVVDAFVRISLVGIELLAIELRAV | 104 |
| capsulatus-SB- | VASVETWLKYAEAIGLTVDPQTP* | |
| 1003_gvpA1 | | |
| Rhodobacter- | MAIEKSVASASIAEVIDRILDKGVVIDAFVRVSLVGIELIAIEVRAVV | 105 |
| sphaeroides_gvpA1 | ASIETWLKYAEAVGLTVDPATT* | |
| gvpF | | |
| Anabaena-flos- | MSIPLYLYGIFPNTIPETLELEGLDKQPVHSQVVDEFCFLYSEARQE | 106 |
| aquae_gvpF | KYLASRRNLLTHEKVLEQTMHAGFRVLLPLRFGLVVKDWETIMS | |
| | QLINPHKDQLNQLFQKLAGKREVSIKIFWDAKAELQTMMESHQDL | |
| | KQQRDNMEGKKLSMEEVIQIGQLIEINLLARKQAVIEVFSQELNPF | |
| | AQEIVVSDPMTEEMIYNAAFLIPWESESEFSERVEVIDQKFGDRLRI | |
| | RYNNFTAPYTFAQLDS* | |
| Ancylobacter | MSATLSAPGTANVAVEATAAADGKYLYGIIEAPAPATFDVPAIGG | 107 |
| aquaticus strain | RGDVVHTIALGRLAAVVSNSPRIDYDNSRRNMLAHTKVLEAVMA | |
| UV5_gvpF | RHTLLPVCFGTVGSDAEVIIEKILRERRDELAGLLGQMHGRMELGL | |
| | KASWREEIIFEEVLAENPAIRKLRDALVGRSPDQSHYERIQLGERIG | |
| | QALQRKRQDDEERILERVRPFVHKTRLNKLIGDRMVINAAFLVDA | |
| | AVESRLDASIRAMDEEWGGRLAFKYVGPVPPYNFVTITIHW* | |
| Aphanizomenon flos- | MNTGLYLYGIFPDPIPETVDLQGLDKQSVHSQVVDGFSFLYSDAC | 108 |
| aquae NIES-81_gvpF | QEKYLASRRNLLTHEKVLEQAMHEGFHVLLPLRFGLVVKDWETI | |
| | QKQLIEPYKEQLNELFQKLAGQREVSIKILWDSKSELQAMMESNQ | |
| | DLKQQRDNMEGKKLKMEEIIQIGQLIESNLAARKQTVIQEFFNNLH | |
| | PLAKEIIESEPMTEEMIYNAAFLIPWETESVFSERVEAIDRKFGDRL | |
| | RIRYNNFTAPYTFAQLAS* | |
| Aphanothece | MAEGFYLYGIFPPPGPQTIAVQGLDKQPIFSHTVEGFTFLYSEAQQS | 109 |
| halophytica (strain | RYLASRRNLITHTKVLEEAMEQGFRTLLPLQFGLVVPDWESVSQD | |
| PCC 7418)_gvpF | LLQHQSETLQLLFQRLEGKREVSLKIYWETDAELNALLEENPDLK | |
| | ARRDNLEGKNLSMDEVIQIGQALEQAMERRKQEVITRFEDALIPFA | |
| | VETQENDVLTETMIYNTAFLIPWESEPEFGEAVETVDAEFAPRLKI | |
| | RYNNFTPPYNFVELRE* | |
| Aquabacter spiritensis | MMQTDTLAPAETVAEGKYLYCLIDAPAPDTFASPGIGGRGDVVHT | 110 |
| strain DSM | ITVGRLAAVVSDSPRIEYENSRRNMMAHTKVLEEVMARHTMLPV | |
| 9035_gvpF | CFGTVATGPDPISGKILEGRRDELVGLLEQMRGRLELGLKATWRE | |
| | DVIFAEILQENPAIAKLRDSLVGRSPEKSHFERIRLGEMIGQAMERK | |
| | RRDDEERILERVRPFVHKTKLNKPIGDRMILNAAVLVEAAREAGL | |
| | DQAVRQMDAEWGARLSFKYVGPVPPYNFVTITIHW* | |
| Bacillus- | MSETNETGIYIFSAIQTDKDEEFGAVEVEGTKAETFLIRYKDAAMV | 111 |
| megaterium_gvpF | AAEVPMKIYHPNRQNLLMHQNAVAAIMDKNDTVIPISFGNVFKSK | |
| | EDVKVLLENLYPQFEKLFPAIKGKIEVGLKVIGKKEWLEKKVNEN | |
| | PELEKVSASVKGKSEAAGYYERIQLGGMAQKMFTSLQKEVKTDV | |
| | FSPLEEAAEAAKANEPTGETMLLNASFLINREDEAKFDEKVNEAH | |
| | ENWKDKADFHYSGPWPAYNFVNIRLKVEEK* | |
| Bradyrhizobium | MSNQPIYVYGLIRAEDHQPLAVRAVGDSEQPVNIIGSGNVAALVST | 112 |
| oligotrophicum | IDLPEIMPTRRHMLAHTKVLEAAMANGPVLPMRFGIIVPNPATLLR | |
| S58_gvpF | VIGFRHQELRARLDEIDGRIEVALKASWDEQFMWRQLASEHPDLA | |
| | VSGRTMMGRGEQQSYYDRIELGRAIGAALEERRTAARLQLLQTVT | |
| | PFAVQVKELTPVDDAMFAHLALLVEKGAEPSLYQTVEALERSNDS | |
| | GLKFRYVAPIPPYNFVAVTLDWEQHEQAPRR* | |
| Burkholderia | MNSRNGARYLYAVQHARDVPASLPAGIGGAAVRALTDGDVAAIV | 113 |
| thailandensis sp. | SDTGLAKVRPERRHLLAHHTVIQSLAAAGTVLPVAFGTIATSEVAL | |
| Bp5365 strain | RRMLRKHRNALAGELARLVDHVEMSVRLNWDVTDLFRHLIDVRP | |
| MSMB43_gvpF | DLKAARDAMLALGSAVTRDDKIELGSRFERVLNEERARHAALVD | |
| | EALDACCKEIRRDPPRHETEILHLTCLVRHAELGRFESGVAAASRE | |
| | LDDSLVLKYSGPCPPHHFVNLNMSL* | |
| Chlorobium luteolum | MERDGKYIYCIIGADCECDFGPIGIGGRGDLVSTIGFEGISMVVSDH | 114 |
| DSM 273_gvpF1 | PLNRFVVDPDGILAHQRVIEAVMKEHESVIPVRFGTVAATPDEIRN | |
| | LLDRRYGELSELLLRLRNKVEFNVTGRWHDMAAIYKEVERTHPEI | |
| | KEQRARIESMRDGDGEALKQSLILDTGHQIEAALEVMKEEKFDAV | |
| | ASLFRKTAMASKMNRTTSPDMFMNAAFLIDRGREVEFDGIMEILG | |
| | QKDADRCDYRYSGPLAIFNFVDLRILPEKWEL* | |
| Chlorobium luteolum | MAHEAAEQDGLYIYGIINNSGELDFGPIGIGGREERVYAVIHNDIA | 115 |
| DSM 273_gvpF2 | AVVSRTVVKEFEPRRANMIAHQKVLEAVMVSHAVLPVRFSTVSPG | |
| | HDDMKVEKILEEDYLRLKKLLVKMEGKKEMGLKVMANEEKVYE | |
| | SIITGYDNIRYLRDKLINLPPEKTHYQRVKIGELVAAALEKEVGTY | |
| | KDAVLDALSPIAEEVKVNDSYGSMMVLNAAFLIRTAREEEFDRAV | |
| | NALDDRYHDMMTFKYVGTLPPYNFVNISINIKGR* | |
| Chlorobium luteolum | MNQSIYIYGIVNEPALAASFVETDPDIYAVASMGCSAIVENRPAIDL | 116 |
| DSM 273_gvpF3 | GELDRESLARMLLQHQQTLERLMESGMQLIPLKLGTFVSSAADAA | |
| | CIIEDGYNLIERIFRETEDAHELEVVVKWSSFADLLQEVVSEGDVQ | |
| | ELKREVEARQSSSTEDAIAVGRLIKEKIDRRNAALSASVLRQLGER | |
| | ASQSKRHETMDDEMVLNAAFLVNRGDVDAFVATVEALDSQYLN | |
| | ALHFRIVGPLPCYSFYTLEVTALFEEFIAEKRAVLGLDARSCEADV | |
| | KKAYHAKAKVAHPDVHVPAGANNGADFTVLNEAYMTLHDYYS | |
| | ALRNSASSRHGHEGQDSSSVVFSVKILN* | |
| Dactylococcopsis | MTEGFYLYGIFPPPGPKTIETQGLDKQPIFSHTVEGFTFLYSEAQQS | 117 |
| salina PCC 8305_gvpF | RYLASRRNLITHTKVLEEAMENGSRTLLPLQFGLIVPDWETVVQD | |
| | LLQHQAESLHFFLEKLEGKREVSLKIYWETNAELNALLEENPALK | |
| | ARRDNLEGKQLSMDEVIQIGQALEQEMEGRKQDIISRFEEVLIPFAF | |
| | EIKENDVLTETMIYNTAFLINWDAESDFGEQLEAIDAEFSPRLKIRY | |
| | NNFTPPYNFVELRE* | |
| Desulfobacterium | MSKKNLKRNGRYLYAIIEASEEKTFGSIGMDGSDVYLIVEDKTAA | 118 |
| vacuolatum DSM | VVSDVPNKKIRPQRKNIAAHHAVLNKIMEEITPLPMAFGIIADGEQ | |
| 3385_gvpF | AIRKILADNRDVFREQFATVSGKVEMGMRISYDVPNIFEYFISTDSE | |
| | IRAARDQYFGGNREPSQEAKLELGRMFNRQLNANREEYTNQVIEI | |
| | LDDYCDDIKENKCRNEQEVTSLACLINRSDQKRFEEGVFESARHFD | |
| | NNFSFEYNGPWSPHNFVNILIEL* | |
| Desulfomonile tiedjei | MEKATIKTTGSNGRYLYAVVPGSQERVYGCLGINGGNVYTIAAKD | 119 |
| DSM 6799_gvpF | VAAVVSDVPHQKIRPERRHFAAHQAVLKRVMLDGDLLPMSFGIIS | |
| | QGPKAVRAILSRNNKSVQQQLKRISGKAEMGIKVTWDVPNIFEYFI | |
| | DVNRELREARNKLVQPNYLPTQQEKIEIGRMFEEILNLERERHTKQ | |
| | VERVMSKRCSEIKRSKCRTEIEVMNLSCLVDRTLLSDFEAGVLEAA | |
| | SHFDDSFAFDFNGPWAPHNFVDLEIDV* | |
| Desulfotomaculum | MSTGRYVYCVINSIEPLTFMSGPVGNEPEGVFTVHYKELAAVVSQ | 120 |
| acetoxidans DSM | SSEEKYNVCRENTIAHQKVLEEVLVSHPLLPVRFGTVAQNEEIVKK | |
| 771_gvpF1 | FLLQERYAELRSMLHNVTGKVQMGLKVLWTDMKTVYQEIVEENP | |
| | QIKNLKKKLESKPAETIHYEMIDLGQMVNQALLRKKEKQKEMVL | |
| | KPLQKIALETKESFLYGDQMFVNADFLISRSSLDDFNAKVNELGEF | |
| | FNEQALFKYIGPLPPYNFVTLYVNF* | |
| Desulfotomaculum | MVKNHNTDHLKELYIYGLIGGTPFKDELEKISVIQENTPIYGVWHK | 121 |
| acetoxidans DSM | NIGFAVSAAPDYPLKDLSKESIIQLFVDHQQVLECLRQKFSLIPVKL | |
| 771_gvpF2 | GTVLESVTEAAAVLANNEEKFNDLLNYLKDKVELNLSVSWNDLN | |
| | EVVAKIGEEDEVKKLKQSLLAQEQVSQEDLIKIGKIISFQMQQKKQ | |
| | AAREYIISELRNLWEDYFINEVVDENSILNLTLLAITGKVDDVNKKI | |
| | EYLNQIYRDSLDFSLTKSLLPQGFSTVSIKKITMDQLLLAKDILKLP | |
| | DTASLQDINAARRALLHCYHPDKNDHAAVNKVQEINAAYKLLEE | |
| | YCQENSSDFNVDLITDYYIMKVIKADKSNVNSMNME* | |
| Dolichospermum | MNTDLAHKNFGLYLYGIFPDTIPETLEIKGLDGKSVHSQVVDGFTF | 122 |
| circinale_gvpF | LYSQACQEKYLASRRNLLAHERVLEQTMHEGFHVLLPLRFGLVV | |
| | KDWETIMSQLINPHKEQLHKLFEKLAGQREVSIKILWDAKAELQA | |
| | MMESNHDLRQQRDNMEGKKLSMEEVIQIGQLIESNLQARKQAVIE | |
| | VFTRELNPLAQEIVVSEPMTEEMIYNAAFLIPWDSEPLFSERVESID | |
| | QKFGNRLRIRYNNFTAPYTFALLDS* | |
| Enhydrobacter | MNPPEAYIAGRTAAKSVEDRKARPQDLAEGKYVYAIIACDEPREF | 123 |
| aerosaccus strain | KNRGIGERGDKVHTINHRQMAAVVSDSPTIDYERSRRNMMAHTV | |
| ATCC 27094_gvpF | VLEEVMKEFDLLPLRFGTVASSAESVERQLLVPRYGELSAMLEKM | |
| | RGRSEFGLKAFWHEGVAFGEIVRENARVRKLRDALQGRSLEESYY | |
| | QRIQLGEEVEKALTAIRARDEELILSRLRPFMRDIRTNKIISDRMVL | |
| | NAAFLVERGDVPALDEAIRQLDQEFSERLMFKYVGPVPPYNFVNI | |
| | AINWER* | |
| Isosphaera | MRNAPPTRPGSVTPASPGKPVIDGPARYLYAFTHDLPEGPLADLEG | 124 |
| pallida_ATCC- | LPGARVVVVADGRVAAVVSPCPLGKVRPERQRVAGHHHVLKHL | |
| 43644_gvpF | QDTLGKAILPASFGMVADSEEDLRALLRHHSAAIAEGLVRVQGKV | |
| | EMTVKLRWAPDNVAQAVLGRDPELRQLRDQLYSNGQTPTRDQSL | |
| | DLGRRFHHALERQRDHYAAYLRAALSPLLSELVEEDLRDERDLVH | |
| | WACLIENQRRAGFEAALDRLAEELEDDLVLELTGPWPPHHFVDLD | |
| | LDDDHDDDEEE* | |
| Legionella drancourtii | MDSTSKKPAASNLYLYAIASVNENQEPISFHGIEEQPIDLVPYKDIM | 125 |
| LLAP12_gvpF | LVVSNLSKKKVRPERKNVAVHHAVLNHLMKHNTSMLPIRFGMIA | |
| | DNRKEVQRLLTINYDMLHTKLKMMAGRVEMGVSLSWDVPNIFEY | |
| | LLNRHSQLRETRDKLLANPAHEPSRDEKIEIGALFSQILDEEREVYT | |
| | DTILSLLSPVCCDVVKSTYRNDTEIMNIFCLISAARRDEFEEKIIEAS | |
| | TILDDNFVIKYTGPWPPHNFSKLNLSLE* | |
| Lyngbya confervoides | MPQLLYLYGIFPAPGPQDLEVQGLDQQPIHTHIIDEFVFLYSVAQQE | 126 |
| BDU141951_gvpF | RYLASRKNLLGHERVLEAAMKVGYRTLLPLQFGLIIETWDRVIKE | |
| | LITPRGDALKRLFAKLEGRREVSVKLLWGPDAELNQLMEEDAGLR | |
| | AERDRLEGQQLSMDQIVDIGQAIETAMTERKDDVINAFRQRLNAL | |
| | AIEVLENDPLTDAMIYNTAYLIPWEDEVKFSQAIEELDEQFEDRLRI | |
| | RYNNFTAPYNFAQLDQLS* | |
| Microcystis aeruginosa | MTVGLYLYGIFPEPVPDGLVLQGIDNEPVHSEMIEGFSFLYSAAHK | 127 |
| NIES-843_gvpF | EKYLASRRYLICHEKVLETVMEAGFTTLLPLRFGLVIKTWESVTEQ | |
| | LISPYKTQLKELFAKLSGQREVSIKIFWDNQWELQAALESNPKLKQ | |
| | ERDAMMGKNLNMEEIIHIGQLIEATVLQRKQDIIQVFRDQLNHRA | |
| | QEVIESDPMTDDMIYNAAYLIPWEQEPEFSQNVEAIDQQFGDRLRI | |
| | RYNNLTAPYTFAQLV* | |
| Nostoc punctiforme | MSFYIYGILTLPAPQNLNLEGLDRQPVQIKILDDFAVIYSEAQQERY | 128 |
| ATCC 29133_gvpF | LASRRNLLSHEKVLEEIMQAGDRYLLPVQFGLLVSSWETVSQQLIR | |
| | PHQEELTQLLAKLSGCREVSVKVFWDTEAEIQGLLAEHPNLKTER | |
| | DKLVGQPLSMERVIQIGQVIEQGMSDRKQGIIDVFKGTLNSIAIEVV | |
| | ENTPQVDTMIYNSAYLIPWEAESQFSEHVESLDRQFENRLRIRYNN | |
| | FTAPYNFARLRLTTSN* | |
| Nostoc sp. PCC | MSSGLYLYGIFPDPIPETVTLQGLDSQLVYSQIIDGFTFLYSEAKQE | 129 |
| 7120_gvpF | KYLASRRNLISHEKVLEQAMHAGFRTLLPLRFGLVVKNWETVVT | |
| | QLLQPYKAQLRELFQKLAGRREVSVKIFWDSKAELQAMMDSHQD | |
| | LKQKRDQMEGKALSMEEVIHIGQLIESNLLSRKESIIQVFFDELKPL | |
| | ADEVIESDPMTEDMIYNAAFLIPWENESIFSQQVESIDHKFDERLRI | |
| | RYNNFTAPYTFAQIS* | |
| Octadecabacter | MKREVVRMTDENTINSKYLYAIIKCREQREFIARGIGERGDAVHTI | 130 |
| antarcticus 307_gvpF1 | AYKGLAAVVSDSPVMEYDQSRRNMMAHTAVLEELMEEFTLLPVR | |
| | FNTVAPEAGAIEERLLVPRHEEFTQLLGQIDKRVELGIKAFWHDG | |
| | MIFEEVLRENDSIRKMRDALEGKSVDGSYYERIQLGEKIEQAMIKK | |
| | RVEDEEIILSRIRQHVHKSRSNKTIGDRMVLNGAFLVDANKESDFD | |
| | KAVQLLDQDLGNRLMFKYVGPVPPYNFVNIVVNWGVV* | |
| Octadecabacter | MTVVAEENMTGSVGLYVCAIVAEWESNSALIKCANEAQGEIQLIG | 131 |
| antarcticus 307_gvpF2 | QGGITAVVMVPPEDQPVSRDRQELVRQLLVHQQLVERFTEIAPVL | |
| | PVKFGTLAPDRESVELGLERGREKFFTAFGGLSGKTQFEITVTWDV | |
| | ADVFAKIAKLPAVVKLKVDLVATSESDRPINLDRVGRLVKETLDH | |
| | QRAQTGKVLLDALLPLGVDSIVNPILNDSIVLNLALLVDTDQADAL | |
| | DRCLDELDSTFHGALSFRCVGPMPPHSFATVEINYIEPTQVSHACC | |
| | VLELDAAHNFEEIRSAYHRLARQTQQDIAPDVVVDNKSSSVGIAV | |
| | LNDAYKTLLSFVDAGGPVVVSVQRQEDAYATDIPSSGG* | |
| Octadecabacter | MTDEKKVNSKYLYAIIQCREPRELKARGIGERGDVVHTVVHKGLA | 132 |
| arcticus 238_gvpF1 | AVVSDSPVMEYDQSRRNMMAHTAVLEELMEEFTLLPVRFNTVAP | |
| | EAVAIEERLLVPRHDEFTQLLGQIDKRVELGLKAFWHDGMIFGEV | |
| | LRENDSIRKMRDSLKGQSVDGSYYERIQLGEKIEKALTEKRLEDEE | |
| | MILSRIRPHVHKSRSNKTIGDRMVLNGAFLVDAEKESKFDEAVQSL | |
| | DQDLSDRLMFKYVGPVPPYNFVNIVVNWGES* | |
| Octadecabacter | MRAQKVIPAAEENISGNVGLYVCAIVAERVSCSALIQCANDAPGEI | 133 |
| arcticus 238_gvpF2 | QLIGHGDFTAVVMVPEKDQLVSPDRKELMQQLLVHQQLIEKFMEI | |
| | APVLPVKFATLAPNRESVELGLEVGSEKFSAAFNSLSGKVQFEVIV | |
| | TWDVAEVFAEIAKEPAVAKLKVDLAAMPESYGSVSLEQLGKLVK | |
| | ETLELRRAETGKVLLDALVQVGVDNVVNSILDDSIILNLALLVEAK | |
| | RADAFDRCLDELDSTYHGALTFRCVGPLPPHSFATVEITYLEPAKV | |
| | TEACDILELDVARSTEEVRSAYHRLARKSHPDIVPDVAVGETASVS | |
| | MAVLTDAYKTLLSFVGAGGSVVVSVQRQEASYAADIISSAG* | |
| Pelodictyon | MDIETTKEGRYIYGIIRNSEFIDFGQIGIGKRNDRVYGVIYKDICAV | 134 |
| phaeoclathratiforme_ | VSSTPIIQYEARRANMIAHQKVLEEVMKRFNVLPVRFSTISPHDND | |
| gvpF1 | DAIIKILITDYSRFDELLIKMKGKKELGLKVMADETRIYENIIQKYD | |
| | NIRSLRDKLLNQPADKIHYQRVKIGEMVADALKKEIESYKQQILDI | |
| | LSPIAEDIKITDNYGNLMILNAAFLIKEVKESEFDDSVNKLDEKYGN | |
| | IMTFKYVGTLPPYNFVNLSINTKGV* | |
| Pelodictyon | MEKDGKYVYCIIASTYECNFGAIGIGGRGDLVNTIGFQGLSMVVSD | 135 |
| phaeoclathratiforme_ | HPLNHFVLNPDNILAHQRVIEVVMSQFNSVIPVRFGTVAATPDEIR | |
| gvpF2 | NLLDRRYGELSELLERFENKVEYNLKASWRCMIDIYKEIDKEHVE | |
| | LKQLRREIEGLKDEEKRKLLIVEAGHIIENELQKKKEVEAYEIVTYL | |
| | RKTVVAHKHNKTTGEAMFMNTAFLLNKGREVEFDNIMNDLGEQ | |
| | YKDRSDYYYTGPLPIFNFIDLRILPEKWEL* | |
| Pelodictyon | MDRQGIYIYGFIPNHYLTDIKTILIESGIYSIEYGSIAALVSDTMVDDI | 136 |
| phaeoclathratiforme_ | EYLNREDLAYLLVDHQKKIELIMSTGCSTIIPMQLGTIVNSGNDVIK | |
| gvpF3 | IVKNGLRIINKTFDDIADIQEFDLVVMWNNFPDLIKKISDTPQIRIMK | |
| | EEIANKGSYDQADSINIGKIIKKKIDEKNSKVNLDIMNSLSSLCICVK | |
| | KHESMNDEMPLNSAFLIKKDKENSFIEMVNQLDIKYENLLRYKIV | |
| | GPLPCYSFYTLESKLLNKKEIEKAEKILGIDAYKSESDIKKAYRAKA | |
| | AHAHPDKNNTISAIDNDDFIEINKAYQILLEYSSVFKDSPDHKPDEP | |
| | FYLVKIKK* | |
| Phormidium tenue | MADRYYLYGIFPAPGPAELPLMGLDEQVVQAQQLGDFTFLYSLAC | 137 |
| NIES-30_gvpF | QKRYLSSRKNLLGHEKVLEAAMEQGHRTLLPLQFGLIVESWNQV | |
| | QEDLVTPYAEDLTQLFGRLNGCREVSIKVQWEPSTELEMMMAEN | |
| | ADLRAQRDQLEGTQLGMEQVIFIGQQIESALEERKQGIVDQFRQAL | |
| | SPLAKDVLENAPQTDVMIYNAAFLIPWESEAEFSQAVDAIDSTFGD | |
| | RLRIRYNNFTAPYNFAQLN* | |
| Planktothrix agardhii | MGNGLYLYGILPTNRVRPLALHGLDKQPIQTHPVDEFSFLYSETQQ | 138 |
| str. 7805_gvpF | ERYLASRRNLLGHEDVLEKVMQHGYRSVLPLQFGLIVKDWDHVK | |
| | AQLIIPYQDRLKELFHKLEGKREVGVKIFWEETEELDLLMTENQEL | |
| | REKRDSLEGKRLSMDEIIGIGQEIERAMQDRQQGIIDKFQQILNPLA | |
| | QEIVENDNLTSAMIYNAAYLIPWDIEPQFGDKIEELDHHFNNRLRIR | |
| | YNNFTAPFNFAQLNP* | |
| Psychromonas | MAENKKKVRKSSSKVIAKPKVIYAITAGGLQDLGNLVGINKSDIYT | 139 |
| ingrahamii 37_gvpF | IEKESISFVVSDLSPSSPRPRPDRRNIMAHNEILKQLMSKTSVLPVRF | |
| | GTVATGERAVNRFCSQYNAQLLEQLDRVQDRVEMGIKVTWNVP | |
| | NIYDYFVDNHSELREERDRVYDGNKNPRRDDRINLGHMYDALVT | |
| | EARLSHQTDLEEIILPGCDEIHSIPPKDEKVVVNLACLVQRADLEVF | |
| | EERVVEAGKTLDNTYDIELNGPWAPHNFVELDLKTMTGRR* | |
| Serratia sp. ATCC | MMSIDKSRNHRAKVLYALCVSDDSTPNYKIRGLEAAPVYSIDQDG | 140 |
| 39006_gvpF | LRAVVSDTLSTRLRPERRNITAHQAVLHKLTEEGTVLPMRFGVIAR | |
| | NAEAVKNLLVANQDTIREHFERLDGCVEMGLRVSWDVTNIYEYF | |
| | VATYPVLSETRDEIWNGNSNANNHREEKIRLGNLYESLRSGDRKE | |
| | STEKVKEVLLDYCEEIIENPVKKEKDVMNLACLVARERMDEFAKG | |
| | VFEASKLFDNVYLFDYTGPWAPHNFVTLDLHAPTAKKKTLTRAG | |
| | TLSD* | |
| Stella | MQTEALAPAAVAAEGKYLYCIIDAPAPATFASPGIGGRGDVVHTL | 141 |
| vacuolata ATCC- | AVGRLAAVVSDTPRIEYENSRRNMMAHTKVLEEVMAHHTLLPVC | |
| 43931_gvpF | FGTVGSGDDVIAEKILEGRREELSRLLEEMRGRVELGLKATWREE | |
| | VIFAEVLDEDPAVRKLRDSLVGRSPEKSHFERIRLGELIGQALLRKR | |
| | RDEEERILDRVRPFVRKTKLNKPIGDRMILNAAFLVETAREAALDQ | |
| | SVREMDADWGARLSFKYVGPVPPYNFVTITIHW* | |
| Thiocapsa rosea strain | MQQAKRQDVAAGRYIYAIIPDRGDHSLGRIGLDESEVYTIGDGRV | 142 |
| DSM 235 | AAVVSDLSGGRIRPQRRNMAAHQEVLKQVLREVSPLPAAFGLMA | |
| Ga0242571_11_gvpF | DDEAAIIRILKDNQDAFLNQLERVDGSLEMGLRMSWDVPNIFEYF | |
| | VGAHPELQELRDDFFRDGSNLTQDQMITLGRSFERLLEQDREEYTE | |
| | QVESVMRSCCREIKRNKCRTEKEVLHLACLVDRDAAGRFEQVVL | |
| | QAARPFDNNYAFDFNGPWAPHNFVEMDIHV* | |
| Tolypothrix sp. PCC | MDAGLYLYGIFSDPIPPTVSLKGLDSQPVYSQVIEGFTFLYSDAKQE | 143 |
| 7601_gvpF | KYLASRRNLISHEKVLEQAMQEGFRTLLPLRFGLVVKNWETVISQ | |
| | LIQPCERQLRDLFQKLAGKREVSVKILWDTKAELQAMMQSNPDL | |
| | KQKRDQMEGKNLSMEEVIEIGQLIESNLQQRKEAVIKTFFDELKPL | |
| | AEEVVESEPMMEEMIYNAAFLIPWDQEALFSQRVEAIDKKFGDRL | |
| | RIRYNNFTAPYTFAQIS* | |
| Trichodesmium | MEFGFYVYGLIQEKGKMDESKDESKNGLKGSNESKDELKGLDKE | 144 |
| erythraeum | DVKIQDVDEFAVLYSIAKKERYLASRRNLITHEKVLESAMEAGYR | |
| IMS101_gvpF | NLLPMQFGLVVSEWEKFSQDFTKPCEQQIHDLFTKLKNNREVGIKI | |
| | YWEPDAELEKLLENDKDLKEERDSLKDKKLTMDQVIDIGQKIEQG | |
| | MNERKQNIIEIFQETLNKMAIEVIENEVQTEKMIYNAAYLIPWDQE | |
| | EDFGEKVETIDSKLCERGNFTIRYNSFTAPYNFARIRQQD* | |
| gvpF/L | | |
| Ancylobacter | MTDLLVFAVVPADRFDPAILAEGDGLPPGLRAIAAGPLAAVVGAA | 145 |
| aquaticus strain | PEGGLKGRERSALLPWLLASQKVMERLLANAPVLPVALGTVVED | |
| UV5_gvpFL1 | EGRVRHMLDAGAAILGEGFQAVGDGIEMNLSVLWHLDTVVARLL | |
| | PGVAPELRQAAAGGDAIERQALGVVLAGLVSAERRRARARVIEAL | |
| | QAVTRDFAIGEPTEPGGVVNLALLVDRAAEEALGAALEALDAEFD | |
| | GALTFRLVGPLPPYSFASVQVHLSPAAAVCGARAALGVEPDASPE | |
| | TVKAAYRRAARETHPDLVPMGGEDEEAPEATADETSRFVVLSDA | |
| | YRVLEGEHAPVSLRRLDSVLTE* | |
| Ancylobacter | MLYVYAITADYAAGANHLLPAKGIVPGVPVQRFGTGALGAVASP | 146 |
| aquaticus strain | VPVTVFGKEALHALLDDADWTRARILAHQRVVSSLLPLATVLPLK | |
| UV5_gvpFL2 | FGTLVAGEASLAAALTSQHDALDATVARLRGAREWGVKLFFEAP | |
| | TRTIRAEEPVGAGAGLAFFRRKKEEQETRAAAEAALDRCVAASHR | |
| | RLASHARAAVANPLQPPELHGHPGTMGLNGAYLVAAENEAAWR | |
| | VCFSELEQAYAALGARYVRTGPWAAYNFTGGGLV* | |
| Aquabacter spiritensis | MSGLLVFAIVPADRIEPGLLAPAEGLPPGLETVVAAGFAAIVGTAP | 147 |
| strain DSM | EGGLKGRDRGSLLPWLLASQKVIERLMARGPVLPAALGSVLEDES | |
| 9035_gvpFL1 | RVRHMLVCGQAALAAAFETLNGCWQTDLSVRWDLSRTVAHLMT | |
| | ELPPGLRAAAETGDETARRSLGAALAGLVAGERRRIQSRIGAVLG | |
| | AVARDLIVSDPVEPEGVVGVALLVDAPASAQVDAALDRIDGEFE | |
| | GRLTFRLVGPLAPYSFATVQIHLGPAAGLAGAHAELGLEAGAPLE | |
| | AVKAAYHRLIVGLHPDLVPHGSPGDDADDAASGKGGRAARFAAV | |
| | TAAYRTLQAEHAPVSLRRQDGLSPG* | |
| Aquabacter spiritensis | MLYVYAITADHPGPHDAGSLPGEGIVPGAPVRLLPFGDLAAAVSP | 148 |
| strain DSM | VSAVDFGPEALPARLQDVDWTGQRVLAHQRVVDSLVDVATVLP | |
| 9035_gvpFL2 | MKFCTLFSGAAALRAALADNRAALEATVVRLRGAREWGVKLFW | |
| | EAPPAEPAPVERGPGAGAAFFQRKRDAQRLRAEAEAALAHGVAE | |
| | SHRRLAARARAAVANPVQPAAVHRRRGEMALNGAYLVPRADEA | |
| | AWRESLAELERTYAGAGIRYELTGPWGPYNFTGGGLAGS* | |
| Bradyrhizobium | MTMNLVGITTPDVAGAIAAAGGRLADVETRAVEAGGLVALLALS | 149 |
| oligotrophicum | KAPFWHVLRRSRTALRSMLTAQRILEAAAVYGPLLPARPGTLIRN | |
| S58_gvpFL1 | DAEACMLLRSQCRHLAEGLRLHGTSRQYQITISWDPVAALAARRD | |
| | HQDLVEAAAASADGAADKAASMIQRFMSDQQARFEAEAMRALA | |
| | AVAEDVITLPVNQPDMLMNAVVLLAPGAEPELERVLEALDRGLR | |
| | GKNLIRLIGPLPPVSFAAVSIERPGRQRIAAARRLLGIGEATRTCDLR | |
| | RAYLDKAHAHHPDTGGHAADASIVGAAAEAFRLLARVAEARASA | |
| | GQDDVILVDIRRQDQQRSLST* | |
| Bradyrhizobium | MSKANLGIGLVHGVVTAQSAALLPQIVDAFDATEIIVVNTEQQALL | 150 |
| oligotrophicum | ISDIPQYLRGHVEADTLFSDPARISTLAMKHHRILQAAAVVTDVVP | |
| S58_gvpFL2 | VRLGTLVRGPSGARDLLNREAVRFAGHLVTIHNALEFSVRILPTEQ | |
| | PSRRVARPVPSSGRDYLRIRRDERCGQRPAVVDITLQELASRAVAI | |
| | RERQSASRSGGRTPALAEAAFLVDRHALAAFDDCAGRIERQIAEN | |
| | GLALDIFGPWPAYSFVDGARENLG* | |
| Bradyrhizobium | MSSPRLIGLLAADDVPADLADQIMSCGPVAAAIRFAPAAASSSESL | 151 |
| oligotrophicum | DHHAAVVAWCRRAAFLPSRAGIPISPELLQSIARSAWYHRSTIEHIE | |
| S58_gvpFL3 | GRVEISVELERRDGVRDGGIDGGGRAYLRATAHDLRACEVGVAT | |
| | AANLLAMYSERADADLIARTAPLPAIRLRASVLVRRAVAPRLARQ | |
| | FDSMLSAISDRLVCRVTGPWPPYSFSTIREPS* | |
| Burkholderia | MVWLTYAVLTPKRSITLPPGVAGARLEIVDGAHLRTIVSEHPRAPS | 152 |
| thailandensis sp. | ATIPSALDFGQTVAALFRHGAIVPMRFPTCLDSKQAVRDWLDDES | |
| Bp5365 strain | DMYRDLLQRIDGCVEMGLRFRLPEAPRAQPRPQAGGPGHAYLAA | |
| MSMB43_gvpFL | RGAPNSVARSHGERIAAVLRNLYRDWRFDGLVEGFVSLSFLVRQT | |
| | TLDDFVDRCRQAARETAFPLYMSGPWPPYSFATDERSSAPEPHRA | |
| | LRLMRRPSTAVSISANVAAPEKKDSAR* | |
| Desulfobacterium | MTLHLLYCVFSSGEMEKTRKLVPPGIDGEPVHEICSNKISGVVSTL | 153 |
| vacuolatum-DSM | GKPPDTHVKSLLAYHGVIDSYHQNRTVIPMRFAAVFRTYAHMITA | |
| 3385_gvpFL | LNNNEKSYLLQLKRLHDCTEMCVRFISNSPCCVKKKEPAISPKKIS | |
| | GTTFLQQRKAMYEQQNRLPPEIHEKTRDILQHFRGLYMEFKQESQ | |
| | PLEKDCPSLSLQGAEKTDGNALLISLFFLISKKNISLFRSRFQNICGS | |
| | SSGRHMMNGPWPPFNFINTESNLTDPS* | |
| Desulfomonile tiedjei | MLGSLAAIQFLSISSYGADEMKFLMYCIFTENSIEPPHSLVGVNRSP | 154 |
| DSM 6799_gvpFL | VRIISCDGLAAAVSVITQKEIPRDPATGLDYHKVIQWFHERIGVIPL | |
| | RLGTCLGHESDVVQLLHSHGARYKSLLKELDGCVEMGIRVIHDRP | |
| | GPQELASKSPFISRFNGTESGTDYLMRRKVLFDADEFAISRNREIVE | |
| | RYHSPFTGLYVSFKAQTSKFSPLGTDRNSVLTSLYFLIPRQSADSFR | |
| | AIYGDLRSGLHERIMLSGPWPPYNFVLPEDCL* | |
| Enhydrobacter | MEGHRIYIYGIVRDAADGGPAPVPPVAGLDGGALRAIAGYGLAAI | 155 |
| aerosaccus strain | ASAVDLSKAGIPFEEQLKDPDRATALVLEHHRVLQQAIDAQTVLP | |
| ATCC 27094_gvpFL | MRFGALFQDDRGVTDALEKNRCGLMDALGRIDGAREWGVKIFCD | |
| | RAVAARQLSATSAVVQAAEKELSGLAEGRAFFLRRRLERLRTEET | |
| | DRAVAHEVDVSRQALCELARASAPLKLQPAAVHGRGEDMVWNG | |
| | AFLVPRSGEERFLSRLEVVVQSRSDLGLHYEVTGPWPPFSFVDGQL | |
| | EGGGDACPDGA* | |
| Octadecabacter | MRSATSIVYAYGVLTNCSDIALDMPRSDLAGLVKNGPLRILPFGNI | 156 |
| antarcticus 307_gvpFL | AAVVCDFVLPNGSDLETLLEDSRSAERLILNHHQVLSYIVSQHTILP | |
| | LRFGAAFTEDAGVIAALGGRCSELQKALGRIDGALEWGVKTFCDR | |
| | KLLKQRVRGTGSEISDLESEIAKQGEGKAFFLRRRKERLILEEVEEI | |
| | LEQCVVGTQEQLEPSVIEEALVKLQPPTVHGHEHDMLSNISYLIAR | |
| | GTEDAFMQSLEDLRLAHAPYGLEYQMNGPWPAYSFSDQQLEGGV | |
| | NDQ* | |
| Octadecabacter | MSSATSIVYVYGVLTNCSDLVLDFPPGDLAGIVESGPLRILPFGDIG | 157 |
| arcticus 238_gvpFL | ALVCDFILPDGSDLKTILEDSRSAERMILNHHLVLADMVSRYTILPL | |
| | RFGAVFAEDAGVIAALGGRYSTLQKELDRIDGAIEWGVKSFCNRK | |
| | MFSECVAETVSEISVLEKEIADQGEGKAFFLRRRIQRLILDEVEKTL | |
| | EQCLVGAQDQLKSRAIEETLVKLQPPTVHGHKHEMVSNRSYLIAR | |
| | GAEDAFMQSLDDLRVVYAPFGFDYQINGPWPAYSFSDQQLGGGV | |
| | NDK* | |
| Rhodobacter | MGHYLYGLLAPPARGTLAQMQAAAAGVTSLGGPVALSAVEGML | 158 |
| capsulatus SB | LVHCPCDLAEISQTRRNMLAHTRMLEALMPLATCLPVRFGVIAQD | |
| 1003_gvpFL1 | LAEVARMIHERRAELVGHAQRLLDPVEIGLRVRFPRDRALAQLMA | |
| | ETPDFVAERDRLMGQGAGAHFARADFGRRLAEALDARRTRDQKR | |
| | LLAALRPHVRDHVLRAPEEDVEVLRAEFLIPAAGVDAFSRIAHDLA | |
| | AALGFAGAAEPELQVIGPAPPYHFLSLSLAFDNTSEAA* | |
| Rhodobacter | MAHEIIAILPCEAAQLPSGLTGVVGRGATAVLAPAPGWAERLTGG | 159 |
| capsulatus SB | PKQTAVRHHSRLEALMAMGSVLPFAAGIACTPEEAALLLRLDAPLI | |
| 1003_gvpFL2 | ARLAAEIGPRRHFQLALDWDESRVLAAFRDSPELAPLFSGAAVTPE | |
| | ALRQAITALADRLSATALRLLDPVAEDPVEQPRAPGCLLNLVFLLR | |
| | PEDEPRLDAALQAIDALWSEGLRLRLIGPSAPISHALVDIDRADVA | |
| | ALAAAADLLKVAPEAGPEAVTEAAKAALRSPDLAANAAEQIRAA | |
| | ARLLLRAGDIAALGLSGAATLPHLVHLRPGGRKSGLTSSGEAA* | |
| Rhodobacter | MTGLALHGFVSPDGWSAAAAPPARCAVVLGGVAALVSEAGDAL | 160 |
| capsulatus SB | DTPETAQAAALAHHALISAWHRRGPVLPVRLGTVFSSQAALQTAL | |
| 1003_gvpFL3 | APKAAQLRAALDALADKEEMVLTIVPAARPPDLPPPAATGADWL | |
| | RARKAVRDRGQARQTDRQQTLAGLQDALRAQGVASLAAPAPRE | |
| | GGSRWHLLIARDDGAGLDRWLAAQADRFDAAGLDLTLDGPWPP | |
| | YRFAAEILEALDG* | |
| Rhodobacter | MSEPRISGLAPWRADLPDVIGCHGGWVLMGAAADETPEARLRRQ | 161 |
| capsulatus SB | VGWCRAAVDVLPLSPRLAPTRAEAERLVATRGPDLERAHRHIRGR | |
| 1003_gvpFL4 | LQVIVQLEMCRTDLGLVRREISGGRSWLQDRAERATREARANADF | |
| | EAQVRRVVRALFPREGQVVTLAPSGTAGQLRLRRAVLVPRAGLQ | |
| | AFAAALSADLDRDGRGGLWDVIAPLPPLAFAALEAGPGGAVT* | |
| Rhodobacter | MIYLYGLLEEPASGHEVLAGMAGVTGPIALARLPGGILIYSSATEA | 162 |
| sphaeroides | DILPRRRLLLAHTRVLEAAAWFGNLLPMRFGMMASTLAEVAAML | |
| 2.4.1_gvpFL1 | ASRLTELCAAFDRVRGRVELGLRLSFPREPALAATLATAPDLAAER | |
| | ARLLALRRPDPMAQAEFGRRLAERLDARRGETQRLLFQSLRPLWV | |
| | DHRLRVPDSDVQVIAVDVLVEDGAQDRLAAALVKAAADCSFAPT | |
| | AEPSVRVIGPVPLFNFVDLVLSPRREEVA* | |
| Rhodobacter | MRLREVVAVLEGHPPSVLPEGTEAICEAGLTAILGMPPGLLSGRRA | 163 |
| sphaeroides | LLEHAACRQAVLERLMAFGTVLPVLTGNCLTPAEAAAALAANSP | |
| 2.4.1_gvpFL2 | RLRQELRRLAGRVQFQVLVQWHAALVPKRTDPDETAEDLRLRFT | |
| | HRIADALARVAERHVNLPLREDMLANQALLLLQTRTDDLDRSLEQ | |
| | IDALWTEGLRIRRIGPSPPVSFASLNFRRVSSAAIRRARHRFDLEGP | |
| | VDPIRLRALRRDLLLRASEAERAEILAAAAVLDLLTRCAASGGDLH | |
| | LVRIWSEGQAVPSDLEDAA* | |
| Rhodobacter | MSGLLLLGVVSGLGISPAITSPHLRLDGDGYAAILLSLDRLPPDPAS | 164 |
| sphaeroides | PDWAVQAALAQNAILSAYAATEDVLPVALGAAFTGIAAVKRHLD | |
| 2.4.1_gvpFL3 | AERATLDAGMERLAGRAEYVAQLIAEQVADGAAPAPASGSAFLK | |
| | ARSARHEQRRHLARERTGFARATAEELASLSCSASARPLKPDGPLL | |
| | DLSLLVARDRVPGLLEAAEASSRAGSRLALSVRLIGPCAPFSFLPET | |
| | RGHD* | |
| Rhodobacter | MAGDARSRVRLHLAAMRDCETFLPFPPAATIAVDEAIAWCGRRTN | 165 |
| sphaeroides | ALAEEIDRFSRQRQLTVSARLIAPLLPDAAASGAGWLRARRDASA | |
| 2.4.1_gvpFL4 | HQARLRTVLMQIMSLLGEVRCIPGRLQDEVQVNLLVPAAETHPVL | |
| | HELRERLRVGDALWSACTVTGPWPPYAFISWETA* | |
| Rhodococcus hoagii | MSEQESAPDGGGPVVYVYGLVPADVEVKEDATGIGSPPRPLKIVH | 166 |
| 103S_gvpFL1 | HEDVAALVSEIDPDTPLGSSDDLRAHAAVLDSTATVAPVLPLRFG | |
| | AVLTDTDAVVAELLEPYRDEFHEALEQLEGKVEFVVKGKYVEDAI | |
| | LREILADDPEAARLRDVVREQPEDTTRDERLALGERISQALTAKRE | |
| | QDTGRIVEALQPAATAVAPREPTDDEEAGSVAVLISADGVDELDK | |
| | AVARLIDDWQGRVEVTVTGPLAAYDFVKTRAPGT* | |
| Rhodococcus hoagii | MTPDDGVWVYAVTGDGSFPGGISGIRGVAGEELRTVTDSGFTAVV | 167 |
| 103S_gvpFL2 | GTVRLDTFGEEALRRNLEDLDWLADTARRHDAVVAAICAGGATV | |
| | PLRLATVYFDDDRVRTMLRDNAEQLGEALQQIADRSEWGVRAYL | |
| | ERPRSEPRDAREKTGRPSGTAYLMQRRAQVAAREQAESAAGRRA | |
| | DEIFAELARWAVAGVRQPPSPPDLAGRRSQEILNTSFLVDNGRHRE | |
| | FVTAVEELDARLSDVDLVLTGPWPPYSFTSVEASAR* | |
| Serratia sp. ATCC | MSLLLYGIVAEDTQLALEPDGSPHAGEEPMQLVKAATLAALVKPC | 168 |
| 39006_gvpFL | EADVSREPAAALAFGQQIMHVHQQTTIIPIRYGCVLADEDAVTQH | |
| | LLNHEAHYQTQLVELENCDEMGIRLSLASAEDNAVTTPQASGLDY | |
| | LRSRKLAYAVPEHAERQAALLNNAFTGLYRRHCAEISMENGQRTY | |
| | LLSYLVPRTGLQAFRDQFNTLANNMTDIGVISGPWPPYNFAS* | |
| Stella vacuolata- | MSGLLVFAIVPADGIEPGILAPREELPANLRAVAADGFAAVVGAAP | 169 |
| ATCC-43931_gvpFL1 | EGGLKGRDRSVLLPRLLASQKVIERLMARGPVLPVTLGTVLEDEA | |
| | RVRHMLAAGAPMLEAAFGTLGDCWQMDLSVRWDLNQVVARLM | |
| | GEVPGDVRAAAGSGDEAARRALGEALAGLAAGERRRVQSRLAA | |
| | ALRDVARDLIVSEPVEPESVVDIAILVERPALAEVEAALDRLDAEF | |
| | EGRLKFRLVGPLAPHSFATVQVHLAPEAALAGACAELGVERGAGL | |
| | QDVKVAYHRALVRFHPDLAPHGDDGGPEDEHDGGEGRASRLLTV | |
| | TAAYRALQAEHAPISLRRQDGIAVNQEQDASAAMGQQRGIVPGRE | |
| | LQALRM* | |
| Stella vacuolata- | MLYVYAIAADHPDPDNAMFGGEGIVPDAPVRLLQLGDLAVAASL | 170 |
| ATCC-43931_gvpFL2 | VSAADFAADALRAHLEDARWTALRVLAHQRVVDSLLPHATVLP | |
| | MKFCTLFSGEAALKQALAHNRAALQATVERLRGAREWGVKLYW | |
| | EAPRNPAPPSAGQGEAGAGAAFFQRKRDQQRQRAEAEAAVARCV | |
| | AASHRRLADAARAAVANPVQPPAVHRQPGEMALNGAYLVARAA | |
| | EPAWREVLAELERTHADGGIRYELTGPWGPYNFTGSGLVGS* | |
| Thiocapsa rosea strain | MSDRPRPMLHCILRSPPGSIARAEAGLRWIERDGLAALVADREPSE | 171 |
| DSM 235 Ga0242571- | IAGASSVGLQRYADIVAEIHACAAVIPVRFGCLLAGDEAVGKLLHR | |
| 11_gvpFL | SRDRLHGLLDQVGDCLEFGIRLLLPADAPAATDDDAAPRLHANAP | |
| | SDPRADPDMGPGLSHLLAIRHRLDVEASLAARAREAREVIKGRVA | |
| | GRFREVREELGQIDGRSLLSLYFLVPREQGEHFVECLRQDASSLRG | |
| | TGLLTGPWPPYNFVGAIDDDIRSLD* | |
| gvpG | | |
| Anabaena-flos- | MLTKLLLLPIMGPLNGVVWIAEQIQERTNTEFDAQENLHKQLLSL | 172 |
| aquae_gvpG | QLSFDIGEIGEEEFEIQEEEILLKIQALEEEARLELEAEQEEARLELEA | |
| | EQEDFEYPPQFTAEVNKDQHLVLLP* | |
| Bacillus- | VLHKLVTAPINLVVKIGEKVQEEADKQLYDLPTIQQKLIQLQMMF | 173 |
| megaterium_gvpG | ELGEIPEEAFQEKEDELLMRYEIAKRREIEQWEELTQKRNEES* | |
| Ancylobacter | MGMLTDVVFAPAVGPLKGVLWLARIIAEQAERTLYDEGVIRAALL | 174 |
| aquaticus strain | DLEQQLEAGEIDEDAYETQETVLLERLKIARERMRSGL* | |
| UV5_gvpG | | |
| Aphanizomenon flos- | MLTKLLLLPIMGPLNGLVWIGEQIQERTNTEFDAQENLHKQLLNL | 175 |
| aquae NIES-81_gvpG | QLSFDIGEISEEDFEIQEEELLLKIQALEEEARLELELAEEEARLELEL | |
| | EQEEEEDFVVKPQLTTEIDRDKDLVLLP* | |
| Aphanothece | MVFKLLLLPITGPIEGVTWLGEQILERANQELDEKENLNKRLLSLQ | 176 |
| halophytica (strain | LSLDLGEISEEEYDEQEEEILLAMQAMEDEENNQAEEETD* | |
| PCC 7418)_gvpG | | |
| Aquabacter spiritensis | MSLVTDVLFAPAVGPLKGVLWLARLIAEQAERTLYDEDVLRAAL | 177 |
| strain DSM | LDLEQRFEAGEISEADYETEEDILLARLKIARERMRSGL* | |
| 9035_gvpG | | |
| Bradyrhizobium | MLFQILTSPVSGPFRMVSWIGGAIRDAVDTKMNDPAEIKRALAAL | 178 |
| oligotrophicum | EQQLEAGSLSEQDYERMEMELIERLQSSLRHGSGNGG* | |
| S58_gvpG | | |
| Burkholderia | MFILDNLLAAPIKGMFWIFEEIAQAAEEETIADIEMIKAALVELYRE | 179 |
| thailandensis sp. | LESGQIDETEFETRERALLDRLDSLETS* | |
| Bp5365 strain | | |
| MSMB43_gvpG | | |
| Chlorobium luteolum | MFILDDILLAPLSGMVFLGRKINEIVQNEMSDEGAVKEQLMKLQF | 180 |
| DSM 273_gvpG | RFEMDELSEEEYDRLEDELLSTLAEIRAQKENR* | |
| Dactylococcopsis | MVFKLLLLPITGPIEGITWLGEQILERADQELDSKENLNKRLLSLQL | 181 |
| salina PCC | SLDLGEISEEEYDEQEEEILLAMQAMEDEENEEEES* | |
| 8305_gvpG | | |
| Desulfobacterium | MFLVDDILFFPAKSLVWVFRELHNAVQQEKTNESDALTTELSELY | 182 |
| vacuolatum DSM | MMLETGKITEEEFDEREEQILDRLDEIQERDQ* | |
| 3385_gvpG | | |
| Desulfomonile tiedjei | MERYTMFLLDDILFLPMNGVLWICNEIHDAAEQELHNESDAITAQ | 183 |
| DSM 6799_gvpG | LQKLYTLLEAGDIGESEFDVLEAELLDRLDAIQERGALLEA* | |
| Desulfotomaculum | MLGKLLLSPILGPVMGVKFIAEKIKQQADQELYDKSKIKQDLMEL | 184 |
| acetoxidans DSM | QIKLELEEITEEYYLQREEELLVRLDELASMETEEEEV* | |
| 771_gvpG | | |
| Dolichospermum | MLTQLLLLPIMGPLNGVVWIAEQIQERTNTEFDAQENLHKQLLSL | 185 |
| circinale_gvpG | QLSFDIGEISEEEFEIQEEEILLKIQALEEEARLELEAEQEEARLELEA | |
| | EQEQARLELEAEQEELENQPQLTPKIDTYRHLVKL* | |
| Enhydrobacter | MGMLARLLTLPVSAPVGGVLWIARKIEEEANAERWDRNKITGALS | 186 |
| aerosaccus strain | ELELELDLGAIDVEEYDAREAVLLQKLKELQEVEND* | |
| ATCC 27094_gvpG | | |
| Isosphaera | MFLVDDILLAPAHSLMFLLREIHQAALEELRRDAQKVREELAECY | 187 |
| pallida_ATCC- | RALETGALTDEEFASLETDLLDRLDALEELARFNSDEDDDPEDED | |
| 43644_gvpG | WDVEDDDPAEAVW* | |
| Legionella drancourtii | MLLLGSILMAPVHGLMAIFEKIKEAVDEEKQHDIERIKSELMALYT | 188 |
| LLAP12_gvpG | KLESGELSEADFEKQEKILLDKLDSLEDEDD* | |
| Microcystis aeruginosa | MFLDLLFLPVTGPIGGLIWIGEKIQERADIEYDEAENLHKLLLSLQL | 189 |
| NIES-843_gvpG | SYDMGNISEEEFEIQEEELLLKIQALEEEEAENESESSL* | |
| Nostoc punctiforme | MVLRFLLLPITGPLMGVTWLGEKILEQASTEIDDKENLSKQLLALQ | 190 |
| ATCC 29133_gvpG | LAFDMGEIPEEEFEIQEEALLLAILEAEQEERDQTQEY* | |
| Nostoc sp. PCC | MLGKILLLPVMGPINGLMWIGEQIQERTNTEFDAQENLHKQLLSL | 191 |
| 7120_gvpG | QLKFDMGEISEEEFDIQEEEILLKIQALEAEERLNAESEEDDDLDVQ | |
| | PIFILASEENPVYQDQSRFSEEYEDKEDLVLSP* | |
| Octadecabacter | MGIILNTLMSPLIGPMKGVFWVAEQIKDQTDAEIYDDSKILVELSE | 192 |
| antarcticus 307_gvpG | LELLLDLEKIELKDFEAKEDVLLKRLQEIRKAKKNDSV* | |
| Octadecabacter | MSIILNTLMGPLIGPMKGLLWVAEQIKDQADAELYDDSKILVALSE | 193 |
| arcticus 238_gvpG | LELSFDLEQIELKEFEAQEDVLLQRLQAIRKAKQNDTD* | |
| Pelodictyon | MFILDDILFAPLNGLIFIAKKINDVVEKETSDEGVVKERLMALQLRF | 194 |
| phaeoclathratiforme_ | ELDEIDEVEYDREEDELLQKLERIRLNKQNQ* | |
| gvpG | | |
| Phormidium tenue | MLFKLLFAPVLGPIEGISWVANKLLEQADVPTNDLESLQKQLLAL | 195 |
| NIES-30_gvpG | QLAFDMGEVAEADFEIQEEEILLAIQAIEDEEDEDE* | |
| Planktothrix agardhii | MILRLLLSPITAPFEGVIWIGEQLLERAEAELDDKENLGKRLLALQL | 196 |
| str. 7805_gvpG | AFDMGDIPEEDFEVQEEELLLQIQALEDEANQENDEID* | |
| Psychromonas | MFILDDILLAPYSGIKWLFKEIQRQAQEELDGEADRITTDLTNLYR | 197 |
| ingrahamii 37_gvpG | QFESNEITEQEFEERETVLLDRLDELQEESNLLDEEYDEEYEDDDE | |
| | EYEDDDEEYEDDDEEYEDDDEEYEDDDKNDKDKNDDHDNDDDD | |
| | ENKDENDKYNDEER* | |
| Rhodobacter | MGLLRKLLLAPVELPITGALWIVEKIAETAESELTDPGTVRRLLRG | 198 |
| capsulatus SB | LEQQLEAGEITEEEYEFAEEILLDRLKRGQAAEARSGGP* | |
| 1003_gvpG | | |
| Rhodobacter | MGLLTSLLTLPFRGPFDGTLWIAARIGEAAEQSWNDPAALRAALV | 199 |
| sphaeroides | EAERQLLAGELSEETYDAIELDLLERLKGTAR* | |
| 2.4.1_gvpG | | |
| Rhodococcus hoagii | MGLFSAIFGLPLAPVRGVVWIGEVVRRQVEEETTSPAAMRRDLEAI | 200 |
| 103S_gvpG | EEGRRSGEISEDEAAQAEDEILHRVTRRRDAGASGEE* | |
| Serratia sp. ATCC | MLLIDDILFSPVKGVMWIFRQIHELAEDELAGEADRIRESLTDLYM | 201 |
| 39006_gvpG | LLETGQITEDEFEQQEAVLLDRLDALDEEDDMLGDEPGDDEDDEY | |
| | EEDDDEEDDDEEDDDDEDDDDEDDDDEEDDDDDEDDDDEDEPE | |
| | GTTK* | |
| Stella | MGLVTNVAFAPVVGPLKGVLWLARLIADQAERTLYDEDLVRAAL | 202 |
| vacuolata ATCC- | LDLEQRLDAGQISEADYDAEEEILLARLKIARERMRSGL* | |
| 43931_gvpG | | |
| Thiocapsa rosea strain | MLIVDDLLAAPFKGIIWVFEEIHKSATAEQRARRDEIMAALSALYR | 203 |
| DSM 235 | ALEQGEITDDTFDTREQALLDELDALDAREDANELGSDEDEDDLD | |
| Ga0242571_11_gvpG | GAGEDAS* | |
| Tolypothrix sp. PCC | MEVMIMLGKILLFPVMGPISGLMWIGEQIQERTDTEFDAQENLHK | 204 |
| 7601_gvpG | QLLSLQLSFDIGEISEEDFEEQEEELLLKIQALEEEKARLEAESIEDE | |
| | EDEVEPTYFIAEVEEDKVLAEAFRGNKKYEDNENLVLSP* | |
| Trichodesmium | MLLRLLTLPISGPLEGVTWLGKKLQEQVDTEIDETENLSKKLLTLQ | 205 |
| erythraeum | LAFDMGEISEEDFEDQEEELLLAIQALEEQKLKEEEEDA* | |
| IMS101_gvpG | | |
| gvpJ | | |
| Anabaena-flos- | MLPTRPQTNSSRTINTSTQGSTLADILERVLDKGIVIAGDISISIASTE | 206 |
| aquae_gvpJ | LVHIRIRLLISSVDKAKEMGINWWESDPYLSTKAQRLVEENQQLQ | |
| | HRLESLEAKLNSLTSSSVKEEIPLAADVKDDLYQTSAKIPSPVDTPI | |
| | EVLDFQAQSSGGTPPYVNTSMEILDFQAQTSAESSSPVGSTVEILDF | |
| | QAQTSEESSSPVVSTVEILDFQAQTSEESSSPVGSTVEILDFQAQTSE | |
| | EIPSSVDPAIDV* | |
| Bacillus- | MAVEHNMQSSTIVDVLEKILDKGVVIAGDITVGIADVELLTIKIRLI | 207 |
| megaterium_gvpJ | VASVDKAKEIGMDWWENDPYLSSKGANNKALEEENKMLHERLK | |
| | TLEEKIETKR* | |
| Ancylobacter | MNEQRMEHSLQAVGLADILERVLDKGIVIAGDITISLVEVELLNIRL | 208 |
| aquaticus strain | RLVVASVDRAMSMGINWWQSDPHLNSHARELAEENKLLRERLDR | |
| UV5_gvpJ1 | LEAAVVPSALPADAALEPSLAGEDARHGG* | |
| Ancylobacter | MPSRHSGEIAVADLLDRALHKGLVVWGEATISVAGVDLVYLGLK | 209 |
| aquaticus strain | LLLTSTDTVNRMREAANAPPDERHLHAD* | |
| UV5_gvpJ2 | | |
| Aphanizomenon flos- | VTSTPILPTRPQTNSSRAINTSTQGSTLADILERVLDKGIVIAGDISISI | 210 |
| aquae NIES-81_gvpJ | ASTELIHIRIRLLIASVDKAKEMGINWWETDPYLSTKAQRLVEENQ | |
| | QLQNRLENLESQINLLTSAKVQEQISLVETTEDNTHQTTEDNTHQT | |
| | HEESIPLPIDSQLDV* | |
| Aphanothece | MVNPNTNKPKSYQSKGITNSTQSSSLADILERVLDKGIVIAGDITVS | 211 |
| halophytica (strain | VGSTELLSIRIRLLVSSVDKARELGINWWEGDPYLSSQANLLKEEN | |
| PCC 7418)_gvpJ | QALQNRLENMEAELRRLKGETNPEPSFLSESEDNS* | |
| Aquabacter spiritensis | MSEQRMEHSLQAVGLADILERVLDKGIVIAGDISISLVEVDLLNIRL | 212 |
| strain DSM | RLVVASVDRAMSMGINWWQSDPHLNSHARQLEEENRLLRERLDR | |
| 9035_gvpJ1 | LEAALAPPEGGMLRAEVEVAHGG* | |
| Aquabacter spiritensis | MPDPEPIIPRTSGDVALADLLDRALHKGLVLWGEATISVAGVDLV | 213 |
| strain DSM | YLGLKVLLASTDTANRMRDAAAASAAGSHLPGG* | |
| 9035_gvpJ2 | | |
| Arthrospira platensis | MTLQSRSSSPQRGVPMSTSGSSLADILERVLDKGIVIAGDISVSVGS | 214 |
| NIES-39_gvpJ | TELLSIRIRLLIASVDKAKEIGINWWESDPYLSSQAQQLSQSNQQLL | |
| | EEVKRLQEEVRSLKALTSQSSQPVTPPNSENDD* | |
| Bradyrhizobium | MTFTVHQPTGGDRLADILERVLDKGIVVAGDVTISLVGIELLNIKIR | 215 |
| oligotrophicum | LIVATVDRALELGINWWEADPRLTTRASELSVENEELKKRLALLE | |
| S58_gvpJ1 | ADAGRNQRPRKRRVRSIAATSGASHER* | |
| Bradyrhizobium | MTYRADLDYLEPAASSEGSLLELLDHLLDRGVLLWGELRISVADV | 216 |
| oligotrophicum | ELIEVGLKLMLASARTADRWRQTTTQRASIAPGDCP* | |
| S58_gvpJ2 | | |
| Burkholderia | MRSADGEPVSAELAQRLSLCESLDRILNKGAVISAQVVVSVADVD | 217 |
| thailandensis sp. | LLYLHLRLLLTSVETALVGRAMPREEASR* | |
| Bp5365 strain | | |
| MSMB43_gvpJ1 | | |
| Burkholderia | MADLLERVLDKGVVITGDIRINLVDVELLTIRIRLLVCSVDKAKEL | 218 |
| thailandensis sp. | GIDWWNADTFFLGPDRGQSALPGRASAVDVAAGSAVHADAAHR* | |
| Bp5365 strain | | |
| MSMB43_gvpJ2 | | |
| Chlorobium luteolum | MPELKHAVNATGLADILERVLDKGIVIAGDIKIQIADIDLLTIKIRL | 219 |
| DSM 273_gvpJ1 | MVASVDKAIEMGINWWQEDPYLSTGAKTSEQTRLLGEINQRIEKL | |
| | ESINR* | |
| Chlorobium luteolum | MQEDLYTANRQVTLLDILDRVLNKGVVISGDIIISVAGIDLVYVGL | 220 |
| DSM 273_gvpJ2 | RVLLSSVETMERLDAARAEGLQQ* | |
| Chlorobium luteolum | MAVEKTIGSSSLVEVIDRILDKGVVVDAWVRVSLVGIELLAIEARV | 221 |
| DSM 273_gvpJ3 | VVASVETYLKYAEAIGLTAKAA* | |
| Chlorobium luteolum | MAVEKTIGSSSLVEVIDRILDKGVVVDAWVRVSLVGIELLAIEARV | 222 |
| DSM 273_gvpJ4 | VVASVETYLKYAEAIGLTAKAA* | |
| Dactylococcopsis | MVNSNTNQPKSYQSKGITNSTQSSSLADILERVLDKGIVIAGDISVS | 223 |
| salina PCC 8305_gvpJ | VGSTELLTIRIRLLISSVDRAREIGINWWESDPYLSSQAHLMKEENQ | |
| | ALQSRLENMEAELRRLKGETNLDQSSLGESDQRSLQ* | |
| Desulfobacterium | MAYIDIDNDASKQISICEALDRVLNKGAVITGELTISVADIDLIYLSL | 224 |
| vacuolatum_DSM | QAVLTSVETARHMFDSQINDAVKEVK* | |
| 3385_gvpJ1 | | |
| Desulfobacterium | MPIQRTAQHSIESTNIADLLERVLDKGIVIAGDIKISLVDIELLSIQLR | 225 |
| vacuolatum DSM | LVICSVDKAKEMGMDWWVNNPVFMPNKGTQNDEIADTLTKINSR | |
| 3385_gvpJ2 | LEHLEKATISGS* | |
| Desulfomonile tiedjei | MMDEEEHVSLCEALDRVLNKGAVIAGEVTISVANVDLIYLGLQVV | 226 |
| DSM 6799_gvpJ1 | LASVDTIRGKRNELLRHDVGLHLTADNA* | |
| Desulfomonile tiedjei | MSIQASTRHSIQSTNLADLLERVLDKGVVIAGDIKIKLVDVELLTIQ | 227 |
| DSM 6799_gvpJ2 | IRLVVCSVDKAKEMGMDWWTNNPAFQPALAQISE* | |
| Desulfotomaculum | MGPQMGPIKSTGNLSLLDVIDRILDKGLVINADISVSIVGVELLGIKI | 228 |
| acetoxidans DSM | KAAVASFETAAKYGLQFPTGTEINEKVSEAAKQLKEICPECGKKSG | |
| 771_gvpJ1 | RDELLHEGCPWCGWISARALRLETEHSQR* | |
| Desulfotomaculum | MLPIREERATLTDLLDRVLDKGLLLNADILISVAGVPLIGITLKAAI | 229 |
| acetoxidans DSM | AGMETMKKYGLLIDWDQESRLAERRLRSSRH* | |
| 771_gvpJ2 | | |
| Enhydrobacter | MAVTNGRMEHSIQGSSLADILDRILDKGIVIAGDVTISLVGVELLNI | 230 |
| aerosaccus strain | RLRLLVASVDKAIEMGINWWEADPYLTSQTKASSEQTELLQQRLE | |
| ATCC 27094_gvpJ1 | RIEGLLAGQATKEQPL* | |
| Enhydrobacter | MPVQTAHDGELALADLLDRALNKGVVLWGDATISLAGVELVYV | 231 |
| aerosaccus strain | GLRVLVASCSTMEKYRSSPRKGSMPIARGES* | |
| ATCC 27094_gvpJ2 | | |
| Isosphaera | MIVCSSSTPERIGPPMNLPPPHHAPWCYDSPDLETLPLDPAERIALC | 232 |
| pallida ATCC- | EVLDRVLNKGVVIHGEITISVAGVDLVYLGLNLLLTSVETAQSWK | |
| 43644_gvpJ1 | FRGMIE* | |
| Isosphaera | MAITRSSRPDVTHSTSGATLADVLERVLDKGLVIAGDIKIKLVDVE | 233 |
| pallida_ATCC- | LLTIQIRLVVASVDKAREMGLDWWTRSPELSSLAATTCPALTPPKQ | |
| 43644_gvpJ2 | EATPPATRIQAPTESAQTTPDQSHPSDPSASNIDEVAELRRHIELMQ | |
| | LRDEARQRAHREELAALRAQLTRLTELLDSPR* | |
| Legionella drancourtii | MIIEDKPVSLCETLDRVLNKGVVVAGTVTISVADVDLLYLDLHCL | 234 |
| LLAP12_gvpJ1 | LSSMKGMNLIGSERER* | |
| Legionella drancourtii | MELQKSPTHSIGSTTIADLLERILDKGIVIAGDIKVNLVQVELLTIQI | 235 |
| LLAP12_gvpJ2 | RLLICSVDKAKEIGMDWWTHQNDVQSKNGSMPIQEYVTQMEERL | |
| | KNLENTLASSKNAI* | |
| Lyngbya confervoides | MTGQSLSRSSSANRQMATATQGSTLVDVLERVLDKGIVIAGDISVS | 236 |
| BDU141951_gvpJ | VGSTELLTIRIRLLVASVDKAREMGINWWENDPYLSARSQELLTA | |
| | NEQLQSRIESLEQELKSLRSQED* | |
| Microcystis aeruginosa | MTSSTFAGSLRNQSNNSLKTATQGSSLADILERVLDKGIVIAGDISV | 237 |
| NIES-843_gvpJ | SIASTELINIRIRLLIASVDKAREMGINWWEGDPYLHSQSQALLAEN | |
| | RELSLRLQTLETELETLKSLTQLSAMESHDTSPNDEAHSSDA* | |
| Nostoc punctiforme | MSTNTNRGAITTSTQGSTLADILERVLDKGIVIAGDISISVGSTELLN | 238 |
| ATCC 29133_gvpJ | IRIRLLISSVDKAKEIGINWWESDPYLNSQTRTLLATNQQLQERLAS | |
| | LETELQSLKALNPINHQNAGD* | |
| Nostoc sp. PCC | MTTTPIHPTRPQTNSNRVIPTSTQGSTLADILERVLDKGIVIAGDISIS | 239 |
| 7120_gvpJ | IASTELIHIRIRLLISSVDKAREMGINWWENDPYLSSKSQRLVEENQ | |
| | QLQQRLESLETQLRLLTSAAKEETTLTANNPEDLQPMYEVNSQEG | |
| | DNSQLEA* | |
| Octadecabacter | MNDGKMEHSLNATNLADILERVLDKGIVIAGDVTISLVGVELLNIK | 240 |
| antarcticus 307_gvpJ1 | LRLLIASVDKAMEMGINWWAHDPFLTAGAQAPAVADPAMLERM | |
| | DRLEAALATALASNQTTPMKGHK* | |
| Octadecabacter | MTNKAQGGQDLALADLLDRALSTGVVIWGEATISLAGVDLVYVG | 241 |
| antarcticus 307_gvpJ2 | LKVLVASVDAAERMKAASLVDRPTDRGQQI* | |
| Octadecabacter | MNNGKMEHSLDATNLADILERVLDKGIVIAGDVTISLVGVELLNIK | 242 |
| arcticus 238_gvpJ1 | LRLLIASVDKAMEMGINWWAHDPYLTAGAQAPVGVDPAMLERM | |
| | DRLEAALAKALASNQTTPAEGQSS* | |
| Octadecabacter | MTNETQGGQDLALADLLDRALSTGVVIWGEATISLAGVDLVYVG | 243 |
| arcticus 238_gvpJ2 | LKVLVASVDAAQRMKDASLVDRPTDGGQ* | |
| Pelodictyon | MPELKHAVNATGLADILERVLDKGIVIAGDIKIQIADIDLLTIKIRLL | 244 |
| phaeoclathratiforme_ | IASVDKAMEMGINWWQEDTYLSTKAKDKEQQLLRDDLQQRIEKL | |
| gvpJ1 | EALTKIT* | |
| Pelodictyon | MQDEFYSKNKEITILDVLDRVLTKGVVITGDIVISVADIDLVYVGL | 245 |
| phaeoclathratiforme_ | RLLLSSVETMEKNKQNSIKM* | |
| gvpJ2 | | |
| Phormidium tenue | MATATQGSSLVDVIERVLDKGIVIAGDISVSVGSTELLSIRIRLIISSV | 246 |
| NIES-30_gvpJ | DKAREIGINWWESDPYLSSRTNELLEANQQLQSRLETLEAELKALR | |
| | SAEPVS* | |
| Planktothrix agardhii | MNSQQLPSNIQRGVPTSTQGSSLADILERVLDKGIVIAGDISVSVGS | 247 |
| str. 7805_gvpJ | TELLNIRIRLLIASVDKAREIGINWWESDPYLSSQTKVLTESNQQLL | |
| | EQVKFLQEEVKALKALLPQENQPNPISDPHK* | |
| Planktothrix | MNSQQRPSNIQRGVPTSTQGSSLADILERVLDKGIVIAGDISVSVGS | 248 |
| rubescens_gvpJ | TELLNIRIRLLIASVDKAREIGINWWESDPYLSSQTKVLTESNQELL | |
| | EQVKLLQEEVKALKALLPQENQPKEME* | |
| Psychromonas | MANVQKSTDSSGLAEVVDRILEKGIVIDAFVKVSLVGIELLSIEARV | 249 |
| ingrahamii 37_gvpJ1 | VIASVETYLKYAEAIGLTASAATPA* | |
| Psychromonas | MPMANVSINPELTAQECEKISLCDALDRIINKGVVIHGEITISVANV | 250 |
| ingrahamii 37_gvpJ2 | DLISLGVRLILSNVETREQSNTPKEEV* | |
| Psychromonas | MATGKPQSMTHSVKSTTVADLLERILDKGIVVTGDIKIKLVDVELL | 251 |
| ingrahamii 37_gvpJ3 | TVELRLVICSVDKAVEMGMDWWNNNPAFAPQAPAQEGELSSIEK | |
| | RLEKIEKALVK* | |
| Rhodobacter | MGYRSASQPEGLADVLERILDKGIVIAGDVSVSLVGIELLTIRLRLL | 252 |
| capsulatus SB | IATVDKAREMGIDWWSHDPYLNGRLRPGEPAPETETETAALRDRL | |
| 1003_gvpJ1 | AQLEAQLSALGAQVGAAPALAEPALRGLAAAGSSALCAAPEASSA | |
| | DVVQPVFRRYKEAP* | |
| Rhodobacter | MDDRFSLRLFGPEEVFDAPSGGLADLLDGLLGHGIVLHGDLWLTV | 253 |
| capsulatus SB | ADVELVYVGLSAVLASPEALRSHE* | |
| 1003_gvpJ2 | | |
| Rhodobacter | MSFQMQSPLQQDSLADVLERILDKGIVIAGDISISLVGIELLTIRLRL | 254 |
| sphaeroides | LVATVDKAREMGINWWESDPRLCITQAPASDGSAALLDRLERIET | |
| 2.4.1_gvpJ1 | QIGQLAAAREG* | |
| Rhodobacter | MTDSAPTLQFATAEEALQSSETRLVDVVDALLSQGIAIRGELWLTI | 255 |
| sphaeroides | ADVDLVFLGLDLLLANPDRLQCRVPDAA* | |
| 2.4.1_gvpJ2 | | |
| Rhodococcus hoagii | MTRSGSGANYPQQYSQGLGGAGHEPANLGDILERVLDKGIVIAGD | 256 |
| 103S_gvpJ | IRVNLLDIELLTIKLRLVIASLETAREVGIDWWEHDPWLSGNNRDL | |
| | ELENERLRARIEALESGERRVADVTDPHRAVQPAESPAAEVRDDD | |
| | A* | |
| Serratia sp. ATCC | MPVNKQYQDEQQQVSLCEALDRVLNKGVVIVADITISVANIDLIYL | 257 |
| 39006_gvpJ1 | SLQALVSSVEAKNRLPGRE* | |
| Serratia sp. ATCC | MSGNKKLTHSTDSTTVADLLERLLDKGVVISGDIRIRLVEVELLTL | 258 |
| 39006_gvpJ2 | EIRLLICSVDKAVEMGLDWWSGNPAFDSRARVSSSAPAPELEERL | |
| | QRLEARLEAAPSVIEETHL | |
| Stella | MSGQRMEHSVQAVGLADILERVLDKGIVIAGDISISLVEVELLTIRL | 259 |
| vacuolata ATCC- | RLVVASVDRAMSMGINWWQSDPNLNSHARQLEEDNRLLRERLDR | |
| 43931_gvpJ1 | LEAALALPEMAGERLADAGQGGGAEQGVTHGR* | |
| Stella | MSDPEPIIPRTSGDIALADLLDRALHKGLVLWGEATISVAGVDLVY | 260 |
| vacuolata ATCC- | LGLKVLVASTETADRMRAAAASQSADPKVRAG* | |
| 43931_gvpJ2 | | |
| Thiocapsa rosea strain | MMLAIGEHPDCPEEIQRVSLCEALDRILNKGAVVSGELTIAVANVD | 261 |
| DSM 235 | LLYLSLQLVITSVETAKREMLYVRH* | |
| Ga0242571 11_gvpJ1 | | |
| Thiocapsa rosea strain | MSVQRSTLTHSTNSTSVADLLERVLDKGIVIAGDIRIKLVDIELLTIQ | 262 |
| DSM 235 | LRLVICSVDKAREMGIDWWSDNAMFKGLSSQASAASLPGTAAAS | |
| Ga0242571_11_gvpJ2 | GIEDRLARLESLLVKQSAAAETVL* | |
| Tolypothrix sp. PCC | MADILERVLDKGIVIAGDISVSIASTELLHIRIRLLISSVDKAKELGIN | 263 |
| 7601_gvpJ | WWENDPYLSSKSQRLVEENQQLQQRLESLEAQLRSLTAAKINNPE | |
| | LFPVNAEDNGQSDEENVPLPMNYQPND* | |
| Trichodesmium | MFIRVDFLLDKGVIVDAWVRLSLVVIELLTIEAKIVIASVEAYLKYS | 264 |
| erythraeum | EAFCFNY* | |
| IMS101_gvpJ1 | | |
| Trichodesmium | MAVEKVNSSSSLAEVIDRILDKGVVVDAWIRLSLVGIELLTIEARIV | 265 |
| erythraeum | VASVETYLKYAEAVGLTTLAAAPGEAAA* | |
| IMS101_gvpJ2 | | |
| Trichodesmium | MAVEKVNSSSSLAEVIDRILDKGVVVDAWVRLSLVGIELLTIEARI | 266 |
| erythraeum | VIASVETYLKYAEAVGLTTLAAEPAA* | |
| IMS101_gvpJ3 | | |
| Trichodesmium | MKTSANIATSASGNGLADVLERVLDKGVVIAGDISVSIASTELLNI | 267 |
| erythraeum | KIRLLISSVERAKEIGINWWESDPYFSSQNNSLVQANEKLLERVASL | |
| IMS101_gvpJ4 | ESEIKALRSN* | |
| Trichodesmium | MKTSANIAKSAGGDSLADVLERVLDKGIVIAGDISVSIASTELLNIK | 268 |
| erythraeum | IRLLISSVERAKEIGINWWESDPSLSSQNNSLVQVNQKLLERVASLE | |
| IMS101_gvpJ5 | SEIEALKYSQ* | |
| gvpK | | |
| Anabaena-flos- | MVCTPAENFNNSLTIASKPKNEAGLAPLLLTVLELVRQLMEAQVIR | 269 |
| aquae_gvpK | RMEEDLLSEPDLERAADSLQKLEEQILHLCEMFEVDPADLNINLGE | |
| | IGTLLPSSGSYYPGQPSSRPSVLELLDRLLNTGIVVDGEIDLGIAQID | |
| | LIHAKLRLVLTSKPI* | |
| Bacillus- | MQPVSQANGRIHLDPDQAEQGLAQLVMTVIELLRQIVERHAMRR | 270 |
| megaterium_gvpK | VEGGTLTDEQIENLGIALMNLEEKMDELKEVFGLDAEDLNIDLGPL | |
| | GSLL* | |
| Ancylobacter | MTAPCTAETLENALRGRIDIDPEKVEQGLVKLVLMLVETVRQVVE | 271 |
| aquaticus strain | RQAIRRVEGGTLTEEETERLGLALMRLEEKMAELRLHFGLEDGDL | |
| UV5_gvpK | DLKLQLPLGEL* | |
| Aphanizomenon flos- | MVYSPVENSNDFLNVIPVENSNEFLNTSPKKKSNSETGLAPLLLTV | 272 |
| aquae NIES-81_gvpK | LELIRQLMEAQIIRRMEEDLLSESDLERTAESLQKLEEQILNLCQIFD | |
| | IDPADLNINLGDFGSLLPASGSYYPGETGNRPSILELLDRLLNTGIV | |
| | VDGEIDIGVAQLDLIHAKLRLVLTSKPI* | |
| Aphanothece | MSADESNLSQVNLNPATSNSDAGLAPLLLTVTELIRQLMEAQVIRR | 273 |
| halophytica (strain | MDGGLLNEEELDRAGDSLQRLEAEIIRLCEIFEIDPKDLNVDLGELG | |
| PCC 7418)_gvpK | TLMPKNGGYYPGESSDDPSILELLDRILHKGVVIDGNLDLGIAQLS | |
| | LIQARLHLVLTSQPINGK* | |
| Aquabacter spiritensis | MTGFAGGPAVTETLESVLQGRVDIDPERVEQGLVKLVLMVVETLR | 274 |
| strain DSM | QVIERQAIRRVEAGALTDEEIERLGLTLLRLEEKMAELRVQFNLSE | |
| 9035_gvpK | ADLSLKLRLPLGEL* | |
| Bradyrhizobium | MSASSHSEAPGLRLQLGDLDTALAAVFTDAAPNGSINLDPDKIEHD | 275 |
| oligotrophicum | LARLVLTLIEFLRRLLELQAIRRMEANELSEDEEERVGLALMRAAA | |
| S58_gvpK | QVSRLARELGVDPRELNLQLGPLGRLL* | |
| Burkholderia | MNAPHAAAVSDAAALAAALEQALAQQQAPPPRATQRFDVATAS | 276 |
| thailandensis sp. | AGNGLAKLVLALMKLLHELLERQALRRIEAGSLNDDEIERLGLAL | |
| Bp5365 strain | MRQAEEIERLAAQFGFTDADLNLDLGPLGRLF* | |
| MSMB43_gvpK | | |
| Chlorobium luteolum | MHEDKVQFQASSVEEALRQLEGMKQGKESRIEANPDNVESGLAR | 277 |
| DSM 273_gvpK | LVLTLIELLRKLMEKQAMRRIDGGSLDEAQIDELGETLMKLEMKM | |
| | DELKKTFNLTDSDLNLNLGPLGDLM* | |
| Dactylococcopsis | MSEEESNLSRVDLNPASSNSDAGLAPLLLTVTELIRQLMEAQVIRR | 278 |
| salina PCC | MDAELLTEAELDRAGESLQRLEEEILRLCEIFDVDPADLNVHLGEL | |
| 8305_gvpK | GTLLPKEGGYYPGETSDQPSILELLDRVLHTGVVIDGNLDLGIAQL | |
| | NLIQAKLHLVLTSQPINN* | |
| Desulfobacterium | MIKDPEAKDFKIESDSIDAFARVMHADTSSCSSSSVTAGQRQQRLK | 279 |
| vacuolatum DSM | IDEENIKNGLAQLVMTLIKLLHELLERQAIRRIESGSLDDDQIERLG | |
| 3385_gvpK | LTLMQQCEEIDRLRKLFDLEEEDLNLDLGPLGKLL* | |
| Desulfomonile tiedjei | MNPMNIAKVESDSLGDFAEIMQTDWISSLHSDKEEKRLNLNQDSV | 280 |
| DSM 6799_gvpK | KNGLGQLVLTLVKLLHDLLERQAIRRMEAGTLTDTEIDRLGTTLM | |
| | MQAQEIERLRSEFGLEEEDLNLDLGPLGKLL* | |
| Desulfotomaculum | MYIDISEGSLKQGVLGLLLALVEIIKDALKIQALKRIEGDSLTEDEIE | 281 |
| acetoxidans DSM | RLGNALHELEEALVEIEMEHNLQNVVQNIREGLDNVVNEVVDTFN | |
| 771_gvpK | PERWIAENEFN* | |
| Dolichospermum | MLSTPADNFDESLTTVSKSKNEAGLAPLLLTVLELLRQLMEAQVIR | 282 |
| circinale_gvpK | RMEDNLLSESELERAADSIQKLEEQILHLCETFEVDPAELNINLGDF | |
| | GTLLPQSGSYYPGETGSRPSVLELLDRLLNTGVVLDGEIDLGLAQL | |
| | DLIHAKLRLVLTSKPI* | |
| Enhydrobacter | MTKLLEAKTVDPDKAGDDLVKLVLALVETLRQLVERQAIRRVDS | 283 |
| aerosaccus strain | GVLNDDEVERLGLALLRLEEKMSELKAHFGFGDEELTLKLGSLGE | |
| ATCC 27094_gvpK | LARDV* | |
| Isosphaera | MSDSLFEVRSPSAAPPSPVNPGVADEWTAVLKDWDTLTAQLRQA | 284 |
| pallida_ATCC- | TAPPNAENSARSHATTGRIDLDPEQVGDGLAKLVLTLLELIRQLLE | |
| 43644_gvpK | RQAIRRLDAGSLDHEQTERLGLTLMRLAQRMEELKTHFGLQGEDL | |
| | NLDLGPLGKLL* | |
| Legionella drancourtii | MNDKREEDNALPQRINLQPDDVKNGLGKLVLILIQLIHELLERQAI | 285 |
| LLAP12_gvpK | GRIEAGDLSDEQIDRLGITLMKQAEEIDKLREVFGLTQEDLNLDLG | |
| | PLGKLL* | |
| Microcystis aeruginosa | MTLACTPYDSDNQALLTRPESNSQAGLAPLLLTVVELVRQLLEAQI | 286 |
| NIES-843_gvpK | IRRMEKGVLSESDLDRAAESIQKLQEQILYLCEIFEVEPEELNVHLG | |
| | EFGTLLPEAGSYYPGEEGIKPSVLELVDRLLNTGVVVEGNVDLGL | |
| | AQLDLIHLKLRLVLTSQPV* | |
| Nostoc punctiforme | MQAISKSKGSDSGLAPLLLTVVELIRQLMEAQVIRRMDAGTLNDS | 287 |
| ATCC 29133_gvpK | ELDRAAESLQKLEQQVVQLCEIFDIDPADLNINLGEMGNLLPQSGG | |
| | YYPGETSSQPSILELLDRLLNTGVVVEGDLDLGLAQLSLVHAKLRL | |
| | VLTSKPL* | |
| Nostoc sp. PCC | MVCTPVEKSPNLLPTTSKANSKAGLAPLLLTVVELIRQLMEAQVIR | 288 |
| 7120_gvpK | RMEQDCLSESELEQASESLQKLEEQVLNLCHIFEIEPADLNINLGDV | |
| | GTLLPSPGSYYPGEIGNKPSVLELLDRLLNTGIVVDGEIDLGLAQLN | |
| | LIHAKLRLVLTSRPL* | |
| Octadecabacter | MKTTSDSQFDSMKKILTDSSKEDSASCDPTDLLPNKSLPPSLSTSPE | 289 |
| antarcticus 307_gvpK | TAADDLVKLVLAVIDTVRQVMEKQAIRRVESGALAEAEIERLGLT | |
| | LMRLEARMVELKSHFGLSNEDLNLHFGTVQDLKDILNDEE* | |
| Octadecabacter | MKTQNDTQFDSMKKILTDSGGGDPNPNGSPDQTQHASLPSNLSTD | 290 |
| arcticus 238_gvpK | PETAADDLVKLVLAVIDTVRQVMERQAIRRVDSGALADEEIERLG | |
| | LTLMRLEERMADLKSHFGLSNEDLNLNFGTVQDLKDILNDEE* | |
| Pelodictyon | MDSDKILYYAGSADEIIEELEKLKPGIQGRINATPDNVESGLAKLVL | 291 |
| phaeoclathratiforme_ | TLIELIRKLIEKQAMRRIDGNSLSESQIEELGETLMKLEKKMEELKG | |
| gvpK | IFNLTDKDLNLNLGPLGDLM* | |
| Phormidium tenue | MTSENAEPDLSTTLALQPPAKTDAGLAPLLLTVIELVRQLMEAQVI | 292 |
| NIES-30_gvpK | RRMESGDLDDNDLERAADSLRKLEEQVVSMCEIFDVDPADLNIDL | |
| | GEIGTLLPKEGNYYPGQKNQNPTILELLDRLLDTGVVVEGDVDLG | |
| | MAQLNLIHAKLRLVLTSKPI* | |
| Planktothrix agardhii | MSSSEPSIETIITPKSSRKDAGLAPLVLTLVELIRQLMEAQVIRRMEG | 293 |
| str. 7805_gvpK | NTLSEEELDRAAQSLQQLEIQVLKLCEIFEIDPTDLNIELSEFGTLLP | |
| | KSGSYYPGENTQNPSILELLDRLMNTGIVVEGSVDLGLAQLNLIHA | |
| | KLRLVLTSKPL* | |
| Psychromonas | MPFEHFKSNNQADVNSDTKPAASVGGLNLESDDLKNGLGRLVLT | 294 |
| ingrahamii 37_gvpK | LVKLLHELLERQALRRMDAGSLQDDEIERLGLAFMKQAEEIDRLR | |
| | KEFGLEVEDLNLDLGPLGRLL* | |
| Rhodobacter | MSAAMHLELGDVDAVLSQAARSLAAGGRLTLDPERVEQDLARLV | 295 |
| capsulatus SB | LGIVELLRKLMELQAIRRMEAGSLTPEQEETLGLTLMRAEAALHE | |
| 1003_gvpK | VAAKFGLQPADLILDLGPLGRSV* | |
| Rhodobacter | MTYPFPPLLLRDDRLPPTEAPVTAPRIALDPDRLEHDLARILLGLME | 296 |
| sphaeroides | MLRQIMELQAIRRMEAGSLSESQQEQLGTTLMRAEAAIHEMAARF | |
| 2.4.1_gvpK | GLTPADLSLDLGPLGRTI* | |
| Rhodococcus hoagii | MRRRIDSDPESVERGLVALVLTLVELLRQLMERQALRRVDAGDLS | 297 |
| 103S_gvpK | DDQIERIGTTLMLLEEKMEELREHFGLEPEDLNIDLGPLGPLLAED* | |
| Serratia sp. ATCC | MTTNQLSHHSPVFGPTSPAIQRPITEANRHKIDIDGERVRDGLAQL | 298 |
| 39006_gvpK | VLTLVKLLHELLERQAIRRMDSGSLSDEEVERLGLALMRQAEELT | |
| | HLCDVFGFKDDDLNLDLGPLGRLL* | |
| Stella | MTGFLNGPADVETLETALRGRVDIDPERVEQGLVKLVLMVVETLR | 299 |
| vacuolata ATCC- | QVIERQAIRRVESGSLTDDEVERLGLTLMRLEEKMDQLRRQFDLG | |
| 43931_gvpK | EEDLSMRLRLPLQEL* | |
| Thiocapsa rosea strain | MSDTRTGTAPSSAASAAPDTSTLQRANLLADLLETKVAAAGRRIDI | 300 |
| DSM 235 | DPERVQRGLGQLVLTVVKLLHVLLERQAIRRVDGGDLDEDEIEQL | |
| Ga0242571_11_gvpK | GLALMRQSEEIERLRRLLGLEEQDLNLDLGPLGKLF* | |
| Tolypothrix sp. PCC | MAMVCTPSENSNDLLATNSKANNQAGLVPLLLTVVELIRQLMEA | 301 |
| 7601_gvpK | QVIRRMEEECLSESDLERAAESLQKLEEQVLNLCQIFEIDPADLNIH | |
| | LGELGSLLPAAGSYYPGETGNTPSVLELLDRLLNTGVVVDGELDL | |
| | GVAQLNLIHAKLRLVLTSKPLNTK* | |
| Trichodesmium | MSLENSPEESLIVPIDKSKSNPEAGLAPLLLTVIELLRELMQAQVIR | 302 |
| erythraeum | RMDAGILSDEQLERAAEGLRQLEEQVIKLCKVFDIPTEDLNLDLGE | |
| IMS101_gvpK | IGTLLPKSGEYYPGEKSENPSVLELLDRILNTGVVLDGTVDLGLAE | |
| | LDLIHARLRLVLTA* | |
| gvpL | | |
| Ancylobacter | MLYLYAILESPPPQKPLPPGIGGAAPLFVESHALVCAASEAADAAI | 303 |
| aquaticus strain | AREPSQIWRHQEVVAALMEGRPVLPLRFGTVVEDSAACLRLLARH | |
| UV5_gvpL | HAELSAQLDRVRHCVEFALRVAGLSELADPGLDPNATPAGLGPGA | |
| | SHLRTLVRRERGWPVSSAAFPHDTLTAHAASRLLWARSPSQPDLR | |
| | ASFLVQRRSASAFLDDVNALQRLRPDLGITVTGPWPPYSFSDPDLS | |
| | GGRE* | |
| Aphanothece | MLYTYCFLFSPEKTLSLPQGFKGDLQMIEKGAIAAVVEPNLPKAEL | 304 |
| halophytica (strain | EEDDQKLVQAVVHHDWVICELFRGLTVLPLRFGTYFRGEADLRSH | |
| PCC 7418)_gvpL | LAAYEESYQQKLTALTGKVEVTLKLTPIPFSEEGSSSTAKGKAYLQ | |
| | AKKQRYQQQSNYQTQQQEALEKLQEEIKKTYPQLIHDEPKENTER | |
| | FYLLIDSHSFSVFGEKMEQWKQFLSSWSIEISDPLPPYHFL* | |
| Aquabacter spiritensis | MLYLYAVLEAPPPARSLPPGIGGGAPHFIEAFELVCAASETPNRSV | 305 |
| strain DSM | APEPAEVWRHQQVVEALIDRAPALPLRFGTLVEDASACRRLLTRH | |
| 9035_gvpL | RDALGAQLGRVRHCVEFALRVSGLPEEVAPDPGIGGGPGTSYLRT | |
| | LARREAGWPPSTAVFPHDGLAAHAAERLLWARSTSQPDLRASFLV | |
| | RKPNVAAFLADVSALQRVRPDLGITCTGPWPPYSFSDPDLSGVSP* | |
| Bacillus- | MGELLYLYGLIPTKEAAAIEPFPSYKGFDGEHSLYPIAFDQVTAVV | 306 |
| megaterium_gvpL | SKLDADTYSEKVIQEKMEQDMSWLQEKAFHHHETVAALYEEFTII | |
| | PLKFCTIYKGEESLQAAIEINKEKIENSLTLLQGNEEWNVKIYCDDT | |
| | ELKKGISETNESVKAKKQEISHLSPGRQFFEKKKIDQLIEKELELHK | |
| | NKVCEEIHDKLKELSLYDSVKKNWSKDVTGAAEQMAWNSVFLLP | |
| | SLQITKFVNEIEELQQRLENKGWKFEVTGPWPPYHFSSFA* | |
| Burkholderia | MNDALYLFCFARAEPLAPAWAKRAPGEPRLQLLHEGNLAAVLCD | 307 |
| thailandensis sp. | VSRSEFAGADAERRLADPAWIAGRVAVHAAAIEWTMRYSPVIPAQ | |
| Bp5365 strain | FGTLFSGAGRVIALMESCHAHIGRVLDHVEGKTEWAVKGWLDRQ | |
| MSMB43_gvpL | AAADSQAALLRADEPESAARTAGARYLRERQLQARAGQNLRDW | |
| | LEQSVPPISARLQRHAVEMCSRPCRASDSEHEIVANWAFLVRNRD | |
| | VPAFRRQAEAIDAEFATWGLHFDFSGPWPPYSFCAPLTEETTWSG* | |
| Chlorobium luteolum | MPCRLTVTWKSLRTAGLLPTAKGIQGRTERMAQNILYVYCIVRQL | 308 |
| DSM 273_gvpL | PGADIVARYPDLVFIEAGSAYVAAKYVSPLEYSDASMKLKLADEE | |
| | WLDRNAREHLSVNVMIMAQQTIIPFNFGTIFKSRESLSGFLGDYGR | |
| | KLDESFDALEGREEWAVKAYCNESFLLKNLHLESPAIAAIEQEIQA | |
| | ASPGKAYLLKKKKEAMSASALEGVHQGHAKAVWGELAALSKEH | |
| | VLNRLIPEDVSGVDGRMIVNGVFLIANTDVGAFIRTTEDLGERYRD | |
| | AGVFLDVTGPWPPYDFVDIPY* | |
| Dactylococcopsis | MLYTYCLIASSPSALSLPSGFRGELQLIKQGAIAAIVEAELPLEELEE | 309 |
| salina PCC 8305_gvpL | NDQKLIQAVIHHDAVICEIFQQIPLLPLRFGTYFPTEKDLLEHLDFK | |
| | AEKYQKKLQEIQDKVELTLKLTPLPFSTENASPMEKQGKNYLKAK | |
| | KQRYQEQTNYQSQQQAELNQLQTQINQDYPQFIHGEPKENIERFY | |
| | LLIKERDRSVFSEQLEQWKKDFPTWTIEVSDPLPPYHFIE* | |
| Desulfobacterium | MEKKKAVYLYCVTRANKFNAPGITGIDANTPVCFEHLENFVAVY | 310 |
| vacuolatum-DSM | NIIPLNTFVGTSAEENMKNIDWIGPRAMRHENVIERMMQESSVYPA | |
| 3385_gvpL | RFATLFSSMENLRETLHLKSGLISRFLNQTQHKCEYSLKGFINRKQ | |
| | LLEFLIKTKFKQEKKQLDGLSPGKKYFAQHQFNKKVETGINQWIK | |
| | RRCGIFLDHLTKRNPEVSPRELFTEKTEKNNLEMMENLAFLIHNDS | |
| | KSAFLQEISQAEKEFSQTGISLVVSGPWAPYSFCKTTRGEGL* | |
| Desulfomonile tiedjei | MSNVLYLFCLARTGLVDHIEGTGITGTEDLILKNFSGVTAVTCEVP | 311 |
| DSM 6799_gvpL | EDDFSGESAEIKLQDLAWVGPRAVRHDRIIEEIMQYSPVFPAPFGSL | |
| | FSSEKRLGTLIESNIDAIREFLDHTADKQEWSVKGLVCKSKAVDEIF | |
| | TGKLKILSETLSSSPAGMRYFKERQMRSEAEKELSGKVKAACTVV | |
| | GEKLLACSNNFRQRKNISFGKAEGDKQLVVNWAFLVDHSRISYFL | |
| | DQVEHANSNYQAGGLAFECSGPWPPYSFCPSLHMEPTR* | |
| Desulfotomaculum | MNLIDDCKAKYIYCIGENPGNWPSEVMGVEGSLVYHVVYRDIAA | 312 |
| acetoxidans-DSM | VVHDCAEQPYNSDDNNKVIDWVLGHQLVVDKACSCYSSVLPFTF | |
| 771_gvpL | NSIVKGKEDLSSHEILVNWLEDNYDNFKLKLGKIKGKKEYSVQLF | |
| | LDKQVSLSLLQSESDILELQVELLGSAKGKAYFVQEKINKKIGELM | |
| | ANRADSYCRQFYHEISSVVSECKLCKLKQAGRNEIMIINLVCLAGD | |
| | NEVEVLGDVLEKIKSNDIAIKIKFSGPWPAYSFV* | |
| Enhydrobacter | MLYVYGIADNAFEVLRGAGLLNSDVFAVPAGCLAAAASKLAQGG | 313 |
| aerosaccus strain | IETTPQGVWRHEQVLRQLMQDHAVLPLRFGTICRDRETLTDRLME | |
| ATCC 27094_gvpL | ASDDLVRGLGRVRGKVEIALRIVDEREHEAHPVPSETPTVDAIGGG | |
| | RGTAYLRARRRHHAAEMGREARAERVGKMLSAYIDVGAEDLVC | |
| | SVAPEGDHAVSVSCLLGRDQLATLQAALERFQSDHPAIGLSWTGP | |
| | WTPYSFVAPSLFGVGLP* | |
| Legionella drancourtii | MNKALYLFCLTPASDLPMMEGELLPNFSPLFIHPFQTFNAILSWVP | 314 |
| LLAP12_gvpL | AKEYQEQSTDSNLINTEEFMQRVFFHELVVEKIMRDEAVFPIGFGT | |
| | LFSSIASLEEQILTHQTLISSCLANLNQKDEYAVRVYLNQDKALESL | |
| | LSVMLQERESSWASSSPGVQYLKKQQLHNEIQRNLNQHLGGMLD | |
| | EVLSMFQRHATDFKSRENTAQSSDIHGTSILHWAFLIPRVVSSIFKE | |
| | QVDLMNAKYNPFGLHFVLTGPWPAYSFCTLQSVEAP* | |
| Lyngbya confervoides | MRWHRSEAVISYCDLSMIYLYALCPNSTETNNLPEGIGTAQVEVLT | 315 |
| BDU141951_gvpL | VGTLGAVIERDVDIAQIQKDDAQLMAAVLAHDRILSHLFTYSPLLP | |
| | LRFGTQFSNSEAVTTFLKTQGETYRQKLSHLQDRAEYLVKLIPQPL | |
| | DLPAIASDLKGREYFLAKKQRLQDHTAALNQQADELQTFLTDLAT | |
| | QDIPLVRSAPQDHEERLHVLLSRDTDTTEQVIMTWQEQLPNWQVV | |
| | CSEPLPPYHFAA* | |
| Octadecabacter | MKRLYVYGIVGATSFDDPLPNGHDEASVFALVSGDIAVAVSFVER | 316 |
| antarcticus 307_gvpL | SAVEASAANVWLHDNVLSALMTRYAVLPMRFGTIAVGATQLLEG | |
| | IVKRQKQLMKDLMRLNENVEIALHISGKNWEKVNQKVTKKNTDQ | |
| | AITQGTAYLLGRQQSLYGSDKTQLLVQNVRRAIRSGLDPLMKDVI | |
| | WPIDKPQALPFKASCLINRNDVASFVQIVNDIAAQNLDARVTCTGP | |
| | WAPYSFVGKSGVEGET* | |
| Octadecabacter | MTKLYVYGIVGATHFDVKLPNGHDEAPVFAIVSGDLAVAVSSLER | 317 |
| arcticus 238_gvpL | SAVEASAANVWLHENVLSALMEGHAVLPMRFGTIATGAAQLLGD | |
| | IVKRRGQLMKDLTRLDGKVEIALRISGKNREKVEQRIAGQIVDTNV | |
| | TQGVAYLQEKQQNLYGSFYTQSSVQCARRAIRSQLDPFIVEAIWPT | |
| | DEPQMLPFRASCLIKKGDIARFVQTVDDVVVKVSDIRVTCTGPWA | |
| | PYSFVGQSGSEAET* | |
| Pelodictyon | MVAIQERLIYIFCVTSEPPLLQQYQLQKGICVVDVDGLFVTTMDVT | 318 |
| phaeoclathratiforme_ | DNDFAENQLQSNLSDVVWLDTKVREHLDVITSIMQHVKSLIPFNF | |
| gvpL1 | GTLYKSESSLMQFIIKYAEEFKKNLVYLEEKEEWAVKLYCNKNKI | |
| | VENITHLSKKVSDINALIQNSSIGKAYILGKKKNEIIENEIINIYNTYS | |
| | KKIFTKFSILSEEFRFNPIPNNETLEKEDDMILNVVLLLNKANVESFI | |
| | ETSDQLIIQHQNIGLNIEITGPWPCYSFINISH* | |
| Pelodictyon | MPLIIYAIFDSINYIDSFSSYVDAISLKSKIKLEIISTSTLSAIVSRTTDE | 319 |
| phaeoclathratiforme_ | KKQACQNDVMIYATIIGDIAAKYSILPMRYGSIVSSPFDVTELLKN | |
| gvpL2 | HNETFVTIIKKITDKEEYSLRILYSHQDKEKNNIEDLFDLPQNVPDIL | |
| | HGNTDSKKYLLNKYIKHLSEEKRLQYIDKIQSIVACNLQKITDLIVY | |
| | NKQTTTGFIVDAVFMIERSKKSELLDLVIQMQTLFSEHNVVLSGPW | |
| | PPYNFSNINIG* | |
| Psychromonas | MKNSNHSGLDPNQALYLYCFVHADSIQSVTSQAIEKDSPVFIYQW | 320 |
| ingrahamii 37_gvpL1 | QDIAAVLSHVPTSYFTGYDDEEPEQTIARILPRTQLHEQVIEEVMRQ | |
| | SPVFPAQFGTLFSSQESLEQEISQQYLAITHTLKEVSGSVEWAVKG | |
| | VLDRGVAEKALYSQQLTEQQNSLSSSPGMRHLQEQRLRRETQSKL | |
| | NSWLHQLYTDIATPLSELSGDFFQRKIPSSIEEGKEVILNWAFLVPE | |
| | SAGDDFHAQIDKLNQRLNSFGLVIQCSGPWPPYSFCNQSS* | |
| Psychromonas | MKNSNHSGLDPNQALYLYCFVHADSIQSVTSQAIEKDSPVFIYQW | 321 |
| ingrahamii 37_gvpL2 | QDIAAVLSHVPTSYFTGYDDEEPEQTIARILPRTQLHEQVIEEVMRQ | |
| | SPVFPAQFGTLFSSQESLEQEISQQYLAITHTLKEVSGSVEWAVKG | |
| | VLDRGVAEKALYSQQLTEQQNSLSSSPGMRHLQEQRLRRETQSKL | |
| | NSWLHQLYTDIATPLSELSGDFFQRKIPSSIEEGKEVILNWAFLVPE | |
| | SAGDDFHAQIDKLNQRLNSFGLVIQCSGPWPPYSFCNQSS* | |
| Serratia sp. ATCC | MTMNTEAQTEQAIYLYGLTLPDLAAPPILGVDNQHPINTHQCAGL | 322 |
| 39006_gvpL | NAVISPVALSDFTGEKGEDNVQNVTWLTPRICRHAQIIDSLMAQGP | |
| | VYPLPFGTLFSSQNALEQEMKSRATDVFVSLRRITGCQEWALEATL | |
| | DRKQAVDVLFTEGLDSGRFCLPEAIGRRHLEEQKLRRRLTTELSD | |
| | WLAHALTAMQNELHPLVRDFRSRRLLDDKILHWAYLLPVEDVAA | |
| | FQQQVADIVERYEAYGFSFRVTGPWAAYSFCQPDES* | |
| Stella vacuolata- | MLYLYAVLEALPAARTLPAGIGGGELLFVEAFELVCAASETPERAI | 323 |
| ATCC-43931_gvpL | APEPTQVWRHQQVVEALIDCAAALPLRFGTLVEDAVACRRLLTRH | |
| | REALCAQLDRVRHCVEFALRVSGLREEVGSDHVIGGGPGVSYMR | |
| | ALARREASWPPSTGTFPHDGLAAHAADRLLWSRSASQPDLRASFL | |
| | VLKPNVAAFLADVSALQRMRPDLGITCTGPWPPYSFSDPDLSGMS | |
| | P* | |
| Thiocapsa rosea strain | MDAFYCFCFAPACLASDLRFDDCGWEDPIEIRRLAGLDVILSRVPL | 324 |
| DSM 235 Ga0242571- | GRFAGAEAEQRLADLEWLVPRAQAHDRVITRTMERSTVFPLTFAT | |
| 11_gvpL | LFSSLPALALEVAARRRALLDFFERMAGREEWAVKVSMDRERVIA | |
| | TRMQSLYPEGGDVPAGGRGYLLKQRRRGEAEQAIGPWLKGQIGC | |
| | LDEALRPSCETLLIRPLRDEMVASRACLVARDLGPSLSEAIERSREA | |
| | FADQGLDLHCSGPWPLYSFCGTP* | |
| Trichodesmium | MSYYVYGFLYLPESCLALPKGMEKEVELVPYQNIAAVVEANVSIE | 325 |
| erythraeum | AIQETEEKLLEAILAHDRVVREIFQQVSMLPLRFGNAFALRENIIND | |
| IMS101_gvpL | LQNNQQQYLNILTKLQQQAEYTITFTPVSYPSTLEVSKVRGKAYLL | |
| | AKKQQFEQQQAFQTKQRQQWENIRQLIFKNYPKAVFRDSTESKIK | |
| | QVHLLANRDARVITTEELSTWQTECSYWQITLSEQLPPYHFV* | |
| gvpN | | |
| Anabaena-flos- | MTTTKVNHKRAVLRLRPGQFVVTPAIERVAIRALRYLKSGFPVHL | 326 |
| aquae_gvpN | RGPAGTGKTTLAMHLANCLDRPVMLLFGDDQFKSSDLIGSESGYT | |
| | HKKVLDNYIHSVVKLEDEFKQNWVDSRLTLACREGFTLVYDEFN | |
| | RSRPEVNNVLLSALEEKILSLPPSSNQPEYLSVNPQFRVIFTSNPEEY | |
| | AGVHSTQDALMDRLVTISMPEPDEITQTEILIQKTNIDRESANFIVR | |
| | LVKSFRLATGAEKTSGLRSCLMIAKVCADNNIPVTTESLDFPDIAID | |
| | ILFNRSHLSMSESTNIFLELLDKFSAEELEILNNRVTGDNDFLIDNSQ | |
| | FVSQQLAGQPN* | |
| Ancylobacter | MTSEAASKDPISLLSGFGAGAASSGPKAGGRSTPSALTPRPRTGFV | 327 |
| aquaticus strain | EAEQVRDLTRRGLGFLNAGYPLHFRGPAGTGKTTLALHVAAQLG | |
| UV5_gvpN | RPVIIITGDNELGTADLVGSQRGYHYRKVVDQFIHNVTKLEETANQ | |
| | HWTDHRLTTACREGFTLVYDEFTRSRPETHNVLLGVFEERMLFLP | |
| | AQAREECYIKVHPEFRAIFTSNPQEYAGVHASQDALADRLATIDVD | |
| | YPDRAMELAVASARTGMPEASAARIIDLVRAFRASGDYQQTPTMR | |
| | AGLMIARVAAQEGFEVSVDDPRFVQLCSDALESRIFSGQRAEEVA | |
| | REQRRAALHALIDTHCPSAAKPRARRAGGAVRASIEGAQS* | |
| Aphanizomenon flos- | MTKTNHKRAVLRVRPGQFVVTPAIEQVAIRALLYLKSGFPIHLRGP | 328 |
| aquae NIES-81_gvpN | AGTGKTTLALHLAHCLDRPVMLLFGDDEFKSSDLIGSESGYTHKK | |
| | LLDNYIHSVVKVEDEFKQNWVDSRLTLACREGFTLVYDEFNRSRP | |
| | EVNNVLLSALEEKILSLPPSSNQPEYLSVSPQFRAIFTSNPEEYCGV | |
| | HSTQDALMDRLVTINMPEPDEITQTEILIQKTNIQKESAHLIVRLVK | |
| | SFRIATGAEKTSGLRSCLMIAKVCADNNLVAEPENSFFQEIAMEILS | |
| | NRTHLSVNESTDIFLDVISQFSNKEIEILNDAELGSLPTMDTLANTD | |
| | LGNDVPLEKEASDYVIQQKNNEFKGFQKPSTKVLN* | |
| Aphanothece | MTTVLHARPKGFVSTPTIDRISRRAWRYLQSGFSIHLRGPAGTGKT | 329 |
| halophytica (strain | TLAMHLADLLNRPIMLLYGDDEFKSTDLIGSNTGYTRKKVVDNYI | |
| PCC 7418)_gvpN | HSVVKEEDELRQQWVDSRLTMACREGFTLVYDEFNRSPPEVNNV | |
| | LLSALEEKLLVLPPDSHRSEYVRVSPNFRAIFTSNPEEYWGVHGTQ | |
| | DALLDRVVTINVPEPDLETQREIIVQKVGINADDGDMIVNFVRNFR | |
| | DRAEMENSSGLRSCLMIAQVCHQHEIPVQTSNEDFQDICYDILTSR | |
| | CPLSTQESISLLEQLFREYELELVVEDEDEDVPSVIVEGETEDLSSDE | |
| | KPHLRLSHPFGNTEND* | |
| Aquabacter spiritensis | MSTEPAPLVSPSQDVETTPQRPARPEPAEALAVGYRLSARPASPAT | 330 |
| strain DSM | LTPRPRADFVETDQVKDLTRRGLGFLRAGYPLHFRGPAGTGKTTL | |
| 9035_gvpN | ALHVAAQLGRPVIVITGDNELGTADLVGSQRGYHYRKVVDQFIHN | |
| | VTKLEETANQRWTDHRLTTACREGYTLVYDEFTRSRPETHNVLLG | |
| | VFEEKILFLPAQAREECYIRVHPDFRAIFTSNPQEYAGVHASQDAL | |
| | ADRLATIDVDYPDRGMELAVASARTGLGETEAARIIDLVRAFRAS | |
| | GDYQQTPTMRASLMIARVAAQEGLRVSIDDPGFVQLCMDALESR | |
| | MFSGARLEAATRETSRAALLALLAVHCPSEAPIVRVTAARRAKKA | |
| | DAS* | |
| Arthrospira platensis | MTTVLRAVPKGFVNTPAIERITVRALRYLQSGFSVHLRGPAGTGKT | 331 |
| NIES-39_gvpN | TLALHLADLLNRPIMLIFGDDELKSSDMIGNQTGYTRKKVVDNFIH | |
| | SVVKLEDSLKQNWIDSRLTLACREGFTLVYDEFNRSRPEVNNVLL | |
| | SALEEKLLVLPPNNSRSEYIRVNPHFRAIFTSNPEEYCGVYSTQDAL | |
| | LDRLITMNMPEPDEATQQEILIQKVAVTPEEAQTIVTLVQQFREAT | |
| | HAIAPSKIQTVARQQTNADKASGLRPSLMLARICQEHNIPIVPIDPD | |
| | FQEVCRDILLSRAIGDITELESRLHQIFDHLSGLENDQIIALPPREELT | |
| | TSSVPNNLSDTEQKIYTYIKDSDGARVSEIEIALGLNRVQTTDALRS | |
| | LLRKSYLTQQDNRLFVVYEGD* | |
| Bacillus- | MTVLTDKRKKGSGAFIQDDETKEVLSRALSYLKSGYSIHFTGPAG | 332 |
| megaterium_gvpN | GGKTSLARALAKKRKRPVMLMHGNHELNNKDLIGDFTGYTSKKV | |
| | IDQYVRSVYKKDEQVSENWQDGRLLEAVKNGYTLIYDEFTRSKPA | |
| | TNNIFLSILEEGVLPLYGVKMTDPFVRVHPDFRVIFTSNPAEYAGV | |
| | YDTQDALLDRLITMFIDYKDIDRETAILTEKTDVEEDEARTIVTLVA | |
| | NVRNRSGDENSSGLSLRASLMIATLATQQDIPIDGSDEDFQTLCIDI | |
| | LHHPLTKCLDEENAKSKAEKIILEECKNIDTEEK* | |
| Bradyrhizobium | MLRSDRAAIAGGQRGSRAQGDAVARNDAAAGSRAAIAQISPRPD | 333 |
| oligotrophicum | ADNAALSPAPRTDLFENPQLASMAARALTYLNAGIPVHLRGPAGT | |
| S58_gvpN | GKTTMAMQLAARLGRPVVLLTGDDGLTAAHLVGREIGTKSRQVV | |
| | DRYVHSVRRVETETSSMWCDAVLAQAVVEGLTFVYDEFTRSPPQ | |
| | ANNPLLSVVEERILIFPAGSRKERLVHAHPEFRAILTSNPEEYAGVS | |
| | RPQDALLDRLITFDLDDYDRETEIGIVSNRTGLAYAEAGVIVDLVR | |
| | GVRRWPKAHHPPSMRSAIMIARIVARELITPSVDDPRFVRLCLDVL | |
| | AAKAKPTDRDDRDRFAATLLRLMNNHCPAGAIDGG* | |
| Burkholderia | MEASAEFVQTPAVRNLTERALTYLGAGYGVHLAGPSGTGKTTLA | 334 |
| thailandensis sp. | FHIAAQLGRQVVLMHGDDELGSADLVGRGAGYRRSRVVDNFIHS | |
| Bp5365 strain | VVKTEEEMTTTWIDNRLTTACQHGLTLIYDEFNRSRPEANNALLP | |
| MSMB43_gvpN | VLSEGILNLPNRMTGAGYLTVHPGFRAIFTSNPEEYVGVHKTQNA | |
| | LMGRLITIQVGHYDRETEVEIVRARSGIARADAERIVDLTRRLRDA | |
| | DDNGHHPSIRAAIALARALSYCGGEATPDNAGYVWACRDILGVDL | |
| | EQDARTRSQAGRRTKARR* | |
| Chlorobium luteolum | MRAAVNDNEMNTVLAPRPMANFVETEYIRDITERGLTYLKAGFPV | 335 |
| DSM 273_gvpN | HFRGPSGTGKTTVAMHLAGKIGRPVVVIHGDSEYKTSDLIGSEQG | |
| | YKFRRLNDNFIHSVHKYEEDMSKQWVNNRLSIAIKKGFTLVYDEF | |
| | TRSRPEANNILLPILQEKMLSTSASNEEDYYMKVHPEFRAIFTSNPE | |
| | EYAGVNRTQDALRDRMVTMDLDYFDYETELRVTHAKSELTLEDS | |
| | EKIVQVVRGLRESGKTEFDPTVRGSIMIARTLHIMQVRPEKTNDAV | |
| | RKVFQDILTSETSRVGSKTNQEKVRAIVNDLIEAYL* | |
| Dactylococcopsis | MTTVLHARPKGFVSTPTIDRISGRAWRYLQSGFSIHLRGPAGTGKT | 336 |
| salina PCC | TLAMHLADLLNRPIMLLYGDDEFKSTDLIGSNTGYTRKKVVDNYI | |
| 8305_gvpN | HSVVKEEDELRQQWVDSRLTMACREGFTLVYDEFNRSPPEVNNV | |
| | LLSALEEKLLVLPPDSNRSEYVRVSPNFRAIFTSNPEEYWGVHGTQ | |
| | DALLDRVVTINVPEPDLETQQEIITQKVGINANDGEKIVNFVRQFR | |
| | DRAAVKNSSGLRSCLMIAQVCHQHEIPVQTSDEGFRDICYDILSSR | |
| Desulfobacterium | MSASMSSMKETRQRMSAPEQDNVVPEAGSDFVETPYVKDITDRA | 337 |
| vacuolatum DSM | LAYLHVGYPVHFSGPAGTGKTTLAFHVAAKLKRTVMLIHGDDEF | |
| 3385_gvpN | GSSDLIGKDSGYRKAKVVDNYIHSVVKTEESMNTVWADNRLTIAC | |
| | QQGCTLVYDEFTRSRPEANNAFLSVLEEKILNIPSLRDIDQGYLQV | |
| | HPEFRAIFTSNPEEYAGVHKTQDAMMDRLITITLDHFDRDTEVQVT | |
| | MSKSDLPQKDAEKIVDIVRKLRKTGVNNHRPTIRACIAIGKILKHM | |
| | GGGASKDNFVFKQICRDVLNVDTTKVTRDGEPLLPRKIDELINSL* | |
| Desulfomonile tiedjei | MNGAELRIASIETEVITANNENIVPEAGDRFVNTPHVEELTARAMA | 338 |
| DSM 6799_gvpN | YLEVGYSVHFSGVAGTGKTTLAFHAAAKLGRPVILVHGDHEFGSS | |
| | DLIGRDAGYKKSRLVDNFIHSVVKTEEEMRSLWVDNRLTTACRD | |
| | GYTLIYDEFTRSRPEANNVLLSILEEKILNLPSLRRTGEGYLEVHPSF | |
| | RAIFTSNPEEYAGVHKTQDALMDRIITINVDHYDRETEIEITRAKSG | |
| | VCKQDATVIVDIIRELRLLGVNNHRPTIRAAIAIARVLAHTGEHAD | |
| | QHNSVFQWLCKDVLSTDTVKVSRGGSPLMAKKVEEVIRKVCGRT | |
| | GGKRSGKPVGSKEETSE* | |
| Desulfotomaculum | MQLNGLDKNSIINPVVLSDFVVTDYISNVVDRALAYIKAGFAIHLR | 339 |
| acetoxidans DSM | GRSGTGKTSIAMYISSKLNRPTLVIHGDEEFRTSDLIGGRYGYRIRK | |
| 771_gvpN | TIDNFVQSVVKVEEDLVERWVDSRLTTACKNGYTLVYDEFTRSRP | |
| | EANNILLSVLQERLLDISVARGAEEGYVKVHPDFTAIFTSNPEDYA | |
| | GVYGSQDALRDRMVTLDLDNYDKETEISIIKSKSKLSREDSERVVN | |
| | ILRDLRELGDCEYGPTIRGGIMIAKTLQVLGAPVDKNNEMFRQICE | |
| | EVLASETSRAGNLQALRKVRKVINELFNKYA* | |
| Dolichospermum | MSITKVNHKRAVLRLRPGQFVVTPAIERVVIRALRYLRSGFPIHLR | 340 |
| circinale_gvpN | GPAGTGKTTLGMHLANCLDRPVMLLFGDDQFKSSDLIGSESGYTH | |
| | KKLLDNYIHSVVKVEDEFKQNWVDSRLTLACREGFTLVYDEFNRS | |
| | RPEVNNVLLSALEEKILSLPPSSNQPEYLSVNPQFRVIFTSNPEEYCG | |
| | VHSTQDALMDRLVTINMPEPDEITQTEILIQKTNIGRESANLIVRLV | |
| | KSFRLATGAEKTSGLRSCLMIAKICADHDIPASTEDLDFREIAIDILF | |
| | NRAQLSISESTDIFMGLLEQFSAEEIKVLNDTHFPTDELLINNSQFIT | |
| | QELVTQPNTELATDIPQELRKTEQN* | |
| Enhydrobacter | MSMDQAEEIGVVTTIEPRPRADFVRTQSVEATARRALGYLNAGFS | 341 |
| aerosaccus strain | VHFRGPAGTGKTTLALHLAALLGRPMVMITGDEEMLTSTLVGTQ | |
| ATCC 27094_gvpN | HGYHFRRVVDRFIHTVTKTEETADKRWADHRLTTACREGYTLIYD | |
| | EFTRSRPEANNVLLSVLEEGLLVLPAQNQNEPYIKVHPNFRVIFTSN | |
| | PQEYAGVHDAQDALGDRIVTIDMGHADRELELAIAAARSGLPPTQ | |
| | VAPIVDMVREFRETGEYDQTPTLRTSIMICRMMSQERLAPTIEDQQ | |
| | FVQICMDILGGKSLPGGKGDNKRAQQQKMLLSLIEHHCPARSFTS | |
| | VGEV* | |
| Isosphaera | MDYESTALQLKPRPDFVATPWVRELADRALGYLTAGYPVHFSGP | 342 |
| pallida_ATCC- | AGTGKTTLAMHLAALVNRPVVLLHGDDEFGSSDLVGDHLGFRST | |
| 43644_gvpN | KVVDNFIHSVVKTEQSVSKTWVDHRLTTACRHGFTLIYDEFNRSR | |
| | PEANNILLTILEERLLELPPIAGGRDGSGPLRVHPEFRAIFTSNPEEY | |
| | AGVHKTQDALLDRMITISMGGHDEATETEITAAKSGLSRDEAARI | |
| | VELARAVRALKPLRHPPTIRSCLMIAKVAALRKVPIDPNDALFLAI | |
| | CRDVLRIDALPVDDPEATFAELIRRVFAPTPAVAPPRVPTTGFAAN | |
| | RVVPIPRRPLAASASPPPGANGHAHLR* | |
| Legionella drancourtii | MMTQENNGSLTDSKNNDKLIRFVNNRSDNILLEASEEFTETPHIRGI | 343 |
| LLAP12_gvpN | SERALAYLDIGYPIHLLGPAGTGKTTVALHIAAQLGRPVILIHGDDE | |
| | FTGADLVGRGTGYHHSKLVDNFIHSVLKTEEEMTTMWTDNRLTT | |
| | ACEQGYTLIYDEFNRSRAEANNALLSVLSEGILNLPGRRERDGIGY | |
| | VDVHSNFRAIFTSNSEEYVGIHKTQNALADRLIAIKMDYPDQQSEI | |
| | QIIEKKSTLPRKDIEIIVNLARELRLKSEKRPSIRGCIAIARVLAYHNR | |
| | HAHADDPIFQAVCQDIFGISKEFLKQLLHPMDSGLQKRSEKNQESI | |
| | KKYKTKNQKL* | |
| Lyngbya confervoides | MSTVLQARPRNFVSTPAVERIARRALRYLQSGYSVHLRGPAGTGK | 344 |
| BDU141951_gvpN | TTLALHLADLLSRPIMLVFGDDEFKTSDLIGNQSGYTRKKVVDNYI | |
| | HSVVKVEDELRHNWVDSRLTLACREGFTLVYDEFNRSRPEVNNV | |
| | LLSALEEKLLVLPPSGHRPEYLRVNPHFRAIFTSNPEEYAGVHGTQ | |
| | DALLDRLITIHMPEPDELTQQQILIQKVGIEPADALMIVRLVKAFKS | |
| | QMGNHSATSLRPSLMIANICHEHGVAMMTEDADFRDVCSDVLLS | |
| | RVTNELSPATHTLWDLFNELTASADVLGPESNSTDVSPQPEADKP | |
| | VETKGSKGKSTTKSKAKESAKASEEADEAGDDSASAPELDEIESSI | |
| | LTFLTARESASLSEIESELSLTRFKAVDALRSLVEAGYLQKQNGAG | |
| | KPAIYGLVPEES* | |
| Microcystis aeruginosa | MTVTETQTRRAVLSLRPGQFVVTPSIDQIATRALRYLNSGFSIHLCG | 345 |
| NIES-843_gvpN | PAGTGKTTLAMHLANCLARPVMLIFGDDDFTSSDLIGSQSGYTHK | |
| | KLMDNYIHSVLKVEDELKHNWVDSRLTMACREGFTLVYDEFNRS | |
| | RPEVNNVLLSALEEKILTLPPTSHQPDYLQVNSQFRAIFTSNPEEYC | |
| | GVHATQDALMDRLVTINMPEPDQLTQTEILAQKTGIGREDALFIVN | |
| | LVKTFRVKTATEKTSGLRSCLMIAKVCASHDIAANSADSDFRDICA | |
| | DVLLSRTNLSVDKSRAILWEILEDNPLESLSFLEEEEPSDAQVSTSE | |
| | PSTGNQSLKAIQSLLRGNLPQRKD* | |
| Nostoc punctiforme | MTTVLNASPQRFVNTPAVQRIAQRALRYLQSGFSIHLRGAAGVGK | 346 |
| ATCC 29133_gvpN | TTLAMHLADLLNQPIILLFGDDEFKTSDLIGNQLGYTRKKVVDNFI | |
| | HSVIKVEDEVRQHWVDARLTLACKEGFTLVYDEFNRSHPEVNNV | |
| | LLSVLEERLLVLPTNQHRAEYIRVHPQFRAILTSNPQEYCGVHATQ | |
| | DALMDRVITIDMPTPDELSQQEIVVHKTGIDSEKAEVIVRIVRTFWS | |
| | RSGSGQGGGLRSCLMIAKICHEHEISVNPGDPSFQDICADILLSRTN | |
| | QPLIEATRLLEEVLSEFYHRINTQSQPSEIIPNNQNQIVLEQRVPYEH | |
| | EVYNYLCNSPGRRFSELAVELGIDRSQIVAALKSLREQGVLVQMQ | |
| | GNAESPSISQTVAFDSGHLINK* | |
| Nostoc sp. PCC | MTLTANNKKRAVLRVRPGQFVVTPAIEQVAIRALRYLTSGFAIHLR | 347 |
| 7120_gvpN | GPAGTGKTTLAMHLANCLDRPIMLIFGDDEFKSSDLIGSESGYTHK | |
| | KLLDNYIHSVLKVEDEFKQNWVDSRLTLACREGFTLVYDEFNRSR | |
| | PEVNNVLLSALEEKILTLPPSSNQPEYLHVNPQFRAIFTSNPEEYCG | |
| | VHSTQDALMDRLVTINMPEPDELTQTEILAQKTALNRADALLIVRL | |
| | VKAFRSRTGGEKTSGLRSCLMIAKVCAEHNILVSPQSSDFREICAD | |
| | VLFNRTNWSASEAATIFLELLNHLDLQQIEEFKNSIIPEDTDAIAEG | |
| | GFPTIIDSHFGTLDSEVLEQPGVEDSIPFEQEIYLYLQQYKSAALAL | |
| | VQQEFELSRTVATNALNSLEQKGLVSKNNHVYTIEEPNQS* | |
| Octadecabacter | MNSNLRATNSGGPDISKTMMPEAREDFVQTESVKSISRRALAYINA | 348 |
| antarcticus 307_gvpN | GYSVHFRGPAGTGKTTMAMHTAALLGRPVVLITGDEEMITSNLVG | |
| | AESGYNYRKVTDNYIHTVSKIEESSDRSWNDHRLTTACREGYTLIY | |
| | DEFTRSRAEANNVLLSVLEEGILVLPAQNRGEPFIKVHPNFRVIFTS | |
| | NPQEYAGVHEAQDALSDRIVTIDIGEADRELEVSIASSRSGLEVAK | |
| | TEPIVDMVRAFRDTGEYDQTPTLRACIVICRMVANEKLNTTIDDPF | |
| | FVQICLDVLGSKSTFGGKEHDKRTQQRKLLLDNLKHYCPSKVSTK | |
| | PSAKDDESKSTLIQVSSRGSL* | |
| Octadecabacter | MMPEARKDFVQTDSVKSVSRRALAYINAGYSVHFRGPAGTGKTT | 349 |
| arcticus 238_gvpN | MAMHTAALLGRPVVMITGDEEMVTSNLVGAESGYNYRKVTDNYI | |
| | HTVSKVEESSDRSWNDHRLTTACREGYTLIYDEFTRSRAEANNVL | |
| | LSVLEEGILVLPAQNRGEPFIKVHPDFRVIFTSNPQEYAGVHDAQD | |
| | ALSDRIVTIDIGAADRELEVSIASSRSGLEVAKTAPIVDMVRAFRDT | |
| | GEYDQTPTLRACIMICRMVANEKLNPTIDDSYFVQICLDVLGSKSM | |
| | FGAKEQGKRTQQEKLLLDNLSHHCPSPPPSKPSAKEAEAKPRSIQA | |
| | TSRGPA* | |
| Pelodictyon | MRRQGCDSEMNTVLEPKPMPNFVETDYIRDITSRGLTYMKAGFPV | 350 |
| phaeoclathratiforme_ | HFRGPSGTGKTTVALHLASKIGRPVVIIHGDSEYKTSDLIGSEQGYK | |
| gVPN | YRRLDDNFIHSVHKYEEDMTKQWVNNRLTIAIKKGFTLVYDEFTR | |
| | SRPEANNILLPILQEKMMSTSSSNEEEYYMKVHPEFRAIFTSNPEEY | |
| | AGVNRTQDALRDRMVTMDLDYFDYETELMITHAKSGMSLDDAE | |
| | KIVKIVRGLRESGKTEFDPTIRGSIMIAKTLNVLNARPDKTNELFKK | |
| | VCQDILTSETSRVGSKTNQERVRGIVNELIDLHS* | |
| Phormidium tenue | MNTVLQARPRNFVSTPTLERTSIRALRYLQSGYSIHLKGPAGTGKT | 351 |
| NIES-30_gvpN | TLALHLADLLARPIMLLFGDDEFKTSDLIGNQSGYTRKKVVDNYIH | |
| | SVVKVEDELRHNWTDSRLTLACREGFTMVYDEFNRSRPEVNNVL | |
| | LSALEEKLLVLPPSNNRAEYIRVSPHFRAILTSNPEEYCGVHGTQD | |
| | ALQDRLITINMPEPDELAQQQILVQKVGIDSSAALQIVQLVKAFQS | |
| | AVAPDMVSSLRPSLMIATICHDHDILPLAENADFRDVCSDILLARS | |
| | KEPAPDATRHLWNLFNRFVVSQAALVNDLSLKPEAHPTARFHGEE | |
| | EDDAPLQPLEALVESDIDDVAVEDQPVIGPQDLQGETLPEAVIPEP | |
| | QGETVVETPAEAEALPEEIARVQVSPDDIETRIFDYLDATGTASLV | |
| | NIEAALDLNRFQAVNAVKSMLDQGLIEKQETDGQLQGYQLSSN* | |
| Planktothrix agardhii | MTTVLQARPKGFVNTPTIEQLTIRALRYLQSGFSLHLRGPAGTGKT | 352 |
| str. 7805_gvpN | TLAMHLADLLNRPIVLIFGDDELKSSDLIGNQLGYTRKKVVDNFIH | |
| | SVVKLEDELRQNWIDSRLTLACKEGFTLVYDEFNRSRPEVNNVLL | |
| | SALEEKLLVLPPNNSRSEYIRVNPHFRAIFTSNPEEYCGVYGTQDAL | |
| | LDRLITIDMPEPDDETQQEILIQKIGISPEDAKNIIEIVKIYLEITTQKK | |
| | EIKPVQNGKAARPHIDKASGLRPGLIIAKICHEHDISIQENNQDFIKV | |
| | CADILLSRTNLSLTEAQNKLEKVIKTVLTDGDTSNNSFLPPSETQLT | |
| | ENNSLEIEEQVYQYLQKTTSARVSEIEVALGLNRVQTTNVLRSLLK | |
| | QGHLKQQDNRFFAVNKQGELIQP* | |
| Planktothrix | MTTVLQARPKGFVNTPTIEQLTIRALRYLQSGFSLHLRGPAGTGKT | 353 |
| rubescens_gvpN | TLAMHLADLLNRPIVLIFGDDELKSSDLIGNQLGYTRKKVIDNFIHS | |
| | VVKLEDELRQNWIDSRLTLACKEGFTLVYDEFNRSRPEVNNVLLS | |
| | ALEEKLLVLPPNNSRSEYIRVNPHFRAIFTSNPEEYCGVYGTQDAL | |
| | LDRLITIDMPEPDDETQQEILIQKIGISPEDAKNIIEIVKIYLEITTQKK | |
| | EIKPVQNGKAARPHIDKASGLRPGLIIAKICHEHDISIQENNQDFIKV | |
| | CADILLSRTNLSLTEAQNKLEKVIKTVLTDGDTSTNSFLPLSETQLT | |
| | ENNSLEIEEQVYQYLQKTTSARVSEIEVALGLNRVQTTNVLRSLLK | |
| | QGHLKQQDNRFFAVNKQGELIQP* | |
| Psychromonas | MSIENLNNVSEIKIEQSDDDHIYPEASEDFVETPYIKEVTERAMLYL | 354 |
| ingrahamii 37_gvpN1 | DAGYPVHFAGPAGTGKTTLAFHIAALRQRPVTLIHGNHEFGTSDLI | |
| | GKESGYRRHRVVDNYVHSVVKEEEELQSLWSDNRLTTCCRNGDT | |
| | LVYDEFNRSTPEANNVLLSILEEGILNLPSSRSDGYLEVHPQFRAIFT | |
| | SNPQEYAGTHATQDALVDRMITIMLHYPDRHTEVRVAIAKSGINS | |
| | DEAGSIVDIVNEFRELCGSKIVSSGPKTMPTVRASIAIARVLVQKGE | |
| | HAFRDNTFFHRICRDVLCMYTQQVSFSNRSVLDKQLEDLIMKFCP | |
| | ATYKSSGSKIRA* | |
| Psychromonas | MSINNLNISTIKIEQPENDNIYPEASAEFVQTPYIQEVTERALLYLDA | 355 |
| ingrahamii 37_gvpN2 | GYPVHFAGPAGTGKTTLAFHIAALRKRPVTLIHGNHEFGSSDLIGK | |
| | ESGYRRHRLVDNYVHSVMKEEEELKSLWVDNRLTTCCRNGDTLV | |
| | YDEFNRSTPEANNVLLSILEEGILNLPSLRSMGDGYLEVHPSFRAIF | |
| | TSNPQEYAGTHATQDALVDRMITIMLNYPDRDTEVRVAVAKSGIS | |
| | NEEAGFIVDIVNEFRELSNHKSLSSGQKSMPTVRASIAISRVLIQKG | |
| | EHAFRDNVFFHRVCHDVLCMYIQKISPSNRSFLDKQLEVLIGKFCP | |
| | AAKSALVPKVVK | |
| Rhodobacter | MTIPRDLPWGDARTPLFEDEELRSLLDRAEIYLREGIAIHFRGPAGV | 356 |
| capsulatus SB | GKTTLALHLAQRFARPVTFFVGNDWLGRADIFGRDLGETVSTVQD | |
| 1003_gvpN | HYISSVRRAERKSRIDWQEAPLARAMRDGHVLVYDEFSRSRPEAN | |
| | AALLSVIEEGVLPLSDPAAGRSHIVAHPDFRVILTSNPRDYVGVQA | |
| | VPDALLDRMITFSLDGMSFETEVGIVATAARTDPADARAICALIHL | |
| | LRAEKPGTMEISMRSGIMIARLARAAGVAPDPADPVFVQICADVL | |
| | GTRMRGSDIDDVMALLLRPDPAPAACAGGAR* | |
| Rhodobacter | MTVLSPSLPHAAGIDAALVENPWLGLRRSGRYFQNAETEALFARA | 357 |
| sphaeroides | LGYARAGVCVHLAGPAGLGKTTLALRIAQALGRPVAFMTGNEWL | |
| 2.4.1_gvpN | GSRDFIGGEIGQTVTSVVDRYIQSVRRTEQSARIDWKESILGQAMR | |
| | CGQTFIYDEFTRASPEANAALLSVLEEGVLVSTDGASRHQYIEAHP | |
| | DFRVLLTSNPHEYQGVKAAPDALIDRMVTLRLEEPSAPTLAGIVAL | |
| | RSGLDPATARRIVDLILSVQRSGEMQAPPSMRTAILVARLAAPLRL | |
| | AGRLSDAALAEIAADVLRGRGLEADAAAFEAKLAAPTPGETAR* | |
| Serratia sp. ATCC | MIKQNTVSQYTVDDDLVVPEASEHFVATSYVNDIIERALVYLRAG | 358 |
| 39006_gvpN | YPVHFAGPSGIGKTTLAFHLAALWGRPVTMLQGNEEFVSSDLTGK | |
| | DIGYRKSSLVDNYIHSVLKTEEQMNRMWVDNRLTTACRNGDMLI | |
| | YDEFNRSKAETNNVLLSVLSEGILNLPGLRGVGEGYLDVHPEFRAI | |
| | FTSNPEEYAGTHKTQDALMDRMITINIGLVDRDTELQILHARSELE | |
| | LKEAAYIVDIIRELRGNEHETKHGLRAGIAIAHILHQQGIKPRYGDK | |
| | LFHAICYDVLSMDAAKIQHAGRSIYREMVDGVIRKICPPIGSDTVK | |
| | ASTQKIKAVE* | |
| Stella | MSTEPAPVMPPSTDIEFGSQRPARPKPAEALAVGYRLSARPAAPST | 359 |
| vacuolata ATCC- | LTLRPRADFVETDQVKDLTRRGLGFLRAGYPLHFRGPAGTGKTTL | |
| 43931_gvpN | ALHVAAQLGRPVIVITGDNELGTADLVGSQRGYHYRKVVDQFIHN | |
| | VTKLEETANQRWTDHRLTTACREGYTLVYDEFTRSRPETHNVLLG | |
| | VFEEKILFLPAEAREECYIRVHPDFRAIFTSNPQEYAGVHASQDALA | |
| | DRLATIDVDYPNRAMELAVASARTGLAEAEAARIIDLVRAFRASG | |
| | DYQQTPTMRASLMIARVAAQEGLRISVDDPGFVQLCMDALESRIF | |
| | SGARQEADARARHRVALLGLLATHCPSEAPVARVATVARAKRKS | |
| | AS* | |
| Thiocapsa rosea strain | MSAKPLQDASEVSALNNDNVQPEASDTFVCTPSVEALAERASAYL | 360 |
| DSM 235 | QAGYPVHLAGPAGTGKTTLAFHAAAKRGRPVKLIHGNDELGLAD | |
| Ga0242571 11_gvpN | MVGQDNGYRRNTLVDNYIHSVVKTQEEVRTFWIDNRVTTACLNG | |
| | ETLIYDEFNRSRPEVNNIFLSILGEGILNLPNRRHQGAGYLEVHPEF | |
| | RVIFTSNPEEYAGTHKTQDALMDRMITMKIGHYDRETEIRVTRAK | |
| | SGLPPSEVAIVVDIVRELRGQSVNHHRPTLRACIAIARIMADRRISA | |
| | RSNNSFFRDICRDILDMDSAKVRRDGNALGESPVDDVVASISARAR | |
| | RPKIVEPKGLHKEI* | |
| Tolypothrix sp. PCC | MTNTENHKKRAVLRVRPGQFVVTPAIEKVAIRALRYLTSGFAIHLR | 361 |
| 7601_gvpN | GPAGTGKTTLAMHLANCLDRPIMLIFGDDEFKSSDLIGSESGYTHK | |
| | KLLDNYIHNVLKVEDELKQNWVDSRLTLACREGLTLVYDEFNRS | |
| | RPEVNNVLLSALEEKILTLPPSSNQPEYLHVHPKFRAIFTSNPEEYC | |
| | GVHSTQDALMDRLVTINMPEPDEQTQIEILTHKTGIHHEYAQLIAR | |
| | LVKAFRSATGAEKTSGLRSCLMVAKVCAEHDILVTPENTDFREICA | |
| | DVLFNRTNLSASDATTLFLELLNHVQVKPVEPVDDSDPYDVAEAE | |
| | IVGAAEPQTDAIAEPVTLDESLLSDQPN* | |
| Trichodesmium | MTTVLNVSPDRFVSTPGVERVTQRASRYLESGYSVHLRGPAGVGK | 362 |
| erythraeum | TTLALHLAHLRQQPIFLMIGDDEFKTSDLIGNKSGYTRKKLVDNYI | |
| IMS101_gvpN1 | HTVLKVEDELRDNWIDSRLTLACKEGFTLIYDEFNRSRPEVNNVLL | |
| | SVLEEKMLVLPPSQNQSEYIQVHPQFRVILTSNSEEWTGVHATQDA | |
| | LLDRVVTIGMEQPDISTEQNIVIQKTGINPLKAEVIIKLVRSVRQRV | |
| | DKEDLGSLRSALMISKVCHDHDIPLDGKDSSFSDLCADILISRPNLP | |
| | RQEALQQLDEVLEEFFPADQPSSSDVGLEKEGSL* | |
| Trichodesmium | MTTVLNVSPDRFVSTPSVERVTQRASRYLESGYSVHLRGPAGVGK | 363 |
| erythraeum | TTLALHLAHLRQQPIFLMIGDDEFKTSDLIGNKSGYTRKKLVDNYI | |
| IMS101_gvpN2 | HTVLKVEDELKHNWIDSRLTLACKEGFTLIYDEFNRSRPEVNNVLL | |
| | SVLEEKMLVLPPSQNQSEYIQVHPQFRVILTSNSEEWTGVHATQDA | |
| | LLDRVVTIGMGQPDISTEQNIIIQKTGINPLKAEVIIKLVRSVRERLE | |
| | TEDLGSLRSALMISKVCHDHDIPLGGKDSNFSDLCADILISRANLPR | |
| | QEALKQLDEVLEELFPADQLSISDIGLKKEGSL* | |
| gvpV | | |
| Anabaena-flos- | MIKNIQVFFMKTISNRSISRAKISTMPRPKSDASSQLDLYKMVTEK | 364 |
| aquae_gvpV | QRIQRDMYSIKERMGLLQQRLDILNQQIEATEKTIHKLRQPHSNTA | |
| | QNIVRSNIFVESNNYQTFEVEY* | |
| Aphanizomenon flos- | MKSFRHRSIIRAKISTMPRHISEASSQLELYKMVAEKQRISRELSSIK | 365 |
| aquae NIES-81_gvpV | ERMATLQKRLDSLNNEIDNTEKTIHKLRQPHSSTAQNIVRSKNVVE | |
| | SNNYQTFEIEY* | |
| Arthrospira platensis | MRYKYHRQIQPKLSAIPRQKSQANLYRNSYLLAVEKKRLTEELEV | 473 |
| NIES-39_gvpV | LQSRSHIIEQRLALIEDQLGELEKDVTQLSVPPSPKPQNNLPVNNPE | |
| | PPPQSNPTNSSHINTFMVDY* | |
| Burkholderia | MPIPKKGLHDIRFRHAPGATPLPVHSMYMRISCIEMEKSRRTIERRA | 366 |
| thailandensis sp. | AQRRIAAVDSRVADLEREKARLYAAIDNEAPQAGDIRGSFRIRY* | |
| Bp5365 strain | | |
| MSMIB43_gvpV | | |
| Desulfobacterium | MLKNRNRSIKGVQNIKTHAGKVDHVSHPHMAYMRISCLEMEKAR | 367 |
| vacuolatum DSM | KNKEKSGAQKRIDMINQRLMEIEKEKAHIQRILGDTSIALESSNVD | |
| 3385_gvpV | HDSEIKGGFKIKY* | |
| Desulfomonile tiedjei | MNIRMKGNSRGLRDIRTHSGKVDRVGLPYMAYMSISCLEMEKAR | 368 |
| DSM 6799_gvpV | REKERLSALTRIKNIEQRIREIEAEKDLLLKGVGERTRTDLQKASTP | |
| | RDQSAQCKGGFKIRY* | |
| Legionella drancourtii | MMPALVKGLRNIKTMSNRLDKVQSPHEAFISAAALHREKQRHLQ | 369 |
| LLAP12_gvpV | ELAILRNRLDEINLRLEQINEQQNQVAEAFDISPPRAVKSALRTGIQ | |
| | SKTGSTSHGFKIKY* | |
| Microcystis aeruginosa | MTTTRPPRPIRSKISTMPRKQSEADHQLELYKLITEKQRIQEKLEM | 370 |
| NIES-843_gvpV | MERQIQQLKNRLTFVTEQIETTEQSIQNLRTANPPSVAKKPDSPKT | |
| | VAHSSNNSSNFQTFYLEY* | |
| Nostoc punctiforme | MHRTPNRRQIQAKLSTMPPQRSQATVYLNAYKMMLEKERLEEEL | 371 |
| ATCC 29133_gvpV | EKLEARRHQIQQRLAILNSQTIPEENMTHQQANTDLENNTPKFNTL | |
| | TLEY* | |
| Nostoc sp. PCC | MLSIIQVFPMTKVRNRGIIRPKITTMPRNKSEASSQLELYKLVTEQQ | 372 |
| 7120_gvpV | RIKQELAFIEQRTVLLKQRLSTLKTQIEGTERSINHLRHSELKYSRIA | |
| | LPKIFSETNNYQAFDIEY* | |
| Planktothrix agardhii | MRPFRSQPPILPKISTMPRQKTEATLYRSLYQLAVEKKRLQEELESL | 373 |
| str. 7805_gvpV | GQRFETVTQRLQQIETQIQGLETDVKQIAPPKPPETKPNQPSTPTPT | |
| | KAEPGSVSTFTLDY* | |
| Psychromonas | MTAAKRKTLRGLADIRTISSCGTSGQEAYQMYLKRGVLEMEKLR | 374 |
| ingrahamii 37_gvpV1 | RQKEKNSALERVTNINRRLMAIDTDIDFLCQSLKVIEKRTNQENSIV | |
| | EKSVSRGFKLRY* | |
| Psychromonas | MIFSKKKNALRGLADIRTLSGCGTSGQEAYQMYLKRGVLEMEKL | 375 |
| ingrahamii 37_gvpV2 | RRQKEKNSALERVRNINYRLMAIDADIDFLCQSLKVIEERTNKENS | |
| | ISNESVTYKKGFKLRY* | |
| Serratia sp. ATCC | MAISTRPLRTLSDIKTHSGRVSGEHQTYRDYFQIGALELERWRRTR | 376 |
| 39006_gvpV | EREAASSRIASIDERIADIDKEKAALLADATAASAVAENNDKSEAA | |
| | EKKKKSSGLRIKY* | |
| Thiocapsa rosea strain | MSKFTQPSRSVRDIKTLAGMADDVRAPHKMYMRLFALETERHRR | 377 |
| DSM 235 | LQERASAMLRVDNIDARCAEIAEEMEQLLQILGVEAVAPGGPPAN | |
| Ga0242571_11_gvpV | ARPGSGRVPTQPHRGRGKGTGAGRQTTSGETSVGEAVKIRY* | |
| gvpW | | |
| Anabaena-flos- | MELENLYTYAFLEIPSSPLILPQGAANQVVLINGTELAAIVEPGIFLE | 378 |
| aquae_gvpW | SFQNNDEKIIQMALSHDRVICELFQQITVLPLRFGTYFTSTNNLLNH | |
| | LKSHEKEYQNKLEKINGKNEFTLKLIPRMIEEIVPSEGGGKDYFLA | |
| | KKQRYQNQNNFSIAQAAEKQNLIDLITKVNQLPVVVQEQEEQIQIY | |
| | LLVSCQDKTLLLEQFLTWQKACPRWDLLLGDCLPPYHFI* | |
| Aphanizomenon flos- | MELENLYTYAFLKTPSFSLHLPQGSTTSVIQIDGNGLSAIVEPGISLD | 379 |
| aquae NIES-81_gvpW | SFQDDDEKIVQMAIEHDRVICDIFRQITVLPLRFGTYFANTDNLLTH | |
| | LESYGQEYLDKLEKINCKTEFILKLIPRMITEESPVLESGRHYFLAK | |
| | KQHYQRQKNFILAQASEKEILINFISKINQIPVIIQEQEEEVRIYLLVN | |
| | YQDKTLLLEQFLTWQQTCPRWDLFLGEGIPPYHFI* | |
| Arthrospira platensis | MYVYAFIKSQSISWKSVQGIYEPVVLLEAGALAAVVEPNLQAENL | 380 |
| NIES-39_gvpW | SADNEEELMRAVLTHDRIVCQIFEETTVLPVRFGTCFDSEARLCEH | |
| | LTTEGDRYFRQLEKLTGRAEYLLEAIPQPFNQEKPSSDTTAPPTKG | |
| | RDYFLQKKRLHQQRLNFEQQQEQQWQDFINAIASKYPIVQGKATE | |
| | DAERIYLLIPRSQEVALVEWVAQQQQNIDLWEFSLGNAVPAYHFL | |
| | * | |
| Dolichospermum | MKLENFYTYAFLEIPRFPLVLPQGAASQVILINGSGMSAIVEPGISLE | 381 |
| circinale_gvpW | SFQNNDEKIIQMALSHDRVICELFQQVTVLPLRFGTCFTSTNNLLN | |
| | YLELHRQEYQEKLEKINGKIEFTLKLIPQTMEEPAPLERGGRDYFL | |
| | AKKQRYQDQNNFRIAQAAEKQNLIDSISKVNQLPFVIQEKEEEVNI | |
| | YLLVKSEDKTLLLEQFLNWQKACPRWDLLLGEPLPPYHFI | |
| Microcystis aeruginosa | MKLYNLYTYAFLKTPIESLKLPVGMANPLLLITGGELSAVVEPEVG | 382 |
| NIES-843_gvpW | LDTLQNDDERLIQSVLCHDRVICQLFQQTTILPLRFGTSFLEAENLL | |
| | THLCSHGQEYQEKIEELEGKGEYLLKCIPRKPEEPVLFSESKGRQYF | |
| | LAKKQLYEAQQDFYTLQGSEWQNLVNLITQSYPSTRIITAPGTESRI | |
| | YLLVNLQEEPLLIEQVLHWQKACPRWELQLGQVSPPYHFT* | |
| Nostoc punctiforme | MSIYAYALLVPTASPLVLPLGMERNTELVYSSGLAALVEPEISLEAI | 383 |
| ATCC 29133_gvpW | QATDERLLQAVLNHDHVIRELFQQTPLLPLRFGRGFTSVEKLLNHL | |
| | ENHQEQYLETLTQLADKVEYSVKVTACSLLDDSDTIDARGKAYLL | |
| | AKKQRYQTQQAFQAQQCEQWELLNELILKTYTNVICETRQSDVR | |
| | QIHFLAQRNDSTLSTQLFSLWQVQCSHWQLALSEPLPPYHFLKNTL | |
| | I* | |
| Nostoc sp. PCC | MRSPNFYTYAFLNTPDIPLRLPSGNLGQLLLIHGHKLSAVVEPGISL | 384 |
| 7120_gvpW | ESSQNNDEEVIKMVLAHDRVICELSQQTTVLPLRFGTYFNSEETLL | |
| | NHIESHAQEYQKKLDHIQGKTEYTLKLIPRKFEELAKVSGGNGRD | |
| | YFLAKKLHYEHQKNFIGDQNREKNHLINLIMDVYRSSAIIQDYVEE | |
| | VRLHLLVDRHDKTLLFKQVLTLQEKCPHWNLILGEPLPPYHFV* | |
| gvpR | | |
| Bacillus- | MEIKKIMQAVNDFFGEHVAPPHKITSVEATEDEGWRVIVEVIEERE | 385 |
| megaterium_gvpR | YMKKYAKDEMLGTYECFVNKEKEVISFKRLDVRYRSAIGIEA* | |
| gvpS | | |
| Bacillus- | MSLKQSMENKDIALIDILDVILDKGVAIKGDLIISIAGVDLVYLDLR | 386 |
| megaterium_gvpS | VLISSVETLVQAKEGNHKPITSEQFDKQKEELMDATGQPSKWTNP | |
| | LGS* | |
| Rhodococcus hoagii | MSATPDRRIALVDLLDRVLGGGVVVAGEITLSIADVDMVHISLRTL | 387 |
| 103S_gvpS | VSSVSALTRPPDEKPENDG* | |
| gvpT | | |
| Bacillus- | MATETKLDNTQAENKENKNAENGSKEKNGSKASKTTSSGPIKRA | 388 |
| megaterium_gvpT | VAGGIIGATIGYVSTPENRKSLLDRIDTDELKSKASDLGTKVKEKS | |
| | KSSVASLKTSAGSLFKKDKDKSKDDEENVNSSSSETEDDNVQEYD | |
| | ELKEENQTLQDRLSQLEEKMNMLVELSLNKNQDEEAEDTDSDEEE | |
| | NDENDENDENEQDDENEEETSKPRKKDKKEAEEEESESDEDSEEE | |
| | EEDSRSNKKNKKVKTEEEDEDESEEEKKEAKPKKSTAKKSKNTKA | |
| | KKNTDEEDDEATSLSSEDDTTA* | |
| gvpU | | |
| Bacillus- | MSTGPSFSTKDNTLEYFVKASNKHGFSLDISLNVNGAVISGTMISA | 389 |
| megaterium_gvpU | KEYFDYLSETFEEGSEVAQALSEQFSLASEASESNGEAEAHFIHLK | |
| | NTKIYCGDSKSTPSKGKIFWRGKIAEVDGFFLGKISDAKSTSKKSS* | |

TABLE 7
Protein sequences of gvpC from exemplary species:

| | UniProt | | SEQ |
|---|---|---|---|
| Species | ID No. | Amino acid Sequence | ID NO: |
| Anabaena | P09413 | MISLMAKIRQEHQSIAEKVAELSLETREFLSVTTAKRQEQAEK | 390 |
| flos-aquae | | QAQELQAFYKDLQETSQQFLSETAQARIAQAEKQAQELLAFH | |
| | | KELQETSQQFLSATAQARIAQAEKQAQELLAFYQEVRETSQQ | |
| | | FLSATAQARIAQAEKQAQELLAFHKELQETSQQFLSATADAR | |
| | | TAQAKEQKESLLKFRQDLFVSIFG | |
| Halobacterium | P24574 | MSVTDKRDEMSTARDKFAESQQEFESYADEFAADITAKQDD | 391 |
| salinarum | | VSDLVDAITDFQAEMTNTTDAFHTYGDEFAAEVDHLRADID | |
| | | AQRDVIREMQDAFEAYADIFATDIADKQDIGNLLAAIEALRTE | |
| | | MNSTHGAFEAYADDFAADVAALRDISDLVAAIDDFQEEFIAV | |
| | | QDAFDNYAGDFDAEIDQLHAAIADQHDSFDATADAFAEYRD | |
| | | EFYRIEVEALLEAINDFQQDIGDFRAEFETTEDAFVAFARDFY | |
| | | GHEITAEEGAAEAEAEPVEADADVEAEAEVSPDEAGGESAGT | |
| | | EEEETEPAEVETAAPEVEGSPADTADEAEDTEAEEETEEEAPE | |
| | | DMVQCRVCGEYYQAITEPHLQTHDMTIQEYRDEYGEDVPLR | |
| | | PDDKT | |
| Halobacterium | Q02228 | MSVKDKREKMTATREEFAEVQQAFAAYADEFAADVDDKRD | 392 |
| mediterranei | | VSELVDGIDTLRTEMNSTNDAFRAYSEEFAADVEHFHTSVAD | |
| | | RRDAFDAYADIFATDVAEMQDVSDLLAAIDDLRAEMDETHE | |
| | | AFDAYADAFVTDVATLRDVSDLLTAISELQSEFVSVQGEFNG | |
| | | YASEFGADIDQFHAVVAEKRDGHKDVADAFLQYREEFHGVE | |
| | | VQSLLDNIAAFQREMGDYRKAFETTEEAFASFARDFYGQGA | |
| | | APMATPLNNAAETAVTGTETEVDIPPIEDSVEPDGEDEDSKAD | |
| | | DVEAEAEVETVEMEFGAEMDTEADEDVQSESVREDDQFLDD | |
| | | ETPEDMVQCLVCGEYYQAITEPHLQTHDMTIKKYREEYGED | |
| | | VPLRPDDKA | |
| Microchaete | P08041 | MTPLMIRIRQEHRGIAEEVTQLFKDTQEFLSVTTAQRQAQAK | 393 |
| diplosiphon | | EQAENLHQFHKDLEKDTEEFLTDTAKERMAKAKQQAEDLFQ | |
| | | FHKEMAENTQEFLSETAKERMAQAQEQARQLREFHQNLEQT | |
| | | TNEFLADTAKERMAQAQEQKQQLHQFRQDLFASIFGTF | |
| Nostoc sp. | Q8YUS9 | MTALMVRIRQEHRSIAEEVTQLFRETHEFLSATTAHRQEQAK | 394 |
| | | QQAQQLHQFHQNLEQTTHEFLTETTTQRVAQAEAQANFLHK | |
| | | FHQNLEQTTQEFLAETAKNRTEQAKAQSQYLQQFRKDLFASI | |
| | | FGTF | |

TABLE 8
Amino acid sequences of exemplary GVS and GVA proteins from B. megaterium.

| GVA | | SEQ ID |
|---|---|---|
| Protein | Amino acid sequence | NO.: |
| gvpB | MSIQKSTNSSSLAEVIDRILDKGIVIDAFARVSVVGIEILTIEARVVIASVDTWL | 395 |
| | RYAEAVGLLRDDVEENGLPERSNSSEGQPRFSI | |
| gvpR | MEIKKIMQAVNDFFGEHVAPPHKITSVEATEDEGWRVIVEVIEEREYMKKYAKD | 396 |
| | EMLGTYECFVNKEKEVISFKRLDVRYRSAIGIEA | |
| gvpN | MTVLTDKRKKGSGAFIQDDETKEVLSRALSYLKSGYSIHFTGPAGGGKTSLARA | 397 |
| | LAKKRKRPVMLMHGNHELNNKDLIGDFTGYTSKKVIDQYVRSVYKKDEQVSE | |
| | NWQDGRLLEAVKNGYTLIYDEFTRSKPATNNIFLSILEEGVLPLYGVKMTDPFVR | |
| | VHPDFRVIFTSNPAEYAGVYDTQDALLDRLITMFIDYKDIDRETAILTEKTDVEE | |
| | DEARTIVTLVANVRNRSGDENSSGLSLRASLMIATLATQQDIPIDGSDEDFQTLCI | |
| | DILHHPLTKCLDEENAKSKAEKIILEECKNIDTEEK | |
| gvpF | MSETNETGIYIFSAIQTDKDEEFGAVEVEGTKAETFLIRYKDAAMVAAEVPMKIY | 398 |
| | HPNRQNLLMHQNAVAAIMDKNDTVIPISFGNVFKSKEDVKVLLENLYPQFEKLF | |
| | PAIKGKIEVGLKVIGKKEWLEKKVNENPELEKVSASVKGKSEAAGYYERIQLGG | |
| | MAQKMFTSLQKEVKTDVFSPLEEAAEAAKANEPTGETMLLNASFLINREDEAKF | |
| | DEKVNEAHENWKDKADFHYSGPWPAYNFVNIRLKVEEK | |
| gvpG | MLHKLVTAPINLVVKIGEKVQEEADKQLYDLPTIQQKLIQLQMMFELGEIPEEAF | 399 |
| | QEKEDELLMRYEIAKRREIEQWEELTQKRNEES | |
| gvpL | MGELLYLYGLIPTKEAAAIEPFPSYKGFDGEHSLYPIAFDQVTAVVSKLDADTYS | 400 |
| | EKVIQEKMEQDMSWLQEKAFHHHETVAALYEEFTIIPLKFCTIYKGEESLQAAIEI | |
| | NKEKIENSLTLLQGNEEWNVKIYCDDTELKKGISETNESVKAKKQEISHLSPGRQ | |
| | FFEKKKIDQLIEKELELHKNKVCEEIHDKLKELSLYDSVKKNWSKDVTGAAEQM | |
| | AWNSVFLLPSLQITKFVNEIEELQQRLENKGWKFEVTGPWPPYHFSSFA | |
| gvpS | MSLKQSMENKDIALIDILDVILDKGVAIKGDLIISIAGVDLVYLDLRVLISSVETLV | 401 |
| | QAKEGNHKPITSEQFDKQKEELMDATGQPSKWTNPLGS | |
| gvpK | MQPVSQANGRIHLDPDQAEQGLAQLVMTVIELLRQIVERHAMRRVEGGTLTDE | 402 |
| | QIENLGIALMNLEEKMDELKEVFGLDAEDLNIDLGPLGSLL | |
| gvpJ | MAVEHNMQSSTIVDVLEKILDKGVVIAGDITVGIADVELLTIKIRLIVASVDKAKE | 403 |
| | IGMDWWENDPYLSSKGANNKALEEENKMLHERLKTLEEKIETKR | |
| gvpT | MATETKLDNTQAENKENKNAENGSKEKNGSKASKTTSSGPIKRAVAGGIIGATI | 404 |
| | GYVSTPENRKSLLDRIDTDELKSKASDLGTKVKEKSKSSVASLKTSAGSLFKKDK | |
| | DKSKDDEENVNSSSSETEDDNVQEYDELKEENQTLQDRLSQLEEKMNMLVELS | |
| | LNKNQDEEAEDTDSDEEENDENDENDENEQDDENEEETSKPRKKDKKEAEEEE | |
| | SESDEDSEEEEEDSRSNKKNKKVKTEEEDEDESEEEKKEAKPKKSTAKKSKNTK | |
| | AKKNTDEEDDEATSLSSEDDTTA | |
| gvpU | MSTGPSFSTKDNTLEYFVKASNKHGFSLDISLNVNGAVISGTMISAKEYFDYLSE | 405 |
| | TFEEGSEVAQALSEQFSLASEASESNGEAEAHFIHLKNTKIYCGDSKSTPSKGKIF | |
| | WRGKIAEVDGFFLGKISDAKSTSKKSS | |

TABLE 9
Amino acid sequences of exemplary GVS and GVA proteins from Serratia sp..

| GVA | | SEQ ID |
|---|---|---|
| Protein | Amino acid sequence | NO.: |
| gvpA1 | MAKVOKSTDSSSLAEVVDRILDKGIVIDAWVKVSLVGIELLSIEARVVIASVETY | 406 |
| | LKYAEAIGLTASAATPA | |
| gvpA2 | MPVNKQYQDEQQQVSLCEALDRVLNKGVVIVADITISVANIDLIYLSLQALVSSV | 407 |
| | EAKNRLPGRE | |
| gvpA3 | MPVNKQYQDEQQQVSLCEALDRVLNKGVVIVADITISVANIDLIYLSLQALVSSV | 408 |
| | EAKNRLPGRE | |
| gvpC | MGCLTDGMAQLRKNIDDSHESRIAQQNARVSSVSAQIAGFSTTRARNAAQDAR | 409 |
| | ARATFVADNVRGVNRMLSDFCHTREVMSRQQSEERATFVTDMSKKTLALLDGF | |
| | NAERKSMAERCAKERADFIANVANDVAAFLSASEKDRMAAHAVFFGMTLAKK | |
| | KTSLAV | |
| gvpN | MIKQNTVSQYTVDDDLVVPEASEHFVATSYVNDIIERALVYLRAGYPVHFAGPS | 410 |
| | GIGKTTLAFHLAALWGRPVTMLQGNEEFVSSDLTGKDIGYRKSSLVDNYIHSVL | |
| | KTEEQMNRMWVDNRLTTACRNGDMLIYDEFNRSKAETNNVLLSVLSEGILNLP | |
| | GLRGVGEGYLDVHPEFRAIFTSNPEEYAGTHKTQDALMDRMITINIGLVDRDTEL | |
| | QILHARSELELKEAAYIVDIIRELRGNEHETKHGLRAGIAIAHILHQQGIKPRYGD | |
| | KLFHAICYDVLSMDAAKIQHAGRSIYREMVDGVIRKICPPIGSDTVKASTQKIKA | |
| | VE | |
| gvpV | MAISTRPLRTLSDIKTHSGRVSGEHQTYRDYFQIGALELERWRRTREREAASSRI | 411 |
| | ASIDERIADIDKEKAALLADATAASAVAENNDKSEAAEKKKKSSGLRIKY | |
| gvpF1 | MMSIDKSRNHRAKVLYALCVSDDSTPNYKIRGLEAAPVYSIDQDGLRAVVSDTL | 412 |
| | STRLRPERRNITAHQAVLHKLTEEGTVLPMRFGVIARNAEAVKNLLVANQDTIR | |
| | EHFERLDGCVEMGLRVSWDVTNIYEYFVATYPVLSETRDEIWNGNSNANNHRE | |
| | EKIRLGNLYESLRSGDRKESTEKVKEVLLDYCEEIIENPVKKEKDVMNLACLVA | |
| | RERMDEFAKGVFEASKLFDNVYLFDYTGPWAPHNFVTLDLHAPTAKKKTLTRA | |
| | GTLSD | |
| gvpF2 | MTMNTEAQTEQAIYLYGLTLPDLAAPPILGVDNQHPINTHQCAGLNAVISPVALS | 413 |
| | DFTGEKGEDNVQNVTWLTPRICRHAQIIDSLMAQGPVYPLPFGTLFSSQNALEQE | |
| | MKSRATDVFVSLRRITGCQEWALEATLDRKQAVDVLFTEGLDSGRFCLPEAIGR | |
| | RHLEEQKLRRRLTTELSDWLAHALTAMQNELHPLVRDFRSRRLLDDKILHWAY | |
| | LLPVEDVAAFQQQVADIVERYEAYGFSFRVTGPWAAYSFCQPDES | |
| gvpF3 | MSLLLYGIVAEDTQLALEPDGSPHAGEEPMQLVKAATLAALVKPCEADVSREPA | 414 |
| | AALAFGQQIMHVHQQTTIIPIRYGCVLADEDAVTQHLLNHEAHYQTQLVELENC | |
| | DEMGIRLSLASAEDNAVTTPQASGLDYLRSRKLAYAVPEHAERQAALLNNAFT | |
| | GLYRRHCAEISMFNGQRTYLLSYLVPRTGLQAFRDQFNTLANNMTDIGVISGPW | |
| | PPYNFAS | |
| gvpG | MLLIDDILFSPVKGVMWIFRQIHELAEDELAGEADRIRESLTDLYMLLETGQITED | 415 |
| | EFEQQEAVLLDRLDALDEEDDMLGDEPGDDEDDEYEEDDDEEDDDEEDDDDE | |
| | DDDDEDDDDEEDDDDDEDDDDEDEPEGTTK | |
| gvpW | MKPAIYPKFLLESPLKLVFFGGKGGVGKSTCATSTALRLAQEQPQHHFLLVSTDP | 416 |
| | AHSLQNILSDLVLPKNLDVRELNAAASLHEFKSQHEGVLKEIAYRGTVLDQNDV | |
| | QGLMDTALPGMDELAAYLEIAEWIQKDTYYRIIIDTAPTGHTLRLLEMPDLIYRW | |
| | LTALDTLLAKQRYIRKRFAGDNRLDHLDHFLLDMNDSLKAMHELVTDSTRCCF | |
| | VLVMLAEAMSVEESIDLAGALNQQRVFLSDLVVNRLFPENDCPTCCVERNRQM | |
| | LALQNGYQRLPGHVFWTLPLLAIEPRGALLHEFWSGVRLLDENEVMATTCHHQ | |
| | LPLRVESSISLPASTFRLLIFAGKGGVGKTTLACATALRLNSEYPELRILLFSADPA | |
| | HSLSDCLGVTLQQQPISVLVNIDAQEINAQADFDKIRQGYRAELEAFLLDTLPNL | |
| | DITFDREVLEHLLDLAPPGLDEIMALTAIMDHLDSGRYDMVIVDGAPSGHLLRLL | |
| | ELPELIRDWLKQFFSLLLKYRKVMRFPHLSERLVQLSRELKNLRALLQDTKQTG | |
| | LYAVTVPTHLALEKTYEMTCALQRLGLTANALFINQITPPSDCTLCQAITSRESLE | |
| | LKCADEMFPSQPHAQIFRQTEPTGLSKLKTLGSALFL | |
| gvpK | MTTNQLSHHSPVFGPTSPAIQRPITEANRHKIDIDGERVRDGLAQLVLTLVKLLH | 417 |
| | ELLERQAIRRMDSGSLSDEEVERLGLALMRQAEELTHLCDVFGFKDDDLNLDLG | |
| | PLGRLL | |
| gvpX | MVNTTNDINAATRGLLLRMGNAWFEQDELRQAVDIYLKIIEQYPDSKESKTAQT | 418 |
| | ALLTISQRYERDGLFRLSLDILERVGEITPTSI | |
| gvpY | MRALIHFPIIHSPKDLGTLSEAASHLRTETQTRAYLAAVEGFWTMITTTIEGLDLD | 419 |
| | YTHLKLYQDGLPVCGKENEIVTDVANAGSQNYKLLLTLQHKGAILMGTESPELL | |
| | LQERDLMTQLLQSTEQTEASLETAKTLLNRRDDYIAQRIDETLQDGEMAILFLGL | |
| | MHNIEAKLPADIVFIQPLGKPPGGESI | |
| gvpH | MTGNVEGILRGLGDLVEKLVETGEQIKRSGAFDIDTNDGKNAKAVYGFSIKMGL | 420 |
| | DGNQENRVEPFGNIRRDEQTGEATVQEVSEPLVDVIEESDHVLVLAEMPGVADE | |
| | DVQVELNGDILTLHSERGSKKYHKEIVLPCSFDDKAMERSCRNGILEVKLGK | |
| gvpZ | MSEELKLKVAEALPKDAGRGYARLDPADMARLNLAVGDIVQLTSKKGTGIAKL | 421 |
| | MPTYPDMRNKGIVQLDGLTRRNTSLSLDEKVQIEPASCKHATQIVLIPTTITPNQR | |
| | DLDYIGSLLDGLPVQKGDLLRAHLFGSRSADFKVESTIPDGAVLIDPTTTLVIGKS | |
| | NAVGNSSHSTQRLSYEDVGGLKNQVRRIREMIELPLRYPEVFERLGIDAPKGVLL | |
| | SGPPGCGKTLIARIIAQETDAQFFTISGPEIVHKFYGESEAHLRKIFEEAGRKGPSII | |
| | FLDEIDSIAPHRDKVVGDVEKRIVAQLLALMDGLKNRGKVIVIAATNLPNAIDPA | |
| | LRRPGRFDREISIPIPDREGRREIIEIHSTGMPLNADVDLNVLADITHGFVGADLEA | |
| | LCREAAMSALRRLLPEIDFSSAELPYDRLAELTVMMDDFRAALCEVSPSAIRELF | |
| | VDIPDVRWEDVGGLDDVRRRLIESVEWPIKYPELYEQAGVKPPKGLLLAGPPGV | |
| | GKTLIAKAVANESGVNVISVKGPALMSRYVGDSEKGVRELFLKARQAAPCIIFLD | |
| | EVDSVIPARNEGAIDSHVAERVLSQFLSEMDGLEELKGVFVMGATNRADLIDPA | |
| | MLRPGRFDEIIELGLPDEDARRQILAVHLRNKPLGDNIHADDLAERCDGASGAEL | |
| | AAVCNRAALAALRRAIQQSEEAVLSPSTVGETPVALTVRIEQHDFAEVIAEMFG | |
| | DDA | |

TABLE 10
Amino Acid Sequences of GV proteins from Anabaena flos-aquae

| | | SEQ |
|---|---|---|
| gvp | | ID |
| gene | Sequence | NO: |
| gvpA | MAVEKTNSSSSLAEVIDRILDKGIVIDAWVRVSLVGIELLAIEARIVIASVETYLK | 422 |
| | YAEAVGLTQSAAVPA | |
| gvpC | MISLMAKIRQEHQSIAEKVAELSLETREFLSVTTAKRQEQAEKQAQELQAFYKD | 423 |
| | LQETSQQFLSETAQARIAQAEKQAQELLAFHKELQETSQQFLSATAQARIAQAEK | |
| | QAQELLAFYQEVRETSQQFLSATAQARIAQAEKQAQELLAFHKELQETSQQFLS | |
| | ATADARTAQAKEQKESLLKFRQDLFVSIFG | |
| gvpN | MTTTKVNHKRAVLRLRPGQFVVTPAIERVAIRALRYLKSGFPVHLRGPAGTGKT | 424 |
| | TLAMHLANCLDRPVMLLFGDDQFKSSDLIGSESGYTHKKVLDNYIHSVVKLEDE | |
| | FKQNWVDSRLTLACREGFTLVYDEFNRSRPEVNNVLLSALEEKILSLPPSSNQPE | |
| | YLSVNPQFRVIFTSNPEEYAGVHSTQDALMDRLVTISMPEPDEITQTEILIQKTNID | |
| | RESANFIVRLVKSFRLATGAEKTSGLRSCLMIAKVCADNNIPVTTESLDFPDIAIDI | |
| | LFNRSHLSMSESTNIFLELLDKFSAEELEILNNRVTGDNDFLIDNSQFVSQQLAGQ | |
| | PN | |
| gvpJ | MLPTRPQTNSSRTINTSTQGSTLADILERVLDKGIVIAGDISISIASTELVHIRIRLLI | 425 |
| | SSVDKAKEMGINWWESDPYLSTKAQRLVEENQQLQHRLESLEAKLNSLTSSSVK | |
| | EEIPLAADVKDDLYQTSAKIPSPVDTPIEVLDFQAQSSGGTPPYVNTSMEILDFQA | |
| | QTSAESSSPVGSTVEILDFQAQTSEESSSPVVSTVEILDFQAQTSEESSSPVGSTVEI | |
| | LDFQAQTSEEIPSSVDPAIDV | |
| gvpK | MVCTPAENFNNSLTIASKPKNEAGLAPLLLTVLELVRQLMEAQVIRRMEEDLLS | 426 |
| | EPDLERAADSLQKLEEQILHLCEMFEVDPADLNINLGEIGTLLPSSGSYYPGQPSS | |
| | RPSVLELLDRLLNTGIVVDGEIDLGIAQIDLIHAKLRLVLTSKPI | |
| gvpF | MSIPLYLYGIFPNTIPETLELEGLDKQPVHSQVVDEFCFLYSEARQEKYLASRRNL | 427 |
| | LTHEKVLEQTMHAGFRVLLPLRFGLVVKDWETIMSQLINPHKDQLNQLFQKLA | |
| | GKREVSIKIFWDAKAELQTMMESHQDLKQQRDNMEGKKLSMEEVIQIGQLIEIN | |
| | LLARKQAVIEVFSQELNPFAQEIVVSDPMTEEMIYNAAFLIPWESESEFSERVEVI | |
| | DQKFGDRLRIRYNNFTAPYTFAQLDS | |
| gvpG | MLTKLLLLPIMGPLNGVVWIAEQIQERTNTEFDAQENLHKQLLSLQLSFDIGEIGE | 428 |
| | EEFEIQEEEILLKIQALEEEARLELEAEQEEARLELEAEQEDFEYPPQFTAEVNKD | |
| | QHLVLLP | |
| gvpV | MIKNIQVFFMKTISNRSISRAKISTMPRPKSDASSQLDLYKMVTEKQRIQRDMYSI | 429 |
| | KERMGLLQQRLDILNQQIEATEKTIHKLRQPHSNTAQNIVRSNIFVESNNYQTFE | |
| | VEY | |
| gvpW | MELENLYTYAFLEIPSSPLILPQGAANQVVLINGTELAAIVEPGIFLESFQNNDEKII | 430 |
| | QMALSHDRVICELFQQITVLPLRFGTYFTSTNNLLNHLKSHEKEYQNKLEKINGK | |
| | NEFTLKLIPRMIEEIVPSEGGGKDYFLAKKQRYQNQNNFSIAQAAEKQNLIDLITK | |
| | VNQLPVVVQEQEEQIQIYLLVSCQDKTLLLEQFLTWQKACPKWDLLLGDCLPPY | |
| | HFI | |

### Example 5: Identification of Alternative B. megaterium Gene Cluster Detectable by TEM in E. coli

The Gas Vesicle gene cluster of Table 8 above was tested to identify possible alternative clusters detectable by TEM.

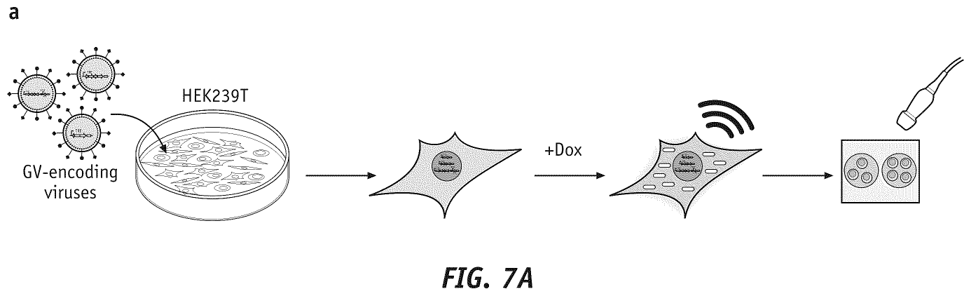

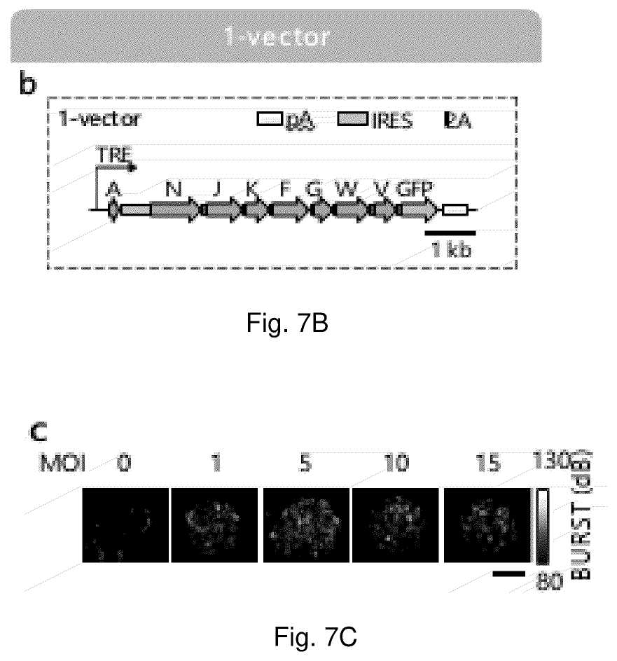

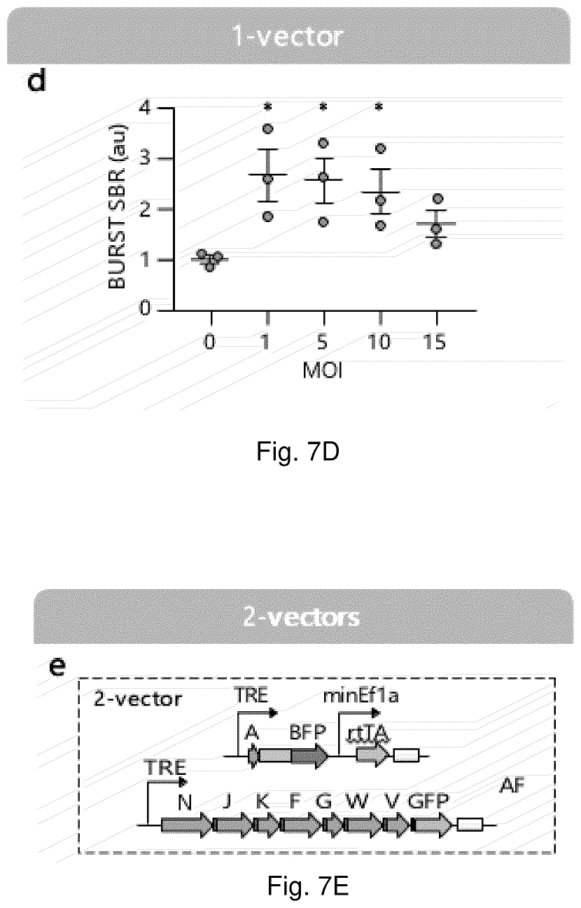

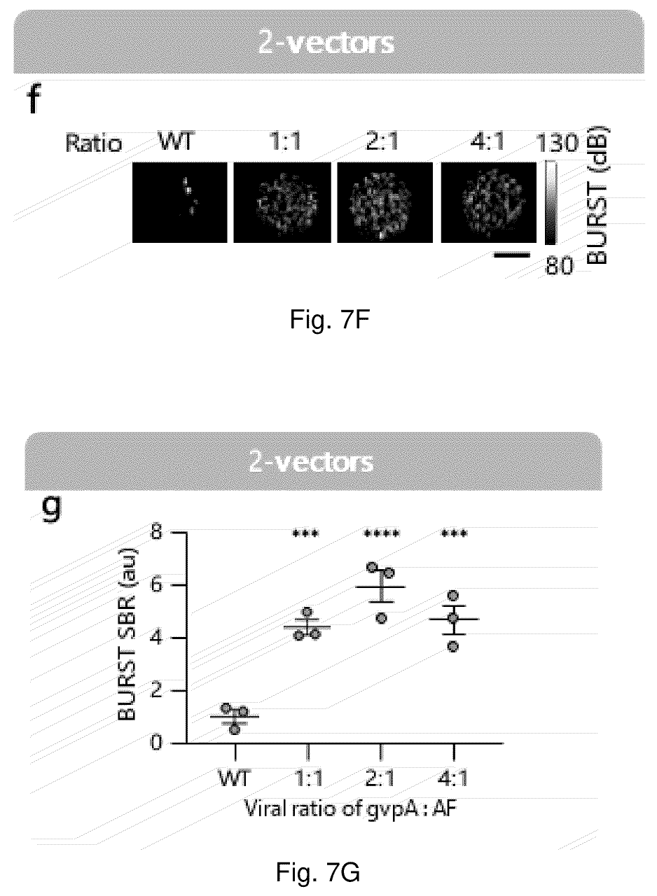

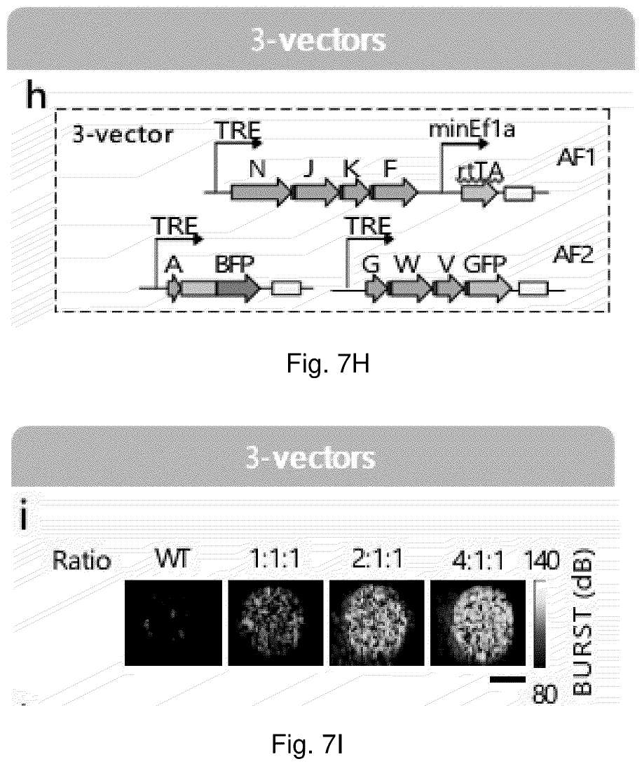

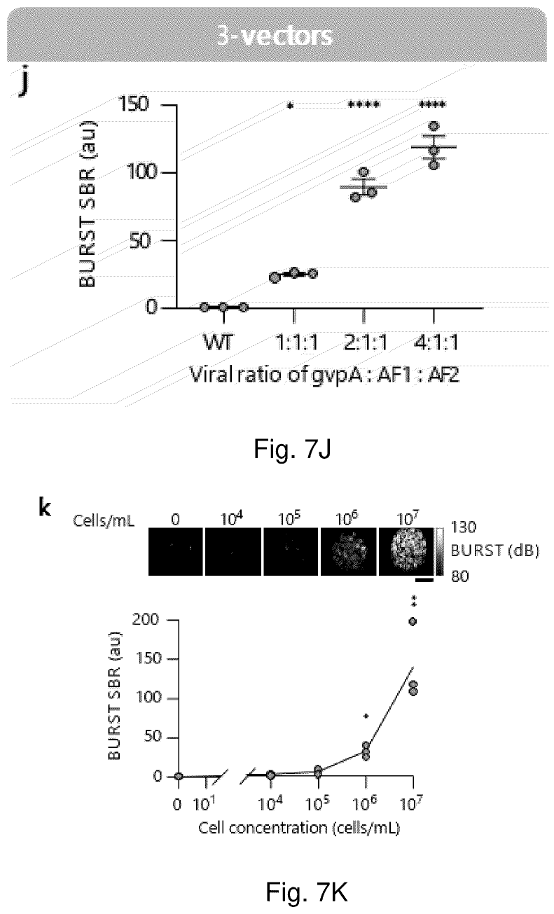

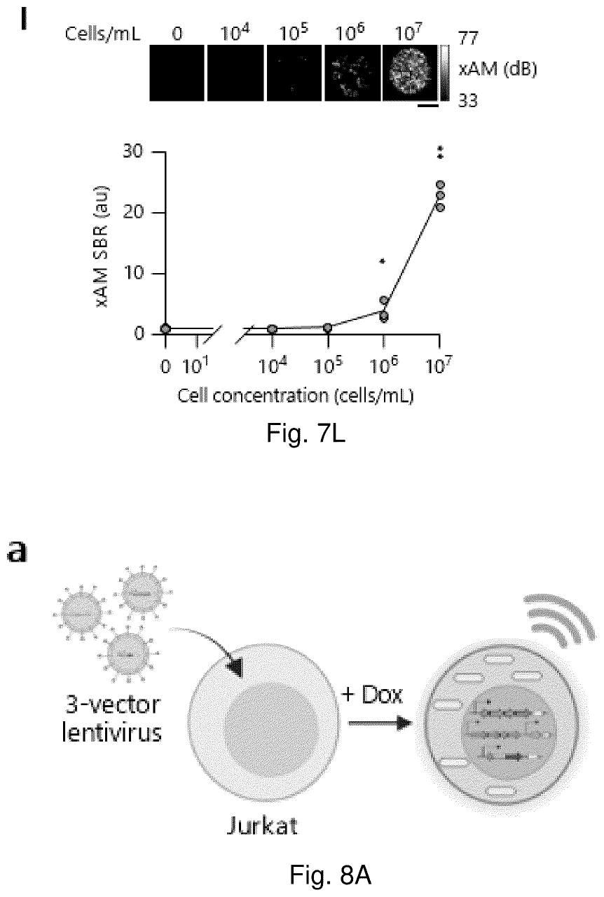

In particular, the B. megaterium gene cluster can be expressed in E. coli Rosetta 2(DE3)pLysS cells using the two construct schematically illustrated in FIG. 7 top panel.

The formation of gas vesicles was detected through Transmission Electronic Microscopy (TEM) after expression of gas vesicles genes for 22 hours.

The results shown in FIG. 7 bottom panel indicate that gvpR and gvpT genes in the B. megaterium gene cluster are not necessary for gas vesicle formation.

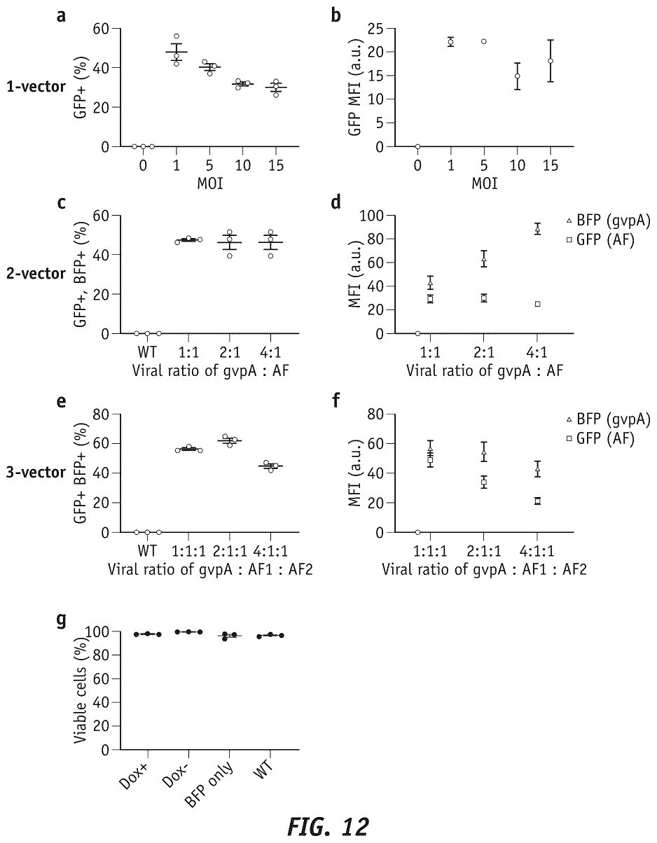

Therefore, the following alternative GV cluster including 9 gvp gene sequences of B. megaterium genes shown in the following Table 11 and FIG. 12B was identified as detectable by TEM and ultrasound in mammalian cells (HEK293 and CHO-K1).

TABLE 11
Gvp genes of exemplary GV gene cluster from B. megaterium

| | | Seq ID |
|---|---|---|
| Gene | Sequence | NO: |
| gvpB | ATGAGCATCCAGAAGTCCACCAACAGCAGCAGCCTGGCCGAAGTGAT | 431 |
| | CGACCGGATCCTGGACAAGGGCATCGTGATCGACGCCTTCGCCAGAG | |
| | TGTCCGTCGTGGGCATCGAGATCCTGACCATCGAGGCCAGAGTCGTG | |
| | ATCGCCAGCGTGGACACCTGGCTGAGATATGCCGAAGCCGTGGGCCT | |
| | GCTGCGGGACGACGTGGAAGAAAATGGCCTGCCCGAGCGGAGCAAC | |
| | AGCTCTGAGGGACAGCCCCGGTTCAGCATCTGA | |
| gvpN | ATGACCGTGCTGACCGACAAGCGGAAGAAGGGCAGCGGCGCCTTCAT | 432 |
| | CCAGGACGACGAGACAAAAGAGGTGCTGAGCAGAGCCCTGAGCTAC | |
| | CTGAAGTCCGGCTACAGCATCCACTTCACCGGACCTGCCGGCGGAGG | |
| | CAAGACATCTCTGGCTAGAGCCCTGGCCAAGAAACGGAAGCGGCCCG | |
| | TGATGCTGATGCACGGCAACCACGAGCTGAACAACAAGGACCTGATC | |
| | GGCGATTTCACCGGCTACACCAGCAAGAAAGTGATCGACCAGTACGT | |
| | GCGGAGCGTGTACAAGAAAGACGAACAGGTGTCCGAGAACTGGCAG | |
| | GACGGCAGACTGCTGGAAGCCGTGAAGAATGGCTACACCCTGATCTA | |
| | CGACGAGTTCACCAGAAGCAAGCCCGCTACCAACAACATCTTCCTGA | |
| | GCATCCTGGAAGAGGGCGTGCTGCCCCTGTACGGCGTGAAGATGACC | |
| | GACCCTTTCGTGCGCGTGCACCCCGACTTCAGAGTGATCTTCACCAGC | |
| | AACCCCGCCGAGTATGCCGGCGTGTACGATACCCAGGACGCCCTGCT | |
| | GGACCGGCTGATCACCATGTTCATCGACTACAAGGACATCGACCGGG | |
| | AAACCGCCATCCTGACCGAGAAAACCGACGTGGAAGAGGACGAGGC | |
| | CCGGACCATCGTGACCCTGGTGGCCAACGTGCGGAACAGAAGCGGCG | |
| | ACGAGAATAGCAGCGGCCTGAGCCTGAGAGCCAGCCTGATGATTGCC | |
| | ACCCTGGCCACCCAGCAGGACATCCCTATCGATGGCAGCGACGAGGA | |
| | CTTCCAGACCCTGTGCATCGACATCCTGCACCACCCCCTGACCAAGTG | |
| | CCTGGACGAGGAAAACGCCAAGAGCAAGGCCGAGAAGATCATTCTG | |
| | GAAGAGTGCAAGAACATCGACACCGAGGAAAAGTGA | |
| gvpF | ATGAGCGAGACAAACGAGACAGGCATCTACATCTTCAGCGCCATCCA | 433 |
| | GACCGACAAGGACGAGGAATTCGGCGCCGTGGAAGTGGAAGGGACC | |
| | AAGGCCGAGACATTCCTGATCCGGTACAAGGACGCCGCCATGGTGGC | |
| | CGCCGAAGTGCCCATGAAGATCTACCACCCCAACCGGCAGAACCTGC | |
| | TGATGCACCAGAATGCCGTGGCCGCCATCATGGACAAGAACGACACC | |
| | GTGATCCCCATCAGCTTCGGCAACGTGTTCAAGAGCAAAGAGGACGT | |
| | GAAGGTGCTGCTGGAAAACCTGTACCCCCAGTTCGAGAAGCTGTTCC | |
| | CCGCCATCAAGGGAAAGATCGAAGTGGGCCTGAAAGTGATCGGCAA | |
| | GAAAGAGTGGCTGGAAAAGAAAGTGAACGAGAACCCCGAGCTGGAA | |
| | AAAGTGTCCGCCAGCGTGAAGGGCAAGAGCGAGGCCGCTGGCTACT | |
| | ACGAGAGAATCCAGCTGGGCGGCATGGCCCAGAAGATGTTCACCAGC | |
| | CTGCAGAAAGAAGTGAAAACCGACGTGTTCAGCCCCCTGGAAGAAG | |
| | CCGCCGAGGCCGCCAAAGCCAATGAGCCTACAGGCGAGACAATGCT | |
| | GCTGAACGCCAGCTTCCTGATCAACAGAGAGGACGAGGCCAAGTTCG | |
| | ACGAAAAAGTGAATGAGGCCCACGAGAACTGGAAGGATAAGGCCGA | |
| | CTTCCACTACAGCGGCCCCTGGCCCGCCTACAACTTCGTGAACATCCG | |
| | GCTGAAGGTGGAAGAGAAGTGA | |
| gvpG | ATGCTGCACAAGCTCGTGACCGCCCCCATCAACCTGGTCGTGAAGAT | 434 |
| | CGGCGAGAAGGTGCAGGAAGAGGCCGACAAGCAGCTGTACGACCTG | |
| | CCCACCATCCAGCAGAAGCTGATCCAGCTGCAGATGATGTTCGAGCT | |
| | GGGCGAGATCCCCGAGGAAGCCTTCCAGGAAAAAGAGGACGAGCTG | |
| | CTGATGAGATACGAGATCGCCAAGCGGCGCGAGATCGAGCAGTGGG | |
| | AGGAACTGACCCAGAAGCGGAACGAGGAAAGCTGA | |
| gvpL | ATGGGCGAGCTGCTGTACCTGTACGGCCTGATCCCCACCAAAGAGGC | 435 |
| | CGCTGCCATCGAGCCCTTCCCATTCTACAAGGGCTTCGACGGCGAGC | |
| | ACAGCCTGTACCCTATCGCCTTCGACCAAGTGACCGCCGTGGTGTTCA | |
| | AGCTGGACGCCGACACCTACAGCGAGAAAGTGATCCAGGAAAAGAT | |
| | GGAACAGGACATGAGCTGGCTGCAGGAAAAGGCCTTCCACCACCAC | |
| | GAGACAGTGGCCGCCCTGTACGAGGAATTCACCATCATCCCCCTGAA | |
| | GTTCTGCACCATCTATAAGGGCGAGGAATCCCTGCAGGCCGCCATCG | |
| | AGATCAACAAAGAGAAGATCGAGAACTCCCTGACCCTGCTGCAGGGC | |
| | AACGAGGAATGGAACGTGAAGATCTACTGCGACGACACCGAGCTGA | |
| | AGAAGGGCATCAGCGAGACAAACGAGAGCGTGAAGGCCAAGAAGCA | |
| | GGAAATCAGCCACCTGAGCCCCGGCAGACAGTTCTTCGAGAAGAAGA | |
| | AGATTGACCAGCTGATCGAGAAAGAGCTGGAACTGCACAAGAACAA | |
| | AGTGTGCGAGGAAATCCACGACAAGCTGATTGAGCTGAGCCTGTACG | |
| | ACTCCGTGAAGAAGAACTGGTCCAAGGACGTGACCGGCGCTGCCGAA | |
| | CAGATGGCCTGGAACAGCGTGTTCCTGCTGCCCAGCCTGCAGATCAC | |
| | CAAGTTCGTGAACGAGATCGAGGAACTGCAGCAGCGGCTGGAAAAC | |
| | AAGGGCTGGAAGTTCGAAGTGACCGGCCCCTGGCCTCCCTACCACTT | |
| | CAGCAGCTTTGCCTGA | |
| gvpS | ATGAGCCTGAAGCAGAGCATGGAAAACAAGGATATCGCCCTGATCG | 436 |
| | ACATCCTGGACGTGATCCTGGACAAGGGCGTGGCCATCAAGGGCGAC | |
| | CTGATCATCTCTATCGCCGGCGTGGACCTGGTGTACCTGGACCTGAGA | |
| | GTGCTGATCTCCAGCGTGGAAACCCTGGTGCAGGCCAAAGAGGGCAA | |
| | CCACAAGCCCATCACCAGCGAGCAGTTCGACAAGCAGAAAGAGGAA | |
| | CTGATGGACGCCACCGGCCAGCCCAGCAAGTGGACAAATCCTCTGGG | |
| | CAGC | |
| gvpK | ATGCAGCCCGTGTCCCAGGCCAACGGCAGAATCCACCTGGATCCCGA | 437 |
| | TCAGGCCGAACAGGGACTGGCCCAGCTCGTGATGACCGTGATCGAGC | |
| | TGCTGCGGCAGATCGTGGAACGGCACGCCATGAGAAGAGTGGAAGG | |
| | CGGCACCCTGACCGACGAGCAGATCGAGAATCTGGGAATCGCCCTGA | |
| | TGAACCTGGAAGAGAAGATGGACGAGCTGAAAGAGGTGTTCGGACT | |
| | GGACGCCGAGGACCTGAACATCGACCTGGGCCCTCTGGGCAGCCTGC | |
| | TGTGA | |
| gvpJ | ATGGCCGTGGAACACAACATGCAGAGCAGCACCATCGTGGACGTGCT | 438 |
| | GGAAAAGATCCTGGACAAGGGCGTCGTGATCGCCGGGGACATCACA | |
| | GTGGGAATCGCCGACGTGGAACTGCTGACCATCAAGATCCGGCTGAT | |
| | CGTGGCCAGCGTGGACAAGGCCAAAGAAATCGGCATGGATTGGTGG | |
| | GAGAACGACCCCTACCTGAGCAGCAAGGGCGCCAACAACAAGGCCC | |
| | TGGAAGAGGAAAACAAGATGCTGCACGAGCGGCTGAAAACACTGGA | |
| | AGAGAAGATCGAGACAAAGCGCTGA | |
| gvpU | ATGAGCACCGGCCCCAGCTTCAGCACCAAGGACAACACCCTGGAATA | 439 |
| | CTTCGTGAAGGCCAGCAACAAGCACGGCTTCAGCCTGGACATCAGCC | |
| | TGAACGTGAACGGGGCCGTGATCAGCGGCACCATGATCAGCGCCAAA | |
| | GAGTACTTCGACTACCTGAGCGAGACATTCGAAGAGGGCAGCGAGGT | |
| | GGCCCAGGCCCTGTCTGAGCAGTTTAGCCTGGCCAGCGAGGCCTCCG | |
| | AGTCTAATGGCGAAGCCGAGGCCCACTTCATCCACCTGAAGAACACC | |
| | AAGATCTACTGCGGCGACAGCAAGAGCACCCCCAGCAAGGGCAAGA | |
| | TCTTCTGGCGCGGCAAGATCGCCGAGGTGGACGGATTCTTCCTGGGA | |
| | AAGATCAGCGACGCCAAGTCCACCAGCAAGAAGTCCAGCTGA | |

Each gene is cloned in pCMVSport plasmid which contains CMV promoter upstream of each gene and SV40 polyadenylation tail downstream of each gene, as illustrated in FIG. 12B. The gene cassettes elements of the pCMVSport plasmid are reported in Table 11a below.

TABLE 11a
Additional elements of the GVP cassettes

| | | SEQ |
|---|---|---|
| | | ID |
| Element | Sequence | NO: |
| CMV | CGTTACATAACTTACGGTAAATGGCCCGCCTGGCTGACCGCCCAACGAC | 440 |
| enhancer/ | CCCCGCCCATTGACGTCAATAATGACGTATGTTCCCATAGTAACGCCAA | |
| CMV | TAGGGACTTTCCATTGACGTCAATGGGTGGAGTATTTACGGTAAACTGC | |
| promoter | CCACTTGGCAGTACATCAAGTGTATCATATGCCAAGTACGCCCCCTATT | |
| | GACGTCAATGACGGTAAATGGCCCGCCTGGCATTATGCCCAGTACATGA | |
| | CCTTATGGGACTTTCCTACTTGGCAGTACATCTACGTATTAGTCATCGCT | |
| | ATTACCATGGTGATGCGGTTTTGGCAGTACATCAATGGGCGTGGATAGC | |
| | GGTTTGACTCACGGGGATTTCCAAGTCTCCACCCCATTGACGTCAATGG | |
| | GAGTTTGTTTTGGCACCAAAATCAACGGGACTTTCCAAAATGTCGTAAC | |
| | AACTCCGCCCCATTGACGCAAATGGGCGGTAGGCGTGTACGGTGGGAG | |
| | GTCTATATAAGCAGAGCT | |
| SV40 | AACTTGTTTATTGCAGCTTATAATGGTTACAAATAAAGCAATAGCATCAC | 441 |
| polyadenylation | AAATTTCACAAATAAAGCATTTTTTTCACTGCATTCTAGTTGTGGTTTGTC | |
| tail | CAAACTCATCAATGTATCTTATCATGTCTGGATC | |

### Example 6: Rational Partitioning of a Natural Gas Vesicle Gene Cluster (GVGC) into a Multi-Cassette System of the Disclosure

The purpose of this example is to describe the rational design process for converting a large, natural Gas Vesicle Gene Cluster (GVGC) into a multi-cassette expression system. Natural GVGCs, which often exist as single, large polycistronic operons in their native organisms, are not optimized for balanced, high-yield expression in heterologous hosts, such as mammalian cells.

This procedure outlines how a comparative analysis of diverse, natural GVGCs can be used to identify conserved, separable functional modules. This modular logic is then applied to partition a “full” GV gene cluster (e.g., from Anabaena flos-aquae) into distinct genetic cassettes, enabling precise stoichiometric control of GV assembly.

The design process involves two main stages: (1) analysis of natural GVGC architectures to identify functional modules, and (2) partitioning a selected source cluster based on this modular logic.

With respect to the comparative analysis of natural GVGCs, a panel of representative GVGCs from diverse domains (Archaea, Cyanobacteria, Firmicutes, etc.) was compiled and analyzed, as shown in Table 12 below.

TABLE 12
Comparative Analysis of natural GVGCs

| | | | Presence | Presence | |
|---|---|---|---|---|---|
| | | | of AF1 | of AF2 | |
| Organism/ | | Representative | (gvpN, gvpJ, | (gvpG, gvpW, | |
| System | Domain | gvp Genes | gvpK, gvpF) | gvpV) | Notes |
| Halobacterium | Archaea | gvpA, C, N, J, | Present | gvpG | Canonical |
| salinarum (and | | K, L, M, O, F, | | present; | archaeal gvp |
| other | | G | | gvpV/W | system; |
| haloarchaea) | | | | absent | produces rigid, |
| | | | | | spindle-shaped |
| | | | | | vesicles. |
| Methanococcus | Archaea | gvpA, C, N, J, | Present | gvpG | Similar to |
| vannielii | | K, L, F, G | | present; | haloarchaea; |
| | | | | gvpV/W | supports |
| | | | | absent | ancient lineage |
| | | | | | of AF1 core. |
| Anabaena flos- | Cyanobacteria | gvpA, C, N, J, | Present | gvpG, | Classic |
| aquae | | K, F, G, V, W | | gvpV, | cyanobacterial |
| | | | | gvpW | “full” cluster; V |
| | | | | present | and W encode |
| | | | | | late-stage |
| | | | | | structural |
| | | | | | helpers. |
| Microcystis | Cyanobacteria | gvpA, C, N, J, | Present | gvpG, | Nearly identical |
| aeruginosa | | K, F, G, V, W | | gvpV, | to Anabaena |
| | | | | gvpW | cluster. |
| | | | | present | |
| Planktothrix | Cyanobacteria | gvpA, C, N, J, | Present | gvpG, | Same |
| rubescens | | K, F, G, V, W | | gvpV, | architecture as |
| | | | | gvpW | Anabaena, used |
| | | | | present | for buoyancy |
| | | | | | regulation. |
| Bacillus | Firmicutes | gvpA, C, N, J, | Present | gvpG | Bacterial but |
| megaterium | | K, F, G | | present; | lacks V/W, |
| | | | | gvpV/W | confirming |
| | | | | absent | their optional |
| | | | | | status. |
| Serratia sp. | Proteobacteria | gvpA, C, N, J, | Present | gvpG | Facultative |
| ATCC 39006 | | K, F, G | | present; | aquatic |
| | | | | gvpV/W | bacterium; |
| | | | | absent | cluster |
| | | | | | homologous to |
| | | | | | Bacillus. |
| Arthrospira | Cyanobacteria | gvpA, C, N, J, | Present | gvpG, | Retains full |
| platensis | | K, F, G, V, W | | gvpV, | cyanobacterial |
| (Spirulina) | | | | gvpW | gene module. |
| | | | | present | |

The goal of the analysis was to identify which genes are universally conserved versus which are accessory or domain-specific.

The Identification of Functional Modules can be performed, and the analysis in Table 3 reveals a clear modular structure. First, a Structural Protein, the gvpA gene, is universally present and encodes the primary structural protein. Second, a Core Assembly (AF1) module, comprising a core set of assembly factors, gvpN, gvpJ, gvpK, and gvpF, is also universally conserved across all known gvp clusters, suggesting they form an essential, core assembly machine (termed AF1).

Third, an Accessory Assembly (AF2) module exists, as the remaining genes are more variable. While gvpG is broadly conserved, gvpV and gvpW are notably present in Cyanobacteria (like Anabaena or Microcystis) while being absent in Archaea (like Halobacterium) and Firmicutes (like Bacillus). This suggests gvpG, gvpW, and gvpV form a distinct “accessory” module (termed AF2) that may assist in late-stage assembly or vesicle maturation, especially in cyanobacterial hosts.

Based on this modular logic, rational partitioning was performed, and the “full” Anabaena flos-aquae cluster (which contains all identified genes) was selected as the source. It was partitioned into three distinct cassettes, each corresponding to a primary functional module.

The comparative analysis of natural GVGCs (Table 12) provides the direct rationale for the engineered multi-cassette configuration (Table 13). In particular the analysis summarized informed the partitioning of the Anabaena flos-aquae gene cluster into the three-cassette system detailed in Table zzz.

TABLE 13
Engineered multi-cassette configuration

| | | | Presence | Presence | |
|---|---|---|---|---|---|
| | Domain | | of AF1 | of AF2 | |
| Organism/ | (Gene | Representative | (gvpN, J, | (gvpG, gvpW, | |
| System | Source) | gvp Genes | K, F) | gvp V) | Notes |
| GVGC | Cyanobacteria | gvpA | Absent | Absent | Encodes the |
| Cassette 1 | (A. flos-aquae) | | | | primary |
| (Structural) | | | | | structural |
| | | | | | protein. |
| | | | | | Separated for |
| | | | | | high-level |
| | | | | | stoichiometric |
| | | | | | control. |
| GVGC | Cyanobacteria | gvpN, gvpJ, | Present | Absent | Contains the |
| Cassette 2 | (A. flos-aquae) | gvpK, gvpF | | | universally |
| (AF1 | | | | | conserved “core” |
| Module) | | | | | assembly factors. |
| | | | | | This module is |
| | | | | | identified as |
| | | | | | AF1. |
| GVGC | Cyanobacteria | gvpG, gvpW, | Absent | Present | Contains the |
| Cassette 3 | (A. Eflos-aquae) | gvpV | | | remaining |
| (AF2 | | | | | assembly factors, |
| Module) | | | | | including the |
| | | | | | “accessory” |
| | | | | | proteins gvpV/W. |

This example illustrates the transition from a natural, single-operon GVGC to a rationally designed multi-cassette system. The comparative analysis (Table 12) confirmed that the gvp gene cluster is not a monolithic block, but rather a composite of functional modules (Structural, Core Assembly, and Accessory Assembly) that have been mixed and matched during evolution.

By partitioning the “full” Anabaena cluster according to this observed modularity (Table 13), the resulting system is no longer constrained by the fixed, natural stoichiometry of a single operon. This 3-cassette design is the key architectural feature that enables independent tuning of the gene dosage for each functional module, allowing for the optimization of GV yield in a heterologous host.

### Example 7: Configuration of a Multi-Cassette Gas Vesicle Genetic Circuit (GVGC) Based on Conserved Gene Modules

The purpose of this procedure is to rationally design a multi-cassette Gas Vesicle Genetic Circuit (GVGC) by partitioning a full gene cluster based on the comparative analysis of conserved gene modules. This example uses the gene cluster from Anabaena flos-aquae, which is known to contain the full set of genes identified in the cyanobacterial domain. The design objective is to separate the primary structural protein (gvpA) from the core assembly factors (AF1) and the accessory assembly factors (AF2) to enable stoichiometric tuning.

An exemplified procedure comprises selecting a source organism, identifying and partitioning gene modules to provide a 3 cassette GVGC system which can be stoichiometrically configured in accordance with the disclosure.

In an exemplary realization Anabaena flos-aquae was selected as the source of the gvp genes. This organism is classified as a Cyanobacterium containing the “full” gene cluster, including gvpA, the AF1 module (gvpN, J, K, F), and the complete AF2 module (gvpG, V, W).

Based on conserved modules identified in a comparative table, the 8 essential genes from the Anabaena cluster (gvpA, N, J, K, F, G, W, V) can then be partitioned into three distinct functional cassettes.

Cassette 1 (Structural): gvpA. This gene encodes the main structural protein and is separated to allow its expression level to be independently modulated³.

Cassette 2 (AF1 Module): gvpN, gvpJ, gvpK, gvpF. This cassette contains the “core” AF1 module, which is universally conserved across all gvp gene clusters (as shown in the comparative table).

Cassette 3 (AF2 Module): gvpG, gvpW, gvpV. This cassette contains the remaining assembly factors, including gvpG and the cyanobacteria-specific “helper” proteins gvpV and gvpW.

This partition results in a 3-cassette GVGC design, where each cassette can be delivered on a separate vector to control the input gene dosage, as detailed in Table 14.

TABLE 14

| | | | Presence | Presence | |
|---|---|---|---|---|---|
| | Domain | | of AF1 | of AF2 | |
| Organism/ | (Gene | Representative | (gvpN, J, | (gvpG, gvpW, | |
| System | Source) | gvp Genes | K, F) | gvp V) | Notes |
| GVGC | Cyanobacteria | gvpA | Absent | Absent | Encodes the |
| Cassette 1 | (A. flos-aquae) | | | | primary structural |
| (Structural) | | | | | protein. Separated |
| | | | | | for high-level |
| | | | | | stoichiometric |
| | | | | | control. |
| GVGC | Cyanobacteria | gvpN, gvpJ, | Present | Absent | Contains the |
| Cassette 2 | (A. flos-aquae) | gvpK, gvpF | | | universally |
| (AF1 | | | | | conserved “core” |
| Module) | | | | | assembly factors. |
| | | | | | This module is |
| | | | | | identified as AF1 |
| | | | | | in the |
| | | | | | comparative |
| | | | | | analysis. |
| GVGC | Cyanobacteria | gvpG, gvpW, | Absent | Present | Contains the |
| Cassette 3 | (A. flos-aquae) | gvpV | | | remaining |
| (AF2 | | | | | assembly factors, |
| Module) | | | | | including the |
| | | | | | “accessory” |
| | | | | | proteins gvpV/W |
| | | | | | specific to the |
| | | | | | cyanobacterial |
| | | | | | cluster. |

### Example 8: Quantification of Stoichiometric Expression Ratios (Dosage Index) for a Multi-Cassette System

The purpose of this procedure is to determine the intrinsic expression strength, or Dosage Index (DI), for the individual genetic cassettes of a multi-construct Gas Vesicle (GV) expression system. This experiment quantifies the relative expression levels encoded by the specific promoters and regulatory regions for the gvpA, AF1 (e.g., gvpNJKF), and AF2 (e.g., gvpGWV) cassettes when delivered at an identical, single-copy gene dosage.

The resulting DI ratio (DI_{gupA}:DI_{AF1}:DI_{AF2}) is used to verify that the system's genetic design achieves the target stoichiometric balance required for optimal GV production, such as a ratio of at least 2:1:1.

To ensure an equal input gene dosage, corresponding to a 1:1:1 genomic copy number ratio, three distinct stable mammalian cell lines can be generated, for example, in a HEK293T parent line.

Each cell line contained a single, stably integrated copy of one of the cassettes at the same genomic locus. Line A (gvpA) contained the gvpA cassette linked to a BFP reporter. Line B (AF1) contained the AF1 cassette linked to a distinct reporter, such as Reporter X or mCherry. Line C (AF2/Reference) contained the AF2 cassette linked to a GFP reporter, and this Line C was designated as the internal reference for all DI calculations. All three stable cell lines (Line A, B, and C), along with a negative control parent cell line, were then cultured under identical Standard Conditions. This included induction with doxycycline (1 uM/mL) for 72 hours to drive expression from the promoters. Following the 72-hour induction, cells from all lines were harvested. The expression output for each cassette was quantified by measuring the Mean Fluorescence Intensity (MFI) per cell via flow cytometry.

This experimental approach and resulting analysis are summarized in Table 15.

TABLE 15
Dosage Index (DI) Calculation and Analysis

| Analysis Step | Approach/Data | Conclusion/Discussion |
|---|---|---|
| 1. Reference | The AF2-GFP cassette (Line C) | Its DI is set to 1.0 by definition. All |
| Selection | was selected as the internal | other cassettes are measured relative |
| | reference. | to this unit. |
| 2. Output | MFI was measured for each cell | The raw data shows gvpA has the |
| Measurement | line after 72 h induction: gvpA | strongest expression, followed by |
| (Hypothetical | (BFP): 12,000 MFI units AF1 | AF2, and then AF1. |
| Data) | (Reporter X): 2,500 MFI units | |
| | AF2 (GFP - Ref): 3,000 MFI | |
| | units | |
| 3. DI Calculation | DI = (Output_{Cassette})/ | The calculated DI values quantify |
| | (Output_{Reference}) | the intrinsic relative expression |
| | DI_gvpA: 12,000/3,000 = 4.0 | strength encoded by each cassette's |
| | DI_AF1: 2,500/3,000 = 0.83 | regulatory elements. |
| | DI_AF2: 3,000/3,000 = 1.0 | |
| 4. Final Ratio | The resulting ratio is: | This ratio meets the target |
| Determination | DI_{gvpA}:DI_{AF1}:DI_{AF2} | stoichiometric balance. The gvpA |
| | 4.0:0.83:1.0 | component (4.0) is within the |
| | | desired range of 2.0 to 4.0 relative |
| | | to the assembly factors. |
| | | Furthermore, the AF1 component |
| | | (0.83) is approximately equal to the |
| | | AF2 component (1.0). |

Based on the quantitative analysis of expression from single-copy integrants, the genetic architecture of this system produces a Dosage Index ratio of 4.0:0.83:1.0 DI ratio (DI_{gpA}:DI_{AF1}:DI_{AF2}). This intrinsic stoichiometric ratio, achieved under identical gene dosage, fulfills the design objective of a ratio of at least 2:1:1. This quantitative verification confirms the system's design provides the targeted stoichiometric imbalance, with a high relative expression of the gvpA structural protein, which is correlated with enhanced functional GV yield.

### Example 9: Engineering Stoichiometric Ratios Via Regulatory Element Selection (Promoter Tuning)

The purpose of this procedure is to rationally design a multi-cassette system that intrinsically encodes a desired Dosage Index (DI) ratio by selecting regulatory elements, specifically promoters, of different, characterized strengths. This method aims to achieve a target DI ratio, for example DI_{gvpA}:DI_{AF1}:DI_{AF2}≈4:1:1, by hard-coding the relative expression strengths into the genetic cassettes themselves.

The objective is to design a 3-cassette system that intrinsically produces gvpA, AF1, and AF2 proteins at an approximate 4:1:1 molar ratio when delivered at an equal gene dosage. First, the AF2 cassette was designated as the reference module. A well-characterized, moderate-strength constitutive promoter, such as the human Elongation Factor 1 alpha (hEF1a) promoter, was selected to drive its expression; by definition, this cassette's intrinsic strength is set as DI_{AF2}=1.0. For the cassette design, to achieve a ratio of approximately 1.0 relative to the reference, the same hEF1a promoter was selected to drive the AF1 cassette, resulting in an expected DI_{AF1}≈1.0. To achieve a ratio of approximately 4.0, a strong viral promoter, such as the SFFV promoter (known to have ≈4-5 fold higher activity than hEF1a in many cell types), was selected to drive the gvpA cassette, resulting in an expected DI_{gvpA}≈4.0. For verification, the three distinct cassettes were constructed. To confirm the engineered DI ratio, the procedure from Example 8 is followed: each cassette is stably integrated as a single copy into the same genomic locus in three separate cell lines. The relative expression output is measured under Standard Conditions to confirm the target DI_{gvpA}:DI_{AF1}:DI_{AF2} ratio of 4:1:1.

The corresponding results and Analysis are reported in Table 16

TABLE 16
Engineering DI ratio promoter tuning

| Target | Selected Regulatory | Expected Relative | Resulting |
|---|---|---|---|
| Module | Element (Promoter) | Strength | Encoded DI |
| gvpA | SFFV (Strong | ≈4 × | 4.0 |
| (Structural) | Viral) | Reference | |
| AF1 (Core | hEF1a (Moderate | ≈1 × | 1.0 |
| Assembly) | Constitutive) | Reference | |
| AF2 | hEF1a (Reference) | 1 × | 1.0 (Reference) |
| (Accessory | | Reference | |
| Assembly) | | | |

This procedure illustrates how the target DI ratio (DI_{gvpA}:DI_{AF1}:DI_{AF2}) can be achieved by encoding the relative stoichiometry directly into the DNA of the cassettes using promoters of different strengths. This “DI-encoded” system is robust, as the desired 4:1:1 expression ratio is intrinsically established by the constructs themselves. This approach is advantageous when an equal gene dosage (e.g., 1:1:1 viral ratio or co-integration) is desired or easier to achieve, as the promoter selection dictates the final functional output.

### Example 10: Engineering Stoichiometric Ratios Via Gene Dosage (Copy Number) Control

The purpose of this procedure is to achieve a target functional protein ratio (e.g., 4:1:1) by manipulating the input gene dosage or copy number of the delivered cassettes. This approach is distinct from Example 9; it assumes all cassettes are built with identical regulatory elements (and thus have an intrinsic DI ratio of 1:1:1) and achieves the final stoichiometric imbalance by delivering different amounts of each genetic construct.

The objective is to produce gvpA, AF1, and AF2 proteins at a 4:1:1 molar ratio by delivering three separate lentiviral vectors at a non-equal ratio. For the cassette design, three separate lentiviral transfer plasmids were constructed. To isolate the effect of gene dosage, all three cassettes (gvpA, AF1, AF2) were placed under the control of identical regulatory elements, such as a TRE3G promoter and a WPRE. Therefore, the intrinsic DI ratio of the constructs themselves is DI_{gvpA}:DI_{AF1}:DI_{AF2}=1:1:1. For delivery and dosing, high-titer lentivirus was produced for all three constructs. The functional titer was precisely measured for each viral stock. Target mammalian cells (e.g., HEK293T) were then co-transduced with the three viruses at a viral ratio (Multiplicity of Infection, MOI) of 4:1:1. For instance, to infect 10⁶ cells, a total of 6 MOI might be used, composed of a gypA virus at MOI=4, an AF1 virus at MOI=1, and an AF2 virus at MOI=1. Finally, for analysis, the resulting functional ultrasound signal was measured. This signal is the output of the final, functional 4:1:1 protein ratio, which was achieved by delivering a 4:1:1 ratio of the 1:1:1 DI constructs.

The corresponding results and Analysis are reported in Table 17

TABLE 17
Engineering DI ratio gene dosage

| | Intrinsic DI of | Input Gene | | Resulting |
|---|---|---|---|---|
| | Construct (from | Dosage (Viral | | Functional |
| Target Module | identical promoters) | Ratio/MOI) | | Protein Ratio |
| gvpA | 1.0 | 4 | parts | 4.0 |
| (Structural) | | | | |
| AF1 (Core | 1.0 | 1 | part | 1.0 |
| Assembly) | | | | |
| AF2 (Accessory | 1.0 | 1 | part | 1.0 |
| Assembly) | | | | |

This procedure demonstrates a flexible method for achieving the target 4:1:1 stoichiometric ratio. Instead of encoding the ratio in the DNA (as in Example 9), the ratio is established at the level of delivery by controlling the input copy number of each cassette. This “gene dosage” approach is highly advantageous for optimization, as the delivery ratio (e.g., 2:1:1, 4:1:1, 8:1:1) can be easily tuned without re-cloning the constructs, allowing for rapid empirical determination of the optimal ratio for maximal functional output.

### Example 11: Engineering Stoichiometric Ratios Via Construct Architecture (Polycistronic Vectors)

The purpose of this procedure is to achieve a fixed, non-equal stoichiometric ratio by designing a single construct architecture. This method uses polycistronic linking elements, such as Internal Ribosome Entry Sites (IRES) or 2A peptides, which have known, differential expression-linking properties, to encode a specific ratio from a single promoter.

The objective is to design a single lentiviral vector that, when transcribed as one mRNA, produces gvpA, AF1, and AF2 proteins at a desired non-equal ratio, such as high gvpA and lower AF1/AF2. To achieve this, a polycistronic construct was designed using a combination of linking elements. It is known that an IRES element typically drives expression of a downstream cistron at a much lower level (e.g., 20-50%) than the upstream cistron, while a 2A peptide (like P2A) mediates a nearly 1:1 molar ratio (e.g., 80-99%) via ribosomal skipping. A single vector was engineered with the following architecture: [Promoter]-[gvpA]-[IRES]-[AF1 module]-[P2A]-[AF2 module]. In this configuration, the single promoter (e.g., SFFV) drives high expression of the first cistron, gvpA. The ribosome then encounters the IRES, initiating translation of the AF1 module at a low efficiency (e.g., ˜30\% of gvpA). The ribosome continues, encounters the P2A peptide, and “skips” to translate the AF2 module at a high efficiency relative to AF1 (e.g., ˜ 90\% of AF1).

The corresponding results and Analysis are reported in Table 18

TABLE 18
Engineering DI ratios polynucleotide constructs

| Construct | | Expected Molar Ratio |
|---|---|---|
| Element | Linking Mechanism | (Relative to gvpA = 1.0) |
| gvpA (Cistron 1) | Cap-dependent | 1.0 |
| | translation | |
| AF1 (Cistron 2) | IRES-mediated | ≈0.3 (Significantly |
| | initiation | lower than gvpA) |
| AF2 (Cistron 3) | P2A-mediated skipping | ≈0.27(≈90\% of AF1) |

This example demonstrates how a single construct can be used to encode a fixed, imbalanced stoichiometric ratio (e.t., 1:0.3:0.27). This “construct-encoded” architecture is highly reproducible, as every cell that receives the single vector expresses all components in this fixed ratio. This design permanently achieves a high gvpA:AF ratio, which is functionally similar to the target DI, but sacrifices the flexibility of the multi-vector approaches in Examples 4 and 5.

### Example 12: Quantification of the Transduction-Expression Index (TEI)

The purpose of this procedure is to quantify the Transduction-Expression Index (TEI) of an exemplary multi-construct Gas Vesicle (GV) system. The TEI is a comparative metric that assesses the overall system efficiency, defined as the functional output (e.g., ultrasound signal) normalized by the input delivery (e.g., number of transduced cells). This example compares the optimized 3-vector system (System of Interest) against the 1-vector system (Reference System) to determine the fold-improvement in overall efficiency.

To determine the TEI, two systems were compared in parallel using HEK293T cells. The Reference System (REF) was defined as the 1-vector system, where all 8 gvp genes are on a single large lentiviral construct. The System of Interest (SYS) was defined as the 3-vector system, delivered at the optimized 4:1:1 ratio.

Cells were transduced with each system. For the Reference System, an MOI of 5 was used (which produced the peak signal for that system). For the System of Interest, a total MOI of 10 (at a 4:1:1 ratio) was used.

Two measurements were taken for each sample. First, the “Input” was quantified by measuring the Transduction Efficiency (TrE) via flow cytometry, defined as the percentage of cells positive for the fluorescent reporters linked to the constructs. Second, the “Output” was quantified by harvesting the cells, placing them in a phantom, and measuring the functional ultrasound contrast (BURST Signal-to-Background Ratio, SBR) under Standard Conditions.

The efficiency of each system was calculated by dividing its functional Output (SBR) by its delivery Input (TrE). The TEI was then calculated by dividing the efficiency of the System of Interest by the efficiency of the Reference System. The hypothetical data and calculations are presented in Table 19.

TABLE 19
Efficiency of the system

| | Reference | System of | |
|---|---|---|---|
| | System (1- | Interest (3- | |
| Measurement | vector) | vector) | Notes |
| 1. Functional Output | 3.5 | 118 | The 3-vector system produced |
| (SBR) | | | 33.7-fold more total signal. |
| 2. Delivery Input | 40% | 45% | Delivery efficiency was |
| (TrE) | | | comparable between the two |
| | | | systems. |
| 3. System Efficiency | 3.5/0.40 = | 118/0.45 = | This score represents the SBR |
| (Output/Input) | 8.75 | 262.2 | per percentage point of |
| | | | transduced cells. |
| 4. TEI (SYS Efficiency/ | 262.2/8.75 = | — | The 3-vector system is ≈30- |
| REF Efficiency) | 29.9 | | fold more efficient. |

The System of Interest (3-vector) achieved a Transduction-Expression Index (TEI) of 29.9, demonstrating it is approximately 30-fold more efficient than the 1-vector Reference System. This result highlights that the benefit of the multi-construct system is not simply an improvement in delivery (as the Transduction Efficiency was similar for both systems in this cell type).

Rather, the ˜30-fold gain in TEI indicates that the 3-vector system is vastly more efficient at producing functional, signal-generating GVs after the genes have been delivered. This superior post-transduction efficiency is attributed to the optimized stoichiometric expression achieved by partitioning the genes and controlling their dosage, as defined by the DI ratio.

### Example 13: Optimized Lentiviral Vector Design Enhances GV Gene Cluster Delivery and Expression in Mammalian Cells

To enable their use as acoustic reporters in primary cells, the genes encoding GVs were packaged into lentiviral vectors—the most established delivery vehicle in clinical cell engineering. Lentiviral packaging and transduction efficiency depend heavily on transgene size, with a packaging limit of 8-10 kilobases (kb) and reduced titers with larger payloads in this range [46]. The smallest GV gene cluster expressed in mammalian cells, derived from Anabaena flos-aquae, comprises 8 genes, which take up 4.5 kb without promoters and other genetic elements required for polycistronic expression [47]. To establish a strategy for efficient lentiviral GV expression, compare three vector architectures in which the genes are combined across one, two or three co-transduced viral constructs.

HEK293T cells were transduced with each viral combination at different multiplicities of infection (MOIs) and measured the ultrasound contrast of the cells embedded in hydrogel phantoms (FIG. 7A). two nonlinear ultrasound imaging methods can be used that optimize for GV detection—BURST [44] and xAM [48]. The former makes use of the strong nonlinear scattering produced immediately after collapsing GVs with a high-pressure pulse, while the latter non-destructively captures nonlinear scattering arising from reversible buckling of the GVs with each cycle of the transmitted sound wave.

In the first design, a single lentiviral backbone was used to encode the main structural protein gvpA and all seven GV assembly factor genes (gvpN-1), along with a fluorescent marker (GFP), under a TRE promoter (FIG. 7B). This promoter, used in conjunction with an rtTA transactivator, drives expression in the presence of doxycycline, allowing GV production to be timed with imaging to minimize metabolic burden. GvpA was linked to the assembly factors (AFs) via an internal ribosome entry site (IRES), while the AF genes were themselves polycistronically linked using self-cleaving 2A peptides (iterating through P2A, T2A, E2A, and F2A, to minimize the potential for homology-induced genetic recombination).

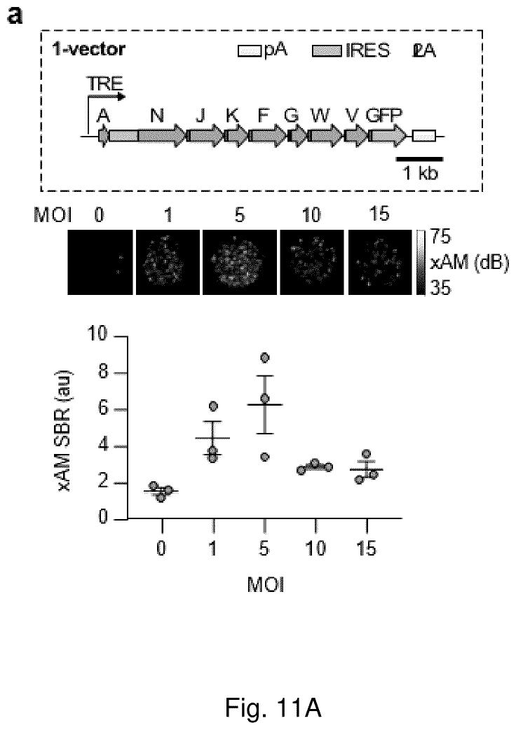

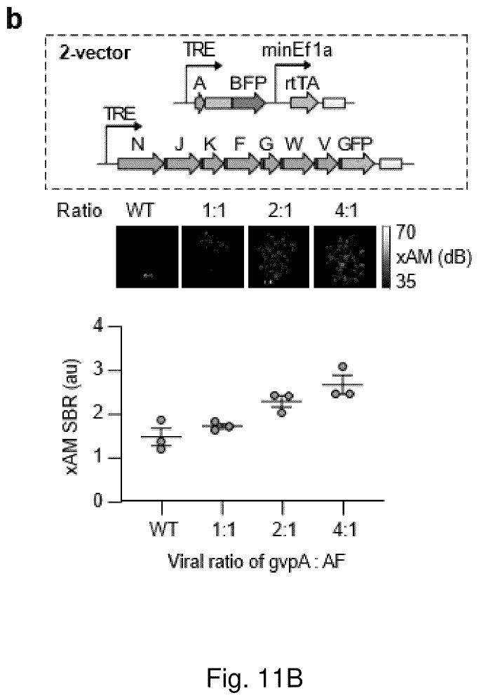

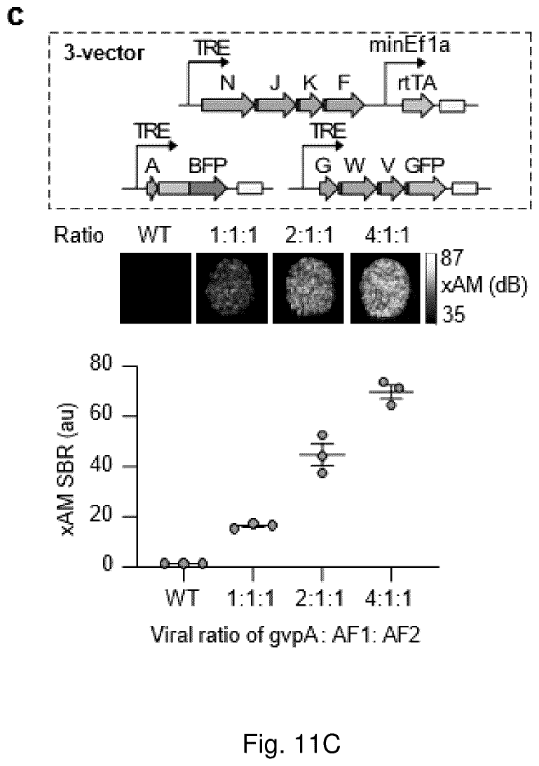

The total packaging size was 8.8 kb. We transduced a HEK-TetON cell line with this virus, while varying the MOI from 1 to 15. We observed BURST ultrasound contrast at all MOIs, with the strongest signal at MOIs of 1, 5, and 10 and decreasing at MOI of 15 (FIG. 7C and FIG. 7D). The xAM contrast was strongest at MOI of 5 and decreased at MOIs of 10 and 15 (FIG. 11A). The highest MOIs also showed reduced infectivity via fluorescent cytometry, suggesting toxicity from high viral burden (FIG. 12A and FIG. 12B). These results show that it is possible to express GVs in mammalian cells using viral vectors, and that the entire GV gene cluster can fit into a single lentiviral construct. While recognizing these important milestones, it is hypothesized that breaking the construct up into two or three smaller inserts would allow more efficient viral packaging and transduction, provide flexibility to control gene stoichiometry, and make room to include the rtTA transactivator to enable expression in wild-type cells.

Thus, the second design comprises two vectors: one encoding the main structural protein gvpA and rtTA, and the second encoding all the AFs, gvpN-I′ (FIG. 7E). These vector inserts had lengths of 5.1 and 8 kb, respectively. This two-vector design enables one to adjust the copy number of gvpA relative to the AFs, a variable shown to impact GV expression in mammalian cells [47]. Transducing a HEK293T cell line, a total MOI of 10 was maintained while varying the MOI ratio between the gypA and the AF vectors. All ratios produced significant ultrasound contrast in BURST (FIG. 7F and FIG. 7G) and xAM (FIG. 11B), peaking at 2:1 and 4:1 ratios, respectively. Transduction was similar across all ratios, remaining above 40%, and the expression of BFP and GFP followed the ratios of the gypA and AF vectors as expected (FIG. 12C and FIG. 12D).

In the third design, split the seven AF genes across two vectors, grouping gypNJKF (AF1) and gvpGWV (AF2), respectively, with packaging sizes of 6.7 and 4.8 kb. GvpA is encoded on its own third virus with an insert length of 4 kb (FIG. 7B). The rtTA was included on the AF1 construct. Using a constant total MOI of 10 while testing three ratios of the gypA vector relative to the two AF vectors, it is observed that a much stronger BURST contrast is produced compared to the one- and two-vector designs (FIG. 7i and FIG. 7J), with the highest signal-to-background ratio (SBR) at the 4:1:1 ratio, measured to be 118±8.4 (mean±s.e.m.). The xAM signal was similarly improved and increased with more gypA (FIG. 11C). Transduction efficiency was comparable to the 2-vector design (FIG. 12E and FIG. 12F). Cell viability was not affected by viral transduction or the induction of GV expression (FIG. 12G). These results demonstrate that a split-vector approach comprising smaller inserts and more ratiometric flexibility provides the best performance for expressing the polycistronic GV complex.

Using the best-performing three-vector transduction system (4:1:1), ultrasound contrast can be quantified as a function of cell concentration after sorting for triple-transduced cells. Significant signal from the cells could be detected at a concentration of 1e6 cells per milliliter with BURST (FIG. 7K) and with xAM (FIG. 7L). This concentration corresponds to approximately 4 cells per image voxel (where each voxel is 0.004 mm³) and a volume fraction of approximately 0.9%. Thus, it is concluded that viral vector delivery enables highly sensitive imaging of GV-expressing cells and adopted the three-vector design for the rest of the experiments

### Example 14: Lentiviral GV Delivery System is Adaptable for Efficient Transduction and Ultrasound Imaging of Immune Cells

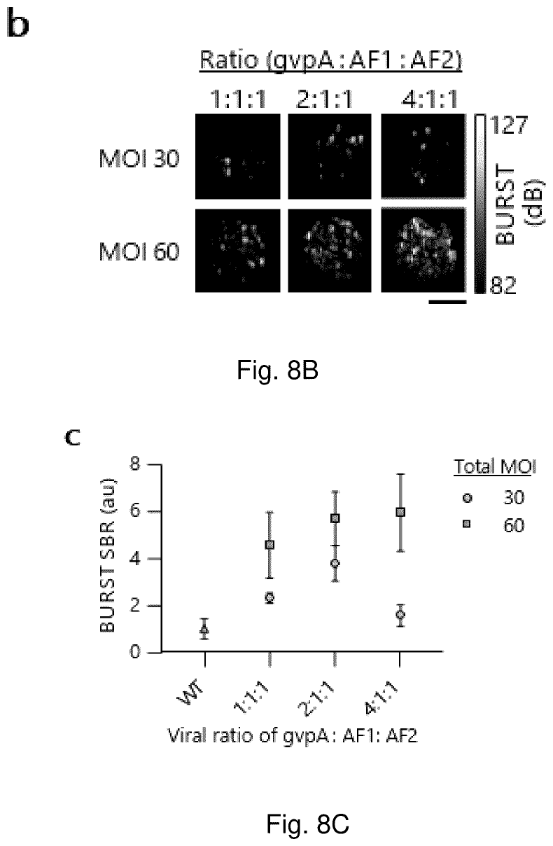

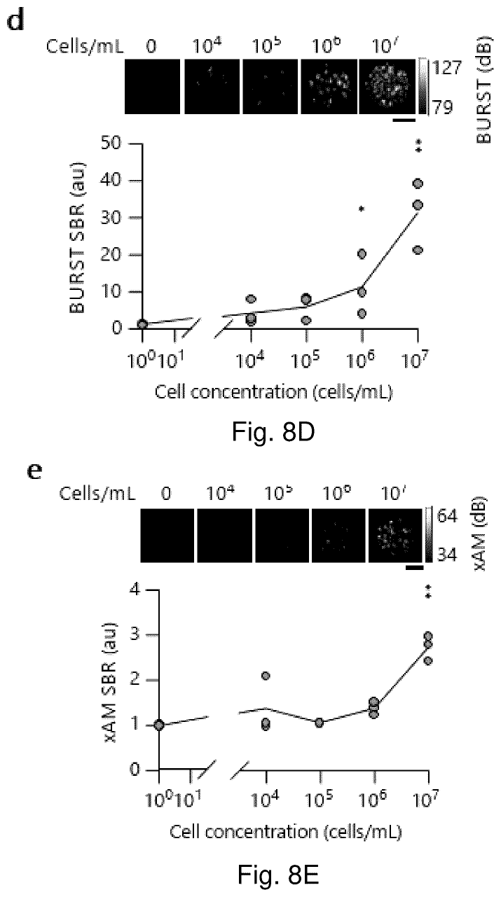

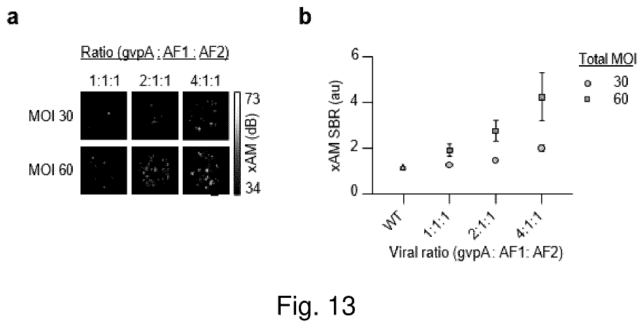

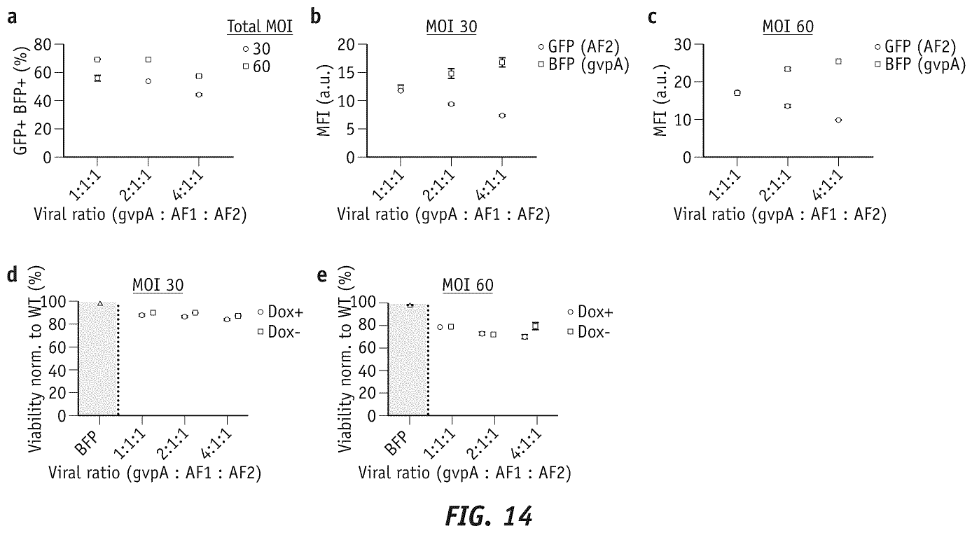

Next the issue was addressed of whether lentiviral GV delivery architecture could be adapted to image immune cells, starting with the Jurkat T cell line, which is commonly used to model lymphocyte-based cell therapies (FIG. 8A). Since Jurkats are more difficult to transduce than HEK239T cells18, it is hypothesized that they would require higher viral doses to achieve similar levels of transduction. The Jurkat cells were incubated with total MOIs of 30 and 60 (as defined based on functional tittering in HEK cells), varying the gvpA-to-AF1 and AF2 ratio within each MOI. While both MOIs resulted in appreciable ultrasound signal at certain ratios, the most consistent BURST contrast was produced at MOI of 60, with the 4:1:1 ratio showing the highest BURST SBR at 9.0±4.5 (FIG. 8 and FIG. 8C FIG. 2b-c), and the strongest signal in xAM (FIG. 13A and FIG. 13B). As expected, cells transduced with the higher MOI had a larger number of triple-transduced cells (FIG. 14A) and higher levels of GFP and BFP expression (FIG. 14C).

Triply transduced and sorted cells in the MOI 60-4:1:1 condition showed significant BURST contrast at concentrations as low as 1e6 cells per milliliter (FIG. 8D). xAM was able to detect as few as 1e7 cells per milliliter (FIG. 8E).

The viability of Jurkat cell lines transduced with the 3-vector system were measured, with and without inducing GV expression. Using 7-AAD (7-aminoactinomycin D) live/dead staining, no significant doxycycline-induced cell death attributed to GV expression was found (FIG. 14D to FIG. 14E). However, there was overall more cell death in cells transduced with the three vectors at MOI 60 compared to MOI 30. This is attributed to the large viral dose, which likely included a large number of empty capsids from packaging larger payloads [3].

These experiments demonstrated the adaptability of our lentiviral architecture for expressing GVs in immune cells, and more generally, in cells considered to be more difficult to transduce.

### Example 15: Acoustic Reporter Genes Enable Monitoring of T Cell Activation

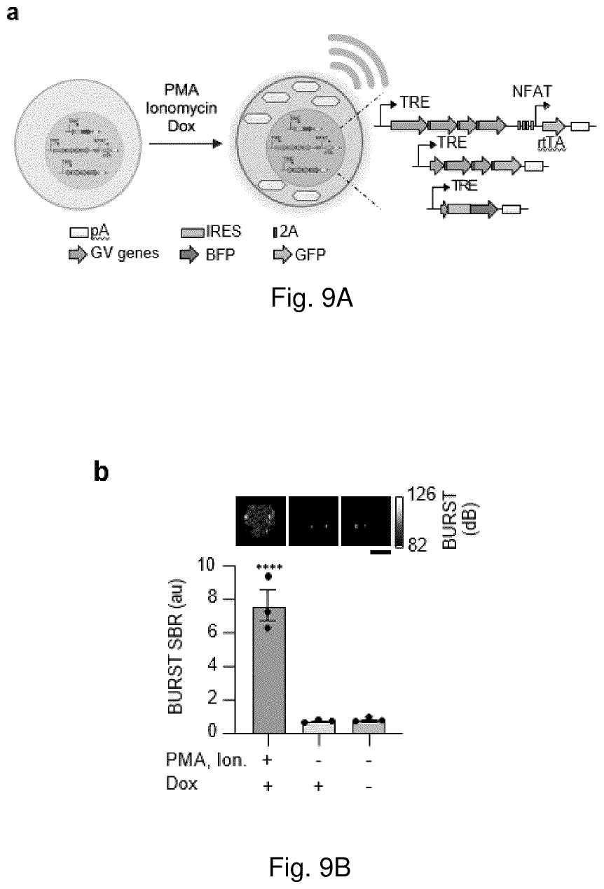

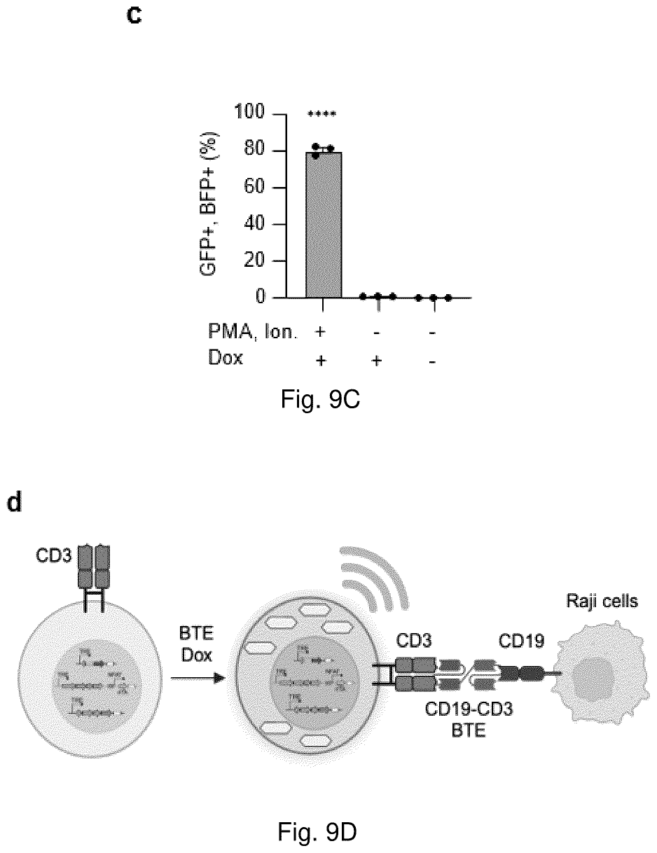

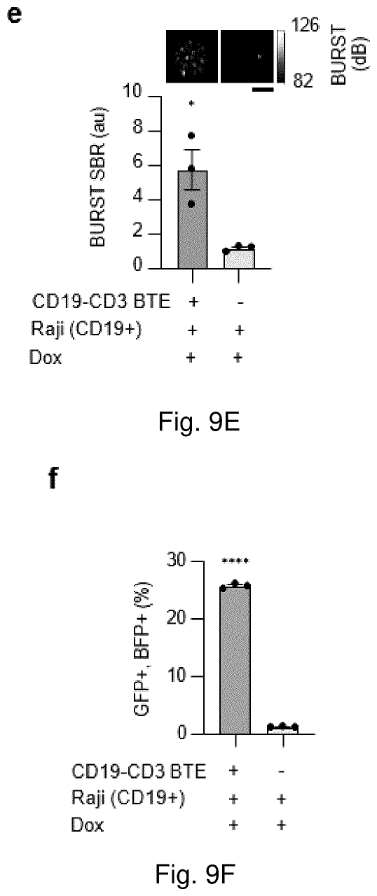

Having engineered immune cells to express GVs, it is hypothesized that this expression could be connected to an activity-dependent promoter as a read-out of cellular state. In particular, seeking to trigger ultrasound contrast in T cells when they become activated by antigen engagement. To achieve this goal, connect GV expression to the NFAT promoter, which activates upon T cell receptor engagement and accompanying calcium influx[3, 49]. A gene circuit was constructed in which the NFAT promoter is placed upstream of the rtTA transactivator, such that NFAT activation initiates transcription of the rtTA, which in the presence of doxycycline drives GV gene transcription (FIG. 9A). This architecture is beneficial because it allows potentially weak NFAT activity to drive a stronger rtTA-TRE transcription complex and temporally confines GV expression to periods when it is needed for imaging, thereby minimizing cellular burden. This architecture is also modular in the sense that only a simple swap of the NFAT promoter is needed to change the conditionality of expression for all 8 GV genes and accompanying fluorophores.

A Jurkat T cell line was created transduced with this engineered three-virus combination and tested whether we can turn on GV expression by chemically inducing the NFAT promoter. When stimulated, these cells with phorbol 12-myristate 13-acetate (PMA) and ionomycin [50] in the presence of doxycycline, they produced strong ultrasound contrast (FIG. 9B). The signal was activity-specific, with a fold-change of 10.9 in BURST SBR when compared with doxycycline alone. Flow cytometry confirmed specific circuit activation, with an activation-dependent fold change of 99.0 in the fraction of cells expressing GFP and BFP (FIG. 9C).

Having confirmed the circuit design, it was tested if receptor-induced activity could be detected with ultrasound when T cells engage with an on-target bait cell. A CD19-CD3 bispecific T-cell engager (BTE) was used, which causes T cell activation when a BTE molecule binds CD3 on the T cell surface and CD19 on the target cell surface at the same time [51]. The NFAT-inducible Jurkat cell line was co-cultured with CD19+ Raji cells at a 1:1 ratio (FIG. 9D). Within 24 hours, in the presence of the BTE, it was observed a large increase of ultrasound contrast (4.8-fold change in BURST SBR) relative to the same co-culture without the molecular engager (FIG. 9E). Flow cytometry corroborated this activation by showing a 22.1-fold change in the GFP and BFP positive population fraction (FIG. 9F). These results highlight the potential of ultrasound to detect cellular activation states by leveraging activity-dependent transcription to drive GV expression.

### Example 16: Ultrasound Monitors In Vivo Homing of GV-Expressing Therapeutic T Cells in Tumors

A major challenge in cell-based therapies is to monitor the trafficking of engineered cells to their intended target tissues after in vivo administration. To address this challenge, use the viral GV expression platform to label a primary T cell therapy and image its homing to tumors following systemic infusion.

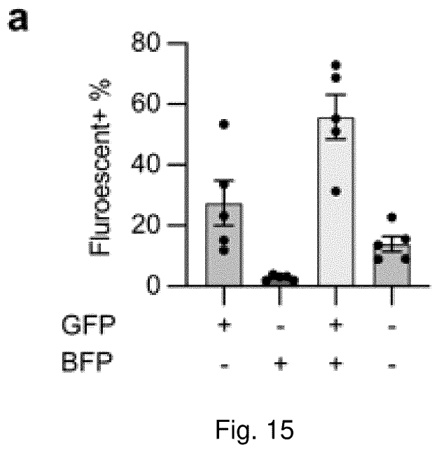

To achieve this goal, isolate primary T cells from human peripheral blood mononuclear cells (PBMCs) and transduce them with the doxycycline-dependent 3-vector system. Since at a total viral titer of MOI 120 (based on functional tittering in HEK cells), less than 20% of these hard-to-transduce cells [52] were triple-positive for all three viruses, using a 1:1:1 ratio to maximize the number of cells with at least one functional unit of each construct (FIG. 10A, top row). After sorting the cells based on GFP expression to ensure that they received all assembly factor genes, and inducing them with doxycycline for 72 hours, a consistent BURST signal was seen in doxycycline-induced T cells of five different human donors that was 3.8±0.2 times (geometric mean of ratios±s.e.m. of log (ratios)) higher than the signal from wild-type non-transduced cells (FIG. 10B), marking the first demonstration of primary mammalian cells expressing acoustic reporter genes. Of the doxycycline-induced sorted cells, 55.8±7.36% (N=5, mean±s.e.m) were both GFP and BFP positive, indicating the presence of both AFs and gvpA in most of this population (FIG. 15).

After demonstrating ultrasound contrast, one may wonder if GV expression affects T cell cytotoxicity, a critical function for administered therapies. To investigate this, the T cells were co-cultured with CD19+ Raji cells at 1:1 and 4:1 effector-to-target (E:T) ratios in the presence of the CD19-CD3 BTE, and measured the percentage of live target cells with 7-AAD staining 24 hours after co-culture. It was found that GV expression (controlled with doxycycline induction) did not affect T cell cytotoxicity at either ratio, and as expected the cells were more effective at killing at the higher E:T ratio and in the presence of the targeting BTE (FIG. 10C). Interestingly, flow cytometry showed increased GFP and BFP expression in co-cultured T cells with BTE engagement, especially at the higher E:T ratio (FIG. 10D). This is because increasing the E:T ratio leads to more efficient target cell lysis, and hence increased T cell activity and protein expression[54] [55-57].

Having demonstrated successful GV expression and ultrasound imaging in vitro, an experiment was designed to show that primary T cells could be imaged in vivo after systemic administration, allowing ultrasound to visualize their trafficking to tumors. The experiment intravenously infused the engineered T cells into immunocompromised mice bearing subcutaneous CD19+ Raji cell flank tumors, followed by intraperitoneal (IP) injections of the CD19-CD3 BTE every 2 days to stimulate activation and local expansion inside the tumor microenvironment[51, 58] . . . . On day 3 after T cell infusion, GV expression was induced with intraperitoneal doxycycline and imaged the tumors with ultrasound (protocol depicted in FIG. 10A, bottom row).

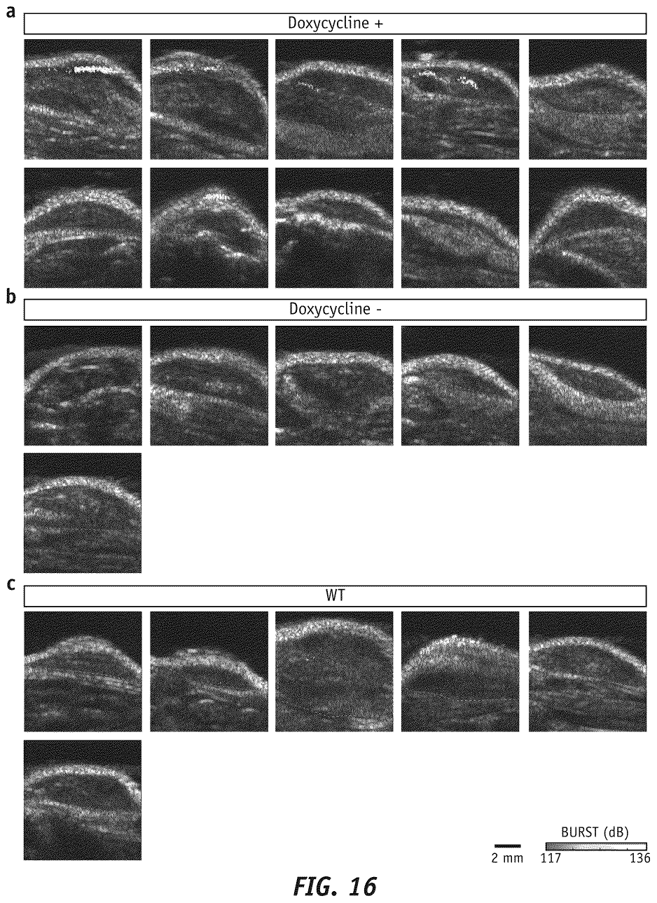

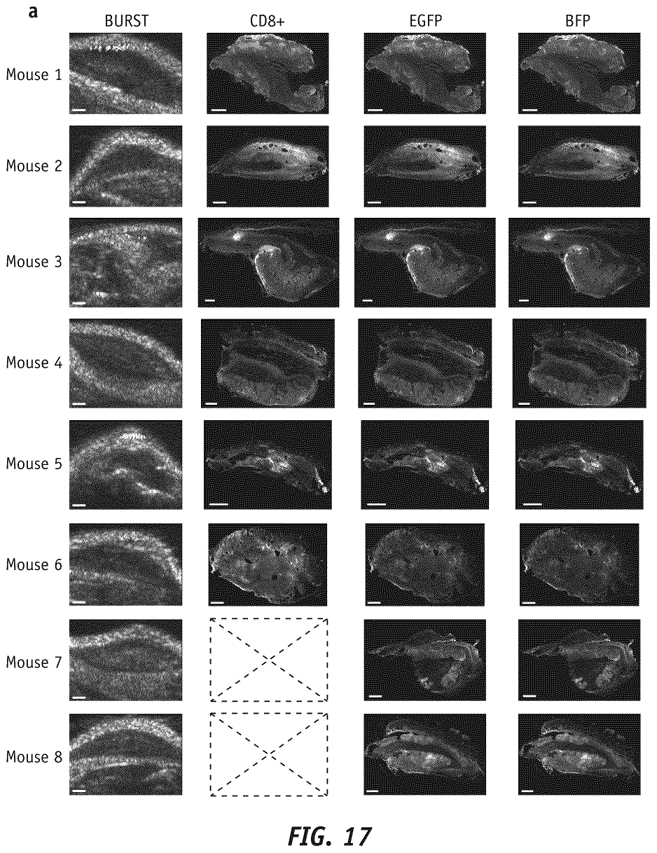

Ultrasound imaging revealed clear BURST ultrasound contrast in the tumors of mice infused with GV-expressing T cells and induced with doxycycline (FIG. 10A to FIG. 10F). This signal was absent in mice that were not doxycycline-induced or received wild-type T cells. This response was consistent across our total cohort of 22 mice, with 4.1 fold-change in BURST SBR for the Dox+ vs. Dox-groups, and 4.3 for the Dox+ vs. WT groups (FIG. 10F and FIG. 16). In a subset of animals, tumors were collected and immunofluorescent imaging was performed to confirm that the localization of ultrasound signal observed in tumors matches the spatial distribution of GV-expressing T cells observed by histology, finding a clear correspondence (FIG. 10G and FIG. 17). For example, in a representative tumor, the core was mostly necrotic with few live cells, while both ultrasound and histology showed cytotoxic T cells expressing the reporter construct concentrated at the viable tumor periphery near the top of the tumor (FIG. 10G to FIG. 10H). The ability of ultrasound to visualize this sub-tumor localization in vivo is in stark contrast to the diffuse signal typically obtained with bioluminescent or nuclear imaging methods.

Notably, tumors with low ultrasound contrast also had few T cells by histology (FIG. 17). Indeed, it was found that there is a strong positive correlation between the absolute number of GFP+ and BFP+ T cells counted in a given histology slice postmortem and the BURST signal measured in the corresponding ultrasound image plane in vivo (r=0.89, p=0.0072, Pearson correlation, FIG. 10I), suggesting that ultrasound can provide a quantitative noninvasive readout of cell therapy homing and/or expansion.

Overall, these experiments demonstrated, for the first time, an ultrasound method to noninvasively visualize the homing of a mammalian cell therapy into its target tissue

### Example 17: Exemplary Applications of the Stoichiometric System

This work introduces a genetic technology platform enabling the use of acoustic reporter genes to image a broader variety of cell types, including primary cells deployed in an in vivo cell therapy context. By devising a set of suitable viral constructs, it is possible to introduce GV genes into transfection-resistant cells such as T cells, with the most effective system comprising a combination of three lentiviral vectors encoding the 8 essential GV genes, along with an rtTA transactivator and fluorescent proteins for correlative histology. In addition, it is shown how this expression system can be easily modified to monitor cellular states using activity-dependent promoters. Furthermore, it was demonstrated that this system enables the noninvasive in vivo monitoring of therapeutic T cell homing and proliferation in tumors with a combination of penetration depth and resolution not available with other modalities.

This acoustic imaging toolkit will significantly advance preclinical studies by allowing the study of the dynamics of cell-based therapies in vivo, such as how adoptively transferred T cells infiltrate solid tumors and where they expand within the tumor microenvironment. Additionally, it could facilitate the monitoring of off-tumor on-target cytotoxicity, a common side effect of CAR T cells by imaging bystander organs. More broadly, this approach opens new possibilities to study the in vivo dynamics of other cell-based therapies, including those based on natural killer (NK) cells, macrophages, stem cells, and any other cells that can be engineered with viruses, and to track cellular states beyond T cell activation, such as during different stages of development. Beyond preclinical research, it is envisioned that the use of something like these acoustic reporter gene constructs in future clinical applications, allowing physicians to check that the therapy they administer reaches the right tissues and performs their intended activities. Such applications would take advantage of the ubiquitous accessibility of ultrasound imaging devices and the emergence of new form factors such as continuously wearable bio-adhesive ultrasound patches31-34.

As expected for the first demonstration of a new imaging approach, this toolkit can benefit from further improvements. The high MOI required for efficient co-transduction of multiple vectors in primary T cells could be burdensome for certain cells and expensive to manufacture at scale. It is anticipated that this limitation will be overcome with future improvements in GV expression efficiency through modified assembly factor stoichiometry 10 or mutagenesis of the GV genes themselves [60]. Further gains could be made by optimizing the expression cassette with improved cell type-specific promoters, targeted genomic integration and addition of elements to reduce epigenetic silencing [61]. Another limitation is that the primary T cells were detectable only with collapse-based BURST imaging, which, while highly sensitive, requires re-expression of GVs for repeated imaging. This is likely because the GVs formed in primary T cells are too small to undergo the reversible mechanical buckling needed for non-destructive xAM imaging38,39. This issue would also be addressed with further gene and protein engineering for more efficient expression. Additionally, using more advanced ultrasound hardware, such as matrix or row-column arrays, could improve the sensitivity and fidelity of xAM40 and BURST imaging by generating a more homogeneous pressure field within the target tissue and minimizing the potential for motion artifacts.

Using the technology developed in this study, ultrasound can now be utilized to image primary mammalian cells, including therapeutic cells, in vivo. With further improvements, getting a quick ultrasound may become as common for checking on a cell therapy as it is for checking on a growing fetus.

Accordingly, provided herein are genetically engineered gas vesicle expression systems (GVES) that are configured to express gas vesicles (GVs) in a mammalian cell, related gas vesicle polynucleotide constructs, gas vesicle reporting genetic circuits, vectors, genetically engineered mammalian cells, non-human mammalian hosts, compositions, methods and systems, which in several embodiments can be used together with contrast-enhanced imaging techniques to detect and report biological events in an imaging target site comprising a mammalian cell and/or organism.

### Example 18: Application of Stoichiometric Gas Vesicle Expression System to Cell Therapy

Cell-based therapies are making breakthrough progress in treating complex diseases such as cancer and autoimmune disease. However, predicting and monitoring their in vivo dynamics, such as tissue infiltration, proliferation and off-target activity, remain challenging and necessitate the development of more advanced in vivo imaging methods. Existing imaging techniques used in research, such as luminescent reporters[62], provide poor spatial resolution due to light scattering, while clinical probes based on positron emission tomography require expensive radioligand synthesis and imaging equipment, and expose patients to radiation[63] [64-66] [67]. Improved imaging techniques are essential for understanding the mechanisms of cell-based therapies, optimizing treatment protocols, and ensuring precise targeting of therapeutic cells to improve their efficacy and reduce off-target effects, ultimately accelerating the development of new cell-based treatments.

Ultrasound presents a promising modality to track cell therapies, as it is affordable, widely available in medical settings, and capable of large-scale scanning of deep organs with spatial resolution on the order of 100 micrometers[68]. To enable in vivo imaging of cell therapies with ultrasound, one would ideally use reporter genes rather than synthetic labels, as this would allow the tracking of proliferating cells without signal dilution and the sensing of specific cellular states via promoter-driven expression.

The stoichiometric GV expression systems here described introduce a method to track cell therapies with ultrasound using acoustic reporter genes based on gas vesicles (GVs). GVs are microbially derived air-filled protein nanostructures that scatter sound waves, enabling their use as reporter genes for ultrasound imaging in mammalian cells[69] [47, 70] and probiotics[11] [47, 71] [72]. However, previous mammalian uses of GVs have been limited to chemical transfections of the GV gene cluster, restricting their use to transfection-compatible cell lines.

in particular, GV gene expression is rather challenging in primary cells due to the difficulty of delivering the 8 or more genes encoding GVs, at appropriate stoichiometry, to such cells using compatible methods such as viral delivery.

The stoichiometric GV expression system herein described address this challenge with optimized and extensively characterized system such as the exemplified lentiviral vector architecture, capable of delivering the complete set of GV-encoding genes into mammalian cells, resulting in robust ultrasound contrast.

The exemplified lentiviral system herein described showed that GV expression can be driven by a chemically inducible promoter or connected to a gene circuit engineered to respond to cell activity, permitting the imaging of receptor-driven immune cell activation. The lentiviral expression system was used to produce GV-expressing primary human T cells and used ultrasound to monitor their accumulation in vivo as an adoptively transferred cell therapy in mouse tumor xenografts, as corroborated by immunohistochemistry. These results position virally-encoded GVs as a prime option for ultrasound imaging of cell-based therapies

In summary, provided herein are stoichiometric Gas Vesicle Expression Systems (GVES), sets of polynucleotide constructs, and related vectors, cells, compositions, and methods configured for robust expression of Gas Vesicle Gene Clusters (GVGCs) in mammalian cells, particularly primary and immune cells. The GVES comprises distinct gene modules for the primary structural protein (gvpA/B) and assembly factors (AF1, AF2), operably configured to achieve a stoichiometric expression ratio, quantified by Dosage Index (DI), wherein the gvpA/B module is expressed at least 2-fold higher than the AF1 and AF2 modules. In preferred embodiments, the modules are provided on separate polynucleotide constructs (e.g., three viral vectors), overcoming packaging limits and enabling efficient co-delivery, characterized by a significantly higher Transduction-Expression Index (TEI) compared to single-construct systems. These systems provide enhanced gas vesicle yield, improved ultrasound contrast (high SBR), and enable non-invasive imaging and tracking of engineered mammalian cells, including therapeutic cells like T cells, in vitro and in vivo.

The examples set forth above are provided to give those of ordinary skill in the art a complete disclosure and description of how to make and use the embodiments of the of the GVES system, polynucleotide constructs for expression of a gas vesicle in mammalian cells, and related GVR genetic circuits, vectors, genetically engineered mammalian cells, compositions, methods and systems of the disclosure, and are not intended to limit the scope of what the inventors regard as their disclosure. Those skilled in the art will recognize how to adapt the features of the exemplified polynucleotide GV constructs, and related genetic circuits, vectors, genetically engineered prokaryotic cells, compositions, methods and systems herein disclosed to additional polynucleotide GV constructs, and related genetic circuits, vectors, genetically engineered mammalian cells, compositions, methods and systems according to various embodiments and scope of the claims.

All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible sub-combinations of the group are intended to be individually included in the disclosure. Every combination of components or materials described or exemplified herein can be used to practice the disclosure, unless otherwise stated. One of ordinary skill in the art will appreciate that methods, system elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the genetic circuits, genetic molecular components, and methods steps set forth in the present description. As will be obvious to one of skill in the art, methods and systems useful for the present methods and systems may include a large number of optional composition and processing elements and steps.

In particular, it will be understood that various modifications may be made without departing from the spirit and scope of the present disclosure. Accordingly, other embodiments are within the scope of the following claims.

### REFERENCES

ADDIN EN.REFLIST 1. Tashiro, Y., et al., Molecular genetic and physical analysis of gas vesicles in buoyant enterobacteria. Environmental microbiology, 2016. 18 (4): p. 1264-1276.
2. Van Keulen, G., et al., Gas vesicles in actinomycetes: old buoys in novel habitats? Trends in microbiology, 2005. 13 (8): p. 350-354.
3. Hogan, P. G., Calcium NFAT transcriptional signalling in T cell activation and T cell exhaustion. Cell calcium, 2017. 63: p. 66-69.
4. Walsby, A. E., Gas vesicles. Microbiol. Rev., 1994. 58 (1): p. 94-144.
5. Walsby, A. E., Gas-vacuolate bacteria (apart from cyanobacteria), in The Prokaryotes. 1981, Springer. p. 441-447.
6. Walsby, A. E., Cyanobacteria: planktonic gas-vacuolate forms. The Prokaryotes, a Handbook on Habitats, Isolation, and Identification of Bacteria, 2013. 1: p. 224-235.
7. Woese, C. R., Bacterial evolution. Microbiological reviews, 1987. 51 (2): p. 221.
8. Walsby, A. E., Gas vesicles. Microbiol Rev, 1994. 58 (1): p. 94-144.
9. Pfeifer, F., Distribution, formation and regulation of gas vesicles. Nat. Rev. Microbiol., 2012. 10 (10): p. 705-15.
10. Yi, G., S.-H. Sze, and M. R. Thon, Identifying clusters of functionally related genes in genomes. Bioinformatics, 2007. 23 (9): p. 1053-1060.
11. Bourdeau, R. W., et al., Acoustic reporter genes for noninvasive imaging of microorganisms in mammalian hosts. Nature, 2018. 553 (7686): p. 86-90.
12. Lakshmanan, A., et al., Preparation of biogenic gas vesicle nanostructures for use as contrast agents for ultrasound and MRI. Nat Protoc, 2017. 12 (10): p. 2050-2080.
13. Hayes, P. and R. Powell, The gypA/C cluster of Anabaena flos-aquae has multiple copies of a gene encoding GvpA. Archives of microbiology, 1995. 164 (1): p. 50-57.
14. Kinsman, R. and P. Hayes, Genes encoding proteins homologous to halobacterial Gvps N, J, K, F & L are located downstream of gvpC in the cyanobacterium Anabaena flos-aquae. DNA Sequence, 1997. 7 (2): p. 97-106.
15. Myers, E. W. and W. Miller, Optimal alignments in linear space. Computer applications in the biosciences: CABIOS, 1988. 4 (1): p. 11-17.
16. Smith, T. F. and M. S. Waterman, Comparison of biosequences. Advances in applied mathematics, 1981. 2 (4): p. 482-489.
17. Needleman, S. B. and C. D. Wunsch, A general method applicable to the search for similarities in the amino acid sequence of two proteins. Journal of molecular biology, 1970. 48 (3): p. 443-453.
18. Pearson, W. R. and D. J. Lipman, Improved tools for biological sequence comparison. Proceedings of the National Academy of Sciences, 1988. 85 (8): p. 2444-2448.
19. Karlin, S. and S. F. Altschul, Methods for assessing the statistical significance of molecular sequence features by using general scoring schemes. Proceedings of the National Academy of Sciences, 1990. 87 (6): p. 2264-2268.
20. Karlin, S. and S. F. Altschul, Applications and statistics for multiple high-scoring segments in molecular sequences. Proceedings of the National Academy of Sciences, 1993. 90 (12): p. 5873-5877.
21. Lu, G. J., et al., Acoustically modulated magnetic resonance imaging of gas-filled protein nanostructures. Nat Mater, 2018. 17 (5): p. 456-463.
22. Pfeifer, F., Distribution, formation and regulation of gas vesicles. Nat Rev Microbiol, 2012. 10 (10): p. 705-15.
23. Li, N. and M. C. Cannon, Gas vesicle genes identified in Bacillus megaterium and functional expression in Escherichia coli. J Bacteriol, 1998. 180 (9): p. 2450-8.
24. Tashiro, Y., et al., Molecular genetic and physical analysis of gas vesicles in buoyant enterobacteria. Environ Microbiol, 2016. 18 (4): p. 1264-76.
25. Ramsay, J. P., et al., A quorum-sensing molecule acts as a morphogen controlling gas vesicle organelle biogenesis and adaptive flotation in an enterobacterium. Proc Natl Acad Sci USA, 2011. 108 (36): p. 14932-7.
26. Schechter, I. and A. Berger, On the size of the active site in proteases. I. Papain. Biochem Biophys Res Commun., 1967. 27 (2): p. 157-162.
27. Schechter, I. and A. Berger, On the active site of proteases. 3. Mapping the active site of papain; specific peptide inhibitors of papain. Biochem Biophys Res Commun., 1968 32 (5): p. 898-902.
28. Calvo, S. E., D. J. Pagliarini, and V. K. Mootha, Upstream open reading frames cause widespread reduction of protein expression and are polymorphic among humans. Proc Natl Acad Sci USA, 2009. 106 (18): p. 7507-12.
29. Rose, A. B., Intron-mediated regulation of gene expression. Curr Top Microbiol Immunol, 2008. 326: p. 277-90.
30. Reddy A. S. N., G. M., Nuclear pre-mRNA Processing in Plants. Current Topics in Microbiology and Immunology. 326: p. 14.
31. Purnick, P. E. and R. Weiss, The second wave of synthetic biology: from modules to systems. Nat Rev Mol Cell Biol, 2009. 10 (6): p. 410-22.
32. Buchler, N. E., U. Gerland, and T. Hwa, On schemes of combinatorial transcription logic. Proceedings of the National Academy of Sciences, 2003. 100 (9): p. 5136-5141.
33. Silva-Rocha, R. and V. de Lorenzo, Mining logic gates in prokaryotic transcriptional regulation networks. FEBS letters, 2008. 582 (8): p. 1237-1244.
34. Terreno, E., et al., Challenges for Molecular Magnetic Resonance Imaging. Chemical Reviews, 2010. 110 (5): p. 3019-3042.
35. Cunningham, C. H., et al., Positive contrast magnetic resonance imaging of cells labeled with magnetic nanoparticles. Magnetic Resonance in Medicine, 2005. 53 (5): p. 999-1005.
36. Foucault, M.-L., et al., In vivo bioluminescence imaging for the study of intestinal colonization by Escherichia coli in mice. Applied and environmental microbiology, 2010. 76 (1): p. 264-274.
37. Daniel, C., et al., Bioluminescence imaging study of spatial and temporal persistence of Lactobacillus plantarum and Lactococcus lactis in living mice. Applied and environmental microbiology, 2013. 79 (4): p. 1086-1094.
38. Chu, J., et al., A bright cyan-excitable orange fluorescent protein facilitates dual-emission microscopy and enhances bioluminescence imaging in vivo. Nat Biotech, 2016. 34 (7): p. 760-767.
39. Smith-Bindman, R., et al., Use of diagnostic imaging studies and associated radiation exposure for patients enrolled in large integrated health care systems, 1996-2010. JAMA, 2012. 307 (22): p. 2400-9.
40. Foster, F. S., et al., Advances in ultrasound biomicroscopy. Ultrasound in medicine & biology, 2000. 26 (1): p. 1-27.
41. Foster, F. S., et al., Principles and applications of ultrasound backscatter microscopy. Ultrasonics, Ferroelectrics and Frequency Control, IEEE Transactions on, 1993. 40 (5): p. 608-617.
42. Errico, C., et al., Ultrafast ultrasound localization microscopy for deep super-resolution vascular imaging. Nature, 2015. 527 (7579): p. 499-502.
43. Chu, J., et al., A bright cyan-excitable orange fluorescent protein facilitates dual-emission microscopy and enhances bioluminescence imaging in vivo. Nature biotechnology, 2016. 34(7): p. 760-767.
44. Sawyer, D. P., et al., Ultrasensitive ultrasound imaging of gene expression with signal unmixing. Nature methods, 2021. 18 (8): p. 945-952.
45. Milone, M. C. and U. O'Doherty, Clinical use of lentiviral vectors. Leukemia, 2018. 32 (7): p. 1529-1541.
46. Yacoub, N. a., et al., Optimized production and concentration of lentiviral vectors containing large inserts. The Journal of Gene Medicine: A cross-disciplinary journal for research on the science of gene transfer and its clinical applications, 2007. 9 (7): p. 579-584 .
47. Hurt, R. C., et al., Genomically mined acoustic reporter genes for real-time in vivo monitoring of tumors and tumor-homing bacteria. Nature Biotechnology, 2023. 41 (7): p. 919-931.
48. Maresca, D., et al., Nonlinear X-wave ultrasound imaging of acoustic biomolecules. Physical Review X, 2018. 8 (4): p. 041002.
49. Jutz, S., et al., Assessment of costimulation and coinhibition in a triple parameter T cell reporter line: Simultaneous measurement of NF-KB, NEAT and AP-1. Journal of immunological methods, 2016. 430: p. 10-20.
50. Chatila, T., et al., Mechanisms of T cell activation by the calcium ionophore ionomycin. The Journal of Immunology, 1989. 143 (4): p. 1283-1289.
51. Goebeler, M.-E. and R. C. Bargou, T cell-engaging therapies—BiTEs and beyond. Nature Reviews Clinical Oncology, 2020. 17 (7): p. 418-434.
52. Cribbs, A. P., et al., Simplified production and concentration of lentiviral vectors to achieve high transduction in primary human T cells. BMC biotechnology, 2013. 13 (1): p. 98.
53. Simon, B., et al., Enhancing lentiviral transduction to generate melanoma-specific human T cells for cancer immunotherapy. Journal of Immunological Methods, 2019. 472: p. 55-64.
54. Wolf, T., et al., Dynamics in protein translation sustaining T cell preparedness. Nature immunology, 2020. 21 (8): p. 927-937.
55. Tan, H., et al., Integrative proteomics and phosphoproteomics profiling reveals dynamic signaling networks and bioenergetics pathways underlying T cell activation. Immunity, 2017. 46 (3): p. 488-503.
56. Ron-Harel, N., et al., Mitochondrial biogenesis and proteome remodeling promote one-carbon metabolism for T cell activation. Cell metabolism, 2016. 24 (1): p. 104-117.
57. Marchingo, J. M. and D. A. Cantrell, Protein synthesis, degradation, and energy metabolism in T cell immunity. Cellular & molecular immunology, 2022. 19 (3): p. 303-315.
58. Nagorsen, D. and P. A. Baeuerle, Immunomodulatory therapy of cancer with T cell-engaging BiTE antibody blinatumomab. Experimental cell research, 2011. 317 (9): p. 1255-1260.
59. Abad, J. L., et al., Single-step, multiple retroviral transduction of human T cells. The Journal of Gene Medicine: A cross-disciplinary journal for research on the science of gene transfer and its clinical applications, 2002. 4 (1): p. 27-37.
60. Hurt, R. C., et al., Directed evolution of acoustic reporter genes using high-throughput acoustic screening. ACS Synthetic Biology, 2024. 13 (7): p. 2215-2226.
61. Cabrera, A., et al., The sound of silence: Transgene silencing in mammalian cell engineering. Cell Systems, 2022. 13 (12): p. 950-973.
62. Santos, E. B., et al., Sensitive in vivo imaging of T cells using a membrane-bound Gaussia princeps luciferase. Nature medicine, 2009. 15 (3): p. 338-344.
63. Tavare, R., et al., Engineered antibody fragments for immuno-PET imaging of endogenous CD8. T cells in vivo. Proceedings of the National Academy of Sciences, 2014. 111 (3): p. 1108-1113.
64. Mall, S., et al., Immuno-PET imaging of engineered human T cells in tumors. Cancer Research, 2016. 76 (14): p. 4113-4123.
65. Wei, W., et al., Noninvasive PET imaging of T cells. Trends in cancer, 2018. 4 (5): p. 359-373.
66. Ashmore-Harris, C., et al., Non-invasive reporter gene imaging of cell therapies, including T cells and stem cells. Molecular Therapy, 2020. 28 (6): p. 1392-1416.
67. Sellmyer, M. A., et al., Imaging CAR T cell trafficking with eDHFR as a PET reporter gene. Molecular Therapy, 2020. 28 (1): p. 42-51.
68. Maresca, D., et al., Biomolecular ultrasound and sonogenetics. Annual review of chemical and biomolecular engineering, 2018. 9 (1): p. 229-252.
69. Farhadi, A., et al., Ultrasound imaging of gene expression in mammalian cells. Science, 2019. 365 (6460): p. 1469-1475.
70. Duan, M., et al., SEMPER: Stoichiometric expression of mRNA polycistrons by eukaryotic ribosomes for compact, ratio-tunable multi-gene expression. Cell Systems, 2024. 15 (7): p. 597-609. e4.
71. Bar-Zion, A., et al., Acoustically triggered mechanotherapy using genetically encoded gas vesicles. Nature Nanotechnology, 2021. 16 (12): p. 1403-1412.
72. Buss, M. T., et al., Probiotic acoustic biosensors for noninvasive imaging of gut inflammation. Nature Communications, 2025. 16 (1): p. 7931.

1. A genetically engineered Gas Vesicle Expression System (GVES) configured for expressing in a mammalian cell a gene cluster of gvp genes (GVGC) encoding GV proteins capable of forming a GV type, the GVES comprising:
a) a gvpA/B differentially expressed gene module comprising a gvpA/B gene under control of a mammalian promoter and additional mammalian regulatory regions configured for expression of a gvpA/B protein in the mammalian cell with a gvpA/B Dosage Index (DI);
b) an AF1 gvp differentially expressed gene module comprising an AF1 set of gvp genes encoding a first set of GV assembly factors AF1, under control of a mammalian promoter and additional regulatory regions configured for expression of the GV assembly factors AF1 in the mammalian cell with an AF1 Dosage Index (DI); and
c) an AF2 gvp differentially expressed gene module comprising an AF2 set of gvp genes encoding a second set of GV assembly factors AF2, under control of a mammalian promoter and additional regulatory regions configured for expression of the GV assembly factors AF2 in the mammalian cell with an AF2 Dosage Index (DI),
wherein the gvpA/B gene module, the AF1 gvp gene module, and the AF2 gvp gene module are operably linked by regulatory sequences allowing co-expression of the GV proteins with a Dosage Index (DI) ratio of gvpA/B-DI:AF1-DI:AF2-DI wherein the gvpA/B-DI is at least 2-fold higher than the AF1-DI and the AF2-DI, thereby enabling formation of the GV type in the mammalian cell.

2. The GVES of claim 1, wherein the DI ratio of gvpA/B-DI:AF1-DI:AF2-DI is in a range from 2:1:1 to 6:1:1.

3.-6. (canceled)

7. The GVES of claim 1, wherein the AF1 set of gvp genes comprises genes encoding GvpN, GvpJ, GvpK, and GvpF.

8. The GVES of claim 1, wherein the AF2 set of gvp genes comprises genes encoding GvpG, GvpW, and GvpV.

9. The GVES of claim 1, wherein the gvp genes are derived from Anabaena flos-aquae.

10. The GVES of claim 1, wherein at least one of the AF1 gvp gene module and the AF2 gvp gene module is comprised within a polycistronic expression cassette, said cassette further comprising a separation element between two or more gvp genes configured to provide separate expression of the corresponding GV proteins.

11. The GVES of claim 10, wherein the separation element is selected from the group consisting of an internal ribosome entry site (IRES) and a 2A self-cleaving peptide sequence.

12.-13. (canceled)

14. The GVES of claim 1, wherein the gvpA/B gene module, the AF1 gvp gene module, and the AF2 gvp gene module are comprised on two or three distinct polynucleotide constructs.

15. The GVES of claim 1, configured for expression in a mammalian cell selected from the group consisting of a primary cell and an immune cell.

16. The GVES of claim 15, wherein the mammalian cell is a T cell.

17. The GVES of claim 1, configured to produce a BURST Signal-to-Background Ratio (SBR) of greater than 75 in vitro under standard assay conditions.

18.-27. (canceled)

28. A genetically engineered mammalian cell comprising the GVES of claim 1 introduced therein.

29. The genetically engineered mammalian cell of claim 28, wherein the cell is a primary cell or an immune cell.

30. The genetically engineered mammalian cell of claim 29, wherein the immune cell is selected from the group consisting of a T lymphocyte, a B lymphocyte, a natural killer (NK) cell, a monocyte, a macrophage, and a dendritic cell.

31. The genetically engineered mammalian cell of claim 29, wherein the cell is a Chimeric Antigen Receptor (CAR) T cell (CAR-T) or a Chimeric Antigen Receptor Phagocyte (CAR-P) cell.

32. The genetically engineered mammalian cell of claim 29, wherein the primary cell is selected from the group consisting of a hepatocyte, a cardiomyocyte, a neuron, an astrocyte, a microglia, a fibroblast, an endothelial cell, a stem cell, and a progenitor cell.

33.-37. (canceled)

38. A method to image a target site comprising a mammalian cell, the method comprising:
a) introducing into the mammalian cell the GVES of claim 1, for a time and under conditions sufficient for formation of the GV type in the mammalian cell; and
b) imaging the target site comprising the mammalian cell by applying ultrasound or a magnetic field to obtain an ultrasound image or an MRI image of the target site.

39. (canceled)

40. The method of claim 38, wherein imaging comprises applying ultrasound, and wherein said applying ultrasound comprises a collapse-based imaging sequence.

41. The method of claim 40, wherein the collapse-based imaging sequence is Burst Ultrasound Reconstruction with Signal Templates (BURST).

42. The method of claim 38, wherein imaging comprises applying ultrasound, and wherein said applying ultrasound comprises a non-linear, non-destructive imaging sequence.

43. The method of claim 42, wherein the non-linear, non-destructive imaging sequence is an Amplitude Modulation Pulse Sequence (xAM).

44.-50. (canceled)

51. The method of claim 38, wherein the GVES or set of polynucleotide constructs is a Gas Vesicle Reporting Molecular Component (GVRMC) comprised within a Gas Vesicle Reporting Genetic Circuit (GVRGC), and wherein formation of the GV type is indicative of a biochemical event sensed by the GVRGC.

52. The method of claim 51, wherein the biochemical event is protease activity, and wherein the GVRMC encodes a GvpC protein comprising a protease cleavage site, such that cleavage of the GvpC by the protease alters the acoustic properties of the GV type, and wherein said imaging detects said alteration in acoustic properties.

53. The method of claim 38, wherein the mammalian cell is a primary cell selected from the group consisting of a hepatocyte, a cardiomyocyte, a neuron, a fibroblast, an endothelial cell, a stem cell, and a progenitor cell.

54. The method of claim 38, wherein the mammalian cell is an immune cell selected from the group consisting of a T lymphocyte, a B lymphocyte, a natural killer (NK) cell, a monocyte, a macrophage, and a dendritic cell.

55.-63. (canceled)

64. The GVES of claim 1, wherein the DI ratio of gvpA/B-DI:AF1-DI:AF2-DI is 5:1:1.


SEQUENCE LISTING (481 sequences)

SEQ ID NO: 1 - protein, 125 aa, synthetic construct
  VARIANT 4: note = X can be any amino acid
  VARIANT 11: note = X can be any amino acid
  VARIANT 14: note = X can be any amino acid
  VARIANT 31: note = X can be any amino acid
  VARIANT 32: note = X can be any amino acid
  VARIANT 34: note = X can be any amino acid
  VARIANT 41: note = X can be any amino acid
  VARIANT 47: note = X can be any amino acid
  VARIANT 59: note = X can be any amino acid
  VARIANT 60: note = X can be any amino acid
  VARIANT 60: note = X can be any amino acid
  VARIANT 89: note = X can be any amino acid
  VARIANT 123: note = X can be any amino acid
RALXYLQAGYXVHXRGPAGTGKTTLAMHLAXXLXRPVMLIXGDDEFXTSDLIGSESGYXXKKVVDNYIHSVVKVEDELRQNWVDNRLTXACREGFTLVYDEFNRSRPEXNNVLLSVLEEKILXLP

SEQ ID NO: 2 - protein, 5 aa, synthetic construct
  REGION 1..5: note = synthetic polypeptide
GSGSG

SEQ ID NO: 3 - protein, 48 aa, synthetic construct
  REGION 1..48: note = synthetic polypeptide
  VARIANT 16: note = X can be any amino acid
SSSLAEVLDRILDKGXVIDAWARVSLVGIEILTIEARVVIASVDTYLR

SEQ ID NO: 4 - protein, 6 aa, synthetic construct
  REGION 1..6: note = synthetic polypeptide
LDRILD

SEQ ID NO: 5 - protein, 16 aa, synthetic construct
  REGION 1..16: note = synthetic polypeptide
  VARIANT 7: note = X can be any amino acid
RILDKGXVIDAWARVS

SEQ ID NO: 6 - protein, 5 aa, synthetic construct
  REGION 1..5: note = synthetic polypeptide
DTYLR

SEQ ID NO: 7 - protein, 6 aa, synthetic construct
  REGION 1..6: note = synthetic polypeptide
VVNMSV

SEQ ID NO: 8 - protein, 19 aa, synthetic construct
  REGION 1..19: note = synthetic polypeptide
KSPDSSSLAEVLDRILDKG

SEQ ID NO: 9 - protein, 36 aa, synthetic construct
  REGION 1..36: note = synthetic polypeptide
VIDAWARVSLVGIEILTIEARVVIASVDTYLRYAEA

SEQ ID NO: 10 - protein, 6 aa, synthetic construct
  REGION 1..6: note = synthetic polypeptide
NRLDLE

SEQ ID NO: 11 - protein, 308 aa, synthetic construct
  REGION 1..308: note = synthetic polypeptide
MTVLTDKRKKGSGAFIQDDETKEVLSRALSYLKSGYSIHFTGPAGGGKTSLARALAKKRKRPVMLMHGNHELNNKDLIGDFTGYTSKKVIDQYVRSVYKKDEQVSENWQDGRLLEAVKNGYTLIYDEFTRSKPATNNIFLSILEEGVLPLYGVKMTDPFVRVHPDFRVIFTSNPAEYAGVYDTQDALLDRLITMFIDYKDIDRETAILTEKTDVEEDEARTIVTLVANVRNRSGDENSSGLSLRASLMIATLATQQDIPIDGSDEDFQTLCIDILHHPLTKCLDEENAKSKAEKIILEECKNIDTEEK

SEQ ID NO: 12 - protein, 255 aa, synthetic construct
  REGION 1..255: note = synthetic polypeptide
MSETNETGIYIFSAIQTDKDEEFGAVEVEGTKAETFLIRYKDAAMVAAEVPMKIYHPNRQNLLMHQNAVAAIMDKNDTVIPISFGNVFKSKEDVKVLLENLYPQFEKLFPAIKGKIEVGLKVIGKKEWLEKKVNENPELEKVSASVKGKSEAAGYYERIQLGGMAQKMFTSLQKEVKTDVFSPLEEAAEAAKANEPTGETMLLNASFLINREDEAKFDEKVNEAHENWKDKADFHYSGPWPAYNFVNIRLKVEEK

SEQ ID NO: 13-14 - skipped (2 entries)

SEQ ID NO: 15 - protein, 19 aa, synthetic construct
  REGION 1..19: note = synthetic polypeptide
ATNFSLLKQAGDVEENPGP

SEQ ID NO: 16 - protein, 18 aa, synthetic construct
  REGION 1..18: note = synthetic polypeptide
EGRGSLLTCGDVEENPGP

SEQ ID NO: 17 - protein, 20 aa, synthetic construct
  REGION 1..20: note = synthetic polypeptide
QCTNYALLKLAGDVESNPGP

SEQ ID NO: 18 - protein, 22 aa, synthetic construct
  REGION 1..22: note = synthetic polypeptide
VKQTLNFDLLKLAGDVESNPGP

SEQ ID NO: 19 - protein, 22 aa, synthetic construct
  REGION 1..22: note = synthetic polypeptide
DVFRSNYDLLKLCGDIESNPGP

SEQ ID NO: 20 - protein, 22 aa, synthetic construct
  REGION 1..22: note = synthetic polypeptide
TLTRAKIEDELIRAGIESNPGP

SEQ ID NO: 21 - protein, 6 aa, synthetic construct
  REGION 1..6: note = synthetic polypeptide
GAPGSG

SEQ ID NO: 22 - protein, 8 aa, synthetic construct
  REGION 1..8: note = synthetic polypeptide
LEVLFQGP

SEQ ID NO: 23 - protein, 5 aa, synthetic construct
  REGION 1..5: note = synthetic polypeptide
DDDDK

SEQ ID NO: 24 - protein, 4 aa, synthetic construct
  REGION 1..4: note = synthetic polypeptide
IEGR

SEQ ID NO: 25 - protein, 7 aa, synthetic construct
  REGION 1..7: note = synthetic polypeptide
ENLYFQG

SEQ ID NO: 26 - protein, 6 aa, synthetic construct
  REGION 1..6: note = synthetic polypeptide
LVPRGS

SEQ ID NO: 27 - protein, 10 aa, synthetic construct
  REGION 1..10: note = synthetic polypeptide
DLEVVTSTWV

SEQ ID NO: 28 - protein, 10 aa, synthetic construct
  REGION 1..10: note = synthetic polypeptide
DEMEECASHL

SEQ ID NO: 29 - protein, 10 aa, synthetic construct
  REGION 1..10: note = synthetic polypeptide
DCSTPCSGSW

SEQ ID NO: 30 - protein, 10 aa, synthetic construct
  REGION 1..10: note = synthetic polypeptide
EDVVCCSMSY

SEQ ID NO: 31 - protein, 9 aa, synthetic construct
  REGION 1..9: note = synthetic polypeptide
DEMEECSQH

SEQ ID NO: 32 - DNA, 72 bp, synthetic construct
  misc_feature 1..72: note = synthetic polynucleotide
  source 1..72: mol_type = other DNA
ggagcgccaggttccggggctactaacttcagcctccttaaacaggccggcgacgtggaagagaatcctggc

SEQ ID NO: 33 - protein, 72 aa, synthetic construct
  REGION 1..72: note = synthetic polypeptide
  VARIANT 46: note = X can be any naturally occurring amino acid
  VARIANT 65: note = X can be any naturally occuring amino acid
  VARIANT 72: note = X can be any naturally occurring amino acid
MAVEKTNSSSSLAEVIDRILDKGIVIDAWVRVSLVGIELLAIEARXVIASVETYLKYAEAVGLTXSAAVPAX

SEQ ID NO: 34 - protein, 71 aa, Aphanizomenon flos-aquae
MAVEKTNSSSSLAEVIDRILDKGIVIDAWVRVSLVGIELLAIEARIVIASVETYLKYAEAVGLTQSAAVPA

SEQ ID NO: 35 - protein, 72 aa, Aphanothece halophytica
MAVEKTNSSSSLGEVVDRILDKGVVVDLWVRVSLVGIELLAVEARVVVASVETYLKYAEAVGLTSSAAVPAE

SEQ ID NO: 36 - protein, 71 aa, Anabaena flos-aquae; identical to SEQ ID NO: 34

SEQ ID NO: 37 - protein, 70 aa, Ancylobacter aquaticus
MAVEKINASSSLAEVVDRILDKGVVVDAWVRVSLVGIELLAVEARVVVAGVDTYLKYAEAVGLTASAQAA

SEQ ID NO: 38 - protein, 70 aa, Aquabacter spiritensis
MAVEKINASSSLAEVVDRILDKGVVVDAWVRVSLVGIELLAVEARVVVAGVDTYLKYAEAVGLTAGAQAA

SEQ ID NO: 39 - protein, 72 aa, Arthrospira sp.
  source 1..72: note = PCC-8005
MAVEKVNSSSSLAEVIDRILDKGIVIDAWVRVSLVGIELLSVEARVVIASVETYLKYAEAVGLTAQAAVPSV

SEQ ID NO: 40 - protein, 71 aa, Calothrix sp.
  source 1..71: note = Strain PCC-7601
MAVEKTNSSSSLAEVIDRILDKGIVVDAWVRVSLVGIELLAIEARIVIASVETYLKYAEAVGLTQSAAVPA

SEQ ID NO: 41 - protein, 72 aa, Dactylococcopsis salina
  source 1..72: note = PCC-8305
MAVEKTNSSSSLGEVVDRILDKGVVVDLWVRVSLVGIELLAVEARVVIASVETYLKYAEAVGLTSSAAVPAE

SEQ ID NO: 42 - protein, 71 aa, Dolichospermum circinale; identical to SEQ ID NO: 34
  source 1..71: note = AWQC131C

SEQ ID NO: 43 - protein, 71 aa, Dolichospermum lemmermannii; identical to SEQ ID NO: 34

SEQ ID NO: 44 - protein, 70 aa, Enhydrobacter aerosaccus
MAVEKMNASSSLAEVVDRILDKGIVIDAWVRVSLVGIELLAVEARVVVAGVDTYLKYAEAVGLTAGAEAA

SEQ ID NO: 45 - protein, 72 aa, Lyngbya confervoides
  source 1..72: note = BDU141951
MAVEKVNSSSSLAEVVDRILDKGIVVDAWVRVSLVGIELLAIEARVVIASVETYLKYAEAVGLTAQAAVPAS

SEQ ID NO: 46 - protein, 73 aa, Nostoc punctiforme
  source 1..73: note = PCC-73102
MAVEKVNSSSSLAEVIDRILDKGIVIDAWVRVSLVGIELLSIEARIVIASVETYLRYAEAVGLTSQAAVPSAA

SEQ ID NO: 47 - protein, 71 aa, Nostoc sp.
  source 1..71: note = PCC-7120
MAVEKTNSSSSLAEVIDRILDKGIVVDAWVRVSLVGIELLAIEARIVIASVETYLKYAEAVGLTQSAAMPA

SEQ ID NO: 48 - protein, 71 aa, Microchaete diplosiphon; identical to SEQ ID NO: 40

SEQ ID NO: 49 - protein, 71 aa, Microcystis aeruginosa
  source 1..71: note = NIES-843
MAVEKTNSSSSLAEVIDRILDKGIVIDAWARVSLVGIELLAIEARVVIASVETYLKYAEAVGLTQSAAVPA

SEQ ID NO: 50 - protein, 71 aa, Microcystis aeruginosa; identical to SEQ ID NO: 49
  source 1..71: note = NIES-843

SEQ ID NO: 51 - protein, 71 aa, Microcystis aeruginosa; identical to SEQ ID NO: 49
  source 1..71: note = NIES-843

SEQ ID NO: 52 - protein, 71 aa, Microcystis flos-aquae; identical to SEQ ID NO: 49
  source 1..71: note = TF09

SEQ ID NO: 53 - protein, 72 aa, Phormidium tenue
  source 1..72: note = NIES-30
MAVEKVNSSSSLAEVVDRILDKGIVIDAWVRVSLVGIELLAIEARVVIASVDTYLKYAEAVGLTAQAAVPAA

SEQ ID NO: 54 - protein, 72 aa, Planktothrix agardhii
MAVEKVNSSSSLAEVIDRILDKGIVIDAWVRVSLVGIELLSIEARIVIASVETYLKYAEAVGLTAQAAVPSV

SEQ ID NO: 55 - protein, 72 aa, Planktothrix rubescens; identical to SEQ ID NO: 54

SEQ ID NO: 56 - protein, 72 aa, Pseudanabaena galeata
  source 1..72: note = PCC-6901
MAVEKVNSSSSLAEVIDRILDKGIVIDAWVRVSLVGIELLSIEARVVIASVETYLKYAEAVGLTASAAVPAA

SEQ ID NO: 57 - protein, 70 aa, Stella vacuolata
MAVEKINASSSLAEVVDRILDKGVVVDAWVRVSLVGIELLAVEARVVVAGVDTYLKYAEAVGLTAGAQTA

SEQ ID NO: 58 - protein, 75 aa, Trichodesmium erythraeum
  source 1..75: note = IMS101
MAVEKVNSSSSLAEVIDRILDKGVVVDAWIRLSLVGIELLTIEARIVVASVETYLKYAEAVGLTTLAAAPGEAAA

SEQ ID NO: 59 - protein, 72 aa, Trichodesmium erythraeum
  source 1..72: note = IMS101
MAVEKVNSSSSLAEVIDRILDKGVVVDAWVRLSLVGIELLTIEARIVIASVETYLKYAEAVGLTTLAAEPAA

SEQ ID NO: 60 - protein, 71 aa, Tolypothrix sp.; identical to SEQ ID NO: 40
  source 1..71: note = PCC-7601

SEQ ID NO: 61 - protein, 71 aa, Tolypothrix sp.; identical to SEQ ID NO: 40
  source 1..71: note = PCC-7601

SEQ ID NO: 62 - protein, 78 aa, synthetic construct
  REGION 1..78: note = synthetic polypeptide
  VARIANT 29: note = X can be any naturally occurring amino acid
  VARIANT 73: note = X can be any naturally occurring amino acid
MAQPDSSSLAEVLDRVLDKGVVVDVWARXSLVGIEILTVEARVVAASVDTFLHYAEEIAKIEQAELTAGAEAXPAPEA

SEQ ID NO: 63 - protein, 76 aa, Halobacterium salinarum
MAQPDSSGLAEVLDRVLDKGVVVDVWARVSLVGIEILTVEARVVAASVDTFLHYAEEIAKIEQAELTAGAEAAPEA

SEQ ID NO: 64 - protein, 79 aa, Halobacterium salinarum
MAQPDSSSLAEVLDRVLDKGVVVDVWARISLVGIEILTVEARVVAASVDTFLHYAEEIAKIEQAELTAGAEAPEPAPEA

SEQ ID NO: 65 - protein, 76 aa, Halobacterium salinarum; identical to SEQ ID NO: 63
  source 1..76: note = NRC-1

SEQ ID NO: 66 - protein, 79 aa, Halobacterium salinarum; identical to SEQ ID NO: 64
  source 1..79: note = NRC-1

SEQ ID NO: 67 - protein, 78 aa, Haloferax mediterranei
  source 1..78: note = ATCC-33500
MVQPDSSSLAEVLDRVLDKGVVVDVWARISLVGIEILTVEARVVAASVDTFLHYAEEIAKIEQAELTAGAEAAPTPEA

SEQ ID NO: 68 - protein, 78 aa, Halogeometricum borinquense
  source 1..78: note = DSM-11551
MAQPDSSSLAEVLDRVLDKGVVVDVWARVSLVGIEILTVEARVVAASVDTFLHYAEEIAKIEQAELTATAEAAPTPEA

SEQ ID NO: 69 - protein, 76 aa, Halopenitus persicus; identical to SEQ ID NO: 63
  source 1..76: note = strain DC30

SEQ ID NO: 70 - protein, 72 aa, Haloquadratum walsbyi
  source 1..72: note = C23
MAQPDSSSLAEVLDRVLDKGIVVDTFARISLVGIEILTVEARVVVASVDTFLHYAEEIAKIEQAELTAGAEA

SEQ ID NO: 71 - protein, 78 aa, Halorubrum vacuolatum
  source 1..78: note = strain DSM-8800
MAQPDSSSLAEVLDRVLDKGVVVDVYARLSLVGIEILTVEARVVAASVDTFLHYAEEIAKIEQAELTAGAEAAPTPEA

SEQ ID NO: 72 - protein, 95 aa, Halopiger xanaduensis
MAQPQRRPDSSSLAEVLDRILDKGVVIDVWARISVVGIELLTIEARVVVASVDTFLHYAEEIAKIEQATAEGDLEELEELEVEPRPESSPQSAAE

SEQ ID NO: 73 - protein, 95 aa, Natrialba magadii
  source 1..95: note = ATCC-43099
MAQPQRRPDSSSLAEVLDRVLDKGVVIDIWARVSVVGIELLTVEARVVVASVDTFLHYAEEIAKIEQATAEGDLEDLEELEVEPRPESSPKSATE

SEQ ID NO: 74 - protein, 94 aa, Natrinema pellirubrum
  source 1..94: note = DSM-15624
MAQPQRRPDSSSLAEVLDRVLDKGVVIDVWARISVVGIELLTIEARVVVASVDTFLHYAEEIAKIEQATAEGDLDELEELEVEPRPESSPKSAE

SEQ ID NO: 75 - protein, 95 aa, Natronobacterium gregoryi
  source 1..95: note = SP2
MAQPQRRPDSSSLAEVLDRILDKGVVIDVWARVSVVGIELLTIEARVVVASVDTFLHYAEEIAKIEQATAEGDLEDLEELEVEPRPESSPQSATE

SEQ ID NO: 76 - protein, 74 aa, Methanosaeta thermophila
MVTSTPDSSSLAEVLDRILDKGIVVDVWARVSLVGIEILTVEARVVVASVDTFLHYSEEMAKIEQAAIAAAPSA

SEQ ID NO: 77 - protein, 76 aa, Methanosaeta thermophila
MVTSTPDSSSLAEVLDRILDKGIVVDVWARVSLVGIEILTVEARVVVASVDTFLHYSEEMAKIEQAAIAAAPGVPA

SEQ ID NO: 78 - protein, 76 aa, Methanosarcina barkeri-3
MVSQSPDSSSLAEVLDRILDKGIVVDVWARVSLVGIEILAIEARVVVASVDTFLHYAEEITKIEIAAKEEKPAIAA

SEQ ID NO: 79 - protein, 76 aa, Methanosarcina vacuolata
MVSQSPDSSSLAEVLDRILDKGIVVDTWARVSLVGIEILAIEARVVVASVDTFLHYAEEITKIEIAAREEKPVIAA

SEQ ID NO: 80 - protein, 76 aa, Methanosarcina vacuolata
MVSQSPDCSSLAEVLDRILDKGIVVDTWARVSLVGIEILAIEARVVVASVDTFLHYAEEITKIEIAAREEKPVIAA

SEQ ID NO: 81 - protein, 87 aa, Haladaptatus paucihalophilus
  source 1..87: note = DX253
MVQAEPNSSSLADVLDRILDKGVVIDVWARISVVGIEVLTVEARVVVASVDTFLHYAKEMAKLERASSEDEIDFEQVEVASPEASTS

SEQ ID NO: 82 - protein, 73 aa, synthetic construct
  REGION 1..73: note = synthetic polypeptide
  VARIANT 8: note = X can be any naturally occurring amino acid
  VARIANT 34: note = X can be any naturally occurring amino acid
  VARIANT 73: note = X can be any naturally occurring amino acid
MSIQKSTXSSSLAEVIDRILDKGIVIDAFARVSXVGIEILTIEARVVIASVDTWLRYAEAVGLLDVEEGLPRX

SEQ ID NO: 83 - protein, 86 aa, Bacillus megaterium
MSIQKSTDSSSLAEVIDRILDKGIVIDAFARVSLVGIEILTIEARVVIASVDTWLRYAEAVGLLTDKVEEEGLPGRTEERGAGLSF

SEQ ID NO: 84 - protein, 88 aa, Bacillus megaterium
MSIQKSTNSSSLAEVIDRILDKGIVIDAFARVSVVGIEILTIEARVVIASVDTWLRYAEAVGLLRDDVEENGLPERSNSSEGQPRFSI

SEQ ID NO: 85 - protein, 72 aa, synthetic construct
  REGION 1..72: note = synthetic polypeptide
  VARIANT 31: note = X can be any naturally occurring amino acid
  VARIANT 67: note = X can be any naturally occurring amino acid
MAKVQKSTDSSSLAEVVDRILDKGIVIDAWXKVSLVGIELLSIEARVVIASVETYLKYAEAIGLTAXAAAPA

SEQ ID NO: 86 - protein, 73 aa, Burkholderia sp.
  source 1..73: note = Bp5365
MAKVQKSTDSSSLAEVVDRILDKGIVIDVWAKVSLVGIELLSIEARVVIASVETYLKYAEAIGLTATAAAPTA

SEQ ID NO: 87 - protein, 72 aa, Desulfobacterium vacuolatum
  source 1..72: note = DSM-3385
MAKVQKTTDSSSLAEVVDRILDKGIVVDAWAKISLVGIELISIEARVVIASVETYLKYAEAIGLTAAAAAPA

SEQ ID NO: 88 - protein, 72 aa, Desulfomonile tiedjei
  source 1..72: note = DSM-6799
MAKIAKSTDSSSLAEVVDRILDKGIVIDAWAKVSLVGIELLSVEARVVIASVETYLKYAEAIGLTASAAAPA

SEQ ID NO: 89 - protein, 72 aa, Isosphaera pallida
  source 1..72: note = ATCC-43644
MAKVTKSTDSSSLAEVVDRILDKGIVIDAFAKVSLVGIELLSVEARVVIASVETYLKYAEAIGLTASAATPA

SEQ ID NO: 90 - protein, 72 aa, Lamprocystis purpurea
  source 1..72: note = DSM-4197
MAKVANSTDSSSLAEVVDRILDKGIVIDAWIKVSLVGIELLAIEARIVIASVETYLKYAEAIGLTAPAAAPA

SEQ ID NO: 91 - protein, 72 aa, Lamprocystis purpurea
  source 1..72: note = DSM-4197
MAKVANSTDSSSLAEVVDRILDKGIVIDAWLKVSLVGIELLAVEARVVIASVETYLKYAEAIGLTAPAAAPA

SEQ ID NO: 92 - protein, 72 aa, Legionella drancourtii
  source 1..72: note = LLAP12
MAKVQKSTDSSSLAEVIDRILDKGIVIDVWAKVSLVGIELLSIEARVVIASVETYLKYAEAIGLTATASHPA

SEQ ID NO: 93 - protein, 72 aa, Psychromonas Ingrahamii
MANVQKTTDSSGLAEVIDRILDKGIVIDAFVKVSLVGIELLSIEARVVIASVETYLKYAEAIGLTASAATPA

SEQ ID NO: 94 - protein, 72 aa, Psychromonas Ingrahamii
MANVQKSTDSSGLAEVVDRILEKGIVIDAFVKVSLVGIELLSIEARVVIASVETYLKYAEAIGLTASAATPA

SEQ ID NO: 95 - protein, 72 aa, Serratia
  source 1..72: note = 39006
MAKVQKSTDSSSLAEVVDRILDKGIVIDAWVKVSLVGIELLSIEARVVIASVETYLKYAEAIGLTASAATPA

SEQ ID NO: 96 - protein, 72 aa, Thiocapsa rosea
  source 1..72: note = strain DSM-235-Ga0242571-11
MAKVANSTDSSSLAEVVDRILDKGIVIDAWVKVSLVGIELLAIEARVVIASVETYLKYAEAIGLTAPAAAPA

SEQ ID NO: 97 - protein, 70 aa, Bradyrhizobium oligotrophicum
  source 1..70: note = S58
MAIEKATASSSLAEVIDRILDKGVVIDAFVRVSLVGIELLSIELRAVVASVETWLKYAEAIGLVAQPMPA

SEQ ID NO: 98 - protein, 71 aa, Desulfotomaculum acetoxidans
  source 1..71: note = DSM-771
MAVKHSVASSSLVEVIDRILEKGIVIDAWARVSLVGIELLAIEARVVVASVDTFLKYAEAIGLTKFAAVPA

SEQ ID NO: 99 - protein, 67 aa, Octadecabacter antarcticus
  source 1..67: note = 307
MAVNKMNSSSSLAEVVDRILDKGVVIDAWVRVSLVGIELIAVEARVVIAGVDTYLKYAEAVGLTAEA

SEQ ID NO: 100 - protein, 67 aa, Octadecabacter arcticus
  source 1..67: note = 238
MAVSKMNSSSSLAEVVDRILDKGVVIDAWVRVSLVGIELIAVEARVVIAGVDTYLKYAEAVGLTAEA

SEQ ID NO: 101 - protein, 68 aa, Pelodictyon luteolum
  source 1..68: note = DSM-273
MAVEKTIGSSSLVEVIDRILDKGVVVDAWVRMSLVGIELLAIEARVVVASVETYLKYAEAIGLTAKAA

SEQ ID NO: 102 - protein, 68 aa, Pelodictyon luteolum
  source 1..68: note = DSM-273
MAVEKTIGSSSLVEVIDRILDKGVVVDAWVRVSLVGIELLAIEARVVVASVETYLKYAEAIGLTAKAA

SEQ ID NO: 103 - protein, 68 aa, Pelodictyon phaeoclathratiforme
MSVEKTIGSSSLVEVIDRILDKGVVVDAWVRVSLVGIELLAIEARVVVASVETYLKYAEAIGLTAKAA

SEQ ID NO: 104 - protein, 70 aa, Rhodobacter capsulatus
  source 1..70: note = SB-1003
MAIEKSLASASIAEVIDRVLDKGIVVDAFVRISLVGIELLAIELRAVVASVETWLKYAEAIGLTVDPQTP

SEQ ID NO: 105 - protein, 70 aa, Rhodobacter sphaeroides
MAIEKSVASASIAEVIDRILDKGVVIDAFVRVSLVGIELIAIEVRAVVASIETWLKYAEAVGLTVDPATT

SEQ ID NO: 106 - protein, 245 aa, Anabaena flos-aquae
MSIPLYLYGIFPNTIPETLELEGLDKQPVHSQVVDEFCFLYSEARQEKYLASRRNLLTHEKVLEQTMHAGFRVLLPLRFGLVVKDWETIMSQLINPHKDQLNQLFQKLAGKREVSIKIFWDAKAELQTMMESHQDLKQQRDNMEGKKLSMEEVIQIGQLIEINLLARKQAVIEVFSQELNPFAQEIVVSDPMTEEMIYNAAFLIPWESESEFSERVEVIDQKFGDRLRIRYNNFTAPYTFAQLDS

SEQ ID NO: 107 - protein, 268 aa, Ancylobacter aquaticus
  source 1..268: note = strain UV5
MSATLSAPGTANVAVEATAAADGKYLYGIIEAPAPATFDVPAIGGRGDVVHTIALGRLAAVVSNSPRIDYDNSRRNMLAHTKVLEAVMARHTLLPVCFGTVGSDAEVIIEKILRERRDELAGLLGQMHGRMELGLKASWREEIIFEEVLAENPAIRKLRDALVGRSPDQSHYERIQLGERIGQALQRKRQDDEERILERVRPFVHKTRLNKLIGDRMVINAAFLVDAAVESRLDASIRAMDEEWGGRLAFKYVGPVPPYNFVTITIHW

SEQ ID NO: 108 - protein, 245 aa, Aphanizomenon flos-aquae
  source 1..245: note = NIES-81
MNTGLYLYGIFPDPIPETVDLQGLDKQSVHSQVVDGFSFLYSDACQEKYLASRRNLLTHEKVLEQAMHEGFHVLLPLRFGLVVKDWETIQKQLIEPYKEQLNELFQKLAGQREVSIKILWDSKSELQAMMESNQDLKQQRDNMEGKKLKMEEIIQIGQLIESNLAARKQTVIQEFFNNLHPLAKEIIESEPMTEEMIYNAAFLIPWETESVFSERVEAIDRKFGDRLRIRYNNFTAPYTFAQLAS

SEQ ID NO: 109 - protein, 245 aa, Aphanothece halophytica
  source 1..245: note = strain PCC 7418
MAEGFYLYGIFPPPGPQTIAVQGLDKQPIFSHTVEGFTFLYSEAQQSRYLASRRNLITHTKVLEEAMEQGFRTLLPLQFGLVVPDWESVSQDLLQHQSETLQLLFQRLEGKREVSLKIYWETDAELNALLEENPDLKARRDNLEGKNLSMDEVIQIGQALEQAMERRKQEVITRFEDALIPFAVETQENDVLTETMIYNTAFLIPWESEPEFGEAVETVDAEFAPRLKIRYNNFTPPYNFVELRE

SEQ ID NO: 110 - protein, 261 aa, Aquabacter spiritensis
  source 1..261: note = strain DSM 9035
MMQTDTLAPAETVAEGKYLYCLIDAPAPDTFASPGIGGRGDVVHTITVGRLAAVVSDSPRIEYENSRRNMMAHTKVLEEVMARHTMLPVCFGTVATGPDPISGKILEGRRDELVGLLEQMRGRLELGLKATWREDVIFAEILQENPAIAKLRDSLVGRSPEKSHFERIRLGEMIGQAMERKRRDDEERILERVRPFVHKTKLNKPIGDRMILNAAVLVEAAREAGLDQAVRQMDAEWGARLSFKYVGPVPPYNFVTITIHW

SEQ ID NO: 111 - protein, 255 aa, Bacillus-megaterium; identical to SEQ ID NO: 12

SEQ ID NO: 112 - protein, 258 aa, Bradyrhizobium oligotrophicum
  source 1..258: note = S58
MSNQPIYVYGLIRAEDHQPLAVRAVGDSEQPVNIIGSGNVAALVSTIDLPEIMPTRRHMLAHTKVLEAAMANGPVLPMRFGIIVPNPATLLRVIGFRHQELRARLDEIDGRIEVALKASWDEQFMWRQLASEHPDLAVSGRTMMGRGEQQSYYDRIELGRAIGAALEERRTAARLQLLQTVTPFAVQVKELTPVDDAMFAHLALLVEKGAEPSLYQTVEALERSNDSGLKFRYVAPIPPYNFVAVTLDWEQHEQAPRR

SEQ ID NO: 113 - protein, 249 aa, Burkholderia thailandensis
  source 1..249: note = sp. Bp5365 strain MSMB43
MNSRNGARYLYAVQHARDVPASLPAGIGGAAVRALTDGDVAAIVSDTGLAKVRPERRHLLAHHTVIQSLAAAGTVLPVAFGTIATSEVALRRMLRKHRNALAGELARLVDHVEMSVRLNWDVTDLFRHLIDVRPDLKAARDAMLALGSAVTRDDKIELGSRFERVLNEERARHAALVDEALDACCKEIRRDPPRHETEILHLTCLVRHAELGRFESGVAAASRELDDSLVLKYSGPCPPHHFVNLNMSL

SEQ ID NO: 114 - protein, 260 aa, Chlorobium luteolum
  source 1..260: note = DSM 273
MERDGKYIYCIIGADCECDFGPIGIGGRGDLVSTIGFEGISMVVSDHPLNRFVVDPDGILAHQRVIEAVMKEHESVIPVRFGTVAATPDEIRNLLDRRYGELSELLLRLRNKVEFNVTGRWHDMAAIYKEVERTHPEIKEQRARIESMRDGDGEALKQSLILDTGHQIEAALEVMKEEKFDAVASLFRKTAMASKMNRTTSPDMFMNAAFLIDRGREVEFDGIMEILGQKDADRCDYRYSGPLAIFNFVDLRILPEKWEL

SEQ ID NO: 115 - protein, 259 aa, Chlorobium luteolum
  source 1..259: note = DSM 273
MAHEAAEQDGLYIYGIINNSGELDFGPIGIGGREERVYAVIHNDIAAVVSRTVVKEFEPRRANMIAHQKVLEAVMVSHAVLPVRFSTVSPGHDDMKVEKILEEDYLRLKKLLVKMEGKKEMGLKVMANEEKVYESIITGYDNIRYLRDKLINLPPEKTHYQRVKIGELVAAALEKEVGTYKDAVLDALSPIAEEVKVNDSYGSMMVLNAAFLIRTAREEEFDRAVNALDDRYHDMMTFKYVGTLPPYNFVNISINIKGR

SEQ ID NO: 116 - protein, 343 aa, Chlorobium luteolum
  source 1..343: note = DSM 273
MNQSIYIYGIVNEPALAASFVETDPDIYAVASMGCSAIVENRPAIDLGELDRESLARMLLQHQQTLERLMESGMQLIPLKLGTFVSSAADAACIIEDGYNLIERIFRETEDAHELEVVVKWSSFADLLQEVVSEGDVQELKREVEARQSSSTEDAIAVGRLIKEKIDRRNAALSASVLRQLGERASQSKRHETMDDEMVLNAAFLVNRGDVDAFVATVEALDSQYLNALHFRIVGPLPCYSFYTLEVTALFEEFIAEKRAVLGLDARSCEADVKKAYHAKAKVAHPDVHVPAGANNGADFTVLNEAYMTLHDYYSALRNSASSRHGHEGQDSSSVVFSVKILN

SEQ ID NO: 117 - protein, 245 aa, Dactylococcopsis salina
  source 1..245: note = PCC 8305
MTEGFYLYGIFPPPGPKTIETQGLDKQPIFSHTVEGFTFLYSEAQQSRYLASRRNLITHTKVLEEAMENGSRTLLPLQFGLIVPDWETVVQDLLQHQAESLHFFLEKLEGKREVSLKIYWETNAELNALLEENPALKARRDNLEGKQLSMDEVIQIGQALEQEMEGRKQDIISRFEEVLIPFAFEIKENDVLTETMIYNTAFLINWDAESDFGEQLEAIDAEFSPRLKIRYNNFTPPYNFVELRE

SEQ ID NO: 118 - protein, 252 aa, Desulfobacterium vacuolatum
  source 1..252: note = DSM 3385
MSKKNLKRNGRYLYAIIEASEEKTFGSIGMDGSDVYLIVEDKTAAVVSDVPNKKIRPQRKNIAAHHAVLNKIMEEITPLPMAFGIIADGEQAIRKILADNRDVFREQFATVSGKVEMGMRISYDVPNIFEYFISTDSEIRAARDQYFGGNREPSQEAKLELGRMFNRQLNANREEYTNQVIEILDDYCDDIKENKCRNEQEVTSLACLINRSDQKRFEEGVFESARHFDNNFSFEYNGPWSPHNFVNILIEL

SEQ ID NO: 119 - protein, 255 aa, Desulfomonile tiedjei
  source 1..255: note = DSM 6799
MEKATIKTTGSNGRYLYAVVPGSQERVYGCLGINGGNVYTIAAKDVAAVVSDVPHQKIRPERRHFAAHQAVLKRVMLDGDLLPMSFGIISQGPKAVRAILSRNNKSVQQQLKRISGKAEMGIKVTWDVPNIFEYFIDVNRELREARNKLVQPNYLPTQQEKIEIGRMFEEILNLERERHTKQVERVMSKRCSEIKRSKCRTEIEVMNLSCLVDRTLLSDFEAGVLEAASHFDDSFAFDFNGPWAPHNFVDLEIDV

SEQ ID NO: 120 - protein, 250 aa, Desulfotomaculum acetoxidans
  source 1..250: note = DSM 771
MSTGRYVYCVINSIEPLTFMSGPVGNEPEGVFTVHYKELAAVVSQSSEEKYNVCRENTIAHQKVLEEVLVSHPLLPVRFGTVAQNEEIVKKFLLQERYAELRSMLHNVTGKVQMGLKVLWTDMKTVYQEIVEENPQIKNLKKKLESKPAETIHYEMIDLGQMVNQALLRKKEKQKEMVLKPLQKIALETKESFLYGDQMFVNADFLISRSSLDDFNAKVNELGEFFNEQALFKYIGPLPPYNFVTLYVNF

SEQ ID NO: 121 - protein, 357 aa, Desulfotomaculum acetoxidans
  source 1..357: note = DSM 771
MVKNHNTDHLKELYIYGLIGGTPFKDELEKISVIQENTPIYGVWHKNIGFAVSAAPDYPLKDLSKESIIQLFVDHQQVLECLRQKFSLIPVKLGTVLESVTEAAAVLANNEEKFNDLLNYLKDKVELNLSVSWNDLNEVVAKIGEEDEVKKLKQSLLAQEQVSQEDLIKIGKIISFQMQQKKQAAREYIISELRNLWEDYFINEVVDENSILNLTLLAITGKVDDVNKKIEYLNQIYRDSLDFSLTKSLLPQGFSTVSIKKITMDQLLLAKDILKLPDTASLQDINAARRALLHCYHPDKNDHAAVNKVQEINAAYKLLEEYCQENSSDFNVDLITDYYIMKVIKADKSNVNSMNME

SEQ ID NO: 122 - protein, 252 aa, Dolichospermum circinale
MNTDLAHKNFGLYLYGIFPDTIPETLEIKGLDGKSVHSQVVDGFTFLYSQACQEKYLASRRNLLAHERVLEQTMHEGFHVLLPLRFGLVVKDWETIMSQLINPHKEQLHKLFEKLAGQREVSIKILWDAKAELQAMMESNHDLRQQRDNMEGKKLSMEEVIQIGQLIESNLQARKQAVIEVFTRELNPLAQEIVVSEPMTEEMIYNAAFLIPWDSEPLFSERVESIDQKFGNRLRIRYNNFTAPYTFALLDS

SEQ ID NO: 123 - protein, 277 aa, Enhydrobacter aerosaccus
  source 1..277: note = strain ATCC 27094
MNPPEAYIAGRTAAKSVEDRKARPQDLAEGKYVYAIIACDEPREFKNRGIGERGDKVHTINHRQMAAVVSDSPTIDYERSRRNMMAHTVVLEEVMKEFDLLPLRFGTVASSAESVERQLLVPRYGELSAMLEKMRGRSEFGLKAFWHEGVAFGEIVRENARVRKLRDALQGRSLEESYYQRIQLGEEVEKALTAIRARDEELILSRLRPFMRDIRTNKIISDRMVLNAAFLVERGDVPALDEAIRQLDQEFSERLMFKYVGPVPPYNFVNIAINWER

SEQ ID NO: 124 - protein, 279 aa, Isosphaera pallida
  source 1..279: note = ATCC-43644
MRNAPPTRPGSVTPASPGKPVIDGPARYLYAFTHDLPEGPLADLEGLPGARVVVVADGRVAAVVSPCPLGKVRPERQRVAGHHHVLKHLQDTLGKAILPASFGMVADSEEDLRALLRHHSAAIAEGLVRVQGKVEMTVKLRWAPDNVAQAVLGRDPELRQLRDQLYSNGQTPTRDQSLDLGRRFHHALERQRDHYAAYLRAALSPLLSELVEEDLRDERDLVHWACLIENQRRAGFEAALDRLAEELEDDLVLELTGPWPPHHFVDLDLDDDHDDDEEE

SEQ ID NO: 125 - protein, 258 aa, Legionella drancourtii
  source 1..258: note = LLAP12
MDSTSKKPAASNLYLYAIASVNENQEPISFHGIEEQPIDLVPYKDIMLVVSNLSKKKVRPERKNVAVHHAVLNHLMKHNTSMLPIRFGMIADNRKEVQRLLTINYDMLHTKLKMMAGRVEMGVSLSWDVPNIFEYLLNRHSQLRETRDKLLANPAHEPSRDEKIEIGALFSQILDEEREVYTDTILSLLSPVCCDVVKSTYRNDTEIMNIFCLISAARRDEFEEKIIEASTILDDNFVIKYTGPWPPHNFSKLNLSLE

SEQ ID NO: 126 - protein, 247 aa, Lyngbya confervoides
  source 1..247: note = BDU141951
MPQLLYLYGIFPAPGPQDLEVQGLDQQPIHTHIIDEFVFLYSVAQQERYLASRKNLLGHERVLEAAMKVGYRTLLPLQFGLIIETWDRVIKELITPRGDALKRLFAKLEGRREVSVKLLWGPDAELNQLMEEDAGLRAERDRLEGQQLSMDQIVDIGQAIETAMTERKDDVINAFRQRLNALAIEVLENDPLTDAMIYNTAYLIPWEDEVKFSQAIEELDEQFEDRLRIRYNNFTAPYNFAQLDQLS

SEQ ID NO: 127 - protein, 244 aa, Microcystis aeruginosa
  source 1..244: note = NIES-843
MTVGLYLYGIFPEPVPDGLVLQGIDNEPVHSEMIEGFSFLYSAAHKEKYLASRRYLICHEKVLETVMEAGFTTLLPLRFGLVIKTWESVTEQLISPYKTQLKELFAKLSGQREVSIKIFWDNQWELQAALESNPKLKQERDAMMGKNLNMEEIIHIGQLIEATVLQRKQDIIQVFRDQLNHRAQEVIESDPMTDDMIYNAAYLIPWEQEPEFSQNVEAIDQQFGDRLRIRYNNLTAPYTFAQLV

SEQ ID NO: 128 - protein, 247 aa, Nostoc punctiforme
  source 1..247: note = ATCC 29133
MSFYIYGILTLPAPQNLNLEGLDRQPVQIKILDDFAVIYSEAQQERYLASRRNLLSHEKVLEEIMQAGDRYLLPVQFGLLVSSWETVSQQLIRPHQEELTQLLAKLSGCREVSVKVFWDTEAEIQGLLAEHPNLKTERDKLVGQPLSMERVIQIGQVIEQGMSDRKQGIIDVFKGTLNSIAIEVVENTPQVDTMIYNSAYLIPWEAESQFSEHVESLDRQFENRLRIRYNNFTAPYNFARLRLTTSN

SEQ ID NO: 129 - protein, 244 aa, Nostoc sp.
  source 1..244: note = PCC 7120
MSSGLYLYGIFPDPIPETVTLQGLDSQLVYSQIIDGFTFLYSEAKQEKYLASRRNLISHEKVLEQAMHAGFRTLLPLRFGLVVKNWETVVTQLLQPYKAQLRELFQKLAGRREVSVKIFWDSKAELQAMMDSHQDLKQKRDQMEGKALSMEEVIHIGQLIESNLLSRKESIIQVFFDELKPLADEVIESDPMTEDMIYNAAFLIPWENESIFSQQVESIDHKFDERLRIRYNNFTAPYTFAQIS

SEQ ID NO: 130 - protein, 264 aa, Octadecabacter antarcticus
  source 1..264: note = 307
MKREVVRMTDENTINSKYLYAIIKCREQREFIARGIGERGDAVHTIAYKGLAAVVSDSPVMEYDQSRRNMMAHTAVLEELMEEFTLLPVRFNTVAPEAGAIEERLLVPRHEEFTQLLGQIDKRVELGIKAFWHDGMIFEEVLRENDSIRKMRDALEGKSVDGSYYERIQLGEKIEQAMIKKRVEDEEIILSRIRQHVHKSRSNKTIGDRMVLNGAFLVDANKESDFDKAVQLLDQDLGNRLMFKYVGPVPPYNFVNIVVNWGVV

SEQ ID NO: 131 - protein, 356 aa, Octadecabacter antarcticus
  source 1..356: note = 307
MTVVAEENMTGSVGLYVCAIVAEWESNSALIKCANEAQGEIQLIGQGGITAVVMVPPEDQPVSRDRQELVRQLLVHQQLVERFTEIAPVLPVKFGTLAPDRESVELGLERGREKFFTAFGGLSGKTQFEITVTWDVADVFAKIAKLPAVVKLKVDLVATSESDRPINLDRVGRLVKETLDHQRAQTGKVLLDALLPLGVDSIVNPILNDSIVLNLALLVDTDQADALDRCLDELDSTFHGALSFRCVGPMPPHSFATVEINYIEPTQVSHACCVLELDAAHNFEEIRSAYHRLARQTQQDIAPDVVVDNKSSSVGIAVLNDAYKTLLSFVDAGGPVVVSVQRQEDAYATDIPSSGG

SEQ ID NO: 132 - protein, 257 aa, Octadecabacter arcticus
  source 1..257: note = 238
MTDEKKVNSKYLYAIIQCREPRELKARGIGERGDVVHTVVHKGLAAVVSDSPVMEYDQSRRNMMAHTAVLEELMEEFTLLPVRFNTVAPEAVAIEERLLVPRHDEFTQLLGQIDKRVELGLKAFWHDGMIFGEVLRENDSIRKMRDSLKGQSVDGSYYERIQLGEKIEKALTEKRLEDEEMILSRIRPHVHKSRSNKTIGDRMVLNGAFLVDAEKESKFDEAVQSLDQDLSDRLMFKYVGPVPPYNFVNIVVNWGES

SEQ ID NO: 133 - protein, 360 aa, Octadecabacter arcticus
  source 1..360: note = 238
MRAQKVIPAAEENISGNVGLYVCAIVAERVSCSALIQCANDAPGEIQLIGHGDFTAVVMVPEKDQLVSPDRKELMQQLLVHQQLIEKFMEIAPVLPVKFATLAPNRESVELGLEVGSEKFSAAFNSLSGKVQFEVIVTWDVAEVFAEIAKEPAVAKLKVDLAAMPESYGSVSLEQLGKLVKETLELRRAETGKVLLDALVQVGVDNVVNSILDDSIILNLALLVEAKRADAFDRCLDELDSTYHGALTFRCVGPLPPHSFATVEITYLEPAKVTEACDILELDVARSTEEVRSAYHRLARKSHPDIVPDVAVGETASVSMAVLTDAYKTLLSFVGAGGSVVVSVQRQEASYAADIISSAG

SEQ ID NO: 134 - protein, 258 aa, Pelodictyon phaeoclathratiforme
MDIETTKEGRYIYGIIRNSEFIDFGQIGIGKRNDRVYGVIYKDICAVVSSTPIIQYEARRANMIAHQKVLEEVMKRFNVLPVRFSTISPHDNDDAIIKILITDYSRFDELLIKMKGKKELGLKVMADETRIYENIIQKYDNIRSLRDKLLNQPADKIHYQRVKIGEMVADALKKEIESYKQQILDILSPIAEDIKITDNYGNLMILNAAFLIKEVKESEFDDSVNKLDEKYGNIMTFKYVGTLPPYNFVNLSINTKGV

SEQ ID NO: 135 - protein, 257 aa, Pelodictyon phaeoclathratiforme
MEKDGKYVYCIIASTYECNFGAIGIGGRGDLVNTIGFQGLSMVVSDHPLNHFVLNPDNILAHQRVIEVVMSQFNSVIPVRFGTVAATPDEIRNLLDRRYGELSELLERFENKVEYNLKASWRCMIDIYKEIDKEHVELKQLRREIEGLKDEEKRKLLIVEAGHIIENELQKKKEVEAYEIVTYLRKTVVAHKHNKTTGEAMFMNTAFLLNKGREVEFDNIMNDLGEQYKDRSDYYYTGPLPIFNFIDLRILPEKWEL

SEQ ID NO: 136 - protein, 339 aa, Pelodictyon phaeoclathratiforme
MDRQGIYIYGFIPNHYLTDIKTILIESGIYSIEYGSIAALVSDTMVDDIEYLNREDLAYLLVDHQKKIELIMSTGCSTIIPMQLGTIVNSGNDVIKIVKNGLRIINKTFDDIADIQEFDLVVMWNNFPDLIKKISDTPQIRIMKEEIANKGSYDQADSINIGKIIKKKIDEKNSKVNLDIMNSLSSLCICVKKHESMNDEMPLNSAFLIKKDKENSFIEMVNQLDIKYENLLRYKIVGPLPCYSFYTLESKLLNKKEIEKAEKILGIDAYKSESDIKKAYRAKAAHAHPDKNNTISAIDNDDFIEINKAYQILLEYSSVFKDSPDHKPDEPFYLVKIKK

SEQ ID NO: 137 - protein, 244 aa, Phormidium tenue
  source 1..244: note = NIES-30
MADRYYLYGIFPAPGPAELPLMGLDEQVVQAQQLGDFTFLYSLACQKRYLSSRKNLLGHEKVLEAAMEQGHRTLLPLQFGLIVESWNQVQEDLVTPYAEDLTQLFGRLNGCREVSIKVQWEPSTELEMMMAENADLRAQRDQLEGTQLGMEQVIFIGQQIESALEERKQGIVDQFRQALSPLAKDVLENAPQTDVMIYNAAFLIPWESEAEFSQAVDAIDSTFGDRLRIRYNNFTAPYNFAQLN

SEQ ID NO: 138 - protein, 245 aa, Planktothrix agardhii
  source 1..245: note = str. 7805
MGNGLYLYGILPTNRVRPLALHGLDKQPIQTHPVDEFSFLYSETQQERYLASRRNLLGHEDVLEKVMQHGYRSVLPLQFGLIVKDWDHVKAQLIIPYQDRLKELFHKLEGKREVGVKIFWEETEELDLLMTENQELREKRDSLEGKRLSMDEIIGIGQEIERAMQDRQQGIIDKFQQILNPLAQEIVENDNLTSAMIYNAAYLIPWDIEPQFGDKIEELDHHFNNRLRIRYNNFTAPFNFAQLNP

SEQ ID NO: 139 - protein, 268 aa, Psychromonas ingrahamii
  source 1..268: note = 37
MAENKKKVRKSSSKVIAKPKVIYAITAGGLQDLGNLVGINKSDIYTIEKESISFVVSDLSPSSPRPRPDRRNIMAHNEILKQLMSKTSVLPVRFGTVATGERAVNRFCSQYNAQLLEQLDRVQDRVEMGIKVTWNVPNIYDYFVDNHSELREERDRVYDGNKNPRRDDRINLGHMYDALVTEARLSHQTDLEEIILPGCDEIHSIPPKDEKVVVNLACLVQRADLEVFEERVVEAGKTLDNTYDIELNGPWAPHNFVELDLKTMTGRR

SEQ ID NO: 140 - protein, 273 aa, Serratia sp.
  source 1..273: note = ATCC 39006
MMSIDKSRNHRAKVLYALCVSDDSTPNYKIRGLEAAPVYSIDQDGLRAVVSDTLSTRLRPERRNITAHQAVLHKLTEEGTVLPMRFGVIARNAEAVKNLLVANQDTIREHFERLDGCVEMGLRVSWDVTNIYEYFVATYPVLSETRDEIWNGNSNANNHREEKIRLGNLYESLRSGDRKESTEKVKEVLLDYCEEIIENPVKKEKDVMNLACLVARERMDEFAKGVFEASKLFDNVYLFDYTGPWAPHNFVTLDLHAPTAKKKTLTRAGTLSD

SEQ ID NO: 141 - protein, 260 aa, Stella vacuolate
  source 1..260: note = ATCC-43931
MQTEALAPAAVAAEGKYLYCIIDAPAPATFASPGIGGRGDVVHTLAVGRLAAVVSDTPRIEYENSRRNMMAHTKVLEEVMAHHTLLPVCFGTVGSGDDVIAEKILEGRREELSRLLEEMRGRVELGLKATWREEVIFAEVLDEDPAVRKLRDSLVGRSPEKSHFERIRLGELIGQALLRKRRDEEERILDRVRPFVRKTKLNKPIGDRMILNAAFLVETAREAALDQSVREMDADWGARLSFKYVGPVPPYNFVTITIHW

SEQ ID NO: 142 - protein, 254 aa, Thiocapsa rosea
  source 1..254: note = strain DSM 235
MQQAKRQDVAAGRYIYAIIPDRGDHSLGRIGLDESEVYTIGDGRVAAVVSDLSGGRIRPQRRNMAAHQEVLKQVLREVSPLPAAFGLMADDEAAIIRILKDNQDAFLNQLERVDGSLEMGLRMSWDVPNIFEYFVGAHPELQELRDDFFRDGSNLTQDQMITLGRSFERLLEQDREEYTEQVESVMRSCCREIKRNKCRTEKEVLHLACLVDRDAAGRFEQVVLQAARPFDNNYAFDFNGPWAPHNFVEMDIHV

SEQ ID NO: 143 - protein, 244 aa, Tolypothrix sp.
  source 1..244: note = PCC 7601
MDAGLYLYGIFSDPIPPTVSLKGLDSQPVYSQVIEGFTFLYSDAKQEKYLASRRNLISHEKVLEQAMQEGFRTLLPLRFGLVVKNWETVISQLIQPCERQLRDLFQKLAGKREVSVKILWDTKAELQAMMQSNPDLKQKRDQMEGKNLSMEEVIEIGQLIESNLQQRKEAVIKTFFDELKPLAEEVVESEPMMEEMIYNAAFLIPWDQEALFSQRVEAIDKKFGDRLRIRYNNFTAPYTFAQIS

SEQ ID NO: 144 - protein, 266 aa, Trichodesmium erythraeum
  source 1..266: note = IMS101
MEFGFYVYGLIQEKGKMDESKDESKNGLKGSNESKDELKGLDKEDVKIQDVDEFAVLYSIAKKERYLASRRNLITHEKVLESAMEAGYRNLLPMQFGLVVSEWEKFSQDFTKPCEQQIHDLFTKLKNNREVGIKIYWEPDAELEKLLENDKDLKEERDSLKDKKLTMDQVIDIGQKIEQGMNERKQNIIEIFQETLNKMAIEVIENEVQTEKMIYNAAYLIPWDQEEDFGEKVETIDSKLCERGNFTIRYNSFTAPYNFARIRQQD

SEQ ID NO: 145 - protein, 334 aa, Ancylobacter aquaticus
  source 1..334: note = strain UV5
MTDLLVFAVVPADRFDPAILAEGDGLPPGLRAIAAGPLAAVVGAAPEGGLKGRERSALLPWLLASQKVMERLLANAPVLPVALGTVVEDEGRVRHMLDAGAAILGEGFQAVGDGIEMNLSVLWHLDTVVARLLPGVAPELRQAAAGGDAIERQALGVVLAGLVSAERRRARARVIEALQAVTRDFAIGEPTEPGGVVNLALLVDRAAEEALGAALEALDAEFDGALTFRLVGPLPPYSFASVQVHLSPAAAVCGARAALGVEPDASPETVKAAYRRAARETHPDLVPMGGEDEEAPEATADETSRFVVLSDAYRVLEGEHAPVSLRRLDSVLTE

SEQ ID NO: 146 - protein, 255 aa, Ancylobacter aquaticus
  source 1..255: note = strain UV5
MLYVYAITADYAAGANHLLPAKGIVPGVPVQRFGTGALGAVASPVPVTVFGKEALHALLDDADWTRARILAHQRVVSSLLPLATVLPLKFGTLVAGEASLAAALTSQHDALDATVARLRGAREWGVKLFFEAPTRTIRAEEPVGAGAGLAFFRRKKEEQETRAAAEAALDRCVAASHRRLASHARAAVANPLQPPELHGHPGTMGLNGAYLVAAENEAAWRVCFSELEQAYAALGARYVRTGPWAAYNFTGGGLV

SEQ ID NO: 147 - protein, 337 aa, Aquabacter spiritensis
  source 1..337: note = strain DSM 9035
MSGLLVFAIVPADRIEPGLLAPAEGLPPGLETVVAAGFAAIVGTAPEGGLKGRDRGSLLPWLLASQKVIERLMARGPVLPAALGSVLEDESRVRHMLVCGQAALAAAFETLNGCWQTDLSVRWDLSRTVAHLMTELPPGLRAAAETGDETARRSLGAALAGLVAGERRRIQSRIGAVLGAVARDLIVSDPVEPEGVVGVALLVDAPASAQVDAALDRLDGEFEGRLTFRLVGPLAPYSFATVQIHLGPAAGLAGAHAELGLEAGAPLEAVKAAYHRLIVGLHPDLVPHGSPGDDADDAASGKGGRAARFAAVTAAYRTLQAEHAPVSLRRQDGLSPG

SEQ ID NO: 148 - protein, 257 aa, Aquabacter spiritensis
  source 1..257: note = strain DSM 9035
MLYVYAITADHPGPHDAGSLPGEGIVPGAPVRLLPFGDLAAAVSPVSAVDFGPEALPARLQDVDWTGQRVLAHQRVVDSLVDVATVLPMKFCTLFSGAAALRAALADNRAALEATVVRLRGAREWGVKLFWEAPPAEPAPVERGPGAGAAFFQRKRDAQRLRAEAEAALAHGVAESHRRLAARARAAVANPVQPAAVHRRRGEMALNGAYLVPRADEAAWRESLAELERTYAGAGIRYELTGPWGPYNFTGGGLAGS

SEQ ID NO: 149 - protein, 334 aa, Bradyrhizobium oligotrophicum
  source 1..334: note = S58
MTMNLVGITTPDVAGAIAAAGGRLADVETRAVEAGGLVALLALSKAPFWHVLRRSRTALRSMLTAQRILEAAAVYGPLLPARPGTLIRNDAEACMLLRSQCRHLAEGLRLHGTSRQYQITISWDPVAALAARRDHQDLVEAAAASADGAADKAASMIQRFMSDQQARFEAEAMRALAAVAEDVITLPVNQPDMLMNAVVLLAPGAEPELERVLEALDRGLRGKNLIRLIGPLPPVSFAAVSIERPGRQRIAAARRLLGIGEATRTCDLRRAYLDKAHAHHPDTGGHAADASIVGAAAEAFRLLARVAEARASAGQDDVILVDIRRQDQQRSLST

SEQ ID NO: 150 - protein, 253 aa, Bradyrhizobium oligotrophicum
  source 1..253: note = S58
MSKANLGIGLVHGVVTAQSAALLPQIVDAFDATEIIVVNTEQQALLISDIPQYLRGHVEADTLFSDPARISTLAMKHHRILQAAAVVTDVVPVRLGTLVRGPSGARDLLNREAVRFAGHLVTIHNALEFSVRILPTEQPSRRVARPVPSSGRDYLRIRRDERCGQRPAVVDITLQELASRAVAIRERQSASRSGGRTPALAEAAFLVDRHALAAFDDCAGRIERQIAENGLALDIFGPWPAYSFVDGARENLG

SEQ ID NO: 151 - protein, 214 aa, Bradyrhizobium oligotrophicum
  source 1..214: note = S58
MSSPRLIGLLAADDVPADLADQIMSCGPVAAAIRFAPAAASSSESLDHHAAVVAWCRRAAFLPSRAGIPISPELLQSIARSAWYHRSTIEHIEGRVEISVELERRDGVRDGGIDGGGRAYLRATAHDLRACEVGVATAANLLAMYSERADADLIARTAPLPAIRLRASVLVRRAVAPRLARQFDSMLSAISDRLVCRVTGPWPPYSFSTIREPS

SEQ ID NO: 152 - protein, 252 aa, Burkholderia thailandensis
  source 1..252: note = sp. Bp5365 strain MSMB43
MVWLTYAVLTPKRSITLPPGVAGARLEIVDGAHLRTIVSEHPRAPSATIPSALDFGQTVAALFRHGAIVPMRFPTCLDSKQAVRDWLDDESDMYRDLLQRIDGCVEMGLRFRLPEAPRAQPRPQAGGPGHAYLAARGAPNSVARSHGERIAAVLRNLYRDWRFDGLVEGFVSLSFLVRQTTLDDFVDRCRQAARETAFPLYMSGPWPPYSFATDERSSAPEPHRALRLMRRPSTAVSISANVAAPEKKDSAR

SEQ ID NO: 153 - protein, 257 aa, Desulfobacterium vacuolatum
  source 1..257: note = DSM 3385
MTLHLLYCVFSSGEMEKTRKLVPPGIDGEPVHEICSNKISGVVSTLGKPPDTHVKSLLAYHGVIDSYHQNRTVIPMRFAAVFRTYAHMITALNNNEKSYLLQLKRLHDCTEMCVRFISNSPCCVKKKEPAISPKKISGTTFLQQRKAMYEQQNRLPPEIHEKTRDILQHFRGLYMEFKQESQPLEKDCPSLSLQGAEKTDGNALLISLFFLISKKNISLFRSRFQNICGSSSGRHMMNGPWPPFNFINTESNLTDPS

SEQ ID NO: 154 - protein, 265 aa, Desulfomonile tiedjei
  source 1..265: note = DSM 6799
MLGSLAAIQFLSISSYGADEMKFLMYCIFTENSIEPPHSLVGVNRSPVRIISCDGLAAAVSVITQKEIPRDPATGLDYHKVIQWFHERIGVIPLRLGTCLGHESDVVQLLHSHGARYKSLLKELDGCVEMGIRVIHDRPGPQELASKSPFISRFNGTESGTDYLMRRKVLFDADEFAISRNREIVERYHSPFTGLYVSFKAQTSKFSPLGTDRNSVLTSLYFLIPRQSADSFRAIYGDLRSGLHERIMLSGPWPPYNFVLPEDCL

SEQ ID NO: 155 - protein, 279 aa, Enhydrobacter aerosaccus
  source 1..279: note = strain ATCC 27094
MEGHRIYIYGIVRDAADGGPAPVPPVAGLDGGALRAIAGYGLAAIASAVDLSKAGIPFEEQLKDPDRATALVLEHHRVLQQAIDAQTVLPMRFGALFQDDRGVTDALEKNRCGLMDALGRIDGAREWGVKIFCDRAVAARQLSATSAVVQAAEKELSGLAEGRAFFLRRRLERLRTEETDRAVAHEVDVSRQALCELARASAPLKLQPAAVHGRGEDMVWNGAFLVPRSGEERFLSRLEVVVQSRSDLGLHYEVTGPWPPFSFVDGQLEGGGDACPDGA

SEQ ID NO: 156 - protein, 278 aa, Octadecabacter antarcticus
  source 1..278: note = 307
MRSATSIVYAYGVLTNCSDIALDMPRSDLAGLVKNGPLRILPFGNIAAVVCDFVLPNGSDLETLLEDSRSAERLILNHHQVLSYIVSQHTILPLRFGAAFTEDAGVIAALGGRCSELQKALGRIDGALEWGVKTFCDRKLLKQRVRGTGSEISDLESEIAKQGEGKAFFLRRRKERLILEEVEEILEQCVVGTQEQLEPSVIEEALVKLQPPTVHGHEHDMLSNISYLIARGTEDAFMQSLEDLRLAHAPYGLEYQMNGPWPAYSFSDQQLEGGVNDQ

SEQ ID NO: 157 - protein, 278 aa, Octadecabacter arcticus
  source 1..278: note = 238
MSSATSIVYVYGVLTNCSDLVLDFPPGDLAGIVESGPLRILPFGDIGALVCDFILPDGSDLKTILEDSRSAERMILNHHLVLADMVSRYTILPLRFGAVFAEDAGVIAALGGRYSTLQKELDRIDGAIEWGVKSFCNRKMFSECVAETVSEISVLEKEIADQGEGKAFFLRRRIQRLILDEVEKTLEQCLVGAQDQLKSRAIEETLVKLQPPTVHGHKHEMVSNRSYLIARGAEDAFMQSLDDLRVVYAPFGFDYQINGPWPAYSFSDQQLGGGVNDK

SEQ ID NO: 158 - protein, 261 aa, Rhodobacter capsulatus
  source 1..261: note = SB 1003
MGHYLYGLLAPPARGTLAQMQAAAAGVTSLGGPVALSAVEGMLLVHCPCDLAEISQTRRNMLAHTRMLEALMPLATCLPVRFGVIAQDLAEVARMIHERRAELVGHAQRLLDPVEIGLRVRFPRDRALAQLMAETPDFVAERDRLMGQGAGAHFARADFGRRLAEALDARRTRDQKRLLAALRPHVRDHVLRAPEEDVEVLRAEFLIPAAGVDAFSRIAHDLAAALGFAGAAEPELQVIGPAPPYHFLSLSLAFDNTSEAA

SEQ ID NO: 159 - protein, 316 aa, Rhodobacter capsulatus
  source 1..316: note = SB 1003
MAHEIIAILPCEAAQLPSGLTGVVGRGATAVLAPAPGWAERLTGGPKQTAVRHHSRLEALMAMGSVLPFAAGIACTPEEAALLLRLDAPLIARLAAEIGPRRHFQLALDWDESRVLAAFRDSPELAPLFSGAAVTPEALRQAITALADRLSATALRLLDPVAEDPVEQPRAPGCLLNLVFLLRPEDEPRLDAALQAIDALWSEGLRLRLIGPSAPISHALVDIDRADVAALAAAADLLKVAPEAGPEAVTEAAKAALRSPDLAANAAEQIRAAARLLLRAGDIAALGLSGAATLPHLVHLRPGGRKSGLTSSGEAA

SEQ ID NO: 160 - protein, 231 aa, Rhodobacter capsulatus
  source 1..231: note = SB 1003
MTGLALHGFVSPDGWSAAAAPPARCAVVLGGVAALVSEAGDALDTPETAQAAALAHHALISAWHRRGPVLPVRLGTVFSSQAALQTALAPKAAQLRAALDALADKEEMVLTIVPAARPPDLPPPAATGADWLRARKAVRDRGQARQTDRQQTLAGLQDALRAQGVASLAAPAPREGGSRWHLLIARDDGAGLDRWLAAQADRFDAAGLDLTLDGPWPPYRFAAEILEALDG

SEQ ID NO: 161 - protein, 220 aa, Rhodobacter capsulatus
  source 1..220: note = SB 1003
MSEPRISGLAPWRADLPDVIGCHGGWVLMGAAADETPEARLRRQVGWCRAAVDVLPLSPRLAPTRAEAERLVATRGPDLERAHRHIRGRLQVIVQLEMCRTDLGLVRREISGGRSWLQDRAERATREARANADFEAQVRRVVRALFPREGQVVTLAPSGTAGQLRLRRAVLVPRAGLQAFAAALSADLDRDGRGGLWDVIAPLPPLAFAALEAGPGGAVT

SEQ ID NO: 162 - protein, 254 aa, Rhodobacter sphaeroides
  source 1..254: note = 2.4.1
MIYLYGLLEEPASGHEVLAGMAGVTGPIALARLPGGILIYSSATEADILPRRRLLLAHTRVLEAAAWFGNLLPMRFGMMASTLAEVAAMLASRLTELCAAFDRVRGRVELGLRLSFPREPALAATLATAPDLAAERARLLALRRPDPMAQAEFGRRLAERLDARRGETQRLLFQSLRPLWVDHRLRVPDSDVQVIAVDVLVEDGAQDRLAAALVKAAADCSFAPTAEPSVRVIGPVPLFNFVDLVLSPRREEVA

SEQ ID NO: 163 - protein, 291 aa, Rhodobacter sphaeroides
  source 1..291: note = 2.4.1
MRLREVVAVLEGHPPSVLPEGTEAICEAGLTAILGMPPGLLSGRRALLEHAACRQAVLERLMAFGTVLPVLTGNCLTPAEAAAALAANSPRLRQELRRLAGRVQFQVLVQWHAALVPKRTDPDETAEDLRLRFTHRIADALARVAERHVNLPLREDMLANQALLLLQTRTDDLDRSLEQIDALWTEGLRIRRIGPSPPVSFASLNFRRVSSAAIRRARHRFDLEGPVDPIRLRALRRDLLLRASEAERAEILAAAAVLDLLTRCAASGGDLHLVRIWSEGQAVPSDLEDAA

SEQ ID NO: 164 - protein, 232 aa, Rhodobacter sphaeroides
  source 1..232: note = 2.4.1
MSGLLLLGVVSGLGISPAITSPHLRLDGDGYAAILLSLDRLPPDPASPDWAVQAALAQNAILSAYAATEDVLPVALGAAFTGIAAVKRHLDAERATLDAGMERLAGRAEYVAQLIAEQVADGAAPAPASGSAFLKARSARHEQRRHLARERTGFARATAEELASLSCSASARPLKPDGPLLDLSLLVARDRVPGLLEAAEASSRAGSRLALSVRLIGPCAPFSFLPETRGHD

SEQ ID NO: 165 - protein, 169 aa, Rhodobacter sphaeroides
  source 1..169: note = 2.4.1
MAGDARSRVRLHLAAMRDCETFLPFPPAATIAVDEAIAWCGRRTNALAEEIDRFSRQRQLTVSARLIAPLLPDAAASGAGWLRARRDASAHQARLRTVLMQIMSLLGEVRCIPGRLQDEVQVNLLVPAAETHPVLHELRERLRVGDALWSACTVTGPWPPYAFISWETA

SEQ ID NO: 166 - protein, 260 aa, Rhodococcus hoagie
  source 1..260: note = 103S
MSEQESAPDGGGPVVYVYGLVPADVEVKEDATGIGSPPRPLKIVHHEDVAALVSEIDPDTPLGSSDDLRAHAAVLDSTATVAPVLPLRFGAVLTDTDAVVAELLEPYRDEFHEALEQLEGKVEFVVKGKYVEDAILREILADDPEAARLRDVVREQPEDTTRDERLALGERISQALTAKREQDTGRIVEALQPAATAVAPREPTDDEEAGSVAVLISADGVDELDKAVARLIDDWQGRVEVTVTGPLAAYDFVKTRAPGT

SEQ ID NO: 167 - protein, 259 aa, Rhodococcus hoagie
  source 1..259: note = 103S
MTPDDGVWVYAVTGDGSFPGGISGIRGVAGEELRTVTDSGFTAVVGTVRLDTFGEEALRRNLEDLDWLADTARRHDAVVAAICAGGATVPLRLATVYFDDDRVRTMLRDNAEQLGEALQQIADRSEWGVRAYLERPRSEPRDAREKTGRPSGTAYLMQRRAQVAAREQAESAAGRRADEIFAELARWAVAGVRQPPSPPDLAGRRSQEILNTSFLVDNGRHREFVTAVEELDARLSDVDLVLTGPWPPYSFTSVEASAR

SEQ ID NO: 168 - protein, 222 aa, Serratia sp
  source 1..222: note = ATCC 39006
MSLLLYGIVAEDTQLALEPDGSPHAGEEPMQLVKAATLAALVKPCEADVSREPAAALAFGQQIMHVHQQTTIIPIRYGCVLADEDAVTQHLLNHEAHYQTQLVELENCDEMGIRLSLASAEDNAVTTPQASGLDYLRSRKLAYAVPEHAERQAALLNNAFTGLYRRHCAEISMFNGQRTYLLSYLVPRTGLQAFRDQFNTLANNMTDIGVISGPWPPYNFAS

SEQ ID NO: 169 - protein, 362 aa, Stella vacuolate
  source 1..362: note = ATCC-43931
MSGLLVFAIVPADGIEPGILAPREELPANLRAVAADGFAAVVGAAPEGGLKGRDRSVLLPRLLASQKVIERLMARGPVLPVTLGTVLEDEARVRHMLAAGAPMLEAAFGTLGDCWQMDLSVRWDLNQVVARLMGEVPGDVRAAAGSGDEAARRALGEALAGLAAGERRRVQSRLAAALRDVARDLIVSEPVEPESVVDIAILVERPALAEVEAALDRLDAEFEGRLKFRLVGPLAPHSFATVQVHLAPEAALAGACAELGVERGAGLQDVKVAYHRALVRFHPDLAPHGDDGGPEDEHDGGEGRASRLLTVTAAYRALQAEHAPISLRRQDGIAVNQEQDASAAMGQQRGIVPGRELQALRM

SEQ ID NO: 170 - protein, 258 aa, Stella vacuolate
  source 1..258: note = ATCC-43931
MLYVYAIAADHPDPDNAMFGGEGIVPDAPVRLLQLGDLAVAASLVSAADFAADALRAHLEDARWTALRVLAHQRVVDSLLPHATVLPMKFCTLFSGEAALKQALAHNRAALQATVERLRGAREWGVKLYWEAPRNPAPPSAGQGEAGAGAAFFQRKRDQQRQRAEAEAAVARCVAASHRRLADAARAAVANPVQPPAVHRQPGEMALNGAYLVARAAEPAWREVLAELERTHADGGIRYELTGPWGPYNFTGSGLVGS

SEQ ID NO: 171 - protein, 253 aa, Thiocapsa rosea
  source 1..253: note = strain DSM 235
MSDRPRPMLHCILRSPPGSIARAEAGLRWIERDGLAALVADREPSEIAGASSVGLQRYADIVAEIHACAAVIPVRFGCLLAGDEAVGKLLHRSRDRLHGLLDQVGDCLEFGIRLLLPADAPAATDDDAAPRLHANAPSDPRADPDMGPGLSHLLAIRHRLDVEASLAARAREAREVIKGRVAGRFREVREELGQIDGRSLLSLYFLVPREQGEHFVECLRQDASSLRGTGLLTGPWPPYNFVGAIDDDIRSLD

SEQ ID NO: 172 - protein, 119 aa, Anabaena-flos-aquae
MLTKLLLLPIMGPLNGVVWIAEQIQERTNTEFDAQENLHKQLLSLQLSFDIGEIGEEEFEIQEEEILLKIQALEEEARLELEAEQEEARLELEAEQEDFEYPPQFTAEVNKDQHLVLLP

SEQ ID NO: 173 - protein, 88 aa, Bacillus-megaterium
VLHKLVTAPINLVVKIGEKVQEEADKQLYDLPTIQQKLIQLQMMFELGEIPEEAFQEKEDELLMRYEIAKRREIEQWEELTQKRNEES

SEQ ID NO: 174 - protein, 83 aa, Ancylobacter aquaticus
  source 1..83: note = strain UV5
MGMLTDVVFAPAVGPLKGVLWLARIIAEQAERTLYDEGVIRAALLDLEQQLEAGEIDEDAYETQETVLLERLKIARERMRSGL

SEQ ID NO: 175 - protein, 122 aa, Aphanizomenon flos-aquae
  source 1..122: note = NIES-81
MLTKLLLLPIMGPLNGLVWIGEQIQERTNTEFDAQENLHKQLLNLQLSFDIGEISEEDFEIQEEELLLKIQALEEEARLELELAEEEARLELELEQEEEEDFVVKPQLTTEIDRDKDLVLLP

SEQ ID NO: 176 - protein, 86 aa, Aphanothece halophytica
  source 1..86: note = strain PCC 7418
MVFKLLLLPITGPIEGVTWLGEQILERANQELDEKENLNKRLLSLQLSLDLGEISEEEYDEQEEEILLAMQAMEDEENNQAEEETD

SEQ ID NO: 177 - protein, 83 aa, Aquabacter spiritensis
  source 1..83: note = strain DSM 9035
MSLVTDVLFAPAVGPLKGVLWLARLIAEQAERTLYDEDVLRAALLDLEQRFEAGEISEADYETEEDILLARLKIARERMRSGL

SEQ ID NO: 178 - protein, 82 aa, Bradyrhizobium oligotrophicum
  source 1..82: note = S58
MLFQILTSPVSGPFRMVSWIGGAIRDAVDTKMNDPAEIKRALAALEQQLEAGSLSEQDYERMEMELIERLQSSLRHGSGNGG

SEQ ID NO: 179 - protein, 75 aa, Burkholderia thailandensis
  source 1..75: note = sp. Bp5365 strain MSMB43
MFILDNLLAAPIKGMFWIFEEIAQAAEEETIADIEMIKAALVELYRELESGQIDETEFETRERALLDRLDSLETS

SEQ ID NO: 180 - protein, 78 aa, Chlorobium luteolum
  source 1..78: note = DSM 273
MFILDDILLAPLSGMVFLGRKINEIVQNEMSDEGAVKEQLMKLQFRFEMDELSEEEYDRLEDELLSTLAEIRAQKENR

SEQ ID NO: 181 - protein, 83 aa, Dactylococcopsis salina
  source 1..83: note = PCC 8305
MVFKLLLLPITGPIEGITWLGEQILERADQELDSKENLNKRLLSLQLSLDLGEISEEEYDEQEEEILLAMQAMEDEENEEEES

SEQ ID NO: 182 - protein, 77 aa, Desulfobacterium vacuolatum
  source 1..77: note = DSM 3385
MFLVDDILFFPAKSLVWVFRELHNAVQQEKTNESDALTTELSELYMMLETGKITEEEFDEREEQILDRLDEIQERDQ

SEQ ID NO: 183 - protein, 86 aa, Desulfomonile tiedjei
  source 1..86: note = DSM 6799
MERYTMFLLDDILFLPMNGVLWICNEIHDAAEQELHNESDAITAQLQKLYTLLEAGDIGESEFDVLEAELLDRLDAIQERGALLEA

SEQ ID NO: 184 - protein, 83 aa, Desulfotomaculum acetoxidans
  source 1..83: note = DSM 771
MLGKLLLSPILGPVMGVKFIAEKIKQQADQELYDKSKIKQDLMELQIKLELEEITEEYYLQREEELLVRLDELASMETEEEEV

SEQ ID NO: 185 - protein, 129 aa, Dolichospermum circinale
MLTQLLLLPIMGPLNGVVWIAEQIQERTNTEFDAQENLHKQLLSLQLSFDIGEISEEEFEIQEEEILLKIQALEEEARLELEAEQEEARLELEAEQEQARLELEAEQEELENQPQLTPKIDTYRHLVKL

SEQ ID NO: 186 - protein, 81 aa, Enhydrobacter aerosaccus
  source 1..81: note = strain ATCC 27094
MGMLARLLTLPVSAPVGGVLWIARKIEEEANAERWDRNKITGALSELELELDLGAIDVEEYDAREAVLLQKLKELQEVEND

SEQ ID NO: 187 - protein, 103 aa, Isosphaera pallida
  source 1..103: note = ATCC-43644
MFLVDDILLAPAHSLMFLLREIHQAALEELRRDAQKVREELAECYRALETGALTDEEFASLETDLLDRLDALEELARFNSDEDDDPEDEDWDVEDDDPAEAVW

SEQ ID NO: 188 - protein, 77 aa, Legionella drancourtii
  source 1..77: note = LLAP12
MLLLGSILMAPVHGLMAIFEKIKEAVDEEKQHDIERIKSELMALYTKLESGELSEADFEKQEKILLDKLDSLEDEDD

SEQ ID NO: 189 - protein, 86 aa, Microcystis aeruginosa
  source 1..86: note = NIES-843
MFLDLLFLPVTGPIGGLIWIGEKIQERADIEYDEAENLHKLLLSLQLSYDMGNISEEEFEIQEEELLLKIQALEEEEAENESESSL

SEQ ID NO: 190 - protein, 84 aa, Nostoc punctiforme
  source 1..84: note = ATCC 29133
MVLRFLLLPITGPLMGVTWLGEKILEQASTEIDDKENLSKQLLALQLAFDMGEIPEEEFEIQEEALLLAILEAEQEERDQTQEY

SEQ ID NO: 191 - protein, 125 aa, Nostoc sp
  source 1..125: note = PCC 7120
MLGKILLLPVMGPINGLMWIGEQIQERTNTEFDAQENLHKQLLSLQLKFDMGEISEEEFDIQEEEILLKIQALEAEERLNAESEEDDDLDVQPIFILASEENPVYQDQSRFSEEYEDKEDLVLSP

SEQ ID NO: 192 - protein, 84 aa, Octadecabacter antarcticus
  source 1..84: note = 307
MGIILNTLMSPLIGPMKGVFWVAEQIKDQTDAEIYDDSKILVELSELELLLDLEKIELKDFEAKEDVLLKRLQEIRKAKKNDSV

SEQ ID NO: 193 - protein, 84 aa, Octadecabacter arcticus
  source 1..84: note = 238
MSIILNTLMGPLIGPMKGLLWVAEQIKDQADAELYDDSKILVALSELELSFDLEQIELKEFEAQEDVLLQRLQAIRKAKQNDTD

SEQ ID NO: 194 - protein, 78 aa, Pelodictyon phaeoclathratiforme
MFILDDILFAPLNGLIFIAKKINDVVEKETSDEGVVKERLMALQLRFELDEIDEVEYDREEDELLQKLERIRLNKQNQ

SEQ ID NO: 195 - protein, 81 aa, Phormidium tenue
  source 1..81: note = NIES-30
MLFKLLFAPVLGPIEGISWVANKLLEQADVPTNDLESLQKQLLALQLAFDMGEVAEADFEIQEEEILLAIQAIEDEEDEDE

SEQ ID NO: 196 - protein, 85 aa, Planktothrix agardhii
  source 1..85: note = str. 7805
MILRLLLSPITAPFEGVIWIGEQLLERAEAELDDKENLGKRLLALQLAFDMGDIPEEDFEVQEEELLLQIQALEDEANQENDEID

SEQ ID NO: 197 - protein, 149 aa, Psychromonas ingrahamii
  source 1..149: note = 37
MFILDDILLAPYSGIKWLFKEIQRQAQEELDGEADRITTDLTNLYRQFESNEITEQEFEERETVLLDRLDELQEESNLLDEEYDEEYEDDDEEYEDDDEEYEDDDEEYEDDDEEYEDDDKNDKDKNDDHDNDDDDENKDENDKYNDEER

SEQ ID NO: 198 - protein, 85 aa, Rhodobacter capsulatus
  source 1..85: note = SB 1003
MGLLRKLLLAPVELPITGALWIVEKIAETAESELTDPGTVRRLLRGLEQQLEAGEITEEEYEFAEEILLDRLKRGQAAEARSGGP

SEQ ID NO: 199 - protein, 77 aa, Rhodobacter sphaeroides
  source 1..77: note = 2.4.1
MGLLTSLLTLPFRGPFDGTLWIAARIGEAAEQSWNDPAALRAALVEAERQLLAGELSEETYDAIELDLLERLKGTAR

SEQ ID NO: 200 - protein, 83 aa, Rhodococcus hoagie
  source 1..83: note = 103S
MGLFSAIFGLPLAPVRGVVWIGEVVRRQVEEETTSPAAMRRDLEAIEEGRRSGEISEDEAAQAEDEILHRVTRRRDAGASGEE

SEQ ID NO: 201 - protein, 138 aa, Serratia sp
  source 1..138: note = ATCC 39006
MLLIDDILFSPVKGVMWIFRQIHELAEDELAGEADRIRESLTDLYMLLETGQITEDEFEQQEAVLLDRLDALDEEDDMLGDEPGDDEDDEYEEDDDEEDDDEEDDDDEDDDDEDDDDEEDDDDDEDDDDEDEPEGTTK

SEQ ID NO: 202 - protein, 83 aa, Stella vacuolate
  source 1..83: note = ATCC-43931
MGLVTNVAFAPVVGPLKGVLWLARLIADQAERTLYDEDLVRAALLDLEQRLDAGQISEADYDAEEEILLARLKIARERMRSGL

SEQ ID NO: 203 - protein, 98 aa, Thiocapsa rosea
  source 1..98: note = strain DSM 235
MLIVDDLLAAPFKGIIWVFEEIHKSATAEQRARRDEIMAALSALYRALEQGEITDDTFDTREQALLDELDALDAREDANELGSDEDEDDLDGAGEDAS

SEQ ID NO: 204 - protein, 132 aa, Tolypothrix sp
  source 1..132: note = PCC 7601
MEVMIMLGKILLFPVMGPISGLMWIGEQIQERTDTEFDAQENLHKQLLSLQLSFDIGEISEEDFEEQEEELLLKIQALEEEKARLEAESIEDEEDEVEPTYFIAEVEEDKVLAEAFRGNKKYEDNENLVLSP

SEQ ID NO: 205 - protein, 85 aa, Trichodesmium erythraeum
  source 1..85: note = IMS101
MLLRLLTLPISGPLEGVTWLGKKLQEQVDTEIDETENLSKKLLTLQLAFDMGEISEEDFEDQEEELLLAIQALEEQKLKEEEEDA

SEQ ID NO: 206 - protein, 248 aa, Anabaena-flos-aquae
MLPTRPQTNSSRTINTSTQGSTLADILERVLDKGIVIAGDISISIASTELVHIRIRLLISSVDKAKEMGINWWESDPYLSTKAQRLVEENQQLQHRLESLEAKLNSLTSSSVKEEIPLAADVKDDLYQTSAKIPSPVDTPIEVLDFQAQSSGGTPPYVNTSMEILDFQAQTSAESSSPVGSTVEILDFQAQTSEESSSPVVSTVEILDFQAQTSEESSSPVGSTVEILDFQAQTSEEIPSSVDPAIDV

SEQ ID NO: 207 - protein, 100 aa, Bacillus-megaterium
MAVEHNMQSSTIVDVLEKILDKGVVIAGDITVGIADVELLTIKIRLIVASVDKAKEIGMDWWENDPYLSSKGANNKALEEENKMLHERLKTLEEKIETKR

SEQ ID NO: 208 - protein, 120 aa, Ancylobacter aquaticus
  source 1..120: note = strain UV5
MNEQRMEHSLQAVGLADILERVLDKGIVIAGDITISLVEVELLNIRLRLVVASVDRAMSMGINWWQSDPHLNSHARELAEENKLLRERLDRLEAAVVPSALPADAALEPSLAGEDARHGG

SEQ ID NO: 209 - protein, 72 aa, Ancylobacter aquaticus
  source 1..72: note = strain UV5
MPSRHSGEIAVADLLDRALHKGLVVWGEATISVAGVDLVYLGLKLLLTSTDTVNRMREAANAPPDERHLHAD

SEQ ID NO: 210 - protein, 157 aa, Aphanizomenon flos-aquae
  source 1..157: note = NIES-81
VTSTPILPTRPQTNSSRAINTSTQGSTLADILERVLDKGIVIAGDISISIASTELIHIRIRLLIASVDKAKEMGINWWETDPYLSTKAQRLVEENQQLQNRLENLESQINLLTSAKVQEQISLVETTEDNTHQTTEDNTHQTHEESIPLPIDSQLDV

SEQ ID NO: 211 - protein, 128 aa, Aphanothece halophytica
  source 1..128: note = strain PCC 7418
MVNPNTNKPKSYQSKGITNSTQSSSLADILERVLDKGIVIAGDITVSVGSTELLSIRIRLLVSSVDKARELGINWWEGDPYLSSQANLLKEENQALQNRLENMEAELRRLKGETNPEPSFLSESEDNS

SEQ ID NO: 212 - protein, 114 aa, Aquabacter spiritensis
  source 1..114: note = strain DSM 9035
MSEQRMEHSLQAVGLADILERVLDKGIVIAGDISISLVEVDLLNIRLRLVVASVDRAMSMGINWWQSDPHLNSHARQLEEENRLLRERLDRLEAALAPPEGGMLRAEVEVAHGG

SEQ ID NO: 213 - protein, 78 aa, Aquabacter spiritensis
  source 1..78: note = strain DSM 9035
MPDPEPIIPRTSGDVALADLLDRALHKGLVLWGEATISVAGVDLVYLGLKVLLASTDTANRMRDAAAASAAGSHLPGG

SEQ ID NO: 214 - protein, 127 aa, Arthrospira platensis
  source 1..127: note = NIES-39
MTLQSRSSSPQRGVPMSTSGSSLADILERVLDKGIVIAGDISVSVGSTELLSIRIRLLIASVDKAKEIGINWWESDPYLSSQAQQLSQSNQQLLEEVKRLQEEVRSLKALTSQSSQPVTPPNSENDD

SEQ ID NO: 215 - protein, 119 aa, Bradyrhizobium oligotrophicum
  source 1..119: note = S58
MTFTVHQPTGGDRLADILERVLDKGIVVAGDVTISLVGIELLNIKIRLIVATVDRALELGINWWEADPRLTTRASELSVENEELKKRLALLEADAGRNQRPRKRRVRSIAATSGASHER

SEQ ID NO: 216 - protein, 81 aa, Bradyrhizobium oligotrophicum
  source 1..81: note = S58
MTYRADLDYLEPAASSEGSLLELLDHLLDRGVLLWGELRISVADVELIEVGLKLMLASARTADRWRQTTTQRASIAPGDCP

SEQ ID NO: 217 - protein, 74 aa, Burkholderia thailandensis
  source 1..74: note = sp. Bp5365 strain MSMB43
MRSADGEPVSAELAQRLSLCESLDRILNKGAVISAQVVVSVADVDLLYLHLRLLLTSVETALVGRAMPREEASR

SEQ ID NO: 218 - protein, 89 aa, Burkholderia thailandensis
  source 1..89: note = sp. Bp5365 strain MSMB43
MADLLERVLDKGVVITGDIRINLVDVELLTIRIRLLVCSVDKAKELGIDWWNADTFFLGPDRGQSALPGRASAVDVAAGSAVHADAAHR

SEQ ID NO: 219 - protein, 97 aa, Chlorobium luteolum
  source 1..97: note = DSM 273
MPELKHAVNATGLADILERVLDKGIVIAGDIKIQIADIDLLTIKIRLMVASVDKAIEMGINWWQEDPYLSTGAKTSEQTRLLGEINQRIEKLESINR

SEQ ID NO: 220 - protein, 69 aa, Chlorobium luteolum
  source 1..69: note = DSM 273
MQEDLYTANRQVTLLDILDRVLNKGVVISGDIIISVAGIDLVYVGLRVLLSSVETMERLDAARAEGLQQ

SEQ ID NO: 221 - protein, 68 aa, Chlorobium luteolum; identical to SEQ ID NO: 102
  source 1..68: note = DSM 273

SEQ ID NO: 222 - protein, 68 aa, Chlorobium luteolum; identical to SEQ ID NO: 102
  source 1..68: note = DSM 273

SEQ ID NO: 223 - protein, 130 aa, Dactylococcopsis salina
  source 1..130: note = PCC 8305
MVNSNTNQPKSYQSKGITNSTQSSSLADILERVLDKGIVIAGDISVSVGSTELLTIRIRLLISSVDRAREIGINWWESDPYLSSQAHLMKEENQALQSRLENMEAELRRLKGETNLDQSSLGESDQRSLQ

SEQ ID NO: 224 - protein, 74 aa, Desulfobacterium vacuolatum
  source 1..74: note = DSM 3385
MAYIDIDNDASKQISICEALDRVLNKGAVITGELTISVADIDLIYLSLQAVLTSVETARHMFDSQINDAVKEVK

SEQ ID NO: 225 - protein, 105 aa, Desulfobacterium vacuolatum
  source 1..105: note = DSM 3385
MPIQRTAQHSIESTNIADLLERVLDKGIVIAGDIKISLVDIELLSIQLRLVICSVDKAKEMGMDWWVNNPVFMPNKGTQNDEIADTLTKINSRLEHLEKATISGS

SEQ ID NO: 226 - protein, 73 aa, Desulfomonile tiedjei
  source 1..73: note = DSM 6799
MMDEEEHVSLCEALDRVLNKGAVIAGEVTISVANVDLIYLGLQVVLASVDTIRGKRNELLRHDVGLHLTADNA

SEQ ID NO: 227 - protein, 81 aa, Desulfomonile tiedjei
  source 1..81: note = DSM 6799
MSIQASTRHSIQSTNLADLLERVLDKGVVIAGDIKIKLVDVELLTIQIRLVVCSVDKAKEMGMDWWTNNPAFQPALAQISE

SEQ ID NO: 228 - protein, 123 aa, Desulfotomaculum acetoxidans
  source 1..123: note = DSM 771
MGPQMGPIKSTGNLSLLDVIDRILDKGLVINADISVSIVGVELLGIKIKAAVASFETAAKYGLQFPTGTEINEKVSEAAKQLKEICPECGKKSGRDELLHEGCPWCGWISARALRLETEHSQR

SEQ ID NO: 229 - protein, 78 aa, Desulfotomaculum acetoxidans
  source 1..78: note = DSM 771
MLPIREERATLTDLLDRVLDKGLLLNADILISVAGVPLIGITLKAAIAGMETMKKYGLLIDWDQESRLAERRLRSSRH

SEQ ID NO: 230 - protein, 108 aa, Enhydrobacter aerosaccus
  source 1..108: note = strain ATCC 27094
MAVTNGRMEHSIQGSSLADILDRILDKGIVIAGDVTISLVGVELLNIRLRLLVASVDKAIEMGINWWEADPYLTSQTKASSEQTELLQQRLERIEGLLAGQATKEQPL

SEQ ID NO: 231 - protein, 74 aa, Enhydrobacter aerosaccus
  source 1..74: note = strain ATCC 27094
MPVQTAHDGELALADLLDRALNKGVVLWGDATISLAGVELVYVGLRVLVASCSTMEKYRSSPRKGSMPIARGES

SEQ ID NO: 232 - protein, 98 aa, Isosphaera pallida
  source 1..98: note = ATCC-43644
MIVCSSSTPERIGPPMNLPPPHHAPWCYDSPDLETLPLDPAERIALCEVLDRVLNKGVVIHGEITISVAGVDLVYLGLNLLLTSVETAQSWKFRGMIE

SEQ ID NO: 233 - protein, 171 aa, Isosphaera pallida
  source 1..171: note = ATCC-43644
MAITRSSRPDVTHSTSGATLADVLERVLDKGLVIAGDIKIKLVDVELLTIQIRLVVASVDKAREMGLDWWTRSPELSSLAATTCPALTPPKQEATPPATRIQAPTESAQTTPDQSHPSDPSASNIDEVAELRRHIELMQLRDEARQRAHREELAALRAQLTRLTELLDSPR

SEQ ID NO: 234 - protein, 61 aa, Legionella drancourtii
  source 1..61: note = LLAP12
MIIEDKPVSLCETLDRVLNKGVVVAGTVTISVADVDLLYLDLHCLLSSMKGMNLIGSERER

SEQ ID NO: 235 - protein, 106 aa, Legionella drancourtii
  source 1..106: note = LLAP12
MELQKSPTHSIGSTTIADLLERILDKGIVIAGDIKVNLVQVELLTIQIRLLICSVDKAKEIGMDWWTHQNDVQSKNGSMPIQEYVTQMEERLKNLENTLASSKNAI

SEQ ID NO: 236 - protein, 114 aa, Lyngbya confervoides
  source 1..114: note = BDU141951
MTGQSLSRSSSANRQMATATQGSTLVDVLERVLDKGIVIAGDISVSVGSTELLTIRIRLLVASVDKAREMGINWWENDPYLSARSQELLTANEQLQSRIESLEQELKSLRSQED

SEQ ID NO: 237 - protein, 136 aa, Microcystis aeruginosa
  source 1..136: note = NIES-843
MTSSTFAGSLRNQSNNSLKTATQGSSLADILERVLDKGIVIAGDISVSIASTELINIRIRLLIASVDKAREMGINWWEGDPYLHSQSQALLAENRELSLRLQTLETELETLKSLTQLSAMESHDTSPNDEAHSSDA

SEQ ID NO: 238 - protein, 116 aa, Nostoc punctiforme
  source 1..116: note = ATCC 29133
MSTNTNRGAITTSTQGSTLADILERVLDKGIVIAGDISISVGSTELLNIRIRLLISSVDKAKEIGINWWESDPYLNSQTRTLLATNQQLQERLASLETELQSLKALNPINHQNAGD

SEQ ID NO: 239 - protein, 148 aa, Nostoc sp
  source 1..148: note = PCC 7120
MTTTPIHPTRPQTNSNRVIPTSTQGSTLADILERVLDKGIVIAGDISISIASTELIHIRIRLLISSVDKAREMGINWWENDPYLSSKSQRLVEENQQLQQRLESLETQLRLLTSAAKEETTLTANNPEDLQPMYEVNSQEGDNSQLEA

SEQ ID NO: 240 - protein, 112 aa, Octadecabacter antarcticus
  source 1..112: note = 307
MNDGKMEHSLNATNLADILERVLDKGIVIAGDVTISLVGVELLNIKLRLLIASVDKAMEMGINWWAHDPFLTAGAQAPAVADPAMLERMDRLEAALATALASNQTTPMKGHK

SEQ ID NO: 241 - protein, 74 aa, Octadecabacter antarcticus
  source 1..74: note = 307
MTNKAQGGQDLALADLLDRALSTGVVIWGEATISLAGVDLVYVGLKVLVASVDAAERMKAASLVDRPTDRGQQI

SEQ ID NO: 242 - protein, 113 aa, Octadecabacter arcticus
  source 1..113: note = 238
MNNGKMEHSLDATNLADILERVLDKGIVIAGDVTISLVGVELLNIKLRLLIASVDKAMEMGINWWAHDPYLTAGAQAPVGVDPAMLERMDRLEAALAKALASNQTTPAEGQSS

SEQ ID NO: 243 - protein, 72 aa, Octadecabacter arcticus
  source 1..72: note = 238
MTNETQGGQDLALADLLDRALSTGVVIWGEATISLAGVDLVYVGLKVLVASVDAAQRMKDASLVDRPTDGGQ

SEQ ID NO: 244 - protein, 99 aa, Pelodictyon phaeoclathratiforme
MPELKHAVNATGLADILERVLDKGIVIAGDIKIQIADIDLLTIKIRLLIASVDKAMEMGINWWQEDTYLSTKAKDKEQQLLRDDLQQRIEKLEALTKIT

SEQ ID NO: 245 - protein, 66 aa, Pelodictyon phaeoclathratiforme
MQDEFYSKNKEITILDVLDRVLTKGVVITGDIVISVADIDLVYVGLRLLLSSVETMEKNKQNSIKM

SEQ ID NO: 246 - protein, 101 aa, Phormidium tenue
  source 1..101: note = NIES-30
MATATQGSSLVDVIERVLDKGIVIAGDISVSVGSTELLSIRIRLIISSVDKAREIGINWWESDPYLSSRTNELLEANQQLQSRLETLEAELKALRSAEPVS

SEQ ID NO: 247 - protein, 125 aa, Planktothrix agardhii
  source 1..125: note = str. 7805
MNSQQLPSNIQRGVPTSTQGSSLADILERVLDKGIVIAGDISVSVGSTELLNIRIRLLIASVDKAREIGINWWESDPYLSSQTKVLTESNQQLLEQVKFLQEEVKALKALLPQENQPNPISDPHK

SEQ ID NO: 248 - protein, 121 aa, Planktothrix rubescens
MNSQQRPSNIQRGVPTSTQGSSLADILERVLDKGIVIAGDISVSVGSTELLNIRIRLLIASVDKAREIGINWWESDPYLSSQTKVLTESNQELLEQVKLLQEEVKALKALLPQENQPKEME

SEQ ID NO: 249 - protein, 72 aa, Psychromonas ingrahamii; identical to SEQ ID NO: 94
  source 1..72: note = 37

SEQ ID NO: 250 - protein, 74 aa, Psychromonas ingrahamii
  source 1..74: note = 37
MPMANVSINPELTAQECEKISLCDALDRIINKGVVIHGEITISVANVDLISLGVRLILSNVETREQSNTPKEEV

SEQ ID NO: 251 - protein, 101 aa, Psychromonas ingrahamii
  source 1..101: note = 37
MATGKPQSMTHSVKSTTVADLLERILDKGIVVTGDIKIKLVDVELLTVELRLVICSVDKAVEMGMDWWNNNPAFAPQAPAQEGELSSIEKRLEKIEKALVK

SEQ ID NO: 252 - protein, 151 aa, Rhodobacter capsulatus
  source 1..151: note = SB 1003
MGYRSASQPEGLADVLERILDKGIVIAGDVSVSLVGIELLTIRLRLLIATVDKAREMGIDWWSHDPYLNGRLRPGEPAPETETETAALRDRLAQLEAQLSALGAQVGAAPALAEPALRGLAAAGSSALCAAPEASSADVVQPVFRRYKEAP

SEQ ID NO: 253 - protein, 69 aa, Rhodobacter capsulatus
  source 1..69: note = SB 1003
MDDRFSLRLFGPEEVFDAPSGGLADLLDGLLGHGIVLHGDLWLTVADVELVYVGLSAVLASPEALRSHE

SEQ ID NO: 254 - protein, 104 aa, Rhodobacter sphaeroides
  source 1..104: note = 2.4.1
MSFQMQSPLQQDSLADVLERILDKGIVIAGDISISLVGIELLTIRLRLLVATVDKAREMGINWWESDPRLCITQAPASDGSAALLDRLERIETQIGQLAAAREG

SEQ ID NO: 255 - protein, 74 aa, Rhodobacter sphaeroides
  source 1..74: note = 2.4.1
MTDSAPTLQFATAEEALQSSETRLVDVVDALLSQGIAIRGELWLTIADVDLVFLGLDLLLANPDRLQCRVPDAA

SEQ ID NO: 256 - protein, 137 aa, Rhodococcus hoagie
  source 1..137: note = 103S
MTRSGSGANYPQQYSQGLGGAGHEPANLGDILERVLDKGIVIAGDIRVNLLDIELLTIKLRLVIASLETAREVGIDWWEHDPWLSGNNRDLELENERLRARIEALESGERRVADVTDPHRAVQPAESPAAEVRDDDA

SEQ ID NO: 257 - protein, 65 aa, Serratia sp
  source 1..65: note = ATCC 39006
MPVNKQYQDEQQQVSLCEALDRVLNKGVVIVADITISVANIDLIYLSLQALVSSVEAKNRLPGRE

SEQ ID NO: 258 - protein, 110 aa, Serratia sp
  source 1..110: note = ATCC 39006
MSGNKKLTHSTDSTTVADLLERLLDKGVVISGDIRIRLVEVELLTLEIRLLICSVDKAVEMGLDWWSGNPAFDSRARVSSSAPAPELEERLQRLEARLEAAPSVIEETHL

SEQ ID NO: 259 - protein, 123 aa, Stella vacuolate
  source 1..123: note = ATCC-43931
MSGQRMEHSVQAVGLADILERVLDKGIVIAGDISISLVEVELLTIRLRLVVASVDRAMSMGINWWQSDPNLNSHARQLEEDNRLLRERLDRLEAALALPEMAGERLADAGQGGGAEQGVTHGR

SEQ ID NO: 260 - protein, 78 aa, Stella vacuolate
  source 1..78: note = ATCC-43931
MSDPEPIIPRTSGDIALADLLDRALHKGLVLWGEATISVAGVDLVYLGLKVLVASTETADRMRAAAASQSADPKVRAG

SEQ ID NO: 261 - protein, 71 aa, Thiocapsa rosea
  source 1..71: note = strain DSM 235
MMLAIGEHPDCPEEIQRVSLCEALDRILNKGAVVSGELTIAVANVDLLYLSLQLVITSVETAKREMLYVRH

SEQ ID NO: 262 - protein, 116 aa, Thiocapsa rosea
  source 1..116: note = strain DSM 235
MSVQRSTLTHSTNSTSVADLLERVLDKGIVIAGDIRIKLVDIELLTIQLRLVICSVDKAREMGIDWWSDNAMFKGLSSQASAASLPGTAAASGIEDRLARLESLLVKQSAAAETVL

SEQ ID NO: 263 - protein, 121 aa, Tolypothrix sp
  source 1..121: note = PCC 7601
MADILERVLDKGIVIAGDISVSIASTELLHIRIRLLISSVDKAKELGINWWENDPYLSSKSQRLVEENQQLQQRLESLEAQLRSLTAAKINNPELFPVNAEDNGQSDEENVPLPMNYQPND

SEQ ID NO: 264 - protein, 54 aa, Trichodesmium erythraeum
  source 1..54: note = IMS101
MFIRVDFLLDKGVIVDAWVRLSLVVIELLTIEAKIVIASVEAYLKYSEAFCFNY

SEQ ID NO: 265 - protein, 75 aa, Trichodesmium erythraeum; identical to SEQ ID NO: 58
  source 1..75: note = IMS101

SEQ ID NO: 266 - protein, 72 aa, Trichodesmium erythraeum; identical to SEQ ID NO: 59
  source 1..72: note = IMS101

SEQ ID NO: 267 - protein, 103 aa, Trichodesmium erythraeum
  source 1..103: note = IMS101
MKTSANIATSASGNGLADVLERVLDKGVVIAGDISVSIASTELLNIKIRLLISSVERAKEIGINWWESDPYFSSQNNSLVQANEKLLERVASLESEIKALRSN

SEQ ID NO: 268 - protein, 104 aa, Trichodesmium erythraeum
  source 1..104: note = MS101
MKTSANIAKSAGGDSLADVLERVLDKGIVIAGDISVSIASTELLNIKIRLLISSVERAKEIGINWWESDPSLSSQNNSLVQVNQKLLERVASLESEIEALKYSQ

SEQ ID NO: 269 - protein, 155 aa, Anabaena-flos-aquae
MVCTPAENFNNSLTIASKPKNEAGLAPLLLTVLELVRQLMEAQVIRRMEEDLLSEPDLERAADSLQKLEEQILHLCEMFEVDPADLNINLGEIGTLLPSSGSYYPGQPSSRPSVLELLDRLLNTGIVVDGEIDLGIAQIDLIHAKLRLVLTSKPI

SEQ ID NO: 270 - protein, 94 aa, Bacillus-megaterium
MQPVSQANGRIHLDPDQAEQGLAQLVMTVIELLRQIVERHAMRRVEGGTLTDEQIENLGIALMNLEEKMDELKEVFGLDAEDLNIDLGPLGSLL

SEQ ID NO: 271 - protein, 101 aa, Ancylobacter aquaticus
  source 1..101: note = strain UV5
MTAPCTAETLENALRGRIDIDPEKVEQGLVKLVLMLVETVRQVVERQAIRRVEGGTLTEEETERLGLALMRLEEKMAELRLHFGLEDGDLDLKLQLPLGEL

SEQ ID NO: 272 - protein, 169 aa, Aphanizomenon flos-aquae
  source 1..169: note = NIES-81
MVYSPVENSNDFLNVIPVENSNEFLNTSPKKKSNSETGLAPLLLTVLELIRQLMEAQIIRRMEEDLLSESDLERTAESLQKLEEQILNLCQIFDIDPADLNINLGDFGSLLPASGSYYPGETGNRPSILELLDRLLNTGIVVDGEIDIGVAQLDLIHAKLRLVLTSKPI

SEQ ID NO: 273 - protein, 157 aa, Aphanothece halophytica
  source 1..157: note = strain PCC 7418
MSADESNLSQVNLNPATSNSDAGLAPLLLTVTELIRQLMEAQVIRRMDGGLLNEEELDRAGDSLQRLEAEIIRLCEIFEIDPKDLNVDLGELGTLMPKNGGYYPGESSDDPSILELLDRILHKGVVIDGNLDLGIAQLSLIQARLHLVLTSQPINGK

SEQ ID NO: 274 - protein, 105 aa, Aquabacter spiritensis
  source 1..105: note = strain DSM 9035
MTGFAGGPAVTETLESVLQGRVDIDPERVEQGLVKLVLMVVETLRQVIERQAIRRVEAGALTDEEIERLGLTLLRLEEKMAELRVQFNLSEADLSLKLRLPLGEL

SEQ ID NO: 275 - protein, 119 aa, Bradyrhizobium oligotrophicum
  source 1..119: note = S58
MSASSHSEAPGLRLQLGDLDTALAAVFTDAAPNGSINLDPDKIEHDLARLVLTLIEFLRRLLELQAIRRMEANELSEDEEERVGLALMRAAAQVSRLARELGVDPRELNLQLGPLGRLL

SEQ ID NO: 276 - protein, 120 aa, Burkholderia thailandensis
  source 1..120: note = sp. Bp5365 strain MSMB43
MNAPHAAAVSDAAALAAALEQALAQQQAPPPRATQRFDVATASAGNGLAKLVLALMKLLHELLERQALRRIEAGSLNDDEIERLGLALMRQAEEIERLAAQFGFTDADLNLDLGPLGRLF

SEQ ID NO: 277 - protein, 114 aa, Chlorobium luteolum
  source 1..114: note = DSM 273
MHEDKVQFQASSVEEALRQLEGMKQGKESRIEANPDNVESGLARLVLTLIELLRKLMEKQAMRRIDGGSLDEAQIDELGETLMKLEMKMDELKKTFNLTDSDLNLNLGPLGDLM

SEQ ID NO: 278 - protein, 156 aa, Dactylococcopsis salina
  source 1..156: note = PCC 8305
MSEEESNLSRVDLNPASSNSDAGLAPLLLTVTELIRQLMEAQVIRRMDAELLTEAELDRAGESLQRLEEEILRLCEIFDVDPADLNVHLGELGTLLPKEGGYYPGETSDQPSILELLDRVLHTGVVIDGNLDLGIAQLNLIQAKLHLVLTSQPINN

SEQ ID NO: 279 - protein, 128 aa, Desulfobacterium vacuolatum
  source 1..128: note = DSM 3385
MIKDPEAKDFKIESDSIDAFARVMHADTSSCSSSSVTAGQRQQRLKIDEENIKNGLAQLVMTLIKLLHELLERQAIRRIESGSLDDDQIERLGLTLMQQCEEIDRLRKLFDLEEEDLNLDLGPLGKLL

SEQ ID NO: 280 - protein, 121 aa, Desulfomonile tiedjei
  source 1..121: note = DSM 6799
MNPMNIAKVESDSLGDFAEIMQTDWISSLHSDKEEKRLNLNQDSVKNGLGQLVLTLVKLLHDLLERQAIRRMEAGTLTDTEIDRLGTTLMMQAQEIERLRSEFGLEEEDLNLDLGPLGKLL

SEQ ID NO: 281 - protein, 104 aa, Desulfotomaculum acetoxidans
  source 1..104: note = DSM 771
MYIDISEGSLKQGVLGLLLALVEIIKDALKIQALKRIEGDSLTEDEIERLGNALHELEEALVEIEMEHNLQNVVQNIREGLDNVVNEVVDTFNPERWIAENEFN

SEQ ID NO: 282 - protein, 155 aa, Dolichospermum circinale
MLSTPADNFDESLTTVSKSKNEAGLAPLLLTVLELLRQLMEAQVIRRMEDNLLSESELERAADSIQKLEEQILHLCETFEVDPAELNINLGDFGTLLPQSGSYYPGETGSRPSVLELLDRLLNTGVVLDGEIDLGLAQLDLIHAKLRLVLTSKPI

SEQ ID NO: 283 - protein, 94 aa, Enhydrobacter aerosaccus
  source 1..94: note = strain ATCC 27094
MTKLLEAKTVDPDKAGDDLVKLVLALVETLRQLVERQAIRRVDSGVLNDDEVERLGLALLRLEEKMSELKAHFGFGDEELTLKLGSLGELARDV

SEQ ID NO: 284 - protein, 146 aa, Isosphaera pallida
  source 1..146: note = ATCC-43644
MSDSLFEVRSPSAAPPSPVNPGVADEWTAVLKDWDTLTAQLRQATAPPNAENSARSHATTGRIDLDPEQVGDGLAKLVLTLLELIRQLLERQAIRRLDAGSLDHEQTERLGLTLMRLAQRMEELKTHFGLQGEDLNLDLGPLGKLL

SEQ ID NO: 285 - protein, 98 aa, Legionella drancourtii
  source 1..98: note = LLAP12
MNDKREEDNALPQRINLQPDDVKNGLGKLVLILIQLIHELLERQAIGRIEAGDLSDEQIDRLGITLMKQAEEIDKLREVFGLTQEDLNLDLGPLGKLL

SEQ ID NO: 286 - protein, 157 aa, Microcystis aeruginosa
  source 1..157: note = NIES-843
MTLACTPYDSDNQALLTRPESNSQAGLAPLLLTVVELVRQLLEAQIIRRMEKGVLSESDLDRAAESIQKLQEQILYLCEIFEVEPEELNVHLGEFGTLLPEAGSYYPGEEGIKPSVLELVDRLLNTGVVVEGNVDLGLAQLDLIHLKLRLVLTSQPV

SEQ ID NO: 287 - protein, 144 aa, Nostoc punctiforme
  source 1..144: note = ATCC 29133
MQAISKSKGSDSGLAPLLLTVVELIRQLMEAQVIRRMDAGTLNDSELDRAAESLQKLEQQVVQLCEIFDIDPADLNINLGEMGNLLPQSGGYYPGETSSQPSILELLDRLLNTGVVVEGDLDLGLAQLSLVHAKLRLVLTSKPL

SEQ ID NO: 288 - protein, 155 aa, Nostoc sp
  source 1..155: note = PCC 7120
MVCTPVEKSPNLLPTTSKANSKAGLAPLLLTVVELIRQLMEAQVIRRMEQDCLSESELEQASESLQKLEEQVLNLCHIFEIEPADLNINLGDVGTLLPSPGSYYPGEIGNKPSVLELLDRLLNTGIVVDGEIDLGLAQLNLIHAKLRLVLTSRPL

SEQ ID NO: 289 - protein, 132 aa, Octadecabacter antarcticus
  source 1..132: note = 307
MKTTSDSQFDSMKKILTDSSKEDSASCDPTDLLPNKSLPPSLSTSPETAADDLVKLVLAVIDTVRQVMEKQAIRRVESGALAEAEIERLGLTLMRLEARMVELKSHFGLSNEDLNLHFGTVQDLKDILNDEE

SEQ ID NO: 290 - protein, 132 aa, Octadecabacter arcticus
  source 1..132: note = 238
MKTQNDTQFDSMKKILTDSGGGDPNPNGSPDQTQHASLPSNLSTDPETAADDLVKLVLAVIDTVRQVMERQAIRRVDSGALADEEIERLGLTLMRLEERMADLKSHFGLSNEDLNLNFGTVQDLKDILNDEE

SEQ ID NO: 291 - protein, 114 aa, Pelodictyon phaeoclathratiforme
MDSDKILYYAGSADEIIEELEKLKPGIQGRINATPDNVESGLAKLVLTLIELIRKLIEKQAMRRIDGNSLSESQIEELGETLMKLEKKMEELKGIFNLTDKDLNLNLGPLGDLM

SEQ ID NO: 292 - protein, 156 aa, Phormidium tenue
  source 1..156: note = NIES-30
MTSENAEPDLSTTLALQPPAKTDAGLAPLLLTVIELVRQLMEAQVIRRMESGDLDDNDLERAADSLRKLEEQVVSMCEIFDVDPADLNIDLGEIGTLLPKEGNYYPGQKNQNPTILELLDRLLDTGVVVEGDVDLGMAQLNLIHAKLRLVLTSKPI

SEQ ID NO: 293 - protein, 153 aa, Planktothrix agardhii
  source 1..153: note = str. 7805
MSSSEPSIETIITPKSSRKDAGLAPLVLTLVELIRQLMEAQVIRRMEGNTLSEEELDRAAQSLQQLEIQVLKLCEIFEIDPTDLNIELSEFGTLLPKSGSYYPGENTQNPSILELLDRLMNTGIVVEGSVDLGLAQLNLIHAKLRLVLTSKPL

SEQ ID NO: 294 - protein, 110 aa, Psychromonas ingrahamii
  source 1..110: note = 37
MPFEHFKSNNQADVNSDTKPAASVGGLNLESDDLKNGLGRLVLTLVKLLHELLERQALRRMDAGSLQDDEIERLGLAFMKQAEEIDRLRKEFGLEVEDLNLDLGPLGRLL

SEQ ID NO: 295 - protein, 112 aa, Rhodobacter capsulatus
  source 1..112: note = SB 1003
MSAAMHLELGDVDAVLSQAARSLAAGGRLTLDPERVEQDLARLVLGIVELLRKLMELQAIRRMEAGSLTPEQEETLGLTLMRAEAALHEVAAKFGLQPADLILDLGPLGRSV

SEQ ID NO: 296 - protein, 110 aa, Rhodobacter sphaeroides
  source 1..110: note = 2.4.1
MTYPFPPLLLRDDRLPPTEAPVTAPRIALDPDRLEHDLARILLGLMEMLRQIMELQAIRRMEAGSLSESQQEQLGTTLMRAEAAIHEMAARFGLTPADLSLDLGPLGRTI

SEQ ID NO: 297 - protein, 91 aa, Rhodococcus hoagie
  source 1..91: note = 103S
MRRRIDSDPESVERGLVALVLTLVELLRQLMERQALRRVDAGDLSDDQIERIGTTLMLLEEKMEELREHFGLEPEDLNIDLGPLGPLLAED

SEQ ID NO: 298 - protein, 115 aa, Serratia sp.
  source 1..115: note = ATCC 39006
MTTNQLSHHSPVFGPTSPAIQRPITEANRHKIDIDGERVRDGLAQLVLTLVKLLHELLERQAIRRMDSGSLSDEEVERLGLALMRQAEELTHLCDVFGFKDDDLNLDLGPLGRLL

SEQ ID NO: 299 - protein, 105 aa, Stella vacuolate
  source 1..105: note = ATCC-43931
MTGFLNGPADVETLETALRGRVDIDPERVEQGLVKLVLMVVETLRQVIERQAIRRVESGSLTDDEVERLGLTLMRLEEKMDQLRRQFDLGEEDLSMRLRLPLQEL

SEQ ID NO: 300 - protein, 127 aa, Thiocapsa rosea
  source 1..127: note = strain DSM 235
MSDTRTGTAPSSAASAAPDTSTLQRANLLADLLETKVAAAGRRIDIDPERVQRGLGQLVLTVVKLLHVLLERQAIRRVDGGDLDEDEIEQLGLALMRQSEEIERLRRLLGLEEQDLNLDLGPLGKLF

SEQ ID NO: 301 - protein, 160 aa, Tolypothrix sp.
  source 1..160: note = PCC 7601
MAMVCTPSENSNDLLATNSKANNQAGLVPLLLTVVELIRQLMEAQVIRRMEEECLSESDLERAAESLQKLEEQVLNLCQIFEIDPADLNIHLGELGSLLPAAGSYYPGETGNTPSVLELLDRLLNTGVVVDGELDLGVAQLNLIHAKLRLVLTSKPLNTK

SEQ ID NO: 302 - protein, 153 aa, Trichodesmium erythraeum
  source 1..153: note = IMS101
MSLENSPEESLIVPIDKSKSNPEAGLAPLLLTVIELLRELMQAQVIRRMDAGILSDEQLERAAEGLRQLEEQVIKLCKVFDIPTEDLNLDLGEIGTLLPKSGEYYPGEKSENPSVLELLDRILNTGVVLDGTVDLGLAELDLIHARLRLVLTA

SEQ ID NO: 303 - protein, 231 aa, Ancylobacter aquaticus
  source 1..231: note = strain UV5
MLYLYAILESPPPQKPLPPGIGGAAPLFVESHALVCAASEAADAAIAREPSQIWRHQEVVAALMEGRPVLPLRFGTVVEDSAACLRLLARHHAELSAQLDRVRHCVEFALRVAGLSELADPGLDPNATPAGLGPGASHLRTLVRRERGWPVSSAAFPHDTLTAHAASRLLWARSPSQPDLRASFLVQRRSASAFLDDVNALQRLRPDLGITVTGPWPPYSFSDPDLSGGRE

SEQ ID NO: 304 - protein, 223 aa, Aphanothece halophytica
  source 1..223: note = strain PCC 7418
MLYTYCFLFSPEKTLSLPQGFKGDLQMIEKGAIAAVVEPNLPKAELEEDDQKLVQAVVHHDWVICELFRGLTVLPLRFGTYFRGEADLRSHLAAYEESYQQKLTALTGKVEVTLKLTPIPFSEEGSSSTAKGKAYLQAKKQRYQQQSNYQTQQQEALEKLQEEIKKTYPQLIHDEPKENTERFYLLIDSHSFSVFGEKMEQWKQFLSSWSIEISDPLPPYHFL

SEQ ID NO: 305 - protein, 226 aa, Aquabacter spiritensis
  source 1..226: note = strain DSM 9035
MLYLYAVLEAPPPARSLPPGIGGGAPHFIEAFELVCAASETPNRSVAPEPAEVWRHQQVVEALIDRAPALPLRFGTLVEDASACRRLLTRHRDALGAQLGRVRHCVEFALRVSGLPEEVAPDPGIGGGPGTSYLRTLARREAGWPPSTAVFPHDGLAAHAAERLLWARSTSQPDLRASFLVRKPNVAAFLADVSALQRVRPDLGITCTGPWPPYSFSDPDLSGVSP

SEQ ID NO: 306 - protein, 269 aa, Bacillus-megaterium
MGELLYLYGLIPTKEAAAIEPFPSYKGFDGEHSLYPIAFDQVTAVVSKLDADTYSEKVIQEKMEQDMSWLQEKAFHHHETVAALYEEFTIIPLKFCTIYKGEESLQAAIEINKEKIENSLTLLQGNEEWNVKIYCDDTELKKGISETNESVKAKKQEISHLSPGRQFFEKKKIDQLIEKELELHKNKVCEEIHDKLKELSLYDSVKKNWSKDVTGAAEQMAWNSVFLLPSLQITKFVNEIEELQQRLENKGWKFEVTGPWPPYHFSSFA

SEQ ID NO: 307 - protein, 266 aa, Burkholderia thailandensis
  source 1..266: note = sp. Bp5365 strain MSMB43
MNDALYLFCFARAEPLAPAWAKRAPGEPRLQLLHEGNLAAVLCDVSRSEFAGADAERRLADPAWIAGRVAVHAAAIEWTMRYSPVIPAQFGTLFSGAGRVIALMESCHAHIGRVLDHVEGKTEWAVKGWLDRQAAADSQAALLRADEPESAARTAGARYLRERQLQARAGQNLRDWLEQSVPPISARLQRHAVEMCSRPCRASDSEHEIVANWAFLVRNRDVPAFRRQAEAIDAEFATWGLHFDFSGPWPPYSFCAPLTEETTWSG

SEQ ID NO: 308 - protein, 292 aa, Chlorobium luteolum
  source 1..292: note = DSM 273
MPCRLTVTWKSLRTAGLLPTAKGIQGRTERMAQNILYVYCIVRQLPGADIVARYPDLVFIEAGSAYVAAKYVSPLEYSDASMKLKLADEEWLDRNAREHLSVNVMIMAQQTIIPFNFGTIFKSRESLSGFLGDYGRKLDESFDALEGREEWAVKAYCNESFLLKNLHLESPAIAAIEQEIQAASPGKAYLLKKKKEAMSASALEGVHQGHAKAVWGELAALSKEHVLNRLIPEDVSGVDGRMIVNGVFLIANTDVGAFIRTTEDLGERYRDAGVFLDVTGPWPPYDFVDIPY

SEQ ID NO: 309 - protein, 225 aa, Dactylococcopsis salina
  source 1..225: note = PCC 8305
MLYTYCLIASSPSALSLPSGFRGELQLIKQGAIAAIVEAELPLEELEENDQKLIQAVIHHDAVICEIFQQIPLLPLRFGTYFPTEKDLLEHLDFKAEKYQKKLQEIQDKVELTLKLTPLPFSTENASPMEKQGKNYLKAKKQRYQEQTNYQSQQQAELNQLQTQINQDYPQFIHGEPKENIERFYLLIKERDRSVFSEQLEQWKKDFPTWTIEVSDPLPPYHFIE

SEQ ID NO: 310 - protein, 269 aa, Desulfobacterium vacuolatum
  source 1..269: note = DSM 3385
MEKKKAVYLYCVTRANKFNAPGITGIDANTPVCFEHLENFVAVYNIIPLNTFVGTSAEENMKNIDWIGPRAMRHENVIERMMQESSVYPARFATLFSSMENLRETLHLKSGLISRFLNQTQHKCEYSLKGFINRKQLLEFLIKTKFKQEKKQLDGLSPGKKYFAQHQFNKKVETGINQWIKRRCGIFLDHLTKRNPEVSPRELFTEKTEKNNLEMMFNLAFLIHNDSKSAFLQEISQAEKEFSQTGISLVVSGPWAPYSFCKTTRGEGL

SEQ ID NO: 311 - protein, 268 aa, Desulfomonile tiedjei
  source 1..268: note = DSM 6799
MSNVLYLFCLARTGLVDHIEGTGITGTEDLILKNFSGVTAVTCEVPEDDFSGESAEIKLQDLAWVGPRAVRHDRIIEEIMQYSPVFPAPFGSLFSSEKRLGTLIESNIDAIREFLDHTADKQEWSVKGLVCKSKAVDEIFTGKLKILSETLSSSPAGMRYFKERQMRSEAEKELSGKVKAACTVVGEKLLACSNNFRQRKNISFGKAEGDKQLVVNWAFLVDHSRISYFLDQVEHANSNYQAGGLAFECSGPWPPYSFCPSLHMEPTR

SEQ ID NO: 312 - protein, 259 aa, Desulfotomaculum acetoxidans
  source 1..259: note = DSM 771
MNLIDDCKAKYIYCIGENPGNWPSEVMGVEGSLVYHVVYRDIAAVVHDCAEQPYNSDDNNKVIDWVLGHQLVVDKACSCYSSVLPFTFNSIVKGKEDLSSHEILVNWLEDNYDNFKLKLGKIKGKKEYSVQLFLDKQVSLSLLQSESDILELQVELLGSAKGKAYFVQEKINKKIGELMANRADSYCRQFYHEISSVVSECKLCKLKQAGRNEIMIINLVCLAGDNEVEVLGDVLEKIKSNDIAIKIKFSGPWPAYSFV

SEQ ID NO: 313 - protein, 240 aa, Enhydrobacter aerosaccus
  source 1..240: note = strain ATCC 27094
MLYVYGIADNAFEVLRGAGLLNSDVFAVPAGCLAAAASKLAQGGIETTPQGVWRHEQVLRQLMQDHAVLPLRFGTICRDRETLTDRLMEASDDLVRGLGRVRGKVEIALRIVDEREHEAHPVPSETPTVDAIGGGRGTAYLRARRRHHAAEMGREARAERVGKMLSAYIDVGAEDLVCSVAPEGDHAVSVSCLLGRDQLATLQAALERFQSDHPAIGLSWTGPWTPYSFVAPSLFGVGLP

SEQ ID NO: 314 - protein, 266 aa, Legionella drancourtii
  source 1..266: note = LLAP12
MNKALYLFCLTPASDLPMMEGELLPNFSPLFIHPFQTFNAILSWVPAKEYQEQSTDSNLINTEEFMQRVFFHELVVEKIMRDEAVFPIGFGTLFSSIASLEEQILTHQTLISSCLANLNQKDEYAVRVYLNQDKALESLLSVMLQERESSWASSSPGVQYLKKQQLHNEIQRNLNQHLGGMLDEVLSMFQRHATDFKSRENTAQSSDIHGTSILHWAFLIPRVVSSIFKEQVDLMNAKYNPFGLHFVLTGPWPAYSFCTLQSVEAP

SEQ ID NO: 315 - protein, 240 aa, Lyngbya confervoides
  source 1..240: note = BDU141951
MRWHRSEAVISYCDLSMIYLYALCPNSTETNNLPEGIGTAQVEVLTVGTLGAVIERDVDIAQIQKDDAQLMAAVLAHDRILSHLFTYSPLLPLRFGTQFSNSEAVTTFLKTQGETYRQKLSHLQDRAEYLVKLIPQPLDLPAIASDLKGREYFLAKKQRLQDHTAALNQQADELQTFLTDLATQDIPLVRSAPQDHEERLHVLLSRDTDTTEQVIMTWQEQLPNWQVVCSEPLPPYHFAA

SEQ ID NO: 316 - protein, 240 aa, Octadecabacter antarcticus
  source 1..240: note = 307
MKRLYVYGIVGATSFDDPLPNGHDEASVFALVSGDIAVAVSFVERSAVEASAANVWLHDNVLSALMTRYAVLPMRFGTIAVGATQLLEGIVKRQKQLMKDLMRLNENVEIALHISGKNWEKVNQKVTKKNTDQAITQGTAYLLGRQQSLYGSDKTQLLVQNVRRAIRSGLDPLMKDVIWPIDKPQALPFKASCLINRNDVASFVQIVNDIAAQNLDARVTCTGPWAPYSFVGKSGVEGET

SEQ ID NO: 317 - protein, 240 aa, Octadecabacter arcticus
  source 1..240: note = 238
MTKLYVYGIVGATHFDVKLPNGHDEAPVFAIVSGDLAVAVSSLERSAVEASAANVWLHENVLSALMEGHAVLPMRFGTIATGAAQLLGDIVKRRGQLMKDLTRLDGKVEIALRISGKNREKVEQRIAGQIVDTNVTQGVAYLQEKQQNLYGSFYTQSSVQCARRAIRSQLDPFIVEAIWPTDEPQMLPFRASCLIKKGDIARFVQTVDDVVVKVSDIRVTCTGPWAPYSFVGQSGSEAET

SEQ ID NO: 318 - protein, 266 aa, Pelodictyon phaeoclathratiforme
MVAIQERLIYIFCVTSEPPLLQQYQLQKGICVVDVDGLFVTTMDVTDNDFAENQLQSNLSDVVWLDTKVREHLDVITSIMQHVKSLIPFNFGTLYKSESSLMQFIIKYAEEFKKNLVYLEEKEEWAVKLYCNKNKIVENITHLSKKVSDINALIQNSSIGKAYILGKKKNEIIENEIINIYNTYSKKIFTKFSILSEEFRFNPIPNNETLEKEDDMILNVVLLLNKANVESFIETSDQLIIQHQNIGLNIEITGPWPCYSFINISH

SEQ ID NO: 319 - protein, 249 aa, Pelodictyon phaeoclathratiforme
MPLIIYAIFDSINYIDSFSSYVDAISLKSKIKLEIISTSTLSAIVSRTTDEKKQACQNDVMIYATIIGDIAAKYSILPMRYGSIVSSPFDVTELLKNHNETFVTIIKKITDKEEYSLRILYSHQDKEKNNIEDLFDLPQNVPDILHGNTDSKKYLLNKYIKHLSEEKRLQYIDKIQSIVACNLQKITDLIVYNKQTTTGFIVDAVFMIERSKKSELLDLVIQMQTLFSEHNVVLSGPWPPYNFSNINIG

SEQ ID NO: 320 - protein, 270 aa, Psychromonas ingrahamii
  source 1..270: note = 37
MKNSNHSGLDPNQALYLYCFVHADSIQSVTSQAIEKDSPVFIYQWQDIAAVLSHVPTSYFTGYDDEEPEQTIARILPRTQLHEQVIEEVMRQSPVFPAQFGTLFSSQESLEQEISQQYLAITHTLKEVSGSVEWAVKGVLDRGVAEKALYSQQLTEQQNSLSSSPGMRHLQEQRLRRETQSKLNSWLHQLYTDIATPLSELSGDFFQRKIPSSIEEGKEVILNWAFLVPESAGDDFHAQIDKLNQRLNSFGLVIQCSGPWPPYSFCNQSS

SEQ ID NO: 321 - protein, 270 aa, Psychromonas ingrahamii; identical to SEQ ID NO: 320
  source 1..270: note = 37

SEQ ID NO: 322 - protein, 262 aa, Serratia sp
  source 1..262: note = ATCC 39006
MTMNTEAQTEQAIYLYGLTLPDLAAPPILGVDNQHPINTHQCAGLNAVISPVALSDFTGEKGEDNVQNVTWLTPRICRHAQIIDSLMAQGPVYPLPFGTLFSSQNALEQEMKSRATDVFVSLRRITGCQEWALEATLDRKQAVDVLFTEGLDSGRFCLPEAIGRRHLEEQKLRRRLTTELSDWLAHALTAMQNELHPLVRDFRSRRLLDDKILHWAYLLPVEDVAAFQQQVADIVERYEAYGFSFRVTGPWAAYSFCQPDES

SEQ ID NO: 323 - protein, 226 aa, Stella vacuolate
  source 1..226: note = ATCC-43931
MLYLYAVLEALPAARTLPAGIGGGELLFVEAFELVCAASETPERAIAPEPTQVWRHQQVVEALIDCAAALPLRFGTLVEDAVACRRLLTRHREALCAQLDRVRHCVEFALRVSGLREEVGSDHVIGGGPGVSYMRALARREASWPPSTGTFPHDGLAAHAADRLLWSRSASQPDLRASFLVLKPNVAAFLADVSALQRMRPDLGITCTGPWPPYSFSDPDLSGMSP

SEQ ID NO: 324 - protein, 250 aa, Thiocapsa rosea
  source 1..250: note = strain DSM 235
MDAFYCFCFAPACLASDLRFDDCGWEDPIEIRRLAGLDVILSRVPLGRFAGAEAEQRLADLEWLVPRAQAHDRVITRTMERSTVFPLTFATLFSSLPALALEVAARRRALLDFFERMAGREEWAVKVSMDRERVIATRMQSLYPEGGDVPAGGRGYLLKQRRRGEAEQAIGPWLKGQIGCLDEALRPSCETLLIRPLRDEMVASRACLVARDLGPSLSEAIERSREAFADQGLDLHCSGPWPLYSFCGTP

SEQ ID NO: 325 - protein, 225 aa, Trichodesmium erythraeum
  source 1..225: note = IMS101
MSYYVYGFLYLPESCLALPKGMEKEVELVPYQNIAAVVEANVSIEAIQETEEKLLEAILAHDRVVREIFQQVSMLPLRFGNAFALRENIINDLQNNQQQYLNILTKLQQQAEYTITFTPVSYPSTLEVSKVRGKAYLLAKKQQFEQQQAFQTKQRQQWENIRQLIFKNYPKAVFRDSTESKIKQVHLLANRDARVITTEELSTWQTECSYWQITLSEQLPPYHFV

SEQ ID NO: 326 - protein, 335 aa, Anabaena flos-aquae
MTTTKVNHKRAVLRLRPGQFVVTPAIERVAIRALRYLKSGFPVHLRGPAGTGKTTLAMHLANCLDRPVMLLFGDDQFKSSDLIGSESGYTHKKVLDNYIHSVVKLEDEFKQNWVDSRLTLACREGFTLVYDEFNRSRPEVNNVLLSALEEKILSLPPSSNQPEYLSVNPQFRVIFTSNPEEYAGVHSTQDALMDRLVTISMPEPDEITQTEILIQKTNIDRESANFIVRLVKSFRLATGAEKTSGLRSCLMIAKVCADNNIPVTTESLDFPDIAIDILFNRSHLSMSESTNIFLELLDKFSAEELEILNNRVTGDNDFLIDNSQFVSQQLAGQPN

SEQ ID NO: 327 - protein, 357 aa, Ancylobacter aquaticus
  source 1..357: note = strain UV5
MTSEAASKDPISLLSGFGAGAASSGPKAGGRSTPSALTPRPRTGFVEAEQVRDLTRRGLGFLNAGYPLHFRGPAGTGKTTLALHVAAQLGRPVIIITGDNELGTADLVGSQRGYHYRKVVDQFIHNVTKLEETANQHWTDHRLTTACREGFTLVYDEFTRSRPETHNVLLGVFEERMLFLPAQAREECYIKVHPEFRAIFTSNPQEYAGVHASQDALADRLATIDVDYPDRAMELAVASARTGMPEASAARIIDLVRAFRASGDYQQTPTMRAGLMIARVAAQEGFEVSVDDPRFVQLCSDALESRIFSGQRAEEVAREQRRAALHALIDTHCPSAAKPRARRAGGAVRASIEGAQS

SEQ ID NO: 328 - protein, 359 aa, Aphanizomenon flos-aquae
  source 1..359: note = NIES-81
MTKTNHKRAVLRVRPGQFVVTPAIEQVAIRALLYLKSGFPIHLRGPAGTGKTTLALHLAHCLDRPVMLLFGDDEFKSSDLIGSESGYTHKKLLDNYIHSVVKVEDEFKQNWVDSRLTLACREGFTLVYDEFNRSRPEVNNVLLSALEEKILSLPPSSNQPEYLSVSPQFRAIFTSNPEEYCGVHSTQDALMDRLVTINMPEPDEITQTEILIQKTNIQKESAHLIVRLVKSFRIATGAEKTSGLRSCLMIAKVCADNNLVAEPENSFFQEIAMEILSNRTHLSVNESTDIFLDVISQFSNKEIEILNDAELGSLPTMDTLANTDLGNDVPLEKEASDYVIQQKNNEFKGFQKPSTKVLN

SEQ ID NO: 329 - protein, 337 aa, Aphanothece halophytica
  source 1..337: note = strain PCC 7418
MTTVLHARPKGFVSTPTIDRISRRAWRYLQSGFSIHLRGPAGTGKTTLAMHLADLLNRPIMLLYGDDEFKSTDLIGSNTGYTRKKVVDNYIHSVVKEEDELRQQWVDSRLTMACREGFTLVYDEFNRSPPEVNNVLLSALEEKLLVLPPDSHRSEYVRVSPNFRAIFTSNPEEYWGVHGTQDALLDRVVTINVPEPDLETQREIIVQKVGINADDGDMIVNFVRNFRDRAEMENSSGLRSCLMIAQVCHQHEIPVQTSNEDFQDICYDILTSRCPLSTQESISLLEQLFREYELELVVEDEDEDVPSVIVEGETEDLSSDEKPHLRLSHPFGNTEND

SEQ ID NO: 330 - protein, 364 aa, Aquabacter spiritensis
  source 1..364: note = strain DSM 9035
MSTEPAPLVSPSQDVETTPQRPARPEPAEALAVGYRLSARPASPATLTPRPRADFVETDQVKDLTRRGLGFLRAGYPLHFRGPAGTGKTTLALHVAAQLGRPVIVITGDNELGTADLVGSQRGYHYRKVVDQFIHNVTKLEETANQRWTDHRLTTACREGYTLVYDEFTRSRPETHNVLLGVFEEKILFLPAQAREECYIRVHPDFRAIFTSNPQEYAGVHASQDALADRLATIDVDYPDRGMELAVASARTGLGETEAARIIDLVRAFRASGDYQQTPTMRASLMIARVAAQEGLRVSIDDPGFVQLCMDALESRMFSGARLEAATRETSRAALLALLAVHCPSEAPIVRVTAARRAKKADAS

SEQ ID NO: 331 - protein, 394 aa, Arthrospira platensis
  source 1..394: note = NIES-39
MTTVLRAVPKGFVNTPAIERITVRALRYLQSGFSVHLRGPAGTGKTTLALHLADLLNRPIMLIFGDDELKSSDMIGNQTGYTRKKVVDNFIHSVVKLEDSLKQNWIDSRLTLACREGFTLVYDEFNRSRPEVNNVLLSALEEKLLVLPPNNSRSEYIRVNPHFRAIFTSNPEEYCGVYSTQDALLDRLITMNMPEPDEATQQEILIQKVAVTPEEAQTIVTLVQQFREATHAIAPSKIQTVARQQTNADKASGLRPSLMLARICQEHNIPIVPIDPDFQEVCRDILLSRAIGDITELESRLHQIFDHLSGLENDQIIALPPREELTTSSVPNNLSDTEQKIYTYIKDSDGARVSEIEIALGLNRVQTTDALRSLLRKSYLTQQDNRLFVVYEGD

SEQ ID NO: 332 - protein, 308 aa, Bacillus-megaterium; identical to SEQ ID NO: 11

SEQ ID NO: 333 - protein, 351 aa, Bradyrhizobium oligotrophicum
  source 1..351: note = S58
MLRSDRAAIAGGQRGSRAQGDAVARNDAAAGSRAAIAQISPRPDADNAALSPAPRTDLFENPQLASMAARALTYLNAGIPVHLRGPAGTGKTTMAMQLAARLGRPVVLLTGDDGLTAAHLVGREIGTKSRQVVDRYVHSVRRVETETSSMWCDAVLAQAVVEGLTFVYDEFTRSPPQANNPLLSVVEERILIFPAGSRKERLVHAHPEFRAILTSNPEEYAGVSRPQDALLDRLITFDLDDYDRETEIGIVSNRTGLAYAEAGVIVDLVRGVRRWPKAHHPPSMRSAIMIARIVARELITPSVDDPRFVRLCLDVLAAKAKPTDRDDRDRFAATLLRLMNNHCPAGAIDGG

SEQ ID NO: 334 - protein, 287 aa, Burkholderia thailandensis
  source 1..287: note = sp. Bp5365 strain MSMB43
MEASAEFVQTPAVRNLTERALTYLGAGYGVHLAGPSGTGKTTLAFHIAAQLGRQVVLMHGDDELGSADLVGRGAGYRRSRVVDNFIHSVVKTEEEMTTTWIDNRLTTACQHGLTLIYDEFNRSRPEANNALLPVLSEGILNLPNRMTGAGYLTVHPGFRAIFTSNPEEYVGVHKTQNALMGRLITIQVGHYDRETEVEIVRARSGIARADAERIVDLTRRLRDADDNGHHPSIRAAIALARALSYCGGEATPDNAGYVWACRDILGVDLEQDARTRSQAGRRTKARR

SEQ ID NO: 335 - protein, 308 aa, Chlorobium luteolum
  source 1..308: note = DSM 273
MRAAVNDNEMNTVLAPRPMANFVETEYIRDITERGLTYLKAGFPVHFRGPSGTGKTTVAMHLAGKIGRPVVVIHGDSEYKTSDLIGSEQGYKFRRLNDNFIHSVHKYEEDMSKQWVNNRLSIAIKKGFTLVYDEFTRSRPEANNILLPILQEKMLSTSASNEEDYYMKVHPEFRAIFTSNPEEYAGVNRTQDALRDRMVTMDLDYFDYETELRVTHAKSELTLEDSEKIVQVVRGLRESGKTEFDPTVRGSIMIARTLHIMQVRPEKTNDAVRKVFQDILTSETSRVGSKTNQEKVRAIVNDLIEAYL

SEQ ID NO: 336 - protein, 273 aa, Dactylococcopsis salina
  source 1..273: note = PCC 8305
MTTVLHARPKGFVSTPTIDRISGRAWRYLQSGFSIHLRGPAGTGKTTLAMHLADLLNRPIMLLYGDDEFKSTDLIGSNTGYTRKKVVDNYIHSVVKEEDELRQQWVDSRLTMACREGFTLVYDEFNRSPPEVNNVLLSALEEKLLVLPPDSNRSEYVRVSPNFRAIFTSNPEEYWGVHGTQDALLDRVVTINVPEPDLETQQEIITQKVGINANDGEKIVNFVRQFRDRAAVKNSSGLRSCLMIAQVCHQHEIPVQTSDEGFRDICYDILSSR

SEQ ID NO: 337 - protein, 316 aa, Desulfobacterium vacuolatum
  source 1..316: note = DSM 3385
MSASMSSMKETRQRMSAPEQDNVVPEAGSDFVETPYVKDITDRALAYLHVGYPVHFSGPAGTGKTTLAFHVAAKLKRTVMLIHGDDEFGSSDLIGKDSGYRKAKVVDNYIHSVVKTEESMNTVWADNRLTIACQQGCTLVYDEFTRSRPEANNAFLSVLEEKILNIPSLRDIDQGYLQVHPEFRAIFTSNPEEYAGVHKTQDAMMDRLITITLDHFDRDTEVQVTMSKSDLPQKDAEKIVDIVRKLRKTGVNNHRPTIRACIAIGKILKHMGGGASKDNFVFKQICRDVLNVDTTKVTRDGEPLLPRKIDELINSL

SEQ ID NO: 338 - protein, 337 aa, Desulfomonile tiedjei
  source 1..337: note = DSM 6799
MNGAELRIASIETEVITANNENIVPEAGDRFVNTPHVEELTARAMAYLEVGYSVHFSGVAGTGKTTLAFHAAAKLGRPVILVHGDHEFGSSDLIGRDAGYKKSRLVDNFIHSVVKTEEEMRSLWVDNRLTTACRDGYTLIYDEFTRSRPEANNVLLSILEEKILNLPSLRRTGEGYLEVHPSFRAIFTSNPEEYAGVHKTQDALMDRIITINVDHYDRETEIEITRAKSGVCKQDATVIVDIIRELRLLGVNNHRPTIRAAIAIARVLAHTGEHADQHNSVFQWLCKDVLSTDTVKVSRGGSPLMAKKVEEVIRKVCGRTGGKRSGKPVGSKEETSE

SEQ ID NO: 339 - protein, 307 aa, Desulfotomaculum acetoxidans
  source 1..307: note = DSM 771
MQLNGLDKNSIINPVVLSDFVVTDYISNVVDRALAYIKAGFAIHLRGRSGTGKTSIAMYISSKLNRPTLVIHGDEEFRTSDLIGGRYGYRIRKTIDNFVQSVVKVEEDLVERWVDSRLTTACKNGYTLVYDEFTRSRPEANNILLSVLQERLLDISVARGAEEGYVKVHPDFTAIFTSNPEDYAGVYGSQDALRDRMVTLDLDNYDKETEISIIKSKSKLSREDSERVVNILRDLRELGDCEYGPTIRGGIMIAKTLQVLGAPVDKNNEMFRQICEEVLASETSRAGNLQALRKVRKVINELFNKYA

SEQ ID NO: 340 - protein, 352 aa, Dolichospermum circinale
MSITKVNHKRAVLRLRPGQFVVTPAIERVVIRALRYLRSGFPIHLRGPAGTGKTTLGMHLANCLDRPVMLLFGDDQFKSSDLIGSESGYTHKKLLDNYIHSVVKVEDEFKQNWVDSRLTLACREGFTLVYDEFNRSRPEVNNVLLSALEEKILSLPPSSNQPEYLSVNPQFRVIFTSNPEEYCGVHSTQDALMDRLVTINMPEPDEITQTEILIQKTNIGRESANLIVRLVKSFRLATGAEKTSGLRSCLMIAKICADHDIPASTEDLDFREIAIDILFNRAQLSISESTDIFMGLLEQFSAEEIKVLNDTHFPTDELLINNSQFITQELVTQPNTELATDIPQELRKTEQN

SEQ ID NO: 341 - protein, 321 aa, Enhydrobacter aerosaccus
  source 1..321: note = strain ATCC 27094
MSMDQAEEIGVVTTIEPRPRADFVRTQSVEATARRALGYLNAGFSVHFRGPAGTGKTTLALHLAALLGRPMVMITGDEEMLTSTLVGTQHGYHFRRVVDRFIHTVTKTEETADKRWADHRLTTACREGYTLIYDEFTRSRPEANNVLLSVLEEGLLVLPAQNQNEPYIKVHPNFRVIFTSNPQEYAGVHDAQDALGDRIVTIDMGHADRELELAIAAARSGLPPTQVAPIVDMVREFRETGEYDQTPTLRTSIMICRMMSQERLAPTIEDQQFVQICMDILGGKSLPGGKGDNKRAQQQKMLLSLIEHHCPARSFTSVGEV

SEQ ID NO: 342 - protein, 345 aa, Isosphaera pallida
  source 1..345: note = ATCC-43644
MDYESTALQLKPRPDFVATPWVRELADRALGYLTAGYPVHFSGPAGTGKTTLAMHLAALVNRPVVLLHGDDEFGSSDLVGDHLGFRSTKVVDNFIHSVVKTEQSVSKTWVDHRLTTACRHGFTLIYDEFNRSRPEANNILLTILEERLLELPPIAGGRDGSGPLRVHPEFRAIFTSNPEEYAGVHKTQDALLDRMITISMGGHDEATETEITAAKSGLSRDEAARIVELARAVRALKPLRHPPTIRSCLMIAKVAALRKVPIDPNDALFLAICRDVLRIDALPVDDPEATFAELIRRVFAPTPAVAPPRVPTTGFAANRVVPIPRRPLAASASPPPGANGHAHLR

SEQ ID NO: 343 - protein, 335 aa, Legionella drancourtii
  source 1..335: note = LLAP12
MMTQENNGSLTDSKNNDKLIRFVNNRSDNILLEASEEFTETPHIRGISERALAYLDIGYPIHLLGPAGTGKTTVALHIAAQLGRPVILIHGDDEFTGADLVGRGTGYHHSKLVDNFIHSVLKTEEEMTTMWTDNRLTTACEQGYTLIYDEFNRSRAEANNALLSVLSEGILNLPGRRERDGIGYVDVHSNFRAIFTSNSEEYVGIHKTQNALADRLIAIKMDYPDQQSEIQIIEKKSTLPRKDIEIIVNLARELRLKSEKRPSIRGCIAIARVLAYHNRHAHADDPIFQAVCQDIFGISKEFLKQLLHPMDSGLQKRSEKNQESIKKYKTKNQKL

SEQ ID NO: 344 - protein, 421 aa, Lyngbya confervoides
  source 1..421: note = BDU141951
MSTVLQARPRNFVSTPAVERIARRALRYLQSGYSVHLRGPAGTGKTTLALHLADLLSRPIMLVFGDDEFKTSDLIGNQSGYTRKKVVDNYIHSVVKVEDELRHNWVDSRLTLACREGFTLVYDEFNRSRPEVNNVLLSALEEKLLVLPPSGHRPEYLRVNPHFRAIFTSNPEEYAGVHGTQDALLDRLITIHMPEPDELTQQQILIQKVGIEPADALMIVRLVKAFKSQMGNHSATSLRPSLMIANICHEHGVAMMTEDADFRDVCSDVLLSRVTNELSPATHTLWDLFNELTASADVLGPESNSTDVSPQPEADKPVETKGSKGKSTTKSKAKESAKASEEADEAGDDSASAPELDEIESSILTFLTARESASLSEIESELSLTRFKAVDALRSLVEAGYLQKQNGAGKPAIYGLVPEES

SEQ ID NO: 345 - protein, 346 aa, Microcystis aeruginosa
  source 1..346: note = NIES-843
MTVTETQTRRAVLSLRPGQFVVTPSIDQIATRALRYLNSGFSIHLCGPAGTGKTTLAMHLANCLARPVMLIFGDDDFTSSDLIGSQSGYTHKKLMDNYIHSVLKVEDELKHNWVDSRLTMACREGFTLVYDEFNRSRPEVNNVLLSALEEKILTLPPTSHQPDYLQVNSQFRAIFTSNPEEYCGVHATQDALMDRLVTINMPEPDQLTQTEILAQKTGIGREDALFIVNLVKTFRVKTATEKTSGLRSCLMIAKVCASHDIAANSADSDFRDICADVLLSRTNLSVDKSRAILWEILEDNPLESLSFLEEEEPSDAQVSTSEPSTGNQSLKAIQSLLRGNLPQRKD

SEQ ID NO: 346 - protein, 390 aa, Nostoc punctiforme
  source 1..390: note = ATCC 29133
MTTVLNASPQRFVNTPAVQRIAQRALRYLQSGFSIHLRGAAGVGKTTLAMHLADLLNQPIILLFGDDEFKTSDLIGNQLGYTRKKVVDNFIHSVIKVEDEVRQHWVDARLTLACKEGFTLVYDEFNRSHPEVNNVLLSVLEERLLVLPTNQHRAEYIRVHPQFRAILTSNPQEYCGVHATQDALMDRVITIDMPTPDELSQQEIVVHKTGIDSEKAEVIVRIVRTFWSRSGSGQGGGLRSCLMIAKICHEHEISVNPGDPSFQDICADILLSRTNQPLIEATRLLEEVLSEFYHRINTQSQPSEIIPNNQNQIVLEQRVPYEHEVYNYLCNSPGRRFSELAVELGIDRSQIVAALKSLREQGVLVQMQGNAESPSISQTVAFDSGHLINK

SEQ ID NO: 347 - protein, 410 aa, Nostoc sp
  source 1..410: note = PCC 7120
MTLTANNKKRAVLRVRPGQFVVTPAIEQVAIRALRYLTSGFAIHLRGPAGTGKTTLAMHLANCLDRPIMLIFGDDEFKSSDLIGSESGYTHKKLLDNYIHSVLKVEDEFKQNWVDSRLTLACREGFTLVYDEFNRSRPEVNNVLLSALEEKILTLPPSSNQPEYLHVNPQFRAIFTSNPEEYCGVHSTQDALMDRLVTINMPEPDELTQTEILAQKTALNRADALLIVRLVKAFRSRTGGEKTSGLRSCLMIAKVCAEHNILVSPQSSDFREICADVLFNRTNWSASEAATIFLELLNHLDLQQIEEFKNSIIPEDTDAIAEGGFPTIIDSHFGTLDSEVLEQPGVEDSIPFEQEIYLYLQQYKSAALALVQQEFELSRTVATNALNSLEQKGLVSKNNHVYTIEEPNQS

SEQ ID NO: 348 - protein, 342 aa, Octadecabacter antarcticus
  source 1..342: note = 307
MNSNLRATNSGGPDISKTMMPEAREDFVQTESVKSISRRALAYINAGYSVHFRGPAGTGKTTMAMHTAALLGRPVVLITGDEEMITSNLVGAESGYNYRKVTDNYIHTVSKIEESSDRSWNDHRLTTACREGYTLIYDEFTRSRAEANNVLLSVLEEGILVLPAQNRGEPFIKVHPNFRVIFTSNPQEYAGVHEAQDALSDRIVTIDIGEADRELEVSIASSRSGLEVAKTEPIVDMVRAFRDTGEYDQTPTLRACIVICRMVANEKLNTTIDDPFFVQICLDVLGSKSTFGGKEHDKRTQQRKLLLDNLKHYCPSKVSTKPSAKDDESKSTLIQVSSRGSL

SEQ ID NO: 349 - protein, 324 aa, Octadecabacter arcticus
  source 1..324: note = 238
MMPEARKDFVQTDSVKSVSRRALAYINAGYSVHFRGPAGTGKTTMAMHTAALLGRPVVMITGDEEMVTSNLVGAESGYNYRKVTDNYIHTVSKVEESSDRSWNDHRLTTACREGYTLIYDEFTRSRAEANNVLLSVLEEGILVLPAQNRGEPFIKVHPDFRVIFTSNPQEYAGVHDAQDALSDRIVTIDIGAADRELEVSIASSRSGLEVAKTAPIVDMVRAFRDTGEYDQTPTLRACIMICRMVANEKLNPTIDDSYFVQICLDVLGSKSMFGAKEQGKRTQQEKLLLDNLSHHCPSPPPSKPSAKEAEAKPRSIQATSRGPA

SEQ ID NO: 350 - protein, 308 aa, Pelodictyon phaeoclathratiforme
MRRQGCDSEMNTVLEPKPMPNFVETDYIRDITSRGLTYMKAGFPVHFRGPSGTGKTTVALHLASKIGRPVVIIHGDSEYKTSDLIGSEQGYKYRRLDDNFIHSVHKYEEDMTKQWVNNRLTIAIKKGFTLVYDEFTRSRPEANNILLPILQEKMMSTSSSNEEEYYMKVHPEFRAIFTSNPEEYAGVNRTQDALRDRMVTMDLDYFDYETELMITHAKSGMSLDDAEKIVKIVRGLRESGKTEFDPTIRGSIMIAKTLNVLNARPDKTNELFKKVCQDILTSETSRVGSKTNQERVRGIVNELIDLHS

SEQ ID NO: 351 - protein, 454 aa, Phormidium tenue
  source 1..454: note = NIES-30
MNTVLQARPRNFVSTPTLERTSIRALRYLQSGYSIHLKGPAGTGKTTLALHLADLLARPIMLLFGDDEFKTSDLIGNQSGYTRKKVVDNYIHSVVKVEDELRHNWTDSRLTLACREGFTMVYDEFNRSRPEVNNVLLSALEEKLLVLPPSNNRAEYIRVSPHFRAILTSNPEEYCGVHGTQDALQDRLITINMPEPDELAQQQILVQKVGIDSSAALQIVQLVKAFQSAVAPDMVSSLRPSLMIATICHDHDILPLAENADFRDVCSDILLARSKEPAPDATRHLWNLFNRFVVSQAALVNDLSLKPEAHPTARFHGEEEDDAPLQPLEALVESDIDDVAVEDQPVIGPQDLQGETLPEAVIPEPQGETVVETPAEAEALPEEIARVQVSPDDIETRIFDYLDATGTASLVNIEAALDLNRFQAVNAVKSMLDQGLIEKQETDGQLQGYQLSSN

SEQ ID NO: 352 - protein, 398 aa, Planktothrix agardhii
  source 1..398: note = str. 7805
MTTVLQARPKGFVNTPTIEQLTIRALRYLQSGFSLHLRGPAGTGKTTLAMHLADLLNRPIVLIFGDDELKSSDLIGNQLGYTRKKVVDNFIHSVVKLEDELRQNWIDSRLTLACKEGFTLVYDEFNRSRPEVNNVLLSALEEKLLVLPPNNSRSEYIRVNPHFRAIFTSNPEEYCGVYGTQDALLDRLITIDMPEPDDETQQEILIQKIGISPEDAKNIIEIVKIYLEITTQKKEIKPVQNGKAARPHIDKASGLRPGLIIAKICHEHDISIQENNQDFIKVCADILLSRTNLSLTEAQNKLEKVIKTVLTDGDTSNNSFLPPSETQLTENNSLEIEEQVYQYLQKTTSARVSEIEVALGLNRVQTTNVLRSLLKQGHLKQQDNRFFAVNKQGELIQP

SEQ ID NO: 353 - protein, 398 aa, Planktothrix rubescens
MTTVLQARPKGFVNTPTIEQLTIRALRYLQSGFSLHLRGPAGTGKTTLAMHLADLLNRPIVLIFGDDELKSSDLIGNQLGYTRKKVIDNFIHSVVKLEDELRQNWIDSRLTLACKEGFTLVYDEFNRSRPEVNNVLLSALEEKLLVLPPNNSRSEYIRVNPHFRAIFTSNPEEYCGVYGTQDALLDRLITIDMPEPDDETQQEILIQKIGISPEDAKNIIEIVKIYLEITTQKKEIKPVQNGKAARPHIDKASGLRPGLIIAKICHEHDISIQENNQDFIKVCADILLSRTNLSLTEAQNKLEKVIKTVLTDGDTSTNSFLPLSETQLTENNSLEIEEQVYQYLQKTTSARVSEIEVALGLNRVQTTNVLRSLLKQGHLKQQDNRFFAVNKQGELIQP

SEQ ID NO: 354 - protein, 334 aa, Psychromonas ingrahamii
  source 1..334: note = 37
MSIENLNNVSEIKIEQSDDDHIYPEASEDFVETPYIKEVTERAMLYLDAGYPVHFAGPAGTGKTTLAFHIAALRQRPVTLIHGNHEFGTSDLIGKESGYRRHRVVDNYVHSVVKEEEELQSLWSDNRLTTCCRNGDTLVYDEFNRSTPEANNVLLSILEEGILNLPSSRSDGYLEVHPQFRAIFTSNPQEYAGTHATQDALVDRMITIMLHYPDRHTEVRVAIAKSGINSDEAGSIVDIVNEFRELCGSKIVSSGPKTMPTVRASIAIARVLVQKGEHAFRDNTFFHRICRDVLCMYTQQVSFSNRSVLDKQLEDLIMKFCPATYKSSGSKIRA

SEQ ID NO: 355 - protein, 335 aa, Psychromonas ingrahamii
  source 1..335: note = 37
MSINNLNISTIKIEQPENDNIYPEASAEFVQTPYIQEVTERALLYLDAGYPVHFAGPAGTGKTTLAFHIAALRKRPVTLIHGNHEFGSSDLIGKESGYRRHRLVDNYVHSVMKEEEELKSLWVDNRLTTCCRNGDTLVYDEFNRSTPEANNVLLSILEEGILNLPSLRSMGDGYLEVHPSFRAIFTSNPQEYAGTHATQDALVDRMITIMLNYPDRDTEVRVAVAKSGISNEEAGFIVDIVNEFRELSNHKSLSSGQKSMPTVRASIAISRVLIQKGEHAFRDNVFFHRVCHDVLCMYIQKISPSNRSFLDKQLEVLIGKFCPAAKSALVPKVVK

SEQ ID NO: 356 - protein, 305 aa, Rhodobacter capsulatus
  source 1..305: note = SB 1003
MTIPRDLPWGDARTPLFEDEELRSLLDRAEIYLREGIAIHFRGPAGVGKTTLALHLAQRFARPVTFFVGNDWLGRADIFGRDLGETVSTVQDHYISSVRRAERKSRIDWQEAPLARAMRDGHVLVYDEFSRSRPEANAALLSVIEEGVLPLSDPAAGRSHIVAHPDFRVILTSNPRDYVGVQAVPDALLDRMITFSLDGMSFETEVGIVATAARTDPADARAICALIHLLRAEKPGTMEISMRSGIMIARLARAAGVAPDPADPVFVQICADVLGTRMRGSDIDDVMALLLRPDPAPAACAGGAR

SEQ ID NO: 357 - protein, 316 aa, Rhodobacter sphaeroides
  source 1..316: note = 2.4.1
MTVLSPSLPHAAGIDAALVENPWLGLRRSGRYFQNAETEALFARALGYARAGVCVHLAGPAGLGKTTLALRIAQALGRPVAFMTGNEWLGSRDFIGGEIGQTVTSVVDRYIQSVRRTEQSARIDWKESILGQAMRCGQTFIYDEFTRASPEANAALLSVLEEGVLVSTDGASRHQYIEAHPDFRVLLTSNPHEYQGVKAAPDALIDRMVTLRLEEPSAPTLAGIVALRSGLDPATARRIVDLILSVQRSGEMQAPPSMRTAILVARLAAPLRLAGRLSDAALAEIAADVLRGRGLEADAAAFEAKLAAPTPGETAR

SEQ ID NO: 358 - protein, 329 aa, Serratia sp
  source 1..329: note = ATCC 39006
MIKQNTVSQYTVDDDLVVPEASEHFVATSYVNDIIERALVYLRAGYPVHFAGPSGIGKTTLAFHLAALWGRPVTMLQGNEEFVSSDLTGKDIGYRKSSLVDNYIHSVLKTEEQMNRMWVDNRLTTACRNGDMLIYDEFNRSKAETNNVLLSVLSEGILNLPGLRGVGEGYLDVHPEFRAIFTSNPEEYAGTHKTQDALMDRMITINIGLVDRDTELQILHARSELELKEAAYIVDIIRELRGNEHETKHGLRAGIAIAHILHQQGIKPRYGDKLFHAICYDVLSMDAAKIQHAGRSIYREMVDGVIRKICPPIGSDTVKASTQKIKAVE

SEQ ID NO: 359 - protein, 364 aa, Stella vacuolate
  source 1..364: note = ATCC-43931
MSTEPAPVMPPSTDIEFGSQRPARPKPAEALAVGYRLSARPAAPSTLTLRPRADFVETDQVKDLTRRGLGFLRAGYPLHFRGPAGTGKTTLALHVAAQLGRPVIVITGDNELGTADLVGSQRGYHYRKVVDQFIHNVTKLEETANQRWTDHRLTTACREGYTLVYDEFTRSRPETHNVLLGVFEEKILFLPAEAREECYIRVHPDFRAIFTSNPQEYAGVHASQDALADRLATIDVDYPNRAMELAVASARTGLAEAEAARIIDLVRAFRASGDYQQTPTMRASLMIARVAAQEGLRISVDDPGFVQLCMDALESRIFSGARQEADARARHRVALLGLLATHCPSEAPVARVATVARAKRKSAS

SEQ ID NO: 360 - protein, 332 aa, Thiocapsa rosea
  source 1..332: note = strain DSM 235
MSAKPLQDASEVSALNNDNVQPEASDTFVCTPSVEALAERASAYLQAGYPVHLAGPAGTGKTTLAFHAAAKRGRPVKLIHGNDELGLADMVGQDNGYRRNTLVDNYIHSVVKTQEEVRTFWIDNRVTTACLNGETLIYDEFNRSRPEVNNIFLSILGEGILNLPNRRHQGAGYLEVHPEFRVIFTSNPEEYAGTHKTQDALMDRMITMKIGHYDRETEIRVTRAKSGLPPSEVAIVVDIVRELRGQSVNHHRPTLRACIAIARIMADRRISARSNNSFFRDICRDILDMDSAKVRRDGNALGESPVDDVVASISARARRPKIVEPKGLHKEI

SEQ ID NO: 361 - protein, 348 aa, Tolypothrix sp
  source 1..348: note = PCC 7601
MTNTENHKKRAVLRVRPGQFVVTPAIEKVAIRALRYLTSGFAIHLRGPAGTGKTTLAMHLANCLDRPIMLIFGDDEFKSSDLIGSESGYTHKKLLDNYIHNVLKVEDELKQNWVDSRLTLACREGLTLVYDEFNRSRPEVNNVLLSALEEKILTLPPSSNQPEYLHVHPKFRAIFTSNPEEYCGVHSTQDALMDRLVTINMPEPDEQTQIEILTHKTGIHHEYAQLIARLVKAFRSATGAEKTSGLRSCLMVAKVCAEHDILVTPENTDFREICADVLFNRTNLSASDATTLFLELLNHVQVKPVEPVDDSDPYDVAEAEIVGAAEPQTDAIAEPVTLDESLLSDQPN

SEQ ID NO: 362 - protein, 311 aa, Trichodesmium erythraeum
  source 1..311: note = IMS101
MTTVLNVSPDRFVSTPGVERVTQRASRYLESGYSVHLRGPAGVGKTTLALHLAHLRQQPIFLMIGDDEFKTSDLIGNKSGYTRKKLVDNYIHTVLKVEDELRDNWIDSRLTLACKEGFTLIYDEFNRSRPEVNNVLLSVLEEKMLVLPPSQNQSEYIQVHPQFRVILTSNSEEWTGVHATQDALLDRVVTIGMEQPDISTEQNIVIQKTGINPLKAEVIIKLVRSVRQRVDKEDLGSLRSALMISKVCHDHDIPLDGKDSSFSDLCADILISRPNLPRQEALQQLDEVLEEFFPADQPSSSDVGLEKEGSL

SEQ ID NO: 363 - protein, 311 aa, Trichodesmium erythraeum
  source 1..311: note = IMS101
MTTVLNVSPDRFVSTPSVERVTQRASRYLESGYSVHLRGPAGVGKTTLALHLAHLRQQPIFLMIGDDEFKTSDLIGNKSGYTRKKLVDNYIHTVLKVEDELKHNWIDSRLTLACKEGFTLIYDEFNRSRPEVNNVLLSVLEEKMLVLPPSQNQSEYIQVHPQFRVILTSNSEEWTGVHATQDALLDRVVTIGMGQPDISTEQNIIIQKTGINPLKAEVIIKLVRSVRERLETEDLGSLRSALMISKVCHDHDIPLGGKDSNFSDLCADILISRANLPRQEALKQLDEVLEELFPADQLSISDIGLKKEGSL

SEQ ID NO: 364 - protein, 114 aa, Anabaena-flos-aquae
MIKNIQVFFMKTISNRSISRAKISTMPRPKSDASSQLDLYKMVTEKQRIQRDMYSIKERMGLLQQRLDILNQQIEATEKTIHKLRQPHSNTAQNIVRSNIFVESNNYQTFEVEY

SEQ ID NO: 365 - protein, 105 aa, Aphanizomenon flos-aquae
  source 1..105: note = NIES-81
MKSFRHRSIIRAKISTMPRHISEASSQLELYKMVAEKQRISRELSSIKERMATLQKRLDSLNNEIDNTEKTIHKLRQPHSSTAQNIVRSKNVVESNNYQTFEIEY

SEQ ID NO: 366 - protein, 91 aa, Burkholderia thailandensis
  source 1..91: note = sp. Bp5365 strain MSMB43
MPIPKKGLHDIRFRHAPGATPLPVHSMYMRISCIEMEKSRRTIERRAAQRRIAAVDSRVADLEREKARLYAAIDNEAPQAGDIRGSFRIRY

SEQ ID NO: 367 - protein, 103 aa, Desulfobacterium vacuolatum
  source 1..103: note = DSM 3385
MLKNRNRSIKGVQNIKTHAGKVDHVSHPHMAYMRISCLEMEKARKNKEKSGAQKRIDMINQRLMEIEKEKAHIQRILGDTSIALESSNVDHDSEIKGGFKIKY

SEQ ID NO: 368 - protein, 106 aa, Desulfomonile tiedjei
  source 1..106: note = DSM 6799
MNIRMKGNSRGLRDIRTHSGKVDRVGLPYMAYMSISCLEMEKARREKERLSALTRIKNIEQRIREIEAEKDLLLKGVGERTRTDLQKASTPRDQSAQCKGGFKIRY

SEQ ID NO: 369 - protein, 105 aa, Legionella drancourtii
  source 1..105: note = LLAP12
MMPALVKGLRNIKTMSNRLDKVQSPHEAFISAAALHREKQRHLQELAILRNRLDEINLRLEQINEQQNQVAEAFDISPPRAVKSALRTGIQSKTGSTSHGFKIKY

SEQ ID NO: 370 - protein, 110 aa, Microcystis aeruginosa
  source 1..110: note = NIES-843
MTTTRPPRPIRSKISTMPRKQSEADHQLELYKLITEKQRIQEKLEMMERQIQQLKNRLTFVTEQIETTEQSIQNLRTANPPSVAKKPDSPKTVAHSSNNSSNFQTFYLEY

SEQ ID NO: 371 - protein, 94 aa, Nostoc punctiforme
  source 1..94: note = ATCC 29133
MHRTPNRRQIQAKLSTMPPQRSQATVYLNAYKMMLEKERLEEELEKLEARRHQIQQRLAILNSQTIPEENMTHQQANTDLENNTPKFNTLTLEY

SEQ ID NO: 372 - protein, 113 aa, Nostoc sp
  source 1..113: note = PCC 7120
MLSIIQVFPMTKVRNRGIIRPKITTMPRNKSEASSQLELYKLVTEQQRIKQELAFIEQRTVLLKQRLSTLKTQIEGTERSINHLRHSELKYSRIALPKIFSETNNYQAFDIEY

SEQ ID NO: 373 - protein, 108 aa, Planktothrix agardhii
  source 1..108: note = str. 7805
MRPFRSQPPILPKISTMPRQKTEATLYRSLYQLAVEKKRLQEELESLGQRFETVTQRLQQIETQIQGLETDVKQIAPPKPPETKPNQPSTPTPTKAEPGSVSTFTLDY

SEQ ID NO: 374 - protein, 103 aa, Psychromonas ingrahamii
  source 1..103: note = 37
MTAAKRKTLRGLADIRTISSCGTSGQEAYQMYLKRGVLEMEKLRRQKEKNSALERVTNINRRLMAIDTDIDFLCQSLKVIEKRTNQENSIVEKSVSRGFKLRY

SEQ ID NO: 375 - protein, 106 aa, Psychromonas ingrahamii
  source 1..106: note = 37
MIFSKKKNALRGLADIRTLSGCGTSGQEAYQMYLKRGVLEMEKLRRQKEKNSALERVRNINYRLMAIDADIDFLCQSLKVIEERTNKENSISNESVTYKKGFKLRY

SEQ ID NO: 376 - protein, 105 aa, Serratia sp
  source 1..105: note = ATCC 39006
MAISTRPLRTLSDIKTHSGRVSGEHQTYRDYFQIGALELERWRRTREREAASSRIASIDERIADIDKEKAALLADATAASAVAENNDKSEAAEKKKKSSGLRIKY

SEQ ID NO: 377 - protein, 130 aa, Thiocapsa rosea
  source 1..130: note = strain DSM 235
MSKFTQPSRSVRDIKTLAGMADDVRAPHKMYMRLFALETERHRRLQERASAMLRVDNIDARCAEIAEEMEQLLQILGVEAVAPGGPPANARPGSGRVPTQPHRGRGKGTGAGRQTTSGETSVGEAVKIRY

SEQ ID NO: 378 - protein, 226 aa, Anabaena-flos-aquae
MELENLYTYAFLEIPSSPLILPQGAANQVVLINGTELAAIVEPGIFLESFQNNDEKIIQMALSHDRVICELFQQITVLPLRFGTYFTSTNNLLNHLKSHEKEYQNKLEKINGKNEFTLKLIPRMIEEIVPSEGGGKDYFLAKKQRYQNQNNFSIAQAAEKQNLIDLITKVNQLPVVVQEQEEQIQIYLLVSCQDKTLLLEQFLTWQKACPRWDLLLGDCLPPYHFI

SEQ ID NO: 379 - protein, 226 aa, Aphanizomenon flos-aquae
  source 1..226: note = NIES-81
MELENLYTYAFLKTPSFSLHLPQGSTTSVIQIDGNGLSAIVEPGISLDSFQDDDEKIVQMAIEHDRVICDIFRQITVLPLRFGTYFANTDNLLTHLESYGQEYLDKLEKINCKTEFILKLIPRMITEESPVLESGRHYFLAKKQHYQRQKNFILAQASEKEILINFISKINQIPVIIQEQEEEVRIYLLVNYQDKTLLLEQFLTWQQTCPRWDLFLGEGIPPYHFI

SEQ ID NO: 380 - protein, 227 aa, Arthrospira platensis
  source 1..227: note = NIES-39
MYVYAFIKSQSISWKSVQGIYEPVVLLEAGALAAVVEPNLQAENLSADNEEELMRAVLTHDRIVCQIFEETTVLPVRFGTCFDSEARLCEHLTTEGDRYFRQLEKLTGRAEYLLEAIPQPFNQEKPSSDTTAPPTKGRDYFLQKKRLHQQRLNFEQQQEQQWQDFINAIASKYPIVQGKATEDAERIYLLIPRSQEVALVEWVAQQQQNIDLWEFSLGNAVPAYHFL

SEQ ID NO: 381 - protein, 226 aa, Dolichospermum circinale
MKLENFYTYAFLEIPRFPLVLPQGAASQVILINGSGMSAIVEPGISLESFQNNDEKIIQMALSHDRVICELFQQVTVLPLRFGTCFTSTNNLLNYLELHRQEYQEKLEKINGKIEFTLKLIPQTMEEPAPLERGGRDYFLAKKQRYQDQNNFRIAQAAEKQNLIDSISKVNQLPFVIQEKEEEVNIYLLVKSEDKTLLLEQFLNWQKACPRWDLLLGEPLPPYHFI

SEQ ID NO: 382 - protein, 227 aa, Microcystis aeruginosa
  source 1..227: note = NIES-843
MKLYNLYTYAFLKTPIESLKLPVGMANPLLLITGGELSAVVEPEVGLDTLQNDDERLIQSVLCHDRVICQLFQQTTILPLRFGTSFLEAENLLTHLCSHGQEYQEKIEELEGKGEYLLKCIPRKPEEPVLFSESKGRQYFLAKKQLYEAQQDFYTLQGSEWQNLVNLITQSYPSTRIITAPGTESRIYLLVNLQEEPLLIEQVLHWQKACPRWELQLGQVSPPYHFT

SEQ ID NO: 383 - protein, 229 aa, Nostoc punctiforme
  source 1..229: note = ATCC 29133
MSIYAYALLVPTASPLVLPLGMERNTELVYSSGLAALVEPEISLEAIQATDERLLQAVLNHDHVIRELFQQTPLLPLRFGRGFTSVEKLLNHLENHQEQYLETLTQLADKVEYSVKVTACSLLDDSDTIDARGKAYLLAKKQRYQTQQAFQAQQCEQWELLNELILKTYTNVICETRQSDVRQIHFLAQRNDSTLSTQLFSLWQVQCSHWQLALSEPLPPYHFLKNTLI

SEQ ID NO: 384 - protein, 227 aa, Nostoc sp
  source 1..227: note = PCC 7120
MRSPNFYTYAFLNTPDIPLRLPSGNLGQLLLIHGHKLSAVVEPGISLESSQNNDEEVIKMVLAHDRVICELSQQTTVLPLRFGTYFNSEETLLNHIESHAQEYQKKLDHIQGKTEYTLKLIPRKFEELAKVSGGNGRDYFLAKKLHYEHQKNFIGDQNREKNHLINLIMDVYRSSAIIQDYVEEVRLHLLVDRHDKTLLFKQVLTLQEKCPHWNLILGEPLPPYHFV

SEQ ID NO: 385 - protein, 88 aa, Bacillus-megaterium
MEIKKIMQAVNDFFGEHVAPPHKITSVEATEDEGWRVIVEVIEEREYMKKYAKDEMLGTYECFVNKEKEVISFKRLDVRYRSAIGIEA

SEQ ID NO: 386 - protein, 95 aa, Bacillus-megaterium
MSLKQSMENKDIALIDILDVILDKGVAIKGDLIISIAGVDLVYLDLRVLISSVETLVQAKEGNHKPITSEQFDKQKEELMDATGQPSKWTNPLGS

SEQ ID NO: 387 - protein, 65 aa, Rhodococcus hoagie
  source 1..65: note = 103S
MSATPDRRIALVDLLDRVLGGGVVVAGEITLSIADVDMVHISLRTLVSSVSALTRPPDEKPENDG

SEQ ID NO: 388 - protein, 292 aa, Bacillus-megaterium
MATETKLDNTQAENKENKNAENGSKEKNGSKASKTTSSGPIKRAVAGGIIGATIGYVSTPENRKSLLDRIDTDELKSKASDLGTKVKEKSKSSVASLKTSAGSLFKKDKDKSKDDEENVNSSSSETEDDNVQEYDELKEENQTLQDRLSQLEEKMNMLVELSLNKNQDEEAEDTDSDEEENDENDENDENEQDDENEEETSKPRKKDKKEAEEEESESDEDSEEEEEDSRSNKKNKKVKTEEEDEDESEEEKKEAKPKKSTAKKSKNTKAKKNTDEEDDEATSLSSEDDTTA

SEQ ID NO: 389 - protein, 138 aa, Bacillus-megaterium
MSTGPSFSTKDNTLEYFVKASNKHGFSLDISLNVNGAVISGTMISAKEYFDYLSETFEEGSEVAQALSEQFSLASEASESNGEAEAHFIHLKNTKIYCGDSKSTPSKGKIFWRGKIAEVDGFFLGKISDAKSTSKKSS

SEQ ID NO: 390 - protein, 193 aa, Anabaena flos-aquae
MISLMAKIRQEHQSIAEKVAELSLETREFLSVTTAKRQEQAEKQAQELQAFYKDLQETSQQFLSETAQARIAQAEKQAQELLAFHKELQETSQQFLSATAQARIAQAEKQAQELLAFYQEVRETSQQFLSATAQARIAQAEKQAQELLAFHKELQETSQQFLSATADARTAQAKEQKESLLKFRQDLFVSIFG

SEQ ID NO: 391 - protein, 382 aa, Halobacterium salinarum
MSVTDKRDEMSTARDKFAESQQEFESYADEFAADITAKQDDVSDLVDAITDFQAEMTNTTDAFHTYGDEFAAEVDHLRADIDAQRDVIREMQDAFEAYADIFATDIADKQDIGNLLAAIEALRTEMNSTHGAFEAYADDFAADVAALRDISDLVAAIDDFQEEFIAVQDAFDNYAGDFDAEIDQLHAAIADQHDSFDATADAFAEYRDEFYRIEVEALLEAINDFQQDIGDFRAEFETTEDAFVAFARDFYGHEITAEEGAAEAEAEPVEADADVEAEAEVSPDEAGGESAGTEEEETEPAEVETAAPEVEGSPADTADEAEDTEAEEETEEEAPEDMVQCRVCGEYYQAITEPHLQTHDMTIQEYRDEYGEDVPLRPDDKT

SEQ ID NO: 392 - protein, 381 aa, Halobacterium mediterranei
MSVKDKREKMTATREEFAEVQQAFAAYADEFAADVDDKRDVSELVDGIDTLRTEMNSTNDAFRAYSEEFAADVEHFHTSVADRRDAFDAYADIFATDVAEMQDVSDLLAAIDDLRAEMDETHEAFDAYADAFVTDVATLRDVSDLLTAISELQSEFVSVQGEFNGYASEFGADIDQFHAVVAEKRDGHKDVADAFLQYREEFHGVEVQSLLDNIAAFQREMGDYRKAFETTEEAFASFARDFYGQGAAPMATPLNNAAETAVTGTETEVDIPPIEDSVEPDGEDEDSKADDVEAEAEVETVEMEFGAEMDTEADEDVQSESVREDDQFLDDETPEDMVQCLVCGEYYQAITEPHLQTHDMTIKKYREEYGEDVPLRPDDKA

SEQ ID NO: 393 - protein, 162 aa, Microchaete diplosiphon
MTPLMIRIRQEHRGIAEEVTQLFKDTQEFLSVTTAQRQAQAKEQAENLHQFHKDLEKDTEEFLTDTAKERMAKAKQQAEDLFQFHKEMAENTQEFLSETAKERMAQAQEQARQLREFHQNLEQTTNEFLADTAKERMAQAQEQKQQLHQFRQDLFASIFGTF

SEQ ID NO: 394 - protein, 129 aa, Nostoc sp
MTALMVRIRQEHRSIAEEVTQLFRETHEFLSATTAHRQEQAKQQAQQLHQFHQNLEQTTHEFLTETTTQRVAQAEAQANFLHKFHQNLEQTTQEFLAETAKNRTEQAKAQSQYLQQFRKDLFASIFGTF

SEQ ID NO: 395 - protein, 88 aa, Bacillus megaterium; identical to SEQ ID NO: 84

SEQ ID NO: 396 - protein, 88 aa, Bacillus megaterium; identical to SEQ ID NO: 385

SEQ ID NO: 397 - protein, 308 aa, Bacillus megaterium; identical to SEQ ID NO: 11

SEQ ID NO: 398 - protein, 255 aa, Bacillus megaterium; identical to SEQ ID NO: 12

SEQ ID NO: 399 - protein, 88 aa, Bacillus megaterium
MLHKLVTAPINLVVKIGEKVQEEADKQLYDLPTIQQKLIQLQMMFELGEIPEEAFQEKEDELLMRYEIAKRREIEQWEELTQKRNEES

SEQ ID NO: 400 - protein, 269 aa, Bacillus megaterium; identical to SEQ ID NO: 306

SEQ ID NO: 401 - protein, 95 aa, Bacillus megaterium; identical to SEQ ID NO: 386

SEQ ID NO: 402 - protein, 94 aa, Bacillus megaterium; identical to SEQ ID NO: 270

SEQ ID NO: 403 - protein, 100 aa, Bacillus megaterium; identical to SEQ ID NO: 207

SEQ ID NO: 404 - protein, 292 aa, Bacillus megaterium; identical to SEQ ID NO: 388

SEQ ID NO: 405 - protein, 138 aa, Bacillus megaterium; identical to SEQ ID NO: 389

SEQ ID NO: 406 - protein, 72 aa, Serratia sp.; identical to SEQ ID NO: 95

SEQ ID NO: 407 - protein, 65 aa, Serratia sp.; identical to SEQ ID NO: 257

SEQ ID NO: 408 - protein, 65 aa, Serratia; identical to SEQ ID NO: 257

SEQ ID NO: 409 - protein, 164 aa, Serratia sp.
MGCLTDGMAQLRKNIDDSHESRIAQQNARVSSVSAQIAGFSTTRARNAAQDARARATFVADNVRGVNRMLSDFCHTREVMSRQQSEERATFVTDMSKKTLALLDGFNAERKSMAERCAKERADFIANVANDVAAFLSASEKDRMAAHAVFFGMTLAKKKTSLAV

SEQ ID NO: 410 - protein, 329 aa, Serratia sp.; identical to SEQ ID NO: 358

SEQ ID NO: 411 - protein, 105 aa, Serratia sp.; identical to SEQ ID NO: 376

SEQ ID NO: 412 - protein, 273 aa, Serratia sp.; identical to SEQ ID NO: 140

SEQ ID NO: 413 - protein, 262 aa, Serratia sp.; identical to SEQ ID NO: 322

SEQ ID NO: 414 - protein, 222 aa, Serratia sp.; identical to SEQ ID NO: 168

SEQ ID NO: 415 - protein, 138 aa, Serratia sp.; identical to SEQ ID NO: 201

SEQ ID NO: 416 - protein, 637 aa, Serratia sp.
MKPAIYPKFLLESPLKLVFFGGKGGVGKSTCATSTALRLAQEQPQHHFLLVSTDPAHSLQNILSDLVLPKNLDVRELNAAASLHEFKSQHEGVLKEIAYRGTVLDQNDVQGLMDTALPGMDELAAYLEIAEWIQKDTYYRIIIDTAPTGHTLRLLEMPDLIYRWLTALDTLLAKQRYIRKRFAGDNRLDHLDHFLLDMNDSLKAMHELVTDSTRCCFVLVMLAEAMSVEESIDLAGALNQQRVFLSDLVVNRLFPENDCPTCCVERNRQMLALQNGYQRLPGHVFWTLPLLAIEPRGALLHEFWSGVRLLDENEVMATTCHHQLPLRVESSISLPASTFRLLIFAGKGGVGKTTLACATALRLNSEYPELRILLFSADPAHSLSDCLGVTLQQQPISVLVNIDAQEINAQADFDKIRQGYRAELEAFLLDTLPNLDITFDREVLEHLLDLAPPGLDEIMALTAIMDHLDSGRYDMVIVDGAPSGHLLRLLELPELIRDWLKQFFSLLLKYRKVMRFPHLSERLVQLSRELKNLRALLQDTKQTGLYAVTVPTHLALEKTYEMTCALQRLGLTANALFINQITPPSDCTLCQAITSRESLELKCADEMFPSQPHAQIFRQTEPTGLSKLKTLGSALFL

SEQ ID NO: 417 - protein, 115 aa, Serratia sp.; identical to SEQ ID NO: 298

SEQ ID NO: 418 - protein, 87 aa, Serratia sp.
MVNTTNDINAATRGLLLRMGNAWFEQDELRQAVDIYLKIIEQYPDSKESKTAQTALLTISQRYERDGLFRLSLDILERVGEITPTSI

SEQ ID NO: 419 - protein, 192 aa, Serratia sp.
MRALIHFPIIHSPKDLGTLSEAASHLRTETQTRAYLAAVEGFWTMITTTIEGLDLDYTHLKLYQDGLPVCGKENEIVTDVANAGSQNYKLLLTLQHKGAILMGTESPELLLQERDLMTQLLQSTEQTEASLETAKTLLNRRDDYIAQRIDETLQDGEMAILFLGLMHNIEAKLPADIVFIQPLGKPPGGESI

SEQ ID NO: 420 - protein, 160 aa, Serratia sp.
MTGNVEGILRGLGDLVEKLVETGEQIKRSGAFDIDTNDGKNAKAVYGFSIKMGLDGNQENRVEPFGNIRRDEQTGEATVQEVSEPLVDVIEESDHVLVLAEMPGVADEDVQVELNGDILTLHSERGSKKYHKEIVLPCSFDDKAMERSCRNGILEVKLGK

SEQ ID NO: 421 - protein, 720 aa, Serratia sp.
MSEELKLKVAEALPKDAGRGYARLDPADMARLNLAVGDIVQLTSKKGTGIAKLMPTYPDMRNKGIVQLDGLTRRNTSLSLDEKVQIEPASCKHATQIVLIPTTITPNQRDLDYIGSLLDGLPVQKGDLLRAHLFGSRSADFKVESTIPDGAVLIDPTTTLVIGKSNAVGNSSHSTQRLSYEDVGGLKNQVRRIREMIELPLRYPEVFERLGIDAPKGVLLSGPPGCGKTLIARIIAQETDAQFFTISGPEIVHKFYGESEAHLRKIFEEAGRKGPSIIFLDEIDSIAPHRDKVVGDVEKRIVAQLLALMDGLKNRGKVIVIAATNLPNAIDPALRRPGRFDREISIPIPDREGRREIIEIHSTGMPLNADVDLNVLADITHGFVGADLEALCREAAMSALRRLLPEIDFSSAELPYDRLAELTVMMDDFRAALCEVSPSAIRELFVDIPDVRWEDVGGLDDVRRRLIESVEWPIKYPELYEQAGVKPPKGLLLAGPPGVGKTLIAKAVANESGVNVISVKGPALMSRYVGDSEKGVRELFLKARQAAPCIIFLDEVDSVIPARNEGAIDSHVAERVLSQFLSEMDGLEELKGVFVMGATNRADLIDPAMLRPGRFDEIIELGLPDEDARRQILAVHLRNKPLGDNIHADDLAERCDGASGAELAAVCNRAALAALRRAIQQSEEAVLSPSTVGETPVALTVRIEQHDFAEVIAEMFGDDA

SEQ ID NO: 422 - protein, 71 aa, Anabaena flos-aquae; identical to SEQ ID NO: 34

SEQ ID NO: 423 - protein, 193 aa, Anabaena flos-aquae; identical to SEQ ID NO: 390

SEQ ID NO: 424 - protein, 335 aa, Anabaena flos-aquae; identical to SEQ ID NO: 326

SEQ ID NO: 425 - protein, 248 aa, Anabaena flos-aquae; identical to SEQ ID NO: 206

SEQ ID NO: 426 - protein, 155 aa, Anabaena flos-aquae; identical to SEQ ID NO: 269

SEQ ID NO: 427 - protein, 245 aa, Anabaena flos-aquae; identical to SEQ ID NO: 106

SEQ ID NO: 428 - protein, 119 aa, Anabaena flos-aquae; identical to SEQ ID NO: 172

SEQ ID NO: 429 - protein, 114 aa, Anabaena flos-aquae; identical to SEQ ID NO: 364

SEQ ID NO: 430 - protein, 226 aa, Anabaena flos-aquae
MELENLYTYAFLEIPSSPLILPQGAANQVVLINGTELAAIVEPGIFLESFQNNDEKIIQMALSHDRVICELFQQITVLPLRFGTYFTSTNNLLNHLKSHEKEYQNKLEKINGKNEFTLKLIPRMIEEIVPSEGGGKDYFLAKKQRYQNQNNFSIAQAAEKQNLIDLITKVNQLPVVVQEQEEQIQIYLLVSCQDKTLLLEQFLTWQKACPKWDLLLGDCLPPYHFI

SEQ ID NO: 431 - DNA, 267 bp, Bacillus megaterium
  source 1..267: mol_type = genomic DNA
atgagcatccagaagtccaccaacagcagcagcctggccgaagtgatcgaccggatcctggacaagggcatcgtgatcgacgccttcgccagagtgtccgtcgtgggcatcgagatcctgaccatcgaggccagagtcgtgatcgccagcgtggacacctggctgagatatgccgaagccgtgggcctgctgcgggacgacgtggaagaaaatggcctgcccgagcggagcaacagctctgagggacagccccggttcagcatctga

SEQ ID NO: 432 - DNA, 927 bp, Bacillus megaterium
  source 1..927: mol_type = genomic DNA
atgaccgtgctgaccgacaagcggaagaagggcagcggcgccttcatccaggacgacgagacaaaagaggtgctgagcagagccctgagctacctgaagtccggctacagcatccacttcaccggacctgccggcggaggcaagacatctctggctagagccctggccaagaaacggaagcggcccgtgatgctgatgcacggcaaccacgagctgaacaacaaggacctgatcggcgatttcaccggctacaccagcaagaaagtgatcgaccagtacgtgcggagcgtgtacaagaaagacgaacaggtgtccgagaactggcaggacggcagactgctggaagccgtgaagaatggctacaccctgatctacgacgagttcaccagaagcaagcccgctaccaacaacatcttcctgagcatcctggaagagggcgtgctgcccctgtacggcgtgaagatgaccgaccctttcgtgcgcgtgcaccccgacttcagagtgatcttcaccagcaaccccgccgagtatgccggcgtgtacgatacccaggacgccctgctggaccggctgatcaccatgttcatcgactacaaggacatcgaccgggaaaccgccatcctgaccgagaaaaccgacgtggaagaggacgaggcccggaccatcgtgaccctggtggccaacgtgcggaacagaagcggcgacgagaatagcagcggcctgagcctgagagccagcctgatgattgccaccctggccacccagcaggacatccctatcgatggcagcgacgaggacttccagaccctgtgcatcgacatcctgcaccaccccctgaccaagtgcctggacgaggaaaacgccaagagcaaggccgagaagatcattctggaagagtgcaagaacatcgacaccgaggaaaagtga

SEQ ID NO: 433 - DNA, 768 bp, Bacillus megaterium
  source 1..768: mol_type = genomic DNA
atgagcgagacaaacgagacaggcatctacatcttcagcgccatccagaccgacaaggacgaggaattcggcgccgtggaagtggaagggaccaaggccgagacattcctgatccggtacaaggacgccgccatggtggccgccgaagtgcccatgaagatctaccaccccaaccggcagaacctgctgatgcaccagaatgccgtggccgccatcatggacaagaacgacaccgtgatccccatcagcttcggcaacgtgttcaagagcaaagaggacgtgaaggtgctgctggaaaacctgtacccccagttcgagaagctgttccccgccatcaagggaaagatcgaagtgggcctgaaagtgatcggcaagaaagagtggctggaaaagaaagtgaacgagaaccccgagctggaaaaagtgtccgccagcgtgaagggcaagagcgaggccgctggctactacgagagaatccagctgggcggcatggcccagaagatgttcaccagcctgcagaaagaagtgaaaaccgacgtgttcagccccctggaagaagccgccgaggccgccaaagccaatgagcctacaggcgagacaatgctgctgaacgccagcttcctgatcaacagagaggacgaggccaagttcgacgaaaaagtgaatgaggcccacgagaactggaaggataaggccgacttccactacagcggcccctggcccgcctacaacttcgtgaacatccggctgaaggtggaagagaagtga

SEQ ID NO: 434 - DNA, 267 bp, Bacillus megaterium
  source 1..267: mol_type = genomic DNA
atgctgcacaagctcgtgaccgcccccatcaacctggtcgtgaagatcggcgagaaggtgcaggaagaggccgacaagcagctgtacgacctgcccaccatccagcagaagctgatccagctgcagatgatgttcgagctgggcgagatccccgaggaagccttccaggaaaaagaggacgagctgctgatgagatacgagatcgccaagcggcgcgagatcgagcagtgggaggaactgacccagaagcggaacgaggaaagctga

SEQ ID NO: 435 - DNA, 810 bp, Bacillus megaterium
  source 1..810: mol_type = genomic DNA
atgggcgagctgctgtacctgtacggcctgatccccaccaaagaggccgctgccatcgagcccttcccattctacaagggcttcgacggcgagcacagcctgtaccctatcgccttcgaccaagtgaccgccgtggtgttcaagctggacgccgacacctacagcgagaaagtgatccaggaaaagatggaacaggacatgagctggctgcaggaaaaggccttccaccaccacgagacagtggccgccctgtacgaggaattcaccatcatccccctgaagttctgcaccatctataagggcgaggaatccctgcaggccgccatcgagatcaacaaagagaagatcgagaactccctgaccctgctgcagggcaacgaggaatggaacgtgaagatctactgcgacgacaccgagctgaagaagggcatcagcgagacaaacgagagcgtgaaggccaagaagcaggaaatcagccacctgagccccggcagacagttcttcgagaagaagaagattgaccagctgatcgagaaagagctggaactgcacaagaacaaagtgtgcgaggaaatccacgacaagctgattgagctgagcctgtacgactccgtgaagaagaactggtccaaggacgtgaccggcgctgccgaacagatggcctggaacagcgtgttcctgctgcccagcctgcagatcaccaagttcgtgaacgagatcgaggaactgcagcagcggctggaaaacaagggctggaagttcgaagtgaccggcccctggcctccctaccacttcagcagctttgcctga

SEQ ID NO: 436 - DNA, 285 bp, Bacillus megaterium
  source 1..285: mol_type = genomic DNA
atgagcctgaagcagagcatggaaaacaaggatatcgccctgatcgacatcctggacgtgatcctggacaagggcgtggccatcaagggcgacctgatcatctctatcgccggcgtggacctggtgtacctggacctgagagtgctgatctccagcgtggaaaccctggtgcaggccaaagagggcaaccacaagcccatcaccagcgagcagttcgacaagcagaaagaggaactgatggacgccaccggccagcccagcaagtggacaaatcctctgggcagc

SEQ ID NO: 437 - DNA, 285 bp, Bacillus megaterium
  source 1..285: mol_type = genomic DNA
atgcagcccgtgtcccaggccaacggcagaatccacctggatcccgatcaggccgaacagggactggcccagctcgtgatgaccgtgatcgagctgctgcggcagatcgtggaacggcacgccatgagaagagtggaaggcggcaccctgaccgacgagcagatcgagaatctgggaatcgccctgatgaacctggaagagaagatggacgagctgaaagaggtgttcggactggacgccgaggacctgaacatcgacctgggccctctgggcagcctgctgtga

SEQ ID NO: 438 - DNA, 303 bp, Bacillus megaterium
  source 1..303: mol_type = genomic DNA
atggccgtggaacacaacatgcagagcagcaccatcgtggacgtgctggaaaagatcctggacaagggcgtcgtgatcgccggggacatcacagtgggaatcgccgacgtggaactgctgaccatcaagatccggctgatcgtggccagcgtggacaaggccaaagaaatcggcatggattggtgggagaacgacccctacctgagcagcaagggcgccaacaacaaggccctggaagaggaaaacaagatgctgcacgagcggctgaaaacactggaagagaagatcgagacaaagcgctga

SEQ ID NO: 439 - DNA, 417 bp, Bacillus megaterium
  source 1..417: mol_type = genomic DNA
atgagcaccggccccagcttcagcaccaaggacaacaccctggaatacttcgtgaaggccagcaacaagcacggcttcagcctggacatcagcctgaacgtgaacggggccgtgatcagcggcaccatgatcagcgccaaagagtacttcgactacctgagcgagacattcgaagagggcagcgaggtggcccaggccctgtctgagcagtttagcctggccagcgaggcctccgagtctaatggcgaagccgaggcccacttcatccacctgaagaacaccaagatctactgcggcgacagcaagagcacccccagcaagggcaagatcttctggcgcggcaagatcgccgaggtggacggattcttcctgggaaagatcagcgacgccaagtccaccagcaagaagtccagctga

SEQ ID NO: 440 - DNA, 508 bp, synthetic construct
  misc_feature 1..508: note = synthetic polynucleotide
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 441 - DNA, 135 bp, synthetic construct
  misc_feature 1..135: note = synthetic polynucleotide
  source 1..135: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 442 - protein, 24 aa, synthetic construct
  REGION 1..24: note = synthetic polypeptide
GAPGSGATNFSLLKQAGDVEENPG

SEQ ID NO: 443 - DNA, 6675 bp, synthetic construct
  misc_feature 1..6675: note = synthetic polynucleotide
  source 1..6675: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctaggcttttgcaaaaagctatttaggtgacactatagaaggtacgcctgcaggtaccgagctcggatccagtacccttcaccatgaccgtgctgaccgacaagcggaagaagggcagcggcgccttcatccaggacgacgagacaaaagaggtgctgagcagagccctgagctacctgaagtccggctacagcatccacttcaccggacctgccggcggaggcaagacatctctggctagagccctggccaagaaacggaagcggcccgtgatgctgatgcacggcaaccacgagctgaacaacaaggacctgatcggcgatttcaccggctacaccagcaaaaaggtgatcgaccagtacgtgcggagcgtgtacaagaaagacgaacaggtgtccgagaactggcaggacggcagactgctggaagccgtgaagaatggctacaccctgatctacgacgagttcaccagaagcaagcccgctaccaacaacatcttcctgagcatccttgaggagggcgtgctgcccctgtacggcgtgaagatgaccgaccctttcgtgcgcgtgcaccccgacttcagagtgatctttaccagcaaccccgccgagtatgccggcgtgtacgatacccaggacgccctgctggaccggctgatcaccatgttcatcgactacaaggacatcgaccgggaaaccgctatcctgaccgagaaaactgacgtggaagaagacgaggcccggaccatcgtgaccctggtggccaacgtgcggaacagaagcggcgacgagaatagcagcggcctgagcctgagagccagcctgatgattgccaccctggccacccagcaggacatccctatcgatggcagcgacgaggacttccagaccctgtgcatcgacatcctgcaccaccccctgaccaagtgcctggacgaagagaacgccaagagcaaggccgagaagatcattctcgaagagtgcaagaacatcgacaccgaggagaagggtgccccgggatctggcgcaacaaattttagtcttttaaagcaggcaggagacgtcgaggaaaaccctggacccgtgagcgagacaaacgagacaggcatctacatcttcagcgccatccagacagacaaggatgaggaattcggcgccgtggaagtggaagggaccaaggctgagacattcctgatccggtataaggacgccgccatggtggccgccgaagtgcccatgaagatctaccaccccaaccggcagaacctgctgatgcaccagaatgccgtggccgccatcatggacaagaacgacaccgtgatccccatcagcttcggcaacgtgttcaagagcaaagaggacgtgaaggtgctcctggaaaacctgtacccccagttcgagaagctgttccccgccatcaagggaaagatcgaagtgggcctgaaggtgatcggcaagaaagagtggctcgaaaagaaagtgaacgagaaccccgagctggaaaaagtgtccgccagcgtgaagggcaagagcgaggccgctggctactacgagagaatccagctgggcggcatggcccagaagatgttcacaagcctgcagaaagaagtgaaaaccgacgtgttcagccccctggaagaagccgccgaggccgccaaagccaatgagcctacaggcgaaacaatgctgctgaacgccagcttcctgatcaacagagaggatgaggccaagttcgacgagaaagtcaatgaggcccacgagaactggaaggataaggccgacttccactacagcggcccctggcccgcctacaacttcgtgaacatccggctgaaggtggaagagaagggggcacctggctcgggagcgaccaacttctcattactcaaacaagccggagacgttgaggagaatccaggccctgtgctgcacaagctcgtgaccgcccccatcaacctggtcgtgaagatcggcgagaaggtgcaggaagaggccgacaagcagctgtacgacctgcccaccatccagcagaagctgatccagctgcagatgatgttcgagctgggcgagatccccgaggaagccttccaggaaaaagaggacgaactgctgatgagatacgagatcgccaagcggcgcgagattgagcagtgggaagaactgacccagaagcggaatgaggaaagcggtgccccgggatctggcgcaacaaattttagtcttttaaagcaggcaggagacgtcgaggaaaaccctggacccgtgggcgagctgctgtacctctacggcctgatccccaccaaagaggccgctgctatcgagcccttcccattctacaagggcttcgacggcgagcacagcctgtaccctatcgccttcgaccaagtgaccgccgtggtgttcaagctggacgccgacacctacagcgagaaagtgatccaggaaaagatggaacaggacatgagctggctgcaggaaaaggccttccaccaccacgagacagtggccgccctgtatgaggaattcaccatcatccccctgaagttctgcaccatctataagggagaggaatccctgcaggccgccatcgagatcaacaaagagaagatcgaaaactccctgaccctgctgcagggcaacgaggaatggaacgtgaagatctactgcgacgacaccgagctgaagaagggcatcagcgagacaaacgagagcgtgaaggccaagaagcaggaaatcagccacctgagccccggcagacagttcttcgagaagaagaagattgaccagctcatcgagaaagagctggaactgcacaagaacaaagtgtgcgaggaaatccacgacaagctgattgagctgagcctctacgactccgtgaagaagaactggtccaaggacgtgacaggcgctgccgaacagatggcctggaacagcgtgttcctgctgcccagcctgcagatcaccaagttcgtgaacgagatcgaggaactccagcagcggctggagaacaagggatggaagttcgaagtgaccggcccctggcctccctaccacttcagcagctttgccggggcacctggctcgggagcgaccaacttctcattactcaaacaagccggagacgttgaggagaatccaggccctgtgagcctgaagcagagcatggagaataaggatatcgccctgatcgacatcctcgacgtgatcctggacaagggagtggccatcaagggcgacctgatcatctctatcgccggcgtggacctggtgtacctggatctgagagtgctgatctccagcgtggaaaccctggtgcaggccaaagagggcaaccacaagcccatcaccagcgagcagttcgacaagcagaaagaggagctgatggacgccaccggccagcccagcaagtggacaaatcctctgggcagcggcgctcccgggtcaggtgccacgaatttttcgttgttgaagcaagctggggatgttgaagagaacccagggcctgtgcagcccgtgtcccaggccaacggcagaatccacctggatcccgatcaggccgaacagggactggcccagctcgtgatgaccgtgatcgagctgctgcggcagatcgtggaacggcacgccatgagaagagtggaaggcggcaccctgaccgacgagcagatcgagaatctgggaatcgctctgatgaacctggaggagaagatggacgagctgaaagaggtgttcggactggacgctgaggatctgaacatcgacctgggccctctgggcagcctgctgggtgccccgggatctggcgcaacaaattttagtcttttaaagcaggcaggagacgtcgaggaaaaccctggacccgtggccgtggaacacaacatgcagagcagcaccatcgtggacgtgctggaaaagatcctggacaagggcgtcgtgatcgccggggacatcacagtgggaatcgccgacgtggaactgctgaccatcaagatccggctgatcgtggccagcgtggacaaggccaaagaaatcggcatggattggtgggagaacgacccctacctgagcagcaagggcgccaacaacaaggctctggaagaggaaaacaagatgctgcacgagcggctgaaaacactggaagagaagatcgagacaaagcgcggggcacctggctcgggagcgaccaacttctcattactcaaacaagccggagacgttgaggagaatccaggccctgtgagcaccggccccagcttcagcaccaaggacaacaccctggaatacttcgtgaaggccagcaacaagcacggctttagcctcgacatcagcctgaacgtgaatggggccgtgattagcggcaccatgatcagcgccaaagagtacttcgactacctgagcgagacattcgaagagggcagcgaagtggcccaggccctgtctgagcagtttagcctggctagcgaggcctccgagtctaatggcgaagccgaggcccacttcatccacctgaagaacaccaagatctactgcggcgacagcaagagcacccccagcaagggcaagatcttctggcgcggcaagatcgccgaggtggacggattcttcctgggaaaaatcagcgacgccaagtccaccagcaagaagtccagcggcgctcccgggtcaggtgccacgaatttttcgttgttgaagcaagctggggatgttgaagagaacccagggcctgtggtgtccaagggcgaggaactgttcaccggcgtggtgcccatcctggtggaactggatggcgacgtgaacggccacaagttcagcgtgtccggcgagggcgaaggcgacgccacatacggaaagctgaccctgaagttcatctgcaccaccggcaagctgcccgtgccttggcctaccctcgtgaccacactgacctacggcgtgcagtgcttcgccagataccccgaccacatgaagcagcacgatttcttcaagagcgccatgcccgagggctacgtgcaggaacggaccatcttcttcaaggacgacggcaactacaagacaagagccgaagtgaagttcgagggcgacaccctcgtgaaccggatcgagctgaagggcatcgacttcaaagaggatggcaacatcctgggccacaagctggagtacaactacaacagccacaaggtgtacatcaccgccgacaagcagaaaaacggcatcaaagtgaacttcaagacccggcacaacatcgaggacggcagcgtgcagctggccgaccactaccagcagaacacccccatcggagatggccccgtgctgctgcccgacaaccactacctgagcacacaaagcgccctgagcaaggaccccaacgagaagcgggaccacatggtgctgctggaatttgtgaccgccgctggcatcaccctgggcatggacgagctgtacaagtgactcgagtctagagggccccgtggctgtaatctagaggatccctcgaggggcccaagcttacgcgtgcatgcgacgtcatagctctctccctatagtgagtcgtattataagctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 444 - DNA, 4009 bp, synthetic construct
  misc_feature 1..4009: note = synthetic polynucleotide
  source 1..4009: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctaggcttttgcaaaaagctatttaggtgacactatagaaggtacgcctgcaggtaccgagctcggatccagtacccttcaccatggccgtggaacacaacatgcagagcagcaccatcgtggacgtgctggaaaagatcctggacaagggcgtcgtgatcgccggggacatcacagtgggaatcgccgacgtggaactgctgaccatcaagatccggctgatcgtggccagcgtggacaaggccaaagaaatcggcatggattggtgggagaacgacccctacctgagcagcaagggcgccaacaacaaggccctggaagaggaaaacaagatgctgcacgagcggctgaaaacactggaagagaagatcgagacaaagcgcggtgccccgggatctggcgcaacaaattttagtcttttaaagcaggcaggagacgtcgaggaaaaccctggacccgtgagcgagacaaacgagacaggcatctacatcttcagcgccatccagacagacaaggatgaggaattcggcgccgtggaagtggaagggaccaaggctgagacattcctgatccggtataaggacgccgccatggtggccgccgaagtgcccatgaagatctaccaccccaaccggcagaacctgctgatgcaccagaatgccgtggccgccatcatggacaagaacgacaccgtgatccccatcagcttcggcaacgtgttcaagagcaaagaggacgtgaaggtgctcctggaaaacctgtacccccagttcgagaagctgttccccgccatcaagggaaagatcgaagtgggcctgaaggtgatcggcaagaaagagtggctcgaaaagaaagtgaacgagaaccccgagctggaaaaagtgtccgccagcgtgaagggcaagagcgaggccgctggctactacgagagaatccagctgggcggcatggcccagaagatgttcacaagcctgcagaaagaagtgaaaaccgacgtgttcagccccctggaagaagccgccgaggccgccaaagccaatgagcctacaggcgaaacaatgctgctgaacgccagcttcctgatcaacagagaggatgaggccaagttcgacgagaaagtcaatgaggcccacgagaactggaaggataaggccgacttccactacagcggcccctggcccgcctacaacttcgtgaacatccggctgaaggtggaagagaagggggcacctggctcgggagcgaccaacttctcattactcaaacaagccggagacgttgaggagaatccaggccctgtgctgcacaagctcgtgaccgcccccatcaacctggtcgtgaagatcggcgagaaggtgcaggaagaggccgacaagcagctgtacgacctgcccaccatccagcagaagctgatccagctgcagatgatgttcgagctgggcgagatccccgaggaagccttccaggaaaaagaggacgaactgctgatgagatacgagatcgccaagcggcgcgagattgagcagtgggaagaactgacccagaagcggaatgaggaaagcggtgccccgggatctggcgcaacaaattttagtcttttaaagcaggcaggagacgtcgaggaaaaccctggacccgtgggcgagctgctgtacctctacggcctgatccccaccaaagaggccgctgctatcgagcccttcccattctacaagggcttcgacggcgagcacagcctgtaccctatcgccttcgaccaagtgaccgccgtggtgttcaagctggacgccgacacctacagcgagaaagtgatccaggaaaagatggaacaggacatgagctggctgcaggaaaaggccttccaccaccacgagacagtggccgccctgtatgaggaattcaccatcatccccctgaagttctgcaccatctataagggagaggaatccctgcaggccgccatcgagatcaacaaagagaagatcgaaaactccctgaccctgctgcagggcaacgaggaatggaacgtgaagatctactgcgacgacaccgagctgaagaagggcatcagcgagacaaacgagagcgtgaaggccaagaagcaggaaatcagccacctgagccccggcagacagttcttcgagaagaagaagattgaccagctcatcgagaaagagctggaactgcacaagaacaaagtgtgcgaggaaatccacgacaagctgattgagctgagcctctacgactccgtgaagaagaactggtccaaggacgtgacaggcgctgccgaacagatggcctggaacagcgtgttcctgctgcccagcctgcagatcaccaagttcgtgaacgagatcgaggaactccagcagcggctggagaacaagggatggaagttcgaagtgaccggcccctggcctccctaccacttcagcagctttgccggggcacctggctcgggagcgaccaacttctcattactcaaacaagccggagacgttgaggagaatccaggccctgtgcagcccgtgtcccaggccaacggcagaatccacctggatcccgatcaggccgaacagggactggcccagctcgtgatgaccgtgatcgagctgctgcggcagatcgtggaacggcacgccatgagaagagtggaaggcggcaccctgaccgacgagcagatcgagaatctgggaatcgccctgatgaacctggaagagaagatggacgagctgaaagaggtgttcggactggacgccgaggacctgaacatcgacctgggccctctgggcagcctgctgtgataatctagaggatccctcgaggggcccaagcttacgcgtgcatgcgacgtcatagctctctccctatagtgagtcgtattataagctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 445 - DNA, 3371 bp, synthetic construct
  misc_feature 1..3371: note = synthetic polynucleotide
  source 1..3371: mol_type = other DNA
ccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtttcactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttggtacctatgcatgccaccatgagcatccagaagtccaccaacagcagcagcctggccgaagtgatcgaccggatcctggacaagggcatcgtgatcgacgccttcgccagagtgtccgtcgtgggcatcgagatcctgaccatcgaggccagagtcgtgatcgccagcgtggacacctggctgagatatgccgaagccgtgggcctgctgcgggacgacgtggaagaaaatggcctgcccgagcggagcaacagctctgagggacagccccggttcagcatctgaactaaatcgcactgtcggcgtccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaactagttcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 446 - DNA, 7161 bp, synthetic construct
  misc_feature 1..7161: note = synthetic polynucleotide
  source 1..7161: mol_type = other DNA
ccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtttcactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttggtacctatgcatgccaccatgaccgtgctgaccgacaagcggaagaagggcagcggcgccttcatccaggacgacgagacaaaagaggtgctgagcagagccctgagctacctgaagtccggctacagcatccacttcaccggacctgccggcggaggcaagacatctctggctagagccctggccaagaaacggaagcggcccgtgatgctgatgcacggcaaccacgagctgaacaacaaggacctgatcggcgatttcaccggctacaccagcaaaaaggtgatcgaccagtacgtgcggagcgtgtacaagaaagacgaacaggtgtccgagaactggcaggacggcagactgctggaagccgtgaagaatggctacaccctgatctacgacgagttcaccagaagcaagcccgctaccaacaacatcttcctgagcatccttgaggagggcgtgctgcccctgtacggcgtgaagatgaccgaccctttcgtgcgcgtgcaccccgacttcagagtgatctttaccagcaaccccgccgagtatgccggcgtgtacgatacccaggacgccctgctggaccggctgatcaccatgttcatcgactacaaggacatcgaccgggaaaccgctatcctgaccgagaaaactgacgtggaagaagacgaggcccggaccatcgtgaccctggtggccaacgtgcggaacagaagcggcgacgagaatagcagcggcctgagcctgagagccagcctgatgattgccaccctggccacccagcaggacatccctatcgatggcagcgacgaggacttccagaccctgtgcatcgacatcctgcaccaccccctgaccaagtgcctggacgaagagaacgccaagagcaaggccgagaagatcattctcgaagagtgcaagaacatcgacaccgaggagaagggtgccccgggatctggcgcaacaaattttagtcttttaaagcaggcaggagacgtcgaggaaaaccctggacccgtgagcgagacaaacgagacaggcatctacatcttcagcgccatccagacagacaaggatgaggaattcggcgccgtggaagtggaagggaccaaggctgagacattcctgatccggtataaggacgccgccatggtggccgccgaagtgcccatgaagatctaccaccccaaccggcagaacctgctgatgcaccagaatgccgtggccgccatcatggacaagaacgacaccgtgatccccatcagcttcggcaacgtgttcaagagcaaagaggacgtgaaggtgctcctggaaaacctgtacccccagttcgagaagctgttccccgccatcaagggaaagatcgaagtgggcctgaaggtgatcggcaagaaagagtggctcgaaaagaaagtgaacgagaaccccgagctggaaaaagtgtccgccagcgtgaagggcaagagcgaggccgctggctactacgagagaatccagctgggcggcatggcccagaagatgttcacaagcctgcagaaagaagtgaaaaccgacgtgttcagccccctggaagaagccgccgaggccgccaaagccaatgagcctacaggcgaaacaatgctgctgaacgccagcttcctgatcaacagagaggatgaggccaagttcgacgagaaagtcaatgaggcccacgagaactggaaggataaggccgacttccactacagcggcccctggcccgcctacaacttcgtgaacatccggctgaaggtggaagagaagggggcacctggctcgggagcgaccaacttctcattactcaaacaagccggagacgttgaggagaatccaggccctgtgctgcacaagctcgtgaccgcccccatcaacctggtcgtgaagatcggcgagaaggtgcaggaagaggccgacaagcagctgtacgacctgcccaccatccagcagaagctgatccagctgcagatgatgttcgagctgggcgagatccccgaggaagccttccaggaaaaagaggacgaactgctgatgagatacgagatcgccaagcggcgcgagattgagcagtgggaagaactgacccagaagcggaatgaggaaagcggtgccccgggatctggcgcaacaaattttagtcttttaaagcaggcaggagacgtcgaggaaaaccctggacccgtgggcgagctgctgtacctctacggcctgatccccaccaaagaggccgctgctatcgagcccttcccattctacaagggcttcgacggcgagcacagcctgtaccctatcgccttcgaccaagtgaccgccgtggtgttcaagctggacgccgacacctacagcgagaaagtgatccaggaaaagatggaacaggacatgagctggctgcaggaaaaggccttccaccaccacgagacagtggccgccctgtatgaggaattcaccatcatccccctgaagttctgcaccatctataagggagaggaatccctgcaggccgccatcgagatcaacaaagagaagatcgaaaactccctgaccctgctgcagggcaacgaggaatggaacgtgaagatctactgcgacgacaccgagctgaagaagggcatcagcgagacaaacgagagcgtgaaggccaagaagcaggaaatcagccacctgagccccggcagacagttcttcgagaagaagaagattgaccagctcatcgagaaagagctggaactgcacaagaacaaagtgtgcgaggaaatccacgacaagctgattgagctgagcctctacgactccgtgaagaagaactggtccaaggacgtgacaggcgctgccgaacagatggcctggaacagcgtgttcctgctgcccagcctgcagatcaccaagttcgtgaacgagatcgaggaactccagcagcggctggagaacaagggatggaagttcgaagtgaccggcccctggcctccctaccacttcagcagctttgccggggcacctggctcgggagcgaccaacttctcattactcaaacaagccggagacgttgaggagaatccaggccctgtgagcctgaagcagagcatggagaataaggatatcgccctgatcgacatcctcgacgtgatcctggacaagggagtggccatcaagggcgacctgatcatctctatcgccggcgtggacctggtgtacctggatctgagagtgctgatctccagcgtggaaaccctggtgcaggccaaagagggcaaccacaagcccatcaccagcgagcagttcgacaagcagaaagaggagctgatggacgccaccggccagcccagcaagtggacaaatcctctgggcagcggcgctcccgggtcaggtgccacgaatttttcgttgttgaagcaagctggggatgttgaagagaacccagggcctgtgcagcccgtgtcccaggccaacggcagaatccacctggatcccgatcaggccgaacagggactggcccagctcgtgatgaccgtgatcgagctgctgcggcagatcgtggaacggcacgccatgagaagagtggaaggcggcaccctgaccgacgagcagatcgagaatctgggaatcgctctgatgaacctggaggagaagatggacgagctgaaagaggtgttcggactggacgctgaggatctgaacatcgacctgggccctctgggcagcctgctgggtgccccgggatctggcgcaacaaattttagtcttttaaagcaggcaggagacgtcgaggaaaaccctggacccgtggccgtggaacacaacatgcagagcagcaccatcgtggacgtgctggaaaagatcctggacaagggcgtcgtgatcgccggggacatcacagtgggaatcgccgacgtggaactgctgaccatcaagatccggctgatcgtggccagcgtggacaaggccaaagaaatcggcatggattggtgggagaacgacccctacctgagcagcaagggcgccaacaacaaggctctggaagaggaaaacaagatgctgcacgagcggctgaaaacactggaagagaagatcgagacaaagcgcggggcacctggctcgggagcgaccaacttctcattactcaaacaagccggagacgttgaggagaatccaggccctgtgagcaccggccccagcttcagcaccaaggacaacaccctggaatacttcgtgaaggccagcaacaagcacggctttagcctcgacatcagcctgaacgtgaatggggccgtgattagcggcaccatgatcagcgccaaagagtacttcgactacctgagcgagacattcgaagagggcagcgaagtggcccaggccctgtctgagcagtttagcctggctagcgaggcctccgagtctaatggcgaagccgaggcccacttcatccacctgaagaacaccaagatctactgcggcgacagcaagagcacccccagcaagggcaagatcttctggcgcggcaagatcgccgaggtggacggattcttcctgggaaaaatcagcgacgccaagtccaccagcaagaagtccagcggcgctcccgggtcaggtgccacgaatttttcgttgttgaagcaagctggggatgttgaagagaacccagggcctgtggtgtccaagggcgaggaactgttcaccggcgtggtgcccatcctggtggaactggatggcgacgtgaacggccacaagttcagcgtgtccggcgagggcgaaggcgacgccacatacggaaagctgaccctgaagttcatctgcaccaccggcaagctgcccgtgccttggcctaccctcgtgaccacactgacctacggcgtgcagtgcttcgccagataccccgaccacatgaagcagcacgatttcttcaagagcgccatgcccgagggctacgtgcaggaacggaccatcttcttcaaggacgacggcaactacaagacaagagccgaagtgaagttcgagggcgacaccctcgtgaaccggatcgagctgaagggcatcgacttcaaagaggatggcaacatcctgggccacaagctggagtacaactacaacagccacaaggtgtacatcaccgccgacaagcagaaaaacggcatcaaagtgaacttcaagacccggcacaacatcgaggacggcagcgtgcagctggccgaccactaccagcagaacacccccatcggagatggccccgtgctgctgcccgacaaccactacctgagcacacaaagcgccctgagcaaggaccccaacgagaagcgggaccacatggtgctgctggaatttgtgaccgccgctggcatcaccctgggcatggacgagctgtacaagtgaactagttcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 447 - DNA, 5617 bp, synthetic construct
  misc_feature 1..5617: note = synthetic polynucleotide
  source 1..5617: mol_type = other DNA
ccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagttttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagacatatgctgcagtcacttgtacagctcatccatgcccagggtgatgccagcggcggtccgaaattccagcagcaccatgtggtcccgcttctcgttggggtccttgctcagcacgctctgggtgctcaggtagtggctatcaggcagcagcacggggccatctccgatgggggtgttctgctggtagtggtcggccagctgcacgctgccatcttccacgttgtgccggatcttgaagttcactttgatgccgtttttctgcttcacggccatgatgtagatgttgtggctgttgaagttgtactccagcttgtggcccaggatgttgccgtcctctttgaagtccacgcccttcagctcgatccggttcacgagggtgtcgccctcgaacttcacttcggctctggtcttgtaggtgccgtcgtccttgaagaagatggtccgttcctgcacgtagccctcgggcatggcgctcttgaagaaatcgtgctgcttcatgtggtcggggtatctggcgaagcactgcacgccgtgagacagtgtggtcacgagggtaggccaaggcacgggcagcttgccggtggtgcagatgaacttcagggtcagcttgccatttgtggcgtcgccttcgccctctccccgcacagagaacttgtggccgttcacgtcgccatccagttccaccaggatgggcaccacgccggtgaacagttcctcgcccttggacaccatggtgaagggtactggatccgagctcggtacctgcaggcgtaccttctatagtgtcacctaaatgcgatctgacggttcactaaacgagctctgcttatataggcctcccaccgtacacgccacctcgacatactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttggtacctatgcatgccaccatggccgtggaacacaacatgcagagcagcaccatcgtggacgtgctggaaaagatcctggacaagggcgtcgtgatcgccggggacatcacagtgggaatcgccgacgtggaactgctgaccatcaagatccggctgatcgtggccagcgtggacaaggccaaagaaatcggcatggattggtgggagaacgacccctacctgagcagcaagggcgccaacaacaaggccctggaagaggaaaacaagatgctgcacgagcggctgaaaacactggaagagaagatcgagacaaagcgcggtgccccgggatctggcgcaacaaattttagtcttttaaagcaggcaggagacgtcgaggaaaaccctggacccgtgagcgagacaaacgagacaggcatctacatcttcagcgccatccagacagacaaggatgaggaattcggcgccgtggaagtggaagggaccaaggctgagacattcctgatccggtataaggacgccgccatggtggccgccgaagtgcccatgaagatctaccaccccaaccggcagaacctgctgatgcaccagaatgccgtggccgccatcatggacaagaacgacaccgtgatccccatcagcttcggcaacgtgttcaagagcaaagaggacgtgaaggtgctcctggaaaacctgtacccccagttcgagaagctgttccccgccatcaagggaaagatcgaagtgggcctgaaggtgatcggcaagaaagagtggctcgaaaagaaagtgaacgagaaccccgagctggaaaaagtgtccgccagcgtgaagggcaagagcgaggccgctggctactacgagagaatccagctgggcggcatggcccagaagatgttcacaagcctgcagaaagaagtgaaaaccgacgtgttcagccccctggaagaagccgccgaggccgccaaagccaatgagcctacaggcgaaacaatgctgctgaacgccagcttcctgatcaacagagaggatgaggccaagttcgacgagaaagtcaatgaggcccacgagaactggaaggataaggccgacttccactacagcggcccctggcccgcctacaacttcgtgaacatccggctgaaggtggaagagaagggggcacctggctcgggagcgaccaacttctcattactcaaacaagccggagacgttgaggagaatccaggccctgtgctgcacaagctcgtgaccgcccccatcaacctggtcgtgaagatcggcgagaaggtgcaggaagaggccgacaagcagctgtacgacctgcccaccatccagcagaagctgatccagctgcagatgatgttcgagctgggcgagatccccgaggaagccttccaggaaaaagaggacgaactgctgatgagatacgagatcgccaagcggcgcgagattgagcagtgggaagaactgacccagaagcggaatgaggaaagcggtgccccgggatctggcgcaacaaattttagtcttttaaagcaggcaggagacgtcgaggaaaaccctggacccgtgggcgagctgctgtacctctacggcctgatccccaccaaagaggccgctgctatcgagcccttcccattctacaagggcttcgacggcgagcacagcctgtaccctatcgccttcgaccaagtgaccgccgtggtgttcaagctggacgccgacacctacagcgagaaagtgatccaggaaaagatggaacaggacatgagctggctgcaggaaaaggccttccaccaccacgagacagtggccgccctgtatgaggaattcaccatcatccccctgaagttctgcaccatctataagggagaggaatccctgcaggccgccatcgagatcaacaaagagaagatcgaaaactccctgaccctgctgcagggcaacgaggaatggaacgtgaagatctactgcgacgacaccgagctgaagaagggcatcagcgagacaaacgagagcgtgaaggccaagaagcaggaaatcagccacctgagccccggcagacagttcttcgagaagaagaagattgaccagctcatcgagaaagagctggaactgcacaagaacaaagtgtgcgaggaaatccacgacaagctgattgagctgagcctctacgactccgtgaagaagaactggtccaaggacgtgacaggcgctgccgaacagatggcctggaacagcgtgttcctgctgcccagcctgcagatcaccaagttcgtgaacgagatcgaggaactccagcagcggctggagaacaagggatggaagttcgaagtgaccggcccctggcctccctaccacttcagcagctttgccggggcacctggctcgggagcgaccaacttctcattactcaaacaagccggagacgttgaggagaatccaggccctgtgcagcccgtgtcccaggccaacggcagaatccacctggatcccgatcaggccgaacagggactggcccagctcgtgatgaccgtgatcgagctgctgcggcagatcgtggaacggcacgccatgagaagagtggaaggcggcaccctgaccgacgagcagatcgagaatctgggaatcgccctgatgaacctggaagagaagatggacgagctgaaagaggtgttcggactggacgccgaggacctgaacatcgacctgggccctctgggcagcctgctgtgaactagttcgataccgtcgaccgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 448 - DNA, 237 bp, synthetic construct
  misc_feature 1..237: note = synthetic polynucleotide
  source 1..237: mol_type = other DNA
ccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaactttta

SEQ ID NO: 449 - DNA, 232 bp, synthetic construct
  misc_feature 1..232: note = synthetic polynucleotide
  source 1..232: mol_type = other DNA
gagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaa

SEQ ID NO: 450 - DNA, 350 bp, synthetic construct
  misc_feature 1..350: note = synthetic polynucleotide
  source 1..350: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatactt

SEQ ID NO: 451 - DNA, 122 bp, synthetic construct
  misc_feature 1..122: note = synthetic polynucleotide
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 452 - DNA, 231 bp, synthetic construct
  misc_feature 1..231: note = synthetic polynucleotide
  source 1..231: mol_type = other DNA
ttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccct

SEQ ID NO: 453 - DNA, 309 bp, synthetic construct
  misc_feature 1..309: note = synthetic polynucleotide
  source 1..309: mol_type = other DNA
gatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 454 - DNA, 7535 bp, synthetic construct
  misc_feature 1..7535: note = synthetic polynucleotide
  source 1..7535: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctaggcttttgcaaaaagctatttaggtgacactatagaaggtacgcctgcaggtaccgagctcggatccagtacccttcaccatgagcatccagaagtccaccaacagcagcagcctggccgaagtgatcgaccggatcctggacaagggcatcgtgatcgacgccttcgccagagtgtccgtcgtgggcatcgagatcctgaccatcgaggccagagtcgtgatcgccagcgtggacacctggctgagatatgccgaagccgtgggcctgctgcgggacgacgtggaagaaaatggcctgcccgagcggagcaacagctctgagggacagccccggttcagcatctgaactaaatcgcactgtcggcgtccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccgtgaccgtgctgaccgacaagcggaagaagggcagcggcgccttcatccaggacgacgagacaaaagaggtgctgagcagagccctgagctacctgaagtccggctacagcatccacttcaccggacctgccggcggaggcaagacatctctggctagagccctggccaagaaacggaagcggcccgtgatgctgatgcacggcaaccacgagctgaacaacaaggacctgatcggcgatttcaccggctacaccagcaaaaaggtgatcgaccagtacgtgcggagcgtgtacaagaaagacgaacaggtgtccgagaactggcaggacggcagactgctggaagccgtgaagaatggctacaccctgatctacgacgagttcaccagaagcaagcccgctaccaacaacatcttcctgagcatccttgaggagggcgtgctgcccctgtacggcgtgaagatgaccgaccctttcgtgcgcgtgcaccccgacttcagagtgatctttaccagcaaccccgccgagtatgccggcgtgtacgatacccaggacgccctgctggaccggctgatcaccatgttcatcgactacaaggacatcgaccgggaaaccgctatcctgaccgagaaaactgacgtggaagaagacgaggcccggaccatcgtgaccctggtggccaacgtgcggaacagaagcggcgacgagaatagcagcggcctgagcctgagagccagcctgatgattgccaccctggccacccagcaggacatccctatcgatggcagcgacgaggacttccagaccctgtgcatcgacatcctgcaccaccccctgaccaagtgcctggacgaagagaacgccaagagcaaggccgagaagatcattctcgaagagtgcaagaacatcgacaccgaggagaagggtgccccgggatctggcgcaacaaattttagtcttttaaagcaggcaggagacgtcgaggaaaaccctggacccgtgagcgagacaaacgagacaggcatctacatcttcagcgccatccagacagacaaggatgaggaattcggcgccgtggaagtggaagggaccaaggctgagacattcctgatccggtataaggacgccgccatggtggccgccgaagtgcccatgaagatctaccaccccaaccggcagaacctgctgatgcaccagaatgccgtggccgccatcatggacaagaacgacaccgtgatccccatcagcttcggcaacgtgttcaagagcaaagaggacgtgaaggtgctcctggaaaacctgtacccccagttcgagaagctgttccccgccatcaagggaaagatcgaagtgggcctgaaggtgatcggcaagaaagagtggctcgaaaagaaagtgaacgagaaccccgagctggaaaaagtgtccgccagcgtgaagggcaagagcgaggccgctggctactacgagagaatccagctgggcggcatggcccagaagatgttcacaagcctgcagaaagaagtgaaaaccgacgtgttcagccccctggaagaagccgccgaggccgccaaagccaatgagcctacaggcgaaacaatgctgctgaacgccagcttcctgatcaacagagaggatgaggccaagttcgacgagaaagtcaatgaggcccacgagaactggaaggataaggccgacttccactacagcggcccctggcccgcctacaacttcgtgaacatccggctgaaggtggaagagaagggggcacctggctcgggagcgaccaacttctcattactcaaacaagccggagacgttgaggagaatccaggccctgtgctgcacaagctcgtgaccgcccccatcaacctggtcgtgaagatcggcgagaaggtgcaggaagaggccgacaagcagctgtacgacctgcccaccatccagcagaagctgatccagctgcagatgatgttcgagctgggcgagatccccgaggaagccttccaggaaaaagaggacgaactgctgatgagatacgagatcgccaagcggcgcgagattgagcagtgggaagaactgacccagaagcggaatgaggaaagcggtgccccgggatctggcgcaacaaattttagtcttttaaagcaggcaggagacgtcgaggaaaaccctggacccgtgggcgagctgctgtacctctacggcctgatccccaccaaagaggccgctgctatcgagcccttcccattctacaagggcttcgacggcgagcacagcctgtaccctatcgccttcgaccaagtgaccgccgtggtgttcaagctggacgccgacacctacagcgagaaagtgatccaggaaaagatggaacaggacatgagctggctgcaggaaaaggccttccaccaccacgagacagtggccgccctgtatgaggaattcaccatcatccccctgaagttctgcaccatctataagggagaggaatccctgcaggccgccatcgagatcaacaaagagaagatcgaaaactccctgaccctgctgcagggcaacgaggaatggaacgtgaagatctactgcgacgacaccgagctgaagaagggcatcagcgagacaaacgagagcgtgaaggccaagaagcaggaaatcagccacctgagccccggcagacagttcttcgagaagaagaagattgaccagctcatcgagaaagagctggaactgcacaagaacaaagtgtgcgaggaaatccacgacaagctgattgagctgagcctctacgactccgtgaagaagaactggtccaaggacgtgacaggcgctgccgaacagatggcctggaacagcgtgttcctgctgcccagcctgcagatcaccaagttcgtgaacgagatcgaggaactccagcagcggctggagaacaagggatggaagttcgaagtgaccggcccctggcctccctaccacttcagcagctttgccggggcacctggctcgggagcgaccaacttctcattactcaaacaagccggagacgttgaggagaatccaggccctgtgagcctgaagcagagcatggagaataaggatatcgccctgatcgacatcctcgacgtgatcctggacaagggagtggccatcaagggcgacctgatcatctctatcgccggcgtggacctggtgtacctggatctgagagtgctgatctccagcgtggaaaccctggtgcaggccaaagagggcaaccacaagcccatcaccagcgagcagttcgacaagcagaaagaggagctgatggacgccaccggccagcccagcaagtggacaaatcctctgggcagcggcgctcccgggtcaggtgccacgaatttttcgttgttgaagcaagctggggatgttgaagagaacccagggcctgtgcagcccgtgtcccaggccaacggcagaatccacctggatcccgatcaggccgaacagggactggcccagctcgtgatgaccgtgatcgagctgctgcggcagatcgtggaacggcacgccatgagaagagtggaaggcggcaccctgaccgacgagcagatcgagaatctgggaatcgctctgatgaacctggaggagaagatggacgagctgaaagaggtgttcggactggacgctgaggatctgaacatcgacctgggccctctgggcagcctgctgggtgccccgggatctggcgcaacaaattttagtcttttaaagcaggcaggagacgtcgaggaaaaccctggacccgtggccgtggaacacaacatgcagagcagcaccatcgtggacgtgctggaaaagatcctggacaagggcgtcgtgatcgccggggacatcacagtgggaatcgccgacgtggaactgctgaccatcaagatccggctgatcgtggccagcgtggacaaggccaaagaaatcggcatggattggtgggagaacgacccctacctgagcagcaagggcgccaacaacaaggctctggaagaggaaaacaagatgctgcacgagcggctgaaaacactggaagagaagatcgagacaaagcgcggggcacctggctcgggagcgaccaacttctcattactcaaacaagccggagacgttgaggagaatccaggccctgtgagcaccggccccagcttcagcaccaaggacaacaccctggaatacttcgtgaaggccagcaacaagcacggctttagcctcgacatcagcctgaacgtgaatggggccgtgattagcggcaccatgatcagcgccaaagagtacttcgactacctgagcgagacattcgaagagggcagcgaagtggcccaggccctgtctgagcagtttagcctggctagcgaggcctccgagtctaatggcgaagccgaggcccacttcatccacctgaagaacaccaagatctactgcggcgacagcaagagcacccccagcaagggcaagatcttctggcgcggcaagatcgccgaggtggacggattcttcctgggaaaaatcagcgacgccaagtccaccagcaagaagtccagcggcgctcccgggtcaggtgccacgaatttttcgttgttgaagcaagctggggatgttgaagagaacccagggcctgtggtgtccaagggcgaggaactgttcaccggcgtggtgcccatcctggtggaactggatggcgacgtgaacggccacaagttcagcgtgtccggcgagggcgaaggcgacgccacatacggaaagctgaccctgaagttcatctgcaccaccggcaagctgcccgtgccttggcctaccctcgtgaccacactgacctacggcgtgcagtgcttcgccagataccccgaccacatgaagcagcacgatttcttcaagagcgccatgcccgagggctacgtgcaggaacggaccatcttcttcaaggacgacggcaactacaagacaagagccgaagtgaagttcgagggcgacaccctcgtgaaccggatcgagctgaagggcatcgacttcaaagaggatggcaacatcctgggccacaagctggagtacaactacaacagccacaaggtgtacatcaccgccgacaagcagaaaaacggcatcaaagtgaacttcaagacccggcacaacatcgaggacggcagcgtgcagctggccgaccactaccagcagaacacccccatcggagatggccccgtgctgctgcccgacaaccactacctgagcacacaaagcgccctgagcaaggaccccaacgagaagcgggaccacatggtgctgctggaatttgtgaccgccgctggcatcaccctgggcatggacgagctgtacaagtgactcgagtctagagggccccgtggctgtaatctagaggatccctcgaggggcccaagcttacgcgtgcatgcgacgtcatagctctctccctatagtgagtcgtattataagctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 455 - DNA, 4894 bp, synthetic construct
  misc_feature 1..4894: note = synthetic polynucleotide
  source 1..4894: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctaggcttttgcaaaaagctatttaggtgacactatagaaggtacgcctgcaggtaccgagctcggatccagtacccttcaccatgagcatccagaagtccaccaacagcagcagcctggccgaagtgatcgaccggatcctggacaagggcatcgtgatcgacgccttcgccagagtgtccgtcgtgggcatcgagatcctgaccatcgaggccagagtcgtgatcgccagcgtggacacctggctgagatatgccgaagccgtgggcctgctgcgggacgacgtggaagaaaatggcctgcccgagcggagcaacagctctgagggacagccccggttcagcatctgaactaaatcgcactgtcggcgtccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccgtggccgtggaacacaacatgcagagcagcaccatcgtggacgtgctggaaaagatcctggacaagggcgtcgtgatcgccggggacatcacagtgggaatcgccgacgtggaactgctgaccatcaagatccggctgatcgtggccagcgtggacaaggccaaagaaatcggcatggattggtgggagaacgacccctacctgagcagcaagggcgccaacaacaaggccctggaagaggaaaacaagatgctgcacgagcggctgaaaacactggaagagaagatcgagacaaagcgcggtgccccgggatctggcgcaacaaattttagtcttttaaagcaggcaggagacgtcgaggaaaaccctggacccgtgagcgagacaaacgagacaggcatctacatcttcagcgccatccagacagacaaggatgaggaattcggcgccgtggaagtggaagggaccaaggctgagacattcctgatccggtataaggacgccgccatggtggccgccgaagtgcccatgaagatctaccaccccaaccggcagaacctgctgatgcaccagaatgccgtggccgccatcatggacaagaacgacaccgtgatccccatcagcttcggcaacgtgttcaagagcaaagaggacgtgaaggtgctcctggaaaacctgtacccccagttcgagaagctgttccccgccatcaagggaaagatcgaagtgggcctgaaggtgatcggcaagaaagagtggctcgaaaagaaagtgaacgagaaccccgagctggaaaaagtgtccgccagcgtgaagggcaagagcgaggccgctggctactacgagagaatccagctgggcggcatggcccagaagatgttcacaagcctgcagaaagaagtgaaaaccgacgtgttcagccccctggaagaagccgccgaggccgccaaagccaatgagcctacaggcgaaacaatgctgctgaacgccagcttcctgatcaacagagaggatgaggccaagttcgacgagaaagtcaatgaggcccacgagaactggaaggataaggccgacttccactacagcggcccctggcccgcctacaacttcgtgaacatccggctgaaggtggaagagaagggggcacctggctcgggagcgaccaacttctcattactcaaacaagccggagacgttgaggagaatccaggccctgtgctgcacaagctcgtgaccgcccccatcaacctggtcgtgaagatcggcgagaaggtgcaggaagaggccgacaagcagctgtacgacctgcccaccatccagcagaagctgatccagctgcagatgatgttcgagctgggcgagatccccgaggaagccttccaggaaaaagaggacgaactgctgatgagatacgagatcgccaagcggcgcgagattgagcagtgggaagaactgacccagaagcggaatgaggaaagcggtgccccgggatctggcgcaacaaattttagtcttttaaagcaggcaggagacgtcgaggaaaaccctggacccgtgggcgagctgctgtacctctacggcctgatccccaccaaagaggccgctgctatcgagcccttcccattctacaagggcttcgacggcgagcacagcctgtaccctatcgccttcgaccaagtgaccgccgtggtgttcaagctggacgccgacacctacagcgagaaagtgatccaggaaaagatggaacaggacatgagctggctgcaggaaaaggccttccaccaccacgagacagtggccgccctgtatgaggaattcaccatcatccccctgaagttctgcaccatctataagggagaggaatccctgcaggccgccatcgagatcaacaaagagaagatcgaaaactccctgaccctgctgcagggcaacgaggaatggaacgtgaagatctactgcgacgacaccgagctgaagaagggcatcagcgagacaaacgagagcgtgaaggccaagaagcaggaaatcagccacctgagccccggcagacagttcttcgagaagaagaagattgaccagctcatcgagaaagagctggaactgcacaagaacaaagtgtgcgaggaaatccacgacaagctgattgagctgagcctctacgactccgtgaagaagaactggtccaaggacgtgacaggcgctgccgaacagatggcctggaacagcgtgttcctgctgcccagcctgcagatcaccaagttcgtgaacgagatcgaggaactccagcagcggctggagaacaagggatggaagttcgaagtgaccggcccctggcctccctaccacttcagcagctttgccggggcacctggctcgggagcgaccaacttctcattactcaaacaagccggagacgttgaggagaatccaggccctgtgcagcccgtgtcccaggccaacggcagaatccacctggatcccgatcaggccgaacagggactggcccagctcgtgatgaccgtgatcgagctgctgcggcagatcgtggaacggcacgccatgagaagagtggaaggcggcaccctgaccgacgagcagatcgagaatctgggaatcgccctgatgaacctggaagagaagatggacgagctgaaagaggtgttcggactggacgccgaggacctgaacatcgacctgggccctctgggcagcctgctgtgatcgagtctagagggccccgtggctgtaatctagaggatccctcgaggggcccaagcttacgcgtgcatgcgacgtcatagctctctccctatagtgagtcgtattataagctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 456 - protein, 127 aa, synthetic construct
  REGION 1..127: note = synthetic polypeptide
MGSLTKLLLLPIMGPLNGVVWIAEQIQERTNTEFDAQENLHKQLLSLQLSFDIGEIGEEEFEIQEEEILLKIQALEEEARLELEAEQEEARLELEAEQEDFEYHLNSQQKLIKINISSCYLSIDGRK

SEQ ID NO: 457 - protein, 60 aa, synthetic construct
  REGION 1..60: note = synthetic polypeptide
MPVNKQYQDEQQQVSLCEALDRVLNKGVVIVADITISVANIDLIYLSLQALVSSVEAKNR

SEQ ID NO: 458 - protein, 61 aa, synthetic construct
  REGION 1..61: note = synthetic polypeptide
MSGNKKLTHSTDSTTVADLLERLLDKGVVISGDIRIRLVEVELLTLEIRLLICSVDKAVEM

SEQ ID NO: 459 - protein, 72 aa, synthetic construct
  REGION 1..72: note = synthetic polypeptide
MTVVPAQQTGGGGSSGLYDVLELVLDRGLVIDAFVRVSLVGIEILKIDVRVVVASVDTYLRFAEACNRLDLE

SEQ ID NO: 460 - protein, 72 aa, synthetic construct
  REGION 1..72: note = synthetic polypeptide
MITYDDEVVCAPRAGTLYDVLELILDRGMVIDVFVRVSLVGIEILKVDARIVVASVDTYLRFAEACNRLDLE

SEQ ID NO: 461 - protein, 72 aa, synthetic construct
  REGION 1..72: note = synthetic polypeptide
MTVSSQSMNRAPKPSSLADVLDVVLDRGIVIDAYARVALVGIEVLTADARVVIATVDTYLRFAEAVNRLDLA

SEQ ID NO: 462 - protein, 61 aa, synthetic construct
  REGION 1..61: note = synthetic polypeptide
MTINKSNDCSSLAEVVDRILDKGVVIDVFARISVIGIELITIEARIVIASVDTWLRYAEAV

SEQ ID NO: 463 - protein, 60 aa, synthetic construct
  REGION 1..60: note = synthetic polypeptide
MVSQSPDSSSLAEVLDRILDKGIVVDTWARVSLVGIEILAIEARVVVASVDTFLHYAEEI

SEQ ID NO: 464 - protein, 58 aa, synthetic construct
  REGION 1..58: note = synthetic polypeptide
MAQPDSSSLAEVLDRVLDKGVVVDVYARLSLVGIEILTVEARVVAASVDTFLHYAEEI

SEQ ID NO: 465 - protein, 58 aa, synthetic construct
  REGION 1..58: note = synthetic polypeptide
MVQPDSSSLAEVLDRVLDKGVVVDVWARISLVGIEILTVEARVVAASVDTFLHYAEEI

SEQ ID NO: 466 - protein, 58 aa, synthetic construct
  REGION 1..58: note = synthetic polypeptide
MAQPDSSGLAEVLDRVLDKGVVVDVWARVSLVGIEILTVEARVVAASVDTFLHYAEEI

SEQ ID NO: 467 - protein, 58 aa, synthetic construct
  REGION 1..58: note = synthetic polypeptide
MAQPDSSSLAEVLDRVLDKGVVVDVWARISLVGIEILTVEARVVAASVDTFLHYAEEI

SEQ ID NO: 468 - protein, 61 aa, synthetic construct
  REGION 1..61: note = synthetic polypeptide
MSIQKSTDSSSLAEVIDRILDKGIVIDAFARVSLVGIEILTIEARVVIASVDTWLRYAEAV

SEQ ID NO: 469 - protein, 61 aa, synthetic construct
  REGION 1..61: note = synthetic polypeptide
MSIQKSTNSSSLAEVIDRILDKGIVIDAFARVSVVGIEILTIEARVVIASVDTWLRYAEAV

SEQ ID NO: 470 - protein, 61 aa, synthetic construct
  REGION 1..61: note = synthetic polypeptide
MAVEKTNSSSSLAEVIDRILDKGIVIDAWVRVSLVGIELLAIEARIVIASVETYLKYAEAV

SEQ ID NO: 471 - protein, 61 aa, synthetic construct
  REGION 1..61: note = synthetic polypeptide
MAVEKTNSSSSLAEVIDRILDKGIVIDAWARVSLVGIELLAIEARVVIASVETYLKYAEAV

SEQ ID NO: 472 - protein, 62 aa, synthetic construct
  REGION 1..62: note = synthetic polypeptide
MAKVQKSTDSSSLAEVVDRILDKGIVIDAWVKVSLVGIELLSIEARVVIASVETYLKYAEAI

SEQ ID NO: 473 - protein, 112 aa, Arthrospira platensis
  source 1..112: note = NIES-39
MRYKYHRQIQPKLSAIPRQKSQANLYRNSYLLAVEKKRLTEELEVLQSRSHIIEQRLALIEDQLGELEKDVTQLSVPPSPKPQNNLPVNNPEPPPQSNPTNSSHINTFMVDY

SEQ ID NO: 474-476 - skipped (3 entries)

SEQ ID NO: 477 - DNA, 12 bp, synthetic construct
  misc_feature 1..12: note = synthetic polynucleotide
  source 1..12: mol_type = other DNA
cccttcaccatg

SEQ ID NO: 478 - skipped

SEQ ID NO: 479 - DNA, 57 bp, synthetic construct
  misc_feature 1..57: note = synthetic polynucleotide
  source 1..57: mol_type = other DNA
gctactaacttcagcctccttaaacaggccggcgacgtggaagagaatcctggctag

SEQ ID NO: 480 - DNA, 18 bp, synthetic construct
  misc_feature 1..18: note = synthetic polynucleotide
  source 1..18: mol_type = other DNA
ggagcgccaggttccggg

SEQ ID NO: 481 - DNA, 24 bp, Synthetic construct
  source 1..24: mol_type = other DNA
aaaatggccgcgcccagagcgtag